(19) United States
(12) Patent Application Publication
(10) Pub. No.: US 2026/0364192 A1
(43) Pub. Date: Nov. 19, 2026

(54) CNS TARGETING COMPLEXES AND USES THEREOF

(71) Applicant: Dyne therapeutics, Inc., Waltham, MA (US)

(72) Inventors: Jonathan McNeill, Waltham, MA (US); Stefano Zanotti, Waltham, MA (US); Kim Tang, Belmont, MA (US); JIn Cui, Waltham, MA (US); Susana Correia, Waltham, MA (US); Oxana Beskrovnaya, Waltham, MA (US); Romesh R. Subramanian, Framingham, MA (US); Mohammed T. Qatanani, Waltham, MA (US); TIMOTHY WEEDEN, WALTHAM, MA (US); CODY A DESJARDINS, WALTHAM, MA (US); JOHN NAJIM, WALTHAM, MA (US); BRENDAN QUINN, WALTHAM, MA (US)

(73) Assignee: Dyne therapeutics, Inc., Waltham, MA (US)

(21) Appl. No.: 18/881,000

(22) PCT Filed: Jul. 6, 2023
(86) PCT No.: PCT/US2023/069671

§ 371 Date: Jan. 3, 2025

Related U.S. Application Data
(60) Provisional application No. 63/367,814, filed on Jul. 6, 2022.
(60) Provisional application No. 63/496,184, filed on Apr. 14, 2023.

(51) Int. Cl. A61K 47/68; C07K 16/28; C12N 15/113

(52) U.S. Cl. CPC A61K 47/6807; A61K 47/6849; C07K 16/2881; C12N 15/1138; C07K 2317/55; C07K 2317/565; C12N 2310/11; C12N 2310/315; C12N 2310/322; C12N 2310/3231; C12N 2310/3341; C12N 2310/341; C12N 2310/346; C12N 2310/3513; C12N 2310/3525

(57) ABSTRACT

The present application relates to complexes comprising a central nervous system (CNS)-targeting agent covalently linked to a molecular payload (e.g., an oligonucleotide, polypeptide, small molecule, or gene therapy pay load) for delivery to cells (e.g., cells of the CNS) and uses thereof, particularly uses relating to treatment of disease.

### RELATED APPLICATIONS

This application claims the benefit under 35 U.S.C. § 119(e) of U.S. Provisional Application Ser. No. 63/367,814, entitled “BRAIN TARGETING COMPLEXES AND USES THEREOF,” filed on Jul. 6, 2022, and of U.S. Provisional Application Ser. No. 63/496,184, entitled “CNS TARGETING COMPLEXES AND USES THEREOF,” filed on Apr. 14, 2023, the entire contents of each of which are incorporated herein by reference.

### FIELD OF THE INVENTION

The present application relates to targeting complexes for delivering molecular payloads across the blood-brain barrier and/or to cells of the central nervous system (CNS), formulations comprising such complexes, and uses thereof, particularly uses relating to treatment of disease.

### REFERENCE TO AN ELECTRONIC SEQUENCE LISTING

The contents of the electronic sequence listing (D082470081WO00-SEQ-COB.xml; Size: 3,883,598 bytes; and Date of Creation: Jul. 5, 2023) are herein incorporated by reference in their entirety.

### BACKGROUND

Neurological diseases and disorders, affecting the central nervous system (CNS), affect millions of people around the world, and many have few or no treatment options. Therapeutic compounds with potential efficacy in various neurological conditions often fail to achieve their intended effects or are limited in their efficacy because of limitations in their biodistribution, including inability to efficiently cross the blood-brain barrier.

### SUMMARY

According to some aspects, the present disclosure provides complexes comprising central nervous system (CNS)-targeting agent covalently linked to molecular payloads, compositions comprising such complexes, and methods of their use. The CNS-targeting agents of the complexes described herein comprises an anti-transferrin receptor 1 (TfR1) antibody that is demonstrated to be able to transport the molecular payloads across the blood-brain barrier (e.g., via receptor mediated transcytosis), resulting in delivery of the molecular payloads to cells of the CNS. In some embodiments, the molecular payloads of the complexes described herein modulate the expression or activity of genes associated with disease or disorder of the central nervous system (CNS) and/or have therapeutic effect to a disease or disorder of the CNS.

According to some aspects, complexes are provided herein, wherein a complex comprises an anti-TfR1 antibody covalently linked to a molecular payload for treating a central nervous system (CNS) disease or disorder, wherein the anti-TfR1 antibody comprises:
(i) a heavy chain complementarity determining region 1 (CDR-H1) of SEQ ID NO: 1, a heavy chain complementarity determining region 2 (CDR-H2) of SEQ ID NO: 2, a heavy chain complementarity determining region 3 (CDR-H3) of SEQ ID NO: 3, a light chain complementarity determining region 1 (CDR-L1) of SEQ ID NO: 4, a light chain complementarity determining region 2 (CDR-L2) of SEQ ID NO: 5, and a light chain complementarity determining region 3 (CDR-L3) of SEQ ID NO: 6;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6; or
(iii) a CDR-H1 of SEQ ID NO: 12, a CDR-H2 of SEQ ID NO: 13, a CDR-H3 of SEQ ID NO: 14, a CDR-L1 of SEQ ID NO: 15, a CDR-L2 of SEQ ID NO: 5, and a CDR-L3 of SEQ ID NO: 16;
wherein the complex delivers the molecular payload to a cell of the CNS.

In some embodiments, the anti-TfR1 antibody comprises a heavy chain variable region (VH) comprising an amino acid sequence of SEQ ID NO: 17 and a light chain variable region (VL) comprising an amino acid sequence of SEQ ID NO: 18.

In some embodiments, the anti-TfR1 antibody comprises a heavy chain comprising an amino acid sequence of SEQ ID NO: 19 and a light chain comprising an amino acid sequence of SEQ ID NO: 20.

In some embodiments, the anti-TfR1 antibody is a Fab.

In some embodiments, the molecular payload is configured to modulate expression of a gene associated with the CNS disease or disorder.

In some embodiments, the molecular payload comprises an oligonucleotide, a polypeptide, a small molecule, or a gene therapy payload. In some embodiments, the gene therapy payload comprises a messenger RNA (mRNA) molecule.

In some embodiments, the anti-TfR1 antibody is covalently linked to the molecular payload via a linker comprising a structure of formula (I):

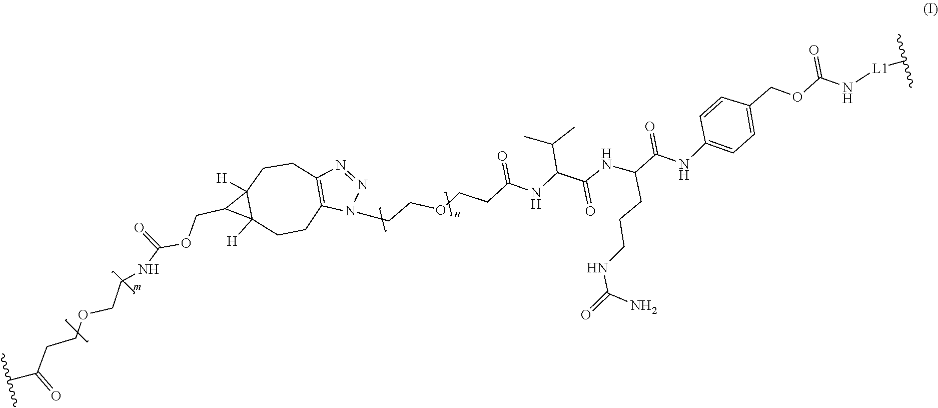

wherein n is any number from 0-10, and wherein m is any number from 0-10;
and wherein L1 is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof, wherein each R^{A} is independently hydrogen or substituted or unsubstituted alkyl. In some embodiments, n is 3 and/or m is 4.

In some embodiments, the complex comprises a structure of formula (J):

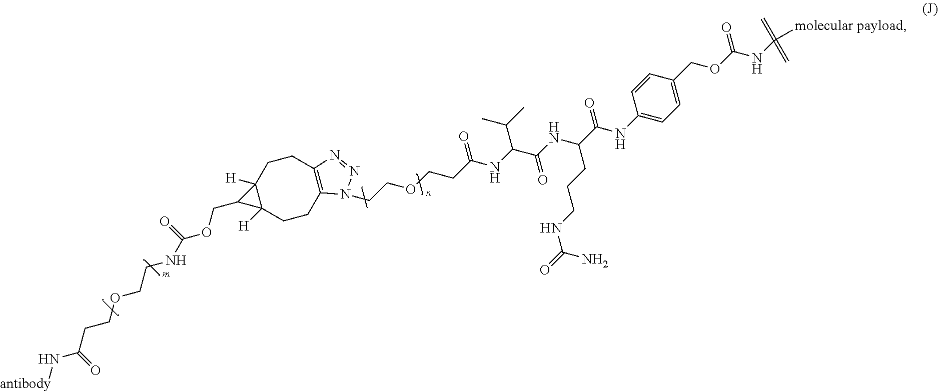

wherein n is any number from 0-10, an wherein m is any number from 0-10. In some embodiments, n is 3 and/or m is 4.

In some embodiments, the complex delivers the molecular payload to the cell of the CNS across the blood-brain barrier. In some embodiments, the complex delivers the molecular payload to the cell of the CNS across the choroid plexus.

In some embodiments, the gene associated with a CNS disease or disorder is DMPK, DMD, SMN, or FXN.

In some embodiments, the gene associated with a CNS disease or disorder is SOD1, C9orf72, ATXN2, or FUS.

In some embodiments, the gene associated with a CNS disease or disorder is LRRK2 or SNCA.

In some embodiments, the gene associated with a CNS disease or disorder is HTT or MSH3.

In some embodiments, the gene associated with a CNS disease or disorder is TREM2, APOE, MAPT, or APP.

In some embodiments, the gene associated with a CNS disease or disorder is GYS1, PrP, VLA-4, GFAP, UBE3A, LSD, or SCN9A.

In some embodiments, the gene associated with a CNS disease or disorder is SCN1A, SCN2A, SCN8A, SCN9A, CLN3, GRIA1, or PCDH19.

In some embodiments, the gene associated with a CNS disease or disorder is: TOR1A, THAP1, ANO3, GNAL, KMT2B, GCH1, TH, SPR, TAF1, PRKRA, ATP1A3, SGCE, PNKD, PRRT2, SLC2A1, or ECHS1.

In some embodiments, the gene associated with a CNS disease or disorder is PIKFYVE, SYF2, or UNC13A.

In some embodiments, the gene associated with a CNS disease or disorder is GRIN2A.

In some embodiments, the gene associated with a CNS disease or disorder is ATXN1, ATXN2, ATXN3, or MSH3.

In some embodiments, the gene associated with a CNS disease or disorder is GRN, C9orf72, MAPT, PIKFYVE, SYF2, or UNC13A.

In some embodiments, the gene associated with a CNS disease or disorder is TPP1 or CLN3.

In some embodiments, the gene associated with a CNS disease or disorder is APOE, SCN1A, GLB1, ASM, ARSA, GALC, HEXA, HEXB, GBA, or MECP2.

In some embodiments, the molecular payload comprises an oligonucleotide comprising a region of complementarity to a transcript as set forth in any one of SEQ ID NOs: 392-702, or to a target sequence of an oligonucleotide listed in any one of Tables 5-19. In some embodiments, the oligonucleotide comprises an oligonucleotide structure listed in any one of Tables 5-19.

In some embodiments, the molecular payload comprises an oligonucleotide comprising a region of complementarity to a transcript as set forth in any one of SEQ ID NOs: 705-803, or to a target sequence of an oligonucleotide listed in any one of Tables 5-19. In some embodiments, the oligonucleotide comprises an oligonucleotide structure listed in any one of Tables 5-19.

In some embodiments, the molecular payload comprises an oligonucleotide comprising a region of complementarity to a transcript as set forth in any one of SEQ ID NOs: 143-148, 167-169, 810-875, and 1059-1068, or to a target sequence of an oligonucleotide listed in any one of Tables 5-19. In some embodiments, the oligonucleotide comprises an oligonucleotide structure listed in any one of Tables 5-19.

In some embodiments, the CNS disease or disorder is a neuromuscular disease or disorder. In some embodiments, the neuromuscular disease or disorder is: Duchenne muscular dystrophy, myotonic dystrophy, Friedreich's ataxia, or spinal muscular atrophy.

In some embodiments, the CNS disease or disorder is amyotrophic lateral sclerosis.

In some embodiments, the CNS disease or disorder is Parkinson's disease.

In some embodiments, the CNS disease or disorder is essential tremor.

In some embodiments, the CNS disease or disorder is Huntington's disease.

In some embodiments, the CNS disease or disorder is Alzheimer's disease.

In some embodiments, the CNS disease or disorder is hereditary dystonia.

In some embodiments, the CNS disease or disorder is epilepsy.

In some embodiments, the CNS disease or disorder is a pain disorder.

In some embodiments, the CNS disease or disorder is a glycogen synthesis disorder; neurodegeneration; small fiber neuropathy; a nociception-related phenotype; Alexander disease; Angelman Syndrome; an autism-spectrum disorder; retinitis pigmentosa; isolated macular dystrophy; and/or multiple sclerosis.

In some embodiments, the CNS disease or disorder is spinocerebellar ataxia (SCA).

In some embodiments, the CNS disease or disorder is frontotemporal dementia (FTD).

In some embodiments, the CNS disease or disorder is motor neuron disease.

In some embodiments, the CNS disease or disorder is Dravet syndrome.

In some embodiments, the CNS disease or disorder is Batten disease.

In some embodiments, the CNS disease or disorder is GM1 gangliosidosis.

In some embodiments, the CNS disease or disorder is Niemann-Pick Type A.

In some embodiments, the CNS disease or disorder is metachromatic leukodystrophy.

In some embodiments, the CNS disease or disorder is Krabbe disease.

In some embodiments, the CNS disease or disorder is Tay-Sachs.

In some embodiments, the CNS disease or disorder is Sandhoff disease.

In some embodiments, the CNS disease or disorder is Gaucher disease, type II or III.

In some embodiments, the CNS disease or disorder is Rett syndrome.

In some embodiments, the CNS disease or disorder is the molecular payload is a molecular payload disclosed in any one of paragraphs [0216]-[1208]. In some embodiments, the molecular payload is a molecular payload disclosed in any one of paragraphs [0296]-[0299], [0404]-[0406], [0468]-[0470], [0500]-[0502], [0535]-[0539], [0601]-[0604], [0666]-[0668], [0757]-[0759], [0779]-[0781], [0896]-[0901], [0916]-[0918], [0946]-[0948], [1049]-[1056], [1070]-[1078], [1092]-[1102], [1116]-[1124], [1138]-[1141], [1155]-[1158], [1172]-[1177], and [1191]-[1193].

According to some aspects, methods of treating a CNS disease or disorder are provided herein, wherein the method comprises administering to a subject in need thereof a complex disclosed herein.

According to some aspects, methods of delivering a molecular payload to the CNS of a subject are provided herein, wherein the method comprises administering to the subject a complex disclosed herein.

In some embodiments, the complex is administered to the subject intravenously.

In some embodiments, the complex is detectable in the cortex of the subject following the administration.

In some embodiments, the complex is detectable in the cerebellum of the subject following the administration.

In some embodiments, the complex is detectable in deep brain tissue of the subject following the administration. In some embodiments, the deep brain tissue is of the thalamus, caudate nucleus and/or putamen of the subject.

In some embodiments, the complex is detectable in cortical neurons, motor neurons, cells of the cerebellum, and/or choroid plexus cells of the subject following the administration.

In some embodiments, the molecular payload comprises a protein. In some embodiments, the protein is an enzyme.

In some embodiments, the subject has been diagnosed with or is suspected of having Batten disease, GM1 gangliosidosis, Niemann-Pick Type A, metachromatic leukodystrophy, Krabbe disease, Tay-Sachs, Sandhoff disease, or Gaucher disease.

In some embodiments, the payload comprises an oligonucleotide.

In some embodiments, the subject has been diagnosed with or is suspected of having ALS, Angelman syndrome, Rett syndrome, Parkinson, lewy body dementia, Alzheimer's disease (which may or may not be associated with cerebral amyloid angiopathy (CAA) or Frontotemporal dementia), epilepsy, Alexander disease, spinal muscular atrophy, Batten disease, Huntington's disease, spinocerebellar ataxia, motor neuron disease, or Dravet syndrome.

### BRIEF DESCRIPTION OF THE DRAWINGS

FIG. 1 shows antisense oligonucleotide (ASO) concentration (ng ASO/g tissue) within brain tissue of mice administered PBS (“Vehicle”), ASO that was not covalently linked to an antibody (“Naked ASO”), complexes comprising a first anti-TfR1 Fab covalently linked to the ASO (“Anti-TfR1 Fab1-ASO Complex”), or complexes comprising a second anti-TfR1 Fab covalently linked to the ASO (“Anti-TfR1 Fab2-ASO Complex”) via intravenous injection. Bars show the average ASO concentration measured +/−standard deviation, and circles represent the values measured in tissues from individual mice (n=4 mice per group).

FIGS. 2A and 2B show antisense oligonucleotide (ASO) concentration (ng ASO/g tissue) within brain tissue of mice administered vehicle control (25 mM Tris, 10% sucrose, pH 7.4; “Vehicle”), ASO that was not covalently linked to an antibody (“Naked ASO”), complexes comprising a control Fab with no specificity for TfR1 covalently linked to the ASO (“Control Fab-ASO Complex”), or complexes comprising an anti-TfR1 Fab covalently linked to the ASO (“Anti-TfR1 Fab1-ASO Complex”) via intravenous injection. FIG. 2A shows ASO concentration measured in the cortex. FIG. 2B shows ASO concentration measured in the cerebellum. Bars show the average ASO concentration measured +standard deviation (n=7 mice per group).

FIGS. 3A and 3B show human mutant DMPK expression measured within brain tissue of mice administered vehicle control (25 mM Tris, 10% sucrose, pH 7.4; “Vehicle”), ASO that was not covalently linked to an antibody (“Naked ASO”), complexes comprising a control Fab with no specificity for TfR1 covalently linked to the ASO (“Control Fab-ASO Complex”), or complexes comprising an anti-TfR1 Fab covalently linked to the ASO (“Anti-TfR1 Fab1-ASO Complex”) via intravenous injection. Values are shown relative to expression in vehicle-treated mice. FIG. 3A shows human mutant DMPK expression measured in the cortex. FIG. 3B shows human mutant DMPK expression measured in the cerebellum. Bars show the average ASO concentration measured +standard deviation (*, P<0.05; ***, P<0.001, calculated by one-way ANOVA with Dunnet's multiple comparisons test; n=7 mice per group).

FIG. 4 shows quantification of human mutant DMPK foci in nuclei of brain cells of the cerebellum of mice administered vehicle control (25 mM Tris, 10% sucrose, pH 7.4; “Vehicle”) or complexes comprising an anti-TfR1 Fab covalently linked to a DMPK-targeting ASO (“Anti-TfR1 Fab1-ASO Complex”) via intravenous injection, measured by counting of foci per mm² of nucleus area over hundreds of cells imaged. Data are shown as mean+standard deviation (*, P<0.05, calculated by Welch's t-test; n=6-7 mice per group).

FIG. 5 shows quantification of human mutant DMPK foci in nuclei of brain cells of the cortex of mice administered vehicle control or complexes comprising an anti-TfR1 Fab covalently linked to a DMPK-targeting ASO (“Anti-TfR1 Fab1-ASO Complex”) via intravenous injection, measured by counting of foci per mm² of nucleus area over hundreds of cells imaged. Data are shown as mean+standard deviation (**, P<0.01; n=4-5 mice per group).

FIG. 6 shows human mutant DMPK expression measured within brain tissue of mice administered vehicle or complexes comprising anti-TfR1 Fab1 covalently linked to DMPK-targeting ASOs. DMPK expression was measured 8 weeks following a single intravenous administration of vehicle or the complexes. Bars show the average DMPK expression normalized to mice administered the vehicle control, +standard deviation (n=3-5 mice per group).

FIG. 7 shows human mutant DMPK expression measured within brain tissue of mice administered vehicle or complexes comprising anti-TfR1 Fab1 covalently linked to DMPK-targeting ASOs. DMPK expression was measured on day 56, following two intravenous administrations (on days 0 and 28, respectively) of vehicle or the complexes. Bars show the average DMPK expression normalized to mice administered the vehicle control, +standard deviation (n=3-5 mice per group; *, P≤0.05).

FIGS. 8A-8C show quantification of ASO (in nM) within brain tissue of cynomolgus monkeys following administration of ASO not covalently linked to an antibody (“Naked ASO”; downward facing arrows) or an ASO-equivalent dose of anti-TfR1 Fab1-ASO complexes (upward facing arrows) via intravenous (IV) injection. ASO content over time was measured by hybridization-based ELISA in the cortex (FIG. 8A), deep brain (FIG. 8B) and cerebellum (FIG. 8C). Values are shown as mean+/−standard error (N=2 monkeys per group).

FIGS. 9A-9C show ASO distribution in brain tissue of cynomolgus monkeys following administration of ASO not covalently linked to an antibody (“Naked ASO”) or an ASO-equivalent dose of anti-TfR1 Fab1-ASO complexes via intravenous (IV) or intrathecal (IT) administration. ASO distribution was measured by in situ hybridization, and is shown in the cortex and deep brain areas (FIG. 9A) and cerebellum (FIG. 9B) in monkeys intravenously (IV) administered either the naked ASO or the complexes, and in the cortex and deep brain of monkeys intrathecally (IT) administered naked ASO or IV administered the complexes (FIG. 9C). The deep brain area shown includes the caudate nucleus and putamen.

FIGS. 10A-10D show ASO concentration (ng ASO/g tissue) within CNS tissue of mice administered vehicle control (25 mM Tris, 10% sucrose, pH 7.4; “Vehicle”) or complexes comprising an anti-TfR1 Fab covalently linked to a DMPK-targeting ASO (“Anti-TfR1 Fab1-ASO Complexes”) at ASO-equivalent doses of 5 mg/kg or 10 mg/kg via intravenous injection at 0 and 28 days. ASO concentration was measured in the cortex (FIG. 10A), cerebellum (FIG. 10B), deep brain regions (FIG. 10C), and spinal cord (FIG. 10D) of treated mice. Bars show the average ASO concentration measured +standard deviation (n=6-7 mice per group).

FIGS. 11A-11E show human mutant DMPK expression measured within brain tissue of mice administered vehicle control (25 mM Tris, 10% sucrose, pH 7.4; “Vehicle”) or complexes comprising an anti-TfR1 Fab covalently linked to the ASO (“Anti-TfR1 Fab1-ASO Complexes”) at ASO-equivalent doses of 5 mg/kg or 10 mg/kg via intravenous injection at 0 and 28 days. Values are shown relative to expression in vehicle-treated mice. Human mutant DMPK expression was measured in the cortex (FIG. 11A), cerebellum (FIG. 11B), deep brain regions (FIG. 11C), brain stem (FIG. 11D) and spinal cord (FIG. 11E) of treated mice. Bars show the average ASO concentration measured +standard deviation (*, P<0.05; **, P<0.01; ***, P<0.005, calculated by one-way ANOVA with Dunnet's multiple comparisons test; n=6-7 mice per group, or 2-3 mice per group for spinal cord analysis).

FIGS. 12A-12C shows quantification of human mutant DMPK foci in nuclei of cortical neurons (FIG. 12A), cells of the cerebellum (FIG. 12B), and choroid plexus cells (FIG. 12C), of mice administered vehicle control (25 mM Tris, 10% sucrose, pH 7.4; “Vehicle”) or complexes comprising an anti-TfR1 Fab covalently linked to a DMPK-targeting ASO (“Anti-TfR1 Fab1-ASO Complexes”) at ASO-equivalent doses of 10 mg/kg via intravenous injection. Bars represent foci area per mm² of nuclear area in regions of interest imaged with an average total tissue area of 2 mm². Data are shown as mean+standard deviation of μm² per mm² (*, P<0.05; **, P<0.01, calculated by Dunnet's multiple comparisons test; n=6-7 mice per group).

### DETAILED DESCRIPTION OF CERTAIN EMBODIMENTS

Aspects of the disclosure relate to a recognition that while certain molecular payloads (e.g., oligonucleotides, peptides, small molecules, gene therapies) can have beneficial effects in cells of the central nervous system (CNS), it has proven challenging for such molecular payloads to achieve their intended effects in the CNS because of limitations in their biodistribution, including inability to efficiently cross the blood-brain barrier. Accordingly, the present disclosure, in some aspects, provides complexes comprising CNS-targeting agents covalently linked to molecular payloads in order to overcome such challenges. The CNS-target agent of the present disclosure comprises an anti-transferrin receptor 1 (TfR1) antibody that is demonstrated to be able to transport molecular payloads to cells of the CNS. In some embodiments, such delivery is across the blood-brain barrier (e.g., via receptor mediated transcytosis), resulting in delivery of the molecular payloads to cells of the CNS. In some embodiments, such delivery is across the choroid plexus, resulting in delivery of the molecular payloads to cells of the CNS. In some embodiments, an anti-TfR1 antibody of the complexes described herein exhibits pH-dependent binding affinity to TfR1 (e.g., having different binding affinity under different pH conditions). In some embodiments, an anti-TfR1 antibody of the complexes described herein exhibits pH-independent binding affinity to TfR1 (e.g., having comparable binding affinity under different pH conditions).

In some embodiments, complexes provided herein may comprise molecular payloads that modulate (e.g., increase or reduce) expression and/or activity of genes associated with CNS diseases and disorders, such as by modulating transcription, translation, post-transcriptional modification (e.g., splicing), mRNA stability, and/or protein stability. In some embodiments, complexes provided herein may comprise molecular payloads that are synthetic nucleic acids (e.g., DNA or RNA) that may be used to express one or more proteins that modulate expression and activity of genes associated with CNS diseases and disorders. In some embodiments, complexes provided herein may comprise molecular payloads that have therapeutic effect in a CNS disease or disorder, but may or may not modulate the expression or activity of any genes associated with CNS diseases and disorders.

Neurological diseases and disorders, which affect the CNS, have varying etiologies and potential treatment modalities. Examples of such CNS diseases and disorders include, without limitation, neuromuscular disorders (e.g., myotonic dystrophy, Duchenne muscular dystrophy, Friedreich's ataxia, and spinal muscular atrophy), amyotrophic lateral sclerosis (ALS), frontotemporal dementia (FTD), Parkinson's disease, Huntington's disease, Alzheimer's disease, epilepsy, and pain disorders, among others. Other examples of CNS diseases and disorders include essential tremor and hereditary dystonia. Additional examples of CNS diseases and disorders include spinocerebellar ataxia, motor neuron disease, Dravet syndrome, Batten disease, GM1 gangliosidosis, Niemann-Pick Type A, metachromatic leukodystrophy, Krabbe disease, Tay-Sachs, Sandhoff disease, Gaucher disease types II and III, and Rett syndrome. Various genes are implicated in CNS diseases and disorders, including, without limitation, DMPK, DMD, SMN, FXN, SOD1, C9orf72, ATXN2, FUS, LRRK2, SNCA, HTT, MSH3, TREM2, APOE, MAPT, APP, GYS1, PrP, VLA-4, UBE3A, GFAP, LSD, SCN9A, SCN1A, SCN2A, SCN8A, CLN3, GRIA1, and PCDH19, among others. Other genes implicated in CNS diseases and disorders include, without limitation, TOR1A, THAP1, ANO3, GNAL, KMT2B, GCH1, TH, SPR, TAF1, PRKRA, ATP1A3, SGCE, PNKD, PRRT2, SLC2A1, and ECHS1. Additional genes implicated in CNS diseases and disorders include, without limitation, PIKFYVE, SYF2, UNC13A, ATXN1, ATXN3, GRN, GRIN2A, TPP1, GLB1, ASM, ARSA, GALC, HEXA, HEXB, GBA, and MECP2. Molecular payloads may be effective at treating CNS diseases and disorders upon their delivery to cells of the CNS, e.g., by crossing the blood-brain barrier, and/or by crossing the choroid plexus. Certain molecular payloads may alleviate signs or symptoms of CNS diseases and disorders, such as, in some embodiments, by modulating expression or activity of genes implicated in CNS diseases and disorders. Molecular payloads that alleviate signs or symptoms of CNS diseases and disorders without modulating expression or activity of any genes implicated in CNS diseases and disorders may also be used in accordance with the present disclosure. Delivery of molecular payloads to the CNS may also be useful for other purposes aside from treatment of CNS diseases and disorders.

Further aspects of the disclosure, including a description of defined terms, are provided below.

### I. Definitions

Administering: As used herein, the terms “administering” or “administration” means to provide a complex to a subject in a manner that is physiologically and/or (e.g., and) pharmacologically useful (e.g., to treat a condition in the subject).

Alzheimer's Disease: As used herein, the term “Alzheimer's disease” refers to a progressive neurological disorder that is characterized by atrophy of brain tissue and loss of neurons, particularly with advanced age. Alzheimer's disease is a frequent cause of dementia, including pre-senile dementia. Symptoms of Alzheimer's disease include memory loss that worsens over time, difficulty with concentration, especially in regard to abstract concepts, difficulty with multitasking, impaired decision-making, and changes in personality or behavior. Alzheimer's disease is also associated with the formation of beta-amyloid protein plaques and tau protein tangles (also known as neurofibrillary tangles) in brain tissue, which are cytotoxic, disrupt communication between cells, and contribute to neuronal death. The cause of Alzheimer's disease is incompletely understood, however, development of Alzheimer's disease can be influenced by inheritance of certain genetic risk factors. For example, genes involved in the pathophysiology of Alzheimer's disease include, but are not limited to, TREM2, APOE, MAPT, and APP (see, e.g., Neuner S M, et al. “Genetic architecture of Alzheimer's disease.” Neurobiol Dis. 2020; 143:104976; and Ibanez L, et al. “Advances in Genetic and Molecular Understanding of Alzheimer's Disease.” Genes (Basel). 2021; 12(8):1247). Alzheimer's disease may or may not be associated with cerebral amyloid angiopathy (CAA) or Frontotemporal dementia.

Amyotrophic lateral sclerosis (ALS): Amyotrophic lateral sclerosis (ALS) is a progressive neurodegenerative disease that affects nerve cells of the central nervous system. ALS results in degeneration of motor neurons which control muscle movement, ultimately resulting in loss of control of the muscles needed to move, speak, eat, and breathe. Approximately 90% of ALS cases are considered sporadic, occurring in patients without a known family history of the disease, and 5-10% of all cases are familial (i.e., inherited). Genes associated with the development of ALS include, for example, SOD1 (associated with about 12-20% of familial ALS cases), C9orf72 (associated with about 25-40% of familial ALS cases), ATXN2, and FUS. In some embodiments, accumulation of TDP-43 aggregates is associated with ALS. PIKFYVE, SYF2, and UNC13A have also been implicated in the pathophysiology of ALS. In some embodiments, single nucleotide polymorphism(s) and/or other alterations in PIKFYVE, SYF2, and UNC13A are associated with ALS.

ANO3: As used herein, ANO3 refers to the gene encoding Anoctamin 3 (also referred to as DYT23; DYT24; TMEM16C; C11orf25; or GENX-3947), a protein that belongs to the TMEM16 family of predicted membrane proteins. In some embodiments, ANO3 may be a human (Gene ID: 63982), non-human primate (e.g., Gene ID: 101865236), or rodent gene (e.g., Gene ID: 228432, Gene ID: 311287). In humans, mutations in a gene encoding ANO3 are associated with the development of hereditary dystonia. In addition, multiple human transcript variants (e.g., as annotated under GenBank RefSeq Accession Numbers: NM_001313726.2; NM_031418.4; XM_047427399.1; XM_017018118.3; NM_001313727.2; XM_017018119.3; and XM_011520282.4) have been characterized that encode different protein isoforms.

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

APP: As used herein, the term “APP” refers to the gene encoding amyloid beta precursor protein (also referred to as AAA, ABETA, ABPP, AD1, APPI, CTFgamma, CVAP, PN-II, PN2, alpha-sAPP, and pre-A4), a protein involved in synapse formation and neural plasticity. In some embodiments, APP may be a human (Gene ID: 351), non-human primate (e.g., Gene ID: 100427716), or rodent gene (e.g., Gene ID: 11820, Gene ID: 54226). In humans, mutations in an APP gene are associated with the development of Alzheimer's disease. In addition, multiple human transcript variants (e.g., as annotated under GenBank RefSeq Accession Numbers: NM_000484.4 and NM_201413.3) have been characterized that encode different protein isoforms.

APOE: As used herein, APOE refers to the gene encoding apolipoprotein E (also referred to as AD2, ApoE4, LDLCQ5, and LPG), a protein involved in the formation of lipoprotein particles and the transport of lipids through the circulatory system. In some embodiments, APOE may be a human (Gene ID: 348), non-human primate (e.g., Gene ID: 714623), or rodent gene (e.g., Gene ID: 11816, Gene ID: 25728). In humans, mutations in a gene encoding APOE are associated with the development of Alzheimer's disease. In some embodiments, APOE4 allele is associated with the development of Alzheimer's disease. In some embodiments, APOE4 allele is associated with the development of motor neuron disease. Accordingly, in some embodiments, allele-specific modulation (e.g., suppression) of APOE (e.g., APOE4) is useful in the treatment of CNS disease or disorders such as Alzheimer's disease or motor neuron disease. In addition, multiple human transcript variants (e.g., as annotated under GenBank RefSeq Accession Numbers: NM_000041.4 and NM_001302688.2) have been characterized that encode different protein isoforms.

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

ARSA: As used herein, ARSA refers to a gene encoding arylsulfatase A (also referred to as, Cerebroside-Sulfatase, Epididymis Secretory Sperm Binding Protein, MLD, ASA, or sulfatidase) an enzyme that breaks down sulfatides. In some embodiments ARSA may be a human (e.g., Gene ID: 410), non-human primate (e.g., Gene ID: 458946, Gene ID: 716500), or rodent (e.g., Gene ID: 11883, Gene ID: 315222) gene. In humans, mutations in a gene encoding ARSA are associated with the development of Metachromatic leukodystrophy. In addition, multiple human transcript variants (e.g., as annotated under GenBank RefSeq Accession Numbers: NM_000487.6; NM_001085425.3; NM_001085426.3; NM_001085427.3; NM_001085428.3; NM_001362782.2; XM_047441363.1; XM_024452241.2; XM_011530691.4) have been characterized that encode different protein isoforms.

ASM: As used herein, the term “ASM” refers to a gene (also known as SCMPD1, ASMASE, and NPD) encoding acid sphingomyelinase. Wildtype acid sphingomyelinase is a lysosomal enzyme involved in the conversion of lipids into ceramide. Mutations in ASM typically result in a shortage or complete loss-of-function of acid sphingomyelinase, leading to accumulation of fat in the cells of various organs and tissues, including the central nervous system. In some embodiments, ASM may be a human gene (Gene ID: 6609), a non-human primate gene (Gene ID: 711248) or a rodent gene (Gene ID: 20597; Gene ID: 308909). In humans, mutations in ASM are associated with the development of Niemann-Pick Type A.

ATP1A3: As used herein, ATP1A3 refers to the gene encoding ATPase Na+/K+ transporting subunit alpha 3 (also referred to as RDP; AHC2; CAPOS; DEE99; DYT12; or ATP1A1), a protein that belongs to the family of P-type cation transport ATPases, and to the family of Na+/K+ ATPases. In some embodiments, ATP1A3 may be a human (Gene ID: 478), non-human primate (e.g., Gene ID: 102122869), or rodent gene (e.g., Gene ID: 232975, Gene ID: 24213). In humans, mutations in a gene encoding ATP1A3 are associated with the development of hereditary dystonia. In addition, multiple human transcript variants (e.g., as annotated under GenBank RefSeq Accession Numbers: NM_152296.5; NM_001256213.2; NM_001256214.2; and XM_047438862.1) have been characterized that encode different protein isoforms.

ATXN1: As used herein, ATXN1 refers to a gene that encodes the protein ataxin-1. Ataxin-1 protein is expressed throughout the body, and is thought to be involved in regulating protein production, including transcription and RNA processing. Ataxin-1 binds RNA and associates with large protein complexes. It is thought to be involved in transcriptional repression and to regulate Notch- and Capicua-controlled developmental processes. Human ATXN1 (Gene ID: 6310) includes a CAG repeat region, which normally includes 6-39 repeats. Longer expansions of the CAG repeat region (typically 40-83 or more) in ATXN1 can result in neurodegenerative disease, including spinocerebellar ataxia 1 (SCA1). The expanded CAG repeat region results in incorrect protein folding and a nonfunctional ataxin-1 protein. The abnormal protein forms aggregates within cell nuclei, resulting in cell damage. Evidence suggests that ataxin-1 aggregates are found only or primarily in cells of CNS, and particularly within Pukinje cells of the cerebellum. Accumulation of protein aggregates results in cell death; the loss of these cells over time results in the cerebellar deficiencies characteristic of SCA1. See Banfi, et al. “Identification and characterization of the gene causing type 1 spinocerebellar ataxia” Nature Genet. 7:513-520 (1994) and Orr, et al. “Expansion of an unstable trinucleotide CAG repeat in spinocerebellar ataxia type 1” Nature Genet. 4: 221-226 (1993). Allele-specific inhibition of ATXN1 may be effective in treatment of SCA1. See Miller, et al. “Allele-specific silencing of dominant disease genes” Proc. Nat. Acad. Sci. 100: 7195-7200 (2003).

ATXN2: As used herein, ATXN2 refers to the gene that encodes the protein ataxin-2. It is ubiquitously expressed in various tissues, and the ataxin-2 protein localizes to the Golgi apparatus and stress granules within normal cells. The ataxin-2 protein is involved in regulating mRNA translation through its interactions with the poly(A)-binding protein, and is also involved in the formation of stress granules and P-bodies, both of which are also involved in RNA regulation. Human ATXN2 includes a CAG repeat region, which normally includes 22 or 23 repeats, but can include up to 31 repeats. Longer expansions of the CAG repeat region in ATXN2 can result in neurodegenerative disease, including spinocerebellar ataxia 2 (SCA2) and amyotrophic lateral sclerosis (ALS). Interactions between ataxin-2 and the protein TDP-43 are thought to be involved in the development of ALS in certain patients. See, e.g., Elden, et al. “Ataxin-2 intermediate-length polyglutamine expansions are associated with increased risk for ALS” Nature 466:1069-1075 (2010). A disease-associated ATXN2 allele often contains 34-52 CAG repeats, but can contain as few as 32 or over 100, and can expand in size when the allele is transmitted to successive generations. In some embodiments, as few as 27 CAG repeats can be associated with ALS, as described in Elden, et al. Allele-specific inhibition of ATXN2 may be effective in treatment of SCA2. See Miller, et al. “Allele-specific silencing of dominant disease genes” Proc. Nat. Acad. Sci. 100: 7195-7200 (2003).

ATXN3: As used herein, ATXN3 refers to a gene (also known as AT3, ATX3, JOS, MJD, MJD1, and SCA3) which encodes ataxin-3 protein. Ataxin-3 protein is expressed throughout the body, and is believed to be involved in the proteasome processing system. Ataxin-3 removes ubiquitin from proteins to be degraded so that the ubiquitin can be recycled. Ataxin-3 may also be involved in regulating the first stage of transcription. Human ATXN2 (Gene ID: 4287) includes a CAG repeat region, which normally includes 13-36 repeats. Longer expansions of the CAG repeat region (typically 50 or more) in ATXN3 can result in neurodegenerative disease, including spinocerebellar ataxia 3 (SCA3). The expanded CAG repeat region results in incorrect protein folding and a nonfunctional ataxin-3 protein. This nonfunctional ataxin-3 protein cannot remove ubiquitin from proteins, resulting in aggregation of such proteins, along with ubiquitin and ataxin-3, within the nucleus of cells. These protein aggregates can result in cell death, including in neurons and other cells of the CNS. Neurons are typically the cell types that are the most affected by mutations in ATXN3. See Kawaguchi, et al. “CAG expansions in a novel gene for Machado-Joseph disease at chromosome 14q32.1” Nature Genet. 8: 221-228 (1994). Allele-specific inhibition of ATXN3 may be effective in treatment of SCA3. See Miller, et al. “Allele-specific silencing of dominant disease genes” Proc. Nat. Acad. Sci. 100: 7195-7200 (2003).

Batten disease: As used herein, the term “Batten disease” refers to a family of lysosomal disorders also known as neuronal ceroid lipofuscinoses (NCLs). Batten diseases are nervous system disorders caused by various mutations to 13 genes, usually inherited in a recessive pattern. In some embodiments, Batten disease results from mutations in CLN1, CLN2, CLN3, CLN4, CLN5, CLN6, CLN7, CLN8, CLN10, CLN11, CLN12, CLN13, or CLN14. In particular, as relevant to the present disclosure, in some embodiments Batten disease results from mutations in CLN2 or CLN3. Symptoms of Batten disease include seizures, visual impairment, cognitive and behavioral decline, motor decline, developmental impairment, and premature death. Batten disease is characterized by lysosomal accumulation of autofluorescent storage material, glial reactivity, and neuronal loss. The genetic causes of Batten disease are well known and are attributed to mutations in one of thirteen different genes encoding lyososomal and extralysosomal proteins. Genes involved in the pathophysiology of Batten disease include PPT1 (CLN1), TPP1 (CLN2), CLN3 (CLN3), DNAJC5 (CLN4), CLN5 (CLN5), CLN6 (CLN6), MFSD8 (CLN7), CLN8 (CLN8), CTSD (CLN10), GRN (CLN11), ATP13A2 (CLN12), CTSF (CLN13), and KCTD7 (CLN14). In some embodiments, a subject in need of treatment for Batten disease presents with seizure activity. In some embodiments, seizure activity comprises myoclonic jerks, grand mal seizures, and tonic-clonic seizures. In some embodiments, a subject in need of treatment for Batten disease exhibits symptoms of visual impairment. In some embodiments, visual impairment comprises optic nerve atrophy, progressive vision loss, pigmentary retinopathy, macular degeneration, visual failure, retinopathy, diminished pupillary light reflex, central vision loss, and blindness. In some embodiments, a subject in need of treatment for Batten disease exhibits symptoms of cognitive and behavioral decline. In some embodiments, cognitive and behavioral decline comprises irritability, hyperexcitability, anxiety, agitation, depression, inappropriate laughter, mood disturbances, intellectual disability, dementia, and personality abnormalities. In some embodiments, a subject in need of treatment for Batten disease exhibits symptoms of motor decline. In some embodiments, motor decline comprises loss of motor coordination, choreoathetosis, stereotypic movements, myoclonus ataxia, motor decline, spasticity, dystonic features, hypotonia, rigidity, impaired balance, myoclonus, ataxia, facial dyskinesia, clumsiness, motor coordination loss, dysarthria, severe respiratory distress, central, axial and/or limb hypotonia, appendicular spasticity, tremor, parkinsonism, hyperreflexia, speech apraxia, echolalia, delayed speech, and dysarthric speech. In some embodiments, a subject in need of treatment for Batten disease exhibits symptoms of developmental impairment. In some embodiments, developmental impairment comprises decelerated head growth, premature death, microcephaly, overriding sutures, halt in developmental milestones, developmental regression, and developmental arrest.

Blood-brain barrier: as used herein, the term “blood-brain barrier” refers to a highly selective semipermeable border of endothelial cells that prevents various molecules in the blood from non-selectively crossing into the extracellular fluid of the CNS. It allows passage of some small molecules by diffusion, and active transport of various nutrients, ions, organic anions, and macromolecules such as glucose and amino acids that are crucial to neural function, but blocks non-specific transport of other molecules.

C9orf72: As used herein, C9orf72 refers to the gene which encodes the chromosome 9 open reading frame 72 protein. The protein is found in many regions of the brain, including within the cytoplasm of neurons and in presynaptic terminals. Disease-causing mutations, particularly hexanucleotide repeat expansions, in the C9orf72 gene are associated with ALS. In some embodiments, mutations in C9orf72 are associated with familial forms of ALS. In some embodiments, mutations in C9orf72 are associated with frontotemporal dementia (e.g., C9FTD).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the International ImMunoGeneTics Information System® imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

CDR-grafted antibody: The term “CDR-grafted antibody” refers to antibodies which comprise heavy and light chain variable region sequences from one species but in which the sequences of one or more of the CDR regions of VH and/or (e.g., and) VL are replaced with CDR sequences of another species, such as antibodies having murine heavy and light chain variable regions in which one or more of the murine CDRs (e.g., CDR3) has been replaced with human CDR sequences.

Central nervous system (CNS): as used herein, the term “central nervous system” (CNS) refers to the brain and spinal cord, and includes neurons and non-nervous supporting cells (e.g., glia) as well as the blood-brain barrier cells. The blood-brain barrier prevents various molecules from non-selectively crossing into the extracellular fluid of the CNS from the circulation. The CNS also includes cells of the blood-cerebrospinal fluid barrier, such as the cells of the choroid plexus.

CNS disease or disorder: as used herein, a “CNS disease or disorder” refers to a disease or disorder which affects the CNS (e.g., CNS structure or function) and/or which has an etiology in the CNS. CNS diseases and disorders are also known as neurological disease or disorders. Examples of CNS diseases or disorders include, but are not limited to, neuromuscular diseases and disorders (e.g., muscular dystrophy, myotonic dystrophy, spinal muscular atrophy, and Friedreich's ataxia), amyotrophic lateral sclerosis (ALS), Parkinson's disease, Huntington's disease, Alzheimer's disease, epilepsy, and pain disorders, amongst others. Other examples of CNS diseases or disorders include essential tremor and hereditary dystonia. Certain lysosomal storage disorders are also examples of CNS diseases or disorders.

CNS-targeting agent: As used herein, the term, “CNS-targeting agent,” refers to a molecule that specifically binds to an antigen expressed on cells of the CNS (e.g., neurons, supporting cells, and/or cells of the blood-brain barrier). The antigen in or on CNS cells may be a membrane protein, for example an integral membrane protein or a peripheral membrane protein. Typically, a CNS-targeting agent specifically binds to an antigen on CNS cells that facilitates transport of the molecular payload across the blood-brain barrier and/or internalization of the CNS-targeting agent (and any associated molecular payload) into the CNS cells. In some embodiments, a CNS-targeting agent specifically binds to an internalizing, cell surface receptor (e.g., transferrin receptor 1) on cells of the CNS and is capable of being internalized into CNS cells through receptor mediated internalization. In some embodiments, a CNS-targeting agent is a small molecule, a protein, a peptide, a nucleic acid (e.g., an aptamer), or an antibody. In some embodiments, a CNS-targeting agent is linked to a molecular payload.

CNS-targeting antibody: As used herein, the term “CNS-targeting antibody” refers to a CNS-targeting agent that is an antibody that specifically binds to an antigen found in or on CNS cells. In some embodiments, a CNS-targeting antibody specifically binds to an antigen on CNS cells (e.g., neurons, supporting cells, and/or cells of the blood-brain barrier) that facilitates transport of the molecular payload across the blood-brain barrier and/or internalization of the CNS-targeting antibody (and any associated molecular payment) into the CNS cells. In some embodiments, the CNS-targeting antibody specifically binds to an internalizing, cell surface receptor present on CNS cells. In some embodiments, the CNS-targeting antibody facilitates transcytosis across cells (e.g., endothelial cells) of the blood-brain barrier. In some embodiments, the CNS-targeting antibody is an antibody that specifically binds to a transferrin receptor (e.g., transferrin receptor 1).

Chimeric antibody: The term “chimeric antibody” refers to antibodies which comprise heavy and light chain variable region sequences from one species and constant region sequences from another species, such as antibodies having murine heavy and light chain variable regions linked to human constant regions.

CLN2: See “TPP1”.

CLN3: As used herein, the term “CLN3” refers to the gene encoding CLN3 lysosomal/endosomal transmembrane protein (also referred to as battenin, BTN1, BTS, and JNCL), a protein involved in lysosomal function. In some embodiments, CLN3 may be a human (Gene ID: 1201), non-human primate (e.g., Gene ID: 705815), or rodent gene (e.g., Gene ID: 12752, Gene ID: 293485). In humans, mutation in a gene encoding CLN3 is associated with epilepsy and seizures, as well as CLN3 Batten disease. In addition, multiple human transcript variants (e.g., as annotated under GenBank RefSeq Accession Numbers: NM_000086.2 and NM_001286104.2) have been characterized that encode different protein isoforms.

Complementary: As used herein, the term “complementary” refers to the capacity for precise pairing between two nucleosides or two sets of nucleosides. In particular, complementary is a term that characterizes an extent of hydrogen bond pairing that brings about binding between two nucleosides or two sets of nucleosides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., a CNS-targeting agent, such as an antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferrin receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

DMD: As used herein, the term “DMD” refers to a gene that encodes dystrophin protein, a key component of the dystrophin-glycoprotein complex, which bridges the inner cytoskeleton and the extracellular matrix in muscle cells, particularly muscle fibers. Deletions, duplications, and point mutations in DMD may cause dystrophinopathies, such as Duchenne muscular dystrophy, Becker muscular dystrophy, or cardiomyopathy. Alternative promoter usage and alternative splicing result in numerous distinct transcript variants and protein isoforms for this gene. In some embodiments, a dystrophin gene may be a human (Gene ID: 1756), non-human primate (e.g., Gene ID: 465559), or rodent gene (e.g., Gene ID: 13405; Gene ID: 24907). In addition, multiple human transcript variants (e.g., as annotated under GenBank RefSeq Accession Numbers: NM_000109.3, NM_004006.2, NM_004009.3, NM_004010.3 and NM_004011.3) have been characterized that encode different protein isoforms.

DMD allele: As used herein, the term “DMD allele” refers to any one of alternative forms (e.g., wild-type or mutant forms) of a DMD gene. In some embodiments, a DMD allele may encode for dystrophin that retains its normal and typical functions. In some embodiments, a DMD allele may comprise one or more mutations that results in muscular dystrophy. Common mutations that lead to Duchenne muscular dystrophy involve frameshift, deletion, substitution, and duplicative mutations of one or more of 79 exons present in a dystrophin allele, e.g., exon 8, exon 23, exon 41, exon 44, exon 45, exon 50, exon 51, exon 52, exon 53, or exon 55. Further examples of DMD mutations are disclosed, for example, in Flanigan K M, et al., Mutational spectrum of DMD mutations in dystrophinopathy patients: application of modern diagnostic techniques to a large cohort. Hum Mutat. 2009 December; 30 (12):1657-66, the contents of which are incorporated herein by reference in its entirety.

DMPK: As used herein, the term “DMPK” refers to a gene that encodes myotonin-protein kinase (also known as myotonic dystrophy protein kinase or dystrophia myotonica protein kinase), a serine/threonine protein kinase. Substrates for this enzyme may include myogenin, the beta-subunit of the L-type calcium channels, and phospholemman. In some embodiments, DMPK may be a human (Gene ID: 1760), non-human primate (e.g., Gene ID: 456139, Gene ID: 715328), or rodent gene (e.g., Gene ID: 13400). In humans, a CTG repeat expansion in the 3′ non-coding, untranslated region of DMPK is associated with myotonic dystrophy type I (DM1). In addition, multiple human transcript variants (e.g., as annotated under GenBank RefSeq Accession Numbers: NM_001081563.2, NM_004409.4, NM_001081560.2, NM_001081562.2, NM_001288764.1, NM_001288765.1, and NM_001288766.1) have been characterized that encode different protein isoforms.

DMPK allele: As used herein, the term “DMPK allele” refers to any one of alternative forms (e.g., wild-type or mutant forms) of a DMPK gene. In some embodiments, a DMPK allele may encode for wild-type myotonin-protein kinase that retains its normal and typical functions. In some embodiments, a DMPK allele may comprise one or more disease-associated-repeat expansions. In some embodiments, normal subjects have two DMPK alleles comprising in the range of 5 to 37 repeat units. In some embodiments, the number of CTG repeat units in subjects having DM1 is in the range of about 50 to about 3,000 or more with higher numbers of repeats leading to an increased severity of disease. In some embodiments, mildly affected DM1 subjects have at least one DMPK allele having in the range of 50 to 150 repeat units. In some embodiments, subjects with classic DM1 have at least one DMPK allele having in the range of 100 to 1,000 or more repeat units. In some embodiments, subjects having DM1 with congenital onset may have at least one DMPK allele comprising more than 2,000 repeat units.

Dravet syndrome: As used herein, the term “Dravet syndrome”, also known as severe myoclonic epilepsy of infancy (SMEI), refers to the most severe disorder in the genetic epilepsy with febrile seizures plus (GEFS+) spectrum. Dravet syndrome is typically caused by de novo mutations, but cases arising from familial mutations also occur. Symptoms of Dravet syndrome include seizures, cognitive decline, developmental regression, intellectual disability, and ataxia. More than 80% of Dravet syndrome cases are attributed to mutations in SCN1A, in which over 900 distinct mutations have been reported. In some embodiments, a subject in need of treatment for Dravet syndrome has symptoms selected from: seizures (e.g., febrile seizures, afebrile seizures, myoclonic seizures and absence seizures), cognitive decline, developmental regression, intellectual disability, and ataxia.

Dystrophinopathy: As used herein, the term “dystrophinopathy” refers to a muscle or neurological disease results from one or more mutated DMD alleles. Dystrophinopathies include a spectrum of conditions (ranging from mild to severe) that includes Duchenne muscular dystrophy, Becker muscular dystrophy, and DMD-associated dilated cardiomyopathy (DCM). In some embodiments, at one end of the spectrum, dystrophinopathy is phenotypically associated with an asymptomatic increase in serum concentration of creatine phosphokinase (CK) and/or (e.g., and) muscle cramps with myoglobinuria. In some embodiments, at the other end of the spectrum, dystrophinopathy is phenotypically associated with progressive muscle diseases that are generally classified as Duchenne or Becker muscular dystrophy when skeletal muscle is primarily affected and as DMD-associated dilated cardiomyopathy (DCM) when the heart is primarily affected. Symptoms of Duchenne muscular dystrophy include muscle loss or degeneration, diminished muscle function, pseudohypertrophy of the tongue and calf muscles, higher risk of neurological abnormalities, and a shortened lifespan. Duchenne muscular dystrophy is associated with Online Mendelian Inheritance in Man (OMIM) Entry #310200. Becker muscular dystrophy is associated with OMIM Entry #300376. Dilated cardiomyopathy is associated with OMIM Entry X #302045.

ECHS1: As used herein, ECHS1 refers to the gene encoding enoyl-CoA hydratase, short chain 1 (also referred to as SCEH; mECH; mECH1; or ECHS1D), a protein that functions in the second step of the mitochondrial fatty acid beta-oxidation pathway. In some embodiments, ECHS1 may be a human (Gene ID: 1892), non-human primate (e.g., Gene ID: 101925228), or rodent gene (e.g., Gene ID: 93747, Gene ID: 140547). In humans, mutations in a gene encoding ECHS1 are associated with the development of hereditary dystonia. In addition, multiple human transcript variants (e.g., as annotated under GenBank RefSeq Accession Number NM_004092.4) have been characterized that encode different protein isoforms.

Epilepsy: As used herein, the term “epilepsy” refers to a neurological disorder that is characterized by abnormal activity in neurons of the brain, which can cause episodic seizures and may be accompanied by loss of consciousness. Symptoms of epileptic seizures may include sudden confusion or anxiety, dizziness, loss of awareness, loss of consciousness, staring spells, muscular stiffness, and involuntary movement of extremities. Epileptic seizure frequently occur in the absence of an external stimulus. Epileptic seizures can occur due to abnormal neurological activity in only brain region (focal seizure), or due to abnormal neurological activity throughout the brain (generalized seizure). Focal seizures may occur with or without loss of consciousness and may cause altered muscle movement and/or sensory perception. Generalized seizures are further characterized as absence seizures (i.e., petit mal seizures, which occur briefly and cause loss of awareness and repetitive body movements), clonic seizures (repetitive involuntary muscle movements), myoclonic seizures (sudden involuntary muscle movements), tonic seizures (muscle stiffness), atonic seizures (loss of muscle control, without stiffness), and tonic-clonic seizures (i.e., grand mal seizures, which cause loss of consciousness, muscle stiffness, and sudden involuntary muscle movements). Epilepsy may cause death due to injuries sustained during a seizure, status epilepticus, or sudden unexpected death in epilepsy (SUDEP). Only approximately half of epilepsy cases have an identifiable cause, such as a developmental disorder, a brain abnormality (e.g., brain cancer, traumatic brain injury, or a vascular disorder, e.g., stroke), an infection affecting the brain, or inheritance of a genetic risk factor. For example, genes involved in the pathophysiology of epilepsy include, but are not limited to, SCN1A, SCN2A, SCN8A, CLN3, GRIA1, and PCDH19 (see, e.g., Wang J, et al. “Epilepsy-associated genes.” Seizure. 2017; 44:11-20; Abdennadher M, et al. “Seizure phenotype in CLN3 disease and its relation to other neurologic outcome measures.” J Inherit Metab Dis. 2021; 44(4):1013-1020; and Samanta D “PCDH19-Related Epilepsy Syndrome: A Comprehensive Clinical Review.” Pediatr Neurol. 2020; 105:3-9). GRIN2A is also involved in the pathophysiology of epilepsy in some embodiments.

Essential tremor: As used herein, the term “essential tremor” refers to a neurological condition characterized by involuntary shaking movements. It is sometimes also known as familial tremor or benign essential tremor. Essential tremor affects both men and women, and is most common in people 40 and older. Tremors are most likely to be noticed in the forearm and hands, and the upper arms, head, eyelids, and other muscles may also be affected. People with essential tremor may have trouble holding or using small objects such as silverware or writing utensils. The shaking associated with essential tremor most commonly involves small, rapid movements occurring 4 to 12 times a second. Specific symptoms may include head nodding, shaking or quivering sound to the voice (if the tremor affects the voice box), and problems with writing, drawing, drinking from a cup, or using tools (e.g., if the tremor affects the hands and/or forearms). Essential tremor typically worsens over time and can be severe in some patients. The exact cause of essential tremor is unknown, though many cases of essential tremor are genetic in origin and are inherited in an autosomal dominant manner. Mutated genes associated with essential tremor may affect various regions of the brain, including the deep brain, such as the thalamus, as well as the cerebellum.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Friedreich's ataxia: As used herein, the term “Friedreich's ataxia” refers to an autosomal recessive genetic disease caused by mutations in the FXN gene and is characterized by progressive damage of muscle tissues and the nervous system. Friedreich's ataxia is a neurological disorder associated with an expansion of a GAA trinucleotide repeat in the FXN gene that leads to a decrease in the expression of FXN. The expanded GAA trinucleotide repeat, located within the first intron, forms a R-loop which can interfere with normal transcriptional processes to reduce FXN gene expression. FXN alleles in healthy individuals contain <36 GAA repeats, whereas in FRDA patients GAA expansions ranging from 70 to 1700 GAA repeats lead to FXN mRNA deficiency and subsequent reduced levels of frataxin, a nuclear-encoded mitochondrial protein essential for life (see, e.g., Silva et al., “Expanded GAA repeats impair FXN gene expression and reposition the FXN locus to the nuclear lamina in single cells.” Hum. Molec. Genet., 2015, Vol. 24, No. 12 3457-3471). Friedreich's ataxia, the genetic basis for the disease, and related symptoms are described in the art (see, e.g., Montermini, L. et al. “The Friedreich's ataxia GAA triplet repeat: premutation and normal alleles.” Hum. Molec. Genet., 1997, 6: 1261-1266.; Filla, A. et al. “The relationship between trinucleotide (GAA) repeat length and clinical features in Friedreich's ataxia.” Am. J. Hum. Genet. 1996, 59: 554-560.; Pandolfo, M. Friedreich's ataxia: the clinical picture. J. Neurol. 2009, 256, 3-8.) Friedreich's ataxia is associated with Online Mendelian Inheritance in Man (OMIM) Entry #229300.

Frontotemporal dementia: As used herein, the term “Frontotemporal dementia” or “FTD” refers to a disease in which there is progressive degradation of the frontal and/or temporal lobes of the brain. FTD results in progressive deficits in behavior, executive function, and/or language, symptoms include: changes in social and personal behavior, apathy, blunting of emotions, deficits in expressive language, and deficits in processing language. FTD is considered one of the most prevalent forms of dementia representing 10% to 20% of all dementia cases. FTD can generally be categorized as: (i) behavioral variant FTD (bVFTD), (ii) primary progressive aphasia (PPA), (iii) progressive supranuclear palsy (PSP), and (iv) corticobasal syndrome (CBS). Many FTD cases are linked with mutations occurring in C9orf72, granulin (GRN), and MAPT. Additionally, pathologically, there are three major protein deposits found in the brains of FTD patients, TAR DNA binding protein 43 (TDP-43), fused in sarcoma (FUS) and tau. In some embodiments, a subject in need of treatment for FTD has a mutation in a GRN gene, a C9orf72 gene, and/or a MAPT gene. In some embodiments, a subject in need of treatment for FTD has progressive degradation of the frontal and/or temporal lobes of the brain. In some embodiments, a subject in need of treatment for FTD has TAR DNA binding protein 43 (TDP-43), fused in sarcoma (FUS) and/or tau deposits in the brain. In some embodiments, a subject in need of treatment for FTD has deficits in behavior, executive function, and/or language. In some embodiments, a subject in need of treatment for FTD has one or more of the following symptoms: changes in social and personal behavior, apathy, blunting of emotions, deficits in expressive language, and deficits in processing language.

FUS: As used herein, FUS refers to the gene which encodes RNA-binding protein FUS/TLS, also known as heterogeneous nuclear ribonucleoprotein P2. This protein is a subunit of a complex involved in the maturation of pre-mRNA, and also has been shown to be involved in a DNA repair response. Loss of function of the protein encoded by FUS results in increased DNA damage in neurons, and certain mutations in FUS impair the PARP-dependent DNA damage response, leading to neurodegeneration and RNA-binding protein FUS/TSL aggregate formation. Several mutations in FUS have been identified in ALS patients. See, e.g., Kwiatkowski, et al., “Mutations in the FUS/TLS Gene on Chromosome 16 Cause Familial Amyotrophic Lateral Sclerosis” Science 323(5918):1205-1205 (2009) and Vance, et al., “Mutations in FUS, an RNA Processing Protein, Cause Familial Amyotrophic Lateral Sclerosis Type 6” Science 323(5918):1208-1211 (2009). The mechanism by which FUS mutations cause ALS is not known, however it is believed that the toxicity likely results from a toxic gain of cytoplasmic function, as many ALS-linked FUS mutations are located in its nuclear localization signal, and mouse models that do not express FUS, and therefore have a complete loss of nuclear FUS localization, do not develop clear ALS-like symptoms.

FXN: As used herein, the term “FXN” refers to a gene that encodes frataxin, a protein implicated in iron homeostasis. In some embodiments, FXN may be a human (Gene ID: 2395), non-human primate (e.g., Gene ID: 737660), or rodent gene (e.g., Gene ID: 14297, Gene ID: 499335). In humans, a GAA repeat expansion in the first intron of FXN is associated with Friedreich's ataxia, a neurological disorder. In addition, multiple human transcript variants (e.g., as annotated under GenBank RefSeq Accession Numbers: NM_000144.4 and NM_181425.2) have been characterized that encode different protein isoforms.

GALC: As used herein, GALC refers to a gene encoding galactosylceramidase (also referred to as GALC and entrez:2581), which is a lysosomal protein. Galactosylceramidase degrades galactolipids involved in myelin production. In some embodiments, GALC may be human (e.g., Gene ID: 2581), non-human primate (e.g., Gene ID: 693322, Gene ID: 736519), or rodent (e.g., Gene ID: 14420, Gene ID: 314360). In humans, mutations in a GALC gene are associated with the development of Krabbe disease. In addition, multiple human transcript variants (e.g., as annotated under GenBank RefSeq Accession Numbers: NM_000153.4; NM_001201401.2; NM_001201402.2; XM_011536618.3; XM_047431198.1; XM_047431199.1) have been characterized that encode different protein isoforms.

Gaucher disease, type II and III: As used herein, the term “Gaucher Disease” or “GD”, refers to Gaucher disease types II and III, genetic disorders in which fatty substances (e.g., glucocerebroside) accumulate in cells and certain organs (e.g., spleen and liver). The buildup of these fatty substances can cause the organ to enlarge, negatively affecting organ function. If the bones are affected, it can weaken the bone, and if bone marrow is affected it can interfere with clotting. Type II Gaucher disease is a form of Gaucher disease that affects the central nervous system, spleen, liver, lungs, and bones. Type II Gaucher disease (also known as Gaucher type II and acute infantile neuronopathic Gaucher disease), develops symptoms within the first year of life. Symptoms of Gaucher type II include poor development, abnormal eye movement, hypertonia, laryngeal spasm, seizures, prolonged chest infections, enlarged spleen, and enlarged liver. Current enzyme replacement therapies for Gaucher type I and Gaucher type III are not effective in Goucher type II. Gaucher type II is a fatal disease with mortality usually within the first 2 years of life. Gaucher type III (also known as chronic neuronopathic Gaucher disease) develops during childhood. Initial symptoms of Gaucher type III are enlarged liver and spleen, poor eating, and less than normal weight gain. Other symptoms include, seizures, skeletal irregularities, eye movement disorders, cognitive problems, poor coordination, respiratory issues, and blood disorders. Both Gaucher type II and type III are neuropathic. Additionally, both are associated with mutations in the GBA gene. In some embodiments, a subject in need of treatment for Gaucher disease has a mutation in the GBA gene. In some embodiments, a subject in need of treatment for Gaucher disease has an accumulation glucocerebroside in cells and/or in organs. In some embodiments, a subject in need of treatment for Gaucher disease has an enlarged liver and/or an enlarged spleen. In some embodiments, a subject in need of treatment for Gaucher disease has one or more of the following symptoms: abnormal eye movement, hypertonia, laryngeal spasm, seizures, prolonged chest infections, enlarged spleen, enlarged liver. In some embodiments, a subject in need of treatment for Gaucher disease has one or more of the following symptoms: poor eating, less than normal weight gain, skeletal irregularities, eye movement disorders, cognitive problems, poor coordination, respiratory issues, and blood disorders.

GBA: As used herein, the term “GBA” refers to a gene (also referred to as GBA1, GCB, GLUC) encoding β-glucocerebrosidase (also referred to as acid β-glucosidase, D-glucosyl-N-acylsphingosine glucohydrolase, glucosylceramidase beta, Glucocerebrosidase or GCase), which is a lysosomal membrane protein. β-glucocerebrosidase cleaves β-glucosidic linkages. In some embodiments, GBA may be human (e.g., Gene ID: 2629), non-human primate (e.g., Gene ID: 719103), or rodent (e.g., Gene ID: 14466, Gene ID: 684536). In humans, mutations in a GBA gene are associated with the development of Gaucher Disease type II and type III. In addition, multiple human transcript variants (e.g., as annotated under GenBank RefSeq Accession Numbers: NM_000157.4; NM_001005741.3; NM_001005742.3; NM_001171811.2; NM_001171812.2) have been characterized that encode different protein isoforms.

GCH1: As used herein, GCH1 refers to the gene encoding GTP cyclohydrolase 1 (also referred to as GCH; DYT5; DYT14; DYT5a; GTPCH1; HPABH4B; GTP-CH-1), a protein that is a member of the GTP cyclohydrolase family, and which is the first and rate-limiting enzyme in tetrahydrobiopterin (BH4) biosynthesis, catalyzing the conversion of GTP into 7,8-dihydroneopterin triphosphate. In some embodiments, GCH1 may be a human (Gene ID: 2643), non-human primate (e.g., Gene ID: 695675), or rodent gene (e.g., Gene ID: 14528, Gene ID: 29244). In humans, mutations in a gene encoding GCH1 are associated with the development of hereditary dystonia. In addition, multiple human transcript variants (e.g., as annotated under GenBank RefSeq Accession Numbers: NM_001024071.2; NM_001024070.2; NM_001024024.2; NM_000161.3; XM_017021218.2; and XM_047431261.1) have been characterized that encode different protein isoforms.

GFAP: As used herein, the term “GFAP” refers to a gene encoding glial fibrillary acidic protein (also referred to as ALXDRD), a protein involved in cell communication in the CNS. In some embodiments, GFAP may be a human (Gene ID: 2670), non-human primate (e.g., Gene ID: 712941), or rodent gene (e.g., Gene ID: 14580, Gene ID: 24387). In humans, mutations in a GFAP gene are associated with Alexander disease. In addition, multiple human transcript variants (e.g., as annotated under GenBank RefSeq Accession Numbers: NM_002055.5 and NM_001131019.3) have been characterized that encode different protein isoforms.

GLB1: As used herein, the term “GLB1” refers to a gene (also known as EBP, ELNR1, or MPS4B) encoding galactosidase beta 1, a lysosomal enzyme which mediates catabolism of several molecules including GM1 ganglioside. GM1 ganglioside is an important factor in neuronal plasticity, neuronal repair, and release of neurotrophines in the brain. When mutated, GLB1 produces galactosidase beta 1 with reduced or eliminated function, leading to accumulation of GM1 ganglioside in the brain, ultimately resulting in neuronal death. GLB1 mutations are associated with GM1 gangliosidosis and Morquio syndrome B. In some embodiments, GLB1 may be a human gene (Gene ID: 2720), a non-human primate gene (Gene ID: 709355) or a rodent gene (Gene ID: 12091; Gene ID: 316033).

GM1 gangliosidosis: As used herein, the term “GM1 gangliosidosis” refers to a lysosomal storage disorder caused by deficiencies in β-galactosidase enzyme. GM1 gangliosidosis is an nervous system disorder that is inherited in an autosomal recessive pattern, and is associated with mutations in the GLB1 gene. Symptoms of GM1 gangliosidosis include cognitive impairments, developmental delays, skeletal abnormalities, seizures, motor impairments, and visual impairments. GM1 gangliosidosis is characterized by neuronal cell death and demyelination, inflammatory responses, autophagy, and mitochondrial dysfunction. The genetic basis of GM1 gangliosidosis is attributed to mutations in GLB1, of which there are 102 reported mutations. See Brunetti-Pierri, et al. “GM1 gangliosidosis: review of clinical, molecular, and therapeutic aspects” Mol Gen Metabolism 94(4): 391-396 (2008). GM1 gangliosidosis is closely related to both Tay-Sachs and to Sandhoff disease; treatments for Tay-Sachs and/or Sandhoff disease therefore may also be effective in treating GM1 gangliosidosis (and vice versa).

GNAL: As used herein, GNAL refers to the gene encoding G protein subunit alpha L (also referred to as HG1O and DYT25), a protein that is a stimulatory G protein alpha subunit which mediates odorant signaling in the olfactory epithelium. The G protein subunit alpha L protein couples dopamine type 1 receptors and adenosine A2A receptors and is widely expressed in the central nervous system. In some embodiments, GNAL may be a human (Gene ID: 2774), non-human primate (e.g., Gene ID: 102137826), or rodent gene (e.g., Gene ID: 14680, Gene ID: 24611). In humans, mutations in a gene encoding GNAL are associated with the development of hereditary dystonia. In addition, multiple human transcript variants (e.g., as annotated under GenBank RefSeq Accession Numbers: NM_182978.4; NM_001142339.3; NM_001261443.2; NM_001261444.2; NM_001369387.1; and XM_006722324.4) have been characterized that encode different protein isoforms.

GRIA1: As used herein, the term “GRIA1” refers to the gene encoding glutamate ionotropic receptor AMPA type subunit 1 (also referred to as GLUH1, GLUR1, GLURA, GluA1, and HBGR1), a protein implicated in neuronal signaling via glutamate neurotransmitters. In some embodiments, GRIA1 may be a human (Gene ID: 2890), non-human primate (e.g., Gene ID: 714117), or rodent gene (e.g., Gene ID: 14799, Gene ID: 50592). In humans, mutation in a GRIA1 gene is associated with epilepsy and seizures, as well as nociception-related phenotypes (e.g., pain disorders). In addition, multiple human transcript variants (e.g., as annotated under GenBank RefSeq Accession Numbers: NM_000827.4 and NM_001114183.2) have been characterized that encode different protein isoforms.

GRIN2A: As used herein, the term GRIN2A refers to a gene (also known as LKS; EPND; FESD; NR2A; GluN2A; NMDAR2A) encoding glutamate ionotropic receptor NMDA type subunit 2A (gluN2A), a protein that is one component of a subset of NMDA receptors. In some embodiments, GRIN2A may be a human (Gene ID: 2903), non-human primate (e.g., Gene ID: 102123126), or rodent gene (e.g., Gene ID: 14811, Gene ID: 24409). In humans, mutations in a GRIN2A gene are associated with epilepsy and seizures. Over 50 mutations in GRIN2A have been identified in patients with epilepsy. Many GRIN2A mutations lead to production of non-functional gluN2A protein or prevent the production of gluN2A protein, likely leading to a reduction in the number of functional NMDA receptors. Signaling therefore occurs more through other types of NMDA receptors that are more easily stimulated, resulting in excessive signaling in the brain. In addition, multiple human transcript variants (e.g., as annotated under GenBank RefSeq Accession Numbers: NM_000833.5; NM_001134407.3; NM_001134408.2) have been characterized that encode different protein isoforms.

GRN: As used herein, GRN refers to a gene (also known as GEP; GP88; PEPI; PGRN; CLN11; PCDGF) that encodes progranulin (also known as granulin precursor, proepithelin, and PC cell-derived growth factor), a protein which is active in many tissues throughout the body. Progranulin's function in the brain is not well understood, though it appears to play an important role in the survival of neurons. In humans, mutations in GRN are associated with frontotemporal dementia (FTD). FTD that is associated with GRN mutations (sometimes referred to as “GRN-FTD” or “GRN-related FTD”) has been suggested to involve accumulation of TAR DNA-binding protein 43 into aggregates in certain cells of the central nervous system, including neurons in the brain. These protein aggregates interfere with cell function and can lead to cell death. See, e.g., Baker, et al. “Mutations in progranulin cause tau-negative frontotemporal dementia linked to chromosome 17” Nature 442: 916-919 (2006); Cruts, et al., “Null mutations in progranulin cause ubiquitin-positive frontotemporal dementia linked to chromosome 17q21” Nature 442: 920-924 (2006); Borroni, et al. “Progranulin genetic variations in frontotemporal lobar degeneration: evidence for low mutation frequency in an Italian clinical series” Neurogenetics 9: 197-205 (2008); and Chen-Plotkin, et al. “Genetic and clinical features of progranulin-associated frontotemporal lobar degeneration” Arch. Neurol. 68: 488-497 (2011); the entire contents of each of which are herein incorporated by reference. See also Hsiung, et al. “GRN Frontotemporal Dementia” 2007 Sep. 7 [Updated 2020 Feb. 6]. In: Adam M P, et al., editors. GeneReviews® [Internet]. Seattle (WA): University of Washington, Seattle; 1993-2023. Available from: ncbi.nlm.nih.gov/books/NBK1371/. Mutations in granulins that cause deficiencies may play a role in lysosome disfunction. In some embodiments, GRN may be human (e.g., Gene ID: 2896), non-human primate (e.g., Gene ID: 454728, Gene ID: 714851), or rodent (e.g., Gene ID: 14824, Gene ID: 29143). In humans, mutations in a gene encoding GRN are associated with the development of FTD, such as a heterozygous mutation, causing inadequate production of progranulin. Human transcript variant annotated under GenBank RefSeq Accession Number NM_002087.4 has been characterized that encodes progranulin.

GYS1: As used herein, the term “GYS1” refers to a gene that encodes glycogen synthase, a protein which functions in the synthesis of glycogen. In some embodiments, GYS1 may be a human (Gene ID: 2997), non-human primate (e.g., Gene ID: 574233, Gene ID: 456196, Gene ID: 102134439), or rodent gene (e.g., Gene ID: 14936, Gene ID: 690987). In humans, expression of a mutant glycogen synthase protein (e.g., from a mutant GYS1 gene) results in decreased glycogen synthesis. In addition, multiple human transcript variants (e.g., as annotated under GenBank RefSeq Accession Numbers: NM_001161587.1 and NM_002103.5) have been characterized that encode different protein isoforms.

Hereditary dystonia: As used herein, the term “hereditary dystonia” refers to a movement disorder characterized by sustained or intermittent muscle contractions causing abnormal, often repetitive movements and/or postures. The dystonic movements are typically patterned and twisting, and may be associated with tremor. Some forms of hereditary dystonia are associated with neurodegeneration and progressively worsen over time, whereas other forms are independent of neurodegeneration and usually reach a plateau in symptoms after an initial period of worsening. Hereditary dystonia can be characterized by the body part(s) affected, and are typically classified as focal, affecting 1 body part (e.g., eyelids, mouth, larynx, neck, or hand and arm); segmental, affecting 2 or more contiguous body parts (e.g., axial—neck and trunk; brachial—1 arm and trunk or both arms+/−neck+/−trunk; or crural—1 leg and trunk or both legs+/−trunk); multifocal—2 or more non-contiguous body parts (e.g., faciobrachial—blepharospasm and hand/arm); hemidystonia—2 or more body parts (e.g., ipsilateral arm and leg); or generalized—3 or more body parts (e.g., trunk and 2 or more other sites, +/−leg involvement). A number of genes associated with hereditary dystonia include TOR1A, THAP1, ANO3, GNAL, KMT2B, GCH1, TH, SPR, TAF1, PRKRA, ATP1A3, SGCE, PNKD, PRRT2, SLC2A1, and ECHS1. Hereditary dystonia, the genetic basis for the disease, and related symptoms are described in the art (see, e.g., Klein, et al., “Hereditary Dystonia Overview” 2003 Oct. 28 (Updated 2017 Jun. 22) In: Adam, et al., editors, GeneReviews [Internet], Seattle (WA): University of Washington, Seattle, 1993-2023, NCBI Bookshelf ID: NBK1155, PMID 20301334).

HEXA: As used herein, “HEXA” refers to a gene (also referred to as TSD and hexosaminidase subunit alpha) encoding the a subunit of the enzyme β-hexosaminidase A (also referred to as hexosaminidase A), an enzyme that breaks down GM2 gangliosides and molecules containing N-acetyl hexosamines. Mutations in HEXA reduce or eliminate the activity of β-hexaosaminidase A, resulting in the accumulation of GM2 gangliosides in neuronal cells, which can result in cell death. In some embodiments, HEXA may be human (e.g., Gene ID: 3073), non-human primate (e.g., Gene ID: 698251, Gene ID: 748732), or rodent (e.g., Gene ID: 15211, Gene ID: 300757). In humans, mutations in a gene encoding HEXA are associated with the development of Tay-Sachs. In addition, multiple human transcript variants (e.g., as annotated under GenBank RefSeq Accession Numbers: NM_000520.6; NM_001318825.2) have been characterized that encode different protein isoforms.

HEXB: As used herein, “HEXB” refers to a gene (also referred to as ENC-1AS, HEL-248, and HEL-S-111) encoding the 3 subunit of the enzyme β-hexosaminidase A (also referred to as hexosaminidase A). Wildtype HEXB is the subunit of β-hexosaminidase A, which is involved in the degradation of ganglioside GM2 and other molecules. Mutations in HEXB reduce or eliminate the activity of β-hexaosaminidase A, resulting in the accumulation of GM2 gangliosides in neuronal cells, which can result in cell death. In some embodiments, HEXB may be a human gene (Gene ID: 3074), a non-human primate gene (Gene ID: 704464) or a rodent gene (Gene ID: 15212; Gene ID: 294673). In addition, multiple human transcript variants (e.g., as annotated under GenBank RefSeq Accession Numbers: NM_000521.4; NM_001292004.2) have been characterized that encode different protein isoforms. In humans, mutations in HEXB are associated with the development of Sandhoff disease.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding non-human CDR sequences. In one embodiment, humanized anti-TfR1 antibodies and antigen binding portions are provided. Such antibodies may be generated by obtaining murine anti-TfR1 monoclonal antibodies using traditional hybridoma technology followed by humanization using in vitro genetic engineering, such as those disclosed in Kasaian et al PCT publication No. WO 2005/123126 A2.

HTT: As used herein, HTT refers to the gene encoding huntingtin protein. HTT is widely expressed, and is required for normal development. The precise function of huntingtin protein encoded by HTT is not known, but it plays an important role in nerve cells, and is involved in axonal transport. HTT is expressed in many tissues throughout the body, with the highest expression levels in the brain. Huntingtin has been found to interact directly with numerous other proteins, including several involved in transcription, transport, and cell signaling. Certain mutations in HTT result in the development of Huntington's disease. HTT includes a CAG trinucleotide repeat region. CAG trinucleotide repeat expansion is associated with Huntington's disease. In some embodiments, normal subjects have two HTT alleles comprising about 10 to about 35 CAG repeats. In some embodiments, subjects with Huntington's disease, or who are expected to develop Huntington's disease, have an HTT allele comprising 40 or more CAG repeats. In some embodiments, subjects with one or two HTT alleles comprising 36 to 40 CAG repeats may or may not develop symptoms of Huntington's disease. Mutant HTT is also referred to as mHTT.

Huntington's disease: Huntington's disease is a neurological disease, characterized by degeneration of striatal neurons. It results in the progressive degeneration of nerve cells in the brain, having a wide impact on a patient's functional abilities and usually results in movement, cognitive, and psychiatric disorders. Huntington's disease affects the entire brain, however certain regions of the brain are more highly impacted than others. The striatum, which plays a key role in movement, mood, and behavioral control, is usually the portion of the brain most affected by Huntington's disease. Huntington's disease is associated with an expansion of a CAG trinucleotide repeat in HTT.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or (e.g., and) an extracellular domain, which may optionally further comprise a ligand-binding domain. In some embodiments, a cell surface receptor becomes internalized by a cell after ligand binding. In some embodiments, a ligand may be a CNS-targeting agent or a CNS-targeting antibody. In some embodiments, an internalizing cell surface receptor is a transferrin receptor.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or (e.g., and) chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad. Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

KMT2B: As used herein, KMT2B refers to the gene encoding lysine methyltransferase 2B (also referred to as HRX2; MLL2; MLL4; TRX2; WBP7; DYT28; MLL1B; MRD68; WBP-7; and CXXC10), a protein contains multiple domains including a CXXC zinc finger, three PHD zinc fingers, two FY-rich domains, and a SET domain. In some embodiments, KMT2B may be a human (Gene ID: 9757), non-human primate (e.g., Gene ID: 102115861), or rodent gene (e.g., Gene ID: 75410, Gene ID: 102550344). In humans, mutations in a gene encoding KMT2B are associated with the development of hereditary dystonia. In addition, multiple human transcript variants (e.g., as annotated under GenBank RefSeq Accession Numbers: NM_014727.3; XM_011527561.3; XM_011527562.3; XM_047439787.1; and XR_935878.3) have been characterized that encode different protein isoforms.

Krabbe disease: As used herein, the term “Krabbe disease”, “KD”, “KRD”, “globoid cell leukodystrophy”, or “galactosylceramide lipidosis” refers to a metabolic disorder in which lipids accumulate to harmful levels in different tissues throughout the body, including the nervous system, resulting in death of cells of the central nervous system including the brain. Krabbe disease is characterized by cells that have more than one nucleus (globoid cells), which results in the breaking down of the myelin coating on nerves. Krabbe disease results from a mutation in the GALC gene, which causes deficiency in the galactosylceramidase enzyme. Galactosylceramidase is an essential enzyme in myelin metabolism. Symptoms of Krabbe disease include irritability, stiff posture, delayed mental development, delayed physical development, deterioration of motor skills, muscle weakness, hypertonia, myoclonic seizures, spasticity, fever, blindness, difficulty swallowing, and deafness. Krabbe disease is most commonly found in infants (infantile form), usually beginning before the age of one. 10%-15% of Krabbe disease patients have late onset of the disease, this occurs in a juvenile form or adult form. In some embodiments, a subject has a mutation in a GALC gene. In some embodiments, a subject has an accumulation of lipids in the central nervous system (e.g., brain). In some embodiments, a subject has globoid cells in the central nervous system (e.g., brain). In some embodiments, a subject has deficiency of a galactosylceramidase enzyme. In some embodiments, a subject has one or more of the following symptoms: irritability, stiff posture, delayed mental development, delayed physical development, deterioration of motor skills, muscle weakness, hypertonia, myoclonic seizures, spasticity, fever, blindness, difficulty swallowing, and deafness.

LRRK2: As used herein, LRRK2 refers to the gene encoding dardarin protein, which is also known as leucine-rich repeat kinase 2 and PARK8. Variants of LRRK2 are associated with an increased risk of Parkinson's disease. A mutation in LRRK2 encoding a G2019S mutant of dardarin protein has been shown to cause Parkinson's disease, and is a relatively common cause of familial Parkinson's disease. This G2019S mutation results in enhanced kinase activity of the protein. Mutations in LRRK2 are the most common known cause of familial and sporadic Parkinson's disease.

LSD: As used herein, the term “LSD” refers to genes encoding lysine-specific demethylases, proteins implicated in neuronal differentiation and physiology. One example of an LSD is LSD1 (also referred to as KDM1A, AOF2, BHC110, CPRF, and KDM1) In some embodiments, an LSD may be a human (Gene ID: 23028), non-human primate (e.g., Gene ID: 718609), or rodent gene (e.g., Gene ID: 99982, Gene ID: 500569). In humans, mutation in a gene encoding a LSD is associated with neurodegeneration, such as Alzheimer's disease, tauopathy, and/or frontotemporal dementia. In addition, multiple human transcript variants of LSDs, such as LSD1 (e.g., as annotated under GenBank RefSeq Accession Numbers: NM_015013.4 and NM_001009999.3), have been characterized that encode different protein isoforms.

MAPT: As used herein, the term “MAPT” refers to the gene encoding microtubule associated protein tau (also referred to as tau, tau-40, DDPAC, FTDP-17, MAPTL, MSTD, MTBT1, MTBT2, PPND, and PPP1R103), a protein involved in the stabilization of axonal microtubules. In some embodiments, MAPT may be a human (Gene ID: 4137), non-human primate (e.g., Gene ID: 574327), or rodent gene (e.g., Gene ID: 17762, Gene ID: 29477). In humans, mutation(s) in a MAPT gene may be associated with the development of Alzheimer's disease. Mutation(s) in a MAPT gene may also be associated with certain tauopathies including frontotemporal dementia. Aggregates formed by hyperphosphorylated tau protein contribute to the pathology of Alzheimer's disease. In addition, multiple human transcript variants (e.g., as annotated under GenBank RefSeq Accession Numbers: NM_016835.5, NM_005910.6, and NM_001377265.1) have been characterized that encode different protein isoforms.

MECP2: As used herein, the term MECP2 refers to a gene (also known as RS; RTS; RTT; PPMX; MRX16; MRX79; MRXSL; AUTSX3; MRXS13) encoding methyl-CpG binding protein 2, a protein which binds to methylated DNA and has important roles in mammalian development. In some embodiments, MECP2 may be a human (Gene ID: 4204), non-human primate (e.g., Gene ID: 102135563), or rodent gene (e.g., Gene ID: 17257, Gene ID: 29386). In humans, mutation(s) in a MECP2 gene are the cause of most cases of Rett syndrome. Multiple human transcript variants (e.g., as annotated under GenBank RefSeq Accession Numbers: NM_004992.4; NM_001110792.2; NM_001316337.2; NM_001369391.2; NM_001369392.2; NM_001369393.2; NM_001369394.2; NM_001386137.1; NM_001386138.1; NM_001386139.1) have been characterized that encode different protein isoforms. Suppression of MECP2 (e.g., mutant forms thereof) may be effective in treating Rett syndrome. Increasing levels and/or activity of methyl-CpG binding protein 2 or functional fragments thereof may also be effective in treating Rett syndrome.

Metachromatic leukodystrophy (MLD): As used herein, the term “Metachromatic leukodystrophy” or “MLD” refers to a lysosomal storage disease (LSD) that is characterized by deficiency in lysosomal enzyme arylsulfatase A (ARSA) or its sphingolipid activator protein B (SapB), causing dysfunction and destruction of myelin sheaths in the central nervous system and peripheral nervous system. This leads to progressive deterioration of neurodevelopment and neurocognitive functions. Metachromatic leukodystrophy is associated with mutations in the arylsulfatase A gene (ARSA) and/or in the prosaposin gene (PSAP). Presently there is no treatment that is effective against Metachromatic leukodystrophy. In some embodiments, a subject (e.g., a subject diagnosed as having MLD) has deterioration of myelin sheaths. In some embodiments, a subject (e.g., a subject diagnosed as having MLD) has deterioration of neurodevelopment and/or neurocognitive functions.

Molecular payload: As used herein, the term “molecular payload” refers to a molecule or species that functions to modulate a biological outcome. In some embodiments, a molecular payload is linked to, or otherwise associated with a CNS-targeting agent. In some embodiments, the molecular payload is a small molecule, a polypeptide (e.g., a protein, a peptide, an antibody), a gene therapy payload (e.g., a nucleic acid), or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene. In some embodiments, the molecular payload is a polypeptide with biological activity in a particular disease context (e.g., a CNS disease or disorder). In some embodiments, the molecular payload is a small molecule with biological activity in a particular disease context (e.g., a CNS disease or disorder). In some embodiments, the molecular payload is a gene therapy payload that encodes a biologically active compound (e.g., a polypeptide).

Motor neuron disease: As used herein, the term “motor neuron disease” refers to a group of progressive neurological disorders that destroy motor neurons, which control skeletal muscle activity. Motor neuron disease includes diseases such as ALS, progressive bulbar palsy, primary lateral sclerosis, progressive muscular atrophy, spinal muscular atrophy, Kennedy's disease, and post-polio syndrome. Various genes and mutations therein are associated with the development of motor neuron disease. For example, APOE (e.g., APOE4 allele) is associated with the development of certain types of motor neuron disease. Accordingly, allele-specific modulation of APOE (e.g., APOE4) in some embodiments is useful in the treatment of motor neuron disease. In some embodiments, a subject in need of treatment for motor neuron disease has one or more symptoms associated therewith.

MSH3: As used herein, MSH3 refers to the gene encoding MutS Homolog 3 protein. The protein is involved in the mismatch repair system. MSH3 has a significant role in cancer in the suppression of tumors by repair of somatic mutations in DNA, and both loss of expression and over-expression of MSH3 can lead to oncogenic effects. Over-expression of MSH3 has been shown to decrease capacity for mismatch repair, and increased expression of MSH3 is associated with progression of Huntington's disease. See, e.g., Flower, et al. “MSH3 modifies somatic instability and disease severity in Huntington's and myotonic dystrophy type 1” Brain 142(7):1876-1888 (2019). Evidence also suggests that mutations in MSH3 may be associated with the development of spinocerebellar ataxia.

Niemann-Pick Type A: As used herein, the term “Niemann-Pick Type A” refers to Niemann-Pick Type A disease (NPA) a disease, also known as infantile neurovisceral acid sphingomyelinase deficiency, which is a fatal lysosomal neurodegenerative disorder associated with deficiencies in the activity of acid sphingomyelinase. NPA is inherited in an autosomal recessive pattern. Symptoms of NPA include developmental delay, hepatosplenomegaly, lung damage, visual abnormalities, neurodegeneration, and premature death. NPA disease is characterized by an accumulation of sphingomyelin in lysosomes, autophagy-lysosomal pathway dysfunction, and astrogliosis. The genetic cause of NPA disease has been identified as mutations in the ASM gene. See, e.g., Marin, et al. “c-Abl activation linked to autophagy-lysosomal dysfunction contributes to neurological impairment in Niemann-Pick type A disease” Front Cell Devel Biol. 10: 844297 (2022).

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkages. In some embodiments, an oligonucleotide may comprise one or more phosphorothioate linkages, which may be in the Rp or Sp stereochemical conformation.

Parkinson's disease: Parkinson's disease is a neurological disease that primarily affects the motor system, and is one form of synucleinopathy, as it is associated with an abnormal accumulation of the protein alpha-synuclein in the brain. Motor symptoms of the disease result from the death of cells of the substantia nigra region of the midbrain, which leads to a dopamine deficit. The cause of cell death is poorly understood, but involves accumulation of misfolded proteins into Lewy bodies in the neurons. At least 11 autosomal dominant and 9 autosomal recessive gene mutations have been implicated in the development of Parkinson's disease, including mutations in SNCA, LRRK2, PARK3, UCHL1, GIGYF2, HTRA2, EIF4G1, TMEM230, CHCHD2, RIC3, VPS35 (autosomal dominant); and in PRKN, PINK2, PARK7, ATP13A2, PLA2G6, FBXO7, DNAJC6, SYNJ1, and VPS13C (autosomal recessive). Mutations in SNCA and LRRK2 have been found to be risk factors for sporadic Parkinson's disease.

PCDH19: As used herein, the term “PCDH19” refers to the gene encoding protocadherin 19 (also referred to as DEE9, EFMR, and EIEE9), a protein involved in cell adhesion. In some embodiments, PCDH19 may be a human (Gene ID: 57526), non-human primate (e.g., Gene ID: 703042), or rodent gene (e.g., Gene ID: 279653, Gene ID: 317183). In humans, mutation in a gene encoding PCDH19 is associated with epilepsy and seizures. In addition, multiple human transcript variants (e.g., as annotated under GenBank RefSeq Accession Numbers: NM_020766.3 and NM_001184880.2) have been characterized that encode different protein isoforms.

PIKFYVE: As used herein, PIKFYVE refers to a gene (also known as CFD; FAB1; HEL37; PIP5K; PIP5K3; ZFYVE29) encoding Phosphatidylinositol 3-Phosphate 5-Kinase Type III protein (PIPKIII), which phosphorylates certain phosphatidylinositols (e.g., PtdIns and PtdIns3P). In some embodiments, PIKFYVE may be a human (Gene ID: 200576), non-human primate (e.g., Gene ID: 710115), or rodent gene (e.g., Gene ID: 18711, Gene ID: 316457). PIKFYVE and mutations therein have been implicated in ALS and frontotemporal dementia (FTD). Multiple human transcript variants (e.g., as annotated under GenBank RefSeq Accession Number: NM_015040.4; NM_152671.4; NM_001178000.2) have been characterized that encode different protein isoforms.

PNKD: As used herein, PNKD refers to the gene encoding PNKD metallo-beta-lactamase domain containing (also referred to as R1; MR1; PDC; DYT8; FPD1; MR-1; BRP17; MR-1S; PKND1; PNKD1; FKSG19; TAHCCP2; KIPP1184), a protein that is thought to play a role in the regulation of myofibrillogenesis. In some embodiments, PNKD may be a human (Gene ID: 25953), non-human primate (e.g., Gene ID: 101867223), or rodent gene (e.g., Gene ID: 56695, Gene ID: 100188944). In humans, mutations in a gene encoding PNKD are associated with the development of hereditary dystonia. In addition, multiple human transcript variants (e.g., as annotated under GenBank RefSeq Accession Numbers: NM_015488.5; NM_022572.4; NM_001077399.3; XM_017003771.2; and XM_017003772.2) have been characterized that encode different protein isoforms.

PRKRA: As used herein, PRKRA refers to the gene encoding protein activator of interferon induced protein kinase EIF2AK2 (also referred to as RAX; PACT; DYT16; HSD14), a protein kinase activated by double-stranded RNA which mediates the effects of interferon in response to viral infection. In some embodiments, PRKRA may be a human (Gene ID: 8575), non-human primate (e.g., Gene ID: 102116511), or rodent gene (e.g., Gene ID: 23992, Gene ID: 311130). In humans, mutations in a gene encoding PRKRA are associated with the development of hereditary dystonia. In addition, multiple human transcript variants (e.g., as annotated under GenBank RefSeq Accession Numbers: NM_003690.5; NM_001139517.1; NM_001139518.1; NM_001316362.2; XM_011512063.3; and XM_047446138.1) have been characterized that encode different protein isoforms.

PrP: As used herein, the term “PrP” refers to the gene encoding prion protein (also referred to as PRNP, PRIP, CD230, and CJD), a protein involved in neural function that can form cytotoxic prions. In some embodiments, PrP may be a human (Gene ID: 5621), non-human primate (e.g., Gene ID: 717859), or rodent gene (e.g., Gene ID: 19122, Gene ID: 24686). In humans, mutation in a PrP gene is associated with neurodegeneration. In addition, multiple human transcript variants (e.g., as annotated under GenBank RefSeq Accession Numbers: NM_000311.5 and NM_183079.4) have been characterized that encode different protein isoforms. In some embodiments, PrP is associated with small fiber neuropathy, nociception-related phenotypes, Alexander disease, Angelman Syndrome, autism-spectrum disorders, retinitis pigmentosa, isolated macular dystrophy, and/or multiple sclerosis.

PRRT2: As used herein, PRRT2 refers to the gene encoding proline rich transmembrane protein 2 (also referred to as PKC; EKD1; ICCA; BFIC2; BFIS2; DSPB3; DYT10; FICCA; IFITMD1), a transmembrane protein containing a proline-rich domain in its N-terminal half. In some embodiments, PRRT2 may be a human (Gene ID: 112476), non-human primate (e.g., Gene ID: 102124815), or rodent gene (e.g., Gene ID: 69017, Gene ID: 361651). In humans, mutations in a gene encoding PRRT2 are associated with the development of hereditary dystonia. In addition, multiple human transcript variants (e.g., as annotated under GenBank RefSeq Accession Numbers: NM_145239.3; NM_001256442.2; NM_001256443.2; XM_011545715.4; XM_017022887.3; XM_017022888.3; and XM_017022889.3) have been characterized that encode different protein isoforms.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Rett syndrome: As used herein, the term “Rett syndrome” refers to a spectrum of disorders associated with mutations in MECP2. Rett syndrome is a brain disorder that occurs almost exclusively in girls. Around 6 to 18 months of of age, subjects with Rett syndrome begin developing severe problems with language and communication, learning, coordination, and other brain functions. Early in childhood, affected subjects lose purposeful use of their hands and begin making repeated hand wringing, washing, or clapping motions. They tend to grow more slowly than other children and about 75% have microcephaly. Other signs and symptoms that can develop include breathing abnormalities, spitting or drooling, unusual eye movements such as intense staring or excessive blinking, cold hands and feet, irritability, sleep disturbances, seizures, and scoliosis. More than 99% of subjects with Rett syndrome have no family history of the disorder; many of these cases result from new mutations in MECP2. Suppression of mutant forms of MECP2, e.g., by antisense oligonucleotide therapy, may in some embodiments be effective in treating Rett syndrome or symptoms thereof. Increasing levels and/or activity of proteins or functional fragments thereof encoded by MECP2 (e.g., by delivery of gene therapy payloads) may also be effective in treating Rett syndrome or symptoms thereof.

Sandhoff disease: As used herein, the term “Sandhoff disease,” sometimes referred to as GM2 gangliosidosis, refers to a continuum of disorders that progressively destroy neurons in the central nervous system. Sandhoff disease is inherited in an autosomal recessive pattern. Symptoms of Sandhoff disease include progressive weakness, seizures, developmental deficits, neurological impairment, cognitive impairments, and premature death. Sandhoff disease is characterized by cortical, cerebellar, and spinal cord atrophy. The genetic cause of Sandhoff disease has been identified as about 30 mutations in the HEXB gene. See Xiao, et al. “Sandhoff Disease” 2022 Apr. 14, in: Adam M P, Mirzaa G M, Pagon R A, et al., editors. GeneReviews® [Internet]. Seattle (WA): University of Washington, Seattle; 1993-2023. In some embodiments, a subject in need of treatment for Sandhoff disease presents with progressive weakness. In some embodiments, progressive weakness comprises lower-extremity weakness.

In some embodiments, a subject in need of treatment for Sandhoff disease presents with seizures. In some embodiments, a subject in need of treatment for Sandhoff disease presents with developmental deficits. In some embodiments, developmental deficits comprise developmental plateauing and developmental regression. In some embodiments, a subject in need of treatment for Sandhoff disease presents with neurological impairments. In some embodiments, neurological impairments comprise loss of motor skills, exaggerated startle response, hypotonia, hyperreflexia, neuropathy, neuronopathy, atrophy, fasciculations, balance issues, tremors, dysarthria, dysphagia, and spasticity. In some embodiments, a subject in need of treatment for Sandhoff disease presents with cognitive impairments. In some embodiments, cognitive impairments comprise decreased attentiveness, cognitive decline, deficits in executive function, and deficits in memory. In some embodiments, a subject in need of treatment for Sandhoff disease does not present with hepatosplenomegaly. Sandhoff disease is very similar to Tay-Sachs; treatments for Tay-Sachs therefore may also be effective in treating Sandhoff disease (and vice versa). Sandhoff disease is also closely related to GM1 gangliosidosis, and therefore treatments for GM1 gangliosidosis may be effective in treating Sandhoff disease.

SCA1: As used herein, “SCA1” refers to spinocerebellar ataxia type 1, which is associated with CAG repeat expansions in ATXN1. See “Spinocerebellar ataxia.”

SCA2: As used herein, “SCA2” refers to spinocerebellar ataxia type 2, which is associated with CAG repeat expansions in ATXN2. See “Spinocerebellar ataxia.”

SCA3: As used herein, “SCA3” refers to spinocerebellar ataxia type 3, which is associated with CAG repeat expansions in ATXN3. See “Spinocerebellar ataxia.”

SCN1A: As used herein, the term “SCN1A” refers to the gene encoding sodium voltage-gated channel alpha subunit 1 (also referred to as DEE6, DEE6A, DEE6B, DRVT, EIEE6, FEB3, FEB3A, FHM3, GEFSP2, HBSCI, NAC1, Nav1.1, SCN1, and SMEI), a protein involved in the generation and propagation of action potentials in neurons. In some embodiments, SCN1A may be a human (Gene ID: 6323), non-human primate (e.g., Gene ID: 704086), or rodent gene (e.g., Gene ID: 20265, Gene ID: 81574). In humans, mutations in an SCN1A gene, such as a loss-of-function mutation in SCN1A, are associated with epilepsy and seizures, and with Dravet syndrome (severe myoclonic epilepsy of infancy (SMEI)). Gain-of-function mutations in SCN1A are associated with other neurological disorders, such as familial hemiplegic migraine, epileptic encephalopathy, and arthrogryposis. See, e.g., Brunklaus, et al. “The gain of function SCN1A disorder spectrum: novel epilepsy phenotypes and therapeutic implications” Brain 145(11): 3816-3831 (2022) and Ding, et al. “SCN1A Mutation—Beyond Dravet Syndrome: A Systematic Review and Narrative Synthesis” Front Neurol. 12: 743726 (2021). The most common mutations in the SCN1A gene include Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr. Mutations in SCN1A often result in reduced function of the encoded protein or no protein expression. In addition, multiple human transcript variants (e.g., as annotated under GenBank RefSeq Accession Numbers: NM_006920.6 and NM_001165963.4) have been characterized that encode different protein isoforms. In some embodiments, SCN1A, or mutant forms thereof, is associated with pain disorders.

SCN2A: As used herein, the term “SCN2A” refers to the gene encoding sodium voltage-gated channel alpha subunit 2 (also referred to as BFIC3, BFIS3, BFNIS, DEE11, EA9, EIEE11, HBA, HBSCI, HBSCII, NAC2, Nav1.2, SCN2A1, and SCN2A2), a protein involved in the generation and propagation of action potentials in neurons. In some embodiments, SCN2A may be a human (Gene ID: 6326), non-human primate (e.g., Gene ID: 703298), or rodent gene (e.g., Gene ID: 110876, Gene ID: 24766). In humans, mutations in an SCN2A gene, such as a gain-of-function mutation in SCN2A, are associated with epilepsy and seizures. Loss-of-function mutations in SCN2A are associated with other neurological disorders, including autism spectrum disorder, with or without epilepsy. See, e.g., Zeng, et al. “SCN2A-Related Epilepsy: The Phenotypic Spectrum, Treatment and Prognosis” Front Mol Neurosci. 15: 809951 (2022) doi: 10.3389/fnmol.2022.809951. In addition, multiple human transcript variants (e.g., as annotated under GenBank RefSeq Accession Numbers: NM_021007.3 and NM_001040142.2) have been characterized that encode different protein isoforms. In some embodiments, SCN2A, or mutant forms thereof, is associated with pain disorders.

SCN8A: As used herein, the term “SCN8A” refers to the gene encoding sodium voltage-gated channel alpha subunit 8 (also referred to as BFIS5, CERIII, CIAT, DEE13, EIEE13, MED, MYOCL2, NaCh6, Nav1.6, and PN4), a protein involved in the generation and propagation of action potentials in neurons. In some embodiments, SCN8A may be a human (Gene ID: 6334), non-human primate (e.g., Gene ID: 695972), or rodent gene (e.g., Gene ID: 20273, Gene ID: 29710). In humans, mutations in an SCN8A gene, such as a gain-of-function mutation in SCN8A, are associated with epilepsy and seizures. In addition, multiple human transcript variants (e.g., as annotated under GenBank RefSeq Accession Numbers: NM_014191.4 and NM_001330260.2) have been characterized that encode different protein isoforms. In some embodiments, SCN8A, or mutant forms thereof, is associated with pain disorders.

SCN9A: As used herein, the term “SCN9A” refers to a gene encoding sodium voltage-gated channel alpha subunit 9 (also referred to as ETHA, FEB3B, GEFSP7, HSAN2D, NE-NA, NENA, Nav1.7, PN1, and SFNP), a protein involved in the generation and propagation of action potentials in neurons. In some embodiments, SCN9A may be a human (Gene ID: 6335), non-human primate (e.g., Gene ID: 574119), or rodent gene (e.g., Gene ID: 20274, Gene ID: 78956). In humans, mutations in an SCN9A gene are associated with various pain disorders. In some embodiments, mutations in an SCN9A are associated with small fiber neuropathy and nociception-related phenotypes. In addition, multiple human transcript variants (e.g., as annotated under GenBank RefSeq Accession Numbers: NM_002977.3 and NM_001365536.1) have been characterized that encode different protein isoforms.

SGCE: As used herein, SGCE refers to the gene encoding sarcoglycan epsilon (also referred to as ESG; DYT11; epsilon-SG), a member of the sarcoglycan family. Sarcoglycans are transmembrane proteins that are components of the dystrophin-glycoprotein complex, which link the actin cytoskeleton to the extracellular matrix. Unlike other family members which are predominantly expressed in striated muscle, the epsilon sarcoglycan is more broadly expressed. In some embodiments, SGCE may be a human (Gene ID: 8910), non-human primate (e.g., Gene ID: 101865326), or rodent gene (e.g., Gene ID: 20392, Gene ID: 432360). In humans, mutations in a gene encoding SGCE are associated with the development of hereditary dystonia. In addition, multiple human transcript variants (e.g., as annotated under GenBank RefSeq Accession Numbers: NM_003919.3; NM_001099400.2; NM_001099401.2; NM_001301139.2; NM_001346713.2; NM_001346715.2; NM_001346717.2; NM_001346719.2; NM_001346720.2; NM_001362807.2; NM_001362808.2; and NM_001362809.2) have been characterized that encode different protein isoforms.

SLC2A1: As used herein, SLC2A1 refers to the gene encoding solute carrier family 2 member 1 (also referred to as CSE; PED; DYT9; GLUT; DYT17; DYT18; EIG12; GLUT1; HTLVR; GLUT-1; SDCHCN; GLUTIDS), a major glucose transporter in the mammalian blood-brain barrier. In some embodiments, SLC2A1 may be a human (Gene ID: 6513), non-human primate (e.g., Gene ID: 102144217), or rodent gene (e.g., Gene ID: 20525, Gene ID: 24778). In humans, mutations in a gene encoding SLC2A1 are associated with the development of hereditary dystonia. In addition, multiple human transcript variants (e.g., as annotated under GenBank RefSeq Accession Number: NM_006516.4) have been characterized that encode different protein isoforms.

SMN: As used, herein SMN refers to a gene which encodes the protein survival of motor neuron. Survival of motor neuron protein is involved in transcriptional splicing through its involvement in assembly of ribonucleoproteins that bind with pre-mRNA to form a spliceosome. A lack of survival of motor neuron protein activity results in widespread splicing defects, especially in spinal motor neurons, and degeneration of the spinal cord lower motor neurons. The survival of motor neuron protein is encoded by the genes SMN1 and SMN2, mutations in each of which are associated with spinal muscular atrophy, and both of which may be referred to as “SMN”. Molecular payloads useful in modulating SMN1 may also be useful in modulating SMN2, and vice versa, in the treatment of CNS diseases and disorders.

SNCA: As used herein, SNCA refers to the gene that encodes alpha-synuclein protein. Alpha-synuclein is a neuronal protein that regulates synaptic vesicle trafficking and subsequent neurotransmitter release, and is abundant in the brain. SNCA is primarily expressed in neural tissue (e.g., neurons), but can also be found in glial cells. Alpha-synuclein is found predominantly in presynaptic termini in both free and membrane-bound forms, with approximately 15% of the protein being membrane-bound at a given time in neurons. Alternative splicing of SNCA transcripts results in the production of at least three isoforms of alpha-synuclein. Alpha-synuclein aggregates to form insoluble fibrils in pathological conditions characterized by the presence of Lewy bodies, including Parkinson's disease. These pathological conditions are known as synucleinopathies. The aggregation mechanism of alpha-synuclein is uncertain. Several mutations in SNCA have been associated with Parkinson's disease, including mutations resulting in alpha-synuclein protein with amino acid substitutions A53T, A30P, E46K, H50Q, G51D, A18T, A29S, A53E, A53V, E57A, V15A, T72M, L8I, V15D, M127I, P117S, M5T, G93A, E83Q, and A30G. Alpha synuclein protein has been shown to interact with dopamine transporter, parkin (ligase), phospholipase D1, SNCAIP, tau protein, and beta amyloid.

SOD1: the term “SOD1” refers to the enzyme superoxide dismutase 1 and the gene which encodes it. SOD1 is an enzyme implicated in apoptosis, amyotrophic lateral sclerosis, and Parkinson's disease. The SOD1 protein is a 32 kDa homodimer which contains a binuclear Cu/Zn site in each subunit. The Cu/Zn site is responsible for destroying free superoxide radicals in the body by catalyzing disproportionation of superoxide to hydrogen peroxide and dioxygen. Wild-type SOD1 protein has demonstrated antiapoptotic properties in neural cultures, whereas mutant SOD1 protein has been shown to promote apoptosis in neural cells. Mutations in the SOD1 gene have been linked to familial ALS, though wild-type SOD1 has also been implicated in a significant fraction of sporadic ALS cases, which represent 90% of ALS patients. The most frequent SOD1 mutations are A4V, H46R, and G93S. Virtually all known ALS-associated SOD1 mutations act in a dominant fashion, such that a single mutant copy of the SOD1 gene is sufficient to cause the disease. The exact mechanism by which mutations in SOD1 cause ALS is unknown, though some evidence suggests that it is the result of a toxic gain of function, as many disease-associated SOD1 mutations (including A4V and G93A) retain enzymatic activity and Sod1 deficient mice do not develop ALS. The DNA oxidation product 8-OHdG, a well-established marker of oxidative DNA damage, accumulates in the mitochondria of motor neurons of ALS patients, suggesting that oxidative damage to DNA (e.g., mitochondrial DNA) of motor neurons resulting from mutated SOD1 may significantly contribute to the etiology of ALS.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., CNS cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Spinocerebellar ataxia: As used herein, the term “spinocerebellar ataxia” or “SCA” refers to a class of CNS disorders that are generally characterized by problems with coordination due to effects on the cerebellum and spinal cord (also referred to as “autosomal dominant cerebellar ataxias”). SCA is often characterized by slowly progressive incoordination of gait, and is often associated with poor coordination of hands, speech, and eye movements. SCA is a progressive neurodegenerative disorder which is inherited in an autosomal dominant pattern. More than 40 types of SCA, each of which has similar causes and symptoms. The most common form of SCA is SCA3, also known as Machado-Joseph disease. Most genetic mutations associated with SCA result in prominent damage to cerebellar Purkinje neurons with consecutive cerebellar atrophy. In addition, other parts of the CNS, such as the spinal cord, basal ganglia and pontine nuclei in the brainstem, can be involved. See, e.g., Klockgether, et al. “Spinocerebellar ataxia” Nat Rev Dis Primers 5:24 (2019) doi:10.1038/s41572-019-0074-3 and Bhandari, et al. “Spinocerebellar Ataxia.” [Updated 2022 Aug. 10]. In: StatPearls [Internet]. Treasure Island (FL): StatPearls Publishing; Available from: ncbi.nlm.nih.gov/books/NBK557816/. Of particular relevance to the present disclosure are SCA1, SCA2, SCA3, and SCA associated with MSH3. Many forms of SCA are associated with expansions of trinucleotide repeats within ataxin (ATXN) genes. SCA1 is associated with CAG repeat expansions in ATXN1, SCA2 is associated with CAG repeat expansions in ATXN2, and SCA3 is associated with CAG repeat expansions in ATXN3. There are currently no treatments available for SCA; as such, clinical interventions focus primarily on management of symptoms through physical therapy, occupational therapy, and speech therapy.

SPR: As used herein, SPR refers to the gene encoding sepiapterin reductase (also referred to as SDR38C1), an aldo-keto reductase that catalyzes the NADPH-dependent reduction of pteridine derivatives. SPR is important in the biosynthesis of tetrahydrobiopterin (BH4). Mutations in this gene result in DOPA-responsive dystonia due to sepiapterin reductase deficiency. In some embodiments, SPR may be a human (Gene ID: 6697), non-human primate (e.g., Gene ID: 102128831), or rodent gene (e.g., Gene ID: 20751, Gene ID: 29270). In humans, mutations in a gene encoding SPR are associated with the development of hereditary dystonia. In addition, multiple human transcript variants (e.g., as annotated under GenBank RefSeq Accession Number: NM_003124.5) have been characterized that encode different protein isoforms.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a CNS disease or disorder. In some embodiments, the subject is a human patient who has one or more symptoms associated with a CNS disease or disorder, such as one or more symptoms disclosed herein.

SYF2: As used herein, SYF2 refers to the gene (also known as P29; CBPIN; NTC31; fSAP29) encoding pre-mRNA-splicing factor SYF2, which is primarily localized in the nucleus. In some embodiments, SYF2 may be a human (Gene ID: 25949), non-human primate (e.g., Gene ID: 102139055), or rodent gene (e.g., Gene ID: 68592, Gene ID: 170933). SYF2 and mutations therein have been implicated in ALS and frontotemporal dementia (FTD). Multiple human transcript variants (e.g., as annotated under GenBank RefSeq Accession Number: NM_207170.4; NM_015484.5) have been characterized that encode different protein isoforms.

TAF1: As used herein, TAF1 refers to the gene encoding TATA-box binding protein associated factor 1 (also referred to as OF; XDP; BA2R; CCG1; CCGS; DYT3; KAT4; P250; NSCL2; TAF2A; MRXS33; N-TAF1; TAFII250; DYT3/TAF1; TAFII-250; TAF(II)250), a member of a group of evolutionarily conserved proteins known as TBP-associated factors. TAF1 encodes the largest subunit of the basal transcription factor TFIID, which subunit binds to core promoter sequences encompassing the transcription start site, and also binds to activators and other transcriptional regulators. In some embodiments, TAF1 may be a human (Gene ID: 6872), non-human primate (e.g., Gene ID: 102118965), or rodent gene (e.g., Gene ID: 270627, Gene ID: 317256). In humans, mutations in a gene encoding TAF1 are associated with the development of hereditary dystonia. In addition, multiple human transcript variants (e.g., as annotated under GenBank RefSeq Accession Numbers: NM_004606.5; NM_138923.4; NM_001286074.2; NR_104387.2; NR_104388.2; NR_104389.2; NR_104390.2; NR_104391.2; NR_104392.2; NR_104393.2; NR_104394.2; NR_104395.2; NR_104396.2; XM_005262300.3; XM_024452430.2; XM_047442391.1; XM_047442392.1; XM_047442393.1; XM_047442394.1; XM_047442395.1; XM_047442396.1; XM_047442397.1; XM_047442398.1; XM_047442399.1; XM_047442400.1; XM_047442401.1; XM_047442402.1; XM_047442403.1; XM_047442404.1; XM_047442405.1; and XM_047442406.1) have been characterized that encode different protein isoforms.

Tay-Sachs: As used herein, the term “Tay-Sachs” refers to a genetic disorder that is characterized by destruction of nerve cells in the central nervous system. Tay-Sachs is also known as GM2 gangliosidosis. Tay-Sachs is associated with a mutation in the enzyme hexosaminidase A (HEXA), which leads to a buildup of GM2 ganglioside in lysosomes and nerve cells. Tay-Sachs predominantly affects young children (infantile form) but can come on during adolescence (juvenile form), as well as in adulthood. Tay-Sachs is characterized by neurodegeneration, and its symptoms include: slowing of development, progressive loss of mental ability, dementia, blindness, increase startle reflex to noise, progressive loss of hearing, swallowing issues, seizures, Cherry-red spots in the eyes, muscle weakness, and ataxia. Conventional treatments for Tay-Sachs focus on symptom relief and delay in progression. In some embodiments, a subject in need of treatment for Tay-Sachs has a mutation in a HEXA gene. In some embodiments, a subject in need of treatment for Tay-Sachs has neurodegeneration. In some embodiments, a subject in need of treatment for Tay-Sachs has one or more of the following symptoms: slowing of development, progressive loss of mental ability, dementia, blindness, increase startle reflex to noise, progressive loss of hearing, swallowing issues, seizures, Cherry-red spots in the eyes, muscle weakness, and ataxia. Tay-Sachs is very similar to Sandhoff disease; treatments for Sandhoff disease therefore may also be effective in treating Tay-Sachs (and vice versa). Tay-Sachs is also closely related to GM1 gangliosidosis, and therefore treatments for GM1 gangliosidosis may be effective in treating Tay-Sachs (and vice versa).

TH: As used herein, TH refers to the gene encoding tyrosine hydroxylase (also referred to as TYH; DYT14; DYT5b), a protein involved in the conversion of tyrosine to dopamine. The tyrosine hydroxylase protein is the rate-limiting enzyme in the synthesis of catecholamines, and hence plays a key role in the physiology of adrenergic neurons. In some embodiments, TH may be a human (Gene ID: 7054), non-human primate (e.g., Gene ID: 102134074), or rodent gene (e.g., Gene ID: 21823, Gene ID: 25085). In humans, mutations in a gene encoding TH are associated with the development of hereditary dystonia. In addition, multiple human transcript variants (e.g., as annotated under GenBank RefSeq Accession Numbers: NM_000360.4; NM_199292.3; NM_199293.3; and XM_011520335.3) have been characterized that encode different protein isoforms.

THAP1: As used herein, THAP1 refers to the gene encoding THAP domain containing 1 (also referred to as DYT6), a protein that contains a THAP domain, a conserved DNA-binding domain. This protein colocalizes with the apoptosis response protein PAWR/PAR-4 in promyelocytic leukemia (PML) nuclear bodies, and functions as a proapoptotic factor that links PAWR to PML nuclear bodies. In some embodiments, THAP1 may be a human (Gene ID: 55145), non-human primate (e.g., Gene ID: 101926823), or rodent gene (e.g., Gene ID: 73754, Gene ID: 306547). In humans, mutations in a gene encoding THAP1 are associated with the development of hereditary dystonia. In addition, multiple human transcript variants (e.g., as annotated under GenBank RefSeq Accession Numbers: NM_018105.3 and NM_199003.2) have been characterized that encode different protein isoforms.

TOR1A: As used herein, TOR1A refers to the gene encoding torsin family 1 member A (also referred to as DQ2; AMC5; DYT1), a is a member of the AAA family of adenosine triphosphatases (ATPases). In some embodiments, TOR1A may be a human (Gene ID: 1861), non-human primate (e.g., Gene ID: 102124758), or rodent gene (e.g., Gene ID: 30931, Gene ID: 266606). In humans, mutations in a gene encoding TOR1A are associated with the development of hereditary dystonia. In addition, multiple human transcript variants (e.g., as annotated under GenBank RefSeq Accession Number: NM_000113.3) have been characterized that encode different protein isoforms.

TPP1: As used herein, “TPP1” refers to the gene encoding tripeptidyl peptidase 1 (also known as GIG1, LPIC, SCAR7, and CLN2). The tripeptidyl peptidase 1 enzyme is implicated in CLN2 Batten disease. Wildtype tripeptidyl peptidase 1 mediates cleavage of N-terminal tripeptides from substrates. In CLN2 Batten disease, mutations in tripeptidyl peptidase 1 enzymes severely decrease its enzymatic activity, leading to the incomplete breakdown, and subsequent accumulation of proteins in lysosomes. The most frequent tripeptidyl peptidase 1 mutations are single amino acid changes. Inheritance of CLN2 Batten disease is autosomal recessive. In some embodiments, CLN2 may be a human (Gene ID: 1200), non-human primate (Gene ID: 709838), or rodent (Gene ID: 12751; Gene ID: 83534) gene.

Transferrin receptor: As used herein, the term, “transferrin receptor” (also known as TFRC, CD71, p90, TFR, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

TREM2: As used herein, TREM2 refers to the gene which encodes triggering receptor expressed on myeloid cells 2 (also referred to as PLOSL2, Trem2a, Trem2b, and Trem2c), a protein involved in inflammation, synaptic pruning, and neuronal cell survival. In the brain, TREM2 is expressed in microglial cells. In some embodiments, TREM2 may be a human (Gene ID: 54209), non-human primate (e.g., Gene ID: 719740), or rodent gene (e.g., Gene ID: 83433, Gene ID: 301227). In humans, mutation in a TREM2 gene is associated with the development of Alzheimer's disease. In some embodiments, mutations in TREM2 are associated with an increased risk of Alzheimer's disease. Genetic variants of TREM2 have also been associated with increased risk of multiple neurodegenerative disease, including frontotemporal dementia and Alzheimer's disease. See, e.g., Carmona, et al. “The role of TREM2 in Alzheimer's disease and other neurodegenerative disorders” Lancet Neurology 17(8):721-730 (2018). In addition, multiple human transcript variants (e.g., as annotated under GenBank RefSeq Accession Numbers: NM_018965.4 and NM_001271821.2) have been characterized that encode different protein isoforms.

UBE3A: As used herein, the term “UBE3A” refers to a gene encoding ubiquitin protein ligase E3A (also referred to as E6AP, ANCR, AS, EPVE6AP, HPVE6A, and PIX1), a protein implicated in ubiquitination and proteolysis. In some embodiments, UBE3A may be a human (Gene ID: 7337), non-human primate (e.g., Gene ID: 711270), or rodent gene (e.g., Gene ID: 22215, Gene ID: 361585). In humans, mutation in a UBE3A gene is associated with Angelman Syndrome and autism-spectrum disorders. In addition, multiple human transcript variants (e.g., as annotated under GenBank RefSeq Accession Numbers: NM_130838.4, NM_000462.5, and NM_130839.5) have been characterized that encode different protein isoforms.

UNC13A: As used herein, the term “UNC13A” refers to a gene encoding Unc-13 homolog A (also referred to as Munc13-1 and unc-13 homolog A (C. elegans)), a member of the UNC13 family of proteins, which are involved in calcium-triggered synaptic vesicle release (See, e.g., J. S. Dittman “Unc13: a multifunctional synaptic marvel” Curr Opin Neurobiol. 57:17-25 (2019)). In some embodiments, UNC13A may be a human (Gene ID: 23025), non-human primate (e.g., Gene ID: 720000, Gene ID: 102123626), or rodent gene (e.g., Gene ID: 382018, Gene ID: 64829). In addition, multiple human transcript variants (e.g., as annotated under GenBank RefSeq Accession Numbers: NM_001080421.3, NM_001387021.1, NM_001387022.1, NM_001387023.1, XM_011527810.3, XM_011527811.3, XM_017026502.2, XM_054320277.1, XM_054320278.1, and XM_054320279.1) have been characterized that encode different protein isoforms. UNC13A contains a cryptic exon that promotes nonsense-mediated decay. Certain single nucleotide polymorphisms in UNC13A are associated with an increased risk of cryptic exon inclusion in the UNC13A transcript. Such polymorphisms in UNC13A have been associated with neurodegenerative diseases such as ALS and frontotemporal dementia (See, e.g., Brown, et al. “TDP-43 loss and ALS-risk SNPs drive mis-splicing and depletion of UNC13A” Nature 603:131-137 (2022); and Ma, et al. “TDP-43 represses cryptic exon inclusion in the FTD-ALS gene UNC13A” Nature 603:124-130 (2022)).

VLA-4: As used herein, the term “VLA-4” refers to a gene encoding very late antigen 4 (also referred to as ITGA4, CD49D, and IA4), a protein involved in cell adhesion and signaling. In some embodiments, VLA-4 may be a human (Gene ID: 3676), non-human primate (e.g., Gene ID: 704745), or rodent gene (e.g., Gene ID: 16401, Gene ID: 311144). In humans, mutations in a VLA-4 gene are associated with retinitis pigmentosa 26, isolated macular dystrophy, and multiple sclerosis. In addition, multiple human transcript variants (e.g., as annotated under GenBank RefSeq Accession Numbers: NM_000885.6 and NM_001316312.2) have been characterized that encode different protein isoforms.

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleosides and oligonucleotides comprising the 2′-modified nucleosides have increased affinity to a target sequences, relative to an unmodified oligonucleotide. Examples of structures of 2′-modified nucleosides are provided below:

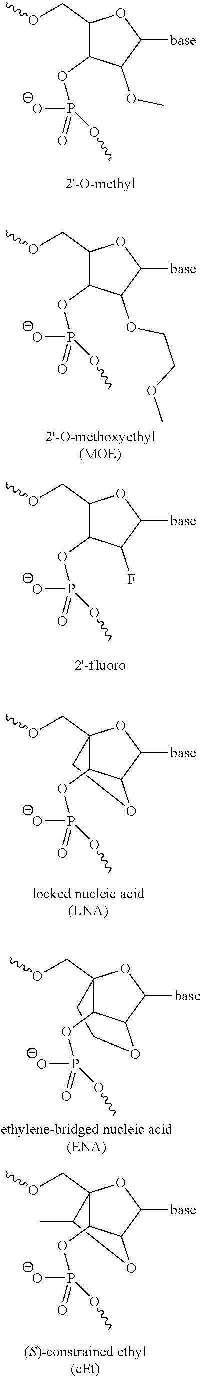

These examples are shown with phosphate groups, but any internucleoside linkages are contemplated between 2′-modified nucleosides.

### II. Complexes

Provided herein are complexes that comprise a targeting agent, e.g. an antibody, covalently linked to a molecular payload. In some embodiments, a complex comprises a central nervous system (CNS)-targeting antibody covalently linked to an oligonucleotide. A complex may comprise an antibody that specifically binds a single antigenic site or that binds to at least two antigenic sites that may exist on the same or different antigens.

A complex may be used to modulate the activity or function of at least one gene, protein, and/or (e.g., and) nucleic acid in cells of the CNS, or to alleviate the symptoms of a CNS disease disorder. In some embodiments, the molecular payload present with a complex is responsible for the modulation of a gene, protein, and/or (e.g., and) nucleic acids. A molecular payload may be a small molecule, protein, nucleic acid, oligonucleotide, or any molecular entity capable of modulating the activity or function of a gene, protein, and/or (e.g., and) nucleic acid in a cell.

In some embodiments, a CNS targeting agent of the complexes described herein comprises an anti-transferrin receptor 1 (TfR1) antibody, covalently linked to a molecular payload, e.g., an oligonucleotide, polypeptide, small molecule, or gene therapy payload.

### Antibodies

In some embodiments, complexes described herein comprise an antibody that binds human transferrin receptor 1 (TfR1). An example human TfR1 amino acid sequence, corresponding to NCBI sequence NP_003225.2 (transferrin receptor protein 1 isoform 1, Homo sapiens) is as follows:

(SEQ ID NO: 21)
MMDQARSAFSNLFGGEPLSYTRFSLARQVDGDNSHVEMKLAVDEEEN
ADNNTKANVTKPKRCSGSICYGTIAVIVFFLIGFMIGYLGYCKGVEP
KTECERLAGTESPVREEPGEDFPAARRLYWDDLKRKLSEKLDSTDFT
GTIKLLNENSYVPREAGSQKDENLALYVENQFREFKLSKVWRDQHFV
KIQVKDSAQNSVIIVDKNGRLVYLVENPGGYVAYSKAATVTGKLVHA
NFGTKKDFEDLYTPVNGSIVIVRAGKITFAEKVANAESLNAIGVLIY
MDQTKFPIVNAELSFFGHAHLGTGDPYTPGFPSFNHTQFPPSRSSGL
PNIPVQTISRAAAEKLFGNMEGDCPSDWKTDSTCRMVTSESKNVKLT
VSNVLKEIKILNIFGVIKGFVEPDHYVVVGAQRDAWGPGAAKSGVGT
ALLLKLAQMFSDMVLKDGFQPSRSIIFASWSAGDFGSVGATEWLEGY
LSSLHLKAFTYINLDKAVLGTSNFKVSASPLLYTLIEKTMQNVKHPV
TGQFLYQDSNWASKVEKLTLDNAAFPFLAYSGIPAVSFCFCEDTDYP
YLGTTMDTYKELIERIPELNKVARAAAEVAGQFVIKLTHDVELNLDY
ERYNSQLLSFVRDLNQYRADIKEMGLSLQWLYSARGDFFRATSRLTT
DFGNAEKTDRFVMKKLNDRVMRVEYHFLSPYVSPKESPFRHVFWGSG
SHTLPALLENLKLRKQNNGAFNETLFRNQLALATWTIQGAANALSGD
VWDIDNEF.

Table 2 provides examples of sequences of an anti-TfR1 antibody useful in the complexes provided herein.

TABLE 2
Examples of anti-TfR1 antibody sequences

| Feature | IMGT | Kabat | Chothia |
|---|---|---|---|
| CDR-H1 | GYSITSGYY | SGYYWN | GYSITSGY |
| | (SEQ ID NO: 1) | (SEQ ID NO: 7) | (SEQ ID NO: 12) |
| CDR-H2 | ITFDGAN | YITFDGANNYNPSL | FDG |
| | (SEQ ID NO: 2) | KN | (SEQ ID NO: 13) |
| | | (SEQ ID NO: 8) | |
| CDR-H3 | TRSSYDYDVLDY | SSYDYDVLDY | SYDYDVLD |
| | (SEQ ID NO: 3) | (SEQ ID NO: 9) | (SEQ ID NO: 14) |
| CDR-L1 | QDISNF | RASQDISNFLN | SQDISNF |
| | (SEQ ID NO: 4) | (SEQ ID NO: 10) | (SEQ ID NO: 15) |
| CDR-L2 | YTS | YTSRLHS | YTS |
| | (SEQ ID NO: 5) | (SEQ ID NO: 11) | (SEQ ID NO: 5) |
| CDR-L3 | QQGHTLPYT | QQGHTLPYT | GHTLPY |
| | (SEQ ID NO: 6) | (SEQ ID NO: 6) | (SEQ ID NO: 16) |

| | |
|---|---|
| VH | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSS (SEQ ID NO: 17) |
| VL | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTF |
| | GQGTKLEIK (SEQ ID NO: 18) |
| Fab HC | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSSASTKGPSVFPLAPSSKSTSGGTAALGCL |
| | VKDYFPEPVTVSWNSGALTSGVHTFPAVLQSSGLYSLSSVVTVPSSSLG |
| | TQTYICNVNHKPSNTKVDKKVEPKSCDKTHT (SEQ ID NO: 19) |
| Fab LC | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTF |
| | GQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACEV |
| | THQGLSSPVTKSFNRGEC (SEQ ID NO: 20) |

In some embodiments, the anti-TfR1 antibody of the present disclosure comprises a heavy chain complementarity determining region 1 (CDR-H1) of SEQ ID NO: 1 (according to the IMGT definition system), a heavy chain complementarity determining region 2 (CDR-H2) of SEQ ID NO: 2 (according to the IMGT definition system), a heavy chain complementarity determining region 3 (CDR-H3) of SEQ ID NO: 3 (according to the IMGT definition system), a light chain complementarity determining region 1 (CDR-L1) of SEQ ID NO: 4 (according to the IMGT definition system), a light chain complementarity determining region 2 (CDR-L2) of SEQ ID NO: 5 (according to the IMGT definition system), and a light chain complementarity determining region 3 (CDR-L3) of SEQ ID NO: 6 (according to the IMGT definition system).

In some embodiments, the anti-TfR1 antibody of the present disclosure comprises a heavy chain complementarity determining region 1 (CDR-H1) of SEQ ID NO: 7 (according to the Kabat definition system), a heavy chain complementarity determining region 2 (CDR-H2) of SEQ ID NO: 8 (according to the Kabat definition system), a heavy chain complementarity determining region 3 (CDR-H3) of SEQ ID NO: 9 (according to the Kabat definition system), a light chain complementarity determining region 1 (CDR-L1) of SEQ ID NO: 10 (according to the Kabat definition system), a light chain complementarity determining region 2 (CDR-L2) of SEQ ID NO: 11 (according to the Kabat definition system), and a light chain complementarity determining region 3 (CDR-L3) of SEQ ID NO: 6 (according to the Kabat definition system).

In some embodiments, the anti-TfR1 antibody of the present disclosure comprises a heavy chain complementarity determining region 1 (CDR-H1) of SEQ ID NO: 12 (according to the Chothia definition system), a heavy chain complementarity determining region 2 (CDR-H2) of SEQ ID NO: 13 (according to the Chothia definition system), a heavy chain complementarity determining region 3 (CDR-H3) of SEQ ID NO: 14 (according to the Chothia definition system), a light chain complementarity determining region 1 (CDR-L1) of SEQ ID NO: 15 (according to the Chothia definition system), a light chain complementarity determining region 2 (CDR-L2) of SEQ ID NO: 5 (according to the Chothia definition system), and a light chain complementarity determining region 3 (CDR-L3) of SEQ ID NO: 16 (according to the Chothia definition system).

In some embodiments, the anti-TfR1 antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 17. Alternatively or in addition (e.g., in addition), the anti-TfR1 antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 18.

In some embodiments, the anti-TfR1 antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 17. Alternatively or in addition (e.g., in addition), in some embodiments, the anti-TfR1 antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 18.

In some embodiments, the anti-TfR1 antibody of the present disclosure comprises a VH comprising the amino acid sequence of SEQ ID NO: 17. Alternatively or in addition (e.g., in addition), in some embodiments, the anti-TfR1 antibody of the present disclosure comprises a VL comprising the amino acid sequence of SEQ ID NO: 18.

In some embodiments, the anti-TfR1 antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure comprises a light chain comprising an amino acid sequence least 75% (e.g., 75%, 80%, 85%, 90%, 95%, 98%, or 99%) identical to the amino acid sequence of SEQ ID NO: 20. In some embodiments, the anti-TfR1 antibody of the present disclosure is a Fab that comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure is a Fab that comprises a light chain comprising an amino acid sequence least 75% (e.g., 75%, 80%, 85%, 90%, 95%, 98%, or 99%) identical to the amino acid sequence of SEQ ID NO: 20.

In some embodiments, the anti-TfR1 antibody of the present disclosure comprises a heavy chain comprising the amino acid sequence of SEQ ID NO: 19. Alternatively or in addition (e.g., in addition), the anti-TfR1 antibody of the present disclosure comprises a light chain comprising the amino acid sequence of SEQ ID NO: 20. In some embodiments, the anti-TfR1 antibody of the present disclosure is a Fab that comprises a heavy chain comprising an amino acid sequence of SEQ ID NO: 19. Alternatively or in addition (e.g., in addition), the anti-TfR1 antibody of the present disclosure is a Fab that comprises a light chain comprising the amino acid sequence of SEQ ID NO: 20.

In some embodiments, the anti-TfR1 antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

### Molecular Payloads

Some aspects of the disclosure provide molecular payloads, e.g., for modulating a biological outcome, e.g., the transcription of a DNA sequence, the processing of a pre-mRNA transcript, the stability of a pre-mRNA or mRNA transcript, the expression of a protein (e.g., translation of an mRNA), or the activity of a protein, that can be linked to an anti-TfR1 antibody described herein (e.g., anti-TfR1 antibody in Table 2). In some embodiments, such molecular payloads are targeted to a CNS cell, e.g., via specifically binding to a nucleic acid or protein in or on the CNS cell following delivery to the CNS cell by the linked anti-TfR1 antibody. It should be appreciated that various types of molecular payloads may be used in accordance with the disclosure. For example, the molecular payload may comprise, or consist of, an oligonucleotide (e.g., an antisense oligonucleotide or an RNA interference oligonucleotide), a polypeptide (e.g., a peptide, protein, or antibody that binds a nucleic acid or protein in a CNS cell), a small molecule (e.g., a small molecule that modulates the function of a nucleic acid or protein in a CNS cell), or a gene therapy payload (e.g., a nucleic acid that encodes a polypeptide with biological activity in a CNS cell).

In some embodiments, the molecular payload is an oligonucleotide that comprises a strand having a region of complementarity to a gene (e.g., a gene transcript) provided in Table 3.

TABLE 3
List of central nervous system diseases and corresponding genes.

| | Gene | | SEQ ID |
|---|---|---|---|
| Disease | Symbol | Transcripts GenBank Accession No. | NO: |
| Amyotrophic lateral | SOD1 | NM_000454.5 | 392 |
| sclerosis (ALS) | C9orf72 | NM_001256054.3; NM_018325.5; | 393-395 |
| | | NM_145005.7 | |
| | ATXN2 | NM_001310121.1; NM_001310123.1; | 396-400 |
| | | NM_001372574.1; NM_002973.4; | |
| | | NR_132311.2 | |
| | FUS | NM_001170634.1; NM_001170937.1; | 401-404 |
| | | NM_004960.4; NR_028388.2 | |
| | PIKFYVE | NM_015040.4; NM_152671.4; | 143-148 |
| | | NM_001178000.2 | |
| | SYF2 | NM_207170.4; NM_015484.5 | 167-168 |
| | UNC13A | NM_001080421.3; NM_001387021.1; | 169, |
| | | NM_001387022.1; NM_001387023.1; | 810-818 |
| | | XM_011527810.3; XM_011527811.3; | |
| | | XM_017026502.2; XM_054320277.1; | |
| | | XM_054320278.1; XM_054320279.1 | |
| Spinocerebellar | ATXN1 | NM_000332.4; NM_001128164.2; | 819; 820; |
| ataxia | | NM_001357857.2 | 819 |
| | ATXN2 | See above | 396-400 |
| | ATXN3 | NM_004993.6; NM_030660.5; | 821-832 |
| | | NM_001127696.2; NM_001127697.3; | |
| | | NM_001164774.2; NM_001164776.2; | |
| | | NM_001164777.2; NM_001164778.2; | |
| | | NM_001164779.2; NM_001164780.2; | |
| | | NM_001164781.2; NM_001164782.2 | |
| | MSH3 | NM_002439.5 | 437 |
| Parkinson's | LRRK2 | XR_007063041.1; NM_198578.4; | 405-416 |
| disease | | XM_005268629.5; XM_011537877.4; | |
| | | XM_047428277.1; XM_047428278.1; | |
| | | XM_011537881.4; XM_047428279.1; | |
| | | XM_017018786.3; XM_011537882.4; | |
| | | XM_024448833.2; XM_017018787.2 | |
| | SNCA | NR_164674.1; NR_164675.1; | 417-434 |
| | | NR_164676.1; NM_001146055.2; | |
| | | NM_001375285.1; NM_001375286.1; | |
| | | NM_000345.4; NM_007308.3; | |
| | | NM_001146054.2; NM_001375287.1; | |
| | | NM_001375288.1; NM_001375290.1; | |
| | | XM_011532205.3; XM_011532206.2; | |
| | | XM_011532203.2; XM_011532204.4; | |
| | | XM_047416097.1; XM_011532207.2 | |
| Huntington's | HTT | NM_002111.8; NM_001388492.1 | 435-436 |
| disease | MSH3 | NM_002439.5 | 437 |
| Neuromuscular | DMPK | NM_001081563.2; NM_001288766.2; | 438-444 |
| disease | | NM_001081562.3; NM_001081560.3; | |
| | | NM_004409.5; NM_001288764.2; | |
| | | NM_001288765.2 | |
| | DMD | XM_006724469.4; NM_000109.4; | 445-474 |
| | | XM_011545467.2; XM_006724473.3; | |
| | | XM_006724474.4; XM_006724475.3; | |
| | | XM_006724470.4; XM_017029328.2; | |
| | | XM_006724468.3; NM_004006.3; | |
| | | NM_004011.4; NM_004012.4; | |
| | | NM_004023.3; NM_004020.4; | |
| | | NM_004022.3; NM_004021.3; | |
| | | NM_004013.3; NM_004014.3; | |
| | | NM_004018.3; NM_004017.3; | |
| | | NM_004016.3; NM_004015.3; | |
| | | NM_004009.3; NM_004010.3; | |
| | | NM_004019.3; XM_011545468.3; | |
| | | XM_017029330.3; XM_017029329.2; | |
| | | XM_047441889.1; XM_011545469.2 | |
| | SMN1 | XM_047417615.1; XM_017009786.2; | 475-496 |
| | | XM_011543597.2; XM_047417618.1; | |
| | | NM_001297715.1; NM_022874.2; | |
| | | XM_047417616.1; XM_011543596.2; | |
| | | NM_000344.4; XM_047443291.1; | |
| | | XM_047443295.1; XM_047443301.1; | |
| | | XM_047443298.1; XM_047443297.1; | |
| | | XM_047443294.1; XM_047443303.1; | |
| | | XM_047443300.1; XM_047443296.1; | |
| | | XM_047443292.1; XM_047443302.1; | |
| | | XM_047443299.1; XM_047443293.1 | |
| | SMN2 | NM_017411.4; NM_022875.3; | 833-836 |
| | | NM_022876.2; NM_022877.2 | |
| | FXN | NM_181425.3; NM_000144.5 | 497-498 |
| Alzheimer's | TREM2 | NM_018965.4; NM_001271821.2 | 499-500 |
| disease | APOE | NM_000041.4; NM_001302688.2; | 501-505 |
| | | NM_001302689.2; NM_001302690.2; | |
| | | NM_001302691.2 | |
| | MAPT | NR_165166.1; NM_001377268.1; | 506-534 |
| | | XM_005257362.5; XM_005257365.5; | |
| | | XM_005257367.5; XM_005257368.5; | |
| | | NM_001123066.4; NM_016835.5; | |
| | | XM_005257369.5; XM_047436079.1; | |
| | | NM_005910.6; NM_001203252.2; | |
| | | NM_001377265.1; XM_047436074.1; | |
| | | XM_047436076.1; NM_001377266.1; | |
| | | XM_005257370.5; XM_047436080.1; | |
| | | NM_001123067.4; NM_001203251.2; | |
| | | NM_001377267.1; XM_005257366.4; | |
| | | XM_047436075.1; XM_047436077.1; | |
| | | XM_047436078.1; XM_005257371.5; | |
| | | XM_047436081.1; NM_016834.5; | |
| | | NM_016841.5 | |
| | APP | NM_001136131.3; NM_001136129.3; | 535-545 |
| | | NM_001136130.3; NM_001204303.2; | |
| | | NM_201414.3; NM_001385253.1; | |
| | | NM_001204302.2; NM_201413.3; | |
| | | NM_001204301.2; NM_000484.4; | |
| | | NM_001136016.3 | |
| Frontotemporal | GRN | NM_002087.4 | 837 |
| dementia | PIKFYVE | See above | 143-148 |
| | SYF2 | See above | 167-168 |
| | UNC13A | See above | 169, |
| | | | 810-818 |
| | C9orf72 | See above | 393-395 |
| | MAPT | See above | 506-534 |
| Motor neuron disease | APOE | See above | 501-505 |
| Epilepsy/pain | SCN1A | NR_148667.2; NM_001353949.2; | 546-563 |
| | | NM_001353958.2; NM_001353950.2; | |
| | | NM_001202435.3; NM_001353955.2; | |
| | | NM_001353957.2; NM_001353951.2; | |
| | | NM_001353948.2; NM_001353960.2; | |
| | | NM_001353954.2; XM_047445392.1; | |
| | | NM_001165964.3; NM_001353952.2; | |
| | | NM_001353961.2; NM_006920.6; | |
| | | NM_001165963.4; XM_047445393.1 | |
| | SCN2A | NM_001040143.2; NM_001371246.1; | 564-568 |
| | | NM_001040142.2; NM_001371247.1; | |
| | | NM_021007.3 | |
| | SCN8A | NM_014191.4; NM_001330260.2; | 569-572 |
| | | NM_001177984.3; NM_001369788.1 | |
| | SCN9A | XR_001738886.2; XM_011511618.3; | 573-580 |
| | | XM_011511617.3; NM_002977.3; | |
| | | XM_011511619.3; XM_011511616.4; | |
| | | NM_001365536.1; XM_017004669.2 | |
| | CLN3 | NM_001286109.2; NM_001286104.2; | 581-586 |
| | | NM_001286105.2; NM_001286110.2; | |
| | | NM_000086.2; NM_001042432.2 | |
| | GRIA1 | NR_157093.2; NR_047578.2; | 587-600 |
| | | XM_047417128.1; NM_001364166.2; | |
| | | NM_001364167.2; NM_001258020.2; | |
| | | NM_000827.4; NM_001114183.2; | |
| | | NM_001364165.2; NM_001258019.2; | |
| | | NM_001258023.1; NM_001258021.2; | |
| | | NM_001258022.2; XM_017009392.2 | |
| | PCDH19 | NM_020766.3; NM_001184880.2; | 601-604 |
| | | NM_001105243.2; XM_011530997.3 | |
| | GRIN2A | NM_000833.5; NM_001134407.3; | 838-840 |
| | | NM_001134408.2 | |
| Other | GYS1 | NM_002103.5; NM_001161587.2; | 605-607 |
| neurological | | NR_027763.2 | |
| diseases | PrP | NM_001080123.3; NM_000311.5; | 608-613 |
| | | NM_001080121.3; NM_001271561.3; | |
| | | NM_183079.4; NM_001080122.3 | |
| | GFAP | NM_002055.5; NM_001363846.2; | 614-617 |
| | | NM_001131019.3; NM_001242376.3 | |
| | LSD (e.g., | NM_001009999.3; XM_006710473.4; | 618-626 |
| | LSD1) | XM_006710474.4; XM_017000716.2; | |
| | | XM_005245786.3; XM_047449677.1; | |
| | | NM_001363654.2; XM_017000717.2; | |
| | | NM_015013.4 | |
| | SCN9A | See above | 573-580 |
| | UBE3A | NR_148916.2; XM_017022548.3; | 627-700 |
| | | XM_017022547.3; XM_047433008.1; | |
| | | NM_001354546.2; NM_001354551.2; | |
| | | NM_001354549.2; NM_001354548.2; | |
| | | NM_130838.4; NM_001354542.2; | |
| | | NM_001354550.2; NM_001354545.2; | |
| | | NM_130839.5; NM_001354523.2; | |
| | | NM_001354539.2; NM_001354511.2; | |
| | | NM_001354512.2; NM_001354513.2; | |
| | | NM_001354547.2; NM_001354508.2; | |
| | | NM_001354506.2; NM_001354544.2; | |
| | | NM_001354541.2; NM_001354509.2; | |
| | | NM_001354540.2; NM_001354543.2; | |
| | | NM_001354538.2; NM_001354507.2; | |
| | | XM_011521995.4; XM_047433015.1; | |
| | | XM_017022550.3; XM_047433009.1; | |
| | | XM_047433019.1; XM_047433021.1; | |
| | | XM_047433010.1; XM_047433014.1; | |
| | | XM_047433012.1; XM_047433013.1; | |
| | | XM_024450043.2; XM_047433018.1; | |
| | | XM_047433011.1; XM_047433017.1; | |
| | | XM_047433020.1; XM_047433007.1; | |
| | | XM_047433016.1; NM_001374461.1; | |
| | | NM_000462.5; NM_001354505.1; | |
| | | NM_001354526.1; NM_001354546.1; | |
| | | NM_001354551.1; NM_001354549.1; | |
| | | NM_001354548.1; NM_130838.3; | |
| | | NM_001354542.1; NM_001354550.1; | |
| | | NM_001354545.1; NM_130839.4; | |
| | | NM_001354523.1; NM_001354539.1; | |
| | | NM_001354511.1; NM_001354512.1; | |
| | | NM_001354513.1; NM_001354547.1; | |
| | | NM_001354508.1; NM_001354506.1; | |
| | | NM_001354544.1; NM_001354541.1; | |
| | | NM_001354509.1; NM_001354540.1; | |
| | | NM_001354543.1; NM_001354538.1; | |
| | | NM_001354507.1; NR_148916.1 | |
| | VLA-4 | NM_000885.6; NM_001316312.2 | 701-702 |
| GM1 | GLB1 | NM_000404.4; NM_001079811.3; | 841-845 |
| gangliosidosis | | NM_001135602.3; NM_001317040.2; | |
| | | NM_001393580.1 | |

In some embodiments, the molecular payload is an oligonucleotide that comprises a strand having a region of complementarity to a gene (e.g., a gene transcript) provided in Table 4.

TABLE 4
List of central nervous system diseases and corresponding genes.

| | Gene | | SEQ ID |
|---|---|---|---|
| Disease | Symbol | Transcripts GenBank Accession No. | NO: |
| Hereditary | TOR1A | NM_000113.3 | 705 |
| dystonia | THAP1 | NM_018105.3; NM_199003.2 | 706-707 |
| | ANO3 | NM_001313726.2; NM_031418.4; | 708-714 |
| | | XM_047427399.1; XM_017018118.3; | |
| | | NM_001313727.2; XM_017018119.3; | |
| | | XM_011520282.4 | |
| | GNAL | NM_182978.4; NM_001142339.3; | 715-720 |
| | | NM_001261443.2; NM_001261444.2; | |
| | | NM_001369387.1; XM_006722324.4 | |
| | KMT2B | NM_014727.3; XM_011527561.3; | 721-725 |
| | | XM_011527562.3; XM_047439787.1; | |
| | | XR_935878.3 | |
| | GCH1 | NM_001024071.2; NM_001024070.2; | 726-731 |
| | | NM_001024024.2; NM_000161.3; | |
| | | XM_017021218.2; XM_047431261.1 | |
| | TH | NM_000360.4; NM_199292.3; | 732-735 |
| | | NM_199293.3; XM_011520335.3 | |
| | SPR | NM_003124.5 | 736 |
| | TAF1 | NM_004606.5; NM_138923.4; | 737-767 |
| | | NM_001286074.2; NR_104387.2; | |
| | | NR_104388.2; NR_104389.2; | |
| | | NR_104390.2; NR_104391.2; | |
| | | NR_104392.2; NR_104393.2; | |
| | | NR_104394.2; NR_104395.2; | |
| | | NR_104396.2; XM_005262300.3; | |
| | | XM_024452430.2; XM_047442391.1; | |
| | | XM_047442392.1; XM_047442393.1; | |
| | | XM_047442394.1; XM_047442395.1; | |
| | | XM_047442396.1; XM_047442397.1; | |
| | | XM_047442398.1; XM_047442399.1; | |
| | | XM_047442400.1; XM_047442401.1; | |
| | | XM_047442402.1; XM_047442403.1; | |
| | | XM_047442404.1; XM_047442405.1; | |
| | | XM_047442406.1 | |
| | PRKRA | NM_003690.5; NM_001139517.1; | 768-773 |
| | | NM_001139518.1; NM_001316362.2; | |
| | | XM_011512063.3; XM_047446138.1 | |
| | ATP1A3 | NM_152296.5; NM_001256213.2; | 774-777 |
| | | NM_001256214.2; XM_047438862.1 | |
| | SGCE | NM_003919.3; NM_001099400.2; | 778-789 |
| | | NM_001099401.2; NM_001301139.2; | |
| | | NM_001346713.2; NM_001346715.2; | |
| | | NM_001346717.2; NM_001346719.2; | |
| | | NM_001346720.2; NM_001362807.2; | |
| | | NM_001362808.2; NM_001362809.2 | |
| | PNKD | NM_015488.5; NM_022572.4; | 790-794 |
| | | NM_001077399.3; XM_017003771.2; | |
| | | XM_017003772.2 | |
| | PRRT2 | NM_145239.3; NM_001256442.2; | 795-801 |
| | | NM_001256443.2; XM_011545715.4; | |
| | | XM_017022887.3; XM_017022888.3; | |
| | | XM_017022889.3 | |
| | SLC2A1 | NM_006516.4 | 802 |
| | ECHS1 | NM_004092.4 | 803 |
| Dravet syndrome | SCN1A | See above | 546-563 |
| Batten disease | TPP1 | NM_000391.4 | 846 |
| | CLN3 | See above | 581-586 |
| Niemann-Pick | ASM | NM_005343.4; NM_001007593.3; | 847-851 |
| Type A | | NM_001318087.2; NM_001318088.2; | |
| | | NM_001365135.2 | |
| Metachromatic | ARSA | NM_000487.6; NM_001085425.3; | 852-860 |
| leukodystrophy | | NM_001085426.3; NM_001085427.3; | |
| | | NM_001085428.3; NM_001362782.2; | |
| | | XM_047441363.1; XM_024452241.2; | |
| | | XM_011530691.4 | |
| Krabbe disease | GALC | NM_000153.4; NM_001201401.2; | 861-866 |
| | | NM_001201402.2; XM_011536618.3; | |
| | | XM_047431198.1; XM_047431199.1 | |
| Tay-Sachs | HEXA | NM_000520.6; NM_001318825.2 | 867-868 |
| Sandhoff disease | HEXB | NM_000521.4; NM_001292004.2 | 869-870 |
| Gaucher disease, | GBA | NM_000157.4; NM_001005741.3; | 871-875 |
| types II and III | | NM_001005742.3; NM_001171811.2; | |
| | | NM_001171812.2 | |
| Rett syndrome | MECP2 | NM_004992.4; NM_001110792.2; | 1059-1068 |
| | | NM_001316337.2; NM_001369391.2; | |
| | | NM_001369392.2; NM_001369393.2; | |
| | | NM_001369394.2; NM_001386137.1; | |
| | | NM_001386138.1; NM_001386139.1 | |

### Oligonucleotide Payloads

In some embodiments, oligonucleotides are useful in the treatment of various CNS diseases and disorders. For example, oligonucleotides may be useful to modulate the expression or activity of various genes involved in CNS diseases and disorder, such as by modulating transcription of the genes, modulating stability of mRNA molecules encoded by the genes, modulating translation of the mRNA molecules encoded by the genes, and modulating splicing of pre-mRNA transcripts encoded by the genes. Oligonucleotides may be used to treat various CNS diseases and disorders, for example, by facilitating delivery of the oligonucleotide into cells of the CNS. In some embodiments, oligonucleotides disclosed herein can be delivered into cells of the CNS using complexes disclosed here (e.g., anti-TfR1 antibody complexes comprising the oligonucleotide).

In some embodiments, oligonucleotides are useful in the treatment of a neuromuscular disease or disorder (e.g., Duchenne muscular dystrophy, myotonic dystrophy, Friedreich's ataxia, or spinal muscular atrophy); amyotrophic lateral sclerosis; Parkinson's disease; Huntington's disease; Alzheimer's disease; epilepsy; a pain disorder; glycogen synthesis disorders; neurodegeneration; small fiber neuropathy; nociception-related phenotypes; Alexander disease; Angelman Syndrome; autism-spectrum disorders; retinitis pigmentosa; isolated macular dystrophy; and/or multiple sclerosis.

In some embodiments, oligonucleotides are useful in the treatment of essential tremor and/or hereditary dystonia.

In some embodiments, oligonucleotides are useful in the modulation of one or more genes associated with a CNS disease or disorder. In some embodiments, the one or more genes associated with a CNS disease or disorder is DMPK, DMD, SMN, FXN, SOD1, C9orf72, ATXN2, FUS, LRRK2, SNCA, HTT, MSH3, TREM2, APOE, MAPT, APP, GYS1, PrP, VLA-4, GFAP, UBE3A, LSD, SCN9A, SCN1A, SCN2A, SCN8A, CLN3, GRIA1, or PCDH19. In some embodiments, the one or more genes associated with a CNS disease or disorder is TOR1A, THAP1, ANO3, GNAL, KMT2B, GCH1, TH, SPR, TAF1, PRKRA, ATP1A3, SGCE, PNKD, PRRT2, SLC2A1, or ECHS1. In some embodiments, the one or more genes associated with a CNS disease or disorder is PIKFYVE, SYF2, UNC13A, ATXN1, ATXN3, GRN, GRIN2A, TPP1, GLB1, ASM, ARSA, GALC, HEXA, HEXB, GBA, or MECP2.

In some embodiments, the 5′ or 3′ nucleoside (e.g., terminal nucleoside) of any one of the oligonucleotides described herein is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of any of the oligonucleotides described herein is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A}), —OC(═O)—, —OC(═O)O—, —OC(═O)N(R^{A})—, S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of any one of the oligonucleotides described herein is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)ₙ— and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a CNS targeting agent such as an anti-TfR1 antibody, e.g., via the amine group.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, 20 to 25 nucleotides in length, etc.

In some embodiments, a nucleic acid sequence of an oligonucleotide for purposes of the present disclosure is “complementary” to a target nucleic acid when it is specifically hybridizable to the target nucleic acid. In some embodiments, an oligonucleotide hybridizing to a target nucleic acid (e.g., a transcript provided in Table 3, e.g., provided by any one of SEQ ID NOs: 392-702) results in modulation of activity or expression of the target (e.g., decreased mRNA translation, altered pre-mRNA splicing, exon skipping, target mRNA degradation, etc.). In some embodiments, an oligonucleotide hybridizing to a target nucleic acid (e.g., a transcript provided in Table 4, e.g., provided by any one of SEQ ID NOs: 705-803) results in modulation of activity or expression of the target (e.g., decreased mRNA translation, altered pre-mRNA splicing, exon skipping, target mRNA degradation, etc.). In some embodiments, an oligonucleotide hybridizing to a target nucleic acid (e.g., a transcript provided in Table 3 or in Table 4, e.g., provided by any one of SEQ ID NOs: 143-148, 167-169, 810-875, and 1059-1068) results in modulation of activity or expression of the target (e.g., decreased mRNA translation, altered pre-mRNA splicing, exon skipping, target mRNA degradation, etc.). In some embodiments, an oligonucleotide hybridizing to a target nucleic acid results in an increase of activity or expression of the target (e.g., increased mRNA translation, such as of a wild-type form of the mRNA; altered pre-mRNA splicing; exon skipping; target mRNA stabilization; etc.). In some embodiments, a nucleic acid sequence of an oligonucleotide has a sufficient degree of complementarity to its target nucleic acid such that it does not hybridize non-target sequences under conditions in which avoidance of non-specific binding is desired, e.g., under physiological conditions. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of a target nucleic acid. In some embodiments a complementary nucleotide sequence need not be 100% complementary to that of its target to be specifically hybridizable or specific for a target nucleic acid. In certain embodiments, oligonucleotides comprise one or more mismatched nucleobases relative to the target nucleic acid. In certain embodiments, activity relating to the target is reduced by such mismatch, but activity relating to a non-target is reduced by a greater amount (i.e., selectivity for the target nucleic acid is increased and off-target effects are decreased).

In some embodiments, an oligonucleotide comprises region of complementarity to a target nucleic acid that is in the range of 8 to 15, 8 to 30, 8 to 40, or 10 to 50, or 5 to 50, 15 to 20, 20 to 25, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, the oligonucleotide is complementary (e.g., at least 85% at least 90%, at least 95%, or 100%) to a target sequence of the any one of the oligonucleotides described herein (e.g., the oligonucleotides listed in Tables 5-19). In some embodiments, the oligonucleotide is complementary (e.g., at least 85% at least 90%, at least 95%, or 100%) to a target sequence provided herein (e.g., a transcript listed in Table 3, e.g., provided by any one of SEQ ID NOs: 392-702). In some embodiments, the oligonucleotide is complementary (e.g., at least 85% at least 90%, at least 95%, or 100%) to a target sequence provided herein (e.g., a transcript listed in Table 4, e.g., provided by any one of SEQ ID NOs: 705-803). In some embodiments, the oligonucleotide is complementary (e.g., at least 85% at least 90%, at least 95%, or 100%) to a target sequence provided herein (e.g., a transcript listed in Table 3 or Table 4, e.g., provided by any one of SEQ ID NOs: 143-148, 167-169, 810-875, and 1059-1068).

In some embodiments, an oligonucleotide useful for targeting a transcript provided herein comprises a sequence comprising at least 8 (e.g., 8, 9, 10, 11, 12, 13, 14, 15, 16, 17, 18, 19, 20, 21, 22, 23, 24, 25, or more) consecutive nucleobases of an oligonucleotide sequence provided herein (e.g., an oligonucleotide sequence listed in any one of Tables 5-19). In some embodiments, an oligonucleotide useful for targeting a transcript provided herein comprises a sequence comprising a region of complementarity of at least 8 (e.g., 8, 9, 10, 11, 12, 13, 14, 15, 16, 17, 18, 19, 20, 21, 22, 23, 24, 25, or more) consecutive nucleobases complementary to a target sequence of an oligonucleotide sequence provided herein (e.g., an oligonucleotide sequence listed in any one of Tables 5-19).

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotides listed in any one of Tables 5-19) may independently and optionally be uracil bases (U's), and/or any one or more of the U's in the oligonucleotides provided herein may independently and optionally be T's.

b. Oligonucleotide Modifications:

The oligonucleotides described herein may be modified, e.g., comprise a modified sugar moiety, a modified internucleoside linkage, a modified nucleotide or nucleoside and/or (e.g., and) combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide or nucleoside modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide or nucleoside modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides or nucleosides of the oligonucleotide are modified nucleotides/nucleosides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides or nucleosides of the oligonucleotide are modified nucleotides/nucleosides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides or nucleosides of the oligonucleotide are modified nucleotides/nucleosides. Optionally, the oligonucleotides may have every nucleotide or nucleoside except 1, 2, 3, 4, 5, 6, 7, 8, 9, or 10 nucleotides/nucleosides modified. Oligonucleotide modifications are described further herein.

c. Modified Nucleosides

In some embodiments, the oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, the oligonucleotide described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleoside.

In some embodiments, the oligonucleotide described herein comprises one or more 2′-4′ bicyclic nucleosides in which the ribose ring comprises a bridge moiety connecting two atoms in the ring, e.g., connecting the 2′-0 atom to the 4′-C atom via a methylene (LNA) bridge, an ethylene (ENA) bridge, or a (S)-constrained ethyl (cEt) bridge. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. Nos. 7,101,993; 7,399,845 and 7,569,686, each of which is herein incorporated by reference in its entirety.

In some embodiments, the oligonucleotide comprises a modified nucleoside disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one modified nucleoside that results in an increase in Tm of the oligonucleotide in a range of 1° C., 2° C., 3° C., 4° C., or 5° C. compared with an oligonucleotide that does not have the at least one modified nucleoside. The oligonucleotide may have a plurality of modified nucleosides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleoside.

The oligonucleotide may comprise a mix of nucleosides of different kinds. For example, an oligonucleotide may comprise a mix of 2′-deoxyribonucleosides or ribonucleosides and 2′-fluoro modified nucleosides. An oligonucleotide may comprise a mix of deoxyribonucleosides or ribonucleosides and 2′-O-Me modified nucleosides. An oligonucleotide may comprise a mix of 2′-fluoro modified nucleosides and 2′-O-Me modified nucleosides. An oligonucleotide may comprise a mix of 2′-4′ bicyclic nucleosides and 2′-MOE, 2′-fluoro, or 2′-O-Me modified nucleosides. An oligonucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt).

The oligonucleotide may comprise alternating nucleosides of different kinds. For example, an oligonucleotide may comprise alternating 2′-deoxyribonucleosides or ribonucleosides and 2′-fluoro modified nucleosides. An oligonucleotide may comprise alternating deoxyribonucleosides or ribonucleosides and 2′-O-Me modified nucleosides. An oligonucleotide may comprise alternating 2′-fluoro modified nucleosides and 2′-O-Me modified nucleosides. An oligonucleotide may comprise alternating 2′-4′ bicyclic nucleosides and 2′-MOE, 2′-fluoro, or 2′-O-Me modified nucleosides. An oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In some embodiments, an oligonucleotide described herein comprises a 5′-vinylphosphonate modification, one or more abasic residues, and/or one or more inverted abasic residues.

d. Internucleoside Linkages/Backbones

In some embodiments, oligonucleotide may contain a phosphorothioate or other modified internucleoside linkage. In some embodiments, the oligonucleotide comprises phosphorothioate internucleoside linkages. In some embodiments, the oligonucleotide comprises phosphorothioate internucleoside linkages between at least two nucleosides. In some embodiments, the oligonucleotide comprises phosphorothioate internucleoside linkages between all nucleosides. For example, in some embodiments, oligonucleotides comprise modified internucleoside linkages at the first, second, and/or (e.g., and) third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides comprise nucleoside units that are joined together by either substantially all Sp or substantially all Rp phosphorothioate intersugar linkages are provided. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

f. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publications that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Mixmers

In some embodiments, an oligonucleotide described herein may be a mixmer or comprise a mixmer sequence pattern. In general, mixmers are oligonucleotides that comprise both naturally and non-naturally occurring nucleosides or comprise two different types of non-naturally occurring nucleosides typically in an alternating pattern. Mixmers generally have higher binding affinity than unmodified oligonucleotides and may be used to specifically bind a target molecule, e.g., to block a binding site on the target molecule. Generally, mixmers do not recruit an RNase to the target molecule and thus do not promote cleavage of the target molecule. Such oligonucleotides that are incapable of recruiting RNase H have been described, for example, see WO2007/112754 or WO2007/112753.

In some embodiments, the mixmer comprises or consists of a repeating pattern of nucleoside analogues and naturally occurring nucleosides, or one type of nucleoside analogue and a second type of nucleoside analogue. However, a mixmer need not comprise a repeating pattern and may instead comprise any arrangement of modified nucleoside s and naturally occurring nucleoside s or any arrangement of one type of modified nucleoside and a second type of modified nucleoside. The repeating pattern, may, for instance be every second or every third nucleoside is a modified nucleoside, such as LNA, and the remaining nucleoside s are naturally occurring nucleosides, such as DNA, or are a 2′ substituted nucleoside analogue such as 2′-MOE or 2′ fluoro analogues, or any other modified nucleoside described herein. It is recognized that the repeating pattern of modified nucleoside, such as LNA units, may be combined with modified nucleoside at fixed positions—e.g. at the 5′ or 3′ termini.

In some embodiments, a mixmer does not comprise a region of more than 5, more than 4, more than 3, or more than 2 consecutive naturally occurring nucleosides, such as DNA nucleosides. In some embodiments, the mixmer comprises at least a region consisting of at least two consecutive modified nucleosides, such as at least two consecutive LNAs. In some embodiments, the mixmer comprises at least a region consisting of at least three consecutive modified nucleoside units, such as at least three consecutive LNAs.

In some embodiments, the mixmer does not comprise a region of more than 7, more than 6, more than 5, more than 4, more than 3, or more than 2 consecutive nucleoside analogues, such as LNAs. In some embodiments, LNA units may be replaced with other nucleoside analogues, such as those referred to herein.

Mixmers may be designed to comprise a mixture of affinity enhancing modified nucleosides, such as in non-limiting example LNA nucleosides and 2′-O-Me nucleosides. In some embodiments, a mixmer comprises modified internucleoside linkages (e.g., phosphorothioate internucleoside linkages or other linkages) between at least two, at least three, at least four, at least five or more nucleosides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, a mixmer comprises one or more morpholino nucleosides. For example, in some embodiments, a mixmer may comprise morpholino nucleosides mixed (e.g., in an alternating manner) with one or more other nucleosides (e.g., DNA, RNA nucleosides) or modified nucleosides (e.g., LNA, 2′-O-Me nucleosides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)-Uridine Phosphoramidite, and Exon Skipping Using MNA/2′-O-Methyl Mixmer Antisense Oligonucleotide, Molecules 2016, 21, 1582, the contents of each which are incorporated herein by reference.

i. Multimers

In some embodiments, molecular payloads may comprise multimers (e.g., concatemers) of 2 or more oligonucleotides connected by a linker. In this way, in some embodiments, the oligonucleotide loading of a complex can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via an oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

j. Gapmers

In some embodiments, the oligonucleotide described herein is a gapmer. A gapmer oligonucleotide generally has the formula 5′-X-Y-Z-3′, with X and Z as flanking regions around a gap region Y. In some embodiments, flanking region X of formula 5′-X-Y-Z-3′ is also referred to as X region, flanking sequence X, 5′ wing region X, or 5′ wing segment. In some embodiments, flanking region Z of formula 5′-X-Y-Z-3′ is also referred to as Z region, flanking sequence Z, 3′ wing region Z, or 3′ wing segment. In some embodiments, gap region Y of formula 5′-X-Y-Z-3′ is also referred to as Y region, Y segment, or gap-segment Y. In some embodiments, each nucleoside in the gap region Y is a 2′-deoxyribonucleoside, and neither the 5′ wing region X or the 3′ wing region Z contains any 2′-deoxyribonucleosides. In some embodiments, a gapmer oligonucleotide comprises a region of complementarity to at least 15 consecutive nucleosides (e.g., at least 15, at least 16, at least 17, at least 18, at least 19, or 20 consecutive nucleosides) of a target nucleic acid sequence provided herein (e.g., a transcript listed in Table 3, e.g., provided by any one of SEQ ID NOs: 392-702, or a target sequence of any of the oligonucleotides listed in Tables 5-19) and/or comprises at least 15 consecutive nucleosides (e.g., at least 15, at least 16, at least 17, at least 18, at least 19, or 20 consecutive nucleosides) of the nucleotide sequence of an oligonucleotide sequence in any one of Tables 5-19, wherein each thymine base (T) may independently and optionally be replaced with a uracil base (U), and each U may independently and optionally be replaced with a T. In some embodiments, a gapmer oligonucleotide comprises a region of complementarity to at least 15 consecutive nucleosides (e.g., at least 15, at least 16, at least 17, at least 18, at least 19, or 20 consecutive nucleosides) of a target nucleic acid sequence provided herein (e.g., a transcript listed in Table 4, e.g., provided by any one of SEQ ID NOs: 705-803). In some embodiments, a gapmer oligonucleotide comprises a region of complementarity to at least 15 consecutive nucleosides (e.g., at least 15, at least 16, at least 17, at least 18, at least 19, or 20 consecutive nucleosides) of a target nucleic acid sequence provided herein (e.g., a transcript listed in Table 3 or Table 4, e.g., provided by any one of SEQ ID NOs: 143-148, 167-169, 810-875, and 1059-1068).

In some embodiments, the Y region is a contiguous stretch of nucleotides, e.g., a region of 6 or more DNA nucleotides, which are capable of recruiting an RNAse, such as RNAse H. In some embodiments, the gapmer binds to the target nucleic acid, at which point an RNAse is recruited and can then cleave the target nucleic acid. In some embodiments, the Y region is flanked both 5′ and 3′ by regions X and Z comprising high-affinity modified nucleosides, e.g., one to six high-affinity modified nucleosides. Examples of high affinity modified nucleosides include, but are not limited to, 2′-modified nucleosides (e.g., 2′-MOE, 2′O-Me, 2′-F) or 2′-4′ bicyclic nucleosides (e.g., LNA, cEt, ENA). In some embodiments, the flanking sequences X and Z may be of 1-20 nucleotides, 1-8 nucleotides, or 1-5 nucleotides in length. The flanking sequences X and Z may be of similar length or of dissimilar lengths. In some embodiments, the gap-segment Y may be a nucleotide sequence of 5-20 nucleotides, 5-15 nucleotides, 5-12 nucleotides, or 6-10 nucleotides in length.

In some embodiments, the gap region of the gapmer oligonucleotides may contain modified nucleosides known to be acceptable for efficient RNase H action in addition to DNA nucleosides, such as C4′-substituted nucleosides, acyclic nucleosides, and arabino-configured nucleosides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using appropriate methods. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,015,315; 7,101,993; 7,399,845; 7,432,250; 7,569,686; 7,683,036; 7,750,131; 8,580,756; 9,045,754; 9,428,534; 9,695,418; 10,017,764; 10,260,069; 9,428,534; 8,580,756; U.S. patent publication Nos. US20050074801, US20090221685; US20090286969, US20100197762, and US20110112170; PCT publication Nos. WO2004069991; WO2005023825; WO2008049085 and WO2009090182; and EP Patent No. EP2,149,605, each of which is herein incorporated by reference in its entirety.

In some embodiments, the gapmer is 10-40 nucleosides in length. For example, the gapmer may be 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 nucleosides in length. In some embodiments, the gapmer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleosides in length.

In some embodiments, the gap region Y in the gapmer is 5-20 nucleosides in length. For example, the gap region Y may be 5-20, 5-15, 5-10, 10-20, 10-15, or 15-20 nucleosides in length. In some embodiments, the gap region Y is 5, 6, 7, 8, 9, 10, 11, 12, 13, 14, 15, 16, 17, 18, 19, or 20 nucleosides in length. In some embodiments, each nucleoside in the gap region Y is a 2′-deoxyribonucleoside. In some embodiments, all nucleosides in the gap region Y are 2′-deoxyribonucleosides. In some embodiments, one or more of the nucleosides in the gap region Y is a modified nucleoside (e.g., a 2′ modified nucleoside such as those described herein). In some embodiments, one or more cytosines in the gap region Y are optionally 5-methyl-cytosines. In some embodiments, each cytosine in the gap region Y is a 5-methyl-cytosine.

In some embodiments, the 5′ wing region of the gapmer (X in the 5′-X-Y-Z-3′ formula) and the 3′ wing region of the gapmer (Z in the 5′-X-Y-Z-3′ formula) are independently 1-20 nucleosides long. For example, the 5′ wing region of the gapmer (X in the 5′-X-Y-Z-3′ formula) and the 3′ wing region of the gapmer (Z in the 5′-X-Y-Z-3′ formula) may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the 5′ wing region of the gapmer (X in the 5′-X-Y-Z-3′ formula) and the 3′ wing region of the gapmer (Z in the 5′-X-Y-Z-3′ formula) are independently 1, 2, 3, 4, 5, 6, 7, 8, 9, 10, 11, 12, 13, 14, 15, 16, 17, 18, 19, or 20 nucleosides long. In some embodiments, the 5′ wing region of the gapmer (X in the 5′-X-Y-Z-3′ formula) and the 3′ wing region of the gapmer (Z in the 5′-X-Y-Z-3′ formula) are of the same length. In some embodiments, the 5′ wing region of the gapmer (X in the 5′-X-Y-Z-3′ formula) and the 3′ wing region of the gapmer (Z in the 5′-X-Y-Z-3′ formula) are of different lengths. In some embodiments, the 5′ wing region of the gapmer (X in the 5′-X-Y-Z-3′ formula) is longer than the 3′ wing region of the gapmer (Z in the 5′-X-Y-Z-3′ formula). In some embodiments, the 5′ wing region of the gapmer (X in the 5′-X-Y-Z-3′ formula) is shorter than the 3′ wing region of the gapmer (Z in the 5′-X-Y-Z-3′ formula).

In some embodiments, the gapmer comprises a 5′-X-Y-Z-3′ of 5-10-5, 4-12-4, 3-14-3, 2-16-2, 1-18-1, 3-10-3, 2-10-2, 1-10-1, 2-8-2, 4-6-4, 3-6-3, 2-6-2, 4-7-4, 3-7-3, 2-7-2, 4-8-4, 3-8-3, 2-8-2, 1-8-1, 2-9-2, 1-9-1, 2-10-2, 1-10-1, 1-12-1, 1-16-1, 2-15-1, 1-15-2, 1-14-3, 3-14-1, 2-14-2, 1-13-4, 4-13-1, 2-13-3, 3-13-2, 1-12-5, 5-12-1, 2-12-4, 4-12-2, 3-12-3, 1-11-6, 6-11-1, 2-11-5, 5-11-2, 3-11-4, 4-11-3, 1-17-1, 2-16-1, 1-16-2, 1-15-3, 3-15-1, 2-15-2, 1-14-4, 4-14-1, 2-14-3, 3-14-2, 1-13-5, 5-13-1, 2-13-4, 4-13-2, 3-13-3, 1-12-6, 6-12-1, 2-12-5, 5-12-2, 3-12-4, 4-12-3, 1-11-7, 7-11-1, 2-11-6, 6-11-2, 3-11-5, 5-11-3, 4-11-4, 1-18-1, 1-17-2, 2-17-1, 1-16-3, 1-16-3, 2-16-2, 1-15-4, 4-15-1, 2-15-3, 3-15-2, 1-14-5, 5-14-1, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-18-1, 1-17-2, 2-17-1, 1-16-3, 3-16-1, 2-16-2, 1-15-4, 4-15-1, 2-15-3, 3-15-2, 1-14-5, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-19-1, 1-18-2, 2-18-1, 1-17-3, 3-17-1, 2-17-2, 1-16-4, 4-16-1, 2-16-3, 3-16-2, 1-15-5, 2-15-4, 4-15-2, 3-15-3, 1-14-6, 6-14-1, 2-14-5, 5-14-2, 3-14-4, 4-14-3, 1-13-7, 7-13-1, 2-13-6, 6-13-2, 3-13-5, 5-13-3, 4-13-4, 1-12-8, 8-12-1, 2-12-7, 7-12-2, 3-12-6, 6-12-3, 4-12-5, 5-12-4, 2-11-8, 8-11-2, 3-11-7, 7-11-3, 4-11-6, 6-11-4, 5-11-5, 1-20-1, 1-19-2, 2-19-1, 1-18-3, 3-18-1, 2-18-2, 1-17-4, 4-17-1, 2-17-3, 3-17-2, 1-16-5, 2-16-4, 4-16-2, 3-16-3, 1-15-6, 6-15-1, 2-15-5, 5-15-2, 3-15-4, 4-15-3, 1-14-7, 7-14-1, 2-14-6, 6-14-2, 3-14-5, 5-14-3, 4-14-4, 1-13-8, 8-13-1, 2-13-7, 7-13-2, 3-13-6, 6-13-3, 4-13-5, 5-13-4, 2-12-8, 8-12-2, 3-12-7, 7-12-3, 4-12-6, 6-12-4, 5-12-5, 3-11-8, 8-11-3, 4-11-7, 7-11-4, 5-11-6, 6-11-5, 1-21-1, 1-20-2, 2-20-1, 1-20-3, 3-19-1, 2-19-2, 1-18-4, 4-18-1, 2-18-3, 3-18-2, 1-17-5, 2-17-4, 4-17-2, 3-17-3, 1-16-6, 6-16-1, 2-16-5, 5-16-2, 3-16-4, 4-16-3, 1-15-7, 7-15-1, 2-15-6, 6-15-2, 3-15-5, 5-15-3, 4-15-4, 1-14-8, 8-14-1, 2-14-7, 7-14-2, 3-14-6, 6-14-3, 4-14-5, 5-14-4, 2-13-8, 8-13-2, 3-13-7, 7-13-3, 4-13-6, 6-13-4, 5-13-5, 1-12-10, 10-12-1, 2-12-9, 9-12-2, 3-12-8, 8-12-3, 4-12-7, 7-12-4, 5-12-6, 6-12-5, 4-11-8, 8-11-4, 5-11-7, 7-11-5, 6-11-6, 1-22-1, 1-21-2, 2-21-1, 1- 21-3, 3-20-1, 2-20-2, 1-19-4, 4-19-1, 2-19-3, 3-19-2, 1-18-5, 2-18-4, 4-18-2, 3-18-3, 1-17-6, 6- 17-1, 2-17-5, 5-17-2, 3-17-4, 4-17-3, 1-16-7, 7-16-1, 2-16-6, 6-16-2, 3-16-5, 5-16-3, 4-16-4, 1- 15-8, 8-15-1, 2-15-7, 7-15-2, 3-15-6, 6-15-3, 4-15-5, 5-15-4, 2-14-8, 8-14-2, 3-14-7, 7-14-3, 4- 14-6, 6-14-4, 5-14-5, 3-13-8, 8-13-3, 4-13-7, 7-13-4, 5-13-6, 6-13-5, 4-12-8, 8-12-4, 5-12-7, 7- 12-5, 6-12-6, 5-11-8, 8-11-5, 6-11-7, or 7-11-6. The numbers indicate the number of nucleosides in X, Y, and Z regions in the 5′-X-Y-Z-3′ gapmer.

In some embodiments, one or more nucleosides in the 5′ wing region of the gapmer (X in the 5′-X-Y-Z-3′ formula) or the 3′ wing region of the gapmer (Z in the 5′-X-Y-Z-3′ formula) are modified nucleosides (e.g., high-affinity modified nucleosides). In some embodiments, the modified nucleoside (e.g., high-affinity modified nucleosides) is a 2′-modified nucleoside. In some embodiments, the 2′-modified nucleoside is a 2′-4′ bicyclic nucleoside or a non-bicyclic 2′-modified nucleoside. In some embodiments, the high-affinity modified nucleoside is a 2′-4′ bicyclic nucleoside (e.g., LNA, cEt, or ENA) or a non-bicyclic 2′-modified nucleoside (e.g., 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA)).

In some embodiments, one or more nucleosides in the 5′ wing region of the gapmer (X in the 5′-X-Y-Z-3′ formula) are high-affinity modified nucleosides. In some embodiments, each nucleoside in the 5′ wing region of the gapmer (X in the 5′-X-Y-Z-3′ formula) is a high-affinity modified nucleoside. In some embodiments, one or more nucleosides in the 3′ wing region of the gapmer (Z in the 5′-X-Y-Z-3′ formula) are high-affinity modified nucleosides. In some embodiments, each nucleoside in the 3′ wing region of the gapmer (Z in the 5′-X-Y-Z-3′ formula) is a high-affinity modified nucleoside. In some embodiments, one or more nucleosides in the 5′ wing region of the gapmer (X in the 5′-X-Y-Z-3′ formula) are high-affinity modified nucleosides and one or more nucleosides in the 3′ wing region of the gapmer (Z in the 5′-X-Y-Z-3′ formula) are high-affinity modified nucleosides. In some embodiments, each nucleoside in the 5′ wing region of the gapmer (X in the 5′-X-Y-Z-3′ formula) is a high-affinity modified nucleoside and each nucleoside in the 3′wing region of the gapmer (Z in the 5′-X-Y-Z-3′ formula) is high-affinity modified nucleoside.

In some embodiments, the 5′ wing region of the gapmer (X in the 5′-X-Y-Z-3′ formula) comprises the same high affinity nucleosides as the 3′ wing region of the gapmer (Z in the 5′-X-Y-Z-3′ formula). For example, the 5′ wing region of the gapmer (X in the 5′-X-Y-Z-3′ formula) and the 3′ wing region of the gapmer (Z in the 5′-X-Y-Z-3′ formula) may comprise one or more non-bicyclic 2′-modified nucleosides (e.g., 2′-MOE or 2′-O-Me). In another example, the 5′ wing region of the gapmer (X in the 5′-X-Y-Z-3′ formula) and the 3′ wing region of the gapmer (Z in the 5′-X-Y-Z-3′ formula) may comprise one or more 2′-4′ bicyclic nucleosides (e.g., LNA or cEt). In some embodiments, each nucleoside in the 5′ wing region of the gapmer (X in the 5′-X-Y-Z-3′ formula) and the 3′ wing region of the gapmer (Z in the 5′-X-Y-Z-3′ formula) is a non-bicyclic 2′-modified nucleoside (e.g., 2′-MOE or 2′-O-Me). In some embodiments, each nucleoside in the 5′ wing region of the gapmer (X in the 5′-X-Y-Z-3′ formula) and the 3′ wing region of the gapmer (Z in the 5′-X-Y-Z-3′ formula) is a 2′-4′ bicyclic nucleoside (e.g., LNA or cEt).

In some embodiments, the gapmer comprises a 5′-X-Y-Z-3′ configuration, wherein X and Z are independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a non-bicyclic 2′-modified nucleosides (e.g., 2′-MOE or 2′-O-Me) and each nucleoside in Y is a 2′-deoxyribonucleoside. In some embodiments, the gapmer comprises a 5′-X-Y-Z-3′ configuration, wherein X and Z are independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a 2′-4′ bicyclic nucleosides (e.g., LNA or cEt) and each nucleoside in Y is a 2′-deoxyribonucleoside. In some embodiments, the 5′ wing region of the gapmer (X in the 5′-X-Y-Z-3′ formula) comprises different high affinity nucleosides as the 3′ wing region of the gapmer (Z in the 5′-X-Y-Z-3′ formula). For example, the 5′ wing region of the gapmer (X in the 5′-X-Y-Z-3′ formula) may comprise one or more non-bicyclic 2′-modified nucleosides (e.g., 2′-MOE or 2′-O-Me) and the 3′ wing region of the gapmer (Z in the 5′-X-Y-Z-3′ formula) may comprise one or more 2′-4′ bicyclic nucleosides (e.g., LNA or cEt). In another example, the 3′ wing region of the gapmer (Z in the 5′-X-Y-Z-3′ formula) may comprise one or more non-bicyclic 2′-modified nucleosides (e.g., 2′-MOE or 2′-O-Me) and the 5′ wing region of the gapmer (X in the 5′-X-Y-Z-3′ formula) may comprise one or more 2′-4′ bicyclic nucleosides (e.g., LNA or cEt).

In some embodiments, the gapmer comprises a 5′-X-Y-Z-3′ configuration, wherein X and Z are independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a non-bicyclic 2′-modified nucleoside (e.g., 2′-MOE or 2′-O-Me), each nucleoside in Z is a 2′-4′ bicyclic nucleoside (e.g., LNA or cEt), and each nucleoside in Y is a 2′-deoxyribonucleoside. In some embodiments, the gapmer comprises a 5′-X-Y-Z-3′ configuration, wherein X and Z are independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a 2′-4′ bicyclic nucleoside (e.g., LNA or cEt), each nucleoside in Z is a non-bicyclic 2′-modified nucleoside (e.g., 2′-MOE or 2′-O-Me) and each nucleoside in Y is a 2′-deoxyribonucleoside.

In some embodiments, the 5′ wing region of the gapmer (X in the 5′-X-Y-Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, the 3′ wing region of the gapmer (Z in the 5′-X-Y-Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, both the 5′ wing region of the gapmer (X in the 5′-X-Y-Z-3′ formula) and the 3′ wing region of the gapmer (Z in the 5′-X-Y-Z-3′ formula) comprise one or more non-bicyclic 2′-modified nucleosides (e.g., 2′-MOE or 2′-O-Me) and one or more 2′-4′ bicyclic nucleosides (e.g., LNA or cEt).

In some embodiments, the gapmer comprises a 5′-X-Y-Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X-Y-Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in Z (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X-Y-Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X and at least one of positions but not all (e.g., 1, 2, 3, 4, 5, or 6) of positions 1, 2, 3, 4, 5, 6, or 7 in Z (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside.

Non-limiting examples of gapmers configurations with a mix of non-bicyclic 2′-modified nucleoside (e.g., 2′-MOE or 2′-O-Me) and 2′-4′ bicyclic nucleosides (e.g., LNA or cEt) in the 5′wing region of the gapmer (X in the 5′-X-Y-Z-3′ formula) and/or the 3′wing region of the gapmer (Z in the 5′-X-Y-Z-3′ formula) include: BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BABA-(D)n-ABAB; KAKA-(D)n-AKAK; LALA-(D)n-ALAL; BEBE-(D)n-EBEB; KEKE-(D)n-EKEK; LELE-(Dn-ELEL; BABA-(Dn-ABAB; KAKA-(Dn-AKAK; LALA-(Dn-ALAL; BEBE-(Dn-EBEB; KEKE-(Dn-EKEK; LELE-(Dn-ELEL; ABAB-(Dn-ABAB; AKAK-(Dn-AKAK; ALAL-(Dn-ALAL; EBEB-(Dn-EBEB; EKEK-(Dn-EKEK; ELEL-(Dn-ELEL; ABAB-(Dn-ABAB; AKAK-(Dn-AKAK; ALAL-(Dn-ALAL; EBEB-(Dn-EBEB; EKEK-(Dn-EKEK; ELEL-(Dn-ELEL; AABB-(Dn-BBAA; BBAA-(Dn-AABB; AAKK-(Dn-KKAA; AALL-(Dn-LLAA; EEBB-(Dn-BBEE; EEKK-(Dn-KKEE; EELL-(Dn-LLEE; AABB-(Dn-BBAA; AAKK-(Dn-KKAA; AALL-(Dn-LLAA; EEBB-(Dn-BBEE; EEKK-(Dn-KKEE; EELL-(Dn-LLEE; BBB-(Dn-BBA; KKK-(Dn-KKA; LLL-(Dn-LLA; BBB-(Dn-BBE; KKK-(Dn-KKE; LLL-(Dn-LLE; BBB-(Dn-BBA; KKK-(Dn-KKA; LLL-(Dn-LLA; BBB-(Dn-BBE; KKK-(Dn-KKE; LLL-(Dn-LLE; BBB-(Dn-BBA; KKK-(Dn-KKA; LLL-(Dn-LLA; BBB-(Dn-BBE; KKK-(Dn-KKE; LLL-(Dn-LLE; ABBB-(Dn-BBBA; AKKK-(Dn-KKKA; ALLL-(Dn-LLLA; EBBB-(Dn-BBBE; EKKK-(Dn-KKKE; ELLL-(Dn-LLLE; ABBB-(Dn-BBBA; AKKK-(Dn-KKKA; ALLL-(Dn-LLLA; EBBB-(Dn-BBBE; EKKK-(Dn-KKKE; ELLL-(Dn-LLLE; ABBB-(Dn-BBBAA; AKKK-(Dn-KKKAA; ALLL-(Dn-LLLAA; EBBB-(Dn-BBBEE; EKKK-(Dn-KKKEE; ELLL-(Dn-LLLEE; ABBB-(Dn-BBBAA; AKKK-(Dn-KKKAA; ALLL-(Dn-LLLAA; EBBB-(Dn-BBBEE; EKKK-(Dn-KKKEE; ELLL-(Dn-LLLEE; AABBB-(Dn-BBB; AAKKK-(Dn-KKK; AALLL-(Dn-LLL; EEBBB-(Dn-BBB; EEKKK-(Dn-KKK; EELLL-(Dn-LLL; AABBB-(Dn-BBB; AAKKK-(Dn-KKK; AALLL-(Dn-LLL; EEBBB-(Dn-BBB; EEKKK-(Dn-KKK; EELLL-(Dn-LLL; AABBB-(Dn-BBBA; AAKKK-(Dn-KKKA; AALLL-(Dn-LLLA; EEBBB-(Dn-BBBE; EEKKK-(Dn-KKKE; EELLL-(Dn-LLLE; AABBB-(Dn-BBBA; AAKKK-(Dn-KKKA; AALLL-(Dn-LLLA; EEBBB-(Dn-BBBE; EEKKK-(Dn-KKKE; EELLL-(Dn-LLLE; ABBAABB-(Dn-BB; AKKAAKK-(Dn-KK; ALLAALLL-(Dn-LL; EBBEEBB-(Dn-BB; EKKEEKK-(Dn-KK; ELLEELL-(Dn-LL; ABBAABB-(Dn-BB; AKKAAKK-(Dn-KK; ALLAALL-(Dn-LL; EBBEEBB-(Dn-BB; EKKEEKK-(Dn-KK; ELLEELL-(Dn-LL; ABBABB-(Dn-BBB; AKKAKK-(Dn-KKK; ALLALLL-(Dn-LLL; EBBEBB-(Dn-BBB; EKKEKK-(Dn-KKK; ELLELL-(Dn-LLL; ABBABB-(Dn-BBB; AKKAKK-(Dn-KKK; ALLALL-(Dn-LLL; EBBEBB-(Dn-BBB; EKKEKK-(Dn-KKK; ELLELL-(Dn-LLL; EEEK-(Dn-EEEEEEEE; EEK-(D)n-EEEEEEEEE; EK-(D)n-EEEEEEEEEE; EK-(D)n-EEEKK; K-(D)n-EEEKEKE; K-(D)n-EEEKEKEE; K-(D)n-EEKEK; EK-(D)n-EEEEKEKE; EK-(D)n-EEEKEK; EEK-(D)n-KEEKE; EK-(D)n-EEKEK; EK-(D)n-KEEK; EEK-(D)n-EEEKEK; EK-(D)n-KEEEKEE; EK-(D)n-EEKEKE; EK-(D)n-EEEKEKE; and EK-(D)n-EEEEKEK; wherein “A” represents a 2′-modified nucleoside; “B” represents a 2′-4′ bicyclic nucleoside; “K” represents a constrained ethyl nucleoside (cEt); “L” represents an LNA nucleoside; and “E” represents a 2′-MOE modified ribonucleoside; “D” represents a 2′-deoxyribonucleoside; “n” represents the length of the gap segment (Y in the 5′-X-Y-Z-3′ configuration) and is an integer between 1-20.

In some embodiments, any one of the gapmers described herein comprises one or more modified nucleoside linkages (e.g., a phosphorothioate linkage) in each of the X, Y, and Z regions. In some embodiments, each internucleoside linkage in the any one of the gapmers described herein is a phosphorothioate linkage. In some embodiments, each of the X, Y, and Z regions independently comprises a mix of phosphorothioate linkages and phosphodiester linkages. In some embodiments, each internucleoside linkage in the gap region Y is a phosphorothioate linkage, the 5′ wing region X comprises a mix of phosphorothioate linkages and phosphodiester linkages, and the 3′ wing region Z comprises a mix of phosphorothioate linkages and phosphodiester linkages.

### Polypeptide Payloads

In some embodiments, polypeptides (e.g., peptides, proteins, including but not limited to enzymes, antibodies, etc.) are useful in the treatment of various CNS diseases and disorders. For example, polypeptides may be useful to modulate the expression or activity of various genes involved in CNS diseases and disorder, such as by modulating expression or activity of a protein involved in the CNS disease or disorder. In one non-limiting example, an enzyme that modifies, degrades, or otherwise affects a particular biological molecule (e.g., a protein or nucleic acid) may be useful in the treatment of a CNS disease or disorder involving that particular biological molecule. Polypeptides may be used to treat various CNS diseases and disorders, for example, by facilitating delivery of the polypeptide into cells of the CNS. In some embodiments, polypeptides disclosed herein can be delivered into cells of the CNS using complexes disclosed here (e.g., anti-TfR1 antibody complexes comprising the polypeptide).

In some embodiments, polypeptides are useful in the treatment of a neuromuscular disease or disorder (e.g., Duchenne muscular dystrophy, myotonic dystrophy, Friedreich's ataxia, or spinal muscular atrophy); amyotrophic lateral sclerosis; Parkinson's disease; Huntington's disease; Alzheimer's disease; epilepsy; a pain disorder; glycogen synthesis disorders; neurodegeneration; small fiber neuropathy; nociception-related phenotypes; Alexander disease; Angelman Syndrome; autism-spectrum disorders; retinitis pigmentosa; isolated macular dystrophy; and/or multiple sclerosis.

In some embodiments, polypeptides are useful in the treatment of essential tremor and/or hereditary dystonia.

In some embodiments, polypeptides are useful in the treatment of spinocerebellar ataxia, motor neuron disease, Dravet syndrome, Batten disease, GM1 gangliosidosis, Niemann-Pick Type A, metachromatic leukodystrophy, Krabbe disease, Tay-Sachs, Sandhoff disease, Gaucher disease types II and III, and/or Rett syndrome.

In some embodiments, polypeptides are useful in the modulation of one or more genes associated with a CNS disease or disorder. In some embodiments, the one or more genes associated with a CNS disease or disorder is DMPK, DMD, SMN, FXN, SOD1, C9orf72, ATXN2, FUS, LRRK2, SNCA, HTT, MSH3, TREM2, APOE, MAPT, APP, GYS1, PrP, VLA-4, GFAP, UBE3A, LSD, SCN9A, SCN1A, SCN2A, SCN8A, CLN3, GRIA1, or PCDH19. In some embodiments, the one or more genes associated with a CNS disease or disorder is TOR1A, THAP1, ANO3, GNAL, KMT2B, GCH1, TH, SPR, TAF1, PRKRA, ATP1A3, SGCE, PNKD, PRRT2, SLC2A1, or ECHS1. In some embodiments, the one or more genes associated with a CNS disease or disorder is PIKFYVE, SYF2, UNC13A, ATXN1, ATXN3, GRN, GRIN2A, TPP1, GLB1, ASM, ARSA, GALC, HEXA, HEXB, GBA, or MECP2.

### Small Molecule Payloads

In some embodiments, small molecules are useful in the treatment of various CNS diseases and disorders. For example, small molecules may be useful to modulate the expression or activity of various genes involved in CNS diseases and disorder, such as by modulating expression or activity of a protein involved in the CNS disease or disorder. In one non-limiting example, small molecule that increases, decreases, or otherwise affects expression of a particular biological molecule (e.g., a protein or nucleic acid) may be useful in the treatment of a CNS disease or disorder involving that particular biological molecule. Small molecules may be used to treat various CNS diseases and disorders, for example, by facilitating delivery of the small molecule into cells of the CNS. In some embodiments, small molecules disclosed herein can be delivered into cells of the CNS using complexes disclosed here (e.g., anti-TfR1 antibody complexes comprising the small molecule).

In some embodiments, small molecules are useful in the treatment of a neuromuscular disease or disorder (e.g., Duchenne muscular dystrophy, myotonic dystrophy, Friedreich's ataxia, or spinal muscular atrophy); amyotrophic lateral sclerosis; Parkinson's disease; Huntington's disease; Alzheimer's disease; epilepsy; a pain disorder; glycogen synthesis disorders; neurodegeneration; small fiber neuropathy; nociception-related phenotypes; Alexander disease; Angelman Syndrome; autism-spectrum disorders; retinitis pigmentosa; isolated macular dystrophy; and/or multiple sclerosis.

In some embodiments, small molecules are useful in the treatment of essential tremor and/or hereditary dystonia.

In some embodiments, small molecules are useful in the treatment of spinocerebellar ataxia, motor neuron disease, Dravet syndrome, Batten disease, GM1 gangliosidosis, Niemann-Pick Type A, metachromatic leukodystrophy, Krabbe disease, Tay-Sachs, Sandhoff disease, Gaucher disease types II and III, and/or Rett syndrome.

In some embodiments, small molecules are useful in the modulation of one or more genes associated with a CNS disease or disorder. In some embodiments, the one or more genes associated with a CNS disease or disorder is DMPK, DMD, SMN, FXN, SOD1, C9orf72, ATXN2, FUS, LRRK2, SNCA, HTT, MSH3, TREM2, APOE, MAPT, APP, GYS1, PrP, VLA-4, GFAP, UBE3A, LSD, SCN9A, SCN1A, SCN2A, SCN8A, CLN3, GRIA1, or PCDH19. In some embodiments, the one or more genes associated with a CNS disease or disorder is TOR1A, THAP1, ANO3, GNAL, KMT2B, GCH1, TH, SPR, TAF1, PRKRA, ATP1A3, SGCE, PNKD, PRRT2, SLC2A1, or ECHS1. In some embodiments, the one or more genes associated with a CNS disease or disorder is PIKFYVE, SYF2, UNC13A, ATXN1, ATXN3, GRN, GRIN2A, TPP1, GLB1, ASM, ARSA, GALC, HEXA, HEXB, GBA, or MECP2.

Compounds (e.g., small molecule payloads) described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses compounds as individual isomers substantially free of other isomers, and alternatively, as mixtures of various isomers.

### Gene Therapy Payloads

In some embodiments, gene therapy payloads (e.g., nucleic acids encoding biologically active or otherwise therapeutic molecules) are useful in the treatment of various CNS diseases and disorders. For example, gene therapies may be useful to modulate the expression or activity of various genes involved in CNS diseases and disorder, such as by encoding a protein involved in the CNS disease or disorder. In one non-limiting example, a gene therapy payload that encodes a particular biological molecule (e.g., a protein or nucleic acid) may be useful in the treatment of a CNS disease or disorder involving that particular biological molecule (e.g., a disease or disorder whose etiology involves abnormally low expression of the biological molecule or expression of an inactive form of the biological molecule). Gene therapies may be used to treat various CNS diseases and disorders, for example, by facilitating delivery of the gene therapy payload into cells of the CNS. In some embodiments, gene therapies disclosed herein can be delivered into cells of the CNS using complexes disclosed here (e.g., anti-TfR1 antibody complexes comprising the gene therapy payload).

In some embodiments, gene therapies are useful in the treatment of a neuromuscular disease or disorder (e.g., Duchenne muscular dystrophy, myotonic dystrophy, Friedreich's ataxia, or spinal muscular atrophy); amyotrophic lateral sclerosis; Parkinson's disease; Huntington's disease; Alzheimer's disease; epilepsy; a pain disorder; glycogen synthesis disorders; neurodegeneration; small fiber neuropathy; nociception-related phenotypes; Alexander disease; Angelman Syndrome; autism-spectrum disorders; retinitis pigmentosa; isolated macular dystrophy; and/or multiple sclerosis.

In some embodiments, gene therapies are useful in the treatment of essential tremor and/or hereditary dystonia.

In some embodiments, gene therapies are useful in the treatment of spinocerebellar ataxia, motor neuron disease, Dravet syndrome, Batten disease, GM1 gangliosidosis, Niemann-Pick Type A, metachromatic leukodystrophy, Krabbe disease, Tay-Sachs, Sandhoff disease, Gaucher disease types II and III, and/or Rett syndrome.

In some embodiments, gene therapies are useful in the modulation of one or more genes associated with a CNS disease or disorder. In some embodiments, the one or more genes associated with a CNS disease or disorder is DMPK, DMD, SMN, FXN, SOD1, C9orf72, ATXN2, FUS, LRRK2, SNCA, HTT, MSH3, TREM2, APOE, MAPT, APP, GYS1, PrP, VLA-4, GFAP, UBE3A, LSD, SCN9A, SCN1A, SCN2A, SCN8A, CLN3, GRIA1, or PCDH19. In some embodiments, the one or more genes associated with a CNS disease or disorder is TOR1A, THAP1, ANO3, GNAL, KMT2B, GCH1, TH, SPR, TAF1, PRKRA, ATP1A3, SGCE, PNKD, PRRT2, SLC2A1, or ECHS1. In some embodiments, the one or more genes associated with a CNS disease or disorder is PIKFYVE, SYF2, UNC13A, ATXN1, ATXN3, GRN, GRIN2A, TPP1, GLB1, ASM, ARSA, GALC, HEXA, HEXB, GBA, or MECP2.

### Molecular Payloads for the Treatment of ALS

Various molecular payloads may be useful in the treatment of ALS, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of ALS may include, in some embodiments, molecular payloads which modulate (e.g., increase or decrease) expression or activity of SOD1, ATXN2, C9orf72, and/or FUS.

Examples of oligonucleotides useful for the treatment of ALS, e.g., oligonucleotides targeting (e.g., directly or indirectly modulating the expression or activity of) genes associated with ALS (e.g., SOD1, ATXN2, C9orf72, FUS, etc.), include those listed in Table 5 below. Each oligonucleotide provided in Table 5 may have any modification pattern disclosed herein.

TABLE 5
Oligonucleotides for the treatment of ALS

| SEQ | | | |
|---|---|---|---|
| ID | Target | Oligonucleotide | |
| NO: | gene | Sequence (5′-3′) | Oligonucleotide Structure (5′-3′) |
| 22 | SOD1 | CAGGATACATTTCTACAGCT | mCes Aeo Ges Geo Aes Tds Ads mCds Ads |
| | | | Tds Tds Tds mCds Tds Ads mCeo Aes Geo |
| | | | mCes Te |
| 23 | SOD1 | TTAATGTTTATCAGGAT | Tes Teo Aeo Aes Tds Gds Tds Tds Tds Ads |
| | | | Tds mCds Ako Gko Ges Aes Te |
| 24 | SOD1 | GGATACATTTCTACAGC | Ges Geo Aeo Teo Ads mCds Ads Tds Tds |
| | | | Tds mCds Tds Ads mCko Aks Ges mCe |
| 25 | SOD1 | GGATACATTTCTACAGC | Ges Geo Aeo Teo Aes mCds Ads Tds Tds |
| | | | Tds mCds Tds Ads mCko Aks Ges mCe |
| 26 | SOD1 | GGATACATTTCTACAGC | Ges Geo Aeo Teo Aks mCds Ads Tds Tds |
| | | | Tds mCds Tds Ads mCko Aes Ges mCe |
| 27 | SOD1 | AGTGTTTAATGTTTATC | Aes Gko Teo Gks Tds Tds Tds Ads Ads Tds |
| | | | Gds Tds Tko Teo Aks Tes mCe |
| 28 | SOD1 | AGTGTTTAATGTTTATC | Aes Gko Teo Gks Tds Tds Tds Ads Ads Tds |
| | | | Gds Tds Teo Teo Aes Tes mCe |
| 29 | SOD1 | AGTGTTTAATGTTTATC | Aes Geo Tko Gks Tds Tds Tds Ads Ads Tds |
| | | | Gds Tds Teo Teo Aes Tes mCe |
| 30 | ATXN2 | TGGATTCTGTACTTTTCTCA | Tes Geo Geo Aeo Teo Teo mCds Tds Gds |
| | | | Tds Ads mCds Tds Tds Tds Tds mCeo Tes |
| | | | mCes Ae |
| 31 | ATXN2 | GTACTTTTCTCATGTGCGGC | Ges Teo Aeo mCeo Teo Tds Tds Tds mCds |
| | | | Tds mCds Ads Tds Gds Tds Geo mCeo Ges |
| | | | Ges mCe |
| 32 | ATXN2 | CTTCACATTTCGATCCAACA | Ces Teo Teo Ceo Aeo Cds Ads Tds Tds Tds |
| | | | Cds Gds Ads Tds Cds Ceo Aeo Aes Ces Ae |
| 33 | ATXN2 | GCTAACTGGTTTGCCCTTGC | Ges Ces Tes Aes Aes Cds Tds Gds Gds Tds |
| | | | Tds Tds Gds Cds Cds Ces Tes Tes Ges Ce |
| 34 | ATXN2 | CCCAAAGGGTTAATTAGGAT | Ces Ces Ces Aes Aes Ads Gds Gds Gds Tds |
| | | | Tds Ads Ads Tds Tds Aes Ges Ges Aes Te |
| 35 | ATXN2 | CTCTCCATTATTTCTTCACG | Ces Tes Ces Tes Ces Cds Ads Tds Tds Ads |
| | | | Tds Tds Tds Cds Tds Tes Ces Aes Ces Ge |
| 36 | ATXN2 | CTGGTACAGTTGCTGCTGCT | Ces Tes Ges Ges Tes Ads Cds Ads Gds Tds |
| | | | Tds Gds Cds Tds Gds Ces Tes Ges Ces Te |
| 37 | ATXN2 | GGAGCTGGAGAACCATGAGC | Ges Ges Aes Ges Ces Tds Gds Gds Ads Gds |
| | | | Ads Ads Cds Cds Ads Tes Ges Aes Ges Ce |
| 38 | ATXN2 | TGCATAGATTCCATCAAAAG | Tes Ges Ces Aes Tes Ads Gds Ads Tds Tds |
| | | | Cds Cds Ads Tds Cds Aes Aes Aes Aes Ge |
| 39 | ATXN2 | GTGGGATACAAATTCTAGGC | Ges Tes Ges Ges Ges Ads Tds Ads Cds Asd |
| | | | Ads Ads Tds Tds Cds Tes Aes Ges Ges Ce |
| 40 | ATXN2 | CCCATACGCGGTGAATTCTG | Ces Ces Ces Aes Tes Ads Cds Gds Cds Gds |
| | | | Gds Tds Gds Ads Ads Tes Tes Ces Tes Ge |
| 41 | ATXN2 | CTGCTAACTGGTTTGCCCTT | mCes Teo Geo mCeo Tds Ads Ads mCds Tds |
| | | | Gds Gds Tds Tds Tds Geo mCeo mCeo mCes |
| | | | Tes Te |
| 42 | ATXN2 | TGTACTTCACATTTGGAGCC | Tes Geo Teo Aeo mCeo Teo Tds mCds Ads |
| | | | mCds Ads Tds Tds Tds Gds Gds Aeo Ges |
| | | | mCes mCe |
| 43 | ATXN2 | CCTATCATCATTTTCCAGGG | mCes mCeo Teo Aeo Teo mCeo Ads Tds mCds |
| | | | Ads Tds Tds Tds Tds mCds mCds Aeo Ges |
| | | | Ges Ge |
| 44 | ATXN2 | TCTGTACTTTTCTCATGTGC | Tes mCeo Teo Geo Tes Ads mCds Tds Tds |
| | | | Tds Tds mCds Tds mCds Ads Teo Geo Tes |
| | | | Ges mCe |
| 45 | C9orf72 | GCCTTACTCTAGGACCAA | Ges mCeo mCeo Tes Tds Ads mCds Tds mCds |
| | | | Tds Ads Gds Geo Aeo mCeo mCes Aes Ae |
| 46 | C9orf72 | GGCCCCGGCCCCGGCC | Ge Ge Ce Cd Cd Cd Gd Gd Cd Cd Cd Cd Gd |
| | | | Ge Ce Ce |
| 47 | C9orf72 | CCGGCCCCGGCCCCGGCCCC | Ce Ce Ge Ge Ce Cd Cd Cd Gd Gd Cd Cd Cd |
| | | | Cd Gd Ge Ce Ce Ce Ce |
| 48 | C9orf72 | GGCCCCGGCCCCGGCCCCG | Ge Ge Ce Ce Ce Cd Gd Gd Cd Cd Cd Cd Gd |
| | | | Gd Cd Ce Ce Ce Ge |
| 49 | C9orf72 | GCCTTACTCTAGGACCAAGA | Ge Ce Ce Te Te Ad Cd Td Cd Td Ad Gd Gd |
| | | | Ad Cd Ce Ae Ae Ge Ae |
| 50 | FUS | GTTTATCTGAATTCGCCATA | Ges Teo Teo Teo Aeo Tds mCds Tds Gds |
| | | | Ads Ads Tds Tds mCds Gds mCeo mCeo Aes |
| | | | Tes Ae |
| 51 | FUS | GCAATGTCACCTTTCATACC | Ges Ceo Aeo Aeo Teo Gds Tds mCds Ads |
| | | | mCds mCds Tds Tds Tds Cds Aeo Teo Aes |
| | | | mCes mCe |
| 52 | FUS | GCAATGTCACCTTTCATACC | Ges mCeo Aeo Aeo Teo Gds Tds mCds Ads |
| | | | mCds mCds Tds Tds Tds Cds Aeo Teo Aes |
| | | | mCes mCe |

Abbreviations used in Table 5:
A = adenine;
mC = 5-methylcytosine;
G = guanine;
T = thymine;
e = 2′-O-methoxyethylribose modified sugar;
d = 2′-deoxyribose sugar;
s = phosphorothioate internucleoside linkage;
o = phosphodiester internucleoside linkage;
k = cEt modified sugar.
In each sequence listed in Table 5, each T may be optionally and independently replaced with a U.

Examples of oligonucleotides useful for the treatment of ALS, e.g., oligonucleotides targeting (e.g., directly or indirectly modulating the expression or activity of) genes associated with ALS (e.g., PIKFYVE, SYF2, UNC13A, etc.), include those listed in Table 6 below. Each oligonucleotide provided in Table 6 may have an modification pattern disclosed herein.

TABLE 6
Oligonucleotides for the treatment of ALS

| SEQ | | | |
|---|---|---|---|
| ID | Target | Oligonucleotide | Optional Oligonucleotide Structure* |
| NO: | Gene | Sequence (5′-3′) | (5′-3′) |
| 876 | PIKFYVE | ATGGCATGATCCCCATAAG | eseoeoeoeodsdsdsdsdsdsdsdsdsdseoeoes |
| | | C | ese |
| 877 | PIKFYVE | AGGACCAGTACAACCTGTA | eseoeoeoeodsdsdsdsdsdsdsdsdsdseoeoes |
| | | G | ese |
| 878 | PIKFYVE | AGGTGCCCCAACTTGTTAC | eseoeoeoeodsdsdsdsdsdsdsdsdsdseoeoes |
| | | C | ese |
| 879 | PIKFYVE | ACTAAGGTGCCCCAACTTG | eseoeoeoeodsdsdsdsdsdsdsdsdsdseoeoes |
| | | T | ese |
| 880 | PIKFYVE | ACCAGGTAGTCCCAAATAA | eseoeoeoeodsdsdsdsdsdsdsdsdsdseoeoes |
| | | C | ese |
| 881 | PIKFYVE | GCCTGACCCCTATACTTGA | eseoeoeoeodsdsdsdsdsdsdsdsdsdseoeoes |
| | | C | ese |
| 882 | PIKFYVE | CTGACCCCTATACTTGACA | eseoeoeoeodsdsdsdsdsdsdsdsdsdseoeoes |
| | | G | ese |
| 883 | PIKFYVE | TTAAGCCCTTTGGTCCAAC | eseoeoeoeodsdsdsdsdsdsdsdsdsdseoeoes |
| | | C | ese |
| 884 | PIKFYVE | GAGTTAGCACCCTCTACTA | eseoeoeoeodsdsdsdsdsdsdsdsdsdseoeoes |
| | | C | ese |
| 885 | PIKFYVE | GTCATAAGTCCTTGGTCAA | eseoeoeoeodsdsdsdsdsdsdsdsdsdseoeoes |
| | | C | ese |
| 886 | PIKFYVE | AAGCCTGACCCCTATACTT | eseoeoeoeodsdsdsdsdsdsdsdsdsdseoeoes |
| | | G | ese |
| 887 | PIKFYVE | AGCCTGACCCCTATACTTG | eseoeoeoeodsdsdsdsdsdsdsdsdsdseoeoes |
| | | A | ese |
| 888 | PIKFYVE | CATCCTATTAGCTTAAACC | eseoeoeoeodsdsdsdsdsdsdsdsdsdseoeoes |
| | | C | ese |
| 889 | PIKFYVE | CCTGACCCCTATACTTGAC | eseoeoeoeodsdsdsdsdsdsdsdsdsdseoeoes |
| | | A | ese |
| 890 | PIKFYVE | TATGATCTGATAGCCAACT | eseoeoeoeodsdsdsdsdsdsdsdsdsdseoeoes |
| | | C | ese |
| 891 | PIKFYVE | GTACCAGGACCAGTACAAC | eseoeoeoeodsdsdsdsdsdsdsdsdsdseoeoes |
| | | C | ese |
| 892 | PIKFYVE | CAGGACCAGTACAACCTGT | eseoeoeoeodsdsdsdsdsdsdsdsdsdseoeoes |
| | | A | ese |
| 893 | PIKFYVE | ACACCCTTTGGAGTGTCIA | eseoeoeoeodsdsdsdsdsdsdsdsdsdseoeoes |
| | | G | ese |
| 894 | PIKFYVE | GCCCCAACTTGTTACCTAA | eseoeoeoeodsdsdsdsdsdsdsdsdsdseoeoes |
| | | G | ese |
| 895 | PIKFYVE | CCCCAATCTAGTTCAAGCA | eseoeoeoeodsdsdsdsdsdsdsdsdsdseoeoes |
| | | T | ese |
| 896 | PIKFYVE | TCCAAGAGTTAGCACCCTC | eseoeoeoeodsdsdsdsdsdsdsdsdsdseoeoes |
| | | T | ese |
| 897 | PIKFYVE | GGACCAGTACAACCTGTAG | eseoeoeoeodsdsdsdsdsdsdsdsdsdseoeoes |
| | | T | ese |
| 898 | PIKFYVE | AAACCCAGTGTAGCTCCAT | eseoeoeoeodsdsdsdsdsdsdsdsdsdseoeoes |
| | | T | ese |
| 899 | PIKFYVE | TGCCCCAACTTGTTACCTA | eseoeoeoeodsdsdsdsdsdsdsdsdsdseoeoes |
| | | A | ese |
| 900 | PIKFYVE | CCAATAGCAAGCAGCCTAT | eseoeoeoeodsdsdsdsdsdsdsdsdsdseoeoes |
| | | A | ese |
| 901 | PIKFYVE | CTACAATCAAATCCTGGTA | eseoeoeoeodsdsdsdsdsdsdsdsdsdseoeoes |
| | | G | ese |
| 902 | PIKFYVE | GACCAAGTTTATGGACCCC | eseoeoeoeodsdsdsdsdsdsdsdsdsdseoeoes |
| | | A | ese |
| 903 | PIKFYVE | GTGACCACCATCTATAGTT | eseoeoeoeodsdsdsdsdsdsdsdsdsdseoeoes |
| | | A | ese |
| 904 | PIKFYVE | CTAAGGTAACTGTTCCTAG | eseoeoeoeodsdsdsdsdsdsdsdsdsdseoeoes |
| | | A | ese |
| 905 | PIKFYVE | TGACCAAGTTTATGGACCC | eseoeoeoeodsdsdsdsdsdsdsdsdsdseoeoes |
| | | C | ese |
| 906 | PIKFYVE | ACCAAGTTTATGGACCCCA | eseoeoeoeodsdsdsdsdsdsdsdsdsdseoeoes |
| | | A | ese |
| 907 | PIKFYVE | GCAATAAAGCTAACCACAT | eseoeoeoeodsdsdsdsdsdsdsdsdsdseoeoes |
| | | C | ese |
| 908 | PIKFYVE | GATTCTACCACACAGTACA | eseoeoeoeodsdsdsdsdsdsdsdsdsdseoeoes |
| | | C | ese |
| 909 | UNC13A | GCAGACAGGTGTACTGGA | esesesesesesesesesesesesesesesesese |
| 910 | UNC13A | AGACAGGTGTACTGGACA | esesesesesesesesesesesesesesesesese |
| 911 | UNC13A | ACAGGTGTACTGGACATG | esesesesesesesesesesesesesesesesese |
| 912 | UNC13A | AGGTGTACTGGACATGGT | esesesesesesesesesesesesesesesesese |
| 913 | UNC13A | AGGCAGCTGGAAGAGACA | esesesesesesesesesesesesesesesesese |
| 914 | UNC13A | GGCAGCTGGAAGAGACAT | esesesesesesesesesesesesesesesesese |
| 915 | UNC13A | GCAGCTGGAAGAGACATA | esesesesesesesesesesesesesesesesese |
| 916 | UNC13A | CAGCTGGAAGAGACATAC | esesesesesesesesesesesesesesesesese |
| 917 | UNC13A | AGCTGGAAGAGACATACC | esesesesesesesesesesesesesesesesese |
| 918 | UNC13A | GCTGGAAGAGACATACCC | esesesesesesesesesesesesesesesesese |
| 919 | UNC13A | CTGGAAGAGACATACCCA | esesesesesesesesesesesesesesesesese |
| 920 | UNC13A | TGGAAGAGACATACCCAG | esesesesesesesesesesesesesesesesese |
| 921 | UNC13A | GGAAGAGACATACCCAGA | esesesesesesesesesesesesesesesesese |
| 922 | UNC13A | GAAGAGACATACCCAGAC | esesesesesesesesesesesesesesesesese |
| 923 | UNC13A | AAGAGACATACCCAGACA | esesesesesesesesesesesesesesesesese |
| 924 | UNC13A | AGAGACATACCCAGACAC | esesesesesesesesesesesesesesesesese |
| 925 | UNC13A | GAGACATACCCAGACACA | esesesesesesesesesesesesesesesesese |
| 926 | UNC13A | AGACATACCCAGACACAA | esesesesesesesesesesesesesesesesese |
| 927 | UNC13A | GACATACCCAGACACAAA | esesesesesesesesesesesesesesesesese |
| 928 | UNC13A | ACATACCCAGACACAAAC | esesesesesesesesesesesesesesesesese |
| 929 | UNC13A | CATACCCAGACACAAACG | esesesesesesesesesesesesesesesesese |
| 930 | UNC13A | TAGTTCCTGGGGATAAGA | esesesesesesesesesesesesesesesesese |
| 931 | UNC13A | AGTTCCTGGGGATAAGAG | esesesesesesesesesesesesesesesesese |
| 932 | UNC13A | GTTCCTGGGGATAAGAGT | esesesesesesesesesesesesesesesesese |
| 933 | UNC13A | TTCCTGGGGATAAGAGTT | esesesesesesesesesesesesesesesesese |
| 934 | UNC13A | TCCTGGGGATAAGAGTTC | esesesesesesesesesesesesesesesesese |
| 935 | UNC13A | CCTGGGGATAAGAGTTCT | esesesesesesesesesesesesesesesesese |
| 936 | UNC13A | CTGGGGATAAGAGTTCTT | esesesesesesesesesesesesesesesesese |
| 937 | UNC13A | TGGGGATAAGAGTTCTTT | esesesesesesesesesesesesesesesesese |
| 938 | UNC13A | GGGGATAAGAGTTCTTTC | esesesesesesesesesesesesesesesesese |
| 939 | UNC13A | GGGATAAGAGTTCTTTCC | esesesesesesesesesesesesesesesesese |
| 940 | UNC13A | CATCTGTTCAATCATTCA | esesesesesesesesesesesesesesesesese |
| 941 | UNC13A | TCTGTTCAATCATTCATT | esesesesesesesesesesesesesesesesese |
| 942 | UNC13A | TGTTCAATCATTCATTCA | esesesesesesesesesesesesesesesesese |
| 943 | UNC13A | TTCAATCATTCATTCATT | esesesesesesesesesesesesesesesesese |
| 944 | UNC13A | CAATCATTCATTCATTCA | esesesesesesesesesesesesesesesesese |
| 945 | UNC13A | ATCATTCATTCATTCACC | esesesesesesesesesesesesesesesesese |
| 946 | UNC13A | CATTCATTCATTCACCAG | esesesesesesesesesesesesesesesesese |
| 947 | UNC13A | TTCATTCATTCACCAGCA | esesesesesesesesesesesesesesesesese |
| 948 | UNC13A | CATTCATTCACCAGCATT | esesesesesesesesesesesesesesesesese |
| 949 | UNC13A | TTCATTCACCAGCATTTA | esesesesesesesesesesesesesesesesese |
| 950 | UNC13A | CATTCACCAGCATTTATT | esesesesesesesesesesesesesesesesese |
| 951 | UNC13A | TTCACCAGCATTTATTCA | esesesesesesesesesesesesesesesesese |
| 952 | UNC13A | CACCAGCATTTATTCAAC | esesesesesesesesesesesesesesesesese |
| 953 | UNC13A | CCAGCATTTATTCAACAA | esesesesesesesesesesesesesesesesese |
| 954 | UNC13A | AGCATTTATTCAACAAAC | esesesesesesesesesesesesesesesesese |
| 955 | UNC13A | CATTTATTCAACAAACTA | esesesesesesesesesesesesesesesesese |
| 956 | UNC13A | TTTATTCAACAAACTAGT | esesesesesesesesesesesesesesesesese |
| 957 | UNC13A | TATTCAACAAACTAGTTC | esesesesesesesesesesesesesesesesese |
| 958 | UNC13A | TTCAACAAACTAGTTCCT | esesesesesesesesesesesesesesesesese |
| 959 | UNC13A | CAACAAACTAGTTCCTGG | esesesesesesesesesesesesesesesesese |
| 960 | UNC13A | ACAAACTAGTTCCTGGGG | esesesesesesesesesesesesesesesesese |
| 961 | UNC13A | AAACTAGTTCCTGGGGAT | esesesesesesesesesesesesesesesesese |
| 962 | UNC13A | ACTAGTTCCTGGGGATAA | esesesesesesesesesesesesesesesesese |
| 963 | UNC13A | GGATAAGAGTTCTTTCCA | esesesesesesesesesesesesesesesesese |
| 964 | UNC13A | GATAAGAGTTCTTTCCAG | esesesesesesesesesesesesesesesesese |
| 965 | UNC13A | ATAAGAGTTCTTTCCAGG | esesesesesesesesesesesesesesesesese |
| 966 | UNC13A | TAAGAGTTCTTTCCAGGA | esesesesesesesesesesesesesesesesese |
| 967 | UNC13A | AAGAGTTCTTTCCAGGAA | esesesesesesesesesesesesesesesesese |
| 968 | UNC13A | AGAGTTCTTTCCAGGAAA | esesesesesesesesesesesesesesesesese |
| 969 | UNC13A | GAGTTCTTTCCAGGAAAC | esesesesesesesesesesesesesesesesese |
| 970 | UNC13A | AGTTCTTTCCAGGAAACC | esesesesesesesesesesesesesesesesese |
| 971 | UNC13A | GTTCTTTCCAGGAAACCC | esesesesesesesesesesesesesesesesese |
| 972 | UNC13A | ACTCATCACTCATTCATC | esesesesesesesesesesesesesesesesese |
| 973 | UNC13A | TCATCACTCATTCATCTG | esesesesesesesesesesesesesesesesese |
| 974 | UNC13A | ATCACTCATTCATCTGTT | esesesesesesesesesesesesesesesesese |
| 975 | UNC13A | CACTCATTCATCTGTTCA | esesesesesesesesesesesesesesesesese |
| 976 | UNC13A | CTCATTCATCTGTTCAAT | esesesesesesesesesesesesesesesesese |
| 977 | UNC13A | CATTCATCTGTTCAATCA | esesesesesesesesesesesesesesesesese |
| 978 | UNC13A | TTCATCTGTTCAATCATT | esesesesesesesesesesesesesesesesese |
| 979 | UNC13A | CACCCATCTCTCCATCCAT | LsdsdsLsdsdsLsdsdsLsdsdsLsdsdsLsdsds |
| | | GC | Lsdsd |
| 980 | UNC13A | CACCCATCTCTCCATCCAT | LsdsdsLsdsdsLsdsdsLsdsdsLsdsdsLsdsds |
| | | CC | Lsdsd |
| 981 | UNC13A | CTCACCCATCTCTCCATCC | LsdsdsLsdsdsLsdsdsLsdsdsLsdsdsLsdsds |
| | | AT | LsdsL |
| 982 | UNC13A | TGTACTCACCCATCTCTCC | LsdsdsLsdsdsLsdsdsLsdsdsLsdsdsLsdsds |
| | | AT | LsdsL |
| 983 | UNC13A | TCCATGTACTCACCCATCT | LsdsdsLsdsdsLsdsdsLsdsdsLsdsdsLsdsds |
| | | CT | LsdsL |
| 984 | UNC13A | TCCATCCATGTACTCACCC | LsdsdsLsdsdsLsdsdsLsdsdsLsdsdsLsdsds |
| | | AT | LsdsL |
| 985 | UNC13A | ATCTATCCATCCATGTACT | LsdsdsLsdsdsLsdsdsLsdsdsLsdsdsLsdsds |
| | | CA | LsdsL |
| 986 | UNC13A | TCACCCATCTCTCCATC | LsdsdsLsdsdsLsdsdsLsdsdsLsdsdsLsL |
| 986 | UNC13A | TCACCCATCTCTCCATC | LsdsLsdsLsdsLsdsLsdsLsdsLsdsLSdsL |
| 987 | UNC13A | GTACTCACCCATCTCTC | LsdsdsLsdsdsLsdsdsLsdsdsLsdsdsLsL |
| 987 | UNC13A | GTACTCACCCATCTCTC | LsdsLsdsLsdsLsdsLsdsLsdsLsdsLsdsL |
| 988 | UNC13A | CACCCATCTCTCC | dsLsdsLsdsLsdsLsdsLsdsLsd |
| 989 | UNC13A | CAUGUACUCACCCAUCUCU | msmsmsmsmsmsmsmsmsmsmsmsmsmsmsmsmsms |
| | | CCAUCC | msmsmsmsmsmsm |
| 990 | UNC13A | AUCCAUGUACUCACCCAUC | msmsmsmsmsmsmsmsmsmsmsmsmsmsmsmsmsms |
| | | UCUCCAUCCAU | msmsmsmsmsmsmsmsmsmsmsm |
| 991 | UNC13A | CAGGCAGCTGGAAGAGACA | LsdsdsLsdsdsLsdsdsLsdsdsLsdsdsLsdsds |
| | | TA | LsdsL |
| 992 | UNC13A | AACCCAGGCAGCTGGAAGA | LsdsdsLsdsdsLsdsdsLsdsdsLsdsdsLsdsds |
| | | GA | LsdsL |
| 993 | UNC13A | GGAAACCCAGGCAGCTGGA | LsdsdsLsdsdsLsdsdsLsdsdsLsdsdsLsdsds |
| | | AG | LsdsL |
| 994 | UNC13A | TAGGGCTGGAATAGAAGGA | LsdsdsLsdsdsLsdsdsLsdsdsLsdsdsLsdsds |
| | | AG | LsdsL |
| 995 | UNC13A | TGAGTGGTTAGGGCTGGAA | LsdsdsLsdsdsLsdsdsLsdsdsLsdsdsLsdsds |
| | | TA | LsdsL |

*e = 2′-MOE modified sugar;
L = locked nucleic acid (LNA) nucleoside;
d = deoxyribonucleoside;
m = 2′-O-methyl ribonucleoside;
s = phosphorothioate internucleoside linkage.
In each sequence listed in Table 6, each T may be optionally and independently replaced with a U, and each U may be optionally and independently replaced with a T.

Examples of small molecules useful for the treatment of ALS include:

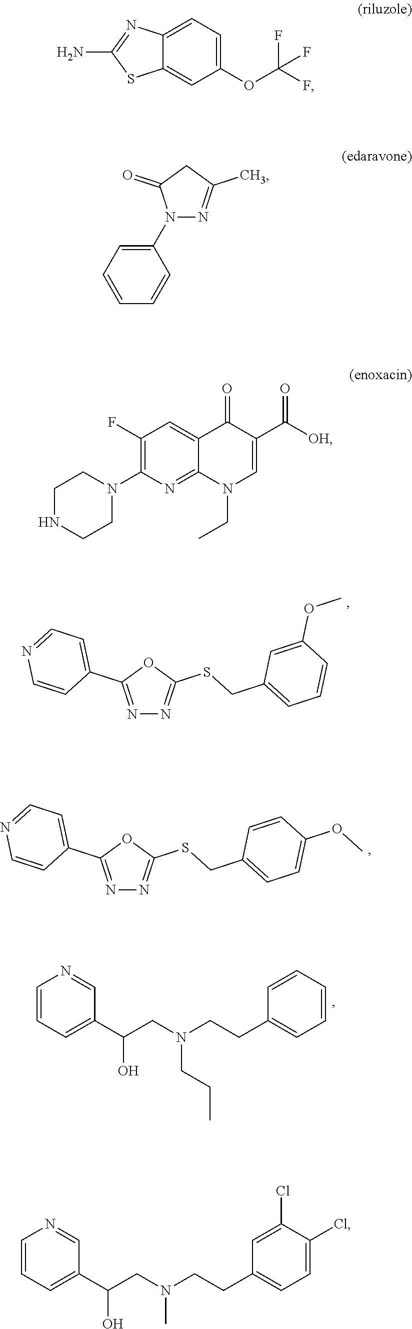

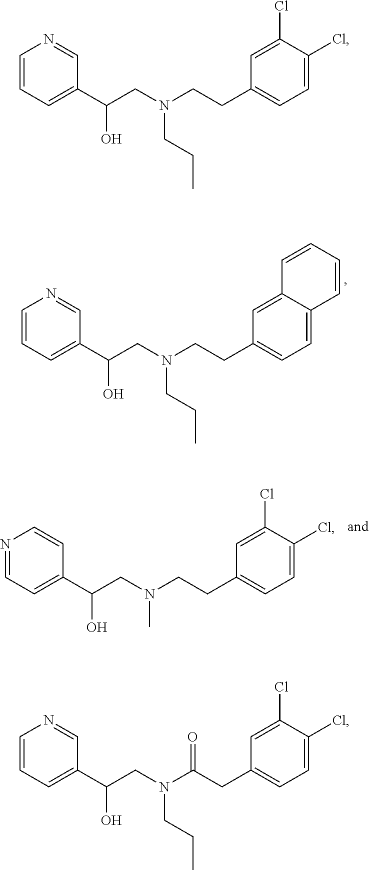 [SMILES: CCCN(CCc1ccc(Cl)c(Cl)c1)CC(O)c1cccnc1 ; CCCN(CCc1ccc2ccccc2c1)CC(O)c1cccnc1 ; CN(CCc1ccc(Cl)c(Cl)c1)CC(O)c1ccncc1 ; CCCN(CC(O)c1cccnc1)C(=O)Cc1ccc(Cl)c(Cl)c1]

and pharmaceutically acceptable salts, co-crystals, tautomers, stereoisomers, solvates, hydrates, polymorphs, isotopically enriched compounds, and prodrugs thereof. Additional examples of small molecules useful for the treatment of ALS include:

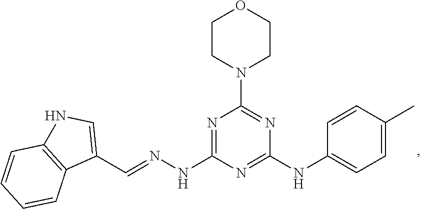 [SMILES: Cc1ccc(Nc2nc(N/N=C/c3c[nH]c4ccccc34)nc(N3CCOCC3)n2)cc1]

apilimod, APY0201, YM-201636, and pharmaceutically acceptable salts, co-crystals, tautomers, stereoisomers, solvates, hydrates, polymorphs, isotopically enriched compounds, and prodrugs thereof.

Examples of polypeptides useful for the treatment of ALS include (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide.

### Molecular Payloads Targeting SOD1

The superoxide dismutase 1 (SOD1) gene, and mutations therein, are implicated in ALS, which predominantly affects upper and lower motor neurons. Modulation of SOD1 expression and activity (e.g., by suppressing the expression and/or activity of mutant SOD1 protein) therefore in some embodiments can have a therapeutic effect in subjects with ALS.

### Oligonucleotides

SOD1 expression and/or activity in some embodiments can be modulated by the use of oligonucleotides targeting SOD1 sequences.

In some embodiments, an oligonucleotide useful for the treatment of ALS, e.g., targeting (e.g., directly or indirectly modulating the expression or activity of) SOD1, comprises a region of complementarity to a SOD1 transcript provided in Table 3, e.g., provided by SEQ ID NO: 392.

In some embodiments, examples of oligonucleotides useful for the treatment of ALS, e.g., targeting (e.g., directly or indirectly modulating the expression or activity of) SOD1, are provided in Smith, et al., “Antisense oligonucleotide therapy for neurodegenerative disease” J. Clin. Invest. (2006) 116(8): 2290-96 doi:10.1172/JCI25424; van Zundert, et al., “Silencing strategies for therapy of SOD1-mediated ALS” Neurosis. Lett. (2017) 636:32-39 doi:10.1016/j.neulet.2016.07.059; US Patent Application Publication No. 20040091919A1, published on May 13, 2004, entitled “Antisense Modulation of Superoxide Dismutase 1, Soluble Expression”; US Patent Application Publication No. 20090306005A1, published Dec. 10, 2009, entitled “Compounds and methods for modulating expression of PCSK9”; US Patent Application Publication No. 20140378533A1, published Dec. 25, 2014, entitled “Modulation of RNA by repeat targeting”; US Patent Application Publication No. 20150184154A1, published Jul. 2, 2015, entitled “New Treatment for Neurodegenerative Diseases”; US Patent Application Publication No. 20160272976A1, published Sep. 22, 2016, entitled “Products and Methods for Treatment of Familial Amyotrophic Lateral Sclerosis”; US Patent Application Publication No. 20160222391A1, published Aug. 4, 2016, entitled “Compositions and Methods for Treating Amyotrophic Lateral Sclerosis”; US Patent Application Publication No. 20170037399A1, published Feb. 9, 2017, entitled “Chiral Design”; US Patent Application Publication No. 20170037410A1, published Feb. 9, 2017, entitled “Compositions for Modulating SOD-1 Expression”; US Patent Application Publication No. 20170152517A1, published Jun. 1, 2017, entitled “Treatment of Amyotrophic Lateral Sclerosis”; US Patent Application Publication No. 20160089453A1, published Mar. 31, 2016, entitled “RNA-Modulating Agents”; US Patent Application Publication No. 20180282732A1, published Oct. 4, 2018, entitled “Compositions and Methods of Treating Amyotrophic Lateral Sclerosis (ALS)”; International Patent Application Publication No. WO2016180784A1, published Nov. 17, 2016, entitled “Improved Treatments Using Oligonucleotides”; US Patent Application Publication No. 20180161357A1, published Jun. 14, 2018, entitled “MIR-155 Inhibitors for Treating Amyotrophic Lateral Sclerosis (ALS)”; US Patent Application Publication No. 20180195072A1, published Jul. 12, 2018, entitled “Nucleic acid molecules targeting superoxide dismutase 1 (sod1)”; US Patent Application Publication No. 20180216107A1, published Aug. 2, 2018, entitled “Oligonucleotide compositions and methods thereof”; US Patent Application Publication No. 20210228615A1, published Jul. 29, 2021, entitled “Oligonucleotide compositions and methods thereof”; US Patent Application Publication No. 20190167815A1, published Jun. 6, 2019, entitled “Methods and compositions for the treatment of rare diseases”; US Patent Application Publication No. 20210054383A1, published Feb. 25, 2021, entitled “Oligonucleotides for modulating tmem106b expression”; US Patent Application Publication No. 20210269881A1, published Sep. 2, 2021, entitled “Long non-coding RNAs (lncRNAs) for the diagnosis and therapeutics of brain disorders, in particular cognitive disorders”; U.S. Pat. No. 10,808,247B2, published Oct. 20, 2020, entitled “Methods for treating neurological disorders using a synergistic small molecule and nucleic acids therapeutic approach”; U.S. Pat. No. 11,118,179B2, published Sep. 14, 2021, entitled “Mixed tricyclo-DNA, 2′-modified RNA oligonucleotide compositions and uses thereof”; International Patent Application Publication No. WO2020198270A1, published Oct. 1, 2020, entitled “Compositions and methods for treating neurodegenerative disorders”; US Patent Application Publication No. 20220170025A1, published Jun. 2, 2022, entitled “Compositions and methods for inhibiting gene expression in the central nervous system”; U.S. Pat. No. 10,174,328B2, published Jan. 8, 2019, entitled “Compositions and methods for treating amyotrophic lateral sclerosis”; International Patent Application Publication No. WO2020222182A1, published Nov. 5, 2020, entitled “Treatment for SOD1 associated disease”; International Patent Application Publication No. WO2020247419A2, published Dec. 10, 2020, entitled “Oligonucleotides and methods of use for treating neurological diseases”; International Patent Application Publication No. WO2021029896A1, published Feb. 18, 2021, entitled “Splice modulating oligonucleotides targeting receptor for advanced glycation end products and methods of use”; US Patent Application Publication No. 20220090036A1, published Mar. 24, 2022, entitled “Compositions and methods for the targeting of SOD1”; International Patent Application Publication No. WO2021108602A1, published Nov. 25, 2020, entitled “Methods and compositions for neuroprotection”; International Patent Application Publication No. WO2021156832A1, published Feb. 6, 2021, entitled “Use of miRNA-485 inhibitors for treating amyotrophic lateral sclerosis (ALS)”; US Patent Application Publication No. 20220073930A1, published Mar. 10, 2022, entitled “Compositions and methods for treating and preventing amyotrophic lateral sclerosis”; the entire contents of each of which are herein incorporated by reference.

Certain oligonucleotides provided in this section may be useful in treating ALS by modulating the activity of genes and/or gene products other than SOD1 genes/gene products, such as C9orf72, ATXN2, and/or FUS genes/gene products.

### Polypeptides

SOD1 expression and/or activity in some embodiments can be modulated by the use of SOD1 polypeptides or polypeptides that can interact with SOD1 (e.g., to modulate its enzymatic activity).

In some embodiments, examples of polypeptides (e.g., peptides; proteins, such as enzymes; antibodies; etc.) useful in the treatment of ALS are provided in Martin, et al. “GNX-4728, a novel small molecule drug inhibitor of mitochondrial permeability transition, is therapeutic in a mouse model of amyotrophic lateral sclerosis” Front. Cell Neurosci. 8: article 433 (2014); doi: 10.3389/fncel.2014.00433; US Patent Application Publication No. 20090124993A1, published May 14, 2009, entitled “Treating neurological disorders”; US Patent Application Publication No. 20140044722A1, published Feb. 13, 2014, entitled “Anti-SOD1 Antibodies and Uses Thereof”; US Patent Application Publication No. 20140301945, published Oct. 9, 2014, entitled “Human Anti-SOD1 Antibodies”; International Patent Application Publication No. WO2013106672A1, published Jul. 18, 2013, entitled “Methods and Compositions for the Treatment of Neurodegenerative Disease”; US Patent Application Publication No. 20150184154A1, published Jul. 2, 2015, entitled “New Treatment for Neurodegenerative Diseases”; US Patent Application Publication No. 20150259391A1, published Sep. 17, 2015, entitled “Treatment of Amyotrophic Lateral Sclerosis”; US Patent Application Publication No. 20160115245A1, published Apr. 28, 2016, entitled “Single Domain Antibodies Against SOD1 and Their Use in Medicine”; US Patent Application Publication No. 20190022179A1, published Jan. 24, 2019, entitled “Composition and method for treating amyotrophic lateral sclerosis”; US Patent Application Publication No. 20200247854A1, published Aug. 6, 2020, entitled “Pharmaceutical composition for preventing or treating neurodegenerative disease comprising nckap1 protein or gene encoding same”; US Patent Application Publication No. 20210206876A1, published Jul. 8, 2021, entitled “DPP3 binder directed to and binding to specific DPP3-epitopes and its use in the prevention or treatment of diseases/acute conditions that are associated with oxidative stress”; International Patent Application Publication No. WO2019104311A1, published May 31, 2019, entitled “Compositions and methods for suppressing neurological disease”; US Patent Application Publication No. 20210100869A1, published Apr. 8, 2021, entitled “Compositions and methods of using same for treating amyotrophic lateral sclerosis (ALS)”; US Patent Application Publication No. 20200172590A1, published Jun. 4, 2020, entitled “Methods of treating neurological diseases”; US Patent Application Publication No. 20210162002A1, published Jun. 3, 2021, entitled “Regenerating functional neurons for treatment of spinal cord injury and ALS”; U.S. Pat. No. 10,808,247B2, published Oct. 20, 2020, entitled “Methods for treating neurological disorders using a synergistic small molecule and nucleic acids therapeutic approach”; US Patent Application Publication No. 20210284702A1, published Sep. 16, 2021, entitled “Fusion proteins comprising progranulin”; US Patent Application Publication No. 20220017634A1, published Jan. 20, 2022, entitled “Engineered bispecific proteins”; US Patent Application Publication No. 20220034907A1, published Feb. 3, 2022, entitled “Neurofilament protein for guiding therapeutic intervention in amyotrophic lateral sclerosis”; the entire contents of each of which are herein incorporated by reference.

Certain polypeptides provided in this section may be useful in treating ALS by modulating the activity of genes and gene products other than SOD1 genes/gene products, such as C9orf72, ATXN2, and/or FUS genes/gene products.

### Small Molecules

SOD1 expression and/or activity in some embodiments can be modulated by the use of small molecules that can modulate SOD1 (e.g., to modulate its enzymatic activity, or its expression).

In some embodiments, examples of small molecules useful in the treatment of ALS are provided in US Patent Application Publication No. 20040219552A1, published Nov. 4, 2004, entitled “Novel Molecular Target for Neurotoxicity”; US Patent Application Publication No. 20030130357A1, published Jul. 10, 2003, entitled “Use of Polyamine Analogs for Amyotrophic Lateral Sclerosis”; US Patent Application Publication No. 20100152125A1, published Jun. 17, 2010, entitled “Compositions and Methods for the Diagnosis, Treatment, and Prevention of Amyotrophic Lateral Sclerosis and Related Neurological Diseases”; US Patent Application Publication No. 20100331417A1, published Dec. 30, 2010, entitled “Treatment of Neural Diseases or Conditions”; US Patent Application Publication No. 20110076236A1, published Mar. 31, 2011, entitled “Compositions and Methods of Treatment Using Modulators of Motoneuron Diseases”; US Patent Application Publication No. 20110166115A1, published Jul. 7, 2011, entitled “Use of Mifepristone for the Treatment of Amyotrophic Lateral Sclerosis”; US Patent Application Publication No. 20150164901A1, published Jun. 18, 2015, entitled “Compounds, Compositions and Methods for Treating or Preventing Neurodegenerative Disorders”; US Patent Application Publication No. 20160082015A1, published Mar. 24, 2016, entitled “Methods, Compositions and Kits for Promoting Motor Neuron Survival and Treating and Diagnosing Neurodegenerative Disorders”; US Patent Application Publication No. 20160287549A1, published Oct. 6, 2016, entitled “Novel Methods for Treating Neurodegenerative Diseases”; US Patent Application Publication No. 20150210679A1, published Jul. 30, 2015, entitled “Small Molecule Inhibitors of Superoxide Dismutase Expression”; International PCT Application Publication No. WO2021174167A1, published Sep. 2, 2021, entitled “Compounds and methods for modulating splicing”; US Patent Application Publication No. 20180028520A1, published Feb. 1, 2018, entitled “Methods and Pharmaceutical Compositions for Treatment of Amyotrophic Lateral Sclerosis”; International Patent Application Publication No. WO2016114655A1, published Jul. 21, 2016, entitled “Treating neuromuscular or neurologic disease through reducing gabaergic and/or glycinergic inhibitory neurotransmitter overstimulation”; US Patent Application Publication No. 20170157197A1, published Jun. 8, 2017, entitled “Methods of Using GM604 in Modulating ALS Disease Biomarkers Leading to Prognosis and Therapeutic Treatment for ALS Disease”; US Patent Application Publication No. 20170354639A1, published Dec. 14, 2017, entitled “Diterpenoid derivatives and methods of use thereof”; US Patent Application Publication No. 20180289655A1, published Oct. 11, 2018, entitled “Methods and Compositions for the Intravenous Administration of Fumarates for the Treatment of Neurological Diseases”; US Patent Application Publication No. 20170226127A1, published Aug. 10, 2017, entitled “Compound, compositions, and methods”; US Patent Application Publication No. 20170362206A1, published Jun. 15, 2017, entitled “Compound, compositions, and methods”; US Patent Application Publication No. 20180327391A1, published Nov. 15, 2018, entitled “Compound, compositions, and methods”; US Patent Application Publication No. 20190300537A1, published Oct. 3, 2019, entitled “Compound, compositions, and methods”; US Patent Application Publication No. 20190194170A1, published Jun. 27, 2019, entitled “Polymorphs and solid forms of a pyrimidinylamino-pyrazole compound, and methods of production”; U.S. Pat. No. 9,669,014B2, published Jun. 6, 2017, entitled “Small molecule inhibitors of superoxide dismutase expression”; US Patent Application Publication No. 20210130308A1, published May 6, 2021, entitled “Modulators of eukaryotic initiation factor 2”; US Patent Application Publication No. 20180353480A1, published Dec. 13, 2018, entitled “Isoxazolidine derived inhibitors of receptor interacting protein kinase 1 (RIPK1)”; US Patent Application Publication No. 20200079784A1, published Mar. 12, 2020, entitled “Compound, compositions, and methods”; US Patent Application Publication No. 20200331900A1, published Oct. 22, 2020, entitled “Compounds, compositions, and methods”; US Patent Application Publication No. 20210147435A1, published May 20, 2021, entitled “Compounds, compositions, and methods”; US Patent Application Publication No. 20210292311A1, published Sep. 23, 2021, entitled “Compounds, compositions, and methods”; US Patent Application Publication No. 20220177456A1, published Jun. 9, 2022, entitled “Compounds, compositions, and methods”; US Patent Application Publication No. 20200087319A1, published Mar. 19, 2020, entitled “Kinase Inhibitors and Uses Thereof”; US Patent Application Publication No. 20190359634A1, published Nov. 28, 2019, entitled “ASK1 inhibiting agents”; US Patent Application Publication No. 20200368267A1, published Nov. 26, 2020, entitled “Prophylactic and/or therapeutic agent for amyotrophic lateral sclerosis”; US Patent Application Publication No. 20210115020A1, published Apr. 22, 2021, entitled “ASK1 inhibiting agents”; US Patent Application Publication No. 20210317103A1, published Oct. 14, 2021, entitled “ASK1 inhibiting agents”; US Patent Application Publication No. 20210300946A1, published Sep. 30, 2021, entitled “Pyridine macrocycle compounds as ASK1 inhibiting agents”; US Patent Application Publication No. 20210353611A1, published Nov. 18, 2021, entitled “Methods of treating amyotrophic lateral sclerosis”; US Patent Application Publication No. US20210023062A1, published Jan. 28, 2021, entitled “Compositions and Methods for the Treatment of Amyotrophic Lateral Sclerosis, Parkinson's Disease, Parkinson's Disease with Dementia, Dementia with Lewy Bodies, and Multiple System Atrophy”; International Patent Application Publication No. WO2021040627A1, published Mar. 4, 2021, entitled “A method of promoting survival and/or function of a motor neuron and related agents, uses and methods”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating ALS by modulating the activity of genes and gene products other than SOD1 genes/gene products, such as C9orf72, ATXN2, and/or FUS genes/gene products.

### Gene Therapies

SOD1 expression and/or activity in some embodiments can be modulated by the use of gene therapies that can modulate SOD1 (e.g., by delivery of nucleic acids encoding SOD1 or other molecules that interact with SOD1).

In some embodiments, gene therapies, such as those involving administration of a compounds encoding useful therapeutic agents, useful in the treatment of ALS are provided in US Patent Application Publication No. 20030161814A1, published Aug. 28, 2003, entitled “Adeno-Associated Virus-Mediated Delivery of GDNF to Skeletal Muscles”; US Patent Application Publication No. 20130287736A1, published Oct. 31, 2013, entitled “Gene Therapy for Neurodegenerative Disorders”; US Patent Application Publication No. 20150182637A1, published Jul. 2, 2015, entitled “Widespread Gene Delivery of Gene Therapy Vectors”; US Patent Application Publication No. 20150259391A1, published Sep. 17, 2015, entitled “Treatment of Amyotrophic Lateral Sclerosis”; US Patent Application Publication No. 20160272976A1, published Sep. 22, 2016, entitled “Products and Methods for Treatment of Familial Amyotrophic Lateral Sclerosis”; US Patent Application Publication No. 20160130567A1, published May 12, 2016, entitled “Messenger UNA Molecules and Uses Thereof”; US Patent Application Publication No. 20180021364A1, published Jan. 25, 2018, entitled “Central Nervous System Targeting Polynucleotides”; US Patent Application Publication No. 20200297868A1, published Sep. 24, 2020, entitled “Methods and compositions for the treatment of ALS”; US Patent Application Publication No. 20180327471A1, published Nov. 15, 2018, entitled “Translatable molecules and synthesis thereof”; US Patent Application Publication No. 20200247854A1, published Aug. 6, 2020, entitled “Pharmaceutical composition for preventing or treating neurodegenerative disease comprising nckap1 protein or gene encoding same”; US Patent Application Publication No. 20210024907A1, published Jan. 28, 2021, entitled “Nucleic acid-based therapeutics”; US Patent Application Publication No. 20210254103A1, published Aug. 19, 2021, entitled “Treatment of amyotrophic lateral sclerosis and disorders associated with the spinal cord”; US Patent Application Publication No. 20200172590A1, published Jun. 4, 2020, entitled “Methods of treating neurological diseases”; US Patent Application Publication No. 20220090036A1, published Mar. 24, 2022, entitled “Compositions and methods for the targeting of SOD1”; International Patent Application Publication No. WO2021205010A1, published Oct. 14, 2021, entitled “Nucleic acids encoding human FUS protein and use in the treatment of amyotrophic lateral sclerosis (ALS)”; International Patent Application Publication No. WO2022060857A1, published Mar. 24, 2022, entitled “Compositions and methods for treating amyotrophic lateral sclerosis (ALS) with aav-miR-SOD1”; the entire contents of each of which are herein incorporated by reference.

Certain gene therapies provided in this section may be useful in treating ALS by modulating the activity of genes and gene products other than SOD1 genes/gene products, such as C9orf72, ATXN2, and/or FUS genes/gene products.

### Molecular Payloads Targeting ATXN2

The ATXN2 gene, which encodes the ataxin-2 protein, and mutations therein, are implicated in ALS, which predominantly affects upper and lower motor neurons. Modulation of ATXN2 expression and activity (e.g., by suppressing the expression and/or activity of mutant ATXN2 protein and/or its interactions with other proteins) therefore in some embodiments can have a therapeutic effect in subjects with ALS.

### Oligonucleotides

ATXN2 (and/or ataxin-2 protein encoded by ATXN2) expression and/or activity in some embodiments can be modulated by the use of oligonucleotides targeting ATXN2 sequences.

In some embodiments, an oligonucleotide useful for the treatment of ALS, e.g., targeting (e.g., directly or indirectly modulating the expression or activity of) ATXN2, comprises a region of complementarity to an ATXN2 transcript provided in Table 3, e.g., provided by any one of SEQ ID NOs: 396-400.

In some embodiments, examples of oligonucleotides useful for the treatment of ALS, e.g., targeting (e.g., directly or indirectly modulating the expression or activity of) ATXN2, are provided in Becker et al. (2017) “Therapeutic reduction of ataxin-2 extends lifespan and reduces pathology in TDP-43 mice” Nature 544:367-371; Scoles et al. (2017) “Antisense oligonucleotide therapy for spinocerebellar ataxia type 2” Nature 544:362-366; US Patent Application Publication No. 20110142789A1, published Jun. 16, 2011, entitled “Compositions and Methods for the Diagnosis and Treatment of Amyotrophic Lateral Sclerosis”; US Patent Application Publication No. 20130225659A1, published Aug. 29, 2013, entitled “Modulation of nuclear-retained RNA”; U.S. Pat. No. 1,107,486B2, published Aug. 3, 2021, entitled “Compounds and methods for reducing ATXN2 expression”; US Patent Application Publication No. 20220064639A1, published Mar. 3, 2022, entitled “Compounds and methods for reducing ATXN2 expression”; U.S. Pat. No. 10,533,178B2, published Jan. 14, 2020, entitled “Methods for modulating Ataxin 2 expression”; U.S. Pat. No. 10,006,027B2, published Jun. 26, 2018, entitled “Methods for modulating Ataxin 2 expression”; U.S. Pat. No. 10,308,934B2, published Jun. 4, 2019, entitled “Compositions for modulating Ataxin 2 expression”; U.S. Pat. No. 11,111,494B2, published Sep. 7, 2021, entitled “Compositions for modulating Ataxin 2 expression”; U.S. Pat. No. 11,345,915B2, published May 31, 2022, entitled “RNA modulating oligonucleotides with improved characteristics for the treatment of neuromuscular disorders”; US Patent Application Publication No. 20140378533A1, published Dec. 25, 2014, entitled “Modulation of RNA by repeat targeting”; US Patent Application Publication No. 20150148404A1, published May 28, 2015, entitled “RNA Modulating Oligonucleotides with Improved Characteristics for the Treatment of Neuromuscular Disorders”; US Patent Application Publication No. 20210169914A1, published Jun. 10, 2021, entitled “Nucleic acids and nucleic acid analogs for treating, preventing, and disrupting pathological polynucleotide-binding protein inclusions”; US Patent Application Publication No. 20160040163A1, published Feb. 11, 2016, entitled “DNAi for the modulation of genes”; U.S. Pat. No. 10,174,328B2, published Jan. 8, 2019, entitled “Compositions and methods for treating amyotrophic lateral sclerosis”; US Patent Application Publication No. 20220162615A1, published May 26, 2022, entitled “Methods for reducing ataxin-2 expression”; the entire contents of each of which are herein incorporated by reference.

Certain oligonucleotides provided in this section may be useful in treating ALS by modulating the activity of genes and/or gene products other than ATXN2 genes/gene products, such as C9orf72, SOD1, and/or FUS genes/gene products.

### Polypeptides

ATXN2 expression and/or activity in some embodiments can be modulated by the use of ataxin-2 polypeptides or polypeptides that can interact with ataxin-2 (e.g., to modulate its biological activity and/or its interaction with other biomolecules).

In some embodiments, examples of polypeptides (e.g., peptides; proteins, such as enzymes; antibodies; etc.) useful in the treatment of ALS are provided in US Patent Application Publication No. 20110142789A1, published Jun. 16, 2011, entitled “Compositions and Methods for the Diagnosis and Treatment of Amyotrophic Lateral Sclerosis”; U.S. Pat. No. 10,066,007B2, published Sep. 4, 2019, entitled “Dipeptide-repeat proteins as therapeutic target in neurodegenerative diseases with hexanucleotide repeat expansion”; U.S. Pat. No. 11,273,149B2, published Mar. 15, 2022, entitled “Compositions and methods for the treatment of amyotrophic lateral sclerosis, Parkinson's disease, Parkinson's disease with dementia, dementia with Lewy bodies, and multiple system atrophy”; U.S. Pat. No. 8,673,852B2, entitled “Methods of treating neuronal disorders using MNTF peptides and analogs thereof”; International PCT Application Publication No. WO2021222168A2, published Nov. 4, 2021, entitled “Compositions and methods for the treatment of tdp-43 proteinopathies”; the entire contents of each of which are herein incorporated by reference.

Certain polypeptides provided in this section may be useful in treating ALS by modulating the activity of genes and gene products other than ATXN2 genes/gene products, such as C9orf72, SOD1, and/or FUS genes/gene products.

### Small Molecules

ATXN2 expression and/or activity in some embodiments can be modulated by the use of small molecules that can modulate ATXN2 (e.g., to modulate its biological activity, its expression, and/or its interaction with other biomolecules).

In some embodiments, examples of small molecules useful in the treatment of ALS are provided in US Patent Application Publication No. 20110142789A1, published Jun. 16, 2011, entitled “Compositions and Methods for the Diagnosis and Treatment of Amyotrophic Lateral Sclerosis”; US Patent Application Publication No. 20130303562A1, published Nov. 14, 2013, entitled “Chemical and RNAi suppressors of neurotoxicity in Huntington's disease”; US Patent Application Publication No. 20140228333A1, published Mar. 29, 2016, entitled “Methods for inhibiting muscle atrophy”; International PCT Application Publication No. WO2021174167A1, published Sep. 2, 2021, entitled “Compounds and methods for modulating splicing”; International PCT Patent Application Publication No. WO2013043669A1, published Mar. 28, 2013, entitled “Peptoid compositions for the treatment of Alzheimer's disease and polyglutamine expansion disorder”; U.S. Pat. No. 10,159,670B2, published Dec. 25, 2018, entitled “Methods of diagnosing and treating motor neuron diseases and other cellular stress-related diseases”; U.S. Pat. No. 9,790,188B2, published Oct. 17, 2017, entitled “Benzimidazole derivatives and uses thereof”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating ALS by modulating the activity of genes and gene products other than ATXN2 genes/gene products, such as C9orf72, SOD1, and/or FUS genes/gene products.

### Gene Therapies

ATXN2 expression and/or activity in some embodiments can be modulated by the use of gene therapies that can modulate ATXN2 (e.g., by delivery of nucleic acids encoding ATXN2 or other molecules that interact with ATXN2).

In some embodiments, gene therapies, such as those involving administration of a compounds encoding useful therapeutic agents, useful in the treatment of ALS are provided in US Patent Application Publication No. 20110142789A1, published Jun. 16, 2011, entitled “Compositions and Methods for the Diagnosis and Treatment of Amyotrophic Lateral Sclerosis”; US Patent Application Publication No. 20100047261A1, published Feb. 25, 2010, entitled “Base-modified RNA for increasing the expression of a protein”; US Patent Application Publication No. 20100203076A1, published Aug. 12, 2010, entitled “Complexes of RNA and cationic peptides for transfection and for immunostimulation”; U.S. Pat. No. 10,815,463B2, published Oct. 27, 2020, entitled “Messenger UNA molecules and uses thereof”; U.S. Pat. No. 11,155,817B2, published Oct. 26, 2021, entitled “Therapeutic for treatment of diseases including the central nervous system”; US Patent Application Publication No. 20180327471A1, published Nov. 15, 2018, entitled “Translatable molecules and synthesis thereof”; the entire contents of each of which are herein incorporated by reference.

Certain gene therapies provided in this section may be useful in treating ALS by modulating the activity of genes and gene products other than ATXN2 genes/gene products, such as C9orf72, SOD1, and/or FUS genes/gene products.

### Molecular Payloads Targeting C9orf72

The C9orf72 gene, which encodes the chromosome 9 open reading frame 72 protein, and mutations therein, are implicated in ALS, which predominantly affects upper and lower motor neurons. Modulation of C9orf72 expression and activity (e.g., by suppressing the expression of mutant C9orf72 and/or activity of the protein encoded thereby) therefore in some embodiments can have a therapeutic effect in subjects with ALS.

### Oligonucleotides

C9orf72 expression and/or activity in some embodiments can be modulated by the use of oligonucleotides targeting C9orf72 sequences.

In some embodiments, an oligonucleotide useful for the treatment of ALS, e.g., targeting (e.g., directly or indirectly modulating the expression or activity of) C9orf72, comprises a region of complementarity to a C9orf72 transcript provided in Table 3, e.g., provided by any one of SEQ ID NOs: 393-395.

In some embodiments, examples of oligonucleotides useful for the treatment of ALS, e.g., targeting (e.g., directly or indirectly modulating the expression or activity of) C9orf72, are provided in U.S. Pat. No. 10,577,604B2, published Mar. 3, 2020, entitled “Methods for monitoring C9ORF72 expression”; U.S. Pat. No. 10,443,052B2, published Oct. 15, 2019, entitled “Compositions for modulating C9ORF72 expression”; U.S. Pat. No. 10,793,855B2, published Oct. 6, 2020, entitled “Compositions for modulating expression of C9ORF72 antisense transcript”; U.S. Pat. No. 10,815,483B2, published Oct. 27, 2020, entitled “Compositions for modulating C9ORF72 expression”; U.S. Pat. No. 11,260,073B2, published Mar. 1, 2022, entitled “Compositions and methods for modulating C9ORF72”; U.S. Pat. No. 10,407,678B2, published Oct. 9, 2019, entitled “Compositions for modulating expression of C9ORF72 antisense transcript”; U.S. Pat. No. 11,162,096B2, published Nov. 2, 2021, entitled “Compositions for modulating expression of C9ORF72 antisense transcript”; U.S. Pat. No. 10,066,228B2, published Sep. 4, 2018, entitled “Oligonucleotides for treating expanded repeat diseases”; U.S. Pat. No. 11,345,915B2, published May 31, 2022, entitled “RNA modulating oligonucleotides with improved characteristics for the treatment of neuromuscular disorders”; U.S. Pat. No. 9,963,699B2, published May 8, 2018, entitled “Methods for modulating C9ORF72 expression”; U.S. Pat. No. 10,221,414B2, published Mar. 5, 2019, entitled “Compositions for modulating C9ORF72 expression”; US Patent Application Publication No. 20160108396A1, published Apr. 21, 2016, entitled “Oligomers targeting hexanucleotide repeat expansion in human C9ORF72 gene”; U.S. Pat. No. 10,538,762, published Jan. 21, 2020, entitled “Allele selective inhibition of mutant C9orf72 foci expression by duplex RNAS targeting the expanded hexanucleotide repeat”; U.S. Pat. No. 10,597,660B2, published Mar. 24, 2020, entitled “Compositions and methods of treating amyotrophic lateral sclerosis (ALS)”; U.S. Pat. No. 11,118,179B2, published Sep. 14, 2021, entitled “Mixed tricyclo-DNA, 2′-modified RNA oligonucleotide compositions and uses thereof”; US Patent Application Publication No. 20210284629A1, published Sep. 16, 2021, entitled “Methods and compounds for the treatment of genetic disease”; US Patent Application Publication No. 20210269825A1, published Sep. 2, 2021, entitled “Compositions and methods for reducing spliceopathy and treating rna dominance disorders”; US Patent Application Publication No. 20200385737A1, published Dec. 10, 2020, entitled “OLIGONUCLEOTIDE-BASED MODULATION OF C9orf72”; US Patent Application Publication No. 20200385723A1, published Dec. 10, 2020, entitled “Anti-c9orf72 oligonucleotides and related methods”; US Patent Application Publication No. 20220145300A1, published May 12, 2022, entitled “Oligonucleotide compositions and methods of use thereof”; US Patent Application Publication No. 20210032620A1, published Feb. 4, 2021, entitled “Oligonucleotide compositions and methods thereof”; International PCT Application Publication No. WO2021119226A1, published Dec. 10, 2020, entitled “Human chromosome 9 open reading frame 72 (c9orf72) irna agent compositions and methods of use thereof”; US Patent Application Publication No. 20210340535A1, published Nov. 4, 2021, entitled “DUAL-ACTING siRNA BASED MODULATION OF C9orf72”; International PCT Application Publication No. WO2021205005A2, published Oct. 14, 2021, entitled “Antisense sequences for treating amyotrophic lateral sclerosis”; the entire contents of each of which are herein incorporated by reference.

Certain oligonucleotides provided in this section may be useful in treating ALS by modulating the activity of genes and/or gene products other than C9orf72 genes/gene products, such as ATXN2, SOD1, and/or FUS genes/gene products.

### Polypeptides

C9orf72 expression and/or activity of the protein encoded thereby in some embodiments can be modulated by the use of polypeptides, such as polypeptides that can interact with C9orf72 and/or its encoded protein (e.g., to modulate its biological activity and/or its interaction with other biomolecules).

In some embodiments, examples of polypeptides (e.g., peptides; proteins, such as enzymes; antibodies; etc.) useful in the treatment of ALS are provided in U.S. Pat. No. 10,295,547B2, published May 21, 2019, entitled “Use and treatment of di-amino acid repeat-containing proteins associated with ALS”; U.S. Pat. No. 11,197,911B2, published Dec. 14, 2021, entitled “Peptidylic inhibitors targeting C9ORF72 hexanucleotide repeat-mediated neurodegeneration”; US Patent Application Publication No. 20220153874A1, published May 19, 2022, entitled “Human-derived anti-(poly-ga) dipeptide repeat (dpr) antibody”; U.S. Pat. No. 9,329,182B2, published May 3, 2016, entitled “Method of treating motor neuron disease with an antibody that agonizes MuSK”; the entire contents of each of which are herein incorporated by reference.

Certain polypeptides provided in this section may be useful in treating ALS by modulating the activity of genes and gene products other than C9orf72 genes/gene products, such as ATXN2, SOD1, and/or FUS genes/gene products.

### Small Molecules

C9orf72 expression and/or the activity of the protein it encodes in some embodiments can be modulated by the use of small molecules that can modulate C9orf72 (e.g., to modulate its biological activity, its expression, and/or its interaction with other biomolecules).

In some embodiments, examples of small molecules useful in the treatment of ALS are provided in U.S. Pat. No. 10,675,293B2, published Jun. 9, 2020, entitled “Nucleoside agents for the reduction of the deleterious activity of extended nucleotide repeat containing genes”; International PCT Application Publication No. WO2021174167A1, published Sep. 2, 2021, entitled “Compounds and methods for modulating splicing”; U.S. Pat. No. 11,241,417B2, published Feb. 8, 2022, entitled “Compositions and methods for the treatment and prevention of neurological disorders”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating ALS by modulating the activity of genes and gene products other than C9orf72 genes/gene products, such as ATXN2, SOD1, and/or FUS genes/gene products.

### Gene Therapies

C9orf72 expression and/or activity in some embodiments can be modulated by the use of gene therapies that can modulate C9orf72 (e.g., by delivery of nucleic acids encoding C9orf72 or other molecules that interact with the protein it encodes).

In some embodiments, gene therapies, such as those involving administration of a compounds encoding useful therapeutic agents, useful in the treatment of ALS are provided in U.S. Pat. No. 10,597,660B2, published Mar. 24, 2020, entitled “Compositions and methods of treating amyotrophic lateral sclerosis (ALS)”; US Patent Application Publication No. 20210269825A1, published Sep. 2, 2021, entitled “Compositions and methods for reducing spliceopathy and treating ma dominance disorders”; U.S. Pat. No. 10,801,027B2, published Oct. 13, 2020, entitled “Inhibitors of SRSF1 to treat neurodegenerative disorders”; International PCT Application Publication No. WO2021160464A1, published Aug. 19, 2021, entitled “Gene therapy”; the entire contents of each of which are herein incorporated by reference.

Certain gene therapies provided in this section may be useful in treating ALS by modulating the activity of genes and gene products other than C9orf72 genes/gene products, such as ATXN2, SOD1, and/or FUS genes/gene products.

### Molecular Payloads Targeting FUS

The FUS gene, which encodes RNA-binding protein FUS/TLS, and mutations therein, are implicated in ALS, which predominantly affects upper and lower motor neurons. Modulation of FUS expression and activity (e.g., by suppressing the expression of mutant FUS and/or activity of the protein encoded thereby) therefore in some embodiments can have a therapeutic effect in subjects with ALS.

### Oligonucleotides

FUS expression and/or activity in some embodiments can be modulated by the use of oligonucleotides targeting FUS sequences.

In some embodiments, an oligonucleotide useful for the treatment of ALS, e.g., targeting (e.g., directly or indirectly modulating the expression or activity of) FUS, comprises a region of complementarity to a FUS transcript provided in Table 3, e.g., provided by any one of SEQ ID NOs: 401-404.

In some embodiments, examples of oligonucleotides useful for the treatment of ALS, e.g., targeting (e.g., directly or indirectly modulating the expression or activity of) FUS, are provided in International PCT Application Publication No. WO2020243292A1, published Dec. 3, 2020, entitled “Compounds and methods for reducing fus expression”; U.S. Pat. No. 11,332,733B2, published May 17, 2022, entitled “Modified compounds and uses thereof”; US Patent Application Publication No. 20100256223A1, published Oct. 7, 2010, entitled “Oligonucleotides for modulating target ma activity”; U.S. Pat. No. 9,150,860B2, published Oct. 6, 2015, entitled “FUS/TLS-based compounds and methods for diagnosis, treatment and prevention of amyotrophic lateral sclerosis and related motor neuron diseases”; US Patent Application Publication No. 20120252875A1, published Oct. 4, 2012, entitled “Methods and compositions for treating diseases, disorders or injury of the CNS”; U.S. Pat. No. 10,781,445B2, published Sep. 22, 2020, entitled “Decoy oligonucleotides for the treatment of diseases”; US Patent Application Publication No. 20190127733A1, published May 2, 2019, entitled “Oligonucleotide compositions and methods thereof”; U.S. Pat. No. 11,197,883B2, published Dec. 14, 2021, entitled “Inhibition of stress granule formation through manipulation of UBAP2L”; US Patent Application Publication No. 20210169914A1, published Jun. 10, 2021, entitled “Nucleic acids and nucleic acid analogs for treating, preventing, and disrupting pathological polynucleotide-binding protein inclusions”; International PCT Application Publication No. WO2021203043A2, published Oct. 7, 2021, entitled “Targeted inhibition using engineered oligonucleotides”; International PCT Application Publication No. WO2021207854A1, published Oct. 21, 2021, entitled “Compositions and methods for inhibiting tdp-43 and fus aggregation”; the entire contents of each of which are herein incorporated by reference.

Certain oligonucleotides provided in this section may be useful in treating ALS by modulating the activity of genes and/or gene products other than FUS genes/gene products, such as ATXN2, SOD1, and/or C9orf72 genes/gene products.

### Polypeptides

FUS expression and/or activity of the protein encoded thereby in some embodiments can be modulated by the use of polypeptides, such as polypeptides that can interact with FUS nucleic acids and/or its encoded protein (e.g., to modulate its biological activity, its localization within the cell, and/or its interaction with other biomolecules).

In some embodiments, examples of polypeptides (e.g., peptides; proteins, such as enzymes; antibodies; etc.) useful in the treatment of ALS are provided in U.S. Pat. No. 1,132,504B2, published May 17, 2022, entitled “Methods of reducing FUS/TLS- or TDP-43-mediated neuronal cytotoxicity by UPF1”; US Patent Application Publication No. 20180360925A1, published Dec. 20, 2018, entitled “Extracellular dna as a therapeutic target in neurodegeneration”; the entire contents of each of which are herein incorporated by reference.

Certain polypeptides provided in this section may be useful in treating ALS by modulating the activity of genes and gene products other than FUS genes/gene products, such as ATXN2, SOD1, and/or C9orf72 genes/gene products.

### Small Molecules

FUS expression and/or the activity of the protein it encodes in some embodiments can be modulated by the use of small molecules that can modulate FUS (e.g., to modulate its biological activity, its expression, its localization, and/or its interaction with other biomolecules).

In some embodiments, examples of small molecules useful in the treatment of ALS are provided in US Patent Application Publication No. 20120272345A1, published Oct. 25, 2012, entitled “Diagnosis marker, diagnosis method and therapeutic agent for amyotrophic lateral sclerosis, and animal model and cell model developing amyotrophic lateral sclerosis”; U.S. Pat. No. 10,159,670B2, published Dec. 25, 2018, entitled “Methods of diagnosing and treating motor neuron diseases and other cellular stress-related diseases”; US Patent Application Publication No. 20200216563A1, published Jul. 9, 2020, entitled “Hdac6 and protein aggregation”; International PCT Application Publication No. WO2021174167A1, published Sep. 2, 2021, entitled “Compounds and methods for modulating splicing”; US Patent Application Publication No. 20200368267A1, published Nov. 26, 2020, entitled “Prophylactic and/or therapeutic agent for amyotrophic lateral sclerosis”; US Patent Application Publication No. 20220071955A1, published Mar. 10, 2022, entitled “Methods of Treatment, Prevention and Diagnosis”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating ALS by modulating the activity of genes and gene products other than FUS genes/gene products, such as ATXN2, SOD1, and/or C9orf72 genes/gene products.

### Gene Therapies

FUS expression and/or activity in some embodiments can be modulated by the use of gene therapies that can modulate FUS (e.g., by delivery of nucleic acids encoding FUS or other molecules that interact with FUS transcripts or the protein encoded by FUS).

In some embodiments, gene therapies, such as those involving administration of a compounds encoding useful therapeutic agents, useful in the treatment of ALS are provided in International PCT Application Publication No. WO2021205010A1, published Oct. 14, 2021, entitled “Nucleic acids encoding human FUS protein and use in the treatment of amyotrophic lateral sclerosis (ALS)”; the entire contents of each of which are herein incorporated by reference.

Certain gene therapies provided in this section may be useful in treating ALS by modulating the activity of genes and gene products other than FUS genes/gene products, such as ATXN2, SOD1, and/or C9orf72 genes/gene products.

### Molecular Payloads Targeting PIKFYVE

The PIKFYVE gene, which encodes the phosphatidylinositol-3-phosphate 5-kinase type III (PIPKIII) protein, and mutations therein, are implicated in ALS. Modulation of PIKFYVE expression and activity (e.g., by suppressing the expression and/or activity of mutant PIPKIII protein and/or its interactions with other proteins) therefore in some embodiments can have a therapeutic effect in subjects with ALS.

### Oligonucleotides

PIKFYVE (and/or PIPKIII protein encoded by PIKFYVE) expression and/or activity in some embodiments can be modulated by the use of oligonucleotides targeting PIKFYVE sequences.

In some embodiments, an oligonucleotide useful for the treatment of ALS, e.g., targeting (e.g., directly or indirectly modulating the expression or activity of) PIKFYVE, comprises a region of complementarity to a PIKFYVE transcript provided in Table 3, e.g., provided by any one of SEQ ID NOs: 143-148.

In some embodiments, examples of oligonucleotides useful for the treatment of ALS, e.g., targeting (e.g., directly or indirectly modulating the expression or activity of) PIKFYVE, are provided in US20220411804A1, published Dec. 29, 2022, entitled “Pikfyve antisense oligonucleotides”; the entire contents of which are herein incorporated by reference.

Certain oligonucleotides provided in this section may be useful in treating ALS by modulating the activity of genes and/or gene products other than PIKFYVE genes/gene products, such as other genes/gene products associated with ALS.

### Polypeptides

PIKFYVE expression and/or activity in some embodiments can be modulated by the use of PIPKIII polypeptides or polypeptides that can interact with PIPKIII (e.g., to modulate its biological activity and/or its interaction with other biomolecules).

In some embodiments, examples of polypeptides (e.g., peptides; proteins, such as enzymes; antibodies; etc.) useful in the treatment of ALS include PIPKIII protein and functional fragments thereof.

Certain polypeptides provided in this section may be useful in treating ALS by modulating the activity of genes and gene products other than PIKFYVE genes/gene products, such as other genes/gene products associated with ALS.

### Small Molecules

PIKFYVE expression and/or activity in some embodiments can be modulated by the use of small molecules that can modulate PIPKIII (e.g., to modulate its biological activity, its expression, and/or its interaction with other biomolecules).

In some embodiments, examples of small molecules useful in the treatment of ALS are provided in US20190192527A1, published Jun. 27, 2019, entitled “Compositions comprising pikfyve inhibitors and methods related to inhibition of rank signaling”; WO2017040971A1, published Mar. 9, 2017, entitled “Methods of using inhibitors of pikfyve for the treatment of lysosomal storage disorders and neurodegenerative diseases”; WO2022086993A1, published Apr. 28, 2022, entitled “Novel inhibitors of pikfyve and methods using same”; US20210139505A1, published May 13, 2021, entitled “PIKfyve Inhibitors”; U.S. Ser. No. 10/758,545B2, published Sep. 1, 2020, entitled “Methods to treat neurological diseases”; U.S. Ser. No. 11/066,410B2, published Jul. 20, 2021, entitled “Fused triazolo-pyrimidine compounds having useful pharmaceutical application”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating ALS by modulating the activity of genes and gene products other than PIKFYVE genes/gene products, such as other genes/gene products associated with ALS.

### Gene Therapies

PIKFYVE expression and/or activity in some embodiments can be modulated by the use of gene therapies that can modulate PIKFYVE (e.g., by delivery of nucleic acids encoding PIKFYVE or other molecules that interact with PIKFYVE).

In some embodiments, gene therapies, such as those involving administration of a compounds encoding useful therapeutic agents, useful in the treatment of ALS include payloads which encode PIPKIII or functional fragments thereof.

Certain gene therapies provided in this section may be useful in treating ALS by modulating the activity of genes and gene products other than PIKFYVE genes/gene products, such as other genes/gene products associated with ALS.

### Molecular Payloads Targeting SYF2

The SYF2 gene, which encodes the pre-mRNA-splicing factor SYF2 protein, and mutations therein, are implicated in ALS. Modulation of SYF2 expression and activity (e.g., by suppressing the expression and/or activity of mutant pre-mRNA-splicing factor SYF2 protein and/or its interactions with other proteins) therefore in some embodiments can have a therapeutic effect in subjects with ALS.

### Oligonucleotides

SYF2 (and/or pre-mRNA-splicing factor SYF2 protein encoded by SYF2) expression and/or activity in some embodiments can be modulated by the use of oligonucleotides targeting SYF2 sequences.

In some embodiments, an oligonucleotide useful for the treatment of ALS, e.g., targeting (e.g., directly or indirectly modulating the expression or activity of) SYF2, comprises a region of complementarity to a SYF2 transcript provided in Table 3, e.g., provided by any one of SEQ ID NOs: 167-168.

In some embodiments, examples of oligonucleotides useful for the treatment of ALS, e.g., targeting (e.g., directly or indirectly modulating the expression or activity of) SYF2, are provided in US20230066380A1, published Mar. 2, 2023, entitled “Antagonism as a therapy for tdp-43 proteinopathies”; the entire contents of which are herein incorporated by reference.

Certain oligonucleotides provided in this section may be useful in treating ALS by modulating the activity of genes and/or gene products other than SYF2 genes/gene products, such as other genes/gene products associated with ALS.

### Polypeptides

SYF2 expression and/or activity in some embodiments can be modulated by the use of pre-mRNA-splicing factor SYF2 polypeptides or polypeptides that can interact with pre-mRNA-splicing factor SYF2 (e.g., to modulate its biological activity and/or its interaction with other biomolecules).

In some embodiments, examples of polypeptides (e.g., peptides; proteins, such as enzymes; antibodies; etc.) useful in the treatment of ALS include pre-mRNA-splicing factor SYF2 protein and functional fragments thereof.

Certain polypeptides provided in this section may be useful in treating ALS by modulating the activity of genes and gene products other than SYF2 genes/gene products, such as other genes/gene products associated with ALS.

### Small Molecules

SYF2 expression and/or activity in some embodiments can be modulated by the use of small molecules that can modulate pre-mRNA-splicing factor SYF2 protein (e.g., to modulate its biological activity, its expression, and/or its interaction with other biomolecules).

In some embodiments, examples of small molecules useful in the treatment of ALS are small molecules that increase or decrease expression of SYF2, and/or that increase or decrease pre-mRNA-splicing factor SYF2 protein levels or activity.

Certain small molecules provided in this section may be useful in treating ALS by modulating the activity of genes and gene products other than SYF2 genes/gene products, such as other genes/gene products associated with ALS.

### Gene Therapies

SYF2 expression and/or activity in some embodiments can be modulated by the use of gene therapies that can modulate SYF2 (e.g., by delivery of nucleic acids encoding SYF2 or other molecules that interact with SYF2).

In some embodiments, gene therapies, such as those involving administration of a compounds encoding useful therapeutic agents, useful in the treatment of ALS include payloads which encode pre-mRNA-splicing factor SYF2 protein or functional fragments thereof.

Certain gene therapies provided in this section may be useful in treating ALS by modulating the activity of genes and gene products other than SYF2 genes/gene products, such as other genes/gene products associated with ALS.

### Molecular Payloads Targeting UNC13A

The UNC13A gene, which encodes the unc-13 homolog A protein, and mutations therein, are implicated in ALS. Modulation of UCN13A expression and activity (e.g., by suppressing the expression and/or activity of mutant unc-13 homolog A protein and/or its interactions with other proteins) therefore in some embodiments can have a therapeutic effect in subjects with ALS.

### Oligonucleotides

UNC13A (and/or unc-13 homolog A protein encoded by UNC13A) expression and/or activity in some embodiments can be modulated by the use of oligonucleotides targeting UNC13A sequences.

In some embodiments, an oligonucleotide useful for the treatment of ALS, e.g., targeting (e.g., directly or indirectly modulating the expression or activity of) UNC13A, comprises a region of complementarity to a UNC13A transcript provided in Table 3, e.g., provided by any one of SEQ ID NOs: 169 and 810-818.

In some embodiments, examples of oligonucleotides useful for the treatment of ALS, e.g., targeting (e.g., directly or indirectly modulating the expression or activity of) UNC13A, are provided in WO2022246251A2, published Nov. 24, 2022, entitled “Compounds for modulating unc13a expression”; WO2023102225, published Dec. 2, 2022, entitled “Treatment of neurological diseases using modulators of unc13a gene transcripts”; US20230125137, published Apr. 27, 2023, entitled “Unc13a antisense oligonucleotides”; WO2022122872, published Jun. 16, 2022, entitled “Therapeutics for the treatment of neurodegenerative disorders”; WO2023102242, published Jun. 8, 2023, entitled “Splice switcher antisense oligonucleotides with modified backbone chemistries”; WO2023104964, published Jun. 15, 2023, entitled “Therapeutics for the treatment of neurodegenerative disorders”; and US20220033818A1, published Feb. 3, 2023, entitled “Oligonucleotides targeting ma binding protein sites”; the entire contents of which are herein incorporated by reference.

Certain oligonucleotides provided in this section may be useful in treating ALS by modulating the activity of genes and/or gene products other than UNC13A genes/gene products, such as other genes/gene products associated with ALS.

### Polypeptides

UNC13A expression and/or activity in some embodiments can be modulated by the use of unc-13 homolog A polypeptides or polypeptides that can interact with unc-13 homolog A (e.g., to modulate its biological activity and/or its interaction with other biomolecules).

In some embodiments, examples of polypeptides (e.g., peptides; proteins, such as enzymes; antibodies; etc.) useful in the treatment of ALS include unc-13 homolog A protein and functional fragments thereof.

Certain polypeptides provided in this section may be useful in treating ALS by modulating the activity of genes and gene products other than UNC13A genes/gene products, such as other genes/gene products associated with ALS.

### Small Molecules

UNC13A expression and/or activity in some embodiments can be modulated by the use of small molecules that can modulate unc-13 homolog A protein (e.g., to modulate its biological activity, its expression, and/or its interaction with other biomolecules).

In some embodiments, examples of small molecules useful in the treatment of ALS are small molecules that increase or decrease expression of UNC13A, and/or that increase or decrease unc-13 homolog A protein levels or activity.

Certain small molecules provided in this section may be useful in treating ALS by modulating the activity of genes and gene products other than UNC13A genes/gene products, such as other genes/gene products associated with ALS.

### Gene Therapies

UNC13A expression and/or activity in some embodiments can be modulated by the use of gene therapies that can modulate UNC13A (e.g., by delivery of nucleic acids encoding UNC13A or other molecules that interact with UNC13A).

In some embodiments, gene therapies, such as those involving administration of a compounds encoding useful therapeutic agents, useful in the treatment of ALS include payloads which encode unc-13 homolog A protein or functional fragments thereof.

Certain gene therapies provided in this section may be useful in treating ALS by modulating the activity of genes and gene products other than UNC13A genes/gene products, such as other genes/gene products associated with ALS.

### Molecular Payloads for the Treatment of Spinocerebellar Ataxia

Various molecular payloads may be useful in the treatment of spinocerebellar ataxia (e.g., SCA1, SCA2, SCA3, and/or other types of SCA), including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of spinocerebellar ataxia may include, in some embodiments, molecular payloads which modulate (e.g., increase or decrease) expression or activity of ATXN1, ATXN2, ATXN3, and/or MSH3.

Examples of oligonucleotides useful for the treatment of spinocerebellar ataxia, e.g., oligonucleotides targeting (e.g., directly or indirectly modulating the expression or activity of) genes associated with cerebellar ataxia (e.g., ATXN1, ATXN2, ATXN3, MSH3, etc.), include those listed in Table 7 below. Each oligonucleotide provided in Table 7 may have any modification pattern disclosed herein.

TABLE 7
Oligonucleotides for the treatment of spinocerebellar ataxia

| | | Oligo- | |
|---|---|---|---|
| SEQ | | nucleotide | |
| ID | Target | Sequence | |
| NO: | gene | (5′-3′) | Oligonucleotide Structure (5′-3′) |
| 996 | ATXN1 | CCCGTATTCC | mes meo meo eo es ds ds ds mds mds ds mds ds ds |
| | | TCTTACCATC | ds meo meo es es me, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 997 | ATXN1 | GCACGGTATT | es meo eo meo es ds ds ds ds ds ds ds ds ds ds |
| | | AGTGTCTTCA | meo eo es mes e, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 998 | ATXN1 | GCTTCTCAAA | es meo eo eo mes ds mds ds ds ds ds mds ds ds ds |
| | | TCAGGTGTAC | eo eo es es me, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 999 | ATXN1 | GCCTTTATAA | es meo meo eo es ds ds ds ds ds mds ds ds ds ds |
| | | CTTTTCTTTC | meo eo es es me, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 1000 | ATXN1 | TTCAGTTTAG | es eo meo eo es ds ds ds ds ds ds ds ds mds ds eo |
| | | TTGCAGCCAT | meo mes es e, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 1001 | ATXN1 | TCAGTTTAGT | es mes es eo es ds ds ds ds ds ds ds mds eo es |
| | | TGCAGCC | mes me, wherein: m = 5-methyl modified nucleobase, |
| | | | e = 2′-MOE sugar moiety, d = 2′-β-D deoxyribosyl |
| | | | sugar moiety, s = phosphorothioate internucleoside |
| | | | linkage, o = phosphodiester internucleoside linkage |
| 30 | ATXN2 | TGGATTCTGT | es eo eo eo eo eo mds ds ds ds ds mds ds ds ds ds |
| | | ACTTTTCTCA | meo es mes e, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 31 | ATXN2 | GTACTTTTCT | es eo eo meo eo ds ds ds mds ds mds ds ds ds ds |
| | | CATGTGCGGC | eo meo es es me, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 32 | ATXN2 | CTTCACATTT | es eo eo eo eo ds ds ds ds ds ds ds ds ds ds eo |
| | | CGATCCAACA | eo es es e, wherein: e = 2′-MOE sugar moiety, d = 2′- |
| | | | β-D deoxyribosyl sugar moiety, s = phosphorothioate |
| | | | internucleoside linkage, o = phosphodiester |
| | | | internucleoside linkage |
| 33 | ATXN2 | GCTAACTGGT | es es es es es ds ds ds ds ds ds ds ds ds ds es |
| | | TTGCCCTTGC | es es es e, wherein: e = 2′-MOE sugar moiety, d = 2′- |
| | | | β-D deoxyribosyl sugar moiety, s = phosphorothioate |
| | | | internucleoside linkage, o = phosphodiester |
| | | | internucleoside linkage |
| 34 | ATXN2 | CCCAAAGGGT | es es es es es ds ds ds ds ds ds ds ds ds ds es |
| | | TAATTAGGAT | es es es e, wherein: e = 2′-MOE sugar moiety, d = 2′- |
| | | | β-D deoxyribosyl sugar moiety, s = phosphorothioate |
| | | | internucleoside linkage, o = phosphodiester |
| | | | internucleoside linkage |
| 35 | ATXN2 | CTCTCCATTA | es es es es es ds ds ds ds ds ds ds ds ds ds es |
| | | TTTCTTCACG | es es es e, wherein: e = 2′-MOE sugar moiety, d = 2′- |
| | | | β-D deoxyribosyl sugar moiety, s = phosphorothioate |
| | | | internucleoside linkage, o = phosphodiester |
| | | | internucleoside linkage |
| 36 | ATXN2 | CTGGTACAGT | es es es es es ds ds ds ds ds ds ds ds ds ds es |
| | | TGCTGCTGCT | es es es e, wherein: e = 2′-MOE sugar moiety, d = 2′- |
| | | | β-D deoxyribosyl sugar moiety, s = phosphorothioate |
| | | | internucleoside linkage, o = phosphodiester |
| | | | internucleoside linkage |
| 37 | ATXN2 | GGAGCTGGAG | es es es es es ds ds ds ds ds ds ds ds ds ds es |
| | | AACCATGAGC | es es es e, wherein: e = 2′-MOE sugar moiety, d = 2′- |
| | | | β-D deoxyribosyl sugar moiety, s = phosphorothioate |
| | | | internucleoside linkage, o = phosphodiester |
| | | | internucleoside linkage |
| 38 | ATXN2 | TGCATAGATT | es es es es es ds ds ds ds ds ds ds ds ds ds es |
| | | CCATCAAAAG | es es es e, wherein: e = 2′-MOE sugar moiety, d = 2′- |
| | | | β-D deoxyribosyl sugar moiety, s = phosphorothioate |
| | | | internucleoside linkage, o = phosphodiester |
| | | | internucleoside linkage |
| 39 | ATXN2 | GTGGGATACA | es es es es es ds ds ds ds sd ds ds ds ds ds es |
| | | AATTCTAGGC | es es es e, wherein: e = 2′-MOE sugar moiety, d = 2′- |
| | | | β-D deoxyribosyl sugar moiety, s = phosphorothioate |
| | | | internucleoside linkage, o = phosphodiester |
| | | | internucleoside linkage |
| 40 | ATXN2 | CCCATACGCG | es es es es es ds ds ds ds ds ds ds ds ds ds es |
| | | GTGAATTCTG | es es es e, wherein: e = 2′-MOE sugar moiety, d = 2′- |
| | | | β-D deoxyribosyl sugar moiety, s = phosphorothioate |
| | | | internucleoside linkage, o = phosphodiester |
| | | | internucleoside linkage |
| 41 | ATXN2 | CTGCTAACTG | mes eo eo meo ds ds ds mds ds ds ds ds ds ds eo |
| | | GTTTGCCCTT | meo meo mes es e, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 42 | ATXN2 | TGTACTTCAC | es eo eo eo meo eo ds mds ds mds ds ds ds ds ds |
| | | ATTTGGAGCC | ds eo es mes me, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 43 | ATXN2 | CCTATCATCA | mes meo eo eo eo meo ds ds mds ds ds ds ds ds mds |
| | | TTTTCCAGGG | mds eo es es e, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 44 | ATXN2 | TCTGTACTTT | es meo eo eo es ds mds ds ds ds ds mds ds mds ds |
| | | TCTCATGTGC | eo eo es es me, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 1002 | ATXN3 | GCTCATTTAT | es meo eo meo es ds ds ds ds ds ds mds ds mds ds |
| | | TCTCAAGTAC | eo eo es es me, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 1003 | ATXN3 | GCACCATATA | es meo eo meo mes ds ds ds ds ds ds ds ds mds ds |
| | | TATCTCAGAA | meo eo es es e, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 1004 | ATXN3 | GTTAATACTT | es eo eo eo es ds ds mds ds ds ds ds ds mds mds |
| | | TTTCCAGCCT | eo eo mes mes e, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 1005 | ATXN3 | AGCCAATATT | es eo meo meo es ds ds ds ds ds ds ds ds ds ds eo |
| | | TATAGGTGCT | eo es mes e, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 1006 | ATXN3 | GCCATTAATC | es meo meo eo eo eo ds ds ds mds ds ds ds ds mds |
| | | TATACTGAAT | ds eo es es e, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 1007 | ATXN3 | GCATATTGGT | es meo eo eo eo eo ds ds ds ds ds ds ds mds ds |
| | | TTTCTCATTT | mds eo es es e, wherein: m = 5-methyl modified |
| | | | nucleobase, e = 2′-MOE sugar moiety, d = 2′-β-D |
| | | | deoxyribosyl sugar moiety, s = phosphorothioate |
| | | | internucleoside linkage, o = phosphodiester |
| | | | internucleoside linkage |
| 1008 | ATXN3 | GCTGTTGCTG | All 2′-MOE sugar moiety; all internucleoside |
| | | CTTTTGCTGC | linkages phosphorothioate; each C is 5-methylC |
| | | TG | |
| 1009 | ATXN3 | CTGTTGCTGC | All 2′-MOE sugar moiety; all internucleoside |
| | | TTTTGCTGCT | linkages phosphorothioate; each C is 5-methylC |
| 1010 | ATXN3 | GAGATATGTT | All 2′-MOE sugar moiety; all internucleoside |
| | | TCTGGAACTA | linkages phosphorothioate; each C is 5-methylC |
| | | CC | |
| 1011 | ATXN3 | GCTTCTCGTC | All 2′-MOE sugar moiety; all internucleoside |
| | | TCTTCCGAAG | linkages phosphorothioate; each C is 5-methylC |
| | | C | |
| 1012 | ATXN3 | CCGAAGCTCT | All 2′-MOE sugar moiety; all internucleoside |
| | | TCTGAAGTAA | linkages phosphorothioate; each C is 5-methylC |
| 1013 | ATXN3 | GAACTCTGTC | All 2′-MOE sugar moiety; all internucleoside |
| | | CTGATAGGTC | linkages phosphorothioate; each C is 5-methylC |
| 1014 | ATXN3 | CTAGATCACT | All 2′-MOE sugar moiety; all internucleoside |
| | | CCCAAGTGCT | linkages phosphorothioate; each C is 5-methylC |
| 1015 | ATXN3 | ATAGGTCCCG | All 2′-MOE sugar moiety; all internucleoside |
| | | CTGCTGCT | linkages phosphorothioate; each C is 5-methylC |

In each sequence listed in Table 7, each T may be optionally and independently replaced with a U, and each U may be optionally and independently replaced with a T.

Examples of small molecules useful for the treatment of spinocerebellar ataxia (e.g., SCA1, SCA2, SCA3, and/or other types of SCA) include: baclofen, chlorzoxazone, A71623, and pharmaceutically acceptable salts, co-crystals, tautomers, stereoisomers, solvates, hydrates, polymorphs, isotopically enriched compounds, and prodrugs thereof.

Examples of polypeptides useful for the treatment of spinocerebellar ataxia (e.g., SCA1, SCA2, SCA3, and/or other types of SCA) include antibodies, proteins, peptides, and enzymes. In some embodiments, polypeptides useful for the treatment of SCA include: A71623 (Boc-Trp-Lys(Tac)-Asp-N-methyl-Phe-NH₂) (SEQ ID NO: 807).

### Molecular Payloads Targeting ATXN1

The ATXN1 gene, which encodes the ataxin-1 protein, and mutations therein, are implicated in SCA type 1 (SCA1). Modulation of ATXN1 expression and activity (e.g., by suppressing the expression and/or activity of mutant ATXN1 protein and/or its interactions with other proteins) therefore in some embodiments can have a therapeutic effect in subjects with SCA1.

### Oligonucleotides

ATXN1 (and/or ataxin-1 protein encoded by ATXN1) expression and/or activity in some embodiments can be modulated by the use of oligonucleotides targeting ATXN1 sequences.

In some embodiments, an oligonucleotide useful for the treatment of SCA, e.g., targeting (e.g., directly or indirectly modulating the expression or activity of) ATXN1, comprises a region of complementarity to a ATXN1 transcript provided in Table 3, e.g., provided by any one of SEQ ID NOs: 819-820.

In some embodiments, examples of oligonucleotides useful for the treatment of SCA1, e.g., targeting (e.g., directly or indirectly modulating the expression or activity of) ATXN1, are provided in U.S. Ser. No. 11/542,504B2, published Jan. 3, 2023, entitled “Compounds and methods for modulating ATXN1”; U.S. Ser. No. 11/345,915B2, published May 31, 2022, entitled “RNA modulating oligonucleotides with improved characteristics for the treatment of neuromuscular disorders”; the entire contents of each of which are herein incorporated by reference.

Certain oligonucleotides provided in this section may be useful in treating SCA1 by modulating the activity of genes and/or gene products other than ATXN1 genes/gene products, such as other genes/gene products associated with SCA and/or SCA1.

### Polypeptides

ATXN1 expression and/or activity in some embodiments can be modulated by the use of ataxin-1 polypeptides or polypeptides that can interact with ataxin-1 (e.g., to modulate its biological activity and/or its interaction with other biomolecules).

In some embodiments, examples of polypeptides (e.g., peptides; proteins, such as enzymes; antibodies; etc.) useful in the treatment of SCA1 are provided in U.S. Ser. No. 10/989,719B2, published Apr. 27, 2021, entitled “Methods for treating spinocerebellar ataxia type I using RPA1”; U.S. Ser. No. 10/973,812B2, published Apr. 13, 2021, entitled “Ataxia therapeutic compositions and methods”; the entire contents of each of which are herein incorporated by reference.

Certain polypeptides provided in this section may be useful in treating SCA1 by modulating the activity of genes and gene products other than ATXN1 genes/gene products, such as other genes/gene products associated with SCA and/or SCA1.

### Small Molecules

ATXN1 expression and/or activity in some embodiments can be modulated by the use of small molecules that can modulate ATXN1 (e.g., to modulate its biological activity, its expression, and/or its interaction with other biomolecules).

In some embodiments, examples of small molecules useful in the treatment of SCA1 are provided in U.S. Ser. No. 11/382,897B2, published Jul. 12, 2022, entitled “Therapeutic combination for treatment of cerebellar ataxia”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating SCA1 by modulating the activity of genes and gene products other than ATXN1 genes/gene products, such as other genes/gene products associated with SCA and/or SCA1.

### Gene Therapies

ATXN1 expression and/or activity in some embodiments can be modulated by the use of gene therapies that can modulate ATXN1 (e.g., by delivery of nucleic acids encoding ATXN1 or other molecules that interact with ATXN1).

In some embodiments, gene therapies, such as those involving administration of a compounds encoding useful therapeutic agents, useful in the treatment of SCA1 are provided in US20110016540A1, published Jan. 20, 2011, entitled “Genome editing of genes associated with trinucleotide repeat expansion disorders in animals”; U.S. Ser. No. 10/989,719B2, published Apr. 27, 2021, entitled “Methods for treating spinocerebellar ataxia type I using RPA1”; U.S. Ser. No. 11/027,024B2, published Jun. 8, 2021, entitled “Methods of delivery of transgenes for treating brain diseases”; US20210238226A1, published Aug. 5, 2021, entitled “Methods and compounds for the treatment of genetic disease”; the entire contents of each of which are herein incorporated by reference.

Certain gene therapies provided in this section may be useful in treating SCA1 by modulating the activity of genes and gene products other than ATXN1 genes/gene products, such as other genes/gene products associated with SCA and/or SCA1.

### Molecular Payloads Targeting ATXN2

The ATXN2 gene, which encodes the ataxin-2 protein, and mutations therein, are implicated in SCA type 2 (SCA2). Modulation of ATXN2 expression and activity (e.g., by suppressing the expression and/or activity of mutant ATXN2 protein and/or its interactions with other proteins) therefore in some embodiments can have a therapeutic effect in subjects with SCA2.

### Oligonucleotides

ATXN2 (and/or ataxin-2 protein encoded by ATXN2) expression and/or activity in some embodiments can be modulated by the use of oligonucleotides targeting ATXN2 sequences.

In some embodiments, an oligonucleotide useful for the treatment of SCA, e.g., targeting (e.g., directly or indirectly modulating the expression or activity of) ATXN2, comprises a region of complementarity to a ATXN2 transcript provided in Table 3, e.g., provided by any one of SEQ ID NOs: 396-400.

In some embodiments, examples of oligonucleotides useful for the treatment of SCA2, e.g., targeting (e.g., directly or indirectly modulating the expression or activity of) ATXN2, are provided in Becker et al. (2017) “Therapeutic reduction of ataxin-2 extends lifespan and reduces pathology in TDP-43 mice” Nature 544:367-371; Scoles et al. (2017) “Antisense oligonucleotide therapy for spinocerebellar ataxia type 2” Nature 544:362-366; US Patent Application Publication No. 20110142789A1, published Jun. 16, 2011, entitled “Compositions and Methods for the Diagnosis and Treatment of Amyotrophic Lateral Sclerosis”; US Patent Application Publication No. 20130225659A1, published Aug. 29, 2013, entitled “Modulation of nuclear-retained RNA”; U.S. Pat. No. 1,107,486B2, published Aug. 3, 2021, entitled “Compounds and methods for reducing ATXN2 expression”; US Patent Application Publication No. 20220064639A1, published Mar. 3, 2022, entitled “Compounds and methods for reducing ATXN2 expression”; U.S. Pat. No. 10,533,178B2, published Jan. 14, 2020, entitled “Methods for modulating Ataxin 2 expression”; U.S. Pat. No. 10,006,027B2, published Jun. 26, 2018, entitled “Methods for modulating Ataxin 2 expression”; U.S. Pat. No. 10,308,934B2, published Jun. 4, 2019, entitled “Compositions for modulating Ataxin 2 expression”; U.S. Pat. No. 11,111,494B2, published Sep. 7, 2021, entitled “Compositions for modulating Ataxin 2 expression”; U.S. Pat. No. 11,345,915B2, published May 31, 2022, entitled “RNA modulating oligonucleotides with improved characteristics for the treatment of neuromuscular disorders”; US Patent Application Publication No. 20140378533A1, published Dec. 25, 2014, entitled “Modulation of RNA by repeat targeting”; US Patent Application Publication No. 20150148404A1, published May 28, 2015, entitled “RNA Modulating Oligonucleotides with Improved Characteristics for the Treatment of Neuromuscular Disorders”; US Patent Application Publication No. 20210169914A1, published Jun. 10, 2021, entitled “Nucleic acids and nucleic acid analogs for treating, preventing, and disrupting pathological polynucleotide-binding protein inclusions”; US Patent Application Publication No. 20160040163A1, published Feb. 11, 2016, entitled “DNAi for the modulation of genes”; U.S. Pat. No. 10,174,328B2, published Jan. 8, 2019, entitled “Compositions and methods for treating amyotrophic lateral sclerosis”; US Patent Application Publication No. 20220162615A1, published May 26, 2022, entitled “Methods for reducing ataxin-2 expression”; U.S. Ser. No. 11/345,915B2, published May 31, 2022, entitled “RNA modulating oligonucleotides with improved characteristics for the treatment of neuromuscular disorders”; the entire contents of each of which are herein incorporated by reference.

Certain oligonucleotides provided in this section may be useful in treating SCA2 by modulating the activity of genes and/or gene products other than ATXN2 genes/gene products, such as other genes/gene products associated with SCA and/or SCA2.

### Polypeptides

ATXN2 expression and/or activity in some embodiments can be modulated by the use of ataxin-2 polypeptides or polypeptides that can interact with ataxin-2 (e.g., to modulate its biological activity and/or its interaction with other biomolecules).

In some embodiments, examples of polypeptides (e.g., peptides; proteins, such as enzymes; antibodies; etc.) useful in the treatment of SCA2 are provided in US Patent Application Publication No. 20110142789A1, published Jun. 16, 2011, entitled “Compositions and Methods for the Diagnosis and Treatment of Amyotrophic Lateral Sclerosis”; U.S. Pat. No. 10,066,007B2, published Sep. 4, 2019, entitled “Dipeptide-repeat proteins as therapeutic target in neurodegenerative diseases with hexanucleotide repeat expansion”; U.S. Pat. No. 11,273,149B2, published Mar. 15, 2022, entitled “Compositions and methods for the treatment of amyotrophic lateral sclerosis, Parkinson's disease, Parkinson's disease with dementia, dementia with Lewy bodies, and multiple system atrophy”; U.S. Pat. No. 8,673,852B2, entitled “Methods of treating neuronal disorders using MNTF peptides and analogs thereof”; International PCT Application Publication No. WO2021222168A2, published Nov. 4, 2021, entitled “Compositions and methods for the treatment of tdp-43 proteinopathies”; U.S. Ser. No. 10/973,812B2, published Apr. 13, 2021, entitled “Ataxia therapeutic compositions and methods”; the entire contents of each of which are herein incorporated by reference.

Certain polypeptides provided in this section may be useful in treating SCA2 by modulating the activity of genes and gene products other than ATXN2 genes/gene products, such as other genes/gene products associated with SCA and/or SCA2.

### Small Molecules

ATXN2 expression and/or activity in some embodiments can be modulated by the use of small molecules that can modulate ATXN2 (e.g., to modulate its biological activity, its expression, and/or its interaction with other biomolecules).

In some embodiments, examples of small molecules useful in the treatment of SCA2 are provided in US Patent Application Publication No. 20110142789A1, published Jun. 16, 2011, entitled “Compositions and Methods for the Diagnosis and Treatment of Amyotrophic Lateral Sclerosis”; US Patent Application Publication No. 20130303562A1, published Nov. 14, 2013, entitled “Chemical and RNAi suppressors of neurotoxicity in Huntington's disease”; US Patent Application Publication No. 20140228333A1, published Mar. 29, 2016, entitled “Methods for inhibiting muscle atrophy”; International PCT Application Publication No. WO2021174167A1, published Sep. 2, 2021, entitled “Compounds and methods for modulating splicing”; International PCT Patent Application Publication No. WO2013043669A1, published Mar. 28, 2013, entitled “Peptoid compositions for the treatment of Alzheimer's disease and polyglutamine expansion disorder”; U.S. Pat. No. 10,159,670B2, published Dec. 25, 2018, entitled “Methods of diagnosing and treating motor neuron diseases and other cellular stress-related diseases”; U.S. Pat. No. 9,790,188B2, published Oct. 17, 2017, entitled “Benzimidazole derivatives and uses thereof”; U.S. Ser. No. 11/382,897B2, published Jul. 12, 2022, entitled “Therapeutic combination for treatment of cerebellar ataxia”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating SCA2 by modulating the activity of genes and gene products other than ATXN2 genes/gene products, such as other genes/gene products associated with SCA and/or SCA2.

### Gene Therapies

ATXN2 expression and/or activity in some embodiments can be modulated by the use of gene therapies that can modulate ATXN2 (e.g., by delivery of nucleic acids encoding ATXN2 or other molecules that interact with ATXN2).

In some embodiments, gene therapies, such as those involving administration of a compounds encoding useful therapeutic agents, useful in the treatment of SCA2 are provided in US Patent Application Publication No. 20110142789A1, published Jun. 16, 2011, entitled “Compositions and Methods for the Diagnosis and Treatment of Amyotrophic Lateral Sclerosis”; US Patent Application Publication No. 20100047261A1, published Feb. 25, 2010, entitled “Base-modified RNA for increasing the expression of a protein”; US Patent Application Publication No. 20100203076A1, published Aug. 12, 2010, entitled “Complexes of RNA and cationic peptides for transfection and for immunostimulation”; U.S. Pat. No. 10,815,463B2, published Oct. 27, 2020, entitled “Messenger UNA molecules and uses thereof”; U.S. Pat. No. 11,155,817B2, published Oct. 26, 2021, entitled “Therapeutic for treatment of diseases including the central nervous system”; US Patent Application Publication No. 20180327471A1, published Nov. 15, 2018, entitled “Translatable molecules and synthesis thereof”; U.S. Ser. No. 11/559,588B2, published Jan. 24, 2023, entitled “Materials and methods for treatment of Spinocerebellar Ataxia Type 1 (SCA1) and other Spinocerebellar Ataxia Type 1 Protein (ATXN1) gene related conditions or disorders”; US20110016540A1, published Jan. 20, 2011, entitled “Genome editing of genes associated with trinucleotide repeat expansion disorders in animals”; US20210238226A1, published Aug. 5, 2021, entitled “Methods and compounds for the treatment of genetic disease”; the entire contents of each of which are herein incorporated by reference.

Certain gene therapies provided in this section may be useful in treating SCA2 by modulating the activity of genes and gene products other than ATXN2 genes/gene products, such as other genes/gene products associated with SCA and/or SCA2.

### Molecular Payloads Targeting ATXN3

The ATXN3 gene, which encodes the ataxin-3 protein, and mutations therein, are implicated in SCA type 3 (SCA3). Modulation of ATXN3 expression and activity (e.g., by suppressing the expression and/or activity of mutant ATXN3 protein and/or its interactions with other proteins) therefore in some embodiments can have a therapeutic effect in subjects with SCA3.

### Oligonucleotides

ATXN3 (and/or ataxin-3 protein encoded by ATXN3) expression and/or activity in some embodiments can be modulated by the use of oligonucleotides targeting ATXN3 sequences.

In some embodiments, an oligonucleotide useful for the treatment of SCA, e.g., targeting (e.g., directly or indirectly modulating the expression or activity of) ATXN3, comprises a region of complementarity to a ATXN3 transcript provided in Table 3, e.g., provided by any one of SEQ ID NOs: 821-832.

In some embodiments, examples of oligonucleotides useful for the treatment of SCA3, e.g., targeting (e.g., directly or indirectly modulating the expression or activity of) ATXN2, are provided in Miller, et al. “Allele-specific silencing of dominant disease genes” Proc. Nat. Acad. Sci. 100: 7195-7200 (2003); U.S. Ser. No. 11/345,915B2, published May 31, 2022, entitled “RNA modulating oligonucleotides with improved characteristics for the treatment of neuromuscular disorders”; US20220195431A1, published Jun. 23, 2022, entitled “Compounds and methods for reducing atxn3 expression”; U.S. Ser. No. 11/434,488B2, published Sep. 6, 2022, entitled “Compounds and methods for reducing ATXN3 expression”; US20230054720A1, published Feb. 23, 2023, entitled “Antisense Oligonucleotides Targeting ATXN3”; US20150315595A1, published Nov. 5, 2015, entitled “Compositions and Methods for Modulation of ATXN3 Expression”; US20230056569A1, published Feb. 23, 2023, entitled “Ataxin3 (atxn3) rnai agent compositions and methods of use thereof”; US20220010314A1, published Jan. 13, 2022, entitled “Rnai induced reduction of ataxin-3 for the treatment of spinocerebellar ataxia type 3”; U.S. Ser. No. 11/293,025B2, published Apr. 5, 2022, entitled “Compositions and methods for modulating Ataxin 3 expression”; U.S. Pat. No. 8,778,904B2, published Jul. 15, 2014, entitled “Methods and compositions for treating diseases, disorders or injury of the CNS”; the entire contents of each of which are herein incorporated by reference.

Certain oligonucleotides provided in this section may be useful in treating SCA3 by modulating the activity of genes and/or gene products other than ATXN3 genes/gene products, such as other genes/gene products associated with SCA and/or SCA3.

### Polypeptides

ATXN3 expression and/or activity in some embodiments can be modulated by the use of ataxin-3 polypeptides or polypeptides that can interact with ataxin-3 (e.g., to modulate its biological activity and/or its interaction with other biomolecules).

In some embodiments, examples of polypeptides (e.g., peptides; proteins, such as enzymes; antibodies; etc.) useful in the treatment of SCA3 are provided in U.S. Ser. No. 10/973,812B2, published Apr. 13, 2021, entitled “Ataxia therapeutic compositions and methods”; the entire contents of which are herein incorporated by reference.

Certain polypeptides provided in this section may be useful in treating SCA3 by modulating the activity of genes and gene products other than ATXN3 genes/gene products, such as other genes/gene products associated with SCA and/or SCA3.

### Small Molecules

ATXN3 expression and/or activity in some embodiments can be modulated by the use of small molecules that can modulate ATXN3 (e.g., to modulate its biological activity, its expression, and/or its interaction with other biomolecules).

In some embodiments, examples of small molecules useful in the treatment of SCA3 are provided in U.S. Ser. No. 11/382,897B2, published Jul. 12, 2022, entitled “Therapeutic combination for treatment of cerebellar ataxia”; the entire contents of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating SCA3 by modulating the activity of genes and gene products other than ATXN3 genes/gene products, such as other genes/gene products associated with SCA and/or SCA3.

### Gene Therapies

ATXN3 expression and/or activity in some embodiments can be modulated by the use of gene therapies that can modulate ATXN3 (e.g., by delivery of nucleic acids encoding ATXN3 or other molecules that interact with ATXN3).

In some embodiments, gene therapies, such as those involving administration of a compounds encoding useful therapeutic agents, useful in the treatment of SCA3 are provided in US20110016540A1, published Jan. 20, 2011, entitled “Genome editing of genes associated with trinucleotide repeat expansion disorders in animals”; US20210238226A1, published Aug. 5, 2021, entitled “Methods and compounds for the treatment of genetic disease”; WO2018002886A1, published Jan. 4, 2018, entitled “Materials and methods for treatment of spinocerebellar ataxia 3 (sca3) and other related disorders”; the entire contents of each of which are herein incorporated by reference.

Certain gene therapies provided in this section may be useful in treating SCA3 by modulating the activity of genes and gene products other than ATXN3 genes/gene products, such as other genes/gene products associated with SCA and/or SCA3.

### Molecular Payloads Targeting MSH3

The MSH3 gene, which encodes MutS Homolog 3 protein, and its overexpression has been implicated in spinocerebellar ataxia (SCA), which affects nerves in the central nervous system including the brain. Modulation of MSH3 and/or MutS Homolog 3 expression and activity (e.g., by suppressing the expression of MSH3 and/or activity of the encoded protein) therefore in some embodiments can have a therapeutic effect in subjects with SCA.

### Oligonucleotides

MSH3 expression and/or activity in some embodiments can be modulated by the use of oligonucleotides targeting MSH3 sequences.

In some embodiments, an oligonucleotide useful for the treatment of SCA, e.g., targeting (e.g., directly or indirectly modulating the expression or activity of) MSH3, comprises a region of complementarity to a MSH3 transcript provided in Table 3, e.g., provided by SEQ ID NO: 437.

In some embodiments, examples of oligonucleotides useful for the treatment of SCA, e.g., targeting (e.g., directly or indirectly modulating the expression or activity of) MSH3, are provided in International PCT Application Publication No. WO2021252799A2, published Dec. 16, 2021, entitled “Compounds and methods for reducing msh3 expression”; U.S. Pat. No. 10,669,542B2, published Jun. 2, 2020, entitled “Compositions and uses for treatment thereof”; US Patent Application Publication No. 20210269881A1, published Sep. 2, 2021, entitled “Long non-coding rnas (lncrnas) for the diagnosis and therapeutics of brain disorders, in particular cognitive disorders”; US Patent Application Publication No. 20220072028A1, published Mar. 10, 2022, entitled “Methods for the treatment of trinucleotide repeat expansion disorders associated with msh3 activity”; US Patent Application Publication No. 20210355491A1, published Nov. 18, 2021, entitled “Oligonucleotides for msh3 modulation”; International PCT Application Publication No. WO2021226549A1, published Nov. 11, 2021, entitled “Methods for the treatment of nucleotide repeat expansion disorders associated with msh3 activity”; International PCT Application Publication No. WO2021247020A1, published Dec. 9, 2021, entitled “Methods for the treatment of nucleotide repeat expansion disorders associated with msh3 activity”; US Patent Application Publication No. 20210395740A1, published Dec. 23, 2021, entitled “Methods for the treatment of nucleotide repeat expansion disorders associated with msh3 activity”; the entire contents of each of which are herein incorporated by reference.

Certain oligonucleotides provided in this section may be useful in treating SCA by modulating the activity of genes and/or gene products other than MSH3 genes/gene products, such as other genes/gene products associated with SCA.

### Polypeptides

MSH3 expression and/or activity in some embodiments can be modulated by the use of MSH3 polypeptides or polypeptides that can interact with MSH3 (e.g., to modulate its biological activity and/or its interaction with other biomolecules).

In some embodiments, examples of polypeptides (e.g., peptides; proteins, such as enzymes; antibodies; etc.) useful in the treatment of SCA are provided in U.S. Ser. No. 10/973,812B2, published Apr. 13, 2021, entitled “Ataxia therapeutic compositions and methods”; the entire contents of which are herein incorporated by reference.

Certain polypeptides provided in this section may be useful in treating SCA by modulating the activity of genes and gene products other than MSH3 genes/gene products, such as other genes/gene products associated with SCA.

### Small Molecules

MSH3 expression and/or the activity of the protein it encodes in some embodiments can be modulated by the use of small molecules that can modulate MSH3 (e.g., to modulate its biological activity, its expression, its localization, and/or its interaction with other biomolecules).

In some embodiments, examples of small molecules useful in the treatment of SCA are provided in U.S. Pat. No. 8,623,600B2, published Jan. 7, 2014, entitled “Methods and compositions for identifying inhibitors of MutSα or MutSβ interaction with MutLα”; US Patent Application Publication No. 20210283114A1, published Sep. 16, 2021, entitled “Methods of treating diseases associated with repeat instability”; International PCT Application Publication No. WO2021174167A1, published Sep. 2, 2021, entitled “Compounds and methods for modulating splicing”; U.S. Pat. No. 8,105,836B2, published Jan. 31, 2012, entitled “Chemical inhibitors of mismatch repair”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating SCA by modulating the activity of genes and gene products other than MSH3 genes/gene products, such as other genes/gene products associated with SCA.

### Gene Therapies

MSH3 expression and/or activity in some embodiments can be modulated by the use of gene therapies that can modulate MSH3 (e.g., by delivery of nucleic acids encoding MSH3 or other molecules that interact with MSH3 transcripts or the protein encoded by MSH3).

In some embodiments, gene therapies, such as those involving administration of compounds encoding useful therapeutic agents, useful in the treatment of SCA are provided in U.S. Pat. No. 8,674,179B2, published Mar. 18, 2014, entitled “Modifying the DNA recombination potential in eukaryotes”; the entire contents of which are herein incorporated by reference.

Certain gene therapies provided in this section may be useful in treating SCA by modulating the activity of genes and gene products other than MSH3 genes/gene products, such as other genes/gene products associated with SCA.

### Molecular Payloads for the Treatment of Huntington's Disease

Various molecular payloads may be useful in the treatment of Huntington's disease, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of Huntington's disease may include, in some embodiments, molecular payloads which modulate (e.g., increase or decrease) expression or activity of HTT, mHTT, and/or MSH3.

Examples of oligonucleotides useful for the treatment of Huntington's disease, e.g., oligonucleotides targeting (e.g., directly or indirectly modulating the expression or activity of) genes associated with Huntington's disease (e.g., HTT, mHTT, MSH3, etc.), include those listed in Table 8 below. Each oligonucleotide provided in Table 8 may have any modification pattern disclosed herein.

TABLE 8
Oligonucleotides for the treatment of Huntington′s disease

| | | Oligo- | |
|---|---|---|---|
| SEQ | | nucleotide | |
| ID | Target | Sequence | |
| NO: | gene | (5′-3′) | Oligonucleotide Structure (5′-3′) |
| 53 | HTT | GGCACAAGGG | mG*SmGmCmAmC*SA*SA*SG*SG*SG*SC*SA*SC*RA*SG*SmAmCm |
| | | CACAGACUUC | UmU*SmC, wherein: |
| | | | *S represents a Sp phosphorothioate; |
| | | | *R represents a Rp phosphorothioate; and |
| | | | m represents a 2′-OMe modified nucleoside. |
| 54 | HTT | GUGCACACAG | mG*SmUmGmCmA*SC*SA*SC*SA*SG*ST*SA*SG*RA*ST*SmGmAm |
| | | TAGATGAGGG | GmG*SmG, wherein: |
| | | | *S represents a Sp phosphorothioate; |
| | | | *R represents a Rp phosphorothioate; and |
| | | | m represents a 2′-OMe modified nucleoside. |
| 55 | HTT | GCTGCTGCTG | G C lT G C lT G C lT G C lT G C lT G C lT G C lT |
| | | CTGCTGCTGC | G, wherein l represents an LNA nucleoside, and the |
| | | TG | oligonucleotide has a DNA backbone |
| 56 | HTT | GCTGCTGCTG | G C T G C T G C T G C T G C T G C T G C T G, |
| | | CTGCTGCTGC | wherein the oligonucleotide is a phosphorodiamidate |
| | | TG | morpholino oligomer |
| 57 | HTT | GCTATTACCT | G C T A T T A C C T T A A C C C A G, wherein the |
| | | TAACCCAG | oligonucleotide is a phosphorodiamidate morpholino |
| | | | oligomer |
| 58 | HTT | ACCTTATCTG | Aes mCeo mCeo Teo Tes Ads Tds mCds Tds Gds mCds |
| | | CACGGTTG | Ads mCds Geo Geo Teo Tes Ge, wherein mC = 5-methyl |
| | | | cytosine, e = 2′-MOE modified sugar, d = 2′deoxyribose |
| | | | sugar, s = phosphorothioate internucleoside linkage, |
| | | | o = phosphodiester internucleoside linkage |
| 59 | HTT | CTCAGTAACA | mCes Teo mCeo Aeo Ges Tds Ads Ads mCds Ads Tds |
| | | TTGACACCAC | Tds Gds Ads mCds Aeo mCeo mCeo Aes mCe, wherein |
| | | | mC = 5-methyl cytosine, e = 2′-MOE modified sugar, |
| | | | d = 2′deoxyribose sugar, s = phosphorothioate |
| | | | internucleoside linkage, o = phosphodiester |
| | | | internucleoside linkage |
| 60 | HTT | CTCAGTAACA | mCes Tes mCes Aes Ges Tds Ads Ads mCds Ads Tds |
| | | TTGACACCAC | Tds Gds Ads mCds Aes mCes mCes Aes mCe, wherein e = |
| | | | 2′-MOE modified sugar, d = 2′deoxyribose sugar, s = |
| | | | phosphorothioate internucleoside linkage |
| 61 | HTT | CTCGACTAAA | mCes Tes mCes Ges Aes mCds Tds Ads Ads Ads Gds |
| | | GCAGGATTTC | mCds Ads Gds Gds Aes Tes Tes Tes mCes, wherein e = |
| | | | 2′-MOE modified sugar, d = 2′deoxyribose sugar, s = |
| | | | phosphorothioate internucleoside linkage |
| 62 | HTT | CCTTCCCTGA | mCes mCes Tes Tes mCes mCds mCds Tds Gds Ads Ads |
| | | AGGTTCCTCC | Gds Gds Tds Tds mCes mCes Tes mCes mCes, wherein e = |
| | | | 2′-MOE modified sugar, d = 2′deoxyribose sugar, s = |
| | | | phosphorothioate internucleoside linkage |
| 63 | HTT | GCAGGGTTAC | Ges mCes Aes Ges Ges Gds Tds Tds Ads mCds mCds |
| | | CGCCATCCCC | Gds mCds mCds Ads Tes mCes mCes mCes mCes, wherein |
| | | | e = 2′-MOE modified sugar, d = 2′deoxyribose sugar, s = |
| | | | phosphorothioate internucleoside linkage |
| 64 | HTT | CGAGACAGTC | mCes Ges Aes Ges Aes mCds Ads Gds Tds mCds Gds |
| | | GCTTCCACTT | mCds Tds Tds mCds mCes Aes mCes Tes Tes, wherein e = |
| | | | 2′-MOE modified sugar, d = 2′deoxyribose sugar, s = |
| | | | phosphorothioate internucleoside linkage |
| 65 | HTT | CTCAGTAACA | mCes Teo mCeo Aeo Ges Tds Ads Ads mCds Ads Tds |
| | | TTGACACCAC | Tds Gds Ads mCds Aeo mCeo mCeo Aes mCe, wherein e = |
| | | | 2′-MOE modified sugar, d = 2′deoxyribose sugar, s = |
| | | | phosphorothioate internucleoside linkage, o = |
| | | | phosphodiester internucleoside linkage |
| | HTT | | siRNA with a sequence having complementarity to |
| | | | UAAGAGAUGGGGACAGUACUUCAACGCUAGAAGAACA (SEQ ID NO: |
| | | | 66) |

In each sequence listed in Table 8, each T may be optionally and independently replaced with a U, and each U may be optionally and independently replaced with a T.

Examples of small molecules useful for the treatment of Huntington's disease include:

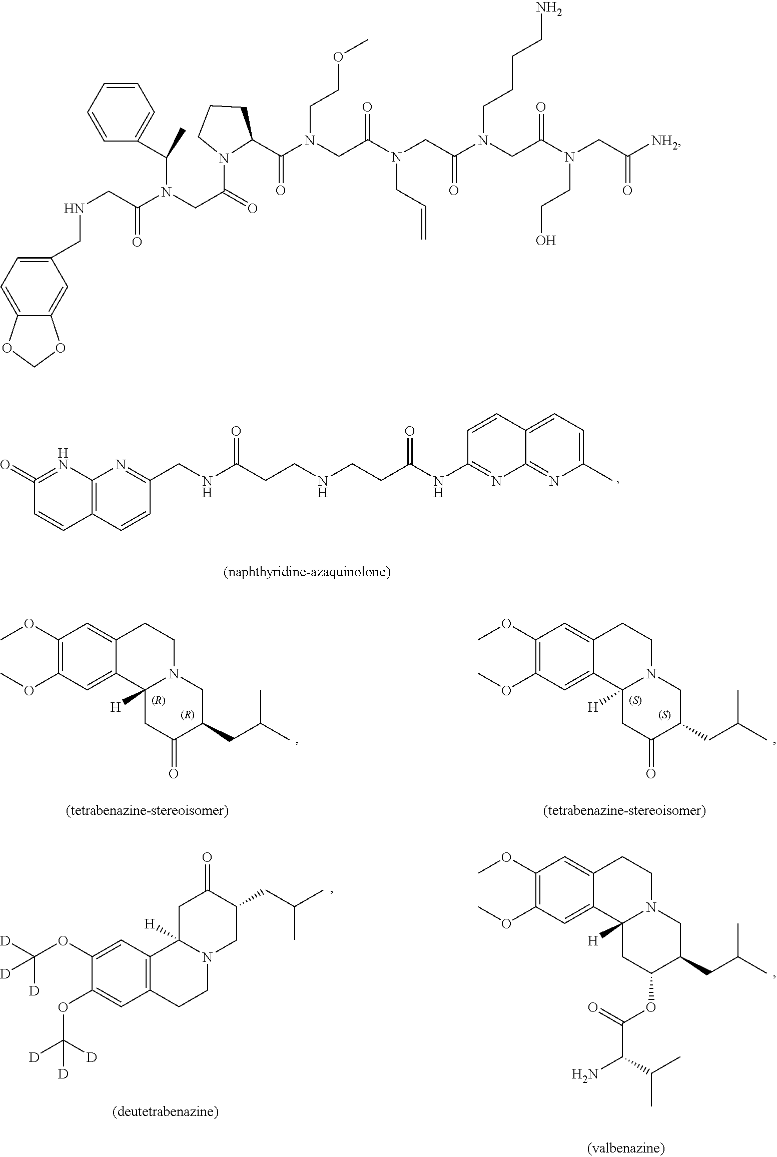

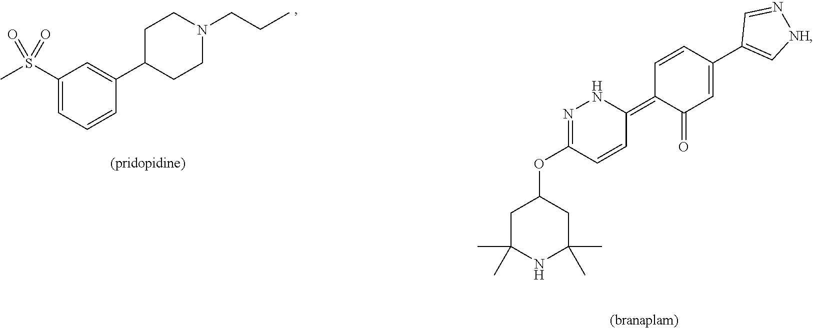

PTC518, and pharmaceutically acceptable salts, co-crystals, tautomers, stereoisomers, solvates, hydrates, polymorphs, isotopically enriched compounds, and prodrugs thereof.

Examples of polypeptides useful for the treatment of Huntington's disease include antibodies, proteins, peptides, and enzymes.

### Molecular Payloads Targeting HTT

The HTT gene, which encodes huntingtin protein, and mutations therein are implicated in Huntington's disease, which affects nerves in the brain. Modulation of HTT and/or mHTT expression and activity (e.g., by suppressing the expression of mutant HTT and/or activity of the huntingtin protein) therefore in some embodiments can have a therapeutic effect in subjects with Huntington's disease.

### Oligonucleotides

HTT expression and/or activity in some embodiments can be modulated by the use of oligonucleotides targeting HTT sequences.

In some embodiments, an oligonucleotide useful for the treatment of Huntington's disease, e.g., targeting (e.g., directly or indirectly modulating the expression or activity of) HTT, comprises a region of complementarity to an HTT transcript provided in Table 3, e.g., provided by any one of SEQ ID NOs: 435-436.

In some embodiments, examples of oligonucleotides useful for the treatment of Huntington's disease, e.g., targeting (e.g., directly or indirectly modulating the expression or activity of) HTT, are provided in Miller, et al. “Allele-Selective Suppression of Mutant Huntingtin in Primary Human Blood Cells” Scientific Reports 7, article 46740 (2017); Kordasiewicz, H. B. et al. (2012) “Sustained therapeutic reversal of Huntington's disease by transient repression of huntingtin synthesis” Neuron 74:1031-1044; US Patent Application Publication No. 20140142288A1, published Nov. 10, 2015, entitled “Therapeutic compounds”; US Patent Application Publication No. 20150148404A1, published May 28, 2015, entitled “RNA Modulating Oligonucleotides with Improved Characteristics for the Treatment of Neuromuscular Disorders”; U.S. Pat. No. 10,066,228B2, published Sep. 4, 2018, entitled “Oligonucleotides for treating expanded repeat diseases”; U.S. Pat. No. 11,345,915B2, published May 31, 2022, entitled “RNA modulating oligonucleotides with improved characteristics for the treatment of neuromuscular disorders”; U.S. Pat. No. 11,118,179B2, published Sep. 14, 2021, entitled “Mixed tricyclo-DNA, 2′-modified RNA oligonucleotide compositions and uses thereof”; US Patent Application Publication No. 20210169914A1, published Jun. 10, 2021, entitled “Nucleic acids and nucleic acid analogs for treating, preventing, and disrupting pathological polynucleotide-binding protein inclusions”; U.S. Pat. No. 10,479,995B2, published Nov. 19, 2019, entitled “Oligonucleotide compositions and methods thereof”; U.S. Pat. No. 10,724,035B2, published Jul. 28, 2020, entitled “Oligonucleotide compositions and methods thereof”; US Patent Application Publication No. 20220162598A1, published May 26, 2022, entitled “Oligonucleotide compositions and methods thereof”; International PCT Application Publication No. WO2021168183A1, published Aug. 26, 2021, entitled “Methods for reducing HTT expression”; U.S. Pat. No. 9,273,315B2, published Mar. 1, 2016, entitled “Modulation of huntingtin expression”; US Patent Application Publication No. 20220042013A1, published Feb. 10, 2022, entitled “Compositions and their uses directed to huntingtin”; US Patent Application Publication No. 20030109476A1, published Jun. 12, 2003, entitled “Compositions and methods for the prevention and treatment of Huntington's disease”; US Patent Application Publication No. 20040009899A1, published Jan. 15, 2004, entitled “Treating dominant disorders”; U.S. Pat. No. 11,299,734B2, published Apr. 12, 2022, entitled “RNA interference for the treatment of gain-of-function disorders”; U.S. Pat. No. 8,557,975B2, published Oct. 15, 2013, entitled “Methods and sequences to suppress primate Huntington gene expression”; U.S. Pat. No. 10,093,927B2, published Oct. 9, 2018, entitled “Reduction of off-target RNA interference toxicity”; U.S. Pat. No. 10,837,016B2, published Nov. 17, 2020, entitled “Modulation of huntingtin expression”; U.S. Pat. No. 9,574,191B2, published Feb. 21, 2017, entitled “Selective inhibition of polyglutamine protein expression”; U.S. Pat. No. 9,523,093B2, published Dec. 20, 2016, entitled “Huntington's disease therapeutic compounds”; the entire contents of each of which are herein incorporated by reference.

Certain oligonucleotides provided in this section may be useful in treating Huntington's by modulating the activity of genes and/or gene products other than HTT genes/gene products, such as MSH3 genes/gene products.

### Polypeptides

HTT expression and/or activity of the protein encoded thereby in some embodiments can be modulated by the use of polypeptides, such as polypeptides that can interact with HTT nucleic acids and/or its encoded protein (e.g., to modulate its biological activity, its localization within the cell, and/or its interaction with other biomolecules).

In some embodiments, examples of polypeptides (e.g., peptides; proteins, such as enzymes; antibodies; etc.) useful in the treatment of Huntington's disease are provided in U.S. Pat. No. 7,375,194B2, published May 20, 2008, entitled “Antibodies that bind to an epitope on the Huntington's disease protein”; US Patent Application Publication No. 20040009899A1, published Jan. 15, 2004, entitled “Treating dominant disorders”; U.S. Pat. No. 8,673,852B2, entitled “Methods of treating neuronal disorders using MNTF peptides and analogs thereof”; U.S. Pat. No. 10,040,836B2, published Aug. 7, 2018, entitled “Peptides for the treatment of neurodegenerative diseases”; U.S. Pat. No. 11,072,650B2, published Jul. 27, 2021, entitled “Single chain intrabodies that alter Huntingtin mutant degradation”; U.S. Pat. No. 10,556,946B2, published Feb. 11, 2020, entitled “Human derived anti-Huntingtin (HTT) antibodies and uses thereof”; the entire contents of each of which are herein incorporated by reference.

Certain polypeptides provided in this section may be useful in treating Huntington's disease by modulating the activity of genes and gene products other than HTT genes/gene products, such as MSH3 genes/gene products.

### Small Molecules

HTT expression and/or the activity of the protein it encodes in some embodiments can be modulated by the use of small molecules that can modulate HTT (e.g., to modulate its biological activity, its expression, its localization, and/or its interaction with other biomolecules).

In some embodiments, examples of small molecules useful in the treatment of Huntington's disease are provided in U.S. Pat. No. 8,729,263B2, published May 20, 2014, entitled “1,4-disubstituted pyridazine analogs there of and methods for treating SMN-deficiency-related conditions”; U.S. Pat. No. 10,874,672B2, published Dec. 29, 2020, entitled “Methods for treating Huntington's disease”; US Patent Application Publication No. 20130303562A1, published Nov. 14, 2013, entitled “Chemical and RNAi suppressors of neurotoxicity in Huntington's disease”; International PCT Patent Application Publication No. WO2013043669A1, published Mar. 28, 2013, entitled “Peptoid compositions for the treatment of Alzheimer's disease and polyglutamine expansion disorder”; International PCT Patent Application Publication No. WO2021174167A1, published Sep. 2, 2021, entitled “Compounds and methods for modulating splicing”; US Patent Application Publication No. 20040229837A1, published Nov. 18, 2004, entitled “Treatment of neurodegenerative diseases”; US Patent Application Publication No. 20100022637A1, published Jan. 28, 2010, entitled “Identification of anti-cancer compounds and compounds for treating huntington's disease and methods of treatment thereof”; US Patent Application Publication No. 20100069372A1, published Mar. 18, 2010, entitled “Compositions and methods for modulating poly(adp-ribose) polymerase activity”; US Patent Application Publication No. 20080300178A1, published Dec. 4, 2008, entitled “Method For Treating Huntington's Disease by Inhibiting Dephosphorylation of Huntingtin at S421”; US Patent Application Publication No. 20130303562A1, published Nov. 14, 2013, entitled “Chemical and rnai suppressors of neurotoxicity in huntington's disease”; International PCT Application Publication No. WO2013043669A1, published Mar. 28, 2013, entitled “Peptoid compositions for the treatment of alzheimer's disease and polyglutamine expansion disorder”; International PCT Application Publication No. WO2021174167A1, published Sep. 2, 2021, entitled “Compounds and methods for modulating splicing”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Huntington's disease by modulating the activity of genes and gene products other than HTT genes/gene products, such as MSH3 genes/gene products.

### Gene Therapies

HTT expression and/or activity in some embodiments can be modulated by the use of gene therapies that can modulate HTT (e.g., by delivery of nucleic acids encoding HTT or other molecules that interact with HTT transcripts or the protein encoded by HTT).

In some embodiments, gene therapies, such as those involving administration of a compounds encoding useful therapeutic agents, useful in the treatment of Huntington's disease are provided in US Patent Application Publication No. 20140142288A1, published Nov. 10, 2015, entitled “Therapeutic compounds”; US Patent Application Publication No. 20180327471A1, published Nov. 15, 2018, entitled “Translatable molecules and synthesis thereof”; US Patent Application Publication No. 20130164845A1, published Jun. 27, 2013, entitled “Compositions and Methods for the Delivery of Biologically Active RNAs”; U.S. Pat. No. 11,110,154B2, published Sep. 7, 2021, entitled “Methods and compositions for treating Huntington's Disease”; U.S. Pat. No. 9,523,093B2, published Dec. 20, 2016, entitled “Huntington's disease therapeutic compounds”; the entire contents of each of which are herein incorporated by reference.

Certain gene therapies provided in this section may be useful in treating Huntington's disease by modulating the activity of genes and gene products other than HTT genes/gene products, such as MSH3 genes/gene products.

### Molecular Payloads Targeting MSH3

The MSH3 gene, which encodes MutS Homolog 3 protein, and its overexpression has been implicated in Huntington's disease, which affects nerves in the brain. Modulation of MSH3 and/or MutS Homolog 3 expression and activity (e.g., by suppressing the expression of MSH3 and/or activity of the encoded protein) therefore in some embodiments can have a therapeutic effect in subjects with Huntington's disease.

### Oligonucleotides

MSH3 expression and/or activity in some embodiments can be modulated by the use of oligonucleotides targeting MSH3 sequences.

In some embodiments, an oligonucleotide useful for the treatment of Huntington's disease, e.g., targeting (e.g., directly or indirectly modulating the expression or activity of) MSH3, comprises a region of complementarity to a MSH3 transcript provided in Table 3, e.g., provided by SEQ ID NO: 437.

In some embodiments, examples of oligonucleotides useful for the treatment of Huntington's disease, e.g., targeting (e.g., directly or indirectly modulating the expression or activity of) MSH3, are provided in International PCT Application Publication No. WO2021252799A2, published Dec. 16, 2021, entitled “Compounds and methods for reducing msh3 expression”; U.S. Pat. No. 10,669,542B2, published Jun. 2, 2020, entitled “Compositions and uses for treatment thereof”; US Patent Application Publication No. 20210269881A1, published Sep. 2, 2021, entitled “Long non-coding rnas (lncrnas) for the diagnosis and therapeutics of brain disorders, in particular cognitive disorders”; US Patent Application Publication No. 20220072028A1, published Mar. 10, 2022, entitled “Methods for the treatment of trinucleotide repeat expansion disorders associated with msh3 activity”; US Patent Application Publication No. 20210355491A1, published Nov. 18, 2021, entitled “Oligonucleotides for msh3 modulation”; International PCT Application Publication No. WO2021226549A1, published Nov. 11, 2021, entitled “Methods for the treatment of nucleotide repeat expansion disorders associated with msh3 activity”; International PCT Application Publication No. WO2021247020A1, published Dec. 9, 2021, entitled “Methods for the treatment of nucleotide repeat expansion disorders associated with msh3 activity”; US Patent Application Publication No. 20210395740A1, published Dec. 23, 2021, entitled “Methods for the treatment of nucleotide repeat expansion disorders associated with msh3 activity”; the entire contents of each of which are herein incorporated by reference.

Certain oligonucleotides provided in this section may be useful in treating Huntington's by modulating the activity of genes and/or gene products other than MSH3 genes/gene products, such as HTT genes/gene products.

### Polypeptides

MSH3 expression and/or activity of the protein encoded thereby in some embodiments can be modulated by the use of polypeptides, such as polypeptides that can interact with MSH3 nucleic acids and/or its encoded protein (e.g., to modulate its biological activity, its localization within the cell, and/or its interaction with other biomolecules).

### Small Molecules

MSH3 expression and/or the activity of the protein it encodes in some embodiments can be modulated by the use of small molecules that can modulate MSH3 (e.g., to modulate its biological activity, its expression, its localization, and/or its interaction with other biomolecules).

In some embodiments, examples of small molecules useful in the treatment of Huntington's disease are provided in U.S. Pat. No. 8,623,600B2, published Jan. 7, 2014, entitled “Methods and compositions for identifying inhibitors of MutSα or MutSβ interaction with MutLα”; US Patent Application Publication No. 20210283114A1, published Sep. 16, 2021, entitled “Methods of treating diseases associated with repeat instability”; International PCT Application Publication No. WO2021174167A1, published Sep. 2, 2021, entitled “Compounds and methods for modulating splicing”; U.S. Pat. No. 8,105,836B2, published Jan. 31, 2012, entitled “Chemical inhibitors of mismatch repair”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Huntington's disease by modulating the activity of genes and gene products other than MSH3 genes/gene products, such as HTT genes/gene products.

### Gene Therapies

MSH3 expression and/or activity in some embodiments can be modulated by the use of gene therapies that can modulate MSH3 (e.g., by delivery of nucleic acids encoding MSH3 or other molecules that interact with MSH3 transcripts or the protein encoded by MSH3).

In some embodiments, gene therapies, such as those involving administration of compounds encoding useful therapeutic agents, useful in the treatment of Huntington's disease are provided in U.S. Pat. No. 8,674,179B2, published Mar. 18, 2014, entitled “Modifying the DNA recombination potential in eukaryotes”; the entire contents of which are herein incorporated by reference.

Certain gene therapies provided in this section may be useful in treating Huntington's disease by modulating the activity of genes and gene products other than MSH3 genes/gene products, such as HTT genes/gene products.

### Molecular Payloads for the Treatment of Parkinson's Disease

Various molecular payloads may be useful in the treatment of Parkinson's disease, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of Parkinson's disease may include, in some embodiments, molecular payloads which modulate (e.g., increase or decrease) expression or activity of LRRK2 and/or SNCA.

Examples of oligonucleotides useful for the treatment of Parkinson's disease, e.g., oligonucleotides targeting (e.g., directly or indirectly modulating the expression or activity of) genes associated with Parkinson's disease (e.g., LRRK2, SNCA, etc.), include those listed in Table 9 below. Each oligonucleotide provided in Table 9 may have any modification pattern disclosed herein.

TABLE 9
Oligonucleotides for the treatment of Parkinson′s disease

| SEQ | | | |
|---|---|---|---|
| ID | Target | Oligonucleotide | |
| NO: | gene | Sequence (5′-3′) | Oligonucleotide Structure (5′-3′) |
| 67 | LRRK2 | GGACTGCTCTCTT | Ges Ges Aes mCes Tes Gds mCds Tds mCds Tds |
| | | TCTCACA | mCds Tds Tds Tds mCds Tes mCes Aes mCes |
| | | | Ae, wherein mC = 5-methyl cytosine, e = 2′-MOE |
| | | | modified sugar, d = 2′deoxyribose sugar, s = |
| | | | phosphorothioate internucleoside linkage |
| 68 | LRRK2 | TCCACATTTCTGA | Tes mCes mCes Aes mCes Ads Tds Tds Tds |
| | | ATCCCAG | mCds Tds Gds Ads Ads Tds mCes mCes mCes |
| | | | Aes Ge, wherein mC = 5-methyl cytosine, e = 2′- |
| | | | MOE modified sugar, d = 2′deoxyribose sugar, s = |
| | | | phosphorothioate internucleoside linkage |
| 69 | LRRK2 | GGACTGCTCTCTT | Ges Geo Aeo mCeo Tes Gds mCds Tds mCds Tds |
| | | TCTCACA | mCds Tds Tds Tds mCds Teo mCeo Aes mCes |
| | | | Ae, wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage |
| 70 | LRRK2 | TCCACATTTCTGA | Tes mCeo mCeo Aeo mCes Ads Tds Tds Tds |
| | | ATCCCAG | mCds Tds Gds Ads Ads Tds mCeo mCeo mCes |
| | | | Aes Ge, wherein mC = 5-methyl cytosine, e = 2′- |
| | | | MOE modified sugar, d = 2′deoxyribose sugar, s = |
| | | | phosphorothioate internucleoside linkage, o = |
| | | | phosphodiester internucleoside linkage |
| 71 | LRRK2 | ACGCACTTAACAA | Aes mCeo Geo mCeo Aes mCds Tds Tds Ads Ads |
| | | TATCATA | mCds Ads Ads Tds Ads Teo mCeo Aes Tes Ae, |
| | | | wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage |
| 72 | LRRK2 | GCTCATATCTAAA | Ges mCeo Teo mCeo Aes Tds Ads Tds mCds Tds |
| | | GACCGCA | Ads Ads Ads Gds Ads mCeo mCeo Ges mCes Ae, |
| | | | wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage |
| 73 | LRRK2 | TCACCACAAACTC | Tes mCeo Aeo mCeo mCes Ads mCds Ads Ads |
| | | ATGGACT | Ads mCds Tds mCds Ads Tds Geo Geo Aes mCes |
| | | | Te, wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage |
| 74 | LRRK2 | ACCCTTTCCATGT | Aes mCeo mCeo mCeo Tes Tds Tds mCds mCds |
| | | GAACATT | Ads Tds Gds Tds Gds Ads Aeo mCeo Aes Tes |
| | | | Te, wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage |
| 75 | LRRK2 | AGCAATCATTGGT | Aes Geo mCeo Aeo Aes Tds mCds Ads Tds Tds |
| | | AGCATAC | Gds Gds Tds Ads Gds mCeo Aeo Tes Aes mCe, |
| | | | wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage |
| 76 | LRRK2 | CGCACTTAACAAT | mCes Geo mCes Aes mCes Tds Tds Ads Ads |
| | | ATCATAT | mCds Ads Ads Tds Ads Tds mCes Aeo Tes Aes |
| | | | Te, wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage |
| 77 | SNCA | ACAGATATTTTTG | Aes mCeo Aes Ges Aes Tds Ads Tds Tds Tds |
| | | TTCTGCC | Tds Tds Gds Tds Tds mCeo Teo Ges mCes mCe, |
| | | | wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage. |
| 78 | SNCA | CAGACTGTAATCT | mCes Aeo Geo Aeo mCes Tds Gds Tds Ads Ads |
| | | AGGACCC | Tds mCds Tds Ads Gds Geo Aeo mCes mCes |
| | | | mCe, wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage. |
| 79 | SNCA | ACGACATTTTCTT | Aes mCeo Geo Aeo mCes Ads Tds Tds Tds Tds |
| | | GCCTCTT | mCds Tds Tds Gds mCds mCeo Teo mCes Tes |
| | | | Te, wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage. |
| 80 | SNCA | GTTTTCATCAATA | Ges Teo Teo Teo Tes mCds Ads Tds mCds Ads |
| | | TCTGCAA | Ads Tds Ads Tds mCds Teo Geo mCes Aes Ae, |
| | | | wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage. |
| 81 | SNCA | TGAATTCCTTTAC | Tes Geo Aes Aes Tes Tds mCds mCds Tds Tds |
| | | ACCACAC | Tds Ads mCds Ads mCds mCes Aeo mCes Aes |
| | | | mCe, wherein mC = 5-methyl cytosine, e = 2′-MOE |
| | | | modified sugar, d = 2′deoxyribose sugar, s = |
| | | | phosphorothioate internucleoside linkage, o = |
| | | | phosphodiester internucleoside linkage. |
| 82 | SNCA | GTTTTCATCAATA | Ges Teo Teo Teo Tes mCds Ads Tds mCds Ads |
| | | TCTGCAA | Ads Tds Ads Tds mCds Teo Geo mCes Aes Ae, |
| | | | wherein mC indicates 5-methylcytosine, e indicates |
| | | | 2′-O-methoxyethylribose (MOE), d indicates 2′ |
| | | | deoxyribose sugar, s indicates phosphorothioate |
| | | | internucleoside linkage, and o indicates |
| | | | phosphodiester internucleoside linkage. |
| 83 | SNCA | ACAGATATTTTTG | Aes mCeo Aes Ges Aes Tds Ads Tds Tds Tds |
| | | TTCTGCC | Tds Tds Gds Tds Tds mCeo Teo Ges mCes mCe, |
| | | | wherein mC indicates 5-methylcytosine, e indicates |
| | | | 2′-O-methoxyethylribose (MOE), d indicates 2′ |
| | | | deoxyribose sugar, s indicates phosphorothioate |
| | | | internucleoside linkage, and o indicates |
| | | | phosphodiester internucleoside linkage. |

In each sequence listed in Table 9, each T may be optionally and independently replaced with a U.

Examples of small molecules useful for the treatment of Parkinson's disease include:

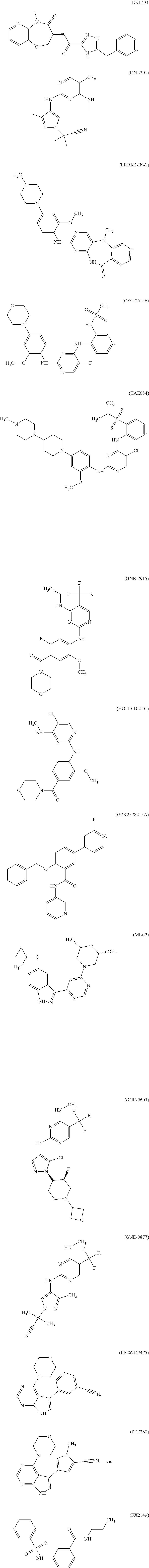

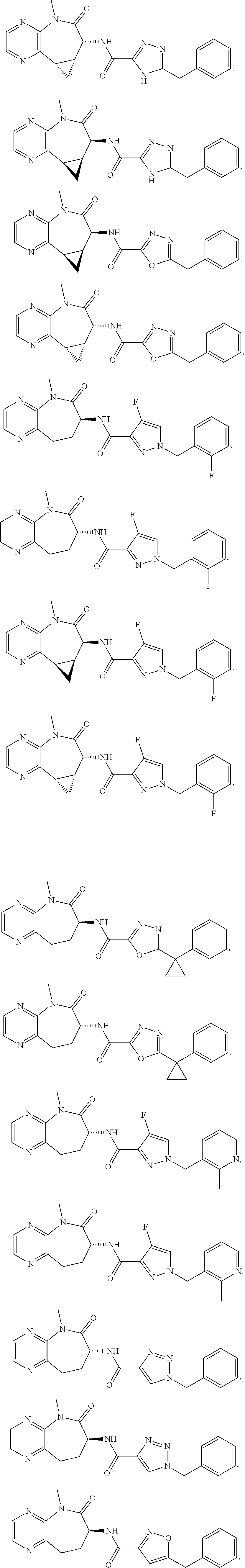

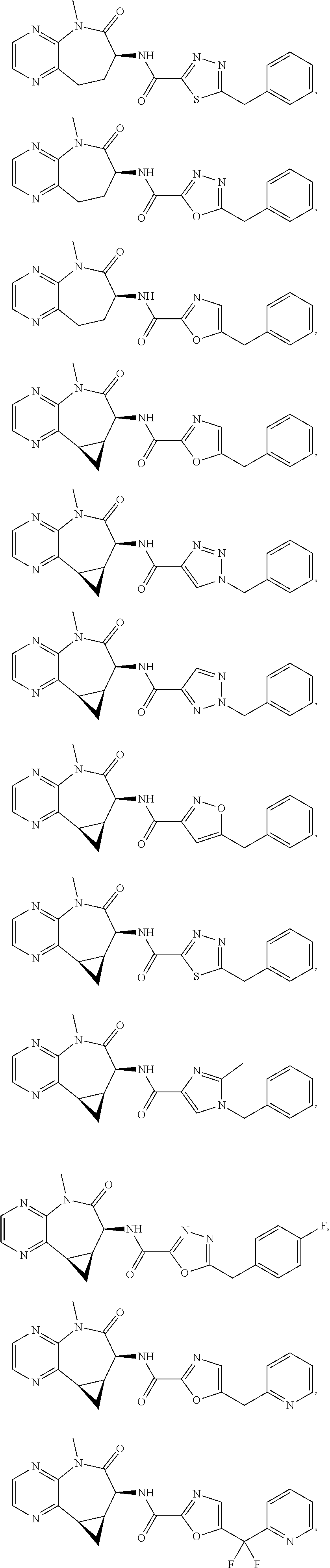

and pharmaceutically acceptable salts, co-crystals, tautomers, stereoisomers, solvates, hydrates, polymorphs, isotopically enriched compounds, and prodrugs thereof.

Examples of polypeptides useful for the treatment of Parkinson's disease include antibodies, proteins, peptides, and enzymes. In some embodiments, a polypeptide useful for the treatment of Parkinson's disease comprises an amino acid sequence: KGAEEMETVIPVDVMRRAGI (SEQ ID NO: 84), EGPYDVVVLPGGNLGAQNLS (SEQ ID NO: 85), or KGAEEMETVIPVD (SEQ ID NO: 86).

### Molecular Payloads Targeting LRRK2

The LRRK2 gene, which encodes dardarin protein (also known as leucine-rich repeat kinase 2 and PARK8), and mutations therein are implicated in Parkinson's disease, which affects nerves in the brain, primarily of the motor system. Modulation of LRRK2 expression and activity (e.g., by suppressing the expression of mutant LRRK2 and/or activity of the dardarin protein) therefore in some embodiments can have a therapeutic effect in subjects with Parkinson's disease.

### Oligonucleotides

LRRK2 expression and/or activity in some embodiments can be modulated by the use of oligonucleotides targeting LRRK2 sequences.

In some embodiments, an oligonucleotide useful for the treatment of Parkinson's disease, e.g., targeting (e.g., directly or indirectly modulating the expression or activity of) LRRK2, comprises a region of complementarity to an LRRK2 transcript provided in Table 3, e.g., provided by any one of SEQ ID NOs: 405-416.

In some embodiments, examples of oligonucleotides useful for the treatment of Parkinson's disease, e.g., targeting (e.g., directly or indirectly modulating the expression or activity of) LRRK2, are provided in Zhao, H. T. et al. (2017), “LRRK2 Antisense Oligonucleotides Ameliorate α-Synuclein Inclusion Formation in a Parkinson's Disease Mouse Model” Molecular Therapy—Nucleic Acids 8:508-519; U.S. Pat. No. 11,332,746B1, published May 17, 2022, entitled “Compounds and methods for reducing LRRK2 expression”; U.S. Pat. No. 10,907,160B2, published Feb. 2, 2021, entitled “Methods for reducing LRRK2 expression”; International PCT Application Publication No. WO2012131365A1, published Oct. 4, 2012, entitled “Therapeutic molecules for use in the suppression of parkinson's disease”; U.S. Pat. No. 10,724,035B2, published Jul. 28, 2020, entitled “Oligonucleotide compositions and methods thereof”; U.S. Pat. No. 10,787,669B2, published Sep. 29, 2020, entitled “Antisense compounds targeting Leucine-Rich repeat kinase 2(LRRK2) for the treatment of Parkinsons disease”; International PCT Application Publication No. WO2021150969A1, published Jul. 29, 2021, entitled “LEUCINE-RICH REPEAT KINASE 2 (LRRK2) iRNA AGENT COMPOSITIONS AND METHODS OF USE THEREOF”; the entire contents of each of which are herein incorporated by reference.

Certain oligonucleotides provided in this section may be useful in treating Parkinson's by modulating the activity of genes and/or gene products other than LRRK2 genes/gene products, such as SNCA genes/gene products.

### Polypeptides

LRRK2 expression and/or activity of the protein encoded thereby in some embodiments can be modulated by the use of polypeptides, such as polypeptides that can interact with LRRK2 nucleic acids and/or its encoded protein (e.g., to modulate its biological activity, its localization within the cell, and/or its interaction with other biomolecules).

In some embodiments, examples of polypeptides (e.g., peptides; proteins, such as enzymes; antibodies; etc.) useful in the treatment of Parkinson's disease are provided in U.S. Pat. No. 10,066,007B2, published Sep. 4, 2019, entitled “Dipeptide-repeat proteins as therapeutic target in neurodegenerative diseases with hexanucleotide repeat expansion”; U.S. U.S. Pat. No. 8,673,852B2, entitled “Methods of treating neuronal disorders using MNTF peptides and analogs thereof”; U.S. Pat. No. 9,023,800B2, published May 5, 2015, entitled “Peptides for the treatment of oxidative stress related disorders”; US Patent Application Publication No. 20220154153A1, published May 19, 2022, entitled “New inhibitors of lrrk2/pp1 interaction”; the entire contents of each of which are herein incorporated by reference.

Certain polypeptides provided in this section may be useful in treating Parkinson's disease by modulating the activity of genes and gene products other than LRRK2 genes/gene products, such as SNCA genes/gene products.

### Small Molecules

LRRK2 expression and/or the activity of the protein it encodes in some embodiments can be modulated by the use of small molecules that can modulate LRRK2 (e.g., to modulate its biological activity, its expression, its localization, and/or its interaction with other biomolecules).

In some embodiments, examples of small molecules useful in the treatment of Parkinson's disease are provided in Jennings et al. “Preclinical and clinical evaluation of the LRRK2 inhibitor DNL201 for Parkinson's Disease” (2022) Sci Transl Med. 14(648) DOI: 10.1126/scitranslmed.abg7374; Azeggagh, et al. “The development of inhibitors of leucine-rich repeat kinase 2 (LRRK2) as a therapeutic strategy for Parkinson's disease: the current state of play” British Journal of Pharmacology 179(8): 1478-1495 (2021); U.S. Pat. No. 9,896,458B2, published Feb. 20, 2018, entitled “Compounds, compositions and methods”; U.S. Pat. No. 10,131,676B2, published Nov. 20, 2018, entitled “Compounds, compositions and methods”; U.S. Pat. No. 10,604,535B2, published Mar. 31, 2020, entitled “Compounds, compositions and methods”; U.S. Pat. No. 11,214,565B2, published Jan. 4, 2022, entitled “Compounds, compositions and methods”; U.S. Pat. No. 10,590,114B2, published Mar. 17, 2020, entitled “Compounds, compositions and methods”; U.S. Pat. No. 11,111,235B2, published Sep. 7, 2021, entitled “Compounds, compositions and methods”; U.S. Pat. No. 11,028,080B2, published Jun. 8, 2021, entitled “Substituted pyrimidines as LRKK2 inhibitors”; U.S. Pat. No. 9,156,845B2, published Oct. 13, 2015, entitled “4-(substituted amino)-7H-pyrrolo[2,3-d]pyrimidines as LRRK2 inhibitors”; U.S. Pat. No. 9,642,855B2, published May 9, 2017, entitled “Substituted pyrrolo[2,3-d]pyrimidines as LRRK2 inhibitors”; U.S. Pat. No. 10,039,753B2, published Aug. 7, 2018, entitled “Imidazo[4,5-c]quinoline and imidazo[4,5-c][1,5]naphthyridine derivatives as LRRK2 inhibitors”; U.S. Pat. No. 11,161,844B2, published Nov. 2, 2021, entitled “Cyclic substituted imidazo[4,5-c]quinoline derivatives”; U.S. Pat. No. 11,312,713B2, published Apr. 26, 2022, entitled “Imidazo[4,5-C]quinoline derivatives as LRRK2 inhibitors”; U.S. Pat. No. 9,499,535B2, published Nov. 22, 2016, entitled “Kinase inhibitors”; U.S. Pat. No. 9,802,937B2, published Oct. 31, 2017, entitled “Substituted pyrazolo{4,3-D}pyrimidines as kinase inhibitors”; U.S. Pat. No. 9,845,327B2, published Dec. 19, 2017, entitled “Treatment of proteinopathies”; U.S. Pat. No. 9,790,188B2, published Oct. 17, 2017, entitled “Benzimidazole derivatives and uses thereof”; U.S. Pat. No. 10,130,604B2, published Nov. 20, 2018, entitled “Composition and method for treating neurodegenerative disease”; US Patent Application Publication No. 20160250182A1, published Sep. 1, 2016, entitled “Rab7l1 interacts with lrrk2 to modify intraneuronal protein sorting and parkinson's disease risk”; US Patent Application Publication No. 20220048890A1, published Feb. 17, 2022, entitled “Methods and compositions for modulating splicing”; International PCT Application Publication No. WO2022093685A1, published May 5, 2022, entitled “Methods of treatment and diagnosis of parkinson's disease associated with wild-type lrrk2”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Parkinson's disease by modulating the activity of genes and gene products other than LRRK2 genes/gene products, such as SNCA genes/gene products.

### Gene Therapies

LRRK2 expression and/or activity in some embodiments can be modulated by the use of gene therapies that can modulate LRRK2 (e.g., by delivery of nucleic acids encoding LRRK2 or other molecules that interact with LRRK2 transcripts or the protein encoded by LRRK2).

In some embodiments, gene therapies, such as those involving administration of a compounds encoding useful therapeutic agents, useful in the treatment of Parkinson's disease are provided in U.S. Pat. No. 10,526,651B2, published Jan. 7, 2020, entitled “Modulators of alpha-synuclein toxicity”; U.S. Pat. No. 9,909,160B2, published Mar. 6, 2018, entitled “Modulators of alpha-synuclein toxicity”; US Patent Application Publication No. 20170035860A1, published Feb. 9, 2017, entitled “Compositions and methods for treatment of neurogenerative diseases”; International PCT Application Publication No. WO2019118727A2, published Jun. 20, 2019, entitled “Rescue of the pathology of lrrk2 on lysosmes with snx25 or snx27”; the entire contents of each of which are herein incorporated by reference.

Certain gene therapies provided in this section may be useful in treating Parkinson's disease by modulating the activity of genes and gene products other than LRRK2 genes/gene products, such as SNCA genes/gene products.

### Molecular Payloads Targeting SNCA

The SNCA gene, which encodes alpha-synuclein protein, and its aggregation has been implicated in Parkinson's disease, which affects nerves in the brain. Modulation of SNCA and/or alpha-synuclein expression and activity (e.g., by suppressing the expression of SNCA or mutant forms thereof, and/or activity of the encoded protein) therefore in some embodiments can have a therapeutic effect in subjects with Parkinson's disease.

### Oligonucleotides

SNCA expression and/or activity in some embodiments can be modulated by the use of oligonucleotides targeting SNCA sequences.

In some embodiments, an oligonucleotide useful for the treatment of Parkinson's disease, e.g., targeting (e.g., directly or indirectly modulating the expression or activity of) SNCA, comprises a region of complementarity to an SNCA transcript provided in Table 3, e.g., provided by any one of SEQ ID NOs: 417-434.

In some embodiments, examples of oligonucleotides useful for the treatment of Parkinson's disease, e.g., targeting (e.g., directly or indirectly modulating the expression or activity of) SNCA, are provided in Cole et al. (2018) “Alpha-synuclein antisense oligonucleotides as a disease-modifying therapy for Parkinson's disease” Mol Ther. 26(2):550-567; U.S. Pat. No. 9,290,759B2, published Mar. 22, 2016, entitled “Optimized miRNA constructs”; U.S. Pat. No. 10,724,035B2, published Jul. 28, 2020, entitled “Oligonucleotide compositions and methods thereof”; US Patent Application Publication No. 20040219671A1, published Nov. 4, 2004, entitled “RNA interference mediated treatment of parkinson disease using short interfering nucleic acid (siNA)”; U.S. Pat. No. 11,230,712B2, published Jan. 25, 2022, entitled “Compounds and methods for reducing SNCA expression”; U.S. Pat. No. 10,815,480B2, published Oct. 27, 2020, entitled “Modulation of alpha synuclein expression”; U.S. Pat. No. 9,663,783B2, published May 30, 2017, entitled “Modulation of alpha synuclein expression”; U.S. Pat. No. 7,595,306B2, published Sep. 29, 2009, entitled “Method of treating neurodegenerative disease”; US Patent Application Publication No. 20110257247A1, published Oct. 20, 2011, entitled “Method of Treating Neurodegenerative Disease”; U.S. Pat. No. 10,125,363B2, published Nov. 13, 2018, entitled “Compositions and methods for the treatment of parkinson disease by the selective delivery of oligonucleotide molecules to specific neuron types”; US Patent Application Publication No. 20200172903A1, published Jun. 4, 2020, entitled “ENA ANTISENSE OLIGONUCLEOTIDE FOR INHIBITION OF ALPHA-SYNUCLEIN EXPRESSION”; US Patent Application Publication No. 20200362347A1, published Nov. 19, 2020, entitled “Antisense oligonucleotides targeting alpha-synuclein and uses thereof”; U.S. Pat. No. 11,359,197B2, published Jun. 14, 2022, entitled “Antisense oligonucleotides targeting alpha-synuclein and uses thereof”; US Patent Application Publication No. 20220119811A1, published Apr. 21, 2022, entitled “Alpha-synuclein antisense oligonucleotides and uses thereof”; US Patent Application Publication No. 20210309999A1, published Oct. 7, 2021, entitled “VARIANT RNAi AGAINST ALPHA-SYNUCLEIN”; US Patent Application Publication No. 20210363524A1, published Nov. 25, 2021, entitled “Oligonucleotides for snca modulation”; International PCT Application Publication No. WO2022072447A1, published Apr. 7, 2022, entitled “Snca irna compositions and methods of use thereof for treating or preventing snca-associated neurodegenerative diseases”; the entire contents of each of which are herein incorporated by reference.

Certain oligonucleotides provided in this section may be useful in treating Parkinson's disease by modulating the activity of genes and/or gene products other than SNCA genes/gene products, such as LRRK2 genes/gene products.

### Polypeptides

SNCA expression and/or activity of the protein encoded thereby in some embodiments can be modulated by the use of polypeptides, such as polypeptides that can interact with SNCA nucleic acids and/or its encoded protein (e.g., to modulate its biological activity, its localization within the cell, its aggregation, and/or its interaction with other biomolecules).

In some embodiments, examples of polypeptides (e.g., peptides; proteins, such as enzymes; antibodies; etc.) useful in the treatment of Parkinson's disease are provided in U.S. Pat. No. 9,023,800B2, published May 5, 2015, entitled “Peptides for the treatment of oxidative stress related disorders”; U.S. Pat. No. 11,142,570B2, published Oct. 12, 2021, entitled “Antibodies to alpha-synuclein and uses thereof”; US Patent Application Publication No. 20180327471A1, published Nov. 15, 2018, entitled “Translatable molecules and synthesis thereof”; U.S. Pat. No. 10,066,007B2, published Sep. 4, 2019, entitled “Dipeptide-repeat proteins as therapeutic target in neurodegenerative diseases with hexanucleotide repeat expansion”; U.S. Pat. No. 8,673,852B2, entitled “Methods of treating neuronal disorders using MNTF peptides and analogs thereof”; U.S. Pat. No. 10,703,808B2, published Jul. 7, 2020, entitled “Human anti-alpha-synuclein antibodies”; International PCT Application Publication No. WO2018128454A1, entitled “ANTI-α-SYN ANTIBODY AND USE THEREOF”; U.S. Pat. No. 11,142,570B2, published Oct. 12, 2021, entitled “Antibodies to alpha-synuclein and uses thereof”; U.S. Pat. No. 11,220,538B2, published Jan. 11, 2022, entitled “Monoclonal antibodies against alpha-synuclein fibrils”; U.S. Pat. No. 11,155,608B2, published Oct. 26, 2021, entitled “Monoclonal antibodies against pathological alpha-synuclein, and methods using same”; U.S. Pat. No. 11,261,242B2, published Mar. 1, 2022, entitled “Anti-alpha-synuclein antibodies”; U.S. Pat. No. 11,292,831B2, published Apr. 5, 2022, entitled “Anti-alpha-synuclein antibodies”; US Patent Application Publication No. 20220033482A1, published Feb. 3, 2022, entitled “Alpha-synuclein single domain antibodies”; US Patent Application Publication No. 20210214429A1, published Jul. 15, 2021, entitled “Antibodies for the treatment of synucleinopathies and neuroinflammation”; US Patent Application Publication No. 20220194997A1, published Jun. 23, 2022, entitled “SUMO PEPTIDES FOR TREATING NEURODEGENERATIVE DISEASES”; International PCT Application Publication No. WO2021055881A1, published Mar. 25, 2021, entitled “Anti-alpha-synuclein antibodies and methods of use thereof”; International PCT Application Publication No. WO2021248038A1, published Dec. 9, 2021, entitled “Compositions and methods for the treatment of synucleinopathies”; the entire contents of each of which are herein incorporated by reference.

Certain polypeptides provided in this section may be useful in treating Parkinson's disease by modulating the activity of genes and/or gene products other than SNCA genes/gene products, such as LRRK2 genes/gene products.

### Small Molecules

SNCA expression and/or the activity of the protein it encodes in some embodiments can be modulated by the use of small molecules that can modulate SNCA (e.g., to modulate its biological activity, its expression, its localization, its aggregation, and/or its interaction with other biomolecules).

In some embodiments, examples of small molecules useful in the treatment of Parkinson's disease are provided in U.S. Pat. No. 9,790,188B2, published Oct. 17, 2017, entitled “Benzimidazole derivatives and uses thereof”; U.S. Pat. No. 8,232,402B2, published Jul. 31, 2012, entitled “Quinolinone farnesyl transferase inhibitors for the treatment of synucleinopathies and other indications”; U.S. Pat. No. 9,845,327B2, published Dec. 19, 2017, entitled “Treatment of proteinopathies”; U.S. Pat. No. 11,234,995B2, published Feb. 1, 2022, entitled “α-synuclein expression inhibitor”; U.S. Pat. No. 10,918,628B2, published Feb. 16, 2021, entitled “Treatment of synucleinopathies”; International PCT Application Publication No. WO2021127367A2, published Jun. 24, 2021, entitled “METHODS FOR INHIBITION OF ALPHA-SYNUCLEIN mRNA USING SMALL MOLECULES”; International PCT Application Publication No. WO2021141917A1, published Jul. 15, 2021, entitled “Antisense oligonucleotides for treatment of neurological disorders”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Parkinson's disease by modulating the activity of genes and gene products other than SNCA genes/gene products, such as LRRK2 genes/gene products.

### Gene Therapies

SNCA expression and/or activity in some embodiments can be modulated by the use of gene therapies that can modulate SNCA (e.g., by delivery of nucleic acids encoding SNCA or other molecules that interact with SNCA transcripts or the protein encoded by SNCA).

In some embodiments, gene therapies, such as those involving administration of a compounds encoding useful therapeutic agents, useful in the treatment of Parkinson's disease are provided in US Patent Application Publication No. 20170035860A1, published Feb. 9, 2017, entitled “Compositions and methods for treatment of neurogenerative diseases”; US Patent Application Publication No. 20180327471A1, published Nov. 15, 2018, entitled “Translatable molecules and synthesis thereof”; U.S. Pat. No. 10,526,651B2, published Jan. 7, 2020, entitled “Modulators of alpha-synuclein toxicity”; the entire contents of each of which are herein incorporated by reference.

Certain gene therapies provided in this section may be useful in treating Parkinson's disease by modulating the activity of genes and gene products other than SNCA genes/gene products, such as LRRK2 genes/gene products.

### Molecular Payloads for the Treatment of Essential Tremor

Various molecular payloads may be useful in the treatment of essential tremor, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of essential tremor may include, in some embodiments, molecular payloads which modulate (e.g., increase or decrease) expression or activity of a gene or genes expressed in the deep brain (e.g., the thalamus) and/or in the cerebellum.

### Molecular Payloads for the Treatment of Neuromuscular Diseases and Disorders

Various molecular payloads may be useful in the treatment of neuromuscular diseases and disorders, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of neuromuscular diseases and disorders may include, in some embodiments, molecular payloads which modulate (e.g., increase or decrease) expression or activity of DMPK, DMD, SMN, and/or FXN.

Examples of oligonucleotides useful for the treatment of neuromuscular diseases and disorders, e.g., oligonucleotides targeting (e.g., directly or indirectly modulating the expression or activity of) genes associated with neuromuscular diseases and disorders (e.g., DMPK, DMD, SMN, FXN, etc.), include those listed in Table 10 below. Each oligonucleotide provided in Table 10 may have any modification pattern disclosed herein.

TABLE 10
Oligonucleotides for the treatment of neuromuscular diseases and disorders

| | | Oligo- | |
|---|---|---|---|
| SEQ | | nucleotide | |
| ID | Target | Sequence | |
| NO: | gene | (5′-3′) | Oligonucleotide Structure (5′-3′) |
| 87 | DMPK | ACAATAAATA | Aks mCks Aks Ads Tds Ads Ads Ads Tds Ads mCds |
| | | CCGAGG | mCds Gds Aks Gks Gk |
| 88 | DMPK | CGGAGCGGTT | mCes Ges Ges Aes Ges mCds Gds Gds Tds Tds Gds |
| | | GTGAACTGGC | Tds Gds Ads Ads mCes Tes Ges Ges mCe |
| 89 | DMPK | GCGCACCTTC | Ges mCes Ges mCes Aes mCds mCds Tds Tds mCds |
| | | CCGAATGTCC | mCds mCds Gds Ads Ads Tes Ges Tes mCes mCe |
| 90 | DMPK | CCCGAATGTC | mCes mCes mCks Gks Ads Ads Tds Gds Tds mCds |
| | | CGACAG | mCds Gds Aks mCks Aes Ge |
| 91 | DMPK | CGGAGCGGTT | mCes Ges Ges Aks Gks mCds Gds Gds Tds Tds Gds |
| | | GTGAACT | Tds Gks Aks Aes mCes Te |
| 92 | DMPK | ACAATAAATA | Aes mCes Aes Aks Tks Ads Ads Ads Tds Ads mCds |
| | | CCGAGGA | mCds Gks Aks Ges Ges Ae |
| 93 | DMPK | GACAATCTCC | Gks Aks mCks Ads Ads Tds mCds Tds mCds mCds |
| | | GCCAGG | Gds mCds mCds Aks Gks Gk |
| 94 | DMPK | TCCCGAATGT | Tes mCes mCks mCks Gds Ads Ads Tds Gds Tds |
| | | CCGACA | mCds mCds Gks Aks mCes Ae |
| 95 | DMPK | TGTAATGTTG | Tks Gks Tks Ads Ads Tds Gds Tds Tds Gds Tds |
| | | TCCAGT | mCds mCds Aks Gks Tk |
| 96 | DMPK | GTGTAATGTT | Ges Tes Gks Tks Ads Ads Tds Gds Tds Tds Gds |
| | | GTCCAG | Tds mCks mCks Aes Ge |
| 97 | DMD | UCAAGGAAGA | fU*SfC*SfA*SfA*SfG*SfG*SmAfA*SmGmA*SfU*SmGmGf |
| | | UGGCAUUUCU | C*SfA*SfU*SfU*SfU*SfC*SfU, wherein: f = 2′-F |
| | | | modification to a nucleoside; S = Sp phosphorothioate; |
| | | | m = 2′-OMe modification to a nucleoside |
| 98 | DMD | UCAAGGAAGA | fU*SfC*SfA*SfA*SfG*SfG*SmAfA*SmGmA*SfU*SmGmGf |
| | | UGGCAUUUCU | C*SfA*Sf*SfU*SfU*SfU*SfC*SfU, wherein: f = 2′-F |
| | | | modification to a nucleoside; S = Sp phosphorothioate; |
| | | | m = 2′-OMe modification to a nucleoside |
| 99 | FXN | CUUCUTCTTC | oC*oU*oU*oC*oU*dT*xdC*dT*dT*xdC*dT*dT*xdC*dT* |
| | | TTCTTCUUCU | dT*oC*oU*oU*oC*oU |
| 100 | FXN | UUCUUCTTCT | oU*oU*oC*oU*oU*xdC*dT*dT*xdC*dT*dT*xdC*dT*dT* |
| | | TCTTCUUCUU | xdC*oU*oU*oC*oU*oU |
| 101 | FXN | UCUUCTTCTT | oU*oC*oU*oU*oC*dT*dT*xdC*dT*dT*xdC*dT*dT*xdC* |
| | | CTTCTUCUUC | dT*oU*oC*oU*oU*oC |
| 102 | FXN | CUUCUUCUUC | oC*oU*oU*oC*oU*oU*oC*oU*oU*oC*oU*oU*oC*oU*oU* |
| | | UUCUUCUUCU | oC*oU*oU*oC*oU |
| 103 | FXN | UUCUUCUUCU | oU*oU*oC*oU*oU*oC*oU*oU*oC*oU*oU*oC*oU*oU*oC* |
| | | UCUUCUUCUU | oU*oU*oC*oU*oU |
| 104 | FXN | UCUUCUUCUU | oU*oC*oU*oU*oC*oU*oU*oC*oU*oU*oC*oU*oU*oC*oU* |
| | | CUUCUUCUUC | oU*oC*oU*oU*oC |
| 105 | FXN | CUUCTTCTTC | +C*+U*+U*xdC*dT*dT*xdC*dT*dT*xdC*dT*dT*xdC*dT |
| | | TTCTTCTUCU | *dT*xdC*dT*+U*+C*+U |
| 106 | FXN | UUCTTCTTCT | +U*+U*+C*dT*dT*xdC*dT*dT*xdC*dT*dT*xdC*dT*dT* |
| | | TCTTCTTCUU | xdC*dT*dT*+C*+U*+U |
| 107 | FXN | UCUTCTTCTT | +U*+C*+U*dT*xdC*dT*dT*xdC*dT*dT*xdC*dT*dT*xdC |
| | | CTTCTTCUUC | *dT*dT*xdC*+U*+U*+C |
| 108 | FXN | CUUCUTCTTC | +C*+U*+U*+C*+U*dT*xdC*dT*dT*xdC*dT*dT*xdC*dT* |
| | | TTCTTCUUCU | dT*+C*+U*+U*+C*+U |
| 109 | FXN | UUCUUCTTCT | +U*+U*+C*+U*+U*xdC*dT*dT*xdC*dT*dT*xdC*dT*dT* |
| | | TCTTCUUCUU | xdC*+U*+U*+C*+U*+U |
| 110 | FXN | UCUUCTTCTT | +U*+C*+U*+U*+C*dT*dT*xdC*dT*dT*xdC*dT*dT*xdC* |
| | | CTTCTUCUUC | dT*+U*+C*+U*+U*+C |
| 111 | FXN | CUUCUUCUUC | xdC*+U*+U*xdC*+U*+U*xdC*+U*+U*xdC*+U*+U*xdC*+ |
| | | UUCUUCUUCU | U*+U*xdC*+U*+U*xdC*+U |
| 112 | FXN | UUCUUCUUCU | +U*+U*xdC*+U*+U*xdC*+U*+U*xdC*+U*+U*xdC*+U*+U |
| | | UCUUCUUCUU | *xdC*+U*+U*xdC*+U*+U |
| 113 | FXN | UCUUCUUCUU | +U*xdC*+U*+U*xdC*+U*+U*xdC*+U*+U*xdC*+U*+U*xd |
| | | CUUCUUCUUC | C*+U*+U*xdC*+U*+U*xdC |
| 114 | FXN | CTTCTTCTTC | +C*dT*dT*+C*dT*dT*+C*dT*dT*+C*dT*dT*+C*dT*dT* |
| | | TTCTTCTTCT | +C*dT*dT*+C*dT |
| 115 | FXN | TTCTTCTTCT | dT*dT*+C*dT*dT*+C*dT*dT*+C*dT*dT*+C*dT*dT*+C* |
| | | TCTTCTTCTT | dT*dT*+C*dT*dT |
| 116 | FXN | TCTTCTTCTT | dT*+C*dT*dT*+C*dT*dT*+C*dT*dT*+C*dT*dT*+C*dT* |
| | | CTTCTTCTTC | dT*+C*dT*dT*+C |
| 117 | SMN | GTCTGCCAGC | Gns Tnx mCnx Tnx Gnx mCnx mCnx Anx Gnx mCnx |
| | | ATTATGAAAG | Anx Tnx Tnx Anx Tnx Gnx Anx Anx Ans Gn |
| 118 | SMN | TCTGCCAGCA | Tns mCnx Tnx Gnx mCnx mCnx Anx Gnx mCnx Anx |
| | | TTATGAAAGT | Tnx Tnx Anx Tnx Gnx Anx Anx Anx Gns Tn |
| 119 | SMN | TCTGCCAGCA | Tns mCnx Tnx Gnx mCnx mCnx Anx Gnx mCnx Anx |
| | | TTATGAAAG | Tnx Tnx Anx Tnx Gnx Anx Anx Ans Gn |
| 120 | SMN | CTGCCAGCAT | mCns Tnx Gnx mCnx mCnx Anx Gnx mCnx Anx Tnx |
| | | TATGAAAGTG | Tnx Anx Tnx Gnx Anx Anx Anx Gnx Tns Gn |
| 121 | SMN | CTGCCAGCAT | mCns Tnx Gnx mCnx mCnx Anx Gnx mCnx Anx Tnx |
| | | TATGAAAGT | Tnx Anx Tnx Gnx Anx Anx Anx Gns Tn |
| 122 | SMN | CTGCCAGCAT | mCns Tnx Gnx mCnx mCnx Anx Gnx mCnx Anx Tnx |
| | | TATGAAAG | Tnx Anx Tnx Gnx Anx Anx Ans Gn |
| 123 | SMN | TGCCAGCATT | Tns Gnx mCnx mCnx Anx Gnx mCnx Anx Tnx Tnx |
| | | ATGAAAGTGA | Anx Tnx Gnx Anx Anx Anx Gnx Tnx Gns An |
| 124 | SMN | TGCCAGCATT | Tns Gnx mCnx mCnx Anx Gnx mCnx Anx Tnx Tnx |
| | | ATGAAAGTG | Anx Tnx Gnx Anx Anx Anx Gnx Tns Gn |
| 125 | SMN | TGCCAGCATT | Tns Gnx mCnx mCnx Anx Gnx mCnx Anx Tnx Tnx |
| | | ATGAAAGT | Anx Tnx Gnx Anx Anx Anx Gns Tn |
| 126 | SMN | TGCCAGCATT | Tns Gnx mCnx mCnx Anx Gnx mCnx Anx Tnx Tnx |
| | | ATGAAAG | Anx Tnx Gnx Anx Anx Ans Gn |
| 127 | SMN | GCCAGCATTA | Gns mCnx mCnx Anx Gnx mCnx Anx Tnx Tnx Anx |
| | | TGAAAGTGAA | Tnx Gnx Anx Anx Anx Gnx Tnx Gnx Ans An |
| 128 | SMN | GCCAGCATTA | Gns mCnx mCnx Anx Gnx mCnx Anx Tnx Tnx Anx |
| | | TGAAAGTGA | Tnx Gnx Anx Anx Anx Gnx Tnx Gns An |
| 129 | SMN | GCCAGCATTA | Gns mCnx mCnx Anx Gnx mCnx Anx Tnx Tnx Anx |
| | | TGAAAGTG | Tnx Gnx Anx Anx Anx Gnx Tns Gn |
| 130 | SMN | GCCAGCATTA | Gns mCnx mCnx Anx Gnx mCnx Anx Tnx Tnx Anx |
| | | TGAAAGT | Tnx Gnx Anx Anx Anx Gns Tn |
| 131 | SMN | GCCAGCATTA | Gns mCnx mCnx Anx Gnx mCnx Anx Tnx Tnx Anx |
| | | TGAAAG | Tnx Gnx Anx Anx Ans Gn |
| 132 | SMN | CCAGCATTAT | mCns mCnx Anx Gnx mCnx Anx Tnx Tnx Anx Tnx |
| | | GAAAGTGAAT | Gnx Anx Anx Anx Gnx Tnx Gnx Anx Ans Tn |
| 133 | SMN | CCAGCATTAT | mCns mCnx Anx Gnx mCnx Anx Tnx Tnx Anx Tnx |
| | | GAAAGTGA | Gnx Anx Anx Anx Gnx Tnx Gns An |
| 134 | SMN | CCAGCATTAT | mCns mCnx Anx Gnx mCnx Anx Tnx Tnx Anx Tnx |
| | | GAAAGTG | Gnx Anx Anx Anx Gnx Tns Gn |
| 135 | SMN | CCAGCATTAT | mCns mCnx Anx Gnx mCnx Anx Tnx Tnx Anx Tnx |
| | | GAAAGT | Gnx Anx Anx Anx Gns Tn |
| 136 | SMN | CCAGCATTAT | mCns mCnx Anx Gnx mCnx Anx Tnx Tnx Anx Tnx |
| | | GAAAG | Gnx Anx Anx Anx Gn |
| 137 | SMN | CAGCATTATG | mCns Anx Gnx mCnx Anx Tnx Tnx Anx Tnx Gnx Anx |
| | | AAAGTGAATC | Anx Anx Gnx Tnx Gnx Anx Anx Tns mCn |
| 138 | SMN | CAGCATTATG | mCns Anx Gnx mCnx Anx Tnx Tnx Anx Tnx Gnx Anx |
| | | AAAGTGAAT | Anx Anx Gnx Tnx Gnx Anx Ans Tn |
| 139 | SMN | CAGCATTATG | mCns Anx Gnx mCnx Anx Tnx Tnx Anx Tnx Gnx Anx |
| | | AAAGTGA | Anx Anx Gnx Tnx Gns An |
| 140 | SMN | CAGCATTATG | mCns Anx Gnx mCnx Anx Tnx Tnx Anx Tnx Gnx Anx |
| | | AAAGTG | Anx Anx Gnx Tns Gn |
| 141 | SMN | CAGCATTATG | mCns Anx Gnx mCnx Anx Tnx Tnx Anx Tnx Gnx Anx |
| | | AAAGT | Anx Anx Gns Tn |
| 1069 | SMN | UCACUUUCAU | mUes mCes Aes mCes mUes mUes mUes mCes Aes |
| | | AAUGCUGG | mUes Aes Aes mUes Ges mCes mUes Ges Ges |
| | | | (Nusinersen) |

Abbreviations used in Table 10:
mC = 5-methyl cytosine; n = 2′-O-(N-methylacetamide) (2′-NMA) sugar moiety; s = phosphorothioate internucleoside linkage; o = phosphodiester internucleoside linkage; e = 2′-MOE modified sugar; k = cEt modified sugar. FXN-targeting oligonucleotides: “xdC” indicates a 5-methyl-deoxycytidine; “dN” indicates a 2′-deoxyribonucleoside; “+N” indicates a LNA nucleoside; “ON” indicates a 2′- MOE modified ribonucleoside; “oC” indicates a 5-methyl-2′-MOE-cytidine; “+C” indicates a 5-methyl-2′-4′-bicyclic-cytidine (2′-4′ methylene bridge); “OU” indicates a 5-methyl-2′-MOE-uridine; “+U” indicates a 5-methyl-2′-4′-bicyclic-uridine (2′-4′ methylene bridge); “*” indicates a phosphorothioate internucleoside linkage. SMN-targeting oligonucleotides: mC = 5-methyl cytosine; mU = 5-methyl uridine; e = 2′-MOE modified sugar; n = 2′-NMA sugar moiety; x = internucleoside linkage that is either phosphorothioate or phosphodiester; s = phosphorothioate internucleoside linkage. In each sequence listed in Table 10, each T may be optionally and independently replaced with a U, and each U may be optionally and independently replaced with a T.

Additional examples of oligonucleotides useful for the treatment of neuromuscular diseases and disorders, e.g., oligonucleotides targeting (e.g., directly or indirectly modulating the expression or activity of) genes associated with neuromuscular diseases and disorders (e.g., SMN), include those listed in Table 11 below. Each oligonucleotide provided in Table 11 may have any modification pattern disclosed herein.

TABLE 11
Oligonucleotides for the treatment of neuromuscular diseases and disorders

| | | Oligo-- | |
|---|---|---|---|
| SEQ | | nucleotide | |
| ID | Target | Sequence | |
| NO: | gene | (5′-3′) | Oligonucleotide Structure (5′-3′) |
| 1016 | SMN | CACTTTCATA | mns no mns no ns ns mns ns ns ns ns ns ns mns |
| | | ATGCTGGC | ns ns ns mn, wherein: m = 5-methyl nucleobase, |
| | | | n = 2′-NMA sugar moiety, s = phosphorothioate |
| | | | internucleoside linkage, and o = phosphodiester |
| | | | internucleoside linkage |
| 1016 | SMN | CACTTTCATA | mes eo mes eo es es mes es es es es es es mes |
| | | ATGCTGGC | es es es me, wherein: m = 5-methyl nucleobase, |
| | | | e = 2′-MOE sugar moiety, s = phosphorothioate |
| | | | internucleoside linkage, and o = phosphodiester |
| | | | internucleoside linkage |
| 1017 | SMN | TTCACTTTCA | eo es mes es mes es es es mes es es es es es |
| | | TAATGCTGGC | es mes es es eo me, wherein: m = 5-methyl |
| | | | nucleobase, e = 2′-MOE sugar moiety, |
| | | | s = phosphorothioate internucleoside linkage, |
| | | | and o = phosphodiester internucleoside linkage |
| 1017 | SMN | TTCACTTTCA | eo os mns os mns os os os mns os os os os os |
| | | TAATGCTGGC | os mns os os no me, wherein: m = 5-methyl |
| | | | nucleobase, n = 2′-NMA sugar moiety, e = 2′-MOE |
| | | | sugar moiety, s = phosphorothioate |
| | | | internucleoside linkage, and o = phosphodiester |
| | | | internucleoside linkage |

In each sequence listed in Table 11, each T may be optionally and independently replaced with a U.

Examples of small molecules useful for the treatment of neuromuscular diseases and disorders include

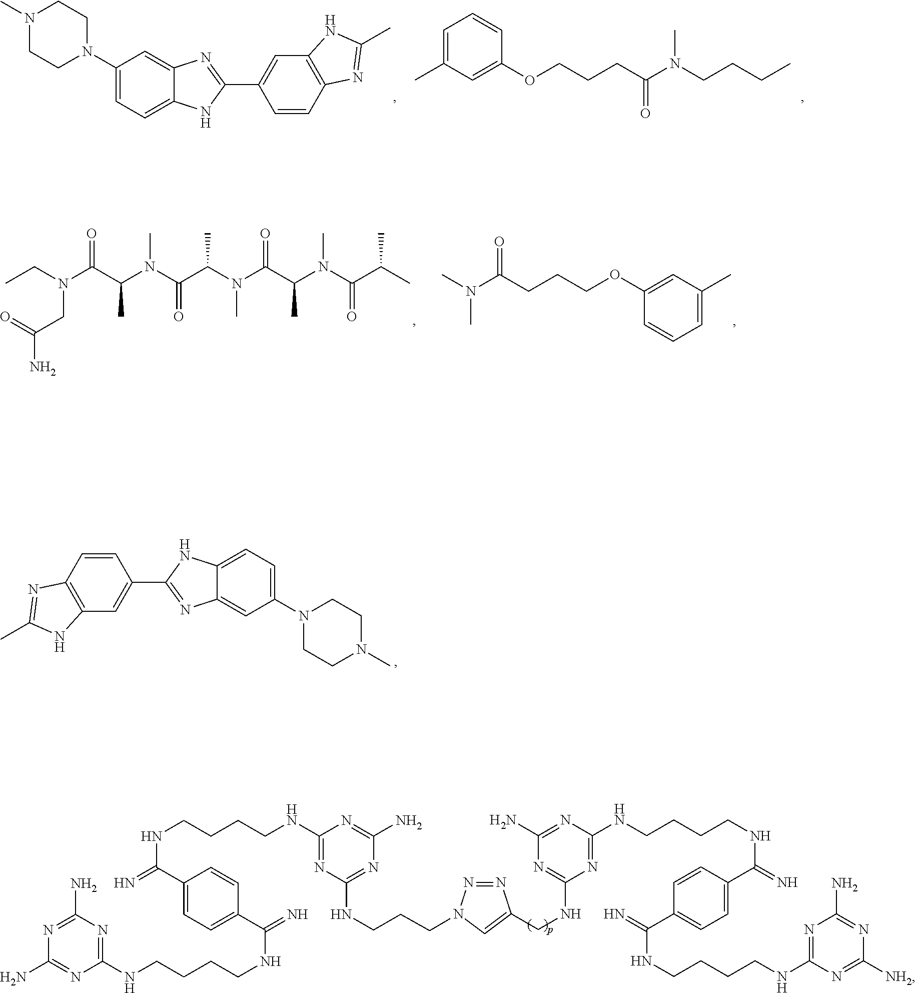

wherein p is 1 or 2,

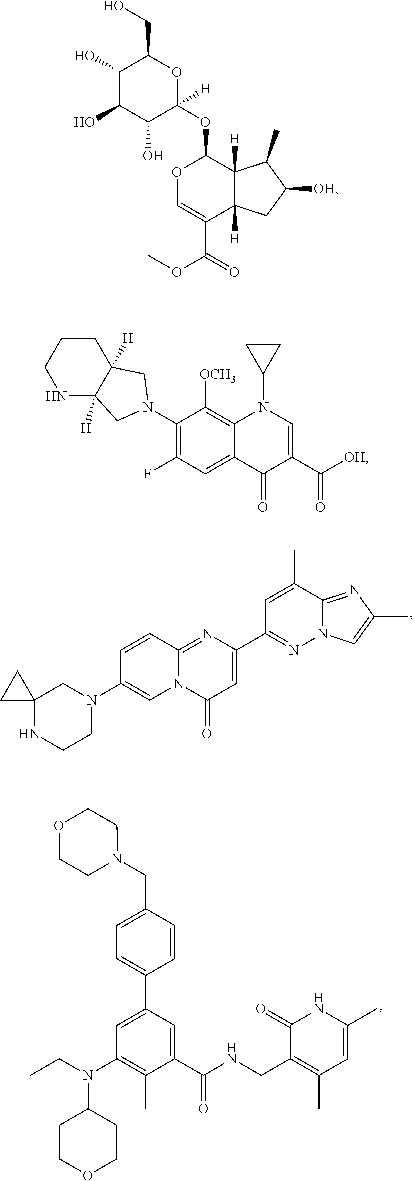

and pharmaceutically acceptable salts, co-crystals, tautomers, stereoisomers, solvates, hydrates, polymorphs, isotopically enriched compounds, and prodrugs thereof.

Examples of polypeptides useful for the treatment of neuromuscular diseases and disorders include antibodies, proteins, peptides, and enzymes. In some embodiments, an antibody useful for the treatment of neuromuscular diseases is apitegromab.

Examples of gene therapies useful for the treatment of neuromuscular disease and disorders include Onasemnogene abeparvovec (and biosimilars thereof).

### Molecular Payloads Targeting DMPK

The DMPK gene, which encodes myotonic dystrophy protein kinase, and mutations therein are implicated in myotonic dystrophy, a neuromuscular disease. Modulation of DMPK expression and activity (e.g., by suppressing the expression of mutant DMPK and/or activity of the myotonic dystrophy protein kinase protein) therefore in some embodiments can have a therapeutic effect in subjects with myotonic dystrophy.

### Oligonucleotides

DMPK expression and/or activity in some embodiments can be modulated by the use of oligonucleotides targeting DMPK sequences.

In some embodiments, an oligonucleotide useful for the treatment of myotonic dystrophy, e.g., targeting (e.g., directly or indirectly modulating the expression or activity of) DMPK, comprises a region of complementarity to a DMPK transcript provided in Table 3, e.g., provided by any one of SEQ ID NOs: 438-444.

In some embodiments, examples of oligonucleotides useful for the treatment of treatment of neuromuscular diseases and disorders (e.g., myotonic dystrophy), e.g., targeting (e.g., directly or indirectly modulating the expression or activity of) DMPK, are provided in US Patent Application Publication No. 20140378533A1, published Dec. 25, 2014, entitled “Modulation of RNA by repeat targeting”; US Patent Application Publication No. 20150148404A1, published May 28, 2015, entitled “RNA Modulating Oligonucleotides with Improved Characteristics for the Treatment of Neuromuscular Disorders”; U.S. Pat. No. 11,345,915B2, published May 31, 2022, entitled “RNA modulating oligonucleotides with improved characteristics for the treatment of neuromuscular disorders”; US Patent Application Publication No. 20210269825A1, published Sep. 2, 2021, entitled “Compositions and methods for reducing spliceopathy and treating ma dominance disorders”; International PCT Application Publication No. WO2021028666A1, published Feb. 18, 2021, entitled “Conjugate and uses thereof”; US Patent Application Publication No. 20130059902A1, published Mar. 7, 2013, entitled “Methods and compositions useful in treatment of diseases or conditions related to repeat expansion”; U.S. Pat. No. 10,526,604B2, published Jan. 7, 2020, entitled “Modulation of nuclear-retained RNA”; U.S. Pat. No. 10,238,753B2, published Mar. 26, 2019, entitled “Antisense conjugates for decreasing expression of DMPK”; U.S. Pat. No. 10,111,962B2, published Oct. 30, 2018, entitled “Peptide-linked morpholino antisense oligonucleotides for treatment of myotonic dystrophy”; U.S. Pat. No. 10,954,519B2, published Mar. 23, 2021, entitled “Compounds and methods for modulation of dystrophia myotonica-protein kinase (DMPK) expression”; US Patent Application Publication No. 20210315918A1, published Oct. 14, 2021, entitled “Compounds and Methods for Modulation of Transcript Processing”; U.S. Pat. No. 9,765,338B2, published Sep. 19, 2017, entitled “Modulation of dystrophia myotonica-protein kinase (DMPK) expression”; the entire contents of each of which are herein incorporated by reference.

Certain oligonucleotides provided in this section may be useful in treating neuromuscular disease or disorders by modulating the activity of genes and/or gene products other than DMPK genes/gene products, such as DMD, SMN, and/or FXN genes/gene products.

### Polypeptides

DMPK expression and/or activity of the protein encoded thereby in some embodiments can be modulated by the use of polypeptides, such as polypeptides that can interact with DMPK nucleic acids and/or its encoded protein (e.g., to modulate its biological activity, its localization within the cell, and/or its interaction with other biomolecules).

In some embodiments, examples of polypeptides (e.g., peptides; proteins, such as enzymes; antibodies; etc.) useful in the treatment of neuromuscular diseases and disorders (e.g., myotonic dystrophy) are provided in International PCT Application Publication No. WO2021028666A1, published Feb. 18, 2021, entitled “Conjugate and uses thereof”; U.S. Pat. No. 9,114,178B2, published Aug. 25, 2015, entitled “Methods and compositions for treatment of myotonic dystrophy”; U.S. Pat. No. 10,799,556B2, published Oct. 13, 2020, entitled “Treatment of myotonic dystrophy”; the entire contents of each of which are herein incorporated by reference.

Certain polypeptides provided in this section may be useful in treating neuromuscular diseases or disorders by modulating the activity of genes and/or gene products other than DMPK genes/gene products, such as DMD, SMN, and/or FXN genes/gene products.

### Small Molecules

DMPK expression and/or the activity of the protein it encodes in some embodiments can be modulated by the use of small molecules that can modulate DMPK (e.g., to modulate its biological activity, its expression, its localization, and/or its interaction with other biomolecules).

In some embodiments, examples of small molecules useful in the treatment of neuromuscular diseases and disorders (e.g., myotonic dystrophy) are provided in US Patent Application Publication No. 20140187595A1, published Jul. 3, 2014, entitled “Methods and Compositions Comprising AMPK Activator (Metformin/Troglitazone) for the Treatment of Myotonic Dystrophy Type 1 (DM1)”; U.S. Pat. No. 11,103,514B2, published Aug. 31, 2021, entitled “Treatment of muscular dystrophy”; US Patent Application Publication No. 20140121236A1, published May 1, 2014, entitled “Compositions and Methods for Treating Myotonic Dystrophy Type 1”; U.S. Pat. No. 9,933,419B2, published Apr. 3, 2018, entitled “Specific targeting of RNA expanded repeat sequences”; U.S. Pat. No. 9,795,687B2, published Oct. 24, 2017, entitled “Modularly assembled small molecules for the treatment of myotonic dystrophy type 1”; U.S. Pat. No. 10,266,520B2, published Apr. 23, 2019, entitled “Bisamidinium-based inhibitors for the treatment of myotonic dystrophy”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating neuromuscular diseases or disorders by modulating the activity of genes and/or gene products other than DMPK genes/gene products, such as DMD, SMN, and/or FXN genes/gene products.

### Gene Therapies

DMPK expression and/or activity in some embodiments can be modulated by the use of gene therapies that can modulate DMPK (e.g., by delivery of nucleic acids encoding DMPK or other molecules that interact with DMPK transcripts or the protein encoded by DMPK).

In some embodiments, gene therapies, such as those involving administration of compounds encoding useful therapeutic agents, useful in the treatment of neuromuscular diseases and disorders (e.g., myotonic dystrophy) are provided in US Patent Application Publication No. 20180327471A1, published Nov. 15, 2018, entitled “Translatable molecules and synthesis thereof”; US Patent Application Publication No. 20210269825A1, published Sep. 2, 2021, entitled “Compositions and methods for reducing spliceopathy and treating rna dominance disorders”; the entire contents of each of which are herein incorporated by reference.

Certain gene therapies provided in this section may be useful in treating neuromuscular diseases or disorders by modulating the activity of genes and/or gene products other than DMPK genes/gene products, such as DMD, SMN, and/or FXN genes/gene products.

### Molecular Payloads Targeting DMD

The DMD gene, which encodes dystrophin protein, is associated with Duchenne muscular dystrophy and neurological disorders. Modulation of DMD and/or dystrophin expression and activity (e.g., by suppressing the expression of DMD or mutant forms thereof, and/or activity of the encoded protein) therefore in some embodiments can have a therapeutic effect in subjects with Duchenne muscular dystrophy and neurological disorders associated with DMD.

### Oligonucleotides

DMD expression and/or activity in some embodiments can be modulated by the use of oligonucleotides targeting DMD sequences.

In some embodiments, an oligonucleotide useful for the treatment of Duchenne muscular dystrophy and neurological disorders associated with DMD, e.g., targeting (e.g., directly or indirectly modulating the expression or activity of) DMD, comprises a region of complementarity to a DMD transcript provided in Table 3, e.g., provided by any one of SEQ ID NOs: 445-474.

In some embodiments, examples of oligonucleotides useful for the treatment of Duchenne muscular dystrophy and neurological disorders associated with DMD, e.g., targeting (e.g., directly or indirectly modulating the expression or activity of) DMD, are provided in U.S. Pat. No. 10,995,337B2, published May 4, 2021, entitled “Antisense oligonucleotides for inducing exon skipping and methods of use thereof”; US Patent Application Publication No. 20110046200A1, published Feb. 24, 2011, entitled “Use of antisense oligonucleotides to effect translation modulation”; U.S. Pat. No. 10,876,114B2, published Dec. 29, 2020, entitled “Methods and means for efficient skipping of at least one of the following exons of the human Duchenne muscular dystrophy gene: 43, 46, 50-53”; U.S. Pat. No. 10,246,707B2, published Apr. 2, 2019, entitled “Method for efficient exon (44) skipping in duchenne muscular dystrophy and associated means”; U.S. Pat. No. 9,447,417B2, published Sep. 20, 2016, entitled “Multiple exon skipping compositions for DMD”; U.S. Pat. No. 10,822,400B2, published Nov. 3, 2020, entitled “Dynamin 2 inhibitor for the treatment of Duchenne's muscular dystrophy”; U.S. Pat. No. 10,457,944B2, published Oct. 19, 2019, entitled “Oligomers”; U.S. Pat. No. 11,034,956B2, published Jun. 15, 2021, entitled “Oligonucleotide comprising an inosine for treating DMD”; U.S. Pat. No. 9,533,004B2, published Jan. 3, 2017, entitled “Treatment of dystrophin family related diseases by inhibition of natural antisense transcript to DMD family”; U.S. Pat. No. 10,913,946 BB, published Feb. 9, 2021, entitled “RNA modulating oligonucleotides with improved characteristics for the treatment of Duchenne and Becker muscular dystrophy”; U.S. Pat. No. 10,450,568B2, published Oct. 22, 2019, entitled “Oligonucleotide compositions and methods thereof”; US Patent Application Publication No. 20210315918A1, published Oct. 14, 2021, entitled “Compounds and Methods for Modulation of Transcript Processing”; U.S. Pat. No. 11,053,497B2, published Jul. 6, 2021, entitled “Antisense nucleic acids”; U.S. Pat. No. 10,752,898B2, published Aug. 25, 2020, entitled “Effective gene therapy tools for dystrophin exon 53 skipping”; U.S. Pat. No. 11,236,336B2, published Feb. 1, 2022, entitled “Therapeutic targeting of a microRNA to treat Duchenne muscular dystrophy”; the entire contents of each of which are herein incorporated by reference.

Certain oligonucleotides provided in this section may be useful in treating Duchenne muscular dystrophy and neurological disorders associated with DMD by modulating the activity of genes and/or gene products other than DMD genes/gene products, such as DMPK, SMN, and/or FXN genes/gene products.

### Polypeptides

DMD expression and/or activity of the protein encoded thereby in some embodiments can be modulated by the use of polypeptides, such as polypeptides that can interact with DMD nucleic acids and/or its encoded protein (e.g., to modulate its biological activity, its localization within the cell, its aggregation, and/or its interaction with other biomolecules).

In some embodiments, examples of polypeptides (e.g., peptides; proteins, such as enzymes; antibodies; etc.) useful in the treatment of Duchenne muscular dystrophy and neurological disorders associated with DMD are provided in U.S. Pat. No. 11,339,209B2, published May 24, 2022, entitled “Compositions, methods, and therapeutic uses related to fusogenic protein minion”; International PCT Application Publication No. WO2021089736A1, published May 14, 2021, entitled “Combined therapy for muscular diseases”; the entire contents of each of which are herein incorporated by reference.

Certain polypeptides provided in this section may be useful in treating Duchenne muscular dystrophy and neurological disorders associated with DMD by modulating the activity of genes and/or gene products other than DMD genes/gene products, such as DMPK, SMN, and/or FXN genes/gene products.

### Small Molecules

DMD expression and/or the activity of the protein it encodes in some embodiments can be modulated by the use of small molecules that can modulate DMD (e.g., to modulate its biological activity, its expression, its localization, its aggregation, and/or its interaction with other biomolecules).

In some embodiments, examples of small molecules useful in the treatment of Duchenne muscular dystrophy and neurological disorders associated with DMD are provided in US Patent Application Publication No. 20130210753A1, published Aug. 15, 2013, entitled “Methods of treating muscular dystrophies”; US Patent Application Publication No. 20160207893A1, published Jul. 21, 2016, entitled “Novel calcium modulators”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Duchenne muscular dystrophy and neurological disorders associated with DMD by modulating the activity of genes and/or gene products other than DMD genes/gene products, such as DMPK, SMN, and/or FXN genes/gene products.

### Gene Therapies

DMD expression and/or activity in some embodiments can be modulated by the use of gene therapies that can modulate DMD (e.g., by delivery of nucleic acids encoding DMD or other molecules that interact with DMD transcripts or the protein encoded by DMD).

In some embodiments, gene therapies, such as those involving administration of a compounds encoding useful therapeutic agents, useful in the treatment of Duchenne muscular dystrophy and neurological disorders associated with DMD are provided in U.S. Pat. No. 10,301,367B2, published May 28, 2019, entitled “Compositions and methods for treatment of muscular dystrophy”; U.S. Pat. No. 10,815,463B2, published Oct. 27, 2020, entitled “Messenger UNA molecules and uses thereof”; U.S. Pat. No. 10,647,751B2, published May 12, 2020, entitled “Production of large-sized microdystrophins in an AAV-based vector configuration”; U.S. Pat. No. 10,752,898B2, published Aug. 25, 2020, entitled “Effective gene therapy tools for dystrophin exon 53 skipping”; U.S. Pat. No. 11,339,209B2, published May 24, 2022, entitled “Compositions, methods, and therapeutic uses related to fusogenic protein minion”; US Patent Application Publication No. 20180327471A1, published Nov. 15, 2018, entitled “Translatable molecules and synthesis thereof”; International PCT Application Publication No. WO2021089736A1, published May 14, 2021, entitled “Combined therapy for muscular diseases”; the entire contents of each of which are herein incorporated by reference.

Certain gene therapies provided in this section may be useful in treating Duchenne muscular dystrophy and neurological disorders associated with DMD by modulating the activity of genes and/or gene products other than DMD genes/gene products, such as DMPK, SMN, and/or FXN genes/gene products.

### Molecular Payloads Targeting FXN

The FXN gene, which encodes frataxin protein, is associated with Friedreich's ataxia, a neurological/neuromuscular disorder. Modulation of FXN and/or frataxin expression and activity (e.g., by suppressing the expression of FXN or mutant forms thereof, and/or activity of the encoded protein) therefore in some embodiments can have a therapeutic effect in subjects with Friedreich's ataxia and neurological disorders associated with FXN.

### Oligonucleotides

FXN expression and/or activity in some embodiments can be modulated by the use of oligonucleotides targeting FXN sequences.

In some embodiments, an oligonucleotide useful for the treatment of Friedreich's ataxia and neurological disorders associated with FXN, e.g., targeting (e.g., directly or indirectly modulating the expression or activity of) FXN, comprises a region of complementarity to an FXN transcript provided in Table 3, e.g., provided by any one of SEQ ID NOs: 497-498.

In some embodiments, examples of oligonucleotides useful for the treatment of Friedreich's ataxia and neurological disorders associated with FXN, e.g., targeting (e.g., directly or indirectly modulating the expression or activity of) FXN, are provided in Li, L. et al “Activating frataxin expression by repeat-targeted nucleic acids” Nat. Comm. 2016, 7:10606.; Li L. et al. “Activation of Frataxin Protein Expression by Antisense Oligonucleotides Targeting the Mutant Expanded Repeat” Nucleic Acid Ther. 2018 February; 28(1):23-33.; US Patent Application Publication No. 20220017903A1, published Jan. 20, 2022, entitled “Compositions and methods for treatment of Friedreich's ataxia”; US Patent Application Publication No. 20210308274A1, published Oct. 7, 2021, entitled “Muscle targeting complexes and uses thereof for treating Friedreich's ataxia”; U.S. Pat. No. 9,902,959B2, published Feb. 27, 2018, entitled “Treatment of Frataxin (FXN) related diseases by inhibition of natural antisense transcript to FXN”; U.S. Pat. No. 10,822,369B2, published Nov. 3, 2020, entitled “Compounds and methods for the modulation of proteins”; US Patent Application Publication No. 20220017903A1, published Sep. 27, 2021, entitled “Compositions and methods for treatment of friedreich's ataxia”; International PCT Application Publication No. WO2017186815A1, published Nov. 2, 2017, entitled “Antisense oligonucleotides for enhanced expression of frataxin”; US Patent Application Publication No. 20210087559A1, published Mar. 25, 2021, entitled “Modulation of frataxin expression”; US Patent Application Publication No. 20210285002A1, published Sep. 16, 2021, entitled “Oligonucleotides targeting frataxin and related methods”; the entire contents of each of which are herein incorporated by reference.

Certain oligonucleotides provided in this section may be useful in treating Friedreich's ataxia and neurological disorders associated with FXN by modulating the activity of genes and/or gene products other than FXN genes/gene products, such as DMPK, SMN, and/or DMD genes/gene products.

### Polypeptides

FXN expression and/or activity of the protein encoded thereby in some embodiments can be modulated by the use of polypeptides, such as polypeptides that can interact with FXN nucleic acids and/or its encoded protein (e.g., to modulate its biological activity, its localization within the cell, its aggregation, and/or its interaction with other biomolecules).

In some embodiments, examples of polypeptides (e.g., peptides; proteins, such as enzymes; antibodies; etc.) useful in the treatment of Friedreich's ataxia and neurological disorders associated with FXN are provided in US Patent Application Publication No. 20210292766A1, published Sep. 23, 2021, entitled “Inhibition of Protein Kinases to Treat Friedreich Ataxia”; International PCT Application Publication No. WO2021061698A1, published Apr. 1, 2021, entitled “Methods and compositions for modulating frataxin expression and treating friedrich's ataxia”; the entire contents of each of which are herein incorporated by reference.

Certain polypeptides provided in this section may be useful in Friedreich's ataxia and neurological disorders associated with FXN by modulating the activity of genes and/or gene products other than FXN genes/gene products, such as DMPK, SMN, and/or FXN genes/gene products.

### Small Molecules

FXN expression and/or the activity of the protein it encodes in some embodiments can be modulated by the use of small molecules that can modulate FXN (e.g., to modulate its biological activity, its expression, its localization, its aggregation, and/or its interaction with other biomolecules).

In some embodiments, examples of small molecules useful in the treatment of Friedreich's ataxia and neurological disorders associated with FXN are provided in U.S. Pat. No. 11,124,795B2, published Sep. 21, 2021, entitled “Genetic and pharmacological transcriptional upregulation of the repressed FXN gene as a therapeutic strategy for Friedreich ataxia”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Friedreich's ataxia and neurological disorders associated with FXN by modulating the activity of genes and/or gene products other than FXN genes/gene products, such as DMPK, SMN, and/or DMD genes/gene products.

### Gene Therapies

FXN expression and/or activity in some embodiments can be modulated by the use of gene therapies that can modulate FXN (e.g., by delivery of nucleic acids encoding FXN or other molecules that interact with FXN transcripts or the protein encoded by FXN).

In some embodiments, gene therapies, such as those involving administration of compounds encoding useful therapeutic agents, useful in the treatment of Friedreich's ataxia and neurological disorders associated with FXN are provided in US Patent Application Publication No. 20180327471A1, published Nov. 15, 2018, entitled “Translatable molecules and synthesis thereof”; US Patent Application Publication No. 20210292766A1, published Sep. 23, 2021, entitled “Inhibition of Protein Kinases to Treat Friedreich Ataxia”; U.S. Pat. No. 11,149,256B2, published Oct. 19, 2021, entitled “Adeno-associated virus compositions for targeted gene therapy”; International PCT Application Publication No. WO2021061698A1, published Apr. 1, 2021, entitled “Methods and compositions for modulating frataxin expression and treating friedrich's ataxia”; the entire contents of each of which are herein incorporated by reference.

Certain gene therapies provided in this section may be useful in treating Friedreich's ataxia and neurological disorders associated with FXN by modulating the activity of genes and/or gene products other than FXN genes/gene products, such as DMPK, SMN, and/or DMD genes/gene products.

### Molecular Payloads Targeting SMN

The SMN gene, which encodes survival of motor neuron protein, is associated with neurological disorders. Modulation of SMN and/or survival of motor neuron protein expression and activity (e.g., by suppressing the expression of SMN or mutant forms thereof, and/or activity of the encoded protein) therefore in some embodiments can have a therapeutic effect in subjects with neurological disorders associated with SMN.

### Oligonucleotides

SMN expression and/or activity in some embodiments can be modulated by the use of oligonucleotides targeting SMN sequences.

In some embodiments, an oligonucleotide useful for the treatment of neurological disorders associated with SMN, e.g., targeting (e.g., directly or indirectly modulating the expression or activity of) SMN, comprises a region of complementarity to an SMN1 or SMN2 transcript provided in Table 3, e.g., provided by any one of SEQ ID NOs: 475-496 and 833-836.

In some embodiments, examples of oligonucleotides useful for the treatment of neurological disorders associated with SMN, e.g., targeting (e.g., directly or indirectly modulating the expression or activity of) SMN, are provided in U.S. Pat. No. 10,174,328B2, published Jan. 8, 2019, entitled “Compositions and methods for treating amyotrophic lateral sclerosis”; International PCT Application Publication No. WO2021046254A1, published Mar. 11, 2021, entitled “Liposomal spherical nucleic acid (sna) constructs for splice modulation”; US Patent Application Publication No. 20210228615A1, published Jul. 29, 2021, entitled “Oligonucleotide compositions and methods thereof”; U.S. Pat. No. 9,845,469B2, published Dec. 19, 2017, entitled “Antisense oligonucleotides for treatment of spinal muscular atrophy”; U.S. Pat. No. 10,465,191B2, published Nov. 5, 2019, entitled “Tricyclo-DNA antisense oligonucleotides, compositions, and methods for the treatment of disease”; US Patent Application Publication No. 20120149757A1, published Jun. 14, 2012, entitled “Compositions and methods for modulation of smn2 splicing”; U.S. Pat. No. 9,217,147B2, published Dec. 22, 2015, entitled “Spinal muscular atrophy treatment via targeting SMN2 catalytic core”; US Patent Application Publication No. 20140343127A1, published Nov. 20, 2014, entitled “Compounds for the modulation of smn2 splicing”; U.S. Pat. No. 10,577,605B2, published Mar. 3, 2020, entitled “Induced exon inclusion in spinal muscle atrophy”; U.S. Pat. No. 9,926,559B2, published Mar. 27, 2018, entitled “Compositions and methods for modulation of SMN2 splicing in a subject”; U.S. Pat. No. 9,856,474B2, published Jan. 2, 2018, entitled “Deep intronic target for splicing correction on spinal muscular atrophy gene”; US Patent Application Publication No. 20170051277A1, published Feb. 23, 2017, entitled “Antisense oligomers and methods for treating smn-related pathologies”; U.S. Pat. No. 9,845,469B2, published Dec. 19, 2017, entitled “Antisense oligonucleotides for treatment of spinal muscular atrophy”; US Patent Application Publication No. 20210032624A1, published Feb. 4, 2021, entitled “Compositions and Methods for Modulation of SMN2 Splicing in a Subject”; U.S. Pat. No. 10,851,371B2, published Dec. 1, 2020, entitled “Modulation of SMN expression”; US Patent Application Publication No. 20220073914A1, published Mar. 10, 2022, entitled “Compounds and methods for modulation of smn2”; US Patent Application Publication No. 20210002640A1, published Jan. 7, 2021, entitled “Liposomal spherical nucleic acid (sna) constructs for survival of motor neuron (sma) inhibitors”; US Patent Application Publication No. 20210308281A1, published Oct. 7, 2021, entitled “Combination therapy for spinal muscular atrophy”; International PCT Application Publication No. WO2022015753A1, published Jan. 20, 2022, entitled “Compositions for treatment of spinal muscular atrophy”; the entire contents of each of which are herein incorporated by reference. Additional examples of oligonucleotides useful for the treatment of neurological disorders associated with SMN, e.g., targeting (e.g., directly or indirectly modulating the expression or activity of) SMN, are provided in WO2023102242, published Jun. 8, 2023, entitled “Splice switcher antisense oligonucleotides with modified backbone chemistries”; US20120149757A1, published Jun. 14, 2012, entitled “Compositions and methods for modulation of smn2 splicing”; US20140343127A1, published Nov. 20, 2014, entitled “Compounds for the modulation of smn2 splicing”; U.S. Pat. No. 9,885,040B2, published Feb. 6, 2018, entitled “SMN2 element 1 antisense compositions and methods and uses thereof”; US20190323006A1, published Oct. 24, 2019, entitled “Compounds and methods for modulation of smn2”; U.S. Pat. No. 9,944,926B2, published Apr. 17, 2018, entitled “Induced exon inclusion in spinal muscle atrophy”; US20220064638A1, published Mar. 3, 2022, entitled “Compounds and methods for modulating smn2”; U.S. Pat. No. 8,361,977B2, published Jan. 29, 2013, entitled “Compositions and methods for modulation of SMN2 splicing”; US20070292408A1, published Dec. 20, 2007, entitled “Spinal Muscular Atrophy (SMA) treatment via targeting of SMN2 splice site inhibitory sequences”; US20160002627A1, published Jan. 7, 2016, entitled “Compositions and methods for modulation of smn2 splicing in a subject”; the entire contents of each of which are herein incorporated by reference.

Certain oligonucleotides provided in this section may be useful in treating neurological disorders associated with SMN by modulating the activity of genes and/or gene products other than SMN genes/gene products, such as DMPK, and/or FXN genes/gene products.

### Polypeptides

SMN expression and/or activity of the protein encoded thereby in some embodiments can be modulated by the use of polypeptides, such as polypeptides that can interact with DMD nucleic acids and/or its encoded protein (e.g., to modulate its biological activity, its localization within the cell, its aggregation, and/or its interaction with other biomolecules).

In some embodiments, examples of polypeptides (e.g., peptides; proteins, such as enzymes; antibodies; etc.) useful in the treatment of neurological disorders associated with SMN are provided in U.S. Pat. No. 11,299,537B2, published Apr. 12, 2022, entitled “Methods for treatment of motor neuron diseases”; US Patent Application Publication No. 20190161535A1, published May 30, 2019, entitled “Compositions and methods for treating spinal muscular atrophy”; International PCT Application Publication No. WO2021089736A1, published May 14, 2021, entitled “Combined therapy for muscular diseases”; International PCT Application Publication No. WO2022093724A1, published May 5, 2022, entitled “Use of anti-pro/latent myostatin antibody for treating spinal muscular atrophy”; the entire contents of each of which are herein incorporated by reference. Certain polypeptides provided in this section may be useful in treating neurological disorders associated with SMN by modulating the activity of genes and/or gene products other than SMN genes/gene products, such as DMPK, and/or FXN genes/gene products.

### Small Molecules

SMN expression and/or the activity of the protein it encodes in some embodiments can be modulated by the use of small molecules that can modulate SMN (e.g., to modulate its biological activity, its expression, its localization, its aggregation, and/or its interaction with other biomolecules).

In some embodiments, examples of small molecules useful in the treatment of neurological disorders associated with SMN are provided in U.S. Pat. No. 8,729,263B2, published May 20, 2014, entitled “1,4-disubstituted pyridazine analogs there of and methods for treating SMN-deficiency-related conditions”; U.S. Pat. No. 10,874,672B2, published Dec. 29, 2020, entitled “Methods for treating Huntington's disease”; International PCT Application Publication No. WO2017218905A1, published Dec. 21, 2017, entitled “A method for treating spinal muscular atrophy”; International PCT Application Publication No. WO2020234496A1, published Nov. 26, 2020, entitled “Moxifloxacin for use in the treatment of spinal muscular atrophy”; US Patent Application Publication No. 20220168307A1, published Jun. 2, 2022, entitled “Treatment of sma”; International PCT Application Publication No. WO2022015753A1, published Jan. 20, 2022, entitled “Compositions for treatment of spinal muscular atrophy”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating neurological disorders associated with SMN by modulating the activity of genes and/or gene products other than SMN genes/gene products, such as DMPK, and/or FXN genes/gene products.

### Gene Therapies

SMN expression and/or activity in some embodiments can be modulated by the use of gene therapies that can modulate SMN (e.g., by delivery of nucleic acids encoding SMN or other molecules that interact with SMN transcripts or the protein encoded by SMN).

In some embodiments, gene therapies, such as those involving administration of compounds encoding useful therapeutic agents, useful in the treatment of neurological disorders associated with SMN are provided in U.S. Pat. No. 10,357,543B2, published Jul. 23, 2019, entitled “Methods and compositions for treating disorders and diseases using Survival Motor Neuron (SMN) protein”; US Patent Application Publication No. 20210308281A1, published Oct. 7, 2021, entitled “Combination therapy for spinal muscular atrophy”; International PCT Application Publication No. WO2021089736A1, published May 14, 2021, entitled “Combined therapy for muscular diseases”; the entire contents of each of which are herein incorporated by reference.

Certain gene therapies provided in this section may be useful in treating neurological disorders associated with SMN by modulating the activity of genes and/or gene products other than SMN genes/gene products, such as DMPK, and/or FXN genes/gene products.

### Molecular Payloads for the Treatment of Alzheimer's Disease

Various molecular payloads may be useful in the treatment of Alzheimer's disease, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of Alzheimer's disease may include, in some embodiments, molecular payloads which modulate (e.g., increase or decrease) expression or activity of TREM2, APOE, MAPT, and/or APP.

Examples of oligonucleotides useful for the treatment of Alzheimer's disease, e.g., oligonucleotides targeting (e.g., directly or indirectly modulating the expression or activity of) genes associated with Alzheimer's disease (e.g., TREM2, APOE, MAPT, APP, etc.), include those listed in Table 12 below. Each oligonucleotide provided in Table 12 may have any modification pattern disclosed herein.

TABLE 12
Oligonucleotides for the treatment of Alzheimer′s disease

| | | Oligo- | |
|---|---|---|---|
| SEQ | | nucleotide | |
| ID | Target | Sequence | |
| NO: | gene | (5′-3′) | Oligonucleotide Structure (5′-3′) |
| 142 | TREM2 | ATGGAGTGTA | Aes Tes Ges Ges Aes Gds Tds Gds Tds Ads Tds mCds |
| | | TCCCCGACGC | mCds mCds mCds Ges Aes mCes Ges mCe, wherein mC = 5- |
| | | | methyl cytosine, e = 2′-MOE modified sugar, d = |
| | | | 2′deoxyribose sugar, s = phosphorothioate internucleoside |
| | | | linkage |
| | TREM2 | | siRNA |
| | | | sense strand: CCCUCUAGAUGACCAAGAUtt (SEQ ID NO: 158), |
| | | | antisense strand: AUCUUGGUCAUCUAGAGGGtc (SEQ ID NO: 159) |
| | TREM2 | | siRNA |
| | | | sense strand: GCGUUCUCCUGAGCAAGUUtt (SEQ ID NO: 160), |
| | | | antisense strand: AACUUGCUCAGGAGAACGCag (SEQ ID NO: 161) |
| | TREM2 | | siRNA |
| | | | sense strand: GCACCUCCAGGAAUCAAGAtt (SEQ ID NO: 162), |
| | | | antisense strand: UCUUGAUUCCUGGAGGUGCtg (SEQ ID NO: 163) |
| 143 | TREM2 | CCTGTTCCAG | CCTGTTCCAGGCCTCATGTTTTGGG (SEQ ID NO: 143), wherein |
| | | GCCTCATGTT | the oligonucleotide is a PMO |
| | | TTGGG | |
| 144 | TREM2 | GGGCGTCTGT | GGGCGTCTGTGTGCAGAACCACCCA (SEQ ID NO: 144), wherein |
| | | GTGCAGAACC | the oligonucleotide is a PMO |
| | | ACCCA | |
| 145 | TREM2 | AGTGGCTGCC | AGTGGCTGCCTCTCTGGCCTGCC (SEQ ID NO: 145), wherein |
| | | TCTCTGGCCT | the oligonucleotide is a PMO |
| | | GCC | |
| 146 | TREM2 | TCTGGCCTGC | TCTGGCCTGCCCCTGTTCCAGGC (SEQ ID NO: 146), wherein |
| | | CCCTGTTCCA | the oligonucleotide is a PMO |
| | | GGC | |
| 147 | TREM2 | GGCAGGCCAG | GGCAGGCCAGAGAGGCAGCCACT (SEQ ID NO: 147), wherein |
| | | AGAGGCAGCC | the oligonucleotide is a PMO |
| | | ACT | |
| 148 | TREM2 | GCCTGGAACA | GCCTGGAACAGGGGCAGGCCAGA (SEQ ID NO: 148), wherein the |
| | | GGGGCAGGCC | oligonucleotide is a PMO |
| | | AGA | |
| | APOE | | siRNA with sense strand: GAC CAU GAA GGA GUU GAA Gtt |
| | | | (SEQ ID NO: 170) |
| | APOE | | siRNA with target sequence: AAG GTG GAG CAA GCG GTG |
| | | | GAG (SEQ ID NO: 171) |
| | APOE | | siRNA with target sequence: GGA GTT GAA GGC CTA CAA A |
| | | | (SEQ ID NO: 172) |
| | APOE | | siRNA with target sequence: AAG CAG TTT GCC TGC AGA |
| | | | GAT (SEQ ID NO: 173) |
| | MAPT | | IONIS-MAPT_{Rx} (BIIB080) |
| 149 | MAPT | CCGTTTTCTT | mCes mCeo Ges Tes Tes Tds Tds mCds Tds Tds Ads |
| | | ACCACCCT | mCds mCds Aes mCeo mCes mCes Te; wherein mC = a 5- |
| | | | methylcytosine, e = a 2′-MOE nucleoside, d = a 2′- |
| | | | deoxynucleoside, |
| | | | s = a phosphorothioate internucleoside linkage, and o = a |
| | | | phosphodiester internucleoside linkage. |
| 150 | APP | GCATTCTCTT | Ges mCeo Aeo Teo Tes mCds Tds mCds Tds Tds Ads |
| | | ATATTCCTTA | Tds Ads Tds Tds mCeo mCeo Tes Tes Ae; wherein mC = a |
| | | | 5-methylcytosine, e = a 2′-MOE nucleoside, d = a 2′- |
| | | | deoxynucleoside, |
| | | | s = a phosphorothioate internucleoside linkage, and o = a |
| | | | phosphodiester internucleoside linkage. |
| 151 | APP | GTTTACCTTT | Ges Teo Teo Teo Aes mCds mCds Tds Tds Tds Ads Ads |
| | | AACATTCCTC | mCds Ads Tds Teo mCeo mCes Tes mCe; wherein mC = a |
| | | | 5-methylcytosine, e = a 2′-MOE nucleoside, d = a 2′- |
| | | | deoxynucleoside, |
| | | | s = a phosphorothioate internucleoside linkage, and o = a |
| | | | phosphodiester internucleoside linkage. |
| 152 | APP | GCCATATTGT | Ges mCeo mCeo Aeo Tes Ads Tds Tds Gds Tds mCds |
| | | CATTTTACAC | Ads Tds Tds Tds Teo Aeo mCes Aes mCe; wherein mC = a |
| | | | 5-methylcytosine, e = a 2′-MOE nucleoside, d = a 2′- |
| | | | deoxynucleoside, |
| | | | s = a phosphorothioate internucleoside linkage, and o = a |
| | | | phosphodiester internucleoside linkage. |
| 153 | APP | GTATCCTCTT | Ges Teo Aeo Teo mCes mCds Tds mCds Tds Tds Ads |
| | | AATTCCTATA | Ads Tds Tds mCds mCeo Teo Aes Tes Ae; wherein mC = a |
| | | | 5-methylcytosine, e = a 2′-MOE nucleoside, d = a 2′- |
| | | | deoxynucleoside, |
| | | | s = a phosphorothioate internucleoside linkage, and o = a |
| | | | phosphodiester internucleoside linkage. |
| 154 | APP | CTCCAATTTT | mCes Teo mCeo mCeo Aes Ads Tds Tds Tds Tds Ads |
| | | AACTTGCACC | Ads mCds Tds Tds Geo mCeo Aes mCes mCe; wherein mC = |
| | | | a 5-methylcytosine, e = a 2′-MOE nucleoside, d = a 2′- |
| | | | deoxynucleoside, |
| | | | s = a phosphorothioate internucleoside linkage, and o = a |
| | | | phosphodiester internucleoside linkage. |
| 155 | APP | GTTCACAGTT | Ges Teo Teo mCeo Aes mCds Ads Gds Tds Tds Tds Ads |
| | | TACCCCAAGC | mCds mCds mCds mCeo Aeo Aes Ges mCe; wherein mC = a |
| | | | 5-methylcytosine, e = a 2′-MOE nucleoside, d = a 2′- |
| | | | deoxynucleoside, |
| | | | s = a phosphorothioate internucleoside linkage, and o = a |
| | | | phosphodiester internucleoside linkage. |
| 156 | APP | CTGCAAAGAA | mCes Tes Ges mCes Aes Aes Aes Ges Aes Aes mCes |
| | | CACCTTGA | Aes mCes mCes Tes Tes Ges Ae; wherein mC = a 5- |
| | | | methylcytosine, e = a 2′-MOE nucleoside, s = a |
| | | | phosphorothioate internucleoside linkage. |
| 157 | APP | ATCTTCTGCA | Aes Tes mCes Tes Tes mCes Tes Ges mCes Aes Aes |
| | | AAGAACAC | Aes Ges Aes Aes mCes Aes mCe; wherein mC = a 5- |
| | | | methylcytosine, e = a 2′-MOE nucleoside, s = a |
| | | | phosphorothioate internucleoside linkage. |

In each sequence listed in Table 12, each T may be optionally and independently replaced with a U, and each U may be optionally and independently replaced with a T.

Examples of small molecules useful for the treatment of Alzheimer's disease include:

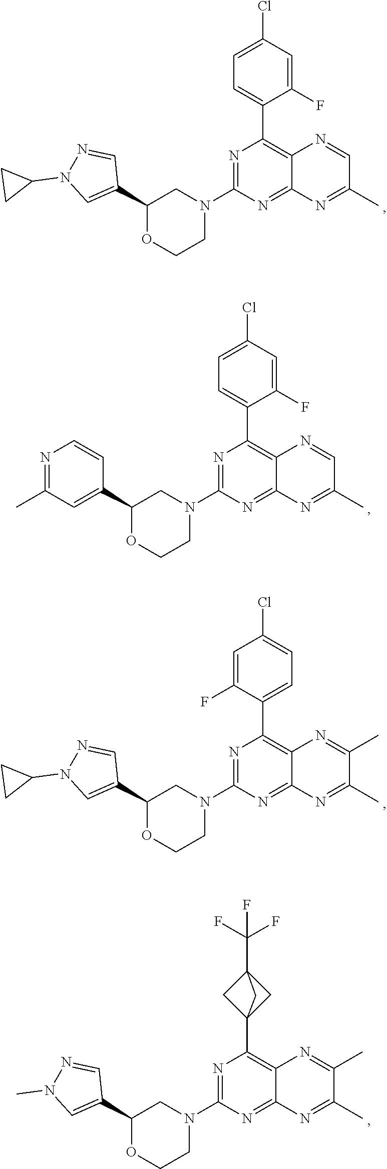

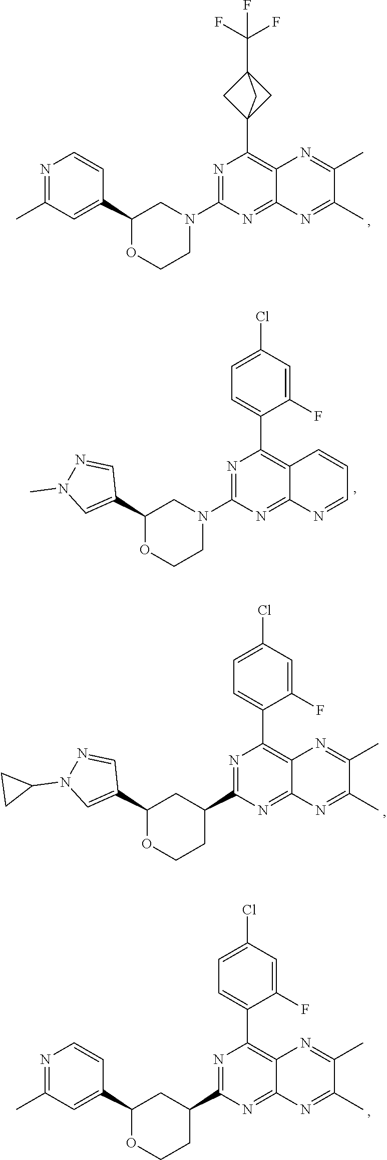

3a-hydroxy-5a-pregnan-20-one,

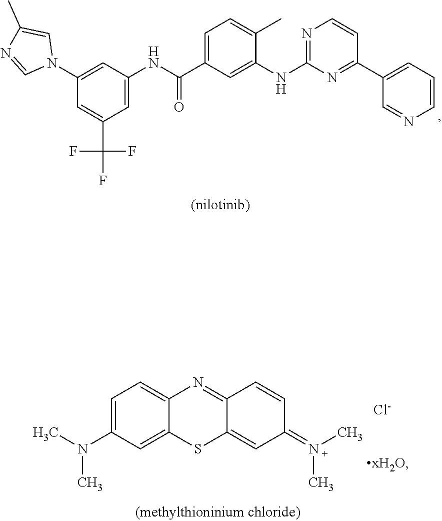

and pharmaceutically acceptable salts, co-crystals, tautomers, stereoisomers, solvates, hydrates, polymorphs, isotopically enriched compounds, and prodrugs thereof.

Additional examples of small molecules useful for the treatment of Alzheimer's disease include:

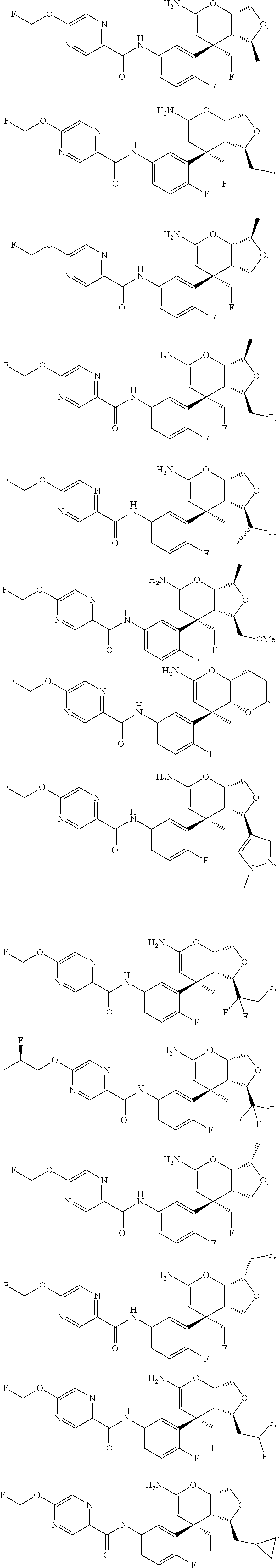

and pharmaceutically acceptable salts, co-crystals, tautomers, stereoisomers, solvates, hydrates, polymorphs, isotopically enriched compounds, and prodrugs thereof.

Examples of polypeptides useful for the treatment of Alzheimer's disease include antibodies, proteins, peptides, and enzymes. In some embodiments, a polypeptide useful for the treatment of Alzheimer's disease comprises AP 12-28P (peptide with amino acid sequence VHHQKLPFFAEDVGSNK, SEQ ID NO: 174), KEESIYCRLMGLGCG (SEQ ID NO: 175), NELSPYCRLMGLGCD (SEQ ID NO: 176), NEESMYCRLLGIGCG (SEQ ID NO: 177), PEESLYCRLLALGCG (SEQ ID NO: 178), SMYCRLLGIGCG (SEQ ID NO: 179), ESMYCRLLGIGCG (SEQ ID NO: 180), Bapineuzumab, Solanezumab, gantenerumab, solanezumab, gantenerumab, crenezumab, ponezumab, lecanemab, or Aducanumab. In some embodiments, a polypeptide useful for the treatment of Alzheimer's disease comprises trontinemab.

### Molecular Payloads Targeting TREM2

The TREM2 gene, and mutations therein, are implicated in Alzheimer's disease, which predominantly affects neurons in the brain. Modulation of TREM2 expression and activity (e.g., by suppressing the expression of TREM2 or a mutant form thereof and/or activity of a wild-type or mutant form of a protein encoded by TREM2) therefore in some embodiments can have a therapeutic effect in subjects with Alzheimer's disease.

### Oligonucleotides

TREM2 expression and/or activity in some embodiments can be modulated by the use of oligonucleotides targeting TREM2 sequences.

In some embodiments, an oligonucleotide useful for the treatment of Alzheimer's disease, e.g., targeting (e.g., directly or indirectly modulating the expression or activity of) TREM2, comprises a region of complementarity to a TREM2 transcript provided in Table 3, e.g., provided by any one of SEQ ID NOs: 499-500.

In some embodiments, examples of oligonucleotides useful for the treatment of Alzheimer's disease, e.g., targeting (e.g., directly or indirectly modulating the expression or activity of) TREM2, are provided in Schoch K M, et al. “Acute Trem2 reduction triggers increased microglial phagocytosis, slowing amyloid deposition in mice.” Proc Natl Acad Sci USA. (2021) 118(27):e2100356118; Hu Y, et al. “TREM2, Driving the Microglial Polarization, Has a TLR4 Sensitivity Profile After Subarachnoid Hemorrhage.” Front Cell Dev Biol. (2021) 9:693342; Liu W, et al. “Trem2 promotes anti-inflammatory responses in microglia and is suppressed under pro-inflammatory conditions.” Hum Mol Genet. (2020) 29(19):3224-3248; and International Patent Application Publication No. WO2022035984A1, published Feb. 17, 2022, entitled “Antisense oligonucleotides for treatment of conditions and diseases related to trem2”; the entire contents of each of which are herein incorporated by reference.

Certain oligonucleotides provided in this section may be useful in treating Alzheimer's disease by modulating the activity of genes and/or gene products other than TREM2 genes/gene products, such as APOE, MAPT, and/or APP genes/gene products.

### Polypeptides

TREM2 expression and/or activity in some embodiments can be modulated by the use of TREM2 polypeptides or polypeptides that can interact with TREM2 (e.g., to modulate its activity).

In some embodiments, examples of polypeptides (e.g., peptides; proteins, such as enzymes; antibodies; etc.) useful in the treatment of Alzheimer's disease are provided in U.S. Patent Application Publication No. US2022127356A1, published Apr. 4, 2022, entitled “Trem2 antibodies and uses thereof”; U.S. Patent Application Publication No. US20210054069A1, published Nov. 30, 2021, entitled “TREM2 Antigen Binding Proteins And Uses Thereof”; U.S. Patent Application Publication No. US20200317776A1, published Oct. 8, 2020, entitled “Anti-trem2 antibodies and methods of use thereof”; U.S. Patent Application Publication No. US2021214438A1, published Sep. 21, 2021, entitled “Anti-trem2 antibodies and methods of use thereof”; International Patent Application Publication No. WO2022120390A1, published Jun. 9, 2022, entitled “Treatment of diseases related to atp-binding cassette transporter 1 dysfunction using trem2 agonists”; U.S. Patent Application Publication No. US20220089726A1, published Mar. 24, 2022, entitled “Treatment of diseases related to colony-stimulating factor 1 receptor dysfunction using trem2 agonists”; International Patent Application Publication No. WO2021113655A1, published Jun. 10, 2021, entitled “Methods of use of anti-trem2 antibodies”; U.S. Patent Application Publication No. US2021054069A1, published Nov. 30, 2021, entitled “TREM2 Antigen Binding Proteins And Uses Thereof”; U.S. Patent Application Publication No. US20220119522A1, published Apr. 21, 2022, entitled “Anti-trem2 antibodies and methods of use thereof”; U.S. Pat. No. 10,040,836B2, published Aug. 7, 2018, entitled “Peptides for the treatment of neurodegenerative diseases”; U.S. Patent Application Publication No. US20220073609A1, published Mar. 10, 2022, entitled “Anti-trem2 antibodies and methods of use thereof”; U.S. Patent Application Publication No. US2020140545A1, published May 7, 2020, entitled “Trem2 stabilizing antibodies”; U.S. Patent Application Publication No. US20200277373A1, published Sep. 3, 2020, entitled “Anti-trem2 antibodies and methods of use thereof”; U.S. Patent Application Publication No. US2019309064A1, published Dec. 17, 2019, entitled “Anti-trem2 antibodies and related methods”; U.S. Patent Application Publication No. US20190330335A1, published Oct. 21, 2019, entitled “Anti-trem2 antibodies and methods of use thereof”; U.S. Patent Application Publication No. US2017240631A1, published Aug. 24, 2017, entitled “Anti-trem2 antibodies and methods of use thereof”; U.S. Patent Application Publication No. US20190010230A1, published Jan. 10, 2019, entitled “Anti-trem2 antibodies and methods of use thereof”; and U.S. Patent Application Publication No. US20190185565A1, published Jun. 20, 2019, entitled “Trem2 cleavage modulators and uses thereof”; the entire contents of each of which are herein incorporated by reference.

Certain polypeptides provided in this section may be useful in treating Alzheimer's disease by modulating the activity of genes and gene products other than TREM2 genes/gene products, such as APOE, MAPT, and/or APP genes/gene products.

### Small Molecules

TREM2 expression and/or activity in some embodiments can be modulated by the use of small molecules that can modulate TREM2 (e.g., to modulate its activity, or its expression).

In some embodiments, examples of small molecules useful in the treatment of Alzheimer's disease are provided in International Patent Application Publication No. WO2021226629A1, published Nov. 11, 2021, entitled “Heterocyclic compounds as triggering receptor expressed on myeloid cells 2 agonists and methods of use”; the entire contents of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Alzheimer's disease by modulating the activity of genes and gene products other than TREM2 genes/gene products, such as APOE, MAPT, and/or APP genes/gene products.

### Gene Therapies

TREM2 expression and/or activity in some embodiments can be modulated by the use of gene therapies that can modulate TREM2 (e.g., by delivery of nucleic acids encoding TREM2 or other molecules that interact with TREM2).

In some embodiments, gene therapies, such as those involving administration of compounds encoding useful therapeutic agents, useful in the treatment of Alzheimer's disease are provided in International Patent Application Publication No. WO2022006105A2, published Jan. 6, 2022, entitled “Compositions and methods for treating neurocognitive disorders”; International Patent Application Publication No. WO2021067611A2, published Apr. 8, 2021, entitled “Compositions and methods for treating Alzheimer's disease”; U.S. Patent Application Publication No. US20210162072A1, published Jun. 3, 2021, entitled “Modified adeno-associated virus vectors and delivery thereof into the central nervous system”; U.S. Patent Application Publication No. US2020207830A1, published Jul. 2, 2020, entitled “Trem2 mutants resistant to sheddase cleavage”; U.S. Patent Application Publication No. US2021195879A1, published Jul. 1, 2021, entitled “Genetically modified mouse models of Alzheimer's disease”; and U.S. Patent Application Publication No. US20190048057A1, published Feb. 14, 2019, entitled “Compositions comprising trem2 and methods of use thereof”; the entire contents of each of which are herein incorporated by reference. Certain gene therapies provided in this section may be useful in treating Alzheimer's disease by modulating the activity of genes and gene products other than TREM2 genes/gene products, such as APOE, MAPT, and/or APP genes/gene products.

### Molecular Payloads Targeting APOE

The APOE gene, and mutations therein, are implicated in Alzheimer's disease, which predominantly affects neurons in the brain. Modulation of APOE expression and activity (e.g., by suppressing the expression of APOE or a mutant form thereof and/or activity of a wild-type or mutant form of a protein encoded by APOE) therefore in some embodiments can have a therapeutic effect in subjects with Alzheimer's disease.

### Oligonucleotides

APOE expression and/or activity in some embodiments can be modulated by the use of oligonucleotides targeting APOE sequences.

In some embodiments, an oligonucleotide useful for the treatment of Alzheimer's disease, e.g., targeting (e.g., directly or indirectly modulating the expression or activity of) APOE, comprises a region of complementarity to an APOE transcript provided in Table 3, e.g., provided by any one of SEQ ID NOs: 501-505.

In some embodiments, examples of oligonucleotides useful for the treatment of Alzheimer's disease, e.g., targeting (e.g., directly or indirectly modulating the expression or activity of) APOE, are provided in Barger S W, et al. “Relationships between expression of apolipoprotein E and beta-amyloid precursor protein are altered in proximity to Alzheimer beta-amyloid plaques: potential explanations from cell culture studies.” J Neuropathol Exp Neurol. (2008) 67(8):773-83; Casey C S, et al. “Apolipoprotein E Inhibits Cerebrovascular Pericyte Mobility through a RhoA Protein-mediated Pathway.” J Biol Chem. (2015) 290(22):14208-17; International Patent Application Publication No. WO2022066956A1, published Mar. 31, 2022, entitled “Compounds and methods for reducing apoe expression”; International Patent Application Publication No. WO2021178374A2, published Sep. 10, 2021, entitled “Compounds and methods for reducing apoe expression”; U.S. Patent Application Publication No. US20210155925A1, published May 27, 2021, entitled “Compositions and methods for disrupting the molecular mechanisms associated with mitochondrial dysfunction and neurodegenerative disease”; and U.S. Patent Application Publication No. US20170334977A1, published Nov. 23, 2017, entitled “Targeting Apolipoprotein E (APOE) in Neurologic Disease”; the entire contents of each of which are herein incorporated by reference.

Certain oligonucleotides provided in this section may be useful in treating Alzheimer's disease by modulating the activity of genes and/or gene products other than APOE genes/gene products, such as TREM2, MAPT, and/or APP genes/gene products.

### Polypeptides

APOE expression and/or activity in some embodiments can be modulated by the use of APOE polypeptides or polypeptides that can interact with APOE (e.g., to modulate its activity).

In some embodiments, examples of polypeptides (e.g., peptides; proteins, such as enzymes; antibodies; etc.) useful in the treatment of Alzheimer's disease are provided in Sadowski M J, at al. “Blocking the apolipoprotein E/amyloid-beta interaction as a potential therapeutic approach for Alzheimer's disease.” Proc Natl Acad Sci USA. (2006) 103(49):18787-92; U.S. Patent Application Publication No. US20040214774A1, published Oct. 28, 2004, entitled “Prevention and treatment of Alzheimer amyloid deposition”; International Patent Application Publication No. WO2020243346A1, published Dec. 3, 2020, entitled “Apoe antibodies, fusion proteins and uses thereof”; U.S. Patent Application Publication No. US20190270794A1, published Sep. 21, 2021, entitled “Anti-apoe antibodies”; U.S. Pat. No. 10,040,836B2, published Aug. 7, 2018, entitled “Peptides for the treatment of neurodegenerative diseases”; U.S. Patent Application Publication No. US2014037638A1, published Feb. 6, 2014, entitled “Compositions and methods for treating amyloid plaque associated symptoms”; and U.S. Patent Application Publication No. US20100266505A1, published Oct. 21, 2010, entitled “Immunotherapy regimes dependent on apoe status”; the entire contents of each of which are herein incorporated by reference.

Certain polypeptides provided in this section may be useful in treating Alzheimer's disease by modulating the activity of genes and gene products other than APOE genes/gene products, such as TREM2, MAPT, and/or APP genes/gene products.

### Small Molecules

APOE expression and/or activity in some embodiments can be modulated by the use of small molecules that can modulate APOE (e.g., to modulate its activity, or its expression).

In some embodiments, examples of small molecules useful in the treatment of Alzheimer's disease are provided in U.S. Patent Application Publication No. US20210353566A1, published Nov. 18, 2021, entitled “The use of choline supplementation as therapy for apoe4-related disorders”; U.S. Patent Application Publication No. US20210085692A1, published Mar. 25, 2021, entitled “Agents, compositions and methods for treating and preventing Alzheimer's disease”; and U.S. Patent Application Publication No. US20180036274A1, published Oct. 30, 2018, entitled “Use of medium chain triglycerides for the treatment and prevention of Alzheimer's disease and other diseases resulting from reduced neuronal metabolism ii”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Alzheimer's disease by modulating the activity of genes and gene products other than APOE genes/gene products, such as TREM2, MAPT, and/or APP genes/gene products.

### Gene Therapies

APOE expression and/or activity in some embodiments can be modulated by the use of gene therapies that can modulate APOE (e.g., by delivery of nucleic acids encoding APOE or other molecules that interact with APOE).

In some embodiments, gene therapies, such as those involving administration of compounds encoding useful therapeutic agents, useful in the treatment of Alzheimer's disease are provided in International Patent Application Publication No. WO2021067611A2, published Apr. 8, 2021, entitled “Compositions and methods for treating Alzheimer's disease”; and U.S. Patent Application Publication No. US2021195879A1, published Jul. 1, 2021, entitled “Genetically modified mouse models of Alzheimer's disease”; the entire contents of each of which are herein incorporated by reference.

Certain gene therapies provided in this section may be useful in treating Alzheimer's disease by modulating the activity of genes and gene products other than APOE genes/gene products, such as TREM2, MAPT, and/or APP genes/gene products.

### Molecular Payloads Targeting MAPT

The MAPT gene, and mutations therein, are implicated in Alzheimer's disease, which predominantly affects neurons in the brain. Modulation of MAPT expression and activity (e.g., by suppressing the expression of MAPT or a mutant form thereof and/or activity of a wild-type or mutant form of a protein encoded by MAPT) therefore in some embodiments can have a therapeutic effect in subjects with Alzheimer's disease.

### Oligonucleotides

MAPT expression and/or activity in some embodiments can be modulated by the use of oligonucleotides targeting MAPT sequences.

In some embodiments, an oligonucleotide useful for the treatment of Alzheimer's disease, e.g., targeting (e.g., directly or indirectly modulating the expression or activity of) MAPT, comprises a region of complementarity to a MAPT transcript provided in Table 3, e.g., provided by any one of SEQ ID NOs: 506-534.

In some embodiments, examples of oligonucleotides useful for the treatment of Alzheimer's disease, e.g., targeting (e.g., directly or indirectly modulating the expression or activity of) MAPT, are provided in International Patent Application Publication No. WO2022077024A1, published Apr. 14, 2022, entitled “Selective delivery of oligonucleotides to glial cells”; International Patent Application Publication No. WO2022009987A1, published Jan. 13, 2022, entitled “Method for treating Alzheimer's disease by targeting mapt gene”; International Patent Application Publication No. WO2021202511A2, published Oct. 7, 2021, entitled “Microtubule associated protein tau (MAPT) iRNA agent compositions and methods of use thereof”; U.S. Pat. No. 11,053,498B2, published Jul. 6, 2021, entitled “Compounds and methods for reducing Tau expression”; U.S. Pat. No. 10,273,474B2, published Apr. 30, 2019, entitled “Methods for modulating Tau expression for reducing seizure and modifying a neurodegenerative syndrome”; U.S. Patent Application Publication No. US20180161356A1, published Jun. 14, 2018, entitled “Tau antisense oligomers and uses thereof”; U.S. Patent Application Publication No. US2021363523A1, published Nov. 25, 2021, entitled “Oligonucleotides for mapt modulation”; International Patent Application Publication No. WO2021178237A2, published Sep. 10, 2021, entitled “Oligonucleotide compositions and methods thereof”; U.S. Patent Application Publication No. US20200010831A1, published Mar. 22, 2022, entitled “Oligonucleotides for modulating tau expression”; U.S. Patent Application Publication No. US2018066254A1, published Dec. 24, 2019, entitled “Rna interference mediated therapy for neurodegenerative diseases”; U.S. Patent Application Publication No. US2016237427A1, published Oct. 13, 2020, entitled “Tau Antisense Oligomers and Uses Thereof”; U.S. Pat. No. 11,332,733B2, published May 17, 2022, entitled “Modified compounds and uses thereof”; and U.S. Pat. No. 9,644,207B2, published May 9, 2017, entitled “Compositions and methods for modulating tau expression”; the entire contents of each of which are herein incorporated by reference.

Certain oligonucleotides provided in this section may be useful in treating Alzheimer's disease by modulating the activity of genes and/or gene products other than MAPT genes/gene products, such as TREM2, APOE, and/or APP genes/gene products.

### Polypeptides

MAPT expression and/or activity in some embodiments can be modulated by the use of MAPT polypeptides or polypeptides that can interact with MAPT (e.g., to modulate its activity).

In some embodiments, examples of polypeptides (e.g., peptides; proteins, such as enzymes; antibodies; etc.) useful in the treatment of Alzheimer's disease are provided in International Patent Application Publication No. WO2021151012A1, published Jul. 29, 2021, entitled “Zinc finger protein transcription factors for repressing tau expression”; U.S. Patent Application Publication No. US20220146535A1, published May 12, 2022, entitled “Compounds and methods targeting human tau”; U.S. Patent Application Publication No. US20220127345A1, published Apr. 28, 2022, entitled “Methods of Reducing Tau in Human Subjects”; U.S. Patent Application Publication No. US20210163582A1, published Oct. 19, 2021, entitled “Antibodies that bind to pathological tau species and uses thereof”; International Patent Application Publication No. WO2021262791A1, published Dec. 30, 2021, entitled “High affinity antibodies targeting tau phosphorylated at serine 413”; U.S. Patent Application Publication No. US20200216522A1, published Jul. 9, 2020, entitled “Anti-tau antibodies and methods of use thereof”; U.S. Patent Application Publication No. US20200131255A1, published Apr. 30, 2020, entitled “Methods of treating neurodegenerative diseases”; U.S. Patent Application Publication No. US20190135905A1, published May 9, 2019, entitled “Compositions and methods for treating tauopathies”; U.S. Patent Application Publication No. US2018194832A1, published Jul. 12, 2018, entitled “Tau-binding antibodies”; U.S. Patent Application Publication No. US20190135905A1, published May 9, 2019, entitled “Compositions and methods for treating tauopathies”; U.S. Pat. No. 10,040,836B2, published Aug. 7, 2018, entitled “Peptides for the treatment of neurodegenerative diseases”; U.S. Patent Application Publication No. US20180201666A1, published Jul. 19, 2018, entitled “Tau-binding antibodies”; U.S. Patent Application Publication No. US20190135905A1, published May 9, 2019, entitled “Compositions and methods for treating tauopathies”; U.S. Patent Application Publication No. US20180142007A1, published May 24, 2018, entitled “Humanized tau antibodies in Alzheimer's disease”; U.S. Patent Application Publication No. US20170296680A1, published Oct. 19, 2017, entitled “Anti-tau antibody and uses thereof”; U.S. Patent Application Publication No. US20170152307A1, published Jun. 1, 2017, entitled “Antibodies and antigen-binding fragments that specifically bind to microtubule-associated protein tau”; U.S. Patent Application Publication No. US20170015738A1, published Jan. 19, 2017, entitled “Antibodies Specific for Hyperphosphorylated Tau and Methods of Use Thereof”; U.S. Patent Application Publication No. US20160376351A1, published Dec. 29, 2016, entitled “Anti-tau antibodies and methods of use”; U.S. Patent Application Publication No. US20150183855A1, published Jul. 2, 2015, entitled “Antibodies to tau”; U.S. Patent Application Publication No. US20150344553A1, published Dec. 3, 2015, entitled “Human anti-tau antibodies”; and U.S. Patent Application Publication No. US20120087861A1, published Apr. 12, 2012, entitled “Human Anti-Tau Antibodies”; the entire contents of each of which are herein incorporated by reference.

Certain polypeptides provided in this section may be useful in treating Alzheimer's disease by modulating the activity of genes and gene products other than MAPT genes/gene products, such as TREM2, APOE, and/or APP genes/gene products.

### Small Molecules

MAPT expression and/or activity in some embodiments can be modulated by the use of small molecules that can modulate MAPT (e.g., to modulate its activity, or its expression).

In some embodiments, examples of small molecules useful in the treatment of Alzheimer's disease are provided in Turner R S, et al. “Nilotinib Effects on Safety, Tolerability, and Biomarkers in Alzheimer's Disease.” Ann Neurol (2020) 88(1):183-194; Melis V, et al. “Effects of oxidized and reduced forms of methylthioninium in two transgenic mouse tauopathy models.” Behav Pharmacol. (2015) 26(4):353-68; International Patent Application Publication No. WO2022078971A1, published Apr. 21, 2022, entitled “Novel compounds”; and International Patent Application Publication No. WO2019134978A1, published Jul. 11, 2019, entitled “1, 3, 4, 5-tetrahydro-2 h-pyrido[4,3-b]indole derivatives for the treatment, alleviation or prevention of disorders associated with tau aggregates like Alzheimer's disease”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Alzheimer's disease by modulating the activity of genes and gene products other than MAPT genes/gene products, such as TREM2, APOE, and/or APP genes/gene products.

### Gene Therapies

MAPT expression and/or activity in some embodiments can be modulated by the use of gene therapies that can modulate MAPT (e.g., by delivery of nucleic acids encoding MAPT or other molecules that interact with MAPT).

In some embodiments, gene therapies, such as those involving administration of a compounds encoding useful therapeutic agents, useful in the treatment of Alzheimer's disease are provided in International Patent Application Publication No. WO2021151012A1, published Jul. 29, 2021, entitled “Zinc finger protein transcription factors for repressing tau expression”; the entire contents of which are herein incorporated by reference.

Certain gene therapies provided in this section may be useful in treating Alzheimer's disease by modulating the activity of genes and gene products other than MAPT genes/gene products, such as TREM2, APOE, and/or APP genes/gene products.

### Molecular Payloads Targeting APP

The APP gene, and mutations therein, are implicated in Alzheimer's disease, which predominantly affects neurons in the brain. Modulation of APP expression and activity (e.g., by suppressing the expression of APP or a mutant form thereof and/or activity of a wild-type or mutant form of a protein encoded by APP) therefore in some embodiments can have a therapeutic effect in subjects with Alzheimer's disease.

### Oligonucleotides

APP expression and/or activity in some embodiments can be modulated by the use of oligonucleotides targeting APP sequences.

In some embodiments, an oligonucleotide useful for the treatment of Alzheimer's disease, e.g., targeting (e.g., directly or indirectly modulating the expression or activity of) APP, comprises a region of complementarity to an APP transcript provided in Table 3, e.g., provided by any one of SEQ ID NOs: 535-545.

In some embodiments, examples of oligonucleotides useful for the treatment of Alzheimer's disease, e.g., targeting (e.g., directly or indirectly modulating the expression or activity of) APP, are provided in International Patent Application Publication No. WO2022026589A1, published Feb. 3, 2022, entitled “Compounds and methods for reducing app expression”; International Patent Application Publication No. WO2020160163A1, published Aug. 6, 2020, entitled “Compounds and methods for reducing app expression”; U.S. Patent Application Publication No. US20210040480A1, published Feb. 11, 2021, entitled “Compounds and methods for the modulation of amyloid-beta precursor protein”; U.S. Patent Application Publication No. US20210017513A1, published Jan. 21, 2021, entitled “Modified compounds and uses thereof”; and U.S. Patent Application Publication No. US20130046007A1, published Feb. 21, 2013, entitled “Selective reduction of allelic variants”; U.S. Pat. No. 10,900,041B2, published Jan. 26, 2021, entitled “Antisense oligonucleotides for use in treating Alzheimer's disease”; the entire contents of each of which are herein incorporated by reference.

Certain oligonucleotides provided in this section may be useful in treating Alzheimer's disease by modulating the activity of genes and/or gene products other than APP genes/gene products, such as TREM2, APOE, and/or MAPT genes/gene products.

### Polypeptides

APP expression and/or activity in some embodiments can be modulated by the use of MAPT polypeptides or polypeptides that can interact with MAPT (e.g., to modulate its activity).

In some embodiments, examples of polypeptides (e.g., peptides; proteins, such as enzymes; antibodies; etc.) useful in the treatment of Alzheimer's disease are provided in Salloway S P, et al. “Long-Term Follow Up of Patients with Mild-to-Moderate Alzheimer's Disease Treated with Bapineuzumab in a Phase III, Open-Label, Extension Study.” J Alzheimers Dis. (2018) 64(3):689-707; Honig L S, “Trial of Solanezumab for Mild Dementia Due to Alzheimer's Disease.” N Engl J Med. (2018) 378(4):321-330; Salloway S, et al. “A trial of gantenerumab or solanezumab in dominantly inherited Alzheimer's disease.” Nat Med. (2021) 27(7):1187-1196; Ostrowitzki S, et al. “A phase III randomized trial of gantenerumab in prodromal Alzheimer's disease.” Alzheimers Res Ther. (2017) 9(1):95; Cummings J L, et al. ABBY: A phase 2 randomized trial of crenezumab in mild to moderate Alzheimer disease. Neurology. (2018) 22; 90(21):e1889-e1897; Landen J W, et al. “Safety and pharmacology of a single intravenous dose of ponezumab in subjects with mild-to-moderate Alzheimer disease: a phase I, randomized, placebo-controlled, double-blind, dose-escalation study.” Clin Neuropharmacol. (2013) 36(1):14-23; Swanson C J, et al. “A randomized, double-blind, phase 2b proof-of-concept clinical trial in early Alzheimer's disease with lecanemab, an anti-Ap protofibril antibody.” Alzheimers Res Ther. (2021) 13(1):80; Budd Haeberlein S, et al. “Two Randomized Phase 3 Studies of Aducanumab in Early Alzheimer's Disease.” J Prev Alzheimers Dis. (2022) 9(2):197-210; U.S. Patent Application Publication No. US20190382471A1, published Dec. 19, 2019, entitled “Anti-N3pGlu amyloid beta peptide antibodies and uses thereof”; U.S. Patent Application Publication No. US20170204171A1, published Jul. 20, 2017, entitled “Anti-N3pGlu amyloid beta peptide antibodies and uses thereof”; International Patent Application Publication No. WO2012021475A2, published Feb. 16, 2012, entitled “Anti-N3pGlu amyloid beta peptide antibodies and uses thereof”; U.S. Patent Application Publication No. US20040043418A1, published Mar. 4, 2004, entitled “Humanized antibodies that sequester Abeta peptide”; U.S. Patent Application Publication No. US20100221187A1, published Sep. 2, 2010, entitled “Treatment of amyloidogenic diseases”; U.S. Patent Application Publication No. US2006257396A1, published Nov. 16, 2006, entitled “Abeta antibodies for use in improving cognition”; U.S. Pat. No. 10,040,836B2, published Aug. 7, 2018, entitled “Peptides for the treatment of neurodegenerative diseases”; U.S. Patent Application Publication No. US2003165496A1, published Sep. 4, 2003, entitled “Humanized antibodies that recognize beta amyloid peptide”; U.S. Patent Application Publication No. US20170058022A1, published Mar. 2, 2017, entitled “Camelid single-domain antibody directed against phosphorylated tau proteins and methods for producing conjugates thereof”; U.S. Patent Application Publication No. US20160347831A1, published Dec. 1, 2016, entitled “Camelid single-domain antibody directed against amyloid beta and methods for producing conjugates thereof”; U.S. Patent Application Publication No. US20150246963A1, published Sep. 3, 2015, entitled “Methods of treating Alzheimer's disease”; U.S. Patent Application Publication No. US20050169925A1, published Aug. 4, 2005, entitled “Anti-amyloid beta antibodies and their use”; U.S. Patent Application Publication No. US2016009793A1, published Jan. 14, 2016, entitled “Abeta protofibril binding antibodies”; International Patent Application Publication No. WO2021081101A1, published Apr. 29, 2021, entitled “Anti-beta-amyloid antibody for treating Alzheimer's disease”; U.S. Patent Application Publication No. US20180333487A1, published Nov. 22, 2018, entitled “Methods for treating Alzheimer's disease”; U.S. Patent Application Publication No. US20140228277A1, published Aug. 14, 2014, entitled “Peptide inhibitors of bace1”; and U.S. Patent Application Publication No. US20080248029A1, published Oct. 9, 2008, entitled “Prevention and treatment of amyloidogenic disease”; the entire contents of each of which are herein incorporated by reference.

Certain polypeptides provided in this section may be useful in treating Alzheimer's disease by modulating the activity of genes and gene products other than APP genes/gene products, such as TREM2, APOE, and/or MAPT genes/gene products.

### Small Molecules

APP expression and/or activity in some embodiments can be modulated by the use of small molecules that can modulate APP (e.g., to modulate its activity, or its expression).

In some embodiments, examples of small molecules useful in the treatment of Alzheimer's disease are provided in U.S. Patent Application Publication No. US2021261569A1, published Aug. 26, 2021, entitled “Fused heterocyclic derivatives having selective bace1 inhibitory activity”; U.S. Patent Application Publication No. US2021261561A1, published Aug. 26, 2021, entitled “Bicyclic heterocycle derivatives having selective bace1 inhibitory activity”; International Patent Application Publication No. WO2017061534A1, published Apr. 13, 2017, entitled “Dihydrothiazine derivatives”; U.S. Patent Application Publication No. US20140088111A1, published Mar. 27, 2014, entitled “Novel bicyclic pyridinones”; U.S. Pat. No. 10,022,357B2, published Jul. 17, 2018, entitled “Amyloid precursor protein MRNA blockers for treating Down syndrome and Alzheimer's disease”; U.S. Patent Application Publication No. US20140228277A1, published Aug. 14, 2014, entitled “Peptide inhibitors of bace1”; U.S. Patent Application Publication No. US20110201605A1, published Aug. 18, 2011, entitled “Heteroaryl substituted piperidines”; U.S. Patent Application Publication No. US2010093731A1, published Apr. 15, 2010, entitled “Modulators for amyloid beta”; U.S. Patent Application Publication No. US2009215759A1, published Feb. 16, 2009, entitled “Modulators for amyloid beta”; U.S. Patent Application Publication No. US2009181965A1, published Jul. 16, 2009, entitled “Modulators for amyloid beta”; U.S. Patent Application Publication No. US2008280948A1, published Nov. 13, 2008, entitled “Modulators for amyloid beta”; International Patent Application Publication No. WO2014013076A1, published Jan. 23, 2014, entitled “Hexahydropyrrolothiazine compounds”; and International Patent Application Publication No. WO2014015125A1, published Jan. 23, 2014, entitled “Fused aminodihydrothiazine derivative salts and uses thereof”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Alzheimer's disease by modulating the activity of genes and gene products other than APP genes/gene products, such as TREM2, APOE, and/or MAPT genes/gene products.

### Gene Therapies

APP expression and/or activity in some embodiments can be modulated by the use of gene therapies that can modulate APP (e.g., by delivery of nucleic acids encoding APP or other molecules that interact with APP).

In some embodiments, gene therapies, such as those involving administration of compounds encoding useful therapeutic agents, useful in the treatment of Alzheimer's disease are provided in U.S. Patent Application Publication No. US2021162072AA, published Jun. 3, 2021, entitled “Modified adeno-associated virus vectors and delivery thereof into the central nervous system”; the entire contents of which are herein incorporated by reference.

Certain gene therapies provided in this section may be useful in treating Alzheimer's disease by modulating the activity of genes and gene products other than APP genes/gene products, such as TREM2, APOE, and/or MAPT genes/gene products.

### Molecular Payloads for the Treatment of Frontotemporal Dementia (FTD)

Various molecular payloads may be useful in the treatment of frontotemporal dementia (FTD), including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of FTD may include, in some embodiments, molecular payloads which modulate (e.g., increase or decrease) expression or activity of GRN, C9orf72, MAPT, PIKFYVE, SYF2, and/or UNC13A.

Examples of oligonucleotides useful for the treatment of FTD, e.g., oligonucleotides targeting (e.g., directly or indirectly modulating the expression or activity of) genes associated with FTD (e.g., GRN, C9orf72, MAPT, PIKFYVE, SYF2, UNC13A, etc.), include those listed in Table 13 below. Each oligonucleotide provided in Table 13 may have any modification pattern disclosed herein.

TABLE 13
Oligonucleotides for the treatment of FTD

| SEQ | | Oligonucleotide | |
|---|---|---|---|
| ID | Target | Sequence | Optional Oligonucleotide Structure* |
| NO: | Gene | (5′-3′) | (5′-3′) |
| 1018 | GRN | GTCCAGGGAGAATTTGGT | es eo mes mes es es es es es eo es |
| | | | es es es es es es e, wherein: m=5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1019 | GRN | GTGGATAGGGAAAAGCAC | es eo es es es es es es es eo es es |
| | | | es es es mes es me, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1020 | GRN | GGGTCCACTGAAACGGGG | es eo es es mes mes es mes es eo es |
| | | | es es mes es es es e, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1021 | GRN | GGATAGGGAAAAGCACCT | es eo es es es es es es es eo es es |
| | | | es mes es mes mes e, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1022 | GRN | GGTCCAGGGAGAATTTGG | es eo es mes mes es es es es eo es |
| | | | es es es es es es e, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1023 | GRN | AAACGGGGAGGGGATGGC | es eo es mes es es es es es eo es es |
| | | | es es es es es me, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1024 | GRN | GAAACGGGGAGGGGATGG | es eo es es mes es es es es eo es es |
| | | | es es es es es e, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1025 | GRN | TGAAACGGGGAGGGGATG | es eo es es es mes es es es eo es es |
| | | | es es es es es e, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1026 | GRN | CTGAAACGGGGAGGGGAT | mes eo es es es es mes es es eo es |
| | | | es es es es es es e, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1027 | GRN | ACTGAAACGGGGAGGGGA | es meo es es es es es mes es eo es |
| | | | es es es es es es e, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1028 | GRN | CACTGAAACGGGGAGGGG | mes eo mes es es es es es mes eo es |
| | | | es es es es es es e, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 1029 | GRN | GGTCCACTGAAACGGGGA | es eo es mes mes es mes es es eo es |
| | | | es mes es es es es e, wherein: m = 5- |
| | | | methyl modified nucleobase, e = 2′-MOE |
| | | | sugar moiety, s = phosphorothioate |
| | | | internucleoside linkage, |
| | | | o = phosphodiester internucleoside |
| | | | linkage |
| 876 | PIKFYVE | ATGGCATGATCCCCATAAG | eseoeoeoeodsdsdsdsdsdsdsdsdsdseoeoes |
| | | C | ese |
| 877 | PIKFYVE | AGGACCAGTACAACCTGTA | eseoeoeoeodsdsdsdsdsdsdsdsdsdseoeoes |
| | | G | ese |
| 878 | PIKFYVE | AGGTGCCCCAACTTGTTAC | eseoeoeoeodsdsdsdsdsdsdsdsdsdseoeoes |
| | | C | ese |
| 879 | PIKFYVE | ACTAAGGTGCCCCAACTTG | eseoeoeoeodsdsdsdsdsdsdsdsdsdseoeoes |
| | | T | ese |
| 880 | PIKFYVE | ACCAGGTAGTCCCAAATAA | eseoeoeoeodsdsdsdsdsdsdsdsdsdseoeoes |
| | | C | ese |
| 881 | PIKFYVE | GCCTGACCCCTATACTTGA | eseoeoeoeodsdsdsdsdsdsdsdsdsdseoeoes |
| | | C | ese |
| 882 | PIKFYVE | CTGACCCCTATACTTGACA | eseoeoeoeodsdsdsdsdsdsdsdsdsdseoeoes |
| | | G | ese |
| 883 | PIKFYVE | TTAAGCCCTTTGGTCCAAC | eseoeoeoeodsdsdsdsdsdsdsdsdsdseoeoes |
| | | C | ese |
| 884 | PIKFYVE | GAGTTAGCACCCTCTACTA | eseoeoeoeodsdsdsdsdsdsdsdsdsdseoeoes |
| | | C | ese |
| 885 | PIKFYVE | GTCATAAGTCCTTGGTCAA | eseoeoeoeodsdsdsdsdsdsdsdsdsdseoeoes |
| | | C | ese |
| 886 | PIKFYVE | AAGCCTGACCCCTATACTT | eseoeoeoeodsdsdsdsdsdsdsdsdsdseoeoes |
| | | G | ese |
| 887 | PIKFYVE | AGCCTGACCCCTATACTTG | eseoeoeoeodsdsdsdsdsdsdsdsdsdseoeoes |
| | | A | ese |
| 888 | PIKFYVE | CATCCTATTAGCTTAAACC | eseoeoeoeodsdsdsdsdsdsdsdsdsdseoeoes |
| | | C | ese |
| 889 | PIKFYVE | CCTGACCCCTATACTTGAC | eseoeoeoeodsdsdsdsdsdsdsdsdsdseoeoes |
| | | A | ese |
| 890 | PIKFYVE | TATGATCTGATAGCCAACI | eseoeoeoeodsdsdsdsdsdsdsdsdsdseoeoes |
| | | C | ese |
| 891 | PIKFYVE | GTACCAGGACCAGTACAAC | eseoeoeoeodsdsdsdsdsdsdsdsdsdseoeoes |
| | | C | ese |
| 892 | PIKFYVE | CAGGACCAGTACAACCTGT | eseoeoeoeodsdsdsdsdsdsdsdsdsdseoeoes |
| | | A | ese |
| 893 | PIKFYVE | ACACCCTTTGGAGTGTCIA | eseoeoeoeodsdsdsdsdsdsdsdsdsdseoeoes |
| | | G | ese |
| 894 | PIKFYVE | GCCCCAACTTGTTACCTAA | eseoeoeoeodsdsdsdsdsdsdsdsdsdseoeoes |
| | | G | ese |
| 895 | PIKFYVE | CCCCAATCTAGTTCAAGCA | eseoeoeoeodsdsdsdsdsdsdsdsdsdseoeoes |
| | | T | ese |
| 896 | PIKFYVE | TCCAAGAGTTAGCACCCIC | eseoeoeoeodsdsdsdsdsdsdsdsdsdseoeoes |
| | | T | ese |
| 897 | PIKFYVE | GGACCAGTACAACCTGTAG | eseoeoeoeodsdsdsdsdsdsdsdsdsdseoeoes |
| | | T | ese |
| 898 | PIKFYVE | AAACCCAGTGTAGCTCCAT | eseoeoeoeodsdsdsdsdsdsdsdsdsdseoeoes |
| | | T | ese |
| 899 | PIKFYVE | TGCCCCAACTTGTTACCTA | eseoeoeoeodsdsdsdsdsdsdsdsdsdseoeoes |
| | | A | ese |
| 900 | PIKFYVE | CCAATAGCAAGCAGCCTAT | eseoeoeoeodsdsdsdsdsdsdsdsdsdseoeoes |
| | | A | ese |
| 901 | PIKFYVE | CTACAATCAAATCCTGGTA | eseoeoeoeodsdsdsdsdsdsdsdsdsdseoeoes |
| | | G | ese |
| 902 | PIKFYVE | GACCAAGTTTATGGACCCC | eseoeoeoeodsdsdsdsdsdsdsdsdsdseoeoes |
| | | A | ese |
| 903 | PIKFYVE | GTGACCACCATCTATAGTT | eseoeoeoeodsdsdsdsdsdsdsdsdsdseoeoes |
| | | A | ese |
| 904 | PIKFYVE | CTAAGGTAACTGTTCCTAG | eseoeoeoeodsdsdsdsdsdsdsdsdsdseoeoes |
| | | A | ese |
| 905 | PIKFYVE | TGACCAAGTTTATGGACCC | eseoeoeoeodsdsdsdsdsdsdsdsdsdseoeoes |
| | | C | ese |
| 906 | PIKFYVE | ACCAAGTTTATGGACCCCA | eseoeoeoeodsdsdsdsdsdsdsdsdsdseoeoes |
| | | A | ese |
| 907 | PIKFYVE | GCAATAAAGCTAACCACAT | eseoeoeoeodsdsdsdsdsdsdsdsdsdseoeoes |
| | | C | ese |
| 908 | PIKFYVE | GATTCTACCACACAGTACA | eseoeoeoeodsdsdsdsdsdsdsdsdsdseoeoes |
| | | C | ese |
| 909 | UNC13A | GCAGACAGGTGTACTGGA | esesesesesesesesesesesesesesesesese |
| 910 | UNC13A | AGACAGGTGTACTGGACA | esesesesesesesesesesesesesesesesese |
| 911 | UNC13A | ACAGGTGTACTGGACATG | esesesesesesesesesesesesesesesesese |
| 912 | UNC13A | AGGTGTACTGGACATGGT | esesesesesesesesesesesesesesesesese |
| 913 | UNC13A | AGGCAGCTGGAAGAGACA | esesesesesesesesesesesesesesesesese |
| 914 | UNC13A | GGCAGCTGGAAGAGACAT | esesesesesesesesesesesesesesesesese |
| 915 | UNC13A | GCAGCTGGAAGAGACATA | esesesesesesesesesesesesesesesesese |
| 916 | UNC13A | CAGCTGGAAGAGACATAC | esesesesesesesesesesesesesesesesese |
| 917 | UNC13A | AGCTGGAAGAGACATACC | esesesesesesesesesesesesesesesesese |
| 918 | UNC13A | GCTGGAAGAGACATACCC | esesesesesesesesesesesesesesesesese |
| 919 | UNC13A | CTGGAAGAGACATACCCA | esesesesesesesesesesesesesesesesese |
| 920 | UNC13A | TGGAAGAGACATACCCAG | esesesesesesesesesesesesesesesesese |
| 921 | UNC13A | GGAAGAGACATACCCAGA | esesesesesesesesesesesesesesesesese |
| 922 | UNC13A | GAAGAGACATACCCAGAC | esesesesesesesesesesesesesesesesese |
| 923 | UNC13A | AAGAGACATACCCAGACA | esesesesesesesesesesesesesesesesese |
| 924 | UNC13A | AGAGACATACCCAGACAC | esesesesesesesesesesesesesesesesese |
| 925 | UNC13A | GAGACATACCCAGACACA | esesesesesesesesesesesesesesesesese |
| 926 | UNC13A | AGACATACCCAGACACAA | esesesesesesesesesesesesesesesesese |
| 927 | UNC13A | GACATACCCAGACACAAA | esesesesesesesesesesesesesesesesese |
| 928 | UNC13A | ACATACCCAGACACAAAC | esesesesesesesesesesesesesesesesese |
| 929 | UNC13A | CATACCCAGACACAAACG | esesesesesesesesesesesesesesesesese |
| 930 | UNC13A | TAGTTCCTGGGGATAAGA | esesesesesesesesesesesesesesesesese |
| 931 | UNC13A | AGTTCCTGGGGATAAGAG | esesesesesesesesesesesesesesesesese |
| 932 | UNC13A | GTTCCTGGGGATAAGAGT | esesesesesesesesesesesesesesesesese |
| 933 | UNC13A | TTCCTGGGGATAAGAGTT | esesesesesesesesesesesesesesesesese |
| 934 | UNC13A | TCCTGGGGATAAGAGTTC | esesesesesesesesesesesesesesesesese |
| 935 | UNC13A | CCTGGGGATAAGAGTTCT | esesesesesesesesesesesesesesesesese |
| 936 | UNC13A | CTGGGGATAAGAGTTCTT | esesesesesesesesesesesesesesesesese |
| 937 | UNC13A | TGGGGATAAGAGTTCTTT | esesesesesesesesesesesesesesesesese |
| 938 | UNC13A | GGGGATAAGAGTTCTTTC | esesesesesesesesesesesesesesesesese |
| 939 | UNC13A | GGGATAAGAGTTCTTTCC | esesesesesesesesesesesesesesesesese |
| 940 | UNC13A | CATCTGTTCAATCATTCA | esesesesesesesesesesesesesesesesese |
| 941 | UNC13A | TCTGTTCAATCATTCATT | esesesesesesesesesesesesesesesesese |
| 942 | UNC13A | TGTTCAATCATTCATTCA | esesesesesesesesesesesesesesesesese |
| 943 | UNC13A | TTCAATCATTCATTCATT | esesesesesesesesesesesesesesesesese |
| 944 | UNC13A | CAATCATTCATTCATTCA | esesesesesesesesesesesesesesesesese |
| 945 | UNC13A | ATCATTCATTCATTCACC | esesesesesesesesesesesesesesesesese |
| 946 | UNC13A | CATTCATTCATTCACCAG | esesesesesesesesesesesesesesesesese |
| 947 | UNC13A | TTCATTCATTCACCAGCA | esesesesesesesesesesesesesesesesese |
| 948 | UNC13A | CATTCATTCACCAGCATT | esesesesesesesesesesesesesesesesese |
| 949 | UNC13A | TTCATTCACCAGCATTTA | esesesesesesesesesesesesesesesesese |
| 950 | UNC13A | CATTCACCAGCATTTATT | esesesesesesesesesesesesesesesesese |
| 951 | UNC13A | TTCACCAGCATTTATTCA | esesesesesesesesesesesesesesesesese |
| 952 | UNC13A | CACCAGCATTTATTCAAC | esesesesesesesesesesesesesesesesese |
| 953 | UNC13A | CCAGCATTTATTCAACAA | esesesesesesesesesesesesesesesesese |
| 954 | UNC13A | AGCATTTATTCAACAAAC | esesesesesesesesesesesesesesesesese |
| 955 | UNC13A | CATTTATTCAACAAACTA | esesesesesesesesesesesesesesesesese |
| 956 | UNC13A | TTTATTCAACAAACTAGT | esesesesesesesesesesesesesesesesese |
| 957 | UNC13A | TATTCAACAAACTAGTTC | esesesesesesesesesesesesesesesesese |
| 958 | UNC13A | TTCAACAAACTAGTTCCT | esesesesesesesesesesesesesesesesese |
| 959 | UNC13A | CAACAAACTAGTTCCTGG | esesesesesesesesesesesesesesesesese |
| 960 | UNC13A | ACAAACTAGTTCCTGGGG | esesesesesesesesesesesesesesesesese |
| 961 | UNC13A | AAACTAGTTCCTGGGGAT | esesesesesesesesesesesesesesesesese |
| 962 | UNC13A | ACTAGTTCCTGGGGATAA | esesesesesesesesesesesesesesesesese |
| 963 | UNC13A | GGATAAGAGTTCTTTCCA | esesesesesesesesesesesesesesesesese |
| 964 | UNC13A | GATAAGAGTTCTTTCCAG | esesesesesesesesesesesesesesesesese |
| 965 | UNC13A | ATAAGAGTTCTTTCCAGG | esesesesesesesesesesesesesesesesese |
| 966 | UNC13A | TAAGAGTTCTTTCCAGGA | esesesesesesesesesesesesesesesesese |
| 967 | UNC13A | AAGAGTTCTTTCCAGGAA | esesesesesesesesesesesesesesesesese |
| 968 | UNC13A | AGAGTTCTTTCCAGGAAA | esesesesesesesesesesesesesesesesese |
| 969 | UNC13A | GAGTTCTTTCCAGGAAAC | esesesesesesesesesesesesesesesesese |
| 970 | UNC13A | AGTTCTTTCCAGGAAACC | esesesesesesesesesesesesesesesesese |
| 971 | UNC13A | GTTCTTTCCAGGAAACCC | esesesesesesesesesesesesesesesesese |
| 972 | UNC13A | ACTCATCACTCATTCATC | esesesesesesesesesesesesesesesesese |
| 973 | UNC13A | TCATCACTCATTCATCTG | esesesesesesesesesesesesesesesesese |
| 974 | UNC13A | ATCACTCATTCATCTGTT | esesesesesesesesesesesesesesesesese |
| 975 | UNC13A | CACTCATTCATCTGTTCA | esesesesesesesesesesesesesesesesese |
| 976 | UNC13A | CTCATTCATCTGTTCAAT | esesesesesesesesesesesesesesesesese |
| 977 | UNC13A | CATTCATCTGTTCAATCA | esesesesesesesesesesesesesesesesese |
| 978 | UNC13A | TTCATCTGTTCAATCATT | esesesesesesesesesesesesesesesesese |
| 979 | UNC13A | CACCCATCTCTCCATCCAT | LsdsdsLsdsdsLsdsdsLsdsdsLsdsdsLsdsds |
| | | GC | Lsdsd |
| 980 | UNC13A | CACCCATCTCTCCATCCAT | LsdsdsLsdsdsLsdsdsLsdsdsLsdsdsLsdsds |
| | | CC | Lsdsd |
| 981 | UNC13A | CTCACCCATCTCTCCATCC | LsdsdsLsdsdsLsdsdsLsdsdsLsdsdsLsdsds |
| | | AT | LsdsL |
| 982 | UNC13A | TGTACTCACCCATCTCTCC | LsdsdsLsdsdsLsdsdsLsdsdsLsdsdsLsdsds |
| | | AT | LsdsL |
| 983 | UNC13A | TCCATGTACTCACCCATCT | LsdsdsLsdsdsLsdsdsLsdsdsLsdsdsLsdsds |
| | | CT | LsdsL |
| 984 | UNC13A | TCCATCCATGTACTCACCC | LsdsdsLsdsdsLsdsdsLsdsdsLsdsdsLsdsds |
| | | AT | LsdsL |
| 985 | UNC13A | ATCTATCCATCCATGTACT | LsdsdsLsdsdsLsdsdsLsdsdsLsdsdsLsdsds |
| | | CA | LsdsL |
| 986 | UNC13A | TCACCCATCTCTCCATC | LsdsdsLsdsdsLsdsdsLsdsdsLsdsdsLsL |
| 986 | UNC13A | TCACCCATCTCTCCATC | LsdsLsdsLsdsLsdsLsdsLsdsLsdsLSdsL |
| 987 | UNC13A | GTACTCACCCATCTCTC | LsdsdsLsdsdsLsdsdsLsdsdsLsdsdsLsL |
| 987 | UNC13A | GTACTCACCCATCTCTC | LsdsLsdsLsdsLsdsLsdsLsdsLsdsLsdsL |
| 988 | UNC13A | CACCCATCTCTCC | dsLsdsLsdsLsdsLsdsLsdsLsd |
| 989 | UNC13A | CAUGUACUCACCCAUCUCU | msmsmsmsmsmsmsmsmsmsmsmsmsmsmsmsmsms |
| | | CCAUCC | msmsmsmsmsmsm |
| 990 | UNC13A | AUCCAUGUACUCACCCAUC | msmsmsmsmsmsmsmsmsmsmsmsmsmsmsmsmsms |
| | | UCUCCAUCCAU | msmsmsmsmsmsmsmsmsmsmsm |
| 991 | UNC13A | CAGGCAGCTGGAAGAGACA | LsdsdsLsdsdsLsdsdsLsdsdsLsdsdsLsdsds |
| | | TA | LsdsL |
| 992 | UNC13A | AACCCAGGCAGCTGGAAGA | LsdsdsLsdsdsLsdsdsLsdsdsLsdsdsLsdsds |
| | | GA | LsdsL |
| 993 | UNC13A | GGAAACCCAGGCAGCTGGA | LsdsdsLsdsdsLsdsdsLsdsdsLsdsdsLsdsds |
| | | AG | LsdsL |
| 994 | UNC13A | TAGGGCTGGAATAGAAGGA | LsdsdsLsdsdsLsdsdsLsdsdsLsdsdsLsdsds |
| | | AG | LsdsL |
| 995 | UNC13A | TGAGTGGTTAGGGCTGGAA | LsdsdsLsdsdsLsdsdsLsdsdsLsdsdsLsdsds |
| | | TA | LsdsL |

*Unless otherwise noted,
e = 2′-MOE modified sugar; L = locked nucleic acid (LNA) nucleoside; d = deoxyribonucleoside; m = 2′-O-methyl ribonucleoside; s = phosphorothioate internucleoside linkage.
In each sequence listed in Table 6, each T may be optionally and independently replaced with a U, and each U may be optionally and independently replaced with a T.

Examples of small molecules useful for the treatment of FTD include:

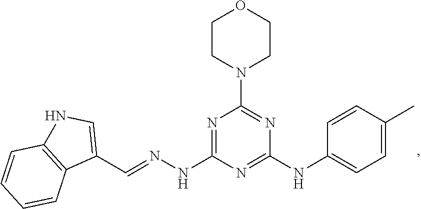 [SMILES: Cc1ccc(Nc2nc(N/N=C/c3c[nH]c4ccccc34)nc(N3CCOCC3)n2)cc1]

apilimod, APY0201, YM-201636, and pharmaceutically acceptable salts, co-crystals, tautomers, stereoisomers, solvates, hydrates, polymorphs, isotopically enriched compounds, and prodrugs thereof.

Examples of polypeptides useful for the treatment of FTD include antibodies, proteins, peptides, and enzymes. In some embodiments, a polypeptide useful for the treatment of FTD comprises a progranulin protein or functional fragment thereof, a PIPKIII protein or functional fragment thereof, a pre-mRNA-splicing factor SYF2 protein or functional fragment thereof, or an unc-13 homolog A protein or functional fragment thereof.

### Molecular Payloads Targeting GRN

The GRN gene, which encodes progranulin protein, and mutations therein, are implicated in FTD. Modulation of GRN expression and activity (e.g., by suppressing the expression and/or activity of mutant progranulin protein and/or its interactions with other proteins) therefore in some embodiments can have a therapeutic effect in subjects with FTD.

### Oligonucleotides

GRN (and/or progranulin protein encoded by GRN) expression and/or activity in some embodiments can be modulated by the use of oligonucleotides targeting GRN sequences.

In some embodiments, an oligonucleotide useful for the treatment of FTD, e.g., targeting (e.g., directly or indirectly modulating the expression or activity of) GRN, comprises a region of complementarity to a GRN transcript provided in Table 3, e.g., provided by SEQ ID NO: 837.

In some embodiments, examples of oligonucleotides useful for the treatment of FTD, e.g., targeting (e.g., directly or indirectly modulating the expression or activity of) GRN, are provided in WO2023092057A1, published May 25, 2023, entitled “Compounds and methods for modulating progranulin expression”; the entire contents of which are herein incorporated by reference.

Certain oligonucleotides provided in this section may be useful in treating FTD by modulating the activity of genes and/or gene products other than GRN genes/gene products, such as other genes/gene products associated with FTD.

### Polypeptides

GRN expression and/or activity in some embodiments can be modulated by the use of progranulin polypeptides or polypeptides that can interact with progranulin (e.g., to modulate its biological activity and/or its interaction with other biomolecules).

In some embodiments, examples of polypeptides (e.g., peptides; proteins, such as enzymes; antibodies; etc.) useful in the treatment of FTD include progranulin protein and functional fragments thereof.

Certain polypeptides provided in this section may be useful in treating FTD by modulating the activity of genes and gene products other than GRN genes/gene products, such as other genes/gene products associated with FTD.

### Small Molecules

GRN expression and/or activity in some embodiments can be modulated by the use of small molecules that can modulate progranulin (e.g., to modulate its biological activity, its expression, and/or its interaction with other biomolecules).

In some embodiments, examples of small molecules useful in the treatment of FTD are small molecules that increase or decrease expression of GRN, and/or that increase or decrease progranulin protein levels or activity. In some embodiments, small molecules useful in the treatment of FTD are provided in WO2021194607A1, published Sep. 30, 2021, entitled “Methods of using rho kinase inhibitors to treat frontotemporal dementia”; US20140179678A1, published Jun. 26, 2014, entitled “Methods of targeted treatment of frontotemporal lobar degeneration”; the entire contents of each of which are herein incorporated by reference.

Certain small molecules provided in this section may be useful in treating FTD by modulating the activity of genes and gene products other than GRN genes/gene products, such as other genes/gene products associated with FTD.

### Gene Therapies

GRN expression and/or activity in some embodiments can be modulated by the use of gene therapies that can modulate GRN (e.g., by delivery of nucleic acids encoding GRN or other molecules that interact with GRN).

In some embodiments, gene therapies, such as those involving administration of a compounds encoding useful therapeutic agents, useful in the treatment of FTD are provided in US20200332265A1, published Oct. 22, 2020, entitled “Gene therapies for lysosomal disorders”; US20220136008A1, published May 5, 2022, entitled “Recombinant adeno-associated virus for treatment of grn-associated adult-onset neurodegeneration”; US20200231954A1, published Jul. 23, 2020, entitled “Gene therapies for lysosomal disorders”; US20190328906A1, published Oct. 31, 2019, entitled “Therapy for frontotemporal dementia”; the entire contents of each of which are herein incorporated by reference.

Certain gene therapies provided in this section may be useful in treating FTD by modulating the activity of genes and gene products other than GRN genes/gene products, such as other genes/gene products associated with FTD.

### Molecular Payloads Targeting C9orf72

The C9orf72 gene, which encodes the chromosome 9 open reading frame 72 protein, and mutations therein, are implicated in FTD, which predominantly affects neurons in the frontal and temporal lobes of the brain. Modulation of C9orf72 expression and activity (e.g., by suppressing the expression of mutant C9orf72 and/or activity of the protein encoded thereby) therefore in some embodiments can have a therapeutic effect in subjects with FTD.

### Oligonucleotides

C9orf72 expression and/or activity in some embodiments can be modulated by the use of oligonucleotides targeting C9orf72 sequences.

In some embodiments, an oligonucleotide useful for the treatment of FTD, e.g., targeting (e.g., directly or indirectly modulating the expression or activity of) C9orf72, comprises a region of complementarity to a C0orf72 transcript provided in Table 3, e.g., provided by any one of SEQ ID NOs: 393-395.

In some embodiments, examples of oligonucleotides useful for the treatment of FTD, e.g., targeting (e.g., directly or indirectly modulating the expression or activity of) C9orf72, are provided in U.S. Pat. No. 10,577,604B2, published Mar. 3, 2020, entitled “Methods for monitoring C9ORF72 expression”; U.S. Pat. No. 10,443,052B2, published Oct. 15, 2019, entitled “Compositions for modulating C9ORF72 expression”; U.S. Pat. No. 10,793,855B2, published Oct. 6, 2020, entitled “Compositions for modulating expression of C9ORF72 antisense transcript”; U.S. Pat. No. 10,815,483B2, published Oct. 27, 2020, entitled “Compositions for modulating C9ORF72 expression”; U.S. Pat. No. 11,260,073B2, published Mar. 1, 2022, entitled “Compositions and methods for modulating C9ORF72”; U.S. Pat. No. 10,407,678B2, published Oct. 9, 2019, entitled “Compositions for modulating expression of C9ORF72 antisense transcript”; U.S. Pat. No. 11,162,096B2, published Nov. 2, 2021, entitled “Compositions for modulating expression of C9ORF72 antisense transcript”; U.S. Pat. No. 10,066,228B2, published Sep. 4, 2018, entitled “Oligonucleotides for treating expanded repeat diseases”; U.S. Pat. No. 11,345,915B2, published May 31, 2022, entitled “RNA modulating oligonucleotides with improved characteristics for the treatment of neuromuscular disorders”; U.S. Pat. No. 9,963,699B2, published May 8, 2018, entitled “Methods for modulating C9ORF72 expression”; U.S. Pat. No. 10,221,414B2, published Mar. 5, 2019, entitled “Compositions for modulating C9ORF72 expression”; US Patent Application Publication No. 20160108396A1, published Apr. 21, 2016, entitled “Oligomers targeting hexanucleotide repeat expansion in human C9ORF72 gene”; U.S. Pat. No. 10,538,762, published Jan. 21, 2020, entitled “Allele selective inhibition of mutant C9orf72 foci expression by duplex RNAS targeting the expanded hexanucleotide repeat”; U.S. Pat. No. 10,597,660B2, published Mar. 24, 2020, entitled “Compositions and methods of treating amyotrophic lateral sclerosis (ALS)”; U.S. Pat. No. 11,118,179B2, published Sep. 14, 2021, entitled “Mixed tricyclo-DNA, 2′-modified RNA oligonucleotide compositions and uses thereof”; US Patent Application Publication No. 20210284629A1, published Sep. 16, 2021, entitled “Methods and compounds for the treatment of genetic disease”; US Patent Application Publication No. 20210269825A1, published Sep. 2, 2021, entitled “Compositions and methods for reducing spliceopathy and treating rna dominance disorders”; US Patent Application Publication No. 20200385737A1, published Dec. 10, 2020, entitled “OLIGONUCLEOTIDE-BASED MODULATION OF C9orf72”; US Patent Application Publication No. 20200385723A1, published Dec. 10, 2020, entitled “Anti-c9orf72 oligonucleotides and related methods”; US Patent Application Publication No. 20220145300A1, published May 12, 2022, entitled “Oligonucleotide compositions and methods of use thereof”; US Patent Application Publication No. 20210032620A1, published Feb. 4, 2021, entitled “Oligonucleotide compositions and methods thereof”; International PCT Application Publication No. WO2021119226A1, published Dec. 10, 2020, entitled “Human chromosome 9 open reading frame 72 (c9orf72) irna agent compositions and methods of use thereof”; US Patent Application Publication No. 20210340535A1, published Nov. 4, 2021, entitled “DUAL-ACTING siRNA BASED MODULATION OF C9orf72”; International PCT Application Publication No. WO2021205005A2, published Oct. 14, 2021, entitled “Antisense sequences for treating amyotrophic lateral sclerosis”; the entire contents of each of which are herein incorporated by reference.

Certain oligonucleotides provided in this section may be useful in treating FTD by modulating the activity of genes and/or gene products other than C9orf72 genes/gene products, such as GRN, MAPT, PIKFYVE, SYF2, and/or UNC13A genes/gene products.

### Polypeptides

C9orf72 expression and/or activity of the protein encoded thereby in some embodiments can be modulated by the use of polypeptides, such as polypeptides that can interact with C9orf72 and/or its encoded protein (e.g., to modulate its biological activity and/or its interaction with other biomolecules).

In some embodiments, examples of polypeptides (e.g., peptides; proteins, such as enzymes; antibodies; etc.) useful in the treatment of FTD are provided in U.S. Pat. No. 10,295,547B2, published May 21, 2019, entitled “Use and treatment of di-amino acid repeat-containing proteins associated with ALS”; U.S. Pat. No. 11,197,911B2, published Dec. 14, 2021, entitled “Peptidylic inhibitors targeting C9ORF72 hexanucleotide repeat-mediated neurodegeneration”; US Patent Application Publication No. 20220153874A1, published May 19, 2022, entitled “Human-derived anti-(poly-ga) dipeptide repeat (dpr) antibody”; U.S. Pat. No. 9,329,182B2, published May 3, 2016, entitled “Method of treating motor neuron disease with an antibody that agonizes MuSK”; the entire contents of each of which are herein incorporated by reference.

Certain polypeptides provided in this section may be useful in treating FTD by modulating the activity of genes and gene products other than C9orf72 genes/gene products, such as GRN, MAPT, PIKFYVE, SYF2, and/or UNC13A genes/gene products.

### Small Molecules

C9orf72 expression and/or the activity of the protein it encodes in some embodiments can be modulated by the use of small molecules that can modulate C9orf72 (e.g., to modulate its biological activity, its expression, and/or its interaction with other biomolecules).

In some embodiments, examples of small molecules useful in the treatment of FTD are provided in U.S. Pat. No. 10,675,293B2, published Jun. 9, 2020, entitled “Nucleoside agents for the reduction of the deleterious activity of extended nucleotide repeat containing genes”; International PCT Application Publication No. WO2021174167A1, published Sep. 2, 2021, entitled “Compounds and methods for modulating splicing”; U.S. Pat. No. 11,241,417B2, published Feb. 8, 2022, entitled “Compositions and methods for the treatment and prevention of neurological disorders”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating FTD by modulating the activity of genes and gene products other than C9orf72 genes/gene products, such as GRN, MAPT, PIKFYVE, SYF2, and/or UNC13A genes/gene products.

### Gene Therapies

C9orf72 expression and/or activity in some embodiments can be modulated by the use of gene therapies that can modulate C9orf72 (e.g., by delivery of nucleic acids encoding C9orf72 or other molecules that interact with the protein it encodes).

In some embodiments, gene therapies, such as those involving administration of a compounds encoding useful therapeutic agents, useful in the treatment of FTD are provided in U.S. Pat. No. 10,597,660B2, published Mar. 24, 2020, entitled “Compositions and methods of treating amyotrophic lateral sclerosis (ALS)”; US Patent Application Publication No. 20210269825A1, published Sep. 2, 2021, entitled “Compositions and methods for reducing spliceopathy and treating ma dominance disorders”; U.S. Pat. No. 10,801,027B2, published Oct. 13, 2020, entitled “Inhibitors of SRSF1 to treat neurodegenerative disorders”; International PCT Application Publication No. WO2021160464A1, published Aug. 19, 2021, entitled “Gene therapy”; the entire contents of each of which are herein incorporated by reference.

Certain gene therapies provided in this section may be useful in treating FTD by modulating the activity of genes and gene products other than C9orf72 genes/gene products, such as GRN, MAPT, PIKFYVE, SYF2, and/or UNC13A genes/gene products.

### Molecular Payloads Targeting MAPT

The MAPT gene, and mutations therein, are implicated in FTD, which predominantly affects neurons in the frontal and temporal lobes of the brain. Modulation of MAPT expression and activity (e.g., by suppressing the expression of MAPT or a mutant form thereof and/or activity of a wild-type or mutant form of a protein encoded by MAPT) therefore in some embodiments can have a therapeutic effect in subjects with FTD.

### Oligonucleotides

MAPT expression and/or activity in some embodiments can be modulated by the use of oligonucleotides targeting MAPT sequences.

In some embodiments, an oligonucleotide useful for the treatment of FTD, e.g., targeting (e.g., directly or indirectly modulating the expression or activity of) MAPT, comprises a region of complementarity to a MAPT transcript provided in Table 3, e.g., provided by any one of SEQ ID NOs: 506-534.

In some embodiments, examples of oligonucleotides useful for the treatment of FTD, e.g., targeting (e.g., directly or indirectly modulating the expression or activity of) MAPT, are provided in International Patent Application Publication No. WO2022077024A1, published Apr. 14, 2022, entitled “Selective delivery of oligonucleotides to glial cells”; International Patent Application Publication No. WO2022009987A1, published Jan. 13, 2022, entitled “Method for treating Alzheimer's disease by targeting mapt gene”; International Patent Application Publication No. WO2021202511A2, published Oct. 7, 2021, entitled “Microtubule associated protein tau (MAPT) iRNA agent compositions and methods of use thereof”; U.S. Pat. No. 11,053,498B2, published Jul. 6, 2021, entitled “Compounds and methods for reducing Tau expression”; U.S. Pat. No. 10,273,474B2, published Apr. 30, 2019, entitled “Methods for modulating Tau expression for reducing seizure and modifying a neurodegenerative syndrome”; U.S. Patent Application Publication No. US20180161356A1, published Jun. 14, 2018, entitled “Tau antisense oligomers and uses thereof”; U.S. Patent Application Publication No. US2021363523A1, published Nov. 25, 2021, entitled “Oligonucleotides for mapt modulation”; International Patent Application Publication No. WO2021178237A2, published Sep. 10, 2021, entitled “Oligonucleotide compositions and methods thereof”; U.S. Patent Application Publication No. US20200010831A1, published Mar. 22, 2022, entitled “Oligonucleotides for modulating tau expression”; U.S. Patent Application Publication No. US2018066254A1, published Dec. 24, 2019, entitled “Rna interference mediated therapy for neurodegenerative diseases”; U.S. Patent Application Publication No. US2016237427A1, published Oct. 13, 2020, entitled “Tau Antisense Oligomers and Uses Thereof”; U.S. Pat. No. 11,332,733B2, published May 17, 2022, entitled “Modified compounds and uses thereof”; and U.S. Pat. No. 9,644,207B2, published May 9, 2017, entitled “Compositions and methods for modulating tau expression”; the entire contents of each of which are herein incorporated by reference.

Certain oligonucleotides provided in this section may be useful in treating FTD by modulating the activity of genes and/or gene products other than MAPT genes/gene products, such as GRN, C9orf72, PIKFYVE, SYF2, and/or UNC13A genes/gene products.

### Polypeptides

MAPT expression and/or activity in some embodiments can be modulated by the use of MAPT polypeptides or polypeptides that can interact with MAPT (e.g., to modulate its activity).

In some embodiments, examples of polypeptides (e.g., peptides; proteins, such as enzymes; antibodies; etc.) useful in the treatment of FTD are provided in International Patent Application Publication No. WO2021151012A1, published Jul. 29, 2021, entitled “Zinc finger protein transcription factors for repressing tau expression”; U.S. Patent Application Publication No. US20220146535A1, published May 12, 2022, entitled “Compounds and methods targeting human tau”; U.S. Patent Application Publication No. US20220127345A1, published Apr. 28, 2022, entitled “Methods of Reducing Tau in Human Subjects”; U.S. Patent Application Publication No. US20210163582A1, published Oct. 19, 2021, entitled “Antibodies that bind to pathological tau species and uses thereof”; International Patent Application Publication No. WO2021262791A1, published Dec. 30, 2021, entitled “High affinity antibodies targeting tau phosphorylated at serine 413”; U.S. Patent Application Publication No. US20200216522A1, published Jul. 9, 2020, entitled “Anti-tau antibodies and methods of use thereof”; U.S. Patent Application Publication No. US20200131255A1, published Apr. 30, 2020, entitled “Methods of treating neurodegenerative diseases”; U.S. Patent Application Publication No. US20190135905A1, published May 9, 2019, entitled “Compositions and methods for treating tauopathies”; U.S. Patent Application Publication No. US2018194832A1, published Jul. 12, 2018, entitled “Tau-binding antibodies”; U.S. Patent Application Publication No. US20190135905A1, published May 9, 2019, entitled “Compositions and methods for treating tauopathies”; U.S. Pat. No. 10,040,836B2, published Aug. 7, 2018, entitled “Peptides for the treatment of neurodegenerative diseases”; U.S. Patent Application Publication No. US20180201666A1, published Jul. 19, 2018, entitled “Tau-binding antibodies”; U.S. Patent Application Publication No. US20190135905A1, published May 9, 2019, entitled “Compositions and methods for treating tauopathies”; U.S. Patent Application Publication No. US20180142007A1, published May 24, 2018, entitled “Humanized tau antibodies in Alzheimer's disease”; U.S. Patent Application Publication No. US20170296680A1, published Oct. 19, 2017, entitled “Anti-tau antibody and uses thereof”; U.S. Patent Application Publication No. US20170152307A1, published Jun. 1, 2017, entitled “Antibodies and antigen-binding fragments that specifically bind to microtubule-associated protein tau”; U.S. Patent Application Publication No. US20170015738A1, published Jan. 19, 2017, entitled “Antibodies Specific for Hyperphosphorylated Tau and Methods of Use Thereof”; U.S. Patent Application Publication No. US20160376351A1, published Dec. 29, 2016, entitled “Anti-tau antibodies and methods of use”; U.S. Patent Application Publication No. US20150183855A1, published Jul. 2, 2015, entitled “Antibodies to tau”; U.S. Patent Application Publication No. US20150344553A1, published Dec. 3, 2015, entitled “Human anti-tau antibodies”; and U.S. Patent Application Publication No. US20120087861A1, published Apr. 12, 2012, entitled “Human Anti-Tau Antibodies”; the entire contents of each of which are herein incorporated by reference.

Certain polypeptides provided in this section may be useful in treating FTD by modulating the activity of genes and gene products other than MAPT genes/gene products, such as GRN, C9orf72, PIKFYVE, SYF2, and/or UNC13A genes/gene products.

### Small Molecules

MAPT expression and/or activity in some embodiments can be modulated by the use of small molecules that can modulate MAPT (e.g., to modulate its activity, or its expression).

In some embodiments, examples of small molecules useful in the treatment of FTD are provided in Turner R S, et al. “Nilotinib Effects on Safety, Tolerability, and Biomarkers in Alzheimer's Disease.” Ann Neurol (2020) 88(1):183-194; Melis V, et al. “Effects of oxidized and reduced forms of methylthioninium in two transgenic mouse tauopathy models.” Behav Pharmacol. (2015) 26(4):353-68; International Patent Application Publication No. WO2022078971A1, published Apr. 21, 2022, entitled “Novel compounds”; and International Patent Application Publication No. WO2019134978A1, published Jul. 11, 2019, entitled “1, 3, 4, 5-tetrahydro-2 h-pyrido[4,3-b]indole derivatives for the treatment, alleviation or prevention of disorders associated with tau aggregates like Alzheimer's disease”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating FTD by modulating the activity of genes and gene products other than MAPT genes/gene products, such as GRN, C9orf72, PIKFYVE, SYF2, and/or UNC13A genes/gene products.

### Gene Therapies

MAPT expression and/or activity in some embodiments can be modulated by the use of gene therapies that can modulate MAPT (e.g., by delivery of nucleic acids encoding MAPT or other molecules that interact with MAPT).

In some embodiments, gene therapies, such as those involving administration of a compounds encoding useful therapeutic agents, useful in the treatment of FTD are provided in International Patent Application Publication No. WO2021151012A1, published Jul. 29, 2021, entitled “Zinc finger protein transcription factors for repressing tau expression”; the entire contents of which are herein incorporated by reference.

Certain gene therapies provided in this section may be useful in treating FTD by modulating the activity of genes and gene products other than MAPT genes/gene products, such as GRN, C9orf72, PIKFYVE, SYF2, and/or UNC13A genes/gene products.

### Molecular Payloads Targeting PIKFYVE

The PIKFYVE gene, which encodes the phosphatidylinositol-3-phosphate 5-kinase type III (PIPKIII) protein, and mutations therein, are implicated in FTD. Modulation of PIKFYVE expression and activity (e.g., by suppressing the expression and/or activity of mutant PIPKIII protein and/or its interactions with other proteins) therefore in some embodiments can have a therapeutic effect in subjects with FTD.

### Oligonucleotides

PIKFYVE (and/or PIPKIII protein encoded by PIKFYVE) expression and/or activity in some embodiments can be modulated by the use of oligonucleotides targeting PIKFYVE sequences.

In some embodiments, an oligonucleotide useful for the treatment of FTD, e.g., targeting (e.g., directly or indirectly modulating the expression or activity of) PIKFYVE, comprises a region of complementarity to a PIKFYVE transcript provided in Table 3, e.g., provided by any one of SEQ ID NOs: 143-148.

In some embodiments, examples of oligonucleotides useful for the treatment of FTD, e.g., targeting (e.g., directly or indirectly modulating the expression or activity of) PIKFYVE, are provided in US20220411804A1, published Dec. 29, 2022, entitled “Pikfyve antisense oligonucleotides”; the entire contents of which are herein incorporated by reference.

Certain oligonucleotides provided in this section may be useful in treating FTD by modulating the activity of genes and/or gene products other than PIKFYVE genes/gene products, such as other genes/gene products associated with FTD.

### Polypeptides

PIKFYVE expression and/or activity in some embodiments can be modulated by the use of PIPKIII polypeptides or polypeptides that can interact with PIPKIII (e.g., to modulate its biological activity and/or its interaction with other biomolecules).

In some embodiments, examples of polypeptides (e.g., peptides; proteins, such as enzymes; antibodies; etc.) useful in the treatment of FTD include PIPKIII protein and functional fragments thereof.

Certain polypeptides provided in this section may be useful in treating FTD by modulating the activity of genes and gene products other than PIKFYVE genes/gene products, such as other genes/gene products associated with FTD.

### Small Molecules

PIKFYVE expression and/or activity in some embodiments can be modulated by the use of small molecules that can modulate PIPKIII (e.g., to modulate its biological activity, its expression, and/or its interaction with other biomolecules).

In some embodiments, examples of small molecules useful in the treatment of FTD are provided in US20190192527A1, published Jun. 27, 2019, entitled “Compositions comprising pikfyve inhibitors and methods related to inhibition of rank signaling”; WO2017040971A1, published Mar. 9, 2017, entitled “Methods of using inhibitors of pikfyve for the treatment of lysosomal storage disorders and neurodegenerative diseases”; WO2022086993A1, published Apr. 28, 2022, entitled “Novel inhibitors of pikfyve and methods using same”; US20210139505A1, published May 13, 2021, entitled “PIKfyve Inhibitors”; U.S. Ser. No. 10/758,545B2, published Sep. 1, 2020, entitled “Methods to treat neurological diseases”; U.S. Ser. No. 11/066,410B2, published Jul. 20, 2021, entitled “Fused triazolo-pyrimidine compounds having useful pharmaceutical application”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating FTD by modulating the activity of genes and gene products other than PIKFYVE genes/gene products, such as other genes/gene products associated with FTD.

### Gene Therapies

PIKFYVE expression and/or activity in some embodiments can be modulated by the use of gene therapies that can modulate PIKFYVE (e.g., by delivery of nucleic acids encoding PIKFYVE or other molecules that interact with PIKFYVE).

In some embodiments, gene therapies, such as those involving administration of a compounds encoding useful therapeutic agents, useful in the treatment of FTD include payloads which encode PIPKIII or functional fragments thereof.

Certain gene therapies provided in this section may be useful in treating FTD by modulating the activity of genes and gene products other than PIKFYVE genes/gene products, such as other genes/gene products associated with FTD.

### Molecular Payloads Targeting SYF2

The SYF2 gene, which encodes the pre-mRNA-splicing factor SYF2 protein, and mutations therein, are implicated in FTD. Modulation of SYF2 expression and activity (e.g., by suppressing the expression and/or activity of mutant pre-mRNA-splicing factor SYF2 protein and/or its interactions with other proteins) therefore in some embodiments can have a therapeutic effect in subjects with FTD.

### Oligonucleotides

SYF2 (and/or pre-mRNA-splicing factor SYF2 protein encoded by SYF2) expression and/or activity in some embodiments can be modulated by the use of oligonucleotides targeting SYF2 sequences.

In some embodiments, an oligonucleotide useful for the treatment of FTD, e.g., targeting (e.g., directly or indirectly modulating the expression or activity of) SYF2, comprises a region of complementarity to an SYF2 transcript provided in Table 3, e.g., provided by any one of SEQ ID NOs: 167-168.

In some embodiments, examples of oligonucleotides useful for the treatment of FTD, e.g., targeting (e.g., directly or indirectly modulating the expression or activity of) SYF2, are provided in US20230066380A1, published Mar. 2, 2023, entitled “Antagonism as a therapy for tdp-43 proteinopathies”; the entire contents of which are herein incorporated by reference.

Certain oligonucleotides provided in this section may be useful in treating FTD by modulating the activity of genes and/or gene products other than SYF2 genes/gene products, such as other genes/gene products associated with FTD.

### Polypeptides

SYF2 expression and/or activity in some embodiments can be modulated by the use of pre-mRNA-splicing factor SYF2 polypeptides or polypeptides that can interact with pre-mRNA-splicing factor SYF2 (e.g., to modulate its biological activity and/or its interaction with other biomolecules).

In some embodiments, examples of polypeptides (e.g., peptides; proteins, such as enzymes; antibodies; etc.) useful in the treatment of FTD include pre-mRNA-splicing factor SYF2 protein and functional fragments thereof.

Certain polypeptides provided in this section may be useful in treating FTD by modulating the activity of genes and gene products other than SYF2 genes/gene products, such as other genes/gene products associated with FTD.

### Small Molecules

SYF2 expression and/or activity in some embodiments can be modulated by the use of small molecules that can modulate pre-mRNA-splicing factor SYF2 protein (e.g., to modulate its biological activity, its expression, and/or its interaction with other biomolecules).

In some embodiments, examples of small molecules useful in the treatment of FTD are small molecules that increase or decrease expression of SYF2, and/or that increase or decrease pre-mRNA-splicing factor SYF2 protein levels or activity.

Certain small molecules provided in this section may be useful in treating FTD by modulating the activity of genes and gene products other than SYF2 genes/gene products, such as other genes/gene products associated with FTD.

### Gene Therapies

SYF2 expression and/or activity in some embodiments can be modulated by the use of gene therapies that can modulate SYF2 (e.g., by delivery of nucleic acids encoding SYF2 or other molecules that interact with SYF2).

In some embodiments, gene therapies, such as those involving administration of a compounds encoding useful therapeutic agents, useful in the treatment of FTD include payloads which encode pre-mRNA-splicing factor SYF2 protein or functional fragments thereof.

Certain gene therapies provided in this section may be useful in treating FTD by modulating the activity of genes and gene products other than SYF2 genes/gene products, such as other genes/gene products associated with FTD.

### Molecular Payloads Targeting UNC13A

The UNC13A gene, which encodes the unc-13 homolog A protein, and mutations therein, are implicated in FTD. Modulation of UCN13A expression and activity (e.g., by suppressing the expression and/or activity of mutant unc-13 homolog A protein and/or its interactions with other proteins) therefore in some embodiments can have a therapeutic effect in subjects with FTD.

### Oligonucleotides

UNC13A (and/or unc-13 homolog A protein encoded by UNC13A) expression and/or activity in some embodiments can be modulated by the use of oligonucleotides targeting UNC13A sequences.

In some embodiments, an oligonucleotide useful for the treatment of FTD, e.g., targeting (e.g., directly or indirectly modulating the expression or activity of) UNC13A, comprises a region of complementarity to an UNC13A transcript provided in Table 3, e.g., provided by any one of SEQ ID NOs: 169 and 810-818.

In some embodiments, examples of oligonucleotides useful for the treatment of FTD, e.g., targeting (e.g., directly or indirectly modulating the expression or activity of) UNC13A, are provided in WO2022246251A2, published Nov. 24, 2022, entitled “Compounds for modulating unc13a expression”; WO2023102225, published Dec. 2, 2022, entitled “Treatment of neurological diseases using modulators of unc13a gene transcripts”; US20230125137, published Apr. 27, 2023, entitled “Unc13a antisense oligonucleotides”; WO2022122872, published Jun. 16, 2022, entitled “Therapeutics for the treatment of neurodegenerative disorders”; WO2023102242, published Jun. 8, 2023, entitled “Splice switcher antisense oligonucleotides with modified backbone chemistries”; WO2023104964, published Jun. 15, 2023, entitled “Therapeutics for the treatment of neurodegenerative disorders”; and US20220033818A1, published Feb. 3, 2023, entitled “Oligonucleotides targeting ma binding protein sites”; the entire contents of which are herein incorporated by reference.

Certain oligonucleotides provided in this section may be useful in treating FTD by modulating the activity of genes and/or gene products other than UNC13A genes/gene products, such as other genes/gene products associated with FTD.

### Polypeptides

UNC13A expression and/or activity in some embodiments can be modulated by the use of unc-13 homolog A polypeptides or polypeptides that can interact with unc-13 homolog A (e.g., to modulate its biological activity and/or its interaction with other biomolecules).

In some embodiments, examples of polypeptides (e.g., peptides; proteins, such as enzymes; antibodies; etc.) useful in the treatment of FTD include unc-13 homolog A protein and functional fragments thereof.

Certain polypeptides provided in this section may be useful in treating FTD by modulating the activity of genes and gene products other than UNC13A genes/gene products, such as other genes/gene products associated with FTD.

### Small Molecules

UNC13A expression and/or activity in some embodiments can be modulated by the use of small molecules that can modulate unc-13 homolog A protein (e.g., to modulate its biological activity, its expression, and/or its interaction with other biomolecules).

In some embodiments, examples of small molecules useful in the treatment of FTD are small molecules that increase or decrease expression of UNC13A, and/or that increase or decrease unc-13 homolog A protein levels or activity.

Certain small molecules provided in this section may be useful in treating FTD by modulating the activity of genes and gene products other than UNC13A genes/gene products, such as other genes/gene products associated with FTD.

### Gene Therapies

UNC13A expression and/or activity in some embodiments can be modulated by the use of gene therapies that can modulate UNC13A (e.g., by delivery of nucleic acids encoding UNC13A or other molecules that interact with UNC13A).

In some embodiments, gene therapies, such as those involving administration of a compounds encoding useful therapeutic agents, useful in the treatment of FTD include payloads which encode unc-13 homolog A protein or functional fragments thereof.

Certain gene therapies provided in this section may be useful in treating FTD by modulating the activity of genes and gene products other than UNC13A genes/gene products, such as other genes/gene products associated with FTD.

### Molecular Payloads for the Treatment of Motor Neuron Disease

Various molecular payloads may be useful in the treatment of other motor neuron disease, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of motor neuron disease may include, in some embodiments, molecular payloads which modulate (e.g., increase or decrease) expression or activity of APOE, such as APOE4.

Examples of oligonucleotides useful for the treatment of motor neuron disease, e.g., oligonucleotides targeting (e.g., directly or indirectly modulating the expression or activity of) genes associated with motor neuron disease (e.g., APOE), include those listed in Table 14 below. Each oligonucleotide provided in Table 14 may have any modification pattern disclosed herein.

TABLE 14
Oligonucleotides for the treatment of motor neuron disease

| Target | |
|---|---|
| gene | Oligonucleotide Structure (5′-3′) |
| APOE | siRNA with sense strand: GAC CAU GAA GGA GUU GAA Gtt (SEQ ID NO: |
| | 170) |
| APOE | siRNA with target sequence: AAG GTG GAG CAA GCG GTG GAG (SEQ ID NO: |
| | 171) |
| APOE | siRNA with target sequence: GGA GTT GAA GGC CTA CAA A (SEQ ID NO: |
| | 172) |
| APOE | siRNA with target sequence: AAG CAG TTT GCC TGC AGA GAT (SEQ ID NO: |
| | 173) |

In each sequence listed in Table 14, each T may be optionally and independently replaced with a U, and each U may be optionally and independently replaced with a T.

Examples of small molecules useful for the treatment of motor neuron disease include small molecules which modulate expression and/or activity of APOE, or specifically of APOE4.

Examples of polypeptides useful for the treatment of motor neuron disease include antibodies, proteins, peptides, and enzymes.

### Molecular Payloads Targeting APOE

The APOE gene, and mutations therein, are implicated in motor neuron disease, which can affect upper motor neurons, lower motor neurons, or both. Modulation of APOE expression and activity (e.g., by suppressing the expression of APOE or a mutant form thereof and/or activity of a wild-type or mutant form of a protein encoded by APOE) therefore in some embodiments can have a therapeutic effect in subjects with motor neuron disease. In particular, allele-specific suppression of APOE can have a therapeutic effect in subjects with motor neuron disease. In some embodiments, treatment of motor neuron disease comprises allele-specific suppression of APOE4.

### Oligonucleotides

APOE expression and/or activity in some embodiments can be modulated by the use of oligonucleotides targeting APOE sequences.

In some embodiments, an oligonucleotide useful for the treatment of motor neuron disease, e.g., targeting (e.g., directly or indirectly modulating the expression or activity of) APOE, comprises a region of complementarity to an APOE transcript provided in Table 3, e.g., provided by any one of SEQ ID NOs: 501-505.

In some embodiments, examples of oligonucleotides useful for the treatment of motor neuron disease, e.g., targeting (e.g., directly or indirectly modulating the expression or activity of) APOE, are provided in Barger S W, et al. “Relationships between expression of apolipoprotein E and beta-amyloid precursor protein are altered in proximity to Alzheimer beta-amyloid plaques: potential explanations from cell culture studies.” J Neuropathol Exp Neurol. (2008) 67(8):773-83; Casey C S, et al. “Apolipoprotein E Inhibits Cerebrovascular Pericyte Mobility through a RhoA Protein-mediated Pathway.” J Biol Chem. (2015) 290(22):14208-17; International Patent Application Publication No. WO2022066956A1, published Mar. 31, 2022, entitled “Compounds and methods for reducing apoe expression”; International Patent Application Publication No. WO2021178374A2, published Sep. 10, 2021, entitled “Compounds and methods for reducing apoe expression”; U.S Patent Application Publication No. US20210155925A1, published May 27, 2021, entitled “Compositions and methods for disrupting the molecular mechanisms associated with mitochondrial dysfunction and neurodegenerative disease”; and U.S. Patent Application Publication No. US20170334977A1, published Nov. 23, 2017, entitled “Targeting Apolipoprotein E (APOE) in Neurologic Disease”; the entire contents of each of which are herein incorporated by reference.

Certain oligonucleotides provided in this section may be useful in treating motor neuron disease by modulating the activity of genes and/or gene products other than APOE genes/gene products, such as other genes/gene products associated with motor neuron disease.

### Polypeptides

APOE expression and/or activity in some embodiments can be modulated by the use of APOE polypeptides or polypeptides that can interact with APOE (e.g., to modulate its activity).

In some embodiments, examples of polypeptides (e.g., peptides; proteins, such as enzymes; antibodies; etc.) useful in the treatment of motor neuron disease are provided in Sadowski M J, at al. “Blocking the apolipoprotein E/amyloid-beta interaction as a potential therapeutic approach for Alzheimer's disease.” Proc Natl Acad Sci USA. (2006) 103(49):18787-92; U.S Patent Application Publication No. US20040214774A1, published Oct. 28, 2004, entitled “Prevention and treatment of Alzheimer amyloid deposition”; International Patent Application Publication No. WO2020243346A1, published Dec. 3, 2020, entitled “Apoe antibodies, fusion proteins and uses thereof”; U.S. Patent Application Publication No. US20190270794A1, published Sep. 21, 2021, entitled “Anti-apoe antibodies”; U.S. Pat. No. 10,040,836B2, published Aug. 7, 2018, entitled “Peptides for the treatment of neurodegenerative diseases”; U.S. Patent Application Publication No. US2014037638A1, published Feb. 6, 2014, entitled “Compositions and methods for treating amyloid plaque associated symptoms”; and U.S. Patent Application Publication No. US20100266505A1, published Oct. 21, 2010, entitled “Immunotherapy regimes dependent on apoe status”; the entire contents of each of which are herein incorporated by reference.

Certain polypeptides provided in this section may be useful in treating motor neuron disease by modulating the activity of genes and gene products other than APOE genes/gene products, such as other genes/gene products associated with motor neuron disease.

### Small Molecules

APOE expression and/or activity in some embodiments can be modulated by the use of small molecules that can modulate APOE (e.g., to modulate its activity, or its expression).

In some embodiments, examples of small molecules useful in the treatment of motor neuron disease are provided in U.S. Patent Application Publication No. US20210353566A1, published Nov. 18, 2021, entitled “The use of choline supplementation as therapy for apoe4-related disorders”; U.S. Patent Application Publication No. US20210085692A1, published Mar. 25, 2021, entitled “Agents, compositions and methods for treating and preventing Alzheimer's disease”; and U.S. Patent Application Publication No. US20180036274A1, published Oct. 30, 2018, entitled “Use of medium chain triglycerides for the treatment and prevention of Alzheimer's disease and other diseases resulting from reduced neuronal metabolism ii”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating motor neuron disease by modulating the activity of genes and gene products other than APOE genes/gene products, such as other genes/gene products associated with motor neuron disease.

### Gene Therapies

APOE expression and/or activity in some embodiments can be modulated by the use of gene therapies that can modulate APOE (e.g., by delivery of nucleic acids encoding APOE or other molecules that interact with APOE).

In some embodiments, gene therapies, such as those involving administration of compounds encoding useful therapeutic agents, useful in the treatment of motor neuron disease are provided in International Patent Application Publication No. WO2021067611A2, published Apr. 8, 2021, entitled “Compositions and methods for treating Alzheimer's disease”; and U.S. Patent Application Publication No. US2021195879A1, published Jul. 1, 2021, entitled “Genetically modified mouse models of Alzheimer's disease”; the entire contents of each of which are herein incorporated by reference.

Certain gene therapies provided in this section may be useful in treating motor neuron disease by modulating the activity of genes and gene products other than APOE genes/gene products, such as other genes/gene products associated with motor neuron disease.

### Molecular Payloads for the Treatment of Hereditary Dystonia

Various molecular payloads may be useful in the treatment of hereditary dystonia, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of essential tremor may include, in some embodiments, molecular payloads which modulate (e.g., increase or decrease) expression or activity of TOR1A, THAP1, ANO3, GNAL, KMT2B, GCH1, TH, SPR, TAF1, PRKRA, ATP1A3, SGCE, PNKD, PRRT2, SLC2A1, and/or ECHS1.

Examples of molecular payloads useful for the treatment of hereditary dystonia, e.g., molecular payloads targeting (e.g., directly or indirectly modulating the expression or activity of) genes associated with hereditary dystonia (e.g., TOR1A, THAP1, ANO3, GNAL, KMT2B, GCH1, TH, SPR, TAF1, PRKRA, ATP1A3, SGCE, PNKD, PRRT2, SLC2A1, ECHS1, etc.), include those provided in US2019/0160184A1, entitled “Means and Methods to Treat Dystonia”, published May 30, 2019; US2005/0106731A1, entitled “siRNA-Mediated Gene Silencing with Viral Vectors”, published May 19, 2005; US2016/0032319A1, published Feb. 4, 2016, entitled “Vectors comprising stuffer/filler polynucleotide sequences and methods of use”; US2019/0241633A1, published Aug. 8, 2019, entitled “RNA Encoding a Therapeutic Protein”; US2016/0032319A1, published Feb. 4, 2016, entitled “Vectors comprising stuffer/filler polynucleotide sequences and methods of use”; US 2005/0208584A1, published Sep. 22, 2005, entitled “Chemokine-binding proteins and methods of use”; US2003/0186337A1, published Oct. 2, 2003, entitled “Novel death associated proteins, and THAP1 and PAR4 pathways in apoptosis control”; US20060270595A1, published Nov. 30, 2006, entitled “Nucleic acids encoding compositions of THAP-family chemokine binding domains”; US20160024496A1, published Jan. 28, 2016, entitled “Methods for monitoring c9orf72 expression”; US20190240293A1, published Aug. 8, 2019, entitled “Neuromodulating compositions and related therapeutic methods for the treatment of cancer by modulating an anti-cancer immune response”; US20190032079A1, published Jan. 31, 2019, entitled “Systemic synthesis and regulation of 1-dopa”; US20150111878A1, published Apr. 23, 2015, entitled “Compositions and methods for treating intestinal hyperpermeability”; US20170306301A1, published Oct. 26, 2017, entitled “Tyrosine hydroxylase variants and methods of use thereof”; US20130184214A1, published Jul. 18, 2013, entitled “Pharmaceutical compositions and methods”; US20170307591A1, published Oct. 26, 2017, entitled “Methods and assays relating to sepiapterin reductase inhibition”; US20120322800A1, published Dec. 20, 2012, entitled “Sepiapterin reductase inhibitors for the treatment of pain”; US20170096435A1, published Apr. 6, 2017, entitled “Sepiapterin reductase inhibitors”; US20050197341A1, published Sep. 8, 2005, entitled “Methods for treating pain”; US20040014167A1, published Jan. 22, 2004, entitled “Process for producing biopterin”; U.S. Ser. No. 10/702,518B2, published Jul. 7, 2020, entitled “TAF1 inhibitors for the therapy of cancer”; WO2021189036A1, published Sep. 23, 2021, entitled “Taf1 inhibitors”; US20190192502A1, published Jun. 27, 2019, entitled “Modulation of Transcription Initiation Factor TFIID Subunit 1 (TAF1) for Treating Leukemia”; WO2022115753A1, published Jun. 2, 2022, entitled “Merged scaffold taf1 inhibitors”; U.S. Ser. No. 10/202,378B2, published Feb. 12, 2019, entitled “Therapeutic compounds and uses thereof”; US20070179091A1, published Aug. 2, 2007, entitled “Hedgehog Kinases and Their Use in Modulating Hedgehog Signaling”; US20210155932A1, published May 7, 2021, entitled “Compositions and methods to treat cancer”; US20190358346A1, published Nov. 28, 2019, entitled “Aav-mediated delivery of atp1a3 genes to central nervous system”; US20220088152A1, published Mar. 24, 2022, entitled “Compositions and methods for the treatment of atpase-mediated diseases”; WO2017106382A1, published Jun. 22, 2017, entitled “Compositions and methods for treatment of central nervous system diseases”; US20190070213A1, published Mar. 7, 2019, entitled “Antisense oligomers and uses thereof”; US20190160078A1, published May 30, 2019, entitled “Ganaxolone for use in treating genetic epileptic disorders”; US20210222167A1, published Jul. 22, 2021, entitled “Slc2a1 lncrna as a biologic and related treatments and methods”; US20180042991A1, published Feb. 15, 2018, entitled “Recombinant glut1 adeno-associated viral vector constructs and related methods for restoring glut1 expression”; WO2016152293A1, published Sep. 29, 2016, entitled “Composition and method for inhibiting glut1 expression by cancer cells”; WO2022031760A1, published Feb. 10, 2022, entitled “Adeno-associated viral vector for glut1 expression and uses thereof”; WO2021136763A1, published Jul. 8, 2021, entitled “Isoquinoline derivatives for use in treating glut1 deficiency syndrome”; WO2011060599A1, published May 26, 2011, entitled “Small interfering rna(sirna) which can specifically inhibit expression of echs1 gene and use thereof”; the entire contents of each of which are herein incorporated by reference.

Certain molecular payloads provided in this section may be useful in treating hereditary dystonia by modulating the activity of genes and/or gene products other than TOR1A, THAP1, ANO3, GNAL, KMT2B, GCH1, TH, SPR, TAF1, PRKRA, ATP1A3, SGCE, PNKD, PRRT2, SLC2A1, and/or ECHS1 genes/gene products. For example, a molecular payload may be useful in treating hereditary dystonia by modulating the activity of genes and/or gene products that interact with (e.g., form biological complexes with, promote expression of, suppress expression of, etc.) TOR1A, THAP1, ANO3, GNAL, KMT2B, GCH1, TH, SPR, TAF1, PRKRA, ATP1A3, SGCE, PNKD, PRRT2, SLC2A1, and/or ECHS1 genes/gene products.

### Molecular Payloads for the Treatment of Epilepsy and Pain Disorders

Various molecular payloads may be useful in the treatment of epilepsy and/or pain disorders, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of epilepsy may include, in some embodiments, molecular payloads which modulate (e.g., increase or decrease) expression or activity of SCN1A, SCN2A, SCN8A, SCN9A, CLN3, GRIA1, and/or PCDH19. Many molecular payloads useful in the treatment of epilepsy may also be useful in the treatment of pain disorders. Such molecular payloads include molecular payloads that can modulate expression or activity of SCN1A, SCN2A, SCN8A, SCN9A, CLN3, GRIA1, and/or PCDH19. Molecular payloads useful in the treatment of epilepsy and/or in the treatment of pain disorders may also include, in some embodiments, molecular payloads which modulate (e.g., increase or decrease) expression or activity of GRIN2A.

Examples of oligonucleotides useful for the treatment of epilepsy and/or pain disorders, e.g., oligonucleotides targeting (e.g., directly or indirectly modulating the expression or activity of) genes associated with epilepsy and/or pain disorders (e.g., SCN1A, SCN2A, SCN8A, SCN9A, CLN3, GRIA1, PCDH19, etc.), include those listed in Table 15 below. Each oligonucleotide provided in Table 15 may have any modification pattern disclosed herein.

TABLE 15
Oligonucleotides for the treatment of epilepsy and/or pain disorders

| | | Oligo- | |
|---|---|---|---|
| SEQ | | nucleotide | |
| ID | Target | Sequence | |
| NO: | gene | (5′-3′) | Oligonucleotide Structure (5′-3′) |
| 181 | SCN1A | AGTTGGAGCA | Ans Gns Tns Tns Gns Gns Ans Gns mCns Ans Ans Gns |
| | | AGATTATC | Ans Tns Tns Ans Tns mCn |
| 182 | SCN1A | AGTTGGAGCA | Aes Geo Teo Tes Ges Ges Aes Ges mCes Aes Aes Ges |
| | | AGATTATC | Aes Tes Tes Aes Tes mCe |
| 183 | SCN1A | AGTTGGAGCA | Aes Geo Tes Teo Ges Ges Aes Ges mCes Aes Aes Ges |
| | | AGATTATC | Aes Tes Tes Aes Tes mCe |
| 184 | SCN1A | AGTTGGAGCA | Aes Geo Tes Aes Tes mCe Aes Ges mCes Aes Aes Ges |
| | | AGATTATC | Aes Tes Tes Tes Ges Geo |
| 185 | SCN1A | AGTTGGAGCA | Aes Geo Tes Tes Ges Ges Aes Geo mCes Aes Aes Ges |
| | | AGATTATC | Aes Tes Tes Aes Tes mCe |
| 186 | SCN1A | AGTTGGAGCA | Aes Ges Tes Aes Tes mCe Aes Ges mCes Aes Aes Ges |
| | | AGATTATC | Aes Tes Tes Teo Geo Ges |
| 187 | SCN1A | AGTTGGAGCA | Aes Ges Tes Tes Ges Ges Aes Geo mCeo Aes Aes Ges |
| | | AGATTATC | Aes Tes Tes Aes Tes mCe |
| 188 | SCN1A | AGTTGGAGCA | Aes Ges Tes Tes Ges Ges Aes Ges mCes Aeo Aeo Ges |
| | | AGATTATC | Aes Tes Tes Aes Tes mCe |
| 189 | SCN1A | AGTTGGAGCA | Aes Ges Tes Tes Ges mCe Aes Ges mCes Aes Aes Geo |
| | | AGATTATC | Aeo Tes Tes Aes Tes Ges |
| 190 | SCN1A | AGTTGGAGCA | Aes Geo Tes Tes Ges Ges Aes Ges mCes Aeo Aes Ges |
| | | AGATTATC | Aes Tes Tes Aes Tes mCe |
| 191 | SCN1A | AGTTGGAGCA | Aes Geo Tes Tes Ges Ges Aes Ges mCes Aes Aes Geo |
| | | AGATTATC | Aes Tes Tes Aes Tes mCe |
| 192 | SCN1A | AGTTGGAGCA | Aes Geo Tes Tes Ges Ges Aes Ges mCes Aes Aes Ges |
| | | AGATTATC | Aes Tes Teo Aes Tes mCe |
| 193 | SCN1A | AGTTGGAGCA | Aes Ges Teo Tes Ges Ges Aes Ges mCes Aes Aes Ges |
| | | AGATTATC | Aes Tes Teo Aes Tes mCe |
| 194 | SCN1A | AGTTGGAGCA | Aes Ges Tes Tes Geo Ges Aes Ges mCes Aes Aes Ges |
| | | AGATTATC | Aes Tes Teo Aes Tes mCe |
| 195 | SCN1A | AGTTGGAGCA | Aes Ges Tes Tes Ges Geo Aeo Ges mCes Aes Aes Ges |
| | | AGATTATC | Aes Tes Tes Aes Tes mCe |
| 196 | SCN1A | AGTTGGAGCA | Aes Ges Tes Tes Ges Ges Aeo Ges mCes Aes Aes Ges |
| | | AGATTATC | Aes Tes Teo Aes Tes mCe |
| 197 | SCN1A | AGTTGGAGCA | Aes Ges Tes Tes Ges Ges Aes Ges mCeo Aes Aes Ges |
| | | AGATTATC | Aes Teo Tes Aes Tes mCe |
| 198 | SCN1A | AGTTGGAGCA | Aes Ges Tes Aes Tes mCe Aes Ges mCeo Aes Aes Ges |
| | | AGATTATC | Aes Tes Teo Tes Ges Ges |
| 199 | SCN1A | AGTTGGAGCA | Aes Ges Tes Aes Tes mCe Aes Ges mCes Aes Aeo Ges |
| | | AGATTATC | Aes Tes Teo Tes Ges Ges |
| 200 | SCN1A | AGTTGGAGCA | Aes Ges Tes Tes Ges Ges Aes Ges mCes Aes Aes Geo |
| | | AGATTATC | Aes Teo Tes Aes Tes mCe |
| 201 | SCN1A | AGTTGGAGCA | Aes Ges Tes Tes Ges Ges Aes Ges mCes Aes Aes Ges |
| | | AGATTATC | Aeo Tes Teo Aes Tes mCe |
| 202 | SCN1A | AGTTGGAGCA | Aes Ges Tes Tes Ges Ges Aes Ges mCes Aes Aes Ges |
| | | AGATTATC | Aes Teo Teo Aes Tes mCe |
| 203 | SCN1A | AAGTTGGAGC | Aeo Aes Ges Tes Tes Ges Ges Aes Ges mCes Aes Aes |
| | | AAGATTATCC | Ges Aes Tes Tes Aes Tes mCeo mCe |
| 204 | SCN1A | AGTTGGAGCA | Aes Ges Tes Tes Ges Ges Aes Ges mCes Aes Aes Ges |
| | | AGATTATCC | Aes Tes Tes Aes Tes mCeo mCe |
| 205 | SCN1A | AAGTTGGAGC | Aeo Aes Ges Tes Tes Ges Ges Aes Ges mCes Aes Aes |
| | | AAGATTATC | Ges Aes Tes Tes Aes Tes mCe |
| 206 | SCN1A | GGTAGCAAAA | Gns Gns Tns Ans Gns mCns Ans Ans Ans Ans Gns Gns |
| | | GGGGTAAT | Gns Gns Tns Ans Ans Tn |
| 207 | SCN1A | AGCAAAAGGG | Ans Gns mCns Ans Ans Ans Ans Gns Gns Gns Gns Tns |
| | | GTAATACA | Ans Ans Tns Ans mCns An |
| 208 | SCN1A | GCAAAAGGGG | Gns mCns Ans Ans Ans Ans Gns Gns Gns Gns Tns Ans |
| | | TAATACAG | Ans Tns Ans mCns Ans Gn |
| 209 | SCN1A | CAAAAGGGGT | mCns Ans Ans Ans Ans Gns Gns Gns Gns Tns Ans Ans |
| | | AATACAGT | Tns Ans mCns Ans Gns Tn |
| 210 | SCN1A | AAAGGGGTAA | Ans Ans Ans Gns Gns Gns Gns Tns Ans Ans Tns Ans |
| | | TACAGTAC | mCns Ans Gns Tns Ans mCn |
| 211 | SCN1A | AAGGGGTAAT | Ans Ans Gns Gns Gns Gns Tns Ans Ans Tns Ans mCns |
| | | ACAGTACC | Ans Gns Tns Ans mCns mCn |
| 212 | SCN1A | GTAATACAGT | Gns Tns Ans Ans Tns Ans mCns Ans Gns Tns Ans mCns |
| | | ACCCATAA | mCns mCns Ans Tns Ans An |
| 213 | SCN1A | TAATACAGTA | Tns Ans Ans Tns Ans mCns Ans Gns Tns Ans mCns |
| | | CCCATAAT | mCns mCns Ans Tns Ans Ans Tn |
| 214 | SCN1A | AATACAGTAC | Ans Ans Tns Ans mCns Ans Gns Tns Ans mCns mCns |
| | | CCATAATA | mCns Ans Tns Ans Ans Tns An |
| 215 | SCN1A | ATACAGTACC | Ans Tns Ans mCns Ans Gns Tns Ans mCns mCns mCns |
| | | CATAATAA | Ans Tns Ans Ans Tns Ans An |
| 216 | SCN1A | TACAGTACCC | Tns Ans mCns Ans Gns Tns Ans mCns mCns mCns Ans |
| | | ATAATAAA | Tns Ans Ans Tns Ans Ans An |
| 217 | SCN1A | ACAGTACCCA | Ans mCns Ans Gns Tns Ans mCns mCns mCns Ans Tns |
| | | TAATAAAG | Ans Ans Tns Ans Ans Ans Gn |
| 218 | SCN1A | CCCATCCAAG | mCns mCns mCns Ans Tns mCns mCns Ans Ans Gns Tns |
| | | TTGGAGCA | Tns Gns Gns Ans Gns mCns An |
| 219 | SCN1A | CCATCCAAGT | mCns mCns Ans Tns mCns mCns Ans Ans Gns Tns Tns |
| | | TGGAGCAA | Gns Gns Ans Gns mCns Ans An |
| 220 | SCN1A | CATCCAAGTT | mCns Ans Tns mCns mCns Ans Ans Gns Tns Tns Gns |
| | | GGAGCAAG | Gns Ans Gns mCns Ans Ans Gn |
| 221 | SCN1A | ATCCAAGTTG | Ans Tns mCns mCns Ans Ans Gns Tns Tns Gns Gns Ans |
| | | GAGCAAGA | Gns mCns Ans Ans Gns An |
| 222 | SCN1A | TCCAAGTTGG | Tns mCns mCns Ans Ans Gns Tns Tns Gns Gns Ans Gns |
| | | AGCAAGAT | mCns Ans Ans Gns Ans Tn |
| 223 | SCN1A | CCAAGTTGGA | mCns mCns Ans Ans Gns Tns Tns Gns Gns Ans Gns |
| | | GCAAGATT | mCns Ans Ans Gns Ans Tns Tn |
| 224 | SCN1A | CAAGTTGGAG | mCns Ans Ans Gns Tns Tns Gns Gns Ans Gns mCns Ans |
| | | CAAGATTA | Ans Gns Ans Tns Tns An |
| 225 | SCN1A | AAGTTGGAGC | Ans Ans Gns Tns Tns Gns Gns Ans Gns mCns Ans Ans |
| | | AAGATTAT | Gns Ans Tns Tns Ans Tn |
| 226 | SCN1A | TTGGAGCAAG | Tns Tns Gns Gns Ans Gns mCns Ans Ans Gns Ans Tns |
| | | ATTATCCT | Tns Ans Tns mCns mCns Tn |
| 227 | SCN1A | TGGAGCAAGA | Tns Gns Gns Ans Gns mCns Ans Ans Gns Ans Tns Tns |
| | | TTATCCTA | Ans Tns mCns mCns Tns An |
| 228 | SCN1A | GGAGCAAGAT | Gns Gns Ans Gns mCns Ans Ans Gns Ans Tns Tns Ans |
| | | TATCCTAT | Tns mCns mCns Tns Ans Tn |
| 229 | SCN1A | GAGCAAGATT | Gns Ans Gns mCns Ans Ans Gns Ans Tns Tns Ans Tns |
| | | ATCCTATA | mCns mCns Tns Ans Tns An |
| 230 | SCN2A | GCATAATCCC | Ges mCeo Aeo Teo Aeo Ads Tds mCds mCds mCds Ads |
| | | ATTATACAAA | Tds Tds Ads Tds Aeo mCeo Aes Aes Ae |
| 231 | SCN2A | CACGACATAT | mCes Aeo mCeo Geo Aeo mCeo Ads Tds Ads Tds Tds |
| | | TTTTCTACAC | Tds Tds Tds mCds Tds Aeo mCes Aes mCe |
| 232 | SCN2A | CCACGACATA | mCes mCeo Aeo mCeo Geo Aeo mCds Ads Tds Ads Tds |
| | | TTTTTCTACA | Tds Tds Tds Tds mCds Teo Aes mCes Ae |
| 233 | SCN2A | TCTGCATGTA | Tes mCeo Teo Geo mCeo Aeo Tds Gds Tds Ads Ads |
| | | ACCTTTATAC | mCds mCds Tds Tds Tds Aeo Tes Aes mCe |
| 234 | SCN2A | GCATAATCCC | Ges mCeo Aeo Teo Aeo Aeo Tds mCds mCds mCds Ads |
| | | ATTATACAAA | Tds Tds Ads Tds Ads mCeo Aes Aes Ae |
| 235 | SCN2A | CTGCATGTAA | mCes Teo Geo mCeo Aes Tds Gds Tds Ads Ads mCds |
| | | CCTTTATA | mCds Tds Teo Teo Aes Tes Ae |
| 236 | SCN8A | GACGATTAGT | Ges Aes Ces Ges Aes Tds Tds Ads Gds Tds Gds Ads |
| | | GACATAGGCT | Cds Ads Tds Aes Ges Ges Ces Te |
| 237 | SCN8A | GACGATTAGT | Gex Aex Cex Gex Aex Tdx Tdx Adx Gdx Tdx Gdx Adx |
| | | GACATAGGCT | Cdx Adx Tdx Aex Gex Gex Cex Te |
| | SCN8A | | Antisense oligonucleotide complementary to the target |
| | | | sequence |
| | | | UGAUAAGGCUCUUAAGGGUCUCAAAGCUCUUAGGGUCCUAAGGGACUUU |
| | | | AUGGCACCUAGUUCCGAGUAGCCCAGGGCA (SEQ ID NO: 238) |
| | | | and comprising a nucleobase sequence UCCGAGUAGCCCAGG |
| | | | (SEQ ID NO: 288) |
| 239 | SCN8A | GCACCUAGUU | LNA, 2′-OMe, or PMO antisense oligonucleotide comprising |
| | | CCGAGUAGCC | a nucleobase sequence GCACCUAGUUCCGAGUAGCCCAGG (SEQ |
| | | CAGG | ID NO: 289) |
| 240 | SCN8A | CACCUAGUUC | LNA, 2′-OMe, or PMO antisense oligonucleotide comprising |
| | | CGAGUAGCCC | a nucleobase sequence CACCUAGUUCCGAGUAGCCCAGGG (SEQ |
| | | AGGG | ID NO: 290) |
| 241 | SCN8A | ACCUAGUUCC | LNA, 2′-OMe, or PMO antisense oligonucleotide comprising |
| | | GAGUAGCCCA | a nucleobase sequence ACCUAGUUCCGAGUAGCCCAGGG (SEQ ID |
| | | GGG | NO: 291) |
| 242 | SCN8A | CCUAGUUCCG | LNA, 2′-OMe, or PMO antisense oligonucleotide comprising |
| | | AGUAGCCCAG | a nucleobase sequence CCUAGUUCCGAGUAGCCCAGGG (SEQ ID |
| | | GG | NO: 292) |
| 243 | SCN8A | CUAGUUCCGA | LNA, 2′-OMe, or PMO antisense oligonucleotide comprising |
| | | GUAGCCCAGG | a nucleobase sequence CUAGUUCCGAGUAGCCCAGGGC (SEQ ID |
| | | GC | NO: 293) |
| 244 | SCN8A | UAGUUCCGAG | LNA, 2′-OMe, or PMO antisense oligonucleotide comprising |
| | | UAGCCCAGGG | a nucleobase sequence UAGUUCCGAGUAGCCCAGGGCA (SEQ ID |
| | | CA | NO: 294) |
| 245 | SCN8A | CUAGUUCCGA | LNA, 2′-OMe, or PMO antisense oligonucleotide comprising |
| | | GUAGCCCAGG | a nucleobase sequence CUAGUUCCGAGUAGCCCAGGG (SEQ ID |
| | | G | NO: 295) |
| 246 | SCN8A | UAGUUCCGAG | LNA, 2′-OMe, or PMO antisense oligonucleotide comprising |
| | | UAGCCCAGGG | a nucleobase sequence UAGUUCCGAGUAGCCCAGGG (SEQ ID |
| | | | NO: 296) |
| 247 | SCN8A | UCCGAGUAGC | LNA, 2′-OMe, or PMO antisense oligonucleotide comprising |
| | | CCAGG | a nucleobase sequence UCCGAGUAGCCCAGG (SEQ ID NO: |
| | | | 297) |
| 248 | CLN3 | ACAACCCTCC | Aes Ces Aes Aes Ces Ces Ces Tes Ces Ces Ces Aes |
| | | CAACCACG | Aes Ces Ces Aes Ces Ge |
| 249 | CLN3 | GGGACAACCC | Ges Ges Ges Aes Ces Aes Aes Ces Ces Ces Tes Ces |
| | | TCCCAACC | Ces Ces Aes Aes Ces Ce |
| 250 | CLN3 | AGGGGACAAC | Aes Ges Ges Ges Ges Aes Ces Aes Aes Ces Ces Ces |
| | | CCTCCCAA | Tes Ces Ces Ces Aes Ae |
| 251 | CLN3 | CTTCCAGGGG | Ces Tes Tes Ces Ces Aes Ges Ges Ges Ges Aes Ces |
| | | ACAACCCT | Aes Aes Ces Ces Ces Te |
| 252 | CLN3 | CAGAGCTTCC | Ces Aes Ges Aes Ges Ces Tes Tes Ces Ces Aes Ges |
| | | AGGGGACA | Ges Ges Ges Aes Ces Ae |
| 253 | CLN3 | GACCGCAGAG | Ges Aes Ces Ces Ges Ces Aes Ges Aes Ges Ces Tes |
| | | CTTCCAGG | Tes Ces Ces Aes Ges Ge |
| 254 | CLN3 | AGTGAGACCG | Aes Ges Tes Ges Aes Ges Aes Ces Ces Ges Ces Aes |
| | | CAGAGCTT | Ges Aes Ges Ces Tes Te |
| 255 | CLN3 | AATAGAGTGA | Aes Aes Tes Aes Ges Aes Ges Tes Ges Aes Ges Aes |
| | | GACCGCAG | Ces Ces Ges Ces Aes Ge |
| 256 | CLN3 | AGGAGAATAG | Aes Ges Ges Aes Ges Aes Aes Tes Aes Ges Aes Ges |
| | | AGTGAGAC | Tes Ges Aes Ges Aes Ce |
| 257 | CLN3 | GGGACAGGAG | Ges Ges Ges Aes Ces Aes Ges Ges Aes Ges Aes Aes |
| | | AATAGAGT | Tes Aes Ges Aes Ges Te |
| 258 | CLN3 | AGCCTGGGAC | Ges Ges Ces Ces Tes Ges Ges Ges Aes Ces Aes Ges |
| | | AGGAGAAT | Aes Aes Ges Aes Aes Te |
| 259 | CLN3 | AGCACAGCCT | Aes Ges Ces Aes Ces Aes Ges Ces Ces Tes Ges Ges |
| | | GGGACAGG | Ges Aes Ces Aes Ges G? |
| 260 | CLN3 | CCAGGAGCAC | Ces Ces Aes Ges Ges Aes Ges Ces Aes Ces Aes Ges |
| | | AGCCTGGG | Ces Ces Tes Ges Ges G? |
| 261 | CLN3 | GTCCGCCAGG | Ges Tes Ces Ces Ges Ces Ces Aes Ges Ges Aes Ges |
| | | AGCACAGC | Ces Aes Ces Aes Ges Ce |
| 262 | CLN3 | AGGATGTCCG | Aes Ges Ges Aes Tes Ges Tes Ces Ces Ges Ces Ces |
| | | CCAGGAGC | Aes Ges Ges Aes Ges Ces |
| 263 | CLN3 | GGGAGGATGT | Ges Ges Ges Aes Ges Ges Aes Tes Ges Tes Ces Ces |
| | | CCGCCAGG | Ges Ces Ces Aes Ges G? |
| 264 | CLN3 | TGGGGAGGAT | Tes Ges Ges Ges Ges Aes Ges Ges Aes Tes Ges Tes |
| | | GTCCGCCA | Ces Ces Ges Ces Ces Ae |
| 265 | CLN3 | GAGTGTGGGG | Ges Aes Ges Tes Ges Tes Ges Ges Ges Ges Aes Ges |
| | | AGGATGTC | Ges Aes Tes Ges Tes Ce |
| 266 | CLN3 | ATGACGAGTG | Aes Tes Ges Aes Ces Ges Aes Ges Tes Ges Tes Ges |
| | | TGGGGAGG | Ges Ges Ges Aes Ges G? |
| 267 | CLN3 | ATTTGATGAC | Aes Tes Tes Tes Ges Aes Tes Ges Aes Ces Ges Aes |
| | | GAGTGTGG | Ges Tes Ges Tes Ges Ge |
| 268 | CLN3 | CAACAATTTG | Ces Aes Aes Ces Aes Aes Tes Tes Tes Ges Aes Tes |
| | | ATGACGAG | Ges Aes Ces Ges Aes Ge |
| 269 | CLN3 | GGAGCCAACA | Ges Ges Aes Ges Ces Ces Aes Aes Ces Aes Aes Tes |
| | | ATTTGATG | Tes Tes Ges Aes Tes Ge |
| 270 | CLN3 | CAAGAGGAGC | Aes Aes Aes Ges Aes Ges Ges Aes Ges Ces Ces Aes |
| | | CAACAATT | Ces Ces Aes Aes Tes Te |
| 271 | CLN3 | AAGGCCAAGA | Aes Aes Ges Ges Ces Ces Aes Aes Ges Aes Ges Ges |
| | | GGAGCCAA | Aes Ges Ces Ces Aes Ae |
| 272 | CLN3 | AGGTGAAGGC | Aes Ges Ges Tes Ges Aes Aes Ges Ges Ces Ces Aes |
| | | CAAGAGGA | Aes Ges Aes Ges Ges Ae |
| 273 | CLN3 | GCAGCAGGTG | Ges Ces Aes Ges Ces Aes Ges Ges Tes Ges Aes Aes |
| | | AAGGCCAA | Ges Ges Ces Ces Aes Ae |
| 274 | CLN3 | GTAGGGCAGC | Ges Tes Aes Ges Ges Ges Ces Aes Ges Ces Aes Ges |
| | | AGGTGAAG | Ges Tes Ges Aes Aes G? |
| 275 | CLN3 | GACCTGTAGG | Ges Aes Ces Ces Tes Ges Tes Aes Ges Ges Ges Ces |
| | | GCAGCAGG | Aes Ges Ces Aes Ges Ge |
| 276 | CLN3 | ACCCAGACCT | Aes Ces Ces Ces Aes Ges Aes Ces Ces Tes Ges Tes |
| | | GTAGGGCA | Aes Ges Ges Ges Ces Ae |
| 277 | CLN3 | CCCTCACCCA | Ces Ces Ces Tes Ces Aes Ces Ces Ces Aes Ges Aes |
| | | GACCTGTA | Ces Ces Tes Ges Tes Ae |
| 278 | CLN3 | CACTACCCTC | Ces Aes Ces Tes Aes Ces Ces Ces Tes Ces Aes Ces |
| | | ACCCAGAC | Ces Ces Aes Ges Aes Ce |
| 279 | CLN3 | CCTCCCACTA | Ces Ces Tes Ces Ces Ces Aes Ces Tes Aes Ces Ces |
| | | CCCTCACC | Ces Tes Ces Aes Ces Ce |
| 280 | CLN3 | CCCTGCCTCC | Ces Ces Ces Tes Ges Ces Ces Tes Ces Ces Ces Aes |
| | | CACTACCC | Ces Tes Aes Ces Ces Ce |
| 281 | CLN3 | GCCCACCCTG | Ges Ces Ces Ces Aes Ces Ces Ces Tes Ges Ces Ces |
| | | CCTCCCAC | Tes Ces Ces Ces Aes Ce |
| 282 | CLN3 | CTCCTGCCCA | Ces Tes Ces Ces Tes Ges Ces Ces Ces Aes Ces Ces |
| | | CCCTGCCT | Ces Tes Ges Ces Ces Te |
| 283 | CLN3 | CTCAGCTCCT | Ces Tes Ces Aes Ges Ces Tes Ces Ces Tes Ges Ces |
| | | GCCCACCC | Ces Ces Aes Ces Ces Ce |
| 284 | CLN3 | CCTTTCTCAG | Ces Ces Tes Tes Tes Ces Tes Ces Aes Ges Ces Tes |
| | | CTCCTGCC | Ces Ces Tes Ges Ces Ce |
| 285 | CLN3 | CCTCCCCTTT | Ces Aes Ges Ces Tes Ces Ces Tes Tes Tes Ces Tes |
| | | CTCAGCTC | Ces Ces Tes Ces Ces Ce |
| 286 | CLN3 | CCCAGCCTCC | Ces Ces Ces Aes Ges Ces Ces Tes Ces Ces Ces Ces |
| | | CCTTTCTC | Tes Tes Tes Ces Tes Ce |
| 287 | CLN3 | GCCATCCCAG | Ges Ces Ces Aes Tes Ces Ces Ces Aes Ges Ces Ces |
| | | CCTCCCCT | Tes Ces Ces Ces Ces Te |
| | GRIA1 | | siRNA comprising: |
| | | | sense sequence: GUACACAUCUGCUCUGACCUACGAT (SEQ ID |
| | | | NO: 298) |
| | | | and antisense sequence: AUCGUAGGUCAGAGCAGAUGUGUACU |
| | | | (SEQ ID NO: 299) |

Abbreviations used in Table 15:
mC = 5-methyl cytosine; n = 2′-NMA sugar moiety; s = phosphorothioate internucleoside linkage; o = phosphodiester internucleoside linkage; s = phosphorothioate internucleoside linkage; x = internucleoside linkage that is either phosphorothioate or phosphodiester; e = 2′-MOE modified sugar. In each sequence listed in Table 15, each T may be optionally and independently replaced with a U, and each U may be optionally and independently replaced with a T.

Examples of small molecules useful for the treatment of epilepsy and/or pain disorders include:

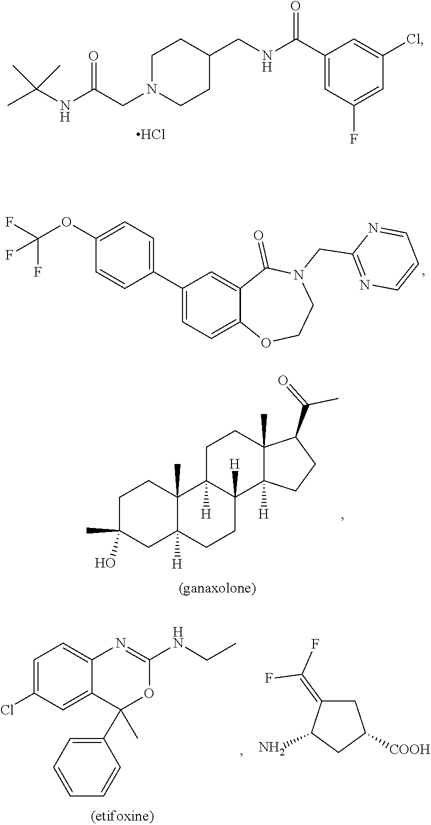

((1S,3S)-3-amino-4-(difluoromethylidene)cyclopentane-1-carboxylic acid), and pharmaceutically acceptable salts, co-crystals, tautomers, stereoisomers, solvates, hydrates, polymorphs, isotopically enriched compounds, and prodrugs thereof. Other examples of small molecules useful for the treatment of epilepsy and/or pain disorders include:

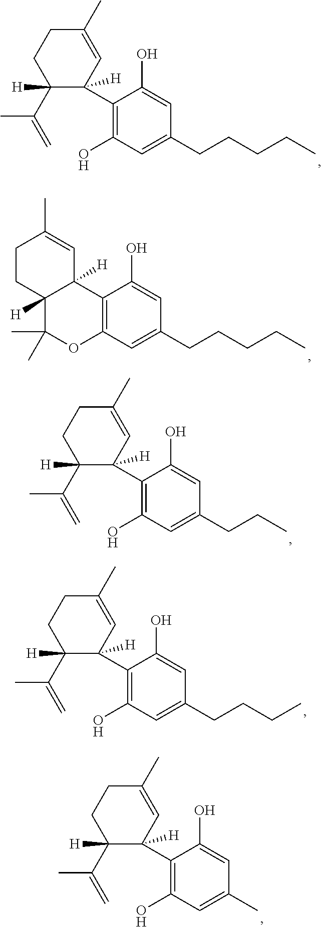

memantine, (4-benzyl-4-hydroxypiperidin-1-yl) (2,4′-bipyridin-3-yl) methanone, and pharmaceutically acceptable salts, co-crystals, tautomers, stereoisomers, solvates, hydrates, polymorphs, isotopically enriched compounds, and prodrugs thereof.

Examples of polypeptides useful for the treatment of epilepsy and/or pain disorders include antibodies, proteins, peptides (e.g., neurotensin, having an amino acid sequence pyroGlu-Leu-Tyr-Glu-Asn-Lys-Pro-Arg-Arg-Pro-Tyr-Ile-Leu-OH (SEQ ID NO: 300), or a modified form thereof, such as a peptide provided in U.S. Pat. No. 9,821,072B2, published Nov. 21, 2017, entitled “Activated neurotensin molecules and the uses thereof”, the entire contents of which are incorporated by reference in their entirety), and enzymes.

### Molecular Payloads Targeting SCN1A

The SCN1A gene, and mutations therein, are implicated in epilepsy, which predominantly affects neurons in the brain, and/or pain disorders. Modulation of SCN1A expression and activity (e.g., by suppressing the expression of SCN1A or a mutant form thereof and/or activity of a wild-type or mutant form of a protein encoded by SCN1A) therefore in some embodiments can have a therapeutic effect in subjects with epilepsy, and/or pain disorders.

### Oligonucleotides

SCN1A expression and/or activity in some embodiments can be modulated by the use of oligonucleotides targeting SCN1A sequences.

In some embodiments, an oligonucleotide useful for the treatment of epilepsy, and/or pain disorders, e.g., targeting (e.g., directly or indirectly modulating the expression or activity of) SCN1A, comprises a region of complementarity to an SCN1A transcript provided in Table 3, e.g., provided by any one of SEQ ID NOs: 546-563.

In some embodiments, examples of oligonucleotides useful for the treatment of epilepsy and/or pain disorders, e.g., targeting (e.g., directly or indirectly modulating the expression or activity of) SCN1A, are provided in International Patent Application Publication No. WO2021174036A1, published Feb. 26, 2021, entitled “Compounds and methods for modulating scn1a expression”; and U.S. Patent Application Publication No. US20180369275A1, published Dec. 27, 2018, entitled “Antisense oligomers for treatment of autosomal dominant mental retardation-5 and dravet syndrome”; the entire contents of each of which are herein incorporated by reference.

Certain oligonucleotides provided in this section may be useful in treating epilepsy and/or pain disorders by modulating the activity of genes and/or gene products other than SCN1A genes/gene products, such as SCN2A, SCN8A, SCN9A, CLN3, GRIA1, and/or PCDH19 genes/gene products. Certain oligonucleotides provided in this section may be useful in treating epilepsy and/or pain disorders by modulating the activity of genes and/or gene products other than SCN1A genes/gene products, such as GRIN2A genes/gene products.

### Polypeptides

SCN1A expression and/or activity in some embodiments can be modulated by the use of SCN1A polypeptides or polypeptides that can interact with SCN1A (e.g., to modulate its activity). In some embodiments, a polypeptide useful in the treatment of epilepsy and/or pain disorders is a peptide, a protein, an enzyme, or an antibody that modulates SCN1A, e.g., by interacting with SCN1A or a protein encoded by SCN1A.

### Small Molecules

SCN1A expression and/or activity in some embodiments can be modulated by the use of small molecules that can modulate SCN1A (e.g., to modulate its activity, or its expression).

In some embodiments, examples of small molecules useful in the treatment of epilepsy and/or pain disorders are provided in U.S. Patent Application Publication No. US20220073463A1, published Mar. 10, 2022, entitled “Formulations of t-type calcium channel modulators and methods of use thereof”; U.S. Patent Application Publication No. US20200306238A1, published Oct. 1, 2020, entitled “Treatment of CNS conditions”; U.S. Patent Application Publication No. US20190160078A1, published May 30, 2019, entitled “Ganaxolone for use in treating genetic epileptic disorders”; U.S. Patent Application Publication No. US20190091231A1, published Mar. 28, 2019, entitled “Methods of treating developmental disorders and/or seizure disorders with etifoxine”; U.S. Patent Application Publication No. US20200085838A1, published Mar. 19, 2020, entitled “Methods of treating epilepsy and neurodevelopmental disorders”; U.S. Patent Application Publication No. US20180296501A1, published Oct. 18, 2018, entitled “Methods of treating developmental encephalopathies”; and U.S. Patent Application Publication No. US20200000757A1, published Jan. 2, 2020, entitled “Methods of treating seizure disorders and prader-willi syndrome”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy and/or pain disorders by modulating the activity of genes and gene products other than SCN1A genes/gene products, such as SCN2A, SCN8A, SCN9A, CLN3, GRIA1, and/or PCDH19 genes/gene products. Certain small molecules provided in this section may be useful in treating epilepsy and/or pain disorders by modulating the activity of genes and gene products other than SCN1A genes/gene products, such as GRIN2A genes/gene products.

### Gene Therapies

SCN1A expression and/or activity in some embodiments can be modulated by the use of gene therapies that can modulate SCN1A (e.g., by delivery of nucleic acids encoding SCN1A or other molecules that interact with SCN1A).

In some embodiments, gene therapies, such as those involving administration of compounds encoding useful therapeutic agents, useful in the treatment of epilepsy and/or pain disorders are provided in U.S. Patent Application Publication No. US20190127713A1, published May 2, 2019, entitled “Crispr/cas9-based repressors for silencing gene targets in vivo and methods of use”; U.S. Patent Application Publication No. US2021162072A1, published Jun. 3, 2021, entitled “Modified adeno-associated virus vectors and delivery thereof into the central nervous system”; and International Patent Application Publication No. WO2020210542A1, published Oct. 15, 2020, entitled “Long-lasting analgesia via targeted in vivo epigenetic repression”; the entire contents of each of which are herein incorporated by reference.

Certain gene therapies provided in this section may be useful in treating epilepsy and/or pain disorders by modulating the activity of genes and gene products other than SCN1A genes/gene products, such as SCN2A, SCN8A, SCN9A, CLN3, GRIA1, and/or PCDH19 genes/gene products. Certain gene therapies provided in this section may be useful in treating epilepsy and/or pain disorders by modulating the activity of genes and gene products other than SCN1A genes/gene products, such as GRIN2A genes/gene products.

### Molecular Payloads Targeting SCN2A

The SCN2A gene, and mutations therein, are implicated in epilepsy, which predominantly affects neurons in the brain, and/or pain disorders. Modulation of SCN2A expression and activity (e.g., by suppressing the expression of SCN2A or a mutant form thereof and/or activity of a wild-type or mutant form of a protein encoded by SCN2A) therefore in some embodiments can have a therapeutic effect in subjects with epilepsy, and/or pain disorders.

### Oligonucleotides

SCN2A expression and/or activity in some embodiments can be modulated by the use of oligonucleotides targeting SCN2A sequences.

In some embodiments, an oligonucleotide useful for the treatment of epilepsy, and/or pain disorders, e.g., targeting (e.g., directly or indirectly modulating the expression or activity of) SCN2A, comprises a region of complementarity to an SCN2A transcript provided in Table 3, e.g., provided by any one of SEQ ID NOs: 564-568.

In some embodiments, examples of oligonucleotides useful for the treatment of epilepsy and/or pain disorders, e.g., targeting (e.g., directly or indirectly modulating the expression or activity of) SCN2A, are provided in International Patent Application No. WO2022032060A2, published Feb. 10, 2022, entitled “Compounds and methods for modulating scn2a”; the entire contents of which are herein incorporated by reference.

Certain oligonucleotides provided in this section may be useful in treating epilepsy and/or pain disorders by modulating the activity of genes and/or gene products other than SCN2A genes/gene products, such as SCN1A, SCN8A, SCN9A, CLN3, GRIA1, and/or PCDH19 genes/gene products. Certain oligonucleotides provided in this section may be useful in treating epilepsy and/or pain disorders by modulating the activity of genes and/or gene products other than SCN2A genes/gene products, such as GRIN2A genes/gene products.

### Polypeptides

SCN2A expression and/or activity in some embodiments can be modulated by the use of SCN2A polypeptides or polypeptides that can interact with SCN2A (e.g., to modulate its activity).

In some embodiments, examples of polypeptides (e.g., peptides; proteins, such as enzymes; antibodies; etc.) useful in the treatment of epilepsy and/or pain disorders are provided in International Patent Application Publication No. WO2020210542A1, published Oct. 15, 2020, entitled “Long-lasting analgesia via targeted in vivo epigenetic repression”; the entire contents of which are herein incorporated by reference.

Certain polypeptides provided in this section may be useful in treating epilepsy and/or pain disorders by modulating the activity of genes and gene products other than SCN2A genes/gene products, such as SCN1A, SCN8A, SCN9A, CLN3, GRIA1, and/or PCDH19 genes/gene products. Certain polypeptides provided in this section may be useful in treating epilepsy and/or pain disorders by modulating the activity of genes and gene products other than SCN2A genes/gene products, such as GRIN2A genes/gene products.

### Small Molecules

SCN2A expression and/or activity in some embodiments can be modulated by the use of small molecules that can modulate SCN2A (e.g., to modulate its activity, or its expression).

In some embodiments, examples of small molecules useful in the treatment of epilepsy and/or pain disorders are provided in U.S. Patent Application Publication No. US20220073463A1, published Mar. 10, 2022, entitled “Formulations of t-type calcium channel modulators and methods of use thereof”; U.S. Patent Application Publication No. US20200306238A1, published Oct. 1, 2020, entitled “Treatment of CNS conditions”; U.S. Patent Application Publication No. US20190160078A1, published May 30, 2019, entitled “Ganaxolone for use in treating genetic epileptic disorders”; U.S. Patent Application Publication No. US20190091231A1, published Mar. 28, 2019, entitled “Methods of treating developmental disorders and/or seizure disorders with etifoxine”; U.S. Patent Application Publication No. US20200085838A1, published Mar. 19, 2020, entitled “Methods of treating epilepsy and neurodevelopmental disorders”; and U.S. Patent Application Publication No. US20180296501A1, published Oct. 18, 2018, entitled “Methods of treating developmental encephalopathies”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy and/or pain disorders by modulating the activity of genes and gene products other than SCN2A genes/gene products, such as SCN1A, SCN8A, SCN9A, CLN3, GRIA1, and/or PCDH19 genes/gene products. Certain small molecules provided in this section may be useful in treating epilepsy and/or pain disorders by modulating the activity of genes and gene products other than SCN2A genes/gene products, such as GRIN2A genes/gene products.

### Gene Therapies

SCN2A expression and/or activity in some embodiments can be modulated by the use of gene therapies that can modulate SCN2A (e.g., by delivery of nucleic acids encoding SCN2A or other molecules that interact with SCN2A).

In some embodiments, gene therapies, such as those involving administration of compounds encoding useful therapeutic agents, useful in the treatment of epilepsy and/or pain disorders are provided in International Patent Application Publication No. WO2020210542A1, published Oct. 15, 2020, entitled “Long-lasting analgesia via targeted in vivo epigenetic repression”; the entire contents of which are herein incorporated by reference.

Certain gene therapies provided in this section may be useful in treating epilepsy and/or pain disorders by modulating the activity of genes and gene products other than SCN2A genes/gene products, such as SCN1A, SCN8A, SCN9A, CLN3, GRIA1, and/or PCDH19 genes/gene products. Certain gene therapies provided in this section may be useful in treating epilepsy and/or pain disorders by modulating the activity of genes and gene products other than SCN2A genes/gene products, such as GRIN2A genes/gene products.

### Molecular Payloads Targeting SCN8A

The SCN8A gene, and mutations therein, are implicated in epilepsy, which predominantly affects neurons in the brain, and/or pain disorders. Modulation of SCN8A expression and activity (e.g., by suppressing the expression of SCN8A or a mutant form thereof and/or activity of a wild-type or mutant form of a protein encoded by SCN8A) therefore in some embodiments can have a therapeutic effect in subjects with epilepsy, and/or pain disorders.

### Oligonucleotides

SCN8A expression and/or activity in some embodiments can be modulated by the use of oligonucleotides targeting SCN8A sequences.

In some embodiments, an oligonucleotide useful for the treatment of epilepsy, and/or pain disorders, e.g., targeting (e.g., directly or indirectly modulating the expression or activity of) SCN8A, comprises a region of complementarity to an SCN8A transcript provided in Table 3, e.g., provided by any one of SEQ ID NOs: 569-572.

In some embodiments, examples of oligonucleotides useful for the treatment of epilepsy and/or pain disorders, e.g., targeting (e.g., directly or indirectly modulating the expression or activity of) SCN8A, are provided in Lenk G M, et al. “Scn8a Antisense Oligonucleotide Is Protective in Mouse Models of SCN8A Encephalopathy and Dravet Syndrome.” Ann Neurol. (2020) 87(3):339-346; U.S. Pat. No. 11,198,874B2, published Dec. 14, 2021, entitled “SCN8A splice modulating oligonucleotides and methods of use thereof”; the entire contents of each of which are herein incorporated by reference.

Certain oligonucleotides provided in this section may be useful in treating epilepsy by modulating the activity of genes and/or gene products other than SCN8A genes/gene products, such as SCN1A, SCN2A, SCN9A, CLN3, GRIA1, and/or PCDH19 genes/gene products. Certain oligonucleotides provided in this section may be useful in treating epilepsy by modulating the activity of genes and/or gene products other than SCN8A genes/gene products, such as GRIN2A genes/gene products.

### Polypeptides

SCN8A expression and/or activity in some embodiments can be modulated by the use of SCN8A polypeptides or polypeptides that can interact with SCN8A (e.g., to modulate its activity).

In some embodiments, examples of polypeptides (e.g., peptides; proteins, such as enzymes; antibodies; etc.) useful in the treatment of epilepsy and/or pain disorders are provided in International Patent Application No. WO2020210542A1, published Oct. 15, 2020, entitled “Long-lasting analgesia via targeted in vivo epigenetic repression”; the entire contents of which are herein incorporated by reference.

Certain polypeptides provided in this section may be useful in treating epilepsy by modulating the activity of genes and gene products other than SCN8A genes/gene products, such as SCN1A, SCN2A, SCN9A, CLN3, GRIA1, and/or PCDH19 genes/gene products. Certain polypeptides provided in this section may be useful in treating epilepsy by modulating the activity of genes and gene products other than SCN8A genes/gene products, such as GRIN2A genes/gene products.

### Small Molecules

SCN8A expression and/or activity in some embodiments can be modulated by the use of small molecules that can modulate SCN8A (e.g., to modulate its activity, or its expression).

In some embodiments, examples of small molecules useful in the treatment of epilepsy and/or pain disorders are provided in U.S. Patent Application Publication No. US20220073463A1, published Mar. 10, 2022, entitled “Formulations of t-type calcium channel modulators and methods of use thereof”; U.S. Patent Application Publication No. US20200306238A1, published Oct. 1, 2020, entitled “Treatment of CNS conditions”; U.S. Patent Application Publication No. US20190160078A1, published May 30, 2019, entitled “Ganaxolone for use in treating genetic epileptic disorders”; U.S. Patent Application Publication No. US20190091231A1, published Mar. 28, 2019, entitled “Methods of treating developmental disorders and/or seizure disorders with etifoxine”; U.S. Patent Application Publication No. US20200085838A1, published Mar. 19, 2020, entitled “Methods of treating epilepsy and neurodevelopmental disorders”; and U.S. Patent Application Publication No. US20180296501A1, published Oct. 18, 2018, entitled “Methods of treating developmental encephalopathies”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy by modulating the activity of genes and gene products other than SCN8A genes/gene products, such as SCN1A, SCN2A, SCN9A, CLN3, GRIA1, and/or PCDH19 genes/gene products. Certain small molecules provided in this section may be useful in treating epilepsy by modulating the activity of genes and gene products other than SCN8A genes/gene products, such as GRIN2A genes/gene products.

### Gene Therapies

SCN8A expression and/or activity in some embodiments can be modulated by the use of gene therapies that can modulate SCN8A (e.g., by delivery of nucleic acids encoding SCN8A or other molecules that interact with SCN8A).

In some embodiments, gene therapies, such as those involving administration of compounds encoding useful therapeutic agents, useful in the treatment of epilepsy and/or pain disorders are provided in U.S. Patent Application No. US2021162072A1, published Jun. 3, 2021, entitled “Modified adeno-associated virus vectors and delivery thereof into the central nervous system”; and International Patent Application No. WO2020210542A1, published Oct. 15, 2020, entitled “Long-lasting analgesia via targeted in vivo epigenetic repression”; the entire contents of each of which are herein incorporated by reference.

Certain gene therapies provided in this section may be useful in treating epilepsy by modulating the activity of genes and gene products other than SCN8A genes/gene products, such as SCN1A, SCN2A, SCN9A, CLN3, GRIA1, and/or PCDH19 genes/gene products. Certain gene therapies provided in this section may be useful in treating epilepsy by modulating the activity of genes and gene products other than SCN8A genes/gene products, such as GRIN2A genes/gene products.

### Molecular Payloads Targeting SCN9A

The SCN9A gene, and mutations therein, are implicated in epilepsy, which predominantly affects neurons in the brain, and/or pain disorders, as well as neurodegeneration, small fiber neuropathy, nociception-related phenotypes, Alexander disease, Angelman Syndrome, autism-spectrum disorders, retinitis pigmentosa, isolated macular dystrophy, and multiple sclerosis. Modulation of SCN9A expression and activity (e.g., by suppressing the expression of SCN9A or a mutant form thereof and/or activity of a wild-type or mutant form of a protein encoded by SCN9A) therefore in some embodiments can have a therapeutic effect in subjects with epilepsy, pain disorders, and/or other neurological disorders described herein.

### Oligonucleotides

SCN9A expression and/or activity in some embodiments can be modulated by the use of oligonucleotides targeting SCN9A sequences.

In some embodiments, an oligonucleotide useful for the treatment of epilepsy, and/or pain disorders, e.g., targeting (e.g., directly or indirectly modulating the expression or activity of) SCN9A, comprises a region of complementarity to an SCN9A transcript provided in Table 3, e.g., provided by any one of SEQ ID NOs: 573-580.

In some embodiments, examples of oligonucleotides useful for the treatment of epilepsy, pain disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, e.g., targeting (e.g., directly or indirectly modulating the expression or activity of) SCN9A, are provided in U.S. Patent Application Publication No. US20210238608A1, published Aug. 5, 2021, entitled “Oligonucleotides for modulating scn9a expression”; and U.S. Patent Application Publication No. US20190218255A1, published Jul. 18, 2019, entitled “Scn9a antisense oligonucleotides”; the entire contents of each of which are herein incorporated by reference.

Certain oligonucleotides provided in this section may be useful in treating epilepsy by modulating the activity of genes and/or gene products other than SCN9A genes/gene products, such as SCN1A, SCN2A, SCN8A, CLN3, GRIA1, and/or PCDH19 genes/gene products. Certain oligonucleotides provided in this section may be useful in treating epilepsy by modulating the activity of genes and/or gene products other than SCN9A genes/gene products, such as GRIN2A genes/gene products.

### Polypeptides

SCN9A expression and/or activity in some embodiments can be modulated by the use of SCN8A polypeptides or polypeptides that can interact with SCN8A (e.g., to modulate its activity).

In some embodiments, examples of polypeptides (e.g., peptides; proteins, such as enzymes; antibodies; etc.) useful for the treatment of epilepsy, pain disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, are provided in U.S. Patent Application Publication No. US20160024208A1, published Jan. 28, 2016, entitled “Human antibodies to nav1.7”; U.S. Patent Application Publication No. US20120259096A1, published Oct. 11, 2012, entitled “Antibodies to the E1 extracellular loop of ion channels”; U.S. Patent Application Publication No. US20140073577A1, published Mar. 13, 2014, entitled “Potent and selective inhibitors of nav1.3 and nav1.7”; U.S. Pat. No. 10,344,060B2, published Jul. 9, 2019, entitled “Potent and selective inhibitors of Nav1.7”; the entire contents of each of which are herein incorporated by reference.

Certain polypeptides provided in this section may be useful in treating epilepsy by modulating the activity of genes and gene products other than SCN9A genes/gene products, such as SCN1A, SCN2A, SCN8A, CLN3, GRIA1, and/or PCDH19 genes/gene products. Certain polypeptides provided in this section may be useful in treating epilepsy by modulating the activity of genes and gene products other than SCN9A genes/gene products, such as GRIN2A genes/gene products.

### Small Molecules

SCN9A expression and/or activity in some embodiments can be modulated by the use of small molecules that can modulate SCN9A (e.g., to modulate its activity, or its expression).

In some embodiments, examples of small molecules useful for the treatment of epilepsy, pain disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, are provided in U.S. Patent Application Publication No. US20220073463A1, published Mar. 10, 2022, entitled “Formulations of t-type calcium channel modulators and methods of use thereof”; U.S. Patent Application Publication No. US20190160078A1, published May 30, 2019, entitled “Ganaxolone for use in treating genetic epileptic disorders”; U.S. Patent Application Publication No. US20190091231A1, published Mar. 28, 2019, entitled “Methods of treating developmental disorders and/or seizure disorders with etifoxine”, U.S. Patent Application Publication No. US20200085838A1, published Mar. 19, 2020, entitled “Methods of treating epilepsy and neurodevelopmental disorders”; and U.S. Patent Application Publication No. US20180296501A1, published Oct. 18, 2018, entitled “Methods of treating developmental encephalopathies”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy by modulating the activity of genes and gene products other than SCN9A genes/gene products, such as SCN1A, SCN2A, SCN8A, CLN3, GRIA1, and/or PCDH19 genes/gene products. Certain small molecules provided in this section may be useful in treating epilepsy by modulating the activity of genes and gene products other than SCN9A genes/gene products, such as GRIN2A genes/gene products.

### Gene Therapies

SCN9A expression and/or activity in some embodiments can be modulated by the use of gene therapies that can modulate SCN9A (e.g., by delivery of nucleic acids encoding SCN9A or other molecules that interact with SCN9A).

In some embodiments, gene therapies, such as those involving administration of compounds encoding useful therapeutic agents, useful for the treatment of epilepsy, pain disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, are provided in U.S. Patent Application Publication No. US2021162072A1, published Jun. 3, 2021, entitled “Modified adeno-associated virus vectors and delivery thereof into the central nervous system”; International Patent Application Publication No. WO2020210542A1, published Oct. 15, 2020, entitled “Long-lasting analgesia via targeted in vivo epigenetic repression”; the entire contents of each of which are herein incorporated by reference.

Certain gene therapies provided in this section may be useful in treating epilepsy by modulating the activity of genes and gene products other than SCN9A genes/gene products, such as SCN1A, SCN2A, SCN8A, CLN3, GRIA1, and/or PCDH19 genes/gene products. Certain gene therapies provided in this section may be useful in treating epilepsy by modulating the activity of genes and gene products other than SCN9A genes/gene products, such as GRIN2A genes/gene products.

### Molecular Payloads Targeting CLN3

The CLN3 gene, and mutations therein, are implicated in epilepsy, which predominantly affects neurons in the brain, and/or pain disorders. Modulation of CLN3 expression and activity (e.g., by suppressing the expression of CLN3 or a mutant form thereof and/or activity of a wild-type or mutant form of a protein encoded by CLN3) therefore in some embodiments can have a therapeutic effect in subjects with epilepsy, and/or pain disorders.

### Oligonucleotides

CLN3 expression and/or activity in some embodiments can be modulated by the use of oligonucleotides targeting CLN3 sequences.

In some embodiments, an oligonucleotide useful for the treatment of epilepsy, and/or pain disorders, e.g., targeting (e.g., directly or indirectly modulating the expression or activity of) CLN3, comprises a region of complementarity to a CLN3 transcript provided in Table 3, e.g., provided by any one of SEQ ID NOs: 581-586.

In some embodiments, examples of oligonucleotides useful for the treatment of epilepsy and/or pain disorders, e.g., targeting (e.g., directly or indirectly modulating the expression or activity of) CLN3, are provided in International Patent Application Publication No. WO2020055917A1, published Mar. 19, 2020, entitled “Compounds and methods for modulating cln3 expression”; the entire contents of which are herein incorporated by reference.

Certain oligonucleotides provided in this section may be useful in treating epilepsy by modulating the activity of genes and/or gene products other than CLN3 genes/gene products, such as SCN1A, SCN2A, SCN8A, SCN9A, GRIA1, and/or PCDH19 genes/gene products. Certain oligonucleotides provided in this section may be useful in treating epilepsy by modulating the activity of genes and/or gene products other than CLN3 genes/gene products, such as GRIN2A genes/gene products.

### Polypeptides

CLN3 expression and/or activity in some embodiments can be modulated by the use of CLN3 polypeptides or polypeptides that can interact with CLN3 (e.g., to modulate its activity).

In some embodiments, examples of polypeptides (e.g., peptides; proteins, such as enzymes; antibodies; etc.) useful in the treatment of epilepsy and/or pain disorders are provided in Nelson T, et al. “Lack of specificity of antibodies raised against CLN3, the lysosomal/endosomal transmembrane protein mutated in juvenile Batten disease.” Biosci Rep. (2017) 37(6):BSR20171229; the entire contents of which are herein incorporated by reference.

Certain polypeptides provided in this section may be useful in treating epilepsy by modulating the activity of genes and gene products other than CLN3 genes/gene products, such as SCN1A, SCN2A, SCN8A, SCN9A, GRIA1, and/or PCDH19 genes/gene products. Certain polypeptides provided in this section may be useful in treating epilepsy by modulating the activity of genes and gene products other than CLN3 genes/gene products, such as GRIN2A genes/gene products.

### Small Molecules

CLN3 expression and/or activity in some embodiments can be modulated by the use of small molecules that can modulate CLN3 (e.g., to modulate its activity, or its expression).

In some embodiments, examples of small molecules useful in the treatment of epilepsy and/or pain disorders are provided in U.S. Patent Application Publication No. US20190160078A1, published May 30, 2019, entitled “Ganaxolone for use in treating genetic epileptic disorders”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy by modulating the activity of genes and gene products other than CLN3 genes/gene products, such as SCN1A, SCN2A, SCN8A, SCN9A, GRIA1, and/or PCDH19 genes/gene products. Certain small molecules provided in this section may be useful in treating epilepsy by modulating the activity of genes and gene products other than CLN3 genes/gene products, such as GRIN2A genes/gene products.

### Gene Therapies

CLN3 expression and/or activity in some embodiments can be modulated by the use of gene therapies that can modulate CLN3 (e.g., by delivery of nucleic acids encoding CLN3 or other molecules that interact with CLN3).

In some embodiments, gene therapies, such as those involving administration of a compounds encoding useful therapeutic agents, useful in the treatment of epilepsy and/or pain disorders are provided in U.S. Patent Application Publication No. US2021162072A1, published Jun. 3, 2021, entitled “Modified adeno-associated virus vectors and delivery thereof into the central nervous system”; the entire contents of each of which are herein incorporated by reference.

Certain gene therapies provided in this section may be useful in treating epilepsy by modulating the activity of genes and gene products other than CLN3 genes/gene products, such as SCN1A, SCN2A, SCN8A, SCN9A, GRIA1, and/or PCDH19 genes/gene products. Certain gene therapies provided in this section may be useful in treating epilepsy by modulating the activity of genes and gene products other than CLN3 genes/gene products, such as GRIN2A genes/gene products.

### Molecular Payloads Targeting GRIA1

The GRIA1 gene, and mutations therein, are implicated in epilepsy, which predominantly affects neurons in the brain, and/or pain disorders. Modulation of GRIA1 expression and activity (e.g., by suppressing the expression of GRIA1 or a mutant form thereof and/or activity of a wild-type or mutant form of a protein encoded by GRIA1) therefore in some embodiments can have a therapeutic effect in subjects with epilepsy, and/or pain disorders.

### Oligonucleotides

GRIA1 expression and/or activity in some embodiments can be modulated by the use of oligonucleotides targeting GRIA1 sequences.

In some embodiments, an oligonucleotide useful for the treatment of epilepsy, and/or pain disorders, e.g., targeting (e.g., directly or indirectly modulating the expression or activity of) GRIA1, comprises a region of complementarity to a GRIA1 transcript provided in Table 3, e.g., provided by any one of SEQ ID NOs: 587-600.

In some embodiments, examples of oligonucleotides useful for the treatment of epilepsy and/or pain disorders, e.g., targeting (e.g., directly or indirectly modulating the expression or activity of) GRIA1, are provided in Zheng Z, et al. “Two-stage AMPA receptor trafficking in classical conditioning and selective role for glutamate receptor subunit 4 (tGluA4) flop splice variant.” J Neurophysiol. (2012) 108(1):101-11; and Hu Z, et al. “miR-501-3p mediates the activity-dependent regulation of the expression of AMPA receptor subunit GluA1.” J Cell Biol. (2015) 208(7):949-59; the entire contents of each of which are herein incorporated by reference.

Certain oligonucleotides provided in this section may be useful in treating epilepsy by modulating the activity of genes and/or gene products other than GRIA1 genes/gene products, such as SCN1A, SCN2A, SCN8A, SCN9A, CLN3, and/or PCDH19 genes/gene products. Certain oligonucleotides provided in this section may be useful in treating epilepsy by modulating the activity of genes and/or gene products other than GRIA1 genes/gene products, such as GRIN2A genes/gene products.

### Polypeptides

GRIA1 expression and/or activity in some embodiments can be modulated by the use of GRIA1 polypeptides or polypeptides that can interact with GRIA1 (e.g., to modulate its activity).

In some embodiments, examples of polypeptides (e.g., peptides; proteins, such as enzymes; antibodies; etc.) useful in the treatment of epilepsy are provided in Japanese Patent Application Publication No. JP2016030733A, published Dec. 5, 2018, entitled “Monoclonal antibody that recognizes AMPA type glutamic acid receptor subunit and use thereof”; the entire contents of which are herein incorporated by reference.

Certain polypeptides provided in this section may be useful in treating epilepsy by modulating the activity of genes and gene products other than GRIA1 genes/gene products, such as SCN1A, SCN2A, SCN8A, SCN9A, CLN3, and/or PCDH19 genes/gene products. Certain polypeptides provided in this section may be useful in treating epilepsy by modulating the activity of genes and gene products other than GRIA1 genes/gene products, such as GRIN2A genes/gene products.

### Small Molecules

GRIA1 expression and/or activity in some embodiments can be modulated by the use of small molecules that can modulate GRIA1 (e.g., to modulate its activity, or its expression).

In some embodiments, examples of small molecules useful in the treatment of epilepsy are provided in Kim J E, et al. “PTEN Is Required for The Anti-Epileptic Effects of AMPA Receptor Antagonists in Chronic Epileptic Rats.” Int J Mol Sci. (2020) 21(16):5643; U.S. Patent Application Publication No. US20170083664A1, published Mar. 23, 2017, entitled “Methods of Diagnosing and Treating Autism”; and U.S. Patent Application Publication No. US20150125441A1, published Jul. 7, 2020, entitled “Methods of treating depression and pain”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy by modulating the activity of genes and gene products other than GRIA1 genes/gene products, such as SCN1A, SCN2A, SCN8A, SCN9A, CLN3, and/or PCDH19 genes/gene products. Certain small molecules provided in this section may be useful in treating epilepsy by modulating the activity of genes and gene products other than GRIA1 genes/gene products, such as GRIN2A genes/gene products.

### Gene Therapies

GRIA1 expression and/or activity in some embodiments can be modulated by the use of gene therapies that can modulate GRIA1 (e.g., by delivery of nucleic acids encoding GRIA1 or other molecules that interact with GRIA1).

In some embodiments, gene therapies, such as those involving administration of compounds encoding useful therapeutic agents, useful in the treatment of epilepsy are provided in U.S. Patent Application Publication No. US2021162072A1, published Jun. 3, 2021, entitled “Modified adeno-associated virus vectors and delivery thereof into the central nervous system”; the entire contents of each of which are herein incorporated by reference.

Certain gene therapies provided in this section may be useful in treating epilepsy by modulating the activity of genes and gene products other than GRIA1 genes/gene products, such as SCN1A, SCN2A, SCN8A, SCN9A, CLN3, and/or PCDH19 genes/gene products. Certain gene therapies provided in this section may be useful in treating epilepsy by modulating the activity of genes and gene products other than GRIA1 genes/gene products, such as GRIN2A genes/gene products.

### Molecular Payloads Targeting GRIN2A

The GRIN2A gene, and mutations therein, are implicated in epilepsy, which predominantly affects neurons in the brain, and/or pain disorders. Modulation of GRIN2A expression and activity (e.g., by suppressing the expression of GRIN2A or a mutant form thereof and/or activity of a wild-type or mutant form of a protein encoded by GRIN2A) therefore in some embodiments can have a therapeutic effect in subjects with epilepsy, and/or pain disorders.

### Oligonucleotides

GRIN2A expression and/or activity in some embodiments can be modulated by the use of oligonucleotides targeting GRIN2A sequences.

In some embodiments, an oligonucleotide useful for the treatment of epilepsy, and/or pain disorders, e.g., targeting (e.g., directly or indirectly modulating the expression or activity of) GRIN2A, comprises a region of complementarity to a GRIN2A transcript provided in Table 3, e.g., provided by any one of SEQ ID NOs: 838-840.

In some embodiments, examples of oligonucleotides useful for the treatment of epilepsy and/or pain disorders, e.g., targeting (e.g., directly or indirectly modulating the expression or activity of) GRIN2A, are provided in US20210268667A1, published Sep. 2, 2021, entitled “Antisense oligomers for treatment of non-sense mediated ma decay based conditions and diseases”; the entire contents of which are herein incorporated by reference.

Certain oligonucleotides provided in this section may be useful in treating epilepsy and/or pain disorders by modulating the activity of genes and/or gene products other than GRIN2A genes/gene products, such as other genes/gene products associated with epilepsy and/or pain disorders.

### Polypeptides

GRIN2A expression and/or activity in some embodiments can be modulated by the use of GRIN2A polypeptides or polypeptides that can interact with GRIN2A (e.g., to modulate its activity). In some embodiments, a polypeptide useful in the treatment of epilepsy and/or pain disorders is a peptide, a protein, an enzyme, or an antibody that modulates GRIN2A, e.g., by interacting with GRIN2A or a protein encoded by GRIN2A.

### Small Molecules

GRIN2A expression and/or activity in some embodiments can be modulated by the use of small molecules that can modulate GRIN2A (e.g., to modulate its activity, or its expression).

In some embodiments, examples of small molecules useful in the treatment of epilepsy and/or pain disorders are provided in US20220008355A1, published Jan. 13, 2022, entitled “Use of cannabinolids in the treatment of epilepsy”; US20200368181A1, published Nov. 26, 2020, entitled “Methods of treating anti-nmdar-associated neuropsychiatric disorders”; U.S. Ser. No. 11/285,139B2, published Mar. 29, 2022, entitled “Treatment of CNS conditions”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy and/or pain disorders by modulating the activity of genes and/or gene products other than GRIN2A genes/gene products, such as other genes/gene products associated with epilepsy and/or pain disorders.

### Gene Therapies

GRIN2A expression and/or activity in some embodiments can be modulated by the use of gene therapies that can modulate GRIN2A (e.g., by delivery of nucleic acids encoding GRIN2A or other molecules that interact with GRIN2A).

In some embodiments, gene therapies, such as those involving administration of a compounds encoding useful therapeutic agents, useful in the treatment of epilepsy and/or pain disorders include payloads which encode GluN2A protein or functional fragments thereof.

Certain gene therapies provided in this section may be useful in treating epilepsy and/or pain disorders by modulating the activity of genes and/or gene products other than GRIN2A genes/gene products, such as other genes/gene products associated with epilepsy and/or pain disorders.

### Molecular Payloads Targeting PCDH19

The PCDH19 gene, and mutations therein, are implicated in epilepsy, which predominantly affects neurons in the brain, and/or pain disorders. Modulation of PCDH19 expression and activity (e.g., by suppressing the expression of PCDH19 or a mutant form thereof and/or activity of a wild-type or mutant form of a protein encoded by PCDH19) therefore in some embodiments can have a therapeutic effect in subjects with epilepsy, and/or pain disorders.

### Oligonucleotides

PCDH19 expression and/or activity in some embodiments can be modulated by the use of oligonucleotides targeting PCDH19 sequences.

In some embodiments, an oligonucleotide useful for the treatment of epilepsy and/or pain disorders, e.g., targeting (e.g., directly or indirectly modulating the expression or activity of) PCDH19, comprises a region of complementarity to a PCDH19 transcript provided in Table 3, e.g., provided by any one of SEQ ID NOs: 601-604.

Certain oligonucleotides provided in this section may be useful in treating epilepsy by modulating the activity of genes and/or gene products other than PCDH19 genes/gene products, such as SCN1A, SCN2A, SCN8A, SCN9A, CLN3, and/or GRIA1 genes/gene products. Certain oligonucleotides provided in this section may be useful in treating epilepsy by modulating the activity of genes and/or gene products other than PCDH19 genes/gene products, such as GRIN2A genes/gene products.

### Polypeptides

PCDH19 expression and/or activity in some embodiments can be modulated by the use of PCDH19 polypeptides or polypeptides that can interact with PCDH19 (e.g., to modulate its activity). In some embodiments, a polypeptide useful in the treatment of epilepsy and/or pain disorders is a peptide, a protein, an enzyme, or an antibody that modulates PCDH19, e.g., by interacting with PCDH19 or a protein encoded by PCDH19.

### Small Molecules

PCDH19 expression and/or activity in some embodiments can be modulated by the use of small molecules that can modulate PCDH19 (e.g., to modulate its activity, or its expression).

In some embodiments, examples of small molecules useful in the treatment of epilepsy are provided in U.S. Patent Application Publication No. US20220073463A1, published Mar. 10, 2022, entitled “Formulations of t-type calcium channel modulators and methods of use thereof”; U.S. Patent Application Publication No. US20200306238A1, published Oct. 1, 2020, entitled “Treatment of CNS conditions”; International Patent Application Publication No. WO2020231837A1, published Nov. 19, 2020, entitled “Pharmaceutical composition containing brexanolone, ganaxolone, or zuranolone, and use thereof”; U.S. Patent Application Publication No. US20190160078A1, published May 30, 2019, entitled “Ganaxolone for use in treating genetic epileptic disorders”; U.S. Patent Application Publication No. US20190091231A1, published Mar. 28, 2019, entitled “Methods of treating developmental disorders and/or seizure disorders with etifoxine”; U.S. Patent Application Publication No. US20200085838A1, published Mar. 19, 2020, entitled “Methods of treating epilepsy and neurodevelopmental disorders”; U.S. Patent Application Publication No. US20180296501A1, published Oct. 18, 2018, entitled “Methods of treating developmental encephalopathies”; and U.S. Patent Application Publication No. US20200000757A1, published Jan. 2, 2020, entitled “Methods of treating seizure disorders and prader-willi syndrome”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy by modulating the activity of genes and gene products other than PCDH19 genes/gene products, such as SCN1A, SCN2A, SCN8A, SCN9A, CLN3, and/or GRIA1 genes/gene products. Certain small molecules provided in this section may be useful in treating epilepsy by modulating the activity of genes and gene products other than PCDH19 genes/gene products, such as GRIN2A genes/gene products.

### Gene Therapies

PCDH19 expression and/or activity in some embodiments can be modulated by the use of gene therapies that can modulate PCDH19 (e.g., by delivery of nucleic acids encoding PCDH19 or other molecules that interact with PCDH19).

In some embodiments, gene therapies, such as those involving administration of compounds encoding useful therapeutic agents, useful in the treatment of epilepsy are provided in U.S. Patent Application Publication No. US2021162072A1, published Jun. 3, 2021, entitled “Modified adeno-associated virus vectors and delivery thereof into the central nervous system”; the entire contents of which are herein incorporated by reference.

Certain gene therapies provided in this section may be useful in treating epilepsy by modulating the activity of genes and gene products other than PCDH19 genes/gene products, such as SCN1A, SCN2A, SCN8A, SCN9A, CLN3, and/or GRIA1 genes/gene products. Certain gene therapies provided in this section may be useful in treating epilepsy by modulating the activity of genes and gene products other than PCDH19 genes/gene products, such as GRIN2A genes/gene products.

### Molecular Payloads for the Treatment of Dravet Syndrome

Various molecular payloads may be useful in the treatment of Dravet syndrome, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of Dravet syndrome may include, in some embodiments, molecular payloads which modulate (e.g., increase or decrease) expression or activity of SCN1A. In particular, molecular payloads which increase expression of SCN1A (e.g., resulting in increased levels of functional sodium voltage-gated channel alpha subunit 1 or a functional fragment thereof) in some embodiments are useful in treatment of Dravet syndrome. See, e.g., Strzelczyk, et al., “Therapeutic advances in Dravet syndrome: a targeted literature review” Exp Rev Neurotherapeutics 20(10):1065-1079 (2020), the entire contents of which are herein incorporated by reference.

Examples of oligonucleotides useful for the treatment of Dravet syndrome, e.g., oligonucleotides targeting (e.g., directly or indirectly modulating the expression or activity of) genes associated with Dravet syndrome (e.g., SCN1A), include those listed in Table 16 below. Each oligonucleotide provided in Table 16 may have any modification pattern disclosed herein.

TABLE 16
Oligonucleotides for the treatment of Dravet syndrome

| | | Oligo- | |
|---|---|---|---|
| SEQ | | nucleotide | |
| ID | Target | Sequence | |
| NO: | gene | (5′-3′) | Oligonucleotide Structure (5′-3′) |
| 181 | SCN1A | AGTTGGAGCA | Ans Gns Tns Tns Gns Gns Ans Gns mCns Ans Ans Gns |
| | | AGATTATC | Ans Tns Tns Ans Tns mCn |
| 182 | SCN1A | AGTTGGAGCA | Aes Geo Teo Tes Ges Ges Aes Ges mCes Aes Aes Ges |
| | | AGATTATC | Aes Tes Tes Aes Tes mCe |
| 183 | SCN1A | AGTTGGAGCA | Aes Tes Tes Teo Ges Ges Aes Ges mCes Aes Aes Ges |
| | | AGATTATC | Aes Geo Tes Aes Tes mCe |
| 184 | SCN1A | AGTTGGAGCA | Aes Tes Tes Tes Ges Geo Aes Ges mCes Aes Aes Ges |
| | | AGATTATC | Aes Geo Tes Aes Tes mCe |
| 185 | SCN1A | AGTTGGAGCA | Aes Geo Tes Tes Ges Ges Aes Geo mCes Aes Aes Ges |
| | | AGATTATC | Aes Tes Tes Aes Tes mCe |
| 186 | SCN1A | AGTTGGAGCA | Aes Ges Tes Teo Geo Ges Aes Ges mCes Aes Aes Ges |
| | | AGATTATC | Aes Tes Tes Aes Tes mCe |
| 187 | SCN1A | AGTTGGAGCA | Aes Ges Tes Tes Ges Ges Aes Geo mCeo Aes Aes Ges |
| | | AGATTATC | Aes Tes Tes Aes Tes mCe |
| 188 | SCN1A | AGTTGGAGCA | Aes Ges Tes Aes Ges Ges Aes Ges mCes Aeo Aeo Ges |
| | | AGATTATC | Aes Tes Tes Tes Tes mCe |
| 189 | SCN1A | AGTTGGAGCA | Aes Ges Tes Tes Ges mCe Aes Ges mCes Aes Aes Geo |
| | | AGATTATC | Aeo Tes Tes Aes Tes Ges |
| 190 | SCN1A | AGTTGGAGCA | Aes Geo Tes Tes Ges Ges Aes Ges mCes Aeo Aes Ges |
| | | AGATTATC | Aes Tes Tes Aes Tes mCe |
| 191 | SCN1A | AGTTGGAGCA | Aes Geo Tes Tes Ges Ges Aes Ges mCes Aes Aes Geo |
| | | AGATTATC | Aes Tes Tes Aes Tes mCe |
| 192 | SCN1A | AGTTGGAGCA | Aes Geo Tes Aes Ges Ges Aes Ges mCes Aes Aes Ges |
| | | AGATTATC | Aes Tes Teo Tes Tes mCe |
| 193 | SCN1A | AGTTGGAGCA | Aes Ges Teo Tes Ges Ges Aes Ges mCes Aes Aes Ges |
| | | AGATTATC | Aes Tes Teo Aes Tes mCe |
| 194 | SCN1A | AGTTGGAGCA | Aes Ges Tes Tes Geo Ges Aes Ges mCes Aes Aes Ges |
| | | AGATTATC | Aes Tes Teo Aes Tes mCe |
| 195 | SCN1A | AGTTGGAGCA | Aes Ges Tes Tes Ges Geo Aeo Ges mCes Aes Aes Ges |
| | | AGATTATC | Aes Tes Tes Aes Tes mCe |
| 196 | SCN1A | AGTTGGAGCA | Aes Ges Tes Tes Ges Ges Aeo Ges mCes Aes Aes Ges |
| | | AGATTATC | Aes Tes Teo Aes Tes mCe |
| 197 | SCN1A | AGTTGGAGCA | Aes Teo Tes Aes Ges Ges Aes Ges mCeo Aes Aes Ges |
| | | AGATTATC | Aes Ges Tes Tes Tes mCe |
| 198 | SCN1A | AGTTGGAGCA | Aes Ges Tes Tes Ges Ges Aes Ges mCeo Aes Aes Ges |
| | | AGATTATC | Aes Tes Teo Aes Tes mCe |
| 199 | SCN1A | AGTTGGAGCA | Aes Ges Tes Tes Ges Ges Aes Ges mCes Aes Aeo Ges |
| | | AGATTATC | Aes Tes Teo Aes Tes mCe |
| 200 | SCN1A | AGTTGGAGCA | Aes Ges Tes Tes Ges Ges Aes Ges mCes Aes Aes Geo |
| | | AGATTATC | Aes Teo Tes Aes Tes mCe |
| 201 | SCN1A | AGTTGGAGCA | Aes Ges Tes Tes Ges mCe Aes Ges mCes Aes Aes Ges |
| | | AGATTATC | Aeo Tes Teo Aes Tes Ges |
| 202 | SCN1A | AGTTGGAGCA | Aes Teo Teo Aes Ges Ges Aes Ges mCes Aes Aes Ges |
| | | AGATTATC | Aes Ges Tes Tes Tes mCe |
| 203 | SCN1A | AAGTTGGAGC | Aeo Aes Ges Tes Tes Ges Ges Aes Ges mCes Aes Aes |
| | | AAGATTATCC | Ges Aes Tes Tes Aes Tes mCeo mCe |
| 204 | SCN1A | AGTTGGAGCA | Aes Ges Tes Tes Ges Ges Aes Ges mCes Aes Aes Ges |
| | | AGATTATCC | Aes Tes Tes Aes Tes mCeo mCe |
| 205 | SCN1A | AAGTTGGAGC | Aeo Aes Ges Tes Tes Ges Ges Aes Ges mCes Aes Aes |
| | | AAGATTATC | Ges Aes Tes Tes Aes Tes mCe |
| 206 | SCN1A | GGTAGCAAAA | Gns Gns Tns Ans Gns mCns Ans Ans Ans Ans Gns Gns |
| | | GGGGTAAT | Gns Gns Tns Ans Ans Tn |
| 207 | SCN1A | AGCAAAAGGG | Ans Gns mCns Ans Ans Ans Ans Gns Gns Gns Gns Tns |
| | | GTAATACA | Ans Ans Tns Ans mCns An |
| 208 | SCN1A | GCAAAAGGGG | Gns mCns Ans Ans Ans Ans Gns Gns Gns Gns Tns Ans |
| | | TAATACAG | Ans Tns Ans mCns Ans Gn |
| 209 | SCN1A | CAAAAGGGGT | mCns Ans Ans Ans Ans Gns Gns Gns Gns Tns Ans Ans |
| | | AATACAGT | Tns Ans mCns Ans Gns Tn |
| 210 | SCN1A | AAAGGGGTAA | Ans Ans Ans Gns Gns Gns Gns Tns Ans Ans Tns Ans |
| | | TACAGTAC | mCns Ans Gns Tns Ans mCn |
| 211 | SCN1A | AAGGGGTAAT | Ans Ans Gns Gns Gns Gns Tns Ans Ans Tns Ans mCns |
| | | ACAGTACC | Ans Gns Tns Ans mCns mCn |
| 212 | SCN1A | GTAATACAGT | Gns Tns Ans Ans Tns Ans mCns Ans Gns Tns Ans mCns |
| | | ACCCATAA | mCns mCns Ans Tns Ans An |
| 213 | SCN1A | TAATACAGTA | Tns Ans Ans Tns Ans mCns Ans Gns Tns Ans mCns |
| | | CCCATAAT | mCns mCns Ans Tns Ans Ans Tn |
| 214 | SCN1A | AATACAGTAC | Ans Ans Tns Ans mCns Ans Gns Tns Ans mCns mCns |
| | | CCATAATA | mCns Ans Tns Ans Ans Tns An |
| 215 | SCN1A | ATACAGTACC | Ans Tns Ans mCns Ans Gns Tns Ans mCns mCns mCns |
| | | CATAATAA | Ans Tns Ans Ans Tns Ans An |
| 216 | SCN1A | TACAGTACCC | Tns Ans mCns Ans Gns Tns Ans mCns mCns mCns Ans |
| | | ATAATAAA | Tns Ans Ans Tns Ans Ans An |
| 217 | SCN1A | ACAGTACCCA | Ans mCns Ans Gns Tns Ans mCns mCns mCns Ans Tns |
| | | TAATAAAG | Ans Ans Tns Ans Ans Ans Gn |
| 218 | SCN1A | CCCATCCAAG | mCns mCns mCns Ans Tns mCns mCns Ans Ans Gns Tns |
| | | TTGGAGCA | Tns Gns Gns Ans Gns mCns An |
| 219 | SCN1A | CCATCCAAGT | mCns mCns Ans Tns mCns mCns Ans Ans Gns Tns Tns |
| | | TGGAGCAA | Gns Gns Ans Gns mCns Ans An |
| 220 | SCN1A | CATCCAAGTT | mCns Ans Tns mCns mCns Ans Ans Gns Tns Tns Gns |
| | | GGAGCAAG | Gns Ans Gns mCns Ans Ans Gn |
| 221 | SCN1A | ATCCAAGTTG | Ans Tns mCns mCns Ans Ans Gns Tns Tns Gns Gns Ans |
| | | GAGCAAGA | Gns mCns Ans Ans Gns An |
| 222 | SCN1A | TCCAAGTTGG | Tns mCns mCns Ans Ans Gns Tns Tns Gns Gns Ans Gns |
| | | AGCAAGAT | mCns Ans Ans Gns Ans Tn |
| 223 | SCN1A | CCAAGTTGGA | mCns mCns Ans Ans Gns Tns Tns Gns Gns Ans Gns |
| | | GCAAGATT | mCns Ans Ans Gns Ans Tns Tn |
| 224 | SCN1A | CAAGTTGGAG | mCns Ans Ans Gns Tns Tns Gns Gns Ans Gns mCns Ans |
| | | CAAGATTA | Ans Gns Ans Tns Tns An |
| 225 | SCN1A | AAGTTGGAGC | Ans Ans Gns Tns Tns Gns Gns Ans Gns mCns Ans Ans |
| | | AAGATTAT | Gns Ans Tns Tns Ans Tn |
| 226 | SCN1A | TTGGAGCAAG | Tns Tns Gns Gns Ans Gns mCns Ans Ans Gns Ans Tns |
| | | ATTATCCT | Tns Ans Tns mCns mCns Tn |
| 227 | SCN1A | TGGAGCAAGA | Tns Gns Gns Ans Gns mCns Ans Ans Gns Ans Tns Tns |
| | | TTATCCTA | Ans Tns mCns mCns Tns An |
| 228 | SCN1A | GGAGCAAGAT | Gns Gns Ans Gns mCns Ans Ans Gns Ans Tns Tns Ans |
| | | TATCCTAT | Tns mCns mCns Tns Ans Tn |
| 229 | SCN1A | GAGCAAGATT | Gns Ans Gns mCns Ans Ans Gns Ans Tns Tns Ans Tns |
| | | ATCCTATA | mCns mCns Tns Ans Tns An |

Abbreviations used in Table 16:
mC = 5-methyl cytosine; n = 2′-NMA sugar moiety; s = phosphorothioate internucleoside linkage; o = phosphodiester internucleoside linkage; s = phosphorothioate internucleoside linkage; x = internucleoside linkage that is either phosphorothioate or phosphodiester; e = 2′-MOE modified sugar. In each sequence listed in Table 16, each T may be optionally and independently replaced with a U, and each U may be optionally and independently replaced with a T.

Examples of small molecules useful for the treatment of Dravet syndrome include: milnacipran, torasemide, resperidone, pinacidil, benidipine, ketoconazole, ebselen, tadalafil, zeranol, nefazadone, lomerizine, icariin, omeprazole, L-694,247, nitrendipine, nimetazepam, amlexanox, mosapride, sertraline or stanozolol, oxadiazoles (e.g., 3-[5-(2-fluoro-phenyl)-[1,2,4]oxadiazol-3-yl]benzoic acid), fenfluramine, stiripentol, topiramate, bromide, valproate, clobazam, cannabidiol, soticlestat, ataluren, verapamil, clemizole,

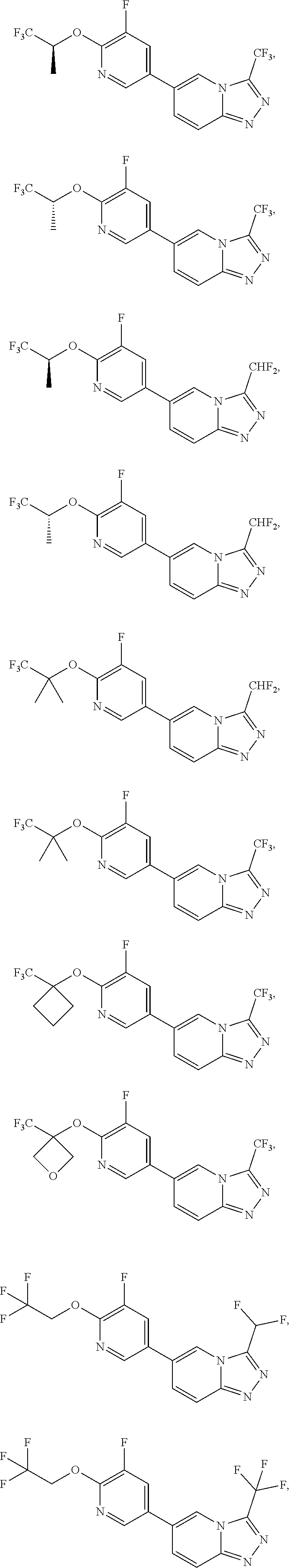

and pharmaceutically acceptable salts, co-crystals, tautomers, stereoisomers, solvates, hydrates, polymorphs, isotopically enriched compounds, and prodrugs thereof.

Examples of polypeptides useful for the treatment of Dravet syndrome include antibodies, proteins, peptides, and enzymes. In some embodiments, a polypeptide useful for the treatment of Dravet syndrome comprises an amino acid sequence having at least 70% sequence identity (e.g., at least 70%, 75%, 80%, 85%, 90%, 95%, 96%, 97%, 98%, 99% or 100%) to the amino acid sequence:

(SEQ ID NO: 808)
MEQTVLVPPGPDSFNFFTRESLAAIERRIAEEKAKNPKPDKKDDDENGPKPNSDLEAGKNLPFIYGDIPP
EMVSEPLEDLDPYYINKKTFIVLNKGKAIFRFSATSALYILTPFNPLRKIAIKILVHSLFSMLIMCTILTNCVFMT
MSNPPDWTKNVEYTFTGIYTFESLIKIIARGFCLEDFTFLRDPWNWLDFTVITFAYVTEFVDLGNVSALRTFRVLR
ALKTISVIPGLKTIVGALIQSVKKLSDVMILTVFCLSVFALIGLQLFMGNLRNKCIQWPPTNASLEEHSIEKNITV
NYNGTLINETVFEFDWKSYIQDSRYHYFLEGFLDALLCGNSSDAGQCPEGYMCVKAGRNPNYGYTSFDTFSWAFLS
LFRLMTQDFWENLYQLTLRAAGKTYMIFFVLVIFLGSFYLINLILAVVAMAYEEQNQATLEEAEQKEAEFQQMIEQ
LKKQQEAAQQAATATASEHSREPSAAGRLSDSSSEASKLSSKSAKERRNRRKKRKQKEQSGGEEKDEDEFQKSESE
DSIRRKGFRFSIEGNRLTYEKRYSSPHQSLLSIRGSLFSPRRNSRTSLFSFRGRAKDVGSENDFADDEHSTFEDNE
SRRDSLFVPRRHGERRNSNLSQTSRSSRMLAVFPANGKMHSTVDCNGVVSLVGGPSVPTSPVGQLLPEVIIDKPAT
DDNGTTTETEMRKRRSSSFHVSMDFLEDPSQRQRAMSIASILINTVEELEESRQKCPPCWYKFSNIFLIWDCSPYW
LKVKHVVNLVVMDPFVDLAITICIVLNTLFMAMEHYPMTDHFNNVLTVGNLVFTGIFTAEMFLKIIAMDPYYYFQE
GWNIFDGFIVTLSLVELGLANVEGLSVLRSFRLLRVFKLAKSWPTLNMLIKIIGNSVGALGNLTLVLAIIVFIFAV
VGMQLFGKSYKDCVCKIASDCQLPRWHMNDFFHSFLIVFRVLCGEWIETMWDCMEVAGQAMCLTVFMMVMVIGNLV
VLNLFLALLLSSFSADNLAATDDDNEMNNLQIAVDRMHKGVAYVKRKIYEFIQQSFIRKQKILDEIKPLDDLNNKK
DSCMSNHTAEIGKDLDYLKDVNGTTSGIGTGSSVEKYIIDESDYMSFINNPSLTVTVPIAVGESDFENLNTEDFSS
ESDLEESKEKLNESSSSSEGSTVDIGAPVEEQPVVEPEETLEPEACFTEGCVQRFKCCQINVEEGRGKQWWNLRRT
CFRIVEHNWFETFIVEMILLSSGALAFEDIYIDQRKTIKTMLEYADKVETYIFILEMLLKWVAYGYQTYFTNAWCW
LDFLIVDVSLVSLTANALGYSELGAIKSLRTLRALRPLRALSRFEGMRVVVNALLGAIPSIMNVLLVCLIFWLIFS
IMGVNLFAGKFYHCINTTTGDRFDIEDVNNHTDCLKLIERNETARWKNVKVNFDNVGFGYLSLLQVATFKGWMDIM
YAAVDSRNVELQPKYEESLYMYLYFVIFIIFGSFFTLNLFIGVIIDNFNQQKKKFGGQDIFMTEEQKKYYNAMKKL
GSKKPQKPIPRPGNKFQGMVFDFVTRQVEDISIMILICLNMVTMMVETDDQSEYVTTILSRINLVFIVLFTGECVL
KLISLRHYYFTIGWNIFDFVVVILSIVGMFLAELIEKYFVSPTLFRVIRLARIGRILRLIKGAKGIRTLLFALMMS
LPALFNIGLLLFLVMFIYAIFGMSNFAYVKREVGIDDMFNFETFGNSMICLFQITTSAGWDGLLAPILNSKPPDCD
PNKVNPGSSVKGDCGNPSVGIFFFVSYIIISFLVVVNMYIAVILENFSVATEESAEPLSEDDFEMFYEVWEKFDPD
ATQFMEFEKLSQFAAALEPPLNLPQPNKLQLIAMDLPMVSGDRIHCLDILFAFTKRVLGESGEMDALRIQMEERFM
ASNPSKVSYQPITTTLKRKQEEVSAVIIQRAYRRHLLKRTVKQASFTYNKNKIKGGANLLIKEDMIIDRINENSIT
EKTDLTMSTAACPPSYDRVTKPIVEKHEQEGKDEKAKGK

Examples of gene therapy payloads useful for the treatment of Dravet syndrome include: nucleic acids (e.g., mRNA, cDNA, plasmid DNA, etc.), such as those encoding a functional galactosylceramidase or a functional fragment thereof; viral vectors (e.g., AAV, lentivirus, etc.); etc. In some embodiments, a gene therapy payload useful for the treatment of Dravet syndrome comprises a nucleic acid comprising a nucleobase sequence having at least 70% sequence identity (e.g., at least 70%, 75%, 80%, 85%, 90%, 95%, 96%, 97%, 98%, 99% or 100%) to the nucleobase sequence:

(SEQ ID NO: 809)
atggagcaaacagtgcttgtaccaccaggacctgacagcttcaacttcttcaccagagaatctcttgcgg
ctattgaaagacgcattgcagaagaaaaggcaaagaatcccaaaccagacaaaaaagatgacgacgaaaatggccc
aaagccaaatagtgacttggaagctggaaagaaccttccatttatttatggagacattcctccagagatggtgtca
gagcccctggaggacctggacccctactatatcaataagaaaacttttatagtattgaataaagggaaggccatct
tccggttcagtgccacctctgccctgtacattttaactcccttcaatcctcttaggaaaatagctattaagatttt
ggtacattcattattcagcatgctaattatgtgcactattttgacaaactgtgtgtttatgacaatgagtaaccct
cctgattggacaaagaatgtagaatacaccttcacaggaatatatacttttgaatcacttataaaaattattgcaa
ggggattctgtttagaagattttactttccttcgggatccatggaactggctcgatttcactgtcattacatttgc
gtacgtcacagagtttgtggacctgggcaatgtctcggcattgagaacattcagagttctccgagcattgaagacg
atttcagtcattccaggcctgaaaaccattgtgggagccctgatccagtctgtgaagaagctctcagatgtaatga
tcctgactgtgttctgtctgagcgtatttgctctaattgggctgcagctgttcatgggcaacctgaggaataaatg
tatacaatggcctcccaccaatgcttccttggaggaacatagtatagaaaagaatataactgtgaattataatggt
acacttataaatgaaactgtctttgagtttgactggaagtcatatattcaagattcaagatatcattatttcctgg
agggttttttagatgcactactatgtggaaatagctctgatgcaggccaatgtccagagggatatatgtgtgtgaa
agctggtagaaatcccaattatggctacacaagctttgataccttcagttgggcttttttgtccttgtttcgacta
atgactcaggacttctgggaaaatctttatcaactgacattacgtgctgctgggaaaacgtacatgatattttttg
tattggtcattttcttgggctcattctacctaataaatttgatcctggctgtggtggccatggcctacgaggaaca
gaatcaggccaccttggaagaagcagaacagaaagaggccgaatttcagcagatgattgaacagcttaaaaagcaa
caggaggcagctcagcaggcagcaacggcaactgcctcagaacattccagagagcccagtgcagcaggcaggctct
cagacagctcatctgaagcctctaagttgagttccaagagtgctaaggaaagaagaaatcggaggaagaaaagaaa
acagaaagagcagtctggtggggaagagaaagatgaggatgaattccaaaaatctgaatctgaggacagcatcagg
aggaaaggttttcgcttctccattgaagggaaccgattgacatatgaaaagaggtactcctccccacaccagtctt
tgttgagcatccgtggctccctattttcaccaaggcgaaatagcagaacaagccttttcagctttagagggcgagc
aaaggatgtgggatctgagaacgacttcgcagatgatgagcacagcacctttgaggataacgagagccgtagagat
tccttgtttgtgccccgacgacacggagagagacgcaacagcaacctgagtcagaccagtaggtcatcccggatgc
tggcagtgtttccagcgaatgggaagatgcacagcactgtggattgcaatggtgtggtttccttggttggtggacc
ttcagttcctacatcgcctgttggacagcttctgccagaggtgataatagataagccagctactgatgacaatgga
acaaccactgaaactgaaatgagaaagagaaggtcaagttctttccacgtttccatggactttctagaagatcctt
cccaaaggcaacgagcaatgagtatagccagcattctaacaaatacagtagaagaacttgaagaatccaggcagaa
atgcccaccctgttggtataaattttccaacatattcttaatctgggactgttctccatattggttaaaagtgaaa
catgttgtcaacctggttgtgatggacccatttgttgacctggccatcaccatctgtattgtcttaaatactcttt
tcatggccatggagcactatccaatgacggaccatttcaataatgtgcttacagtaggaaacttggttttcactgg
gatctttacagcagaaatgtttctgaaaattattgccatggatccttactattatttccaagaaggctggaatatc
tttgacggttttattgtgacgcttagcctggtagaacttggactcgccaatgtggaaggattatctgttctccgtt
catttcgattgctgcgagttttcaagttggcaaaatcttggccaacgttaaatatgctaataaagatcatcggcaa
ttccgtgggggctctgggaaatttaaccctcgtcttggccatcatcgtcttcatttttgccgtggtcggcatgcag
ctctttggtaaaagctacaaagattgtgtctgcaagatcgccagtgattgtcaactcccacgctggcacatgaatg
acttcttccactccttcctgattgtgttccgcgtgctgtgtggggagtggatagagaccatgtgggactgtatgga
ggttgctggtcaagccatgtgccttactgtcttcatgatggtcatggtgattggaaacctagtggtcctgaatctc
tttctggccttgcttctgagctcatttagtgcagacaaccttgcagccactgatgatgataatgaaatgaataatc
tccaaattgctgtggataggatgcacaaaggagtagcttatgtgaaaagaaaaatatatgaatttattcaacagtc
cttcattaggaaacaaaagattttagatgaaattaaaccacttgatgatctaaacaacaagaaagacagttgtatg
tccaatcatacagcagaaattgggaaagatcttgactatcttaaagatgtaaatggaactacaagtggtataggaa
ctggcagcagtgttgaaaaatacattattgatgaaagtgattacatgtcattcataaacaaccccagtcttactgt
gactgtaccaattgctgtaggagaatctgactttgaaaatttaaacacggaagactttagtagtgaatcggatctg
gaagaaagcaaagagaaactgaatgaaagcagtagctcatcagaaggtagcactgtggacatcggcgcacctgtag
aagaacagcccgtagtggaacctgaagaaactcttgaaccagaagcttgtttcactgaaggctgtgtacaaagatt
caagtgttgtcaaatcaatgtggaagaaggcagaggaaaacaatggtggaacctgagaaggacgtgtttccgaata
gttgaacataactggtttgagaccttcattgttttcatgattctccttagtagtggtgctctggcatttgaagata
tatatattgatcagcgaaagacgattaagacgatgttggaatatgctgacaaggttttcacttacattttcattct
ggaaatgcttctaaaatgggtggcatatggctatcaaacatatttcaccaatgcctggtgttggctggacttctta
attgttgatgtttcattggtcagtttaacagcaaatgccttgggttactcagaacttggagccatcaaatctctca
ggacactaagagctctgagacctctaagagccttatctcgatttgaagggatgagggtggttgtgaatgccctttt
aggagcaattccatccatcatgaatgtgcttctggtttgtcttatattctggctaattttcagcatcatgggcgta
aatttgtttgctggcaaattctaccactgtattaacaccacaactggtgacaggtttgacatcgaagacgtgaata
atcatactgattgcctaaaactaatagaaagaaatgagactgctcgatggaaaaatgtgaaagtaaactttgataa
tgtaggatttgggtatctctctttgcttcaagttgccacattcaaaggatggatggatataatgtatgcagcagtt
gattccagaaatgtggaactccagcctaagtatgaagaaagtctgtacatgtatctttactttgttattttcatca
tctttgggtccttcttcaccttgaacctgtttattggtgtcatcatagataatttcaaccagcagaaaaagaagtt
tggaggtcaagacatctttatgacagaagaacagaagaaatactataatgcaatgaaaaaattaggatcgaaaaaa
ccgcaaaagcctatacctcgaccaggaaacaaatttcaaggaatggtctttgacttcgtaaccagacaagtttttg
acataagcatcatgattctcatctgtcttaacatggtcacaatgatggtggaaacagatgaccagagtgaatatgt
gactaccattttgtcacgcatcaatctggtgttcattgtgctatttactggagagtgtgtactgaaactcatctct
ctacgccattattattttaccattggatggaatatttttgattttgtggttgtcattctctccattgtaggtatgt
ttcttgccgagctgatagaaaagtatttcgtgtcccctaccctgttccgagtgatccgtcttgctaggattggccg
aatcctacgtctgatcaaaggagcaaaggggatccgcacgctgctctttgctttgatgatgtcccttcctgcgttg
tttaacatcggcctcctactcttcctagtcatgttcatctacgccatctttgggatgtccaactttgcctatgtta
agagggaagttgggatcgatgacatgttcaactttgagacctttggcaacagcatgatctgcctattccaaattac
aacctctgctggctgggatggattgctagcacccattctcaacagtaagccacccgactgtgaccctaataaagtt
aaccctggaagctcagttaagggagactgtgggaacccatctgttggaattttcttttttgtcagttacatcatca
tatccttcctggttgtggtgaacatgtacatcgcggtcatcctggagaacttcagtgttgctactgaagaaagtgc
agagcctctgagtgaggatgactttgagatgttctatgaggtttgggagaagtttgatcccgatgcaactcagttc
atggaatttgaaaaattatctcagtttgcagctgcgcttgaaccgcctctcaatctgccacaaccaaacaaactcc
agctcattgccatggatttgcccatggtgagtggtgaccggatccactgtcttgatatcttatttgcttttacaaa
gcgggttctaggagagagtggagagatggatgctctacgaatacagatggaagagcgattcatggcttccaatcct
tccaaggtctcctatcagccaatcactactactttaaaacgaaaacaagaggaagtatctgctgtcattattcagc
gtgcttacagacgccaccttttaaagcgaactgtaaaacaagcttcctttacgtacaataaaaacaaaatcaaagg
tggggctaatcttcttataaaagaagacatgataattgacagaataaatgaaaactctattacagaaaaaactgat
ctgaccatgtccactgcagcttgtccaccttcctatgaccgggtgacaaagccaattgtggaaaaacatgagcaag
aaggcaaagatgaaaaagccaaagggaaataa.

### Molecular Payloads Targeting SCN1A

The SCN1A gene, and mutations therein, are implicated in Dravet syndrome, which predominantly affects neurons in the brain. Modulation of SCN1A expression and activity (e.g., by suppressing the expression of SCN1A or a mutant form thereof and/or activity of a wild-type or mutant form of a protein encoded by SCN1A; or by increasing expression of a functional sodium voltage-gated channel alpha subunit 1 or a functional fragment thereof; etc.) therefore in some embodiments can have a therapeutic effect in subjects with Dravet syndrome.

### Oligonucleotides

SCN1A expression and/or activity in some embodiments can be modulated by the use of oligonucleotides targeting SCN1A sequences.

In some embodiments, an oligonucleotide useful for the treatment of Dravet syndrome, e.g., targeting (e.g., directly or indirectly modulating the expression or activity of) SCN1A, comprises a region of complementarity to an SCN1A transcript provided in Table 4, e.g., provided by any one of SEQ ID NOs: 546-563.

In some embodiments, examples of oligonucleotides useful for the treatment of Dravet syndrome, e.g., targeting (e.g., directly or indirectly modulating the expression or activity of) SCN1A, are provided in International Patent Application Publication No. WO2021174036A1, published Feb. 26, 2021, entitled “Compounds and methods for modulating scn1a expression”; and U.S. Patent Application Publication No. US20180369275A1, published Dec. 27, 2018, entitled “Antisense oligomers for treatment of autosomal dominant mental retardation-5 and dravet syndrome”; the entire contents of each of which are herein incorporated by reference.

Certain oligonucleotides provided in this section may be useful in treating Dravet syndrome by modulating the activity of genes and/or gene products other than SCN1A genes/gene products, such as other genes/gene products associated with Dravet syndrome.

### Polypeptides

SCN1A expression and/or activity in some embodiments can be modulated by the use of SCN1A polypeptides or polypeptides that can interact with SCN1A (e.g., to modulate its activity). In some embodiments, a polypeptide useful in the treatment of epilepsy and/or pain disorders is a peptide, a protein, an enzyme, or an antibody that modulates SCN1A, e.g., by interacting with SCN1A or a protein encoded by SCN1A.

### Small Molecules

SCN1A expression and/or activity in some embodiments can be modulated by the use of small molecules that can modulate SCN1A (e.g., to modulate its activity, or its expression).

In some embodiments, examples of small molecules useful in the treatment of Dravet syndrome are provided in U.S. Patent Application Publication No. US20220073463A1, published Mar. 10, 2022, entitled “Formulations of t-type calcium channel modulators and methods of use thereof”; U.S. Patent Application Publication No. US20200306238A1, published Oct. 1, 2020, entitled “Treatment of CNS conditions”; U.S. Patent Application Publication No. US20190160078A1, published May 30, 2019, entitled “Ganaxolone for use in treating genetic epileptic disorders”; U.S. Patent Application Publication No. US20190091231A1, published Mar. 28, 2019, entitled “Methods of treating developmental disorders and/or seizure disorders with etifoxine”; U.S. Patent Application Publication No. US20200085838A1, published Mar. 19, 2020, entitled “Methods of treating epilepsy and neurodevelopmental disorders”; U.S. Patent Application Publication No. US20180296501A1, published Oct. 18, 2018, entitled “Methods of treating developmental encephalopathies”; and U.S. Patent Application Publication No. US20200000757A1, published Jan. 2, 2020, entitled “Methods of treating seizure disorders and prader-willi syndrome”; U.S. Patent Application Publication No. US20140309181A1, published Oct. 16, 2014, entitled “TREATMENT OF DISEASES RELATED TO ALPHA SUBUNITS OF SODIUM CHANNELS, VOLTAGE-GATED (SCNxA) WITH SMALL MOLECULES”; U.S. Patent Application Publication No. US20180333397A1, published Nov. 22, 2018, entitled “Methods for treating epilepsy”; U.S. Patent Application Publication No. US20140329908A1, published Nov. 6, 2014, entitled “Method For The Treatment of Dravet Syndrome”; and U.S. Patent Application Publication No. US20200377499A1, published Dec. 3, 2020, entitled “Ion channel modulators”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Dravet syndrome by modulating the activity of genes and gene products other than SCN1A genes/gene products, such as other genes/gene products associated with Dravet syndrome.

### Gene Therapies

SCN1A expression and/or activity in some embodiments can be modulated by the use of gene therapies that can modulate SCN1A (e.g., by delivery of nucleic acids encoding SCN1A or other molecules that interact with SCN1A).

In some embodiments, gene therapies, such as those involving administration of compounds encoding useful therapeutic agents, useful in the treatment of Dravet syndrome are provided in U.S. Patent Application Publication No. US20190127713A1, published May 2, 2019, entitled “Crispr/cas9-based repressors for silencing gene targets in vivo and methods of use”; U.S. Patent Application Publication No. US2021162072A1, published Jun. 3, 2021, entitled “Modified adeno-associated virus vectors and delivery thereof into the central nervous system”; and International Patent Application Publication No. WO2020210542A1, published Oct. 15, 2020, entitled “Long-lasting analgesia via targeted in vivo epigenetic repression”; the entire contents of each of which are herein incorporated by reference.

Certain gene therapies provided in this section may be useful in treating Dravet syndrome by modulating the activity of genes and gene products other than SCN1A genes/gene products, such as other genes/gene products associated with Dravet syndrome.

### Molecular Payloads for the Treatment of Batten Disease

Various molecular payloads may be useful in the treatment of Batten disease, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of Batten disease may include, in some embodiments, molecular payloads which modulate (e.g., increase or decrease) expression or activity of TPP1 and/or CLN3.

Examples of oligonucleotides useful for the treatment of Batten disease, e.g., oligonucleotides targeting (e.g., directly or indirectly modulating the expression or activity of) genes associated with Batten disease (e.g., TPP1, CLN3, etc.), include those listed in Table 17 below. In some embodiments, exon-skipping oligonucleotides targeting CLN3 are useful for the treatment of Batten disease (e.g., CLN3 Batten disease). Each oligonucleotide provided in Table 17 may have any modification pattern disclosed herein.

TABLE 17
Oligonucleotides for the treatment of Batten disease

| | | Oligo- | |
|---|---|---|---|
| SEQ | | nucleotide | |
| ID | Target | Sequence | |
| NO: | gene | (5′-3′) | Oligonucleotide Structure (5′-3′) |
| 248 | CLN3 | ACAACCCTCC | Aes Ces Aes Aes Ces Ces Ces Tes Ces Ces Ces Aes |
| | | CAACCACG | Aes Ces Ces Aes Ces Ge |
| 249 | CLN3 | GGGACAACCC | Ges Ges Ges Aes Ces Aes Aes Ces Ces Ces Tes Ces |
| | | TCCCAACC | Ces Ces Aes Aes Ces Ce |
| 250 | CLN3 | AGGGGACAAC | Aes Ges Ges Ges Ges Aes Ces Aes Aes Ces Ces Ces |
| | | CCTCCCAA | Tes Ces Ces Ces Aes Ae |
| 251 | CLN3 | CTTCCAGGGG | Ces Tes Tes Ces Ces Aes Ges Ges Ges Ges Aes Ces |
| | | ACAACCCT | Aes Aes Ces Ces Ces Te |
| 252 | CLN3 | CAGAGCTTCC | Ces Aes Ges Aes Ges Ces Tes Tes Ces Ces Aes Ges |
| | | AGGGGACA | Ges Ges Ges Aes Ces Ae |
| 253 | CLN3 | GACCGCAGAG | Ges Aes Ces Ces Ges Ces Aes Ges Aes Ges Ces Tes |
| | | CTTCCAGG | Tes Ces Ces Aes Ges Ge |
| 254 | CLN3 | AGTGAGACCG | Aes Ges Tes Ges Aes Ges Aes Ces Ces Ges Ces Aes |
| | | CAGAGCTT | Ges Aes Ges Ces Tes Te |
| 255 | CLN3 | AATAGAGTGA | Aes Aes Tes Aes Ges Aes Ges Tes Ges Aes Ges Aes |
| | | GACCGCAG | Ces Ces Ges Ces Aes Ge |
| 256 | CLN3 | AGGAGAATAG | Aes Ges Ges Aes Ges Aes Aes Tes Aes Ges Aes Ges |
| | | AGTGAGAC | Tes Ges Aes Ges Aes Ce |
| 257 | CLN3 | GGGACAGGAG | Ges Ges Ges Aes Ces Aes Ges Ges Aes Ges Aes Aes |
| | | AATAGAGT | Tes Aes Ges Aes Ges Te |
| 258 | CLN3 | AGCCTGGGAC | Aes Ges Ces Ces Tes Ges Ges Ges Aes Ces Aes Ges |
| | | AGGAGAAT | Ges Aes Ges Aes Aes Te |
| 259 | CLN3 | AGCACAGCCT | Aes Ges Ces Aes Ces Aes Ges Ces Ces Tes Ges Ges |
| | | GGGACAGG | Ges Aes Ces Aes Ges Ge |
| 260 | CLN3 | CCAGGAGCAC | Ces Ces Aes Ges Ges Aes Ges Ces Aes Ces Aes Ges |
| | | AGCCTGGG | Ces Ces Tes Ges Ges Ge |
| 261 | CLN3 | GTCCGCCAGG | Ges Tes Ces Ces Ges Ces Ces Aes Ges Ges Aes Ges |
| | | AGCACAGC | Ces Aes Ces Aes Ges Ce |
| 262 | CLN3 | AGGATGTCCG | Aes Ges Ges Aes Tes Ges Tes Ces Ces Ges Ces Ces |
| | | CCAGGAGC | Aes Ges Ges Aes Ges Ces |
| 263 | CLN3 | GGGAGGATGT | Ges Ges Ges Aes Ges Ges Aes Tes Ges Tes Ces Ces |
| | | CCGCCAGG | Ges Ces Ces Aes Ges Ge |
| 264 | CLN3 | TGGGGAGGAT | Tes Ges Ges Ges Ges Aes Ges Ges Aes Tes Ges Tes |
| | | GTCCGCCA | Ces Ces Ges Ces Ces Ae |
| 265 | CLN3 | GAGTGTGGGG | Ges Aes Ges Tes Ges Tes Ges Ges Ges Ges Aes Ges |
| | | AGGATGTC | Ges Aes Tes Ges Tes Ce |
| 266 | CLN3 | ATGACGAGTG | Aes Tes Ges Aes Ces Ges Aes Ges Tes Ges Tes Ges |
| | | TGGGGAGG | Ges Ges Ges Aes Ges Ge |
| 267 | CLN3 | ATTTGATGAC | Aes Tes Tes Tes Ges Aes Tes Ges Aes Ces Ges Aes |
| | | GAGTGTGG | Ges Tes Ges Tes Ges Ge |
| 268 | CLN3 | CAACAATTTG | Ces Aes Aes Ces Aes Aes Tes Tes Tes Ges Aes Tes |
| | | ATGACGAG | Ges Aes Ces Ges Aes Ge |
| 269 | CLN3 | GGAGCCAACA | Ges Ges Aes Ges Ces Ces Aes Aes Ces Aes Aes Tes |
| | | ATTTGATG | Tes Tes Ges Aes Tes Ge |
| 270 | CLN3 | CAAGAGGAGC | Ces Aes Aes Ges Aes Ges Ges Aes Ges Ces Ces Aes |
| | | CAACAATT | Aes Ces Aes Aes Tes Te |
| 271 | CLN3 | AAGGCCAAGA | Aes Aes Ges Ges Ces Ces Aes Aes Ges Aes Ges Ges |
| | | GGAGCCAA | Aes Ges Ces Ces Aes Ae |
| 272 | CLN3 | AGGTGAAGGC | Aes Ges Ges Tes Ges Aes Aes Ges Ges Ces Ces Aes |
| | | CAAGAGGA | Aes Ges Aes Ges Ges Ae |
| 273 | CLN3 | GCAGCAGGTG | Ges Ces Aes Ges Ces Aes Ges Ges Tes Ges Aes Aes |
| | | AAGGCCAA | Ges Ges Ces Ces Aes Ae |
| 274 | CLN3 | GTAGGGCAGC | Ges Tes Aes Ges Ges Ges Ces Aes Ges Ces Aes Ges |
| | | AGGTGAAG | Ges Tes Ges Aes Aes Ge |
| 275 | CLN3 | GACCTGTAGG | Ges Aes Ces Ces Tes Ges Tes Aes Ges Ges Ges Ces |
| | | GCAGCAGG | Aes Ges Ces Aes Ges Ge |
| 276 | CLN3 | ACCCAGACCT | Aes Ces Ces Ces Aes Ges Aes Ces Ces Tes Ges Tes |
| | | GTAGGGCA | Aes Ges Ges Ges Ces Ae |
| 277 | CLN3 | CCCTCACCCA | Ces Ces Ces Tes Ces Aes Ces Ces Ces Aes Ges Aes |
| | | GACCTGTA | Ces Ces Tes Ges Tes Ae |
| 278 | CLN3 | CACTACCCTC | Ces Aes Ces Tes Aes Ces Ces Ces Tes Ces Aes Ces |
| | | ACCCAGAC | Ces Ces Aes Ges Aes Ce |
| 279 | CLN3 | CCTCCCACTA | Ces Ces Tes Ces Ces Ces Aes Ces Tes Aes Ces Ces |
| | | CCCTCACC | Ces Tes Ces Aes Ces Ce |
| 280 | CLN3 | CCCTGCCTCC | Ces Ces Ces Tes Ges Ces Ces Tes Ces Ces Ces Aes |
| | | CACTACCC | Ces Tes Aes Ces Ces Ce |
| 281 | CLN3 | GCCCACCCTG | Tes Ces Ces Ces Aes Ces Ces Ces Tes Ges Ces Ces |
| | | CCTCCCAC | Ges Ces Ces Ces Aes Ce |
| 282 | CLN3 | CTCCTGCCCA | Ces Tes Ces Ces Tes Ges Ces Ces Ces Aes Ces Ces |
| | | CCCTGCCT | Ces Tes Ges Ces Ces Te |
| 283 | CLN3 | CTCAGCTCCT | Ces Ces Ces Aes Ges Ces Tes Ces Ces Tes Ges Ces |
| | | GCCCACCC | Ces Tes Aes Ces Ces Ce |
| 284 | CLN3 | CCTTTCTCAG | Ces Ces Tes Tes Tes Ces Tes Ces Aes Ges Ces Tes |
| | | CTCCTGCC | Ces Ces Tes Ges Ces Ce |
| 285 | CLN3 | CCTCCCCTTT | Ces Aes Tes Ces Tes Ces Ces Tes Tes Tes Ces Tes |
| | | CTCAGCTC | Ces Ces Ges Ces Ces Ce |
| 286 | CLN3 | CCCAGCCTCC | Tes Ces Ces Aes Ges Ces Ces Tes Ces Ces Ces Ces |
| | | CCTTTCTC | Ces Tes Tes Ces Tes Ce |
| 287 | CLN3 | GCCATCCCAG | Tes Ces Ces Aes Tes Ces Ces Ces Aes Ges Ces Ces |
| | | CCTCCCCT | Ges Ces Ces Ces Ces Te |

Abbreviations used in Table 17:
s = phosphorothioate internucleoside linkage; e = 2′-MOE modified sugar. In each sequence listed in Table 17, each T may be optionally and independently replaced with a U, and each U may be optionally and independently replaced with a T.

Examples of small molecules useful for the treatment of Batten disease include: tamoxifen, raloxifene, toremifene, clomifene, ospemifene, bazedoxifene, nafoxidine, lasofoxifene, zuclomiphene, afimoxifene, N-desmethyltamoxifen, droloxifene, tamoxifen aziridine, idoxifene, 2-methyl-4-hydroxytamoxifen, endoxifen, phenyltoloxamine, tamoxifen N-oxide, tamoxifen epoxide, diethylaminoethoxyhexestrol, etoloxamine, tesmilifene, tamoxifen-d5, myoparkil,

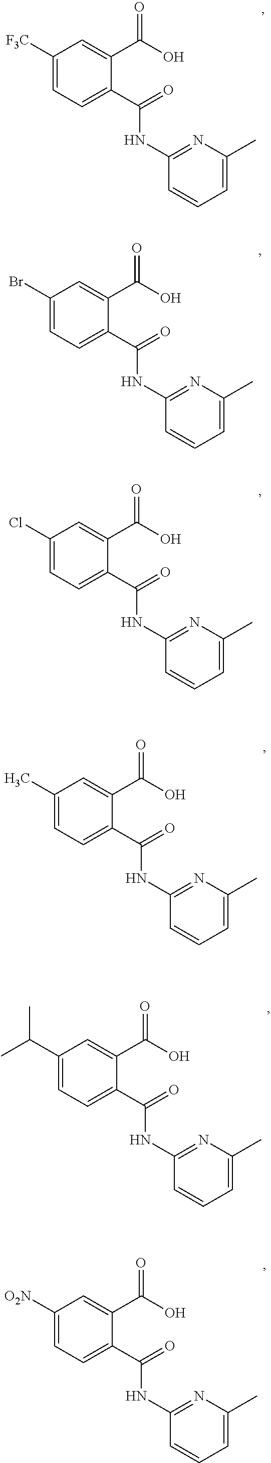

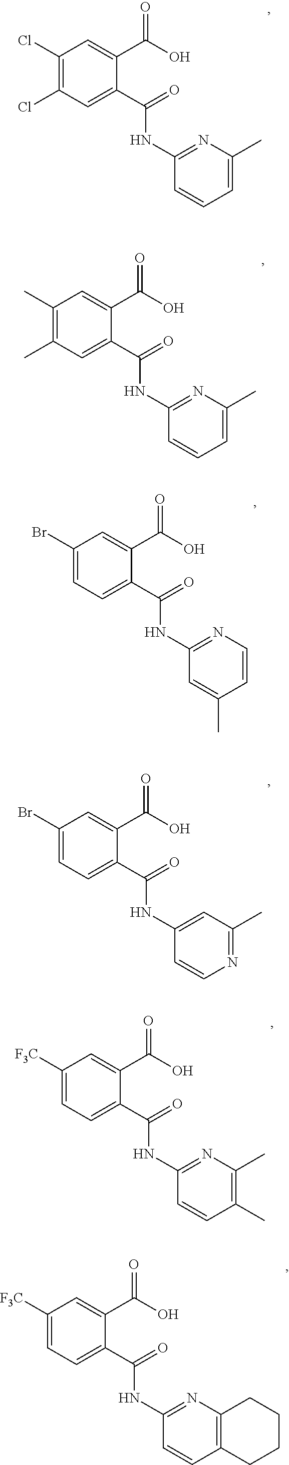

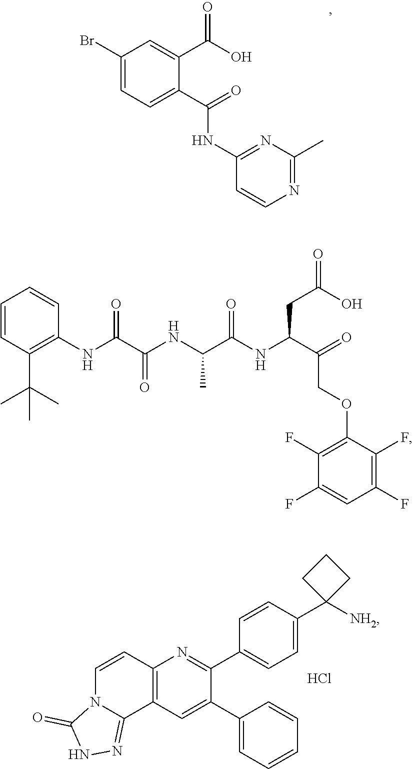 [SMILES: Cc1nccc(NC(=O)c2ccc(Br)cc2C(=O)O)n1 ; C[C@H](NC(=O)C(=O)Nc1ccccc1C(C)(C)C)C(=O)N[C@@H](CC(=O)O)C(=O)COc1c(F)c(F)cc(F)c1F ; NC1(c2ccc(-c3nc4ccn5c(=O)[nH]nc5c4cc3-c3ccccc3)cc2)CCC1 ; Cl]

and pharmaceutically acceptable salts, co-crystals, tautomers, stereoisomers, solvates, hydrates, polymorphs, isotopically enriched compounds, and prodrugs thereof.

Examples of polypeptides useful for the treatment of other neurological disorders include antibodies, proteins, peptides, and enzymes.

### Molecular Payloads Targeting TPP1

The TPP1 gene, and mutations therein, are implicated in Batten disease, which affects cells of the nervous system, including neurons of the CNS. Modulation of TPP1 expression and activity (e.g., by suppressing the expression of TPP1 or a mutant form thereof and/or activity of a wild-type or mutant form of a protein encoded by TPP1, or by increasing expression of a functional form of tripeptidyl peptidase 1 or a functional fragment thereof) therefore in some embodiments can have a therapeutic effect in subjects with a disorder described herein.

### Oligonucleotides

TPP1 expression and/or activity in some embodiments can be modulated by the use of oligonucleotides targeting TPP1 sequences.

In some embodiments, an oligonucleotide useful for the treatment of Batten disease, e.g., targeting (e.g., directly or indirectly modulating the expression or activity of) TPP1, comprises a region of complementarity to a TPP1 transcript, such as a TPP1 transcript provided in Table 4, e.g., provided by NM_000391.4 (SEQ ID NO: 846).

### Polypeptides

TPP1 expression and/or activity in some embodiments can be modulated by the use of TPP1 polypeptides or polypeptides that can interact with TPP1 (e.g., to modulate its activity). In some embodiments, a polypeptide useful in the treatment of Batten disease is a peptide, a protein, an enzyme, or an antibody that modulates TPP1, e.g., by interacting with TPP1 or a protein encoded by TPP1. In some embodiments, a polypeptide useful in the treatment of Batten disease is a peptide, a protein, an enzyme, or an antibody that modulates TPP1, e.g., by interacting with TPP1 or a protein encoded by TPP1, or by providing a protein encoded by TPP1 or a functional fragment thereof (e.g., as an enzyme replacement therapy).

In some embodiments, polypeptides useful in the treatment of Batten disease are provided in U.S. Ser. No. 10/279,015B2, published May 7, 2019, entitled “TPP-1 formulations and methods for treating CLN2 disease”; and US20120308544A1, published Dec. 6, 2012, entitled “Substances and Methods for the Treatment of Lysosmal Storage Diseases”; the entire contents of each of which are herein incorporated by reference.

### Small Molecules

TPP1 expression and/or activity in some embodiments can be modulated by the use of small molecules that can modulate TPP1 (e.g., to modulate its activity, or its expression).

In some embodiments, examples of small molecules useful in the treatment of Batten disease are provided in WO2023012353, published Feb. 9, 2023, entitled “Compounds for use in the therapeutic treatment of batten disease”; WO2022212268A1, published Mar. 28, 2022, entitled “Methods and compositions for treating lysosomal storage disorders”; WO2022023573A2, published Feb. 3, 2022, entitled “Products for treating the jncl disease”; WO2022115612A1, published Jun. 2, 2022, entitled “Inhibition of caspase pathway as a treatment for lysosomal storage disorders”; US20170304339A1, published Oct. 26, 2017, entitled “Compositions and methods for the treatment of lysosomal storage disorders and disorders characterized by lysosomal dysfunction”; US20210230160A1, published Jul. 29, 2021, entitled “Phosphodiesterase inhibitors”; and U.S. Pat. No. 6,821,995B1, published Nov. 23, 2004, entitled “Method of treating batten disease”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

### Gene Therapies

TPP1 expression and/or activity in some embodiments can be modulated by the use of gene therapies that can modulate TPP1 (e.g., by delivery of nucleic acids encoding TPP1 or other molecules that interact with TPP1). In some embodiments, a gene therapy payload useful in the treatment of glycogen synthesis disorders is a payload that modulates TPP1, e.g., by interacting with TPP1 or a protein encoded by TPP1, by stimulating expression of TPP1 (such as by providing a molecule that encodes TPP1), or by suppressing expression of TPP1 (such as by providing a molecule that encodes a suppressor of TPP1, such as a mutant form of TPP1).

In some embodiments, gene therapies, such as those involving administration of a compounds encoding useful therapeutic agents, useful in the treatment of Batten disease are provided in U.S. Pat. No. 9,849,195B2, published Dec. 26, 2017, entitled “Methods and compositions for treating brain diseases”; US20200360491A1, published Nov. 19, 2020, entitled “Treatment of lysosomal storage disease in the eye through administration of aavs expressing tpp1”; U.S. Ser. No. 11/591,614B2, published Feb. 28, 2023, entitled “Gene therapy for ceroid lipofuscinoses”; and U.S. Ser. No. 10/279,015B2, published May 7, 2019, entitled “TPP-1 formulations and methods for treating CLN2 disease”; the entire contents of each of which are herein incorporated by reference.

Certain gene therapies provided in this section may be useful in treating Batten disease by modulating the activity of genes and gene products other than TPP1 genes/gene products, such as other genes/gene products associated with Batten disease.

### Molecular Payloads Targeting CLN3

The CLN3 gene, and mutations therein, are implicated in Batten disease, which affects cells of the nervous system, including neurons of the CNS. Modulation of CLN3 expression and activity (e.g., by suppressing the expression of CLN3 or a mutant form thereof and/or activity of a wild-type or mutant form of a protein encoded by CLN3) therefore in some embodiments can have a therapeutic effect in subjects with Batten disease.

### Oligonucleotides

CLN3 expression and/or activity in some embodiments can be modulated by the use of oligonucleotides targeting CLN3 sequences.

In some embodiments, an oligonucleotide useful for the treatment of Batten disease, e.g., targeting (e.g., directly or indirectly modulating the expression or activity of) CLN3, comprises a region of complementarity to a CLN3 transcript provided in Table 4, e.g., provided by any one of SEQ ID NOs: 581-586.

In some embodiments, examples of oligonucleotides useful for the treatment of Batten disease, e.g., targeting (e.g., directly or indirectly modulating the expression or activity of, such as by modulating splicing of a CLN3 transcript) CLN3, are provided in International Patent Application Publication No. WO2020055917A1, published Mar. 19, 2020, entitled “Compounds and methods for modulating cln3 expression”; and WO2022150369A1, published Jul. 14, 2022, entitled “Compounds for the treatment of batten disease”; the entire contents of each of which are herein incorporated by reference.

Certain oligonucleotides provided in this section may be useful in treating Batten disease by modulating the activity of genes and/or gene products other than CLN3 genes/gene products, such as other genes/gene products associated with Batten disease.

### Polypeptides

CLN3 expression and/or activity in some embodiments can be modulated by the use of CLN3 polypeptides or polypeptides that can interact with CLN3 (e.g., to modulate its activity).

In some embodiments, examples of polypeptides (e.g., peptides; proteins, such as enzymes; antibodies; etc.) useful in the treatment of Batten disease are provided in Nelson T, et al. “Lack of specificity of antibodies raised against CLN3, the lysosomal/endosomal transmembrane protein mutated in juvenile Batten disease.” Biosci Rep. (2017) 37(6):BSR20171229; the entire contents of which are herein incorporated by reference.

Certain polypeptides provided in this section may be useful in treating Batten disease by modulating the activity of genes and gene products other than CLN3 genes/gene products, such as other genes/gene products associated with Batten disease.

### Small Molecules

CLN3 expression and/or activity in some embodiments can be modulated by the use of small molecules that can modulate CLN3 (e.g., to modulate its activity, or its expression).

In some embodiments, examples of small molecules useful in the treatment of Batten disease are provided in U.S. Patent Application Publication No. US20190160078A1, published May 30, 2019, entitled “Ganaxolone for use in treating genetic epileptic disorders”; WO2023012353, published Feb. 9, 2023, entitled “Compounds for use in the therapeutic treatment of batten disease”; WO2022212268A1, published Mar. 28, 2022, entitled “Methods and compositions for treating lysosomal storage disorders”; WO2022023573A2, published Feb. 3, 2022, entitled “Products for treating the jncl disease”; WO2022115612A1, published Jun. 2, 2022, entitled “Inhibition of caspase pathway as a treatment for lysosomal storage disorders”; US20170304339A1, published Oct. 26, 2017, entitled “Compositions and methods for the treatment of lysosomal storage disorders and disorders characterized by lysosomal dysfunction”; US20210230160A1, published Jul. 29, 2021, entitled “Phosphodiesterase inhibitors”; and U.S. Pat. No. 6,821,995B1, published Nov. 23, 2004, entitled “Method of treating batten disease”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Batten disease by modulating the activity of genes and gene products other than CLN3 genes/gene products, such as other genes/gene products associated with Batten disease.

### Gene Therapies

CLN3 expression and/or activity in some embodiments can be modulated by the use of gene therapies that can modulate CLN3 (e.g., by delivery of nucleic acids encoding CLN3 or other molecules that interact with CLN3).

In some embodiments, gene therapies, such as those involving administration of a compounds encoding useful therapeutic agents, useful in the treatment of Batten disease are provided in U.S. Patent Application Publication No. US2021162072A1, published Jun. 3, 2021, entitled “Modified adeno-associated virus vectors and delivery thereof into the central nervous system”; and WO1997008308A1, published Mar. 6, 1997, entitled “Batten disease gene”; the entire contents of each of which are herein incorporated by reference.

Certain gene therapies provided in this section may be useful in treating Batten disease by modulating the activity of genes and gene products other than CLN3 genes/gene products, such as other genes/gene products associated with Batten disease.

### Molecular Payloads for the Treatment of Other Neurological Disorders

Various molecular payloads may be useful in the treatment of other neurological disorders, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of epilepsy may include, in some embodiments, molecular payloads which modulate (e.g., increase or decrease) expression or activity of GYS1, PrP, GFAP, LSD, SCN9A, UBE3A, and/or VLA-4.

Examples of oligonucleotides useful for the treatment of other neurological disorders, e.g., oligonucleotides targeting (e.g., directly or indirectly modulating the expression or activity of) genes associated with neurological disorders (e.g., GYS1, PrP, GFAP, LSD, SCN9A, UBE3A, VLA-4, etc.), include those listed in Table 18 below. Each oligonucleotide provided in Table 18 may have any modification pattern disclosed herein.

TABLE 18
Oligonucleotides for the treatment of other neurological disorders

| SEQ | Target | Oligonucleotide | |
|---|---|---|---|
| ID NO: | gene | Sequence (5′-3′) | Oligonucleotide Structure (5′-3′) |
| 301 | GYS1 | CATGCTTCAT | CATGCTTCATTTCTTTATTG, as a gapmer, PMO, or unmodified |
| | | TTCTTTATTG | oligodeoxyribonucleotide or oligoribonucleotide |
| 302 | GYS1 | CCGACTCAGG | CCGACTCAGGTAGGGTGAGC, with modification pattern eeeee- |
| | | TAGGGTGAGC | dddddddddd-eeeee, with internucleoside linkages soooo- |
| | | | ssssssssss-ooss, and each C is a 5-methyl C |
| 303 | GYS1 | CTTGGTGACC | CTTGGTGACCGGTAGAGTTA, with modification pattern eeeee- |
| | | GGTAGAGTTA | dddddddddd-eeeee, with internucleoside linkages soooo- |
| | | | ssssssssss-ooss, and each C is a 5-methyl C |
| 304 | GYS1 | GAGAGGCATG | GAGAGGCATGGCTACTGCGG, with modification pattern eeeee- |
| | | GCTACTGCGG | dddddddddd-eeeee, with internucleoside linkages soooo- |
| | | | ssssssssss-ooss, and each C is a 5-methyl C |
| 305 | GYS1 | CGGCTGAGAG | CGGCTGAGAGGCATGGCTAC, with modification pattern eeeee- |
| | | GCATGGCTAC | dddddddddd-eeeee, with internucleoside linkages soooo- |
| | | | ssssssssss-ooss, and each C is a 5-methyl C |
| 306 | GYS1 | TCTTCCAATC | TCTTCCAATCCTGGAAGCGA, with modification pattern eeeee- |
| | | CTGGAAGCGA | dddddddddd-eeeee, with internucleoside linkages soooo- |
| | | | ssssssssss-ooss, and each C is a 5-methyl C |
| 307 | GYS1 | ATGGTCCCAC | ATGGTCCCACCAGATAGTAG, with modification pattern eeeee- |
| | | CAGATAGTAG | dddddddddd-eeeee, with internucleoside linkages soooo- |
| | | | ssssssssss-ooss, and each C is a 5-methyl C |
| 308 | GYS1 | CGTGTATGGT | CGTGTATGGTCCCACCAGAT, with modification pattern eeeee- |
| | | CCCACCAGAT | dddddddddd-eeeee, with internucleoside linkages soooo- |
| | | | ssssssssss-ooss, and each C is a 5-methyl C |
| 309 | GYS1 | AGGTGTTGAG | AGGTGTTGAGCCTCGATTGC, with modification pattern eeeee- |
| | | CCTCGATTGC | dddddddddd-eeeee, with internucleoside linkages soooo- |
| | | | ssssssssss-ooss, and each C is a 5-methyl C |
| 310 | GYS1 | TGACTGTATT | TGACTGTATTGGCTGTGTCC, with modification pattern eeeee- |
| | | GGCTGTGTCC | dddddddddd-eeeee, with internucleoside linkages soooo- |
| | | | ssssssssss-ooss, and each C is a 5-methyl C |
| 311 | GYS1 | CTCCTTGACT | CTCCTTGACTGTATTGGCTG, with modification pattern eeeee- |
| | | GTATTGGCTG | dddddddddd-eeeee, with internucleoside linkages soooo- |
| | | | ssssssssss-ooss, and each C is a 5-methyl C |
| 312 | GYS1 | GTAGAGCTTC | GTAGAGCTTCCTCCCAAATT, with modification pattern eeeee- |
| | | CTCCCAAATT | dddddddddd-eeeee, with internucleoside linkages soooo- |
| | | | ssssssssss-ooss, and each C is a 5-methyl C |
| 313 | GYS1 | CCTCCGATCC | CCTCCGATCCAGAATGTAAA, with modification pattern eeeee- |
| | | AGAATGTAAA | dddddddddd-eeeee, with internucleoside linkages soooo- |
| | | | ssssssssss-ooss, and each C is a 5-methyl C |
| 314 | GYS1 | ACTTCCAATC | ACTTCCAATCTAGCAAGTCC, with modification pattern eeeee- |
| | | TAGCAAGTCC | dddddddddd-eeeee, with internucleoside linkages soooo- |
| | | | ssssssssss-ooss, and each C is a 5-methyl C |
| 315 | GYS1 | TCCCGTGGCT | TCCCGTGGCTCTTCCTCATC, with modification pattern eeeee- |
| | | CTTCCTCATC | dddddddddd-eeeee, with internucleoside linkages soooo- |
| | | | ssssssssss-ooss, and each C is a 5-methyl C |
| 316 | GYS1 | TGTGGAGGAG | TGTGGAGGAGGAACAGGAGG, with modification pattern eeeee- |
| | | GAACAGGAGG | dddddddddd-eeeee, with internucleoside linkages soooo- |
| | | | ssssssssss-ooss, and each C is a 5-methyl C |
| 317 | GYS1 | TGCCACCTGT | TGCCACCTGTGGAGGAGGAA, with modification pattern eeeee- |
| | | GGAGGAGGAA | dddddddddd-eeeee, with internucleoside linkages soooo- |
| | | | ssssssssss-ooss, and each C is a 5-methyl C |
| 318 | GYS1 | CTGGAGGGCC | CTGGAGGGCCCAGTGTCCAC, with modification pattern eeeee- |
| | | CAGTGTCCAC | dddddddddd-eeeee, with internucleoside linkages soooo- |
| | | | ssssssssss-ooss, and each C is a 5-methyl C |
| 319 | GYS1 | GTGAGCTGGA | GTGAGCTGGAGGGCCCAGTG, with modification pattern eeeee- |
| | | GGGCCCAGTG | dddddddddd-eeeee, with internucleoside linkages soooo- |
| | | | ssssssssss-ooss, and each C is a 5-methyl C |
| 320 | GYS1 | CATAGGCCCT | CATAGGCCCTCTGCGAGAGG, with modification pattern eeeee- |
| | | CTGCGAGAGG | dddddddddd-eeeee, with internucleoside linkages soooo- |
| | | | ssssssssss-ooss, and each C is a 5-methyl C |
| 321 | GYS1 | ATCTGCATAG | ATCTGCATAGGCCCTCTGCG, with modification pattern eeeee- |
| | | GCCCTCTGCG | dddddddddd-eeeee, with internucleoside linkages soooo- |
| | | | ssssssssss-ooss, and each C is a 5-methyl C |
| 322 | GYS1 | TTCAGGCACC | TTCAGGCACCCTCCCATCTG, with modification pattern eeeee- |
| | | CTCCCATCTG | dddddddddd-eeeee, with internucleoside linkages soooo- |
| | | | ssssssssss-ooss, and each C is a 5-methyl C |
| 323 | GYS1 | ACTCAAGAGT | ACTCAAGAGTCTGGAGTGGG, with modification pattern eeeee- |
| | | CTGGAGTGGG | dddddddddd-eeeee, with internucleoside linkages soooo- |
| | | | ssssssssss-ooss, and each C is a 5-methyl C |
| 324 | GYS1 | GGCTGGAGTG | GGCTGGAGTGTCTGAAACAG, with modification pattern eeeee- |
| | | TCTGAAACAG | dddddddddd-eeeee, with internucleoside linkages soooo- |
| | | | ssssssssss-ooss, and each C is a 5-methyl C |
| 325 | GYS1 | TGGAGCTCAA | TGGAGCTCAAGGGCTGGAGT, with modification pattern eeeee- |
| | | GGGCTGGAGT | dddddddddd-eeeee, with internucleoside linkages soooo- |
| | | | ssssssssss-ooss, and each C is a 5-methyl C |
| 326 | GYS1 | CCAAGAAAGG | CCAAGAAAGGCACGGCGGCG, with modification pattern eeeee- |
| | | CACGGCGGCG | dddddddddd-eeeee, with internucleoside linkages soooo- |
| | | | ssssssssss-ooss, and each C is a 5-methyl C |
| 327 | GYS1 | CTGGAGACTC | CTGGAGACTCCAGATCAGTG, with modification pattern eeeee- |
| | | CAGATCAGTG | dddddddddd-eeeee, with internucleoside linkages soooo- |
| | | | ssssssssss-ooss, and each C is a 5-methyl C |
| 328 | GYS1 | AAACAATGGC | AAACAATGGCAGATGCCTGG, with modification pattern eeeee- |
| | | AGATGCCTGG | dddddddddd-eeeee, with internucleoside linkages soooo- |
| | | | ssssssssss-ooss, and each C is a 5-methyl C |
| 329 | GYS1 | CCTAAAACCT | CCTAAAACCTCTGGCATTGA, with modification pattern eeeee- |
| | | CTGGCATTGA | dddddddddd-eeeee, with internucleoside linkages soooo- |
| | | | ssssssssss-ooss, and each C is a 5-methyl C |
| 330 | GYS1 | CCTGGAAGCC | CCTGGAAGCCAATAAACCAG, with modification pattern eeeee- |
| | | AATAAACCAG | dddddddddd-eeeee, with internucleoside linkages soooo- |
| | | | ssssssssss-ooss, and each C is a 5-methyl C |
| 331 | GYS1 | GCCACAGCCT | GCCACAGCCTGGAAGCCAAT, with modification pattern eeeee- |
| | | GGAAGCCAAT | dddddddddd-eeeee, with internucleoside linkages soooo- |
| | | | ssssssssss-ooss, and each C is a 5-methyl C |
| 332 | GYS1 | GCCACACAGA | GCCACACAGAATCCAACATG, with modification pattern eeeee- |
| | | ATCCAACATG | dddddddddd-eeeee, with internucleoside linkages soooo- |
| | | | ssssssssss-ooss, and each C is a 5-methyl C |
| 333 | GYS1 | TCGGGAAGCC | TCGGGAAGCCACACAGAATC, with modification pattern eeeee- |
| | | ACACAGAATC | dddddddddd-eeeee, with internucleoside linkages soooo- |
| | | | ssssssssss-ooss, and each C is a 5-methyl C |
| 334 | GYS1 | CCTGAAATGT | CCTGAAATGTCCTAACTCTG, with modification pattern eeeee- |
| | | CCTAACTCTG | dddddddddd-eeeee, with internucleoside linkages soooo- |
| | | | ssssssssss-ooss, and each C is a 5-methyl C |
| 335 | GYS1 | TTAATCCCTG | TTAATCCCTGAAATGTCCTA, with modification pattern eeeee- |
| | | AAATGTCCTA | dddddddddd-eeeee, with internucleoside linkages soooo- |
| | | | ssssssssss-ooss, and each C is a 5-methyl C |
| 336 | GYS1 | AATCTGTCGA | AATCTGTCGACAGAGCTACT, with modification pattern eeeee- |
| | | CAGAGCTACT | dddddddddd-eeeee, with internucleoside linkages soooo- |
| | | | ssssssssss-ooss, and each C is a 5-methyl C |
| 337 | GYS1 | GACAAGCAAT | GACAAGCAATCTGTCGACAG, with modification pattern eeeee- |
| | | CTGTCGACAG | dddddddddd-eeeee, with internucleoside linkages soooo- |
| | | | ssssssssss-ooss, and each C is a 5-methyl C |
| 338 | GYS1 | TGTGTATCAC | TGTGTATCACCGCACCAGGT, with modification pattern eeeee- |
| | | CGCACCAGGT | dddddddddd-eeeee, with internucleoside linkages soooo- |
| | | | ssssssssss-ooss, and each C is a 5-methyl C |
| 339 | GYS1 | GAAATGGAGG | GAAATGGAGGACCGTGAGCA, with modification pattern eeeee- |
| | | ACCGTGAGCA | dddddddddd-eeeee, with internucleoside linkages soooo- |
| | | | ssssssssss-ooss, and each C is a 5-methyl C |
| 340 | GYS1 | TGCTCCTTTG | TGCTCCTTTGAAGAACACAC, with modification pattern eeeee- |
| | | AAGAACACAC | dddddddddd-eeeee, with internucleoside linkages soooo- |
| | | | ssssssssss-ooss, and each C is a 5-methyl C |
| 341 | GYS1 | GCAGAAAGGT | GCAGAAAGGTGTCTGGTCCA, with modification pattern eeeee- |
| | | GTCTGGTCCA | dddddddddd-eeeee, with internucleoside linkages soooo- |
| | | | ssssssssss-ooss, and each C is a 5-methyl C |
| 342 | GYS1 | AAGGCAGAAA | AAGGCAGAAAGGTGTCTGGT, with modification pattern eeeee- |
| | | GGTGTCTGGT | dddddddddd-eeeee, with internucleoside linkages soooo- |
| | | | ssssssssss-ooss, and each C is a 5-methyl C |
| 343 | GYS1 | TGACAGACAT | TGACAGACATTCTGCCCTCA, with modification pattern eeeee- |
| | | TCTGCCCTCA | dddddddddd-eeeee, with internucleoside linkages soooo- |
| | | | ssssssssss-ooss, and each C is a 5-methyl C |
| 344 | GYS1 | AGTGGGCTGA | AGTGGGCTGAGCACTTGTGG, with modification pattern eeeee- |
| | | GCACTTGTGG | dddddddddd-eeeee, with internucleoside linkages soooo- |
| | | | ssssssssss-ooss, and each C is a 5-methyl C |
| 345 | GYS1 | AGCCACTGGG | AGCCACTGGGACCCAGAACC, with modification pattern eeeee- |
| | | ACCCAGAACC | dddddddddd-eeeee, with internucleoside linkages soooo- |
| | | | ssssssssss-ooss, and each C is a 5-methyl C |
| 346 | GYS1 | TTCAAGAAGC | TTCAAGAAGCCGGTGGGCTC, with modification pattern eeeee- |
| | | CGGTGGGCTC | dddddddddd-eeeee, with internucleoside linkages soooo- |
| | | | ssssssssss-ooss, and each C is a 5-methyl C |
| 347 | GYS1 | GCAGAAAGGC | GCAGAAAGGCCTCGAGGTAC, with modification pattern eeeee- |
| | | CTCGAGGTAC | dddddddddd-eeeee, with internucleoside linkages soooo- |
| | | | ssssssssss-ooss, and each C is a 5-methyl C |
| 348 | GYS1 | CCCCCCAGGG | CCCCCCAGGGCCTAGGACGC, with modification pattern eeeee- |
| | | CCTAGGACGC | dddddddddd-eeeee, with internucleoside linkages soooo- |
| | | | ssssssssss-ooss, and each C is a 5-methyl C |
| 349 | GYS1 | ACAGCATTGA | ACAGCATTGAGTCTGCCATC, with modification pattern eeeee- |
| | | GTCTGCCATC | dddddddddd-eeeee, with internucleoside linkages soooo- |
| | | | ssssssssss-ooss, and each C is a 5-methyl C |
| 350 | GYS1 | TGGCCTGACT | TGGCCTGACTGGATGCTGGA, with modification pattern eeeee- |
| | | GGATGCTGGA | dddddddddd-eeeee, with internucleoside linkages soooo- |
| | | | ssssssssss-ooss, and each C is a 5-methyl C |
| 351 | GYS1 | ATTGATCTAA | ATTGATCTAACTCTGTCCCA, with modification pattern eeeee- |
| | | CTCTGTCCCA | dddddddddd-eeeee, with internucleoside linkages soooo- |
| | | | ssssssssss-ooss, and each C is a 5-methyl C |
| 352 | GYS1 | ATCCTTGGAT | ATCCTTGGATTAAAAGAGTG, with modification pattern eeeee- |
| | | TAAAAGAGTG | dddddddddd-eeeee, with internucleoside linkages soooo- |
| | | | ssssssssss-ooss, and each C is a 5-methyl C |
| 353 | GYS1 | GACCAAAACT | GACCAAAACTCCCAGATTTC, with modification pattern eeeee- |
| | | CCCAGATTTC | dddddddddd-eeeee, with internucleoside linkages soooo- |
| | | | ssssssssss-ooss, and each C is a 5-methyl C |
| 354 | GYS1 | AGCCACATGT | AGCCACATGTAGGGACCACA, with modification pattern eeeee- |
| | | AGGGACCACA | dddddddddd-eeeee, with internucleoside linkages soooo- |
| | | | ssssssssss-ooss, and each C is a 5-methyl C |
| 355 | GYS1 | CATGCTTCAT | CATGCTTCATTTCTTTATTG, with modification pattern eeeee- |
| | | TTCTTTATTG | dddddddddd-eeeee, with internucleoside linkages soooo- |
| | | | ssssssssss-ooss, and each C is a 5-methyl C |
| 356 | GYS1 | GGCCCATGCT | GGCCCATGCTTCATTTCTTT, with modification pattern eeeee- |
| | | TCATTTCTTT | dddddddddd-eeeee, with internucleoside linkages soooo- |
| | | | ssssssssss-ooss, and each C is a 5-methyl C |
| 357 | GYS1 | TCAGAGATAG | TCAGAGATAGCCAGAGAGAG, with modification pattern eeeee- |
| | | CCAGAGAGAG | dddddddddd-eeeee, with internucleoside linkages soooo- |
| | | | ssssssssss-ooss, and each C is a 5-methyl C |
| 358 | GYS1 | CCCTACTGTC | CCCTACTGTCTCATGACTTA, with modification pattern eeeee- |
| | | TCATGACTTA | dddddddddd-eeeee, with internucleoside linkages soooo- |
| | | | ssssssssss-ooss, and each C is a 5-methyl C |
| 359 | GYS1 | GAGGCCTCAG | GAGGCCTCAGCAAATGCCAG, with modification pattern eeeee- |
| | | CAAATGCCAG | dddddddddd-eeeee, with internucleoside linkages soooo- |
| | | | ssssssssss-ooss, and each C is a 5-methyl C |
| 360 | GYS1 | CCTCCAGCAA | CCTCCAGCAATGTATTTTAA, with modification pattern eeeee- |
| | | TGTATTTTAA | dddddddddd-eeeee, with internucleoside linkages soooo- |
| | | | ssssssssss-ooss, and each C is a 5-methyl C |
| 361 | GYS1 | AGGAATCAGA | AGGAATCAGAGGGTTCTGTG, with modification pattern eeeee- |
| | | GGGTTCTGTG | dddddddddd-eeeee, with internucleoside linkages soooo- |
| | | | ssssssssss-ooss, and each C is a 5-methyl C |
| 362 | GYS1 | AGCCCTCTCT | AGCCCTCTCTTTTATGACAA, with modification pattern eeeee- |
| | | TTTATGACAA | dddddddddd-eeeee, with internucleoside linkages soooo- |
| | | | ssssssssss-ooss, and each C is a 5-methyl C |
| 363 | GYS1 | ACAAGCTAAA | ACAAGCTAAAGACTTAAACT, with modification pattern eeeee- |
| | | GACTTAAACT | dddddddddd-eeeee, with internucleoside linkages soooo- |
| | | | ssssssssss-ooss, and each C is a 5-methyl C |
| 364 | GYS1 | GATTTGCAAG | GATTTGCAAGTGACTCTCAA, with modification pattern eeeee- |
| | | TGACTCTCAA | dddddddddd-eeeee, with internucleoside linkages soooo- |
| | | | ssssssssss-ooss, and each C is a 5-methyl C |
| 365 | GYS1 | TGGATTCCCT | TGGATTCCCTCTGTAGATCA, with modification pattern eeeee- |
| | | CTGTAGATCA | dddddddddd-eeeee, with internucleoside linkages soooo- |
| | | | ssssssssss-ooss, and each C is a 5-methyl C |
| 366 | GYS1 | TGTCTCTAGC | TGTCTCTAGCTCTGACAACA, with modification pattern eeeee- |
| | | TCTGACAACA | dddddddddd-eeeee, with internucleoside linkages soooo- |
| | | | ssssssssss-ooss, and each C is a 5-methyl C |
| 367 | GYS1 | CCAGATGCTA | CCAGATGCTATTTCTAGATT, with modification pattern eeeee- |
| | | TTTCTAGATT | dddddddddd-eeeee, with internucleoside linkages soooo- |
| | | | ssssssssss-ooss, and each C is a 5-methyl C |
| 368 | GYS1 | ACTGCTGGAG | ACTGCTGGAGTCCCCAGCAA, with modification pattern eeeee- |
| | | TCCCCAGCAA | dddddddddd-eeeee, with internucleoside linkages soooo- |
| | | | ssssssssss-ooss, and each C is a 5-methyl C |
| 369 | PrP | GTGTCATAAT | Ges Teo Geo Teo mCeo Aeo Tds Ads Ads Tds Tds Tds |
| | | TTTCTTAGCT | Tds mCds Tds Tds Aeo Ges mCes Te |
| 370 | PrP | GTCATAATTT | Ges Teo mCeo Aeo Teo Aeo Ads Tds Tds Tds Tds mCds |
| | | TCTTAGCTAC | Tds Tds Ads Gds mCeo Tes Aes mCe |
| 371 | PrP | GCTTATTATT | Ges mCeo Teo Teo Aeo Teo Tds Ads Tds Tds mCds Ads Tds |
| | | CATGTTCTCC | Gds Tds Tds mCeo Tes mCes mCe |
| 372 | PrP | GTCATAATTT | Ges Teo mCeo Aeo Teo Ads Ads Tds Tds Tds Tds mCds |
| | | TCTTAGCTA | Tds Tds Aes Geo mCeo Tes Ae |
| 373 | PrP | ACGTCCATTT | Aes mCeo Geo Teo mCes mCds Ads Tds Tds Tds Tds |
| | | TCTGTGCTTT | mCds Tds Gds Tds Geo mCeo Tes Tes Te |
| 374 | GFAP | CAGTATTACC | mCes Aeo Geo Teo Aeo Teo Tds Ads mCds mCds Tds |
| | | TCTACTAGTC | mCds Tds Ads mCds Tds Aeo Ges Tes mCe |
| | LSD | | siRNA comprising: |
| | | | sense sequence: CCACGAGUCAAACCUUUAUTT (SEQ ID NO: |
| | | | 375) |
| | | | and antisense sequence: AUAAAGGUUUGACUCGUGGTT (SEQ ID |
| | | | NO: 376) |
| | LSD | | siRNA comprising: |
| | | | sense sequence: CACAAGGAAAGCUAGAAGATT (SEQ ID NO: |
| | | | 391) |
| | | | and antisense sequence: UCUUCUAGCUUUCCUUGUGTT (SEQ ID |
| | | | NO: 392) |
| 377 | UBE3A | ACCATTTTGA | Aes mCeo mCeo Aeo Teo Tds Tds Tds Gds Ads mCds |
| | | CCTTCTTAGC | mCds Tds Tds mCds Teo Teo Aes Ges mCe |
| 378 | UBE3A | CATCATGATC | mCes Aeo Teo mCeo Aeo Tds Gds Ads Tds mCds Tds |
| | | TTGGTAAGGC | Tds Gds Gds Tds Aeo Aeo Ges Ges mCe |
| 379 | UBE3A | TCACCATTTT | Tes mCeo Aeo mCeo mCeo Aeo Tds Tds Tds Tds Gds |
| | | GACCTTCTTA | Ads mCds mCds Tds Tds mCeo Tes Tes Ae |
| 380 | UBE3A | TTCACCATTI | Tes Teo mCeo Aeo mCeo mCeo Ads Tds Tds Tds Tds |
| | | TGACCTTCTT | Gds Ads mCds mCds Tds Teo mCes Tes Te |
| 381 | UBE3A | GCATACCCAG | Ges mCeo Aeo Teo Aeo mCeo mCds mCds Ads Gds Gds |
| | | GGTAGGATTC | Gds Tds Ads Gds Gds Aeo Tes Tes mCe |
| 382 | UBE3A | ACGCAATGTA | Aes mCeo Geo mCes Ads Ads Tds Gds Tds Ads Tds |
| | | TCAGGCAA | mCds Aeo Geo Geo mCes Aes Ae |
| 383 | UBE3A | TAGAGGTGAA | Tes Aes Ges Aes Ges Gds Tds Gds Ads Ads Gds Gds |
| | | GGCCAGGCAC | mCds mCds Ads Ges Ges mCes Aes mCe |
| 384 | UBE3A | GTACTCTTCC | Ges Tes Aes mCes Tes mCds Tds Tds mCds mCds Tds |
| | | TCAGTCATCC | mCds Ads Gds Tds mCes Aes Tes mCes mCe |
| 385 | UBE3A | TGTCAGTTTC | Tes Ges Tes mCes Aes Gds Tds Tds Tds mCds Tds |
| | | TCCCTGAACA | mCds mCds mCds Tds Ges Aes Aes mCes Ae |
| 386 | UBE3A | TAGAATGGCA | Tes Aes Ges Aes Aes Tds Gds Gds mCds Ads mCds Ads |
| | | CATCTCTTGG | Tds mCds Tds mCes Tes Tes Ges Ge |
| 387 | UBE3A | GTTTTCTTCC | Ges Tes Tes Tes Tes mCds Tds Tds mCds mCds Tds |
| | | TCCACAGTCT | mCds mCds Ads mCds Aes Ges Tes mCes Te |
| 388 | UBE3A | CTGGTGTCAA | mCes Tes Ges Ges Tes Gds Tds mCds Ads Ads mCds |
| | | CAAGCCAAAG | Ads Ads Gds mCds mCes Aes Aes Aes Ge |
| 389 | VLA-4 | CTGAGTCTGT | mCes Teo Geo Ads Gds Tds mCds Tds Gds Tds Tds Tds |
| | | TTTCCATTCT | Teo mCeo mCeo Aeo Teo Tes mCes Te |
| 390 | VLA-4 | CTGAGTCTGT | mCes Tes Ges Ads Gds Tds mCds Tds Gds Tds Tds Tds |
| | | TTTCCATTCT | Tes mCes mCes Aes Tes Tes mCes Te |

Abbreviations used in Table 18: mC = 5-methyl cytosine; e = 2′-O-methoxyethylribose modified
sugar; d = 2′-deoxyribose sugar; s = phosphorothioate internucleoside linkage; o =
phosphodiester internucleoside linkage. In each sequence listed in Table 18, each T may be
optionally and independently replaced with a U, and each U may be optionally and
independently replaced with a T.

Examples of small molecules useful for the treatment of other neurological disorders include:

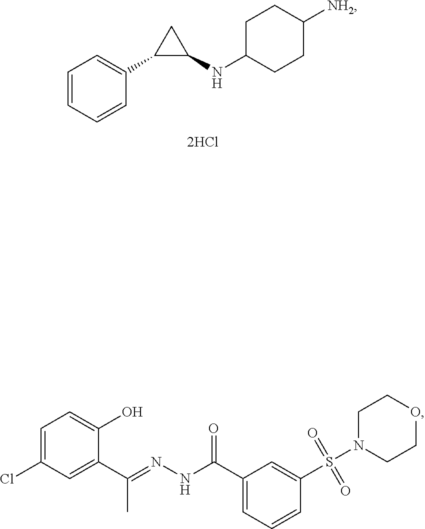 [SMILES: NC1CCC(N[C@@H]2C[C@H]2c2ccccc2)CC1 ; C/C(=N\NC(=O)c1cccc(S(=O)(=O)N2CCOCC2)c1)c1cc(Cl)ccc1O]

and pharmaceutically acceptable salts, co-crystals, tautomers, stereoisomers, solvates, hydrates, polymorphs, isotopically enriched compounds, and prodrugs thereof.

Examples of polypeptides useful for the treatment of other neurological disorders include antibodies, proteins, peptides, and enzymes.

### Molecular Payloads Targeting GYS1

The GYS1 gene, and mutations therein, are implicated in glycogen synthesis disorders, which can affect the CNS, as well as neurodegeneration, small fiber neuropathy, nociception-related phenotypes, Alexander disease, Angelman Syndrome, autism-spectrum disorders, retinitis pigmentosa, isolated macular dystrophy, and multiple sclerosis. Modulation of GYS1 expression and activity (e.g., by suppressing the expression of GYS1 or a mutant form thereof and/or activity of a wild-type or mutant form of a protein encoded by GYS1) therefore in some embodiments can have a therapeutic effect in subjects with a disorder described herein.

### Oligonucleotides

GYS1 expression and/or activity in some embodiments can be modulated by the use of oligonucleotides targeting GYS1 sequences.

In some embodiments, an oligonucleotide useful for the treatment of glycogen synthesis disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, e.g., targeting (e.g., directly or indirectly modulating the expression or activity of) GYS1, comprises a region of complementarity to a GYS1 transcript provided in Table 3, e.g., provided by any one of SEQ ID NOs: 605-607.

In some embodiments, examples of oligonucleotides useful for the treatment of glycogen synthesis disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, e.g., targeting (e.g., directly or indirectly modulating the expression or activity of) GYS1, are provided in Ahonen S, et al. “Gys1 antisense therapy rescues neuropathological bases of murine Lafora disease.” Brain. (2021) 144(10):2985-2993; and U.S. Patent Application Publication No. US20190194666A1, published Jun. 27, 2019, entitled “Modulation of gys1 expression”; the entire contents of each of which are herein incorporated by reference.

Certain oligonucleotides provided in this section may be useful in treating glycogen synthesis disorders by modulating the activity of genes and/or gene products other than GYS1 genes/gene products, such as PrP, GFAP, LSD, SCN9A, UBE3A, and/or VLA-4 genes/gene products.

### Polypeptides

GYS1 expression and/or activity in some embodiments can be modulated by the use of GYS1 polypeptides or polypeptides that can interact with GYS1 (e.g., to modulate its activity). In some embodiments, a polypeptide useful in the treatment of glycogen synthesis disorders is a peptide, a protein, an enzyme, or an antibody that modulates GYS1, e.g., by interacting with GYS1 or a protein encoded by GYS1.

### Small Molecules

GYS1 expression and/or activity in some embodiments can be modulated by the use of small molecules that can modulate SCN1A (e.g., to modulate its activity, or its expression). In some embodiments, a small molecule useful in the treatment of glycogen synthesis disorders is a small molecule that modulates GYS1, e.g., by interacting with GYS1 or a protein encoded by GYS1, by stimulating expression of GYS1, or by suppressing expression of GYS1.

In some embodiments, the small molecule is a pharmaceutically acceptable salt, co-crystal, tautomer, stereoisomer, solvate, hydrate, polymorph, isotopically enriched compound, or prodrug of a small molecule provided herein.

### Gene Therapies

GYS1 expression and/or activity in some embodiments can be modulated by the use of gene therapies that can modulate GYS1 (e.g., by delivery of nucleic acids encoding GYS1 or other molecules that interact with GYS1). In some embodiments, a gene therapy payload useful in the treatment of glycogen synthesis disorders is a payload that modulates GYS1, e.g., by interacting with GYS1 or a protein encoded by GYS1, by stimulating expression of GYS1 (such as by providing a molecule that encodes GYS1), or by suppressing expression of GYS1 (such as by providing a molecule that encodes a suppressor of GYS1).

### Molecular Payloads Targeting PrP

The PrP gene, and mutations therein, are implicated in various neurological disorders, including neurodegeneration, small fiber neuropathy, nociception-related phenotypes, Alexander disease, Angelman Syndrome, autism-spectrum disorders, retinitis pigmentosa, isolated macular dystrophy, and multiple sclerosis. Modulation of GYS1 expression and activity (e.g., by suppressing the expression of GYS1 or a mutant form thereof and/or activity of a wild-type or mutant form of a protein encoded by GYS1) therefore in some embodiments can have a therapeutic effect in subjects with a disorder described herein.

### Oligonucleotides

PrP expression and/or activity in some embodiments can be modulated by the use of oligonucleotides targeting PrP sequences.

In some embodiments, an oligonucleotide useful for the treatment of glycogen synthesis disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, e.g., targeting (e.g., directly or indirectly modulating the expression or activity of) PrP, comprises a region of complementarity to a PrP transcript provided in Table 3, e.g., provided by any one of SEQ ID NOs: 608-613.

In some embodiments, examples of oligonucleotides useful for the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, e.g., targeting (e.g., directly or indirectly modulating the expression or activity of) PrP, are provided in U.S. Patent Application Publication No. US20220025366A1, published Jan. 27, 2022, entitled “Compounds and methods for reducing prion expression”; U.S. Patent Application Publication No. US20210017513A1, published Jan. 21, 2021, entitled “Modified compounds and uses thereof”; U.S. Patent Application Publication No. US20130046007A1, published Feb. 21, 2013, entitled “Selective reduction of allelic variants”; and U.S. Patent Application Publication No. US20110269818A1, published Nov. 3, 2011, entitled “Modulation of prion expression”; the entire contents of each of which are herein incorporated by reference.

Certain oligonucleotides provided in this section may be useful in treating epilepsy by modulating the activity of genes and/or gene products gene products other than PrP genes/gene products, such as GYS1, GFAP, LSD, SCN9A, UBE3A, and/or VLA-4 genes/gene products.

### Polypeptides

PrP expression and/or activity in some embodiments can be modulated by the use of PrP polypeptides or polypeptides that can interact with PrP (e.g., to modulate its activity, its interaction with other biomolecules, and/or its aggregation).

In some embodiments, examples of polypeptides (e.g., peptides; proteins, such as enzymes; antibodies; etc.) useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis are provided in Rovis T L, Legname G. “Prion protein-specific antibodies-development, modes of action and therapeutics application.” Viruses. (2014) 6(10):3719-37; Sadowski M J, et al. “Anti-PrP Mab 6D11 suppresses PrP(Sc) replication in prion infected myeloid precursor line FDC-P1/22L and in the lymphoreticular system in vivo.” Neurobiol Dis. (2009) 34(2):267-78; U.S. Patent Application Publication No. US20190062442A1, published Feb. 28, 2019, entitled “Anti-PrP antibodies and uses thereof”; and International Patent Application Publication No. WO2006102099A2, published Sep. 28, 2006, entitled “Antibodies specific for human and bovine PrP”; the entire contents of each of which are herein incorporated by reference.

Certain polypeptides provided in this section may be useful in treating epilepsy by modulating the activity of genes and gene products other than PrP genes/gene products, such as GYS1, GFAP, LSD, SCN9A, UBE3A, and/or VLA-4 genes/gene products.

### Small Molecules

PrP expression and/or activity in some embodiments can be modulated by the use of small molecules that can modulate PrP (e.g., to modulate its expression, its activity, its interaction with other biomolecules, and/or its aggregation).

In some embodiments, examples of small molecules useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, are provided in Massignan T, et al. “A Small-Molecule Inhibitor of Prion Replication and Mutant Prion Protein Toxicity. ChemMedChem.” (2017) 12(16):1286-1292; the entire contents of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy by modulating the activity of genes and gene products other than PrP genes/gene products, such as GYS1, GFAP, LSD, SCN9A, UBE3A, and/or VLA-4 genes/gene products.

### Gene Therapies

PrP expression and/or activity in some embodiments can be modulated by the use of gene therapies that can modulate PrP (e.g., by delivery of nucleic acids encoding PrP or other molecules that interact with PrP).

In some embodiments, gene therapies, such as those involving administration of compounds encoding useful therapeutic agents, useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, are provided in U.S. Patent Application Publication No. US2021162072A1, published Jun. 3, 2021, entitled “Modified adeno-associated virus vectors and delivery thereof into the central nervous system”; the entire contents of each of which are herein incorporated by reference.

Certain gene therapies provided in this section may be useful in treating epilepsy by modulating the activity of genes and gene products other than PrP genes/gene products, such as GYS1, GFAP, LSD, SCN9A, UBE3A, and/or VLA-4 genes/gene products.

### Molecular Payloads Targeting GFAP

The GFAP gene, and mutations therein, are implicated in various neurological disorders, including neurodegeneration, small fiber neuropathy, nociception-related phenotypes, Alexander disease, Angelman Syndrome, autism-spectrum disorders, retinitis pigmentosa, isolated macular dystrophy, and multiple sclerosis. Modulation of GFAP expression and activity (e.g., by suppressing the expression of GFAP or a mutant form thereof and/or activity of a wild-type or mutant form of a protein encoded by GFAP) therefore in some embodiments can have a therapeutic effect in subjects with a disorder described herein.

### Oligonucleotides

GFAP expression and/or activity in some embodiments can be modulated by the use of oligonucleotides targeting GFAP sequences.

In some embodiments, an oligonucleotide useful for the treatment of glycogen synthesis disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, e.g., targeting (e.g., directly or indirectly modulating the expression or activity of) GFAP, comprises a region of complementarity to a GFAP transcript provided in Table 3, e.g., provided by any one of SEQ ID NOs: 614-617.

In some embodiments, examples of oligonucleotides useful for the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, e.g., targeting (e.g., directly or indirectly modulating the expression or activity of) GFAP, are provided in U.S. Patent Application Publication No. US20210017513A1, published Jan. 21, 2021, entitled “Modified compounds and uses thereof”; and U.S. Patent Application Publication No. US20130046007A1, published Feb. 21, 2013, entitled “Selective reduction of allelic variants”; the entire contents of each of which are herein incorporated by reference.

Certain oligonucleotides provided in this section may be useful in treating epilepsy by modulating the activity of genes and/or gene products gene products other than GFAP genes/gene products, such as GYS1, PrP, LSD, SCN9A, UBE3A, and/or VLA-4 genes/gene products.

### Polypeptides

GFAP expression and/or activity in some embodiments can be modulated by the use of GFAP polypeptides or polypeptides that can interact with GFAP (e.g., to modulate its activity).

In some embodiments, examples of polypeptides (e.g., peptides; proteins, such as enzymes; antibodies; etc.) useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis are provided in U.S. Patent Application Publication No. US20220054607A1, published Feb. 24, 2022, entitled “Glial fibrillary acidic protein targeting immuno- and aptamer-based-therapy for neuroinjury, neurodegeneration, neuro-disease, and neuro-repair”; U.S. Patent Application Publication No. US20220054607A1, published Feb. 24, 2022, entitled “Glial fibrillary acidic protein targeting immuno- and aptamer-based-therapy for neuroinjury, neurodegeneration, neuro-disease, and neuro-repair”; U.S. Patent Application Publication US20200165355A1, published May 28, 2020, entitled “Antibodies to ubiquitin c-terminal hydrolase l1 (uch-l1) and glial fibrillary acidic protein (gfap) and related methods”; and U.S. Patent Application Publication No. US20110250211A1, published Oct. 13, 2011, entitled “Variable domains of camelid heavy-chain antibodies directed against glial fibrillary acidic proteins”; the entire contents of each of which are herein incorporated by reference.

Certain polypeptides provided in this section may be useful in treating epilepsy by modulating the activity of genes and gene products other than GFAP genes/gene products, such as GYS1, PrP, LSD, SCN9A, UBE3A, and/or VLA-4 genes/gene products.

### Small Molecules

GFAP expression and/or activity in some embodiments can be modulated by the use of small molecules that can modulate GFAP (e.g., to modulate its activity, or its expression).

In some embodiments, examples of small molecules useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, are provided in Cho W, et al. “Drug screening to identify suppressors of GFAP expression.” Hum Mol Genet. (2010) 19(16):3169-78; the entire contents of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy by modulating the activity of genes and gene products other than GFAP genes/gene products, such as GYS1, PrP, LSD, SCN9A, UBE3A, and/or VLA-4 genes/gene products.

### Gene Therapies

GFAP expression and/or activity in some embodiments can be modulated by the use of gene therapies that can modulate GFAP (e.g., by delivery of nucleic acids encoding GFAP or other molecules that interact with GFAP).

In some embodiments, gene therapies, such as those involving administration of compounds encoding useful therapeutic agents, useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, are provided in U.S. Patent Application Publication No. US2021162072A1, published Jun. 3, 2021, entitled “Modified adeno-associated virus vectors and delivery thereof into the central nervous system”; the entire contents of each of which are herein incorporated by reference.

Certain gene therapies provided in this section may be useful in treating epilepsy by modulating the activity of genes and gene products other than GFAP genes/gene products, such as GYS1, PrP, LSD, SCN9A, UBE3A, and/or VLA-4 genes/gene products.

### Molecular Payloads Targeting LSD

The LSD genes, and mutations therein, are implicated in various neurological disorders, including neurodegeneration, small fiber neuropathy, nociception-related phenotypes, Alexander disease, Angelman Syndrome, autism-spectrum disorders, retinitis pigmentosa, isolated macular dystrophy, and multiple sclerosis. Modulation of LSD expression and activity (e.g., by suppressing the expression of an LSD gene or a mutant form thereof and/or activity of a wild-type or mutant form of a protein encoded by an LSD gene) therefore in some embodiments can have a therapeutic effect in subjects with a disorder described herein.

### Oligonucleotides

LSD expression and/or activity in some embodiments can be modulated by the use of oligonucleotides targeting LSD sequences.

In some embodiments, an oligonucleotide useful for the treatment of glycogen synthesis disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, e.g., targeting (e.g., directly or indirectly modulating the expression or activity of) LSD, comprises a region of complementarity to an LSD transcript provided in Table 3, e.g., provided by any one of SEQ ID NOs: 618-626.

In some embodiments, examples of oligonucleotides useful for the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, e.g., targeting (e.g., directly or indirectly modulating the expression or activity of) LSD, are provided in Sobczak M, et al. “LSD1 Facilitates Pro-Inflammatory Polarization of Macrophages by Repressing Catalase.” Cells. (2021) 10(9):2465; Zou Z K, et al. “Silencing of LSD1 gene modulates histone methylation and acetylation and induces the apoptosis of JeKo-1 and MOLT-4 cells.” Int J Mol Med. (2017) 40(2):319-328; and Kong L L, et al. “Downregulation of LSD1 suppresses the proliferation, tumorigenicity and invasion of papillary thyroid carcinoma K1 cells.” Oncol Lett. (2016) 11(4):2475-2480; the entire contents of each of which are herein incorporated by reference.

Certain oligonucleotides provided in this section may be useful in treating epilepsy by modulating the activity of genes and/or gene products gene products other than LSD genes/gene products, such as GYS1, PrP, GFAP, SCN9A, UBE3A, and/or VLA-4 genes/gene products.

### Polypeptides

LSD expression and/or activity in some embodiments can be modulated by the use of LSD polypeptides or polypeptides that can interact with a protein encoded by an LSD gene (e.g., to modulate its activity).

In some embodiments, examples of polypeptides (e.g., peptides; proteins, such as enzymes; antibodies; etc.) useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis are provided in Maes T, et al. “ORY-1001, a Potent and Selective Covalent KDM1A Inhibitor, for the Treatment of Acute Leukemia.” Cancer Cell. (2018) 33(3):495-511.e12; the entire contents of which are herein incorporated by reference.

Certain polypeptides provided in this section may be useful in treating epilepsy by modulating the activity of genes and gene products other than LSD genes/gene products, such as GYS1, PrP, GFAP, SCN9A, UBE3A, and/or VLA-4 genes/gene products.

### Small Molecules

LSD expression and/or activity in some embodiments can be modulated by the use of small molecules that can modulate LSD (e.g., to modulate its activity, or its expression).

In some embodiments, examples of small molecules useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, are provided in Song Z, et al. “Discovery of the antitumor activities of a potent DCN1 inhibitor compound 383 targeting LSD1 in gastric cancer.” Eur J Pharmacol. (2022) 916:174725; Maes T, et al. “ORY-1001, a Potent and Selective Covalent KDM1A Inhibitor, for the Treatment of Acute Leukemia.” Cancer Cell. (2018) 33(3):495-511.e12; U.S. Patent Application Publication No. US20190092724A1, published Mar. 28, 2019, entitled “Cyano-substituted indole compounds and uses thereof as lsd1 inhibitors”; U.S. Patent Application Publication No. US20160289238A1, published Oct. 6, 2016, entitled “Heterocyclic compounds as lsd1 inhibitors”; U.S. Patent Application Publication No. US20160009711A1, published Jan. 14, 2016, entitled “Triazolopyridines and triazolopyrazines as lsd1 inhibitors”; U.S. Patent Application Publication No. US20160009721A1, published Jan. 14, 2016, entitled “Triazolopyridines and triazolopyrazines as lsd1 inhibitors”; U.S. Patent Application Publication No. US20160009712A1, published Jan. 14, 2016, entitled “Imidazopyridines and imidazopyrazines as lsd1 inhibitors”; U.S. Patent Application Publication No. US20150225401A1, published Aug. 13, 2015, entitled “Cyclopropylamines as lsd1 inhibitors”; U.S. Patent Application Publication No. US20150225379A1, published Aug. 13, 2015, entitled “Cyclopropylamines as lsd1 inhibitors”; U.S. Patent Application Publication No. US20160120862A1, published May 5, 2016, entitled “Suicidal lsd1 inhibitors targeting sox2-expressing cancer cells”; U.S. Patent Application Publication No. US20160081947A1, published Mar. 24, 2016, entitled “Selective lsd1 and dual lsd1/mao-b inhibitors for modulating diseases associated with alterations in protein conformation”; and U.S. Patent Application Publication No. US20130231342A1, published Sep. 5, 2013, entitled “Arylcyclopropylamine based demethylase inhibitors of lsd1 and their medical use”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy by modulating the activity of genes and gene products other than LSD genes/gene products, such as GYS1, PrP, GFAP, SCN9A, UBE3A, and/or VLA-4 genes/gene products.

### Gene Therapies

LSD expression and/or activity in some embodiments can be modulated by the use of gene therapies that can modulate LSD (e.g., by delivery of nucleic acids encoding LSD or other molecules that interact with LSD).

In some embodiments, gene therapies, such as those involving administration of compounds encoding useful therapeutic agents, useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, are provided in International Patent Application Publication No. WO2020210542A1, published Oct. 15, 2020, entitled “Long-lasting analgesia via targeted in vivo epigenetic repression”; the entire contents of which are herein incorporated by reference.

Certain gene therapies provided in this section may be useful in treating epilepsy by modulating the activity of genes and gene products other than LSD genes/gene products, such as GYS1, PrP, GFAP, SCN9A, UBE3A, and/or VLA-4 genes/gene products.

### Molecular Payloads Targeting SCN9A

The SCN9A gene, and mutations therein, are implicated in neurodegeneration, small fiber neuropathy, nociception-related phenotypes, Alexander disease, Angelman Syndrome, autism-spectrum disorders, retinitis pigmentosa, isolated macular dystrophy, and multiple sclerosis. Modulation of SCN9A expression and activity (e.g., by suppressing the expression of SCN9A or a mutant form thereof and/or activity of a wild-type or mutant form of a protein encoded by SCN9A) therefore in some embodiments can have a therapeutic effect in subjects with epilepsy, pain disorders, and/or other neurological disorders described herein.

### Oligonucleotides

SCN9A expression and/or activity in some embodiments can be modulated by the use of oligonucleotides targeting SCN9A sequences.

In some embodiments, an oligonucleotide useful for the treatment of glycogen synthesis disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, e.g., targeting (e.g., directly or indirectly modulating the expression or activity of) SCN9A, comprises a region of complementarity to a SCN9A transcript provided in Table 3, e.g., provided by any one of SEQ ID NOs: 573-580.

In some embodiments, examples of oligonucleotides useful for the treatment of neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, e.g., targeting (e.g., directly or indirectly modulating the expression or activity of) SCN9A, are provided in U.S. Patent Application Publication No. US20210238608A1, published Aug. 5, 2021, entitled “Oligonucleotides for modulating scn9a expression”; and U.S. Patent Application Publication No. US20190218255A1, published Jul. 18, 2019, entitled “Scn9a antisense oligonucleotides”; the entire contents of each of which are herein incorporated by reference.

Certain oligonucleotides provided in this section may be useful in treating neurodegeneration, small fiber neuropathy, nociception-related phenotypes, Alexander disease, Angelman Syndrome, autism-spectrum disorders, retinitis pigmentosa, isolated macular dystrophy, and multiple sclerosis by modulating the activity of genes and/or gene products other than SCN9A genes/gene products, such as GYS1, LSD, PrP, GFAP, UBE3A, and/or VLA-4 genes/gene products.

### Polypeptides

SCN9A expression and/or activity in some embodiments can be modulated by the use of SCN8A polypeptides or polypeptides that can interact with SCN8A (e.g., to modulate its activity).

In some embodiments, examples of polypeptides (e.g., peptides; proteins, such as enzymes; antibodies; etc.) useful for the treatment of neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, are provided in U.S. Patent Application Publication No. US20160024208A1, published Jan. 28, 2016, entitled “Human antibodies to nav1.7”; U.S. Patent Application Publication No. US20120259096A1, published Oct. 11, 2012, entitled “Antibodies to the E1 extracellular loop of ion channels”; U.S. Patent Application Publication No. US20140073577A1, published Mar. 13, 2014, entitled “Potent and selective inhibitors of nav1.3 and nav1.7”; U.S. Pat. No. 10,344,060B2, published Jul. 9, 2019, entitled “Potent and selective inhibitors of Nav1.7”; the entire contents of each of which are herein incorporated by reference.

Certain polypeptides provided in this section may be useful in treating neurodegeneration, small fiber neuropathy, nociception-related phenotypes, Alexander disease, Angelman Syndrome, autism-spectrum disorders, retinitis pigmentosa, isolated macular dystrophy, and multiple sclerosis by modulating the activity of genes and/or gene products other than SCN9A genes/gene products, such as GYS1, LSD, PrP, GFAP, UBE3A, and/or VLA-4 genes/gene products.

### Small Molecules

SCN9A expression and/or activity in some embodiments can be modulated by the use of small molecules that can modulate SCN9A (e.g., to modulate its activity, or its expression).

In some embodiments, examples of small molecules useful for the treatment of neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, are provided in U.S. Patent Application Publication No. US20220073463A1, published Mar. 10, 2022, entitled “Formulations of t-type calcium channel modulators and methods of use thereof”; U.S. Patent Application Publication No. US20190160078A1, published May 30, 2019, entitled “Ganaxolone for use in treating genetic epileptic disorders”; U.S. Patent Application Publication No. US20190091231A1, published Mar. 28, 2019, entitled “Methods of treating developmental disorders and/or seizure disorders with etifoxine”, U.S. Patent Application Publication No. US20200085838A1, published Mar. 19, 2020, entitled “Methods of treating epilepsy and neurodevelopmental disorders”; and U.S. Patent Application Publication No. US20180296501A1, published Oct. 18, 2018, entitled “Methods of treating developmental encephalopathies”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating neurodegeneration, small fiber neuropathy, nociception-related phenotypes, Alexander disease, Angelman Syndrome, autism-spectrum disorders, retinitis pigmentosa, isolated macular dystrophy, and multiple sclerosis by modulating the activity of genes and/or gene products other than SCN9A genes/gene products, such as GYS1, LSD, PrP, GFAP, UBE3A, and/or VLA-4 genes/gene products.

### Gene Therapies

SCN9A expression and/or activity in some embodiments can be modulated by the use of gene therapies that can modulate SCN9A (e.g., by delivery of nucleic acids encoding SCN9A or other molecules that interact with SCN9A).

In some embodiments, gene therapies, such as those involving administration of compounds encoding useful therapeutic agents, useful for the treatment of neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, are provided in U.S. Patent Application Publication No. US2021162072A1, published Jun. 3, 2021, entitled “Modified adeno-associated virus vectors and delivery thereof into the central nervous system”; International Patent Application Publication No. WO2020210542A1, published Oct. 15, 2020, entitled “Long-lasting analgesia via targeted in vivo epigenetic repression”; the entire contents of each of which are herein incorporated by reference.

Certain gene therapies provided in this section may be useful in treating neurodegeneration, small fiber neuropathy, nociception-related phenotypes, Alexander disease, Angelman Syndrome, autism-spectrum disorders, retinitis pigmentosa, isolated macular dystrophy, and multiple sclerosis by modulating the activity of genes and/or gene products other than SCN9A genes/gene products, such as GYS1, LSD, PrP, GFAP, UBE3A, and/or VLA-4 genes/gene products.

### Molecular Payloads Targeting UBE3A

The UBE3A gene, and mutations therein, are implicated in various neurological disorders, including neurodegeneration, small fiber neuropathy, nociception-related phenotypes, Alexander disease, Angelman Syndrome, autism-spectrum disorders, retinitis pigmentosa, isolated macular dystrophy, and multiple sclerosis. Modulation of UBE3A expression and activity (e.g., by suppressing the expression of UBE3A or a mutant form thereof and/or activity of a wild-type or mutant form of a protein encoded by UBE3A) therefore in some embodiments can have a therapeutic effect in subjects with a disorder described herein.

### Oligonucleotides

UBE3A expression and/or activity in some embodiments can be modulated by the use of oligonucleotides targeting UBE3A sequences.

In some embodiments, an oligonucleotide useful for the treatment of glycogen synthesis disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, e.g., targeting (e.g., directly or indirectly modulating the expression or activity of) UBE3A, comprises a region of complementarity to a UBE3A transcript provided in Table 3, e.g., provided by any one of SEQ ID NOs: 627-700.

In some embodiments, examples of oligonucleotides useful for the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, e.g., targeting (e.g., directly or indirectly modulating the expression or activity of) UBE3A, are provided in U.S. Patent Application Publication No. US20210277397A1, published Sep. 9, 2021, entitled “Compounds and methods for modulating ube3a-ats”; U.S. Patent Application Publication No. US20200370046A1 published Nov. 26, 2020, entitled “Angelman syndrome antisense treatment”; U.S. Patent Application Publication No. US20210017513A1, published Jan. 21, 2021, entitled “Modified compounds and uses thereof”; and U.S. Patent Application Publication No. US20170191064A1, published Jul. 6, 2017, entitled “Oligonucleotides for inducing paternal ube3a expression”; the entire contents of each of which are herein incorporated by reference.

Certain oligonucleotides provided in this section may be useful in treating epilepsy by modulating the activity of genes and/or gene products gene products other than UBE3A genes/gene products, such as GYS1, PrP, GFAP, LSD, SCN9A, and/or VLA-4 genes/gene products.

### Polypeptides

UBE3A expression and/or activity in some embodiments can be modulated by the use of UBE3A polypeptides or polypeptides that can interact with UBE3A (e.g., to modulate its activity).

In some embodiments, examples of polypeptides (e.g., peptides; proteins, such as enzymes; antibodies; etc.) useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis are provided in Sen D, et al. “Evaluation of UBE3A antibodies in mice and human cerebral organoids.” Sci Rep. (2021) 11(1):6323; the entire contents of which are herein incorporated by reference.

Certain polypeptides provided in this section may be useful in treating epilepsy by modulating the activity of genes and gene products other than UBE3A genes/gene products, such as GYS1, PrP, GFAP, LSD, SCN9A, and/or VLA-4 genes/gene products.

### Small Molecules

UBE3A expression and/or activity in some embodiments can be modulated by the use of small molecules that can modulate UBE3A (e.g., to modulate its activity, or its expression).

In some embodiments, examples of small molecules useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, are provided in Offensperger F, et al. “Identification of Small-Molecule Activators of the Ubiquitin Ligase E6AP/UBE3A and Angelman Syndrome-Derived E6AP/UBE3A Variants.” Cell Chem Biol. (2020) 27(12):1510-1520.e6; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy by modulating the activity of genes and gene products other than UBE3A genes/gene products, such as GYS1, PrP, GFAP, LSD, SCN9A, and/or VLA-4 genes/gene products.

### Gene Therapies

UBE3A expression and/or activity in some embodiments can be modulated by the use of gene therapies that can modulate UBE3A (e.g., by delivery of nucleic acids encoding UBE3A or other molecules that interact with UBE3A).

In some embodiments, gene therapies, such as those involving administration of a compounds encoding useful therapeutic agents, useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, are provided in International Patent Application Publication No. WO2022119890A1, published Jun. 9, 2022, entitled “Compositions and uses thereof for treatment of Angelman syndrome”; U.S. Patent Application Publication No. US20220152223A1, published May 19, 2022, entitled “Vector and method for treating Angelman syndrome”; U.S. Patent Application Publication No. US20210162072A1, published Jun. 3, 2021, entitled “Modified adeno-associated virus vectors and delivery thereof into the central nervous system”; International Patent Application Publication No. WO2021035181A1, published Feb. 25, 2021, entitled “Ube3a for the treatment of Angelman syndrome”; the entire contents of each of which are herein incorporated by reference.

Certain gene therapies provided in this section may be useful in treating epilepsy by modulating the activity of genes and gene products other than UBE3A genes/gene products, such as GYS1, PrP, GFAP, LSD, SCN9A, and/or VLA-4 genes/gene products.

### Molecular Payloads Targeting VLA-4

The VLA-4 gene, and mutations therein, are implicated in various neurological disorders, including neurodegeneration, small fiber neuropathy, nociception-related phenotypes, Alexander disease, Angelman Syndrome, autism-spectrum disorders, retinitis pigmentosa, isolated macular dystrophy, and multiple sclerosis. Modulation of VLA-4 expression and activity (e.g., by suppressing the expression of VLA-4 or a mutant form thereof and/or activity of a wild-type or mutant form of a protein encoded by VLA-4) therefore in some embodiments can have a therapeutic effect in subjects with a disorder described herein.

### Oligonucleotides

VLA-4 expression and/or activity in some embodiments can be modulated by the use of oligonucleotides targeting VLA-4 sequences.

In some embodiments, an oligonucleotide useful for the treatment of glycogen synthesis disorders, neurodegeneration, small fiber neuropathy, nociception-related phenotypes, Alexander disease, Angelman Syndrome, autism-spectrum disorders, retinitis pigmentosa, isolated macular dystrophy, and/or multiple sclerosis, e.g., targeting (e.g., directly or indirectly modulating the expression or activity of) VLA-4, comprises a region of complementarity to a VLA-4 transcript provided in Table 3, e.g., provided by any one of SEQ ID NOs: 701-702.

In some embodiments, examples of oligonucleotides useful for the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, e.g., targeting (e.g., directly or indirectly modulating the expression or activity of) VLA-4, are provided in U.S. Patent Application Publication No. US20210079391A1, published Mar. 18, 2021; U.S. Patent Application Publication No. US20200095587A1, published Mar. 26, 2020, entitled “Therapeutic uses and methods”; and U.S. Patent Application Publication No. US20090029931A1, published Jan. 29, 2009, entitled “Antisense modulation of integrin alpha4 expression”; the entire contents of each of which are herein incorporated by reference.

Certain oligonucleotides provided in this section may be useful in treating epilepsy by modulating the activity of genes and/or gene products gene products other than VLA-4 genes/gene products, such as GYS1, PrP, GFAP, LSD, SCN9A, and/or UBE3A genes/gene products.

### Polypeptides

VLA-4 expression and/or activity in some embodiments can be modulated by the use of VLA-4 polypeptides or polypeptides that can interact with VLA-4 (e.g., to modulate its activity).

In some embodiments, examples of polypeptides (e.g., peptides; proteins, such as enzymes; antibodies; etc.) useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis are provided in U.S. Patent Application Publication No. US20200166521A1, published May 28, 2020, entitled “Methods of treating inflammatory and autoimmune diseases with natalizumab”; U.S. Patent Application Publication No. US20120022236A1, published Jan. 26, 2012, entitled “Recombinant Anti-VLA4 Antibody Molecules”; U.S. Patent Application Publication No. US20070231319A1, published Oct. 4, 2007, entitled “Methods of treating inflammatory and autoimmune diseases with natalizumab”; and U.S. Patent Application Publication No. US20040009169A1, published Jan. 15, 2004, entitled “Administration of agents for the treatment of inflammation”; the entire contents of each of which are herein incorporated by reference.

Certain polypeptides provided in this section may be useful in treating epilepsy by modulating the activity of genes and gene products other than VLA-4 genes/gene products, such as GYS1, PrP, GFAP, LSD, SCN9A, and/or UBE3A genes/gene products.

### Small Molecules

VLA-4 expression and/or activity in some embodiments can be modulated by the use of small molecules that can modulate VLA-4 (e.g., to modulate its activity, or its expression).

In some embodiments, examples of small molecules useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, are provided in Chen Y H, et al. “Small-molecule antagonist of VLA-4 (GW559090) attenuated neuro-inflammation by targeting Th17 cell trafficking across the blood-retinal barrier in experimental autoimmune uveitis.” J Neuroinflammation. (2021) 18(1):49; Baiula M, et al. “Novel Ligands Targeting a401 Integrin: Therapeutic Applications and Perspectives.” Front Chem. (2019) 7:489; and Ramirez P, et al. “BIO5192, a small molecule inhibitor of VLA-4, mobilizes hematopoietic stem and progenitor cells.” Blood. (2009) 114(7):1340-3; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating epilepsy by modulating the activity of genes and gene products other than VLA-4 genes/gene products, such as GYS1, PrP, GFAP, LSD, SCN9A, and/or UBE3A genes/gene products.

### Gene Therapies

VLA-4 expression and/or activity in some embodiments can be modulated by the use of gene therapies that can modulate VLA-4 (e.g., by delivery of nucleic acids encoding VLA-4 or other molecules that interact with VLA-4).

In some embodiments, gene therapies, such as those involving administration of compounds encoding useful therapeutic agents, useful in the treatment of neurological disorders, such as, but not limited to, neurodegeneration, small fiber neuropathy, nociception-related phenotypes, Alexander disease, Angelman Syndrome, autism-spectrum disorders, retinitis pigmentosa, isolated macular dystrophy, and multiple sclerosis, are provided in Darzi L, et al. “The silencing effect of miR-30a on ITGA4 gene expression in vitro: an approach for gene therapy.” Res Pharm Sci. (2017) 12(6):456-464; the entire contents of which are herein incorporated by reference.

Certain gene therapies provided in this section may be useful in treating epilepsy by modulating the activity of genes and gene products other than VLA-4 genes/gene products, such as GYS1, PrP, GFAP, LSD, SCN9A, and/or UBE3A genes/gene products.

### Molecular Payloads for the Treatment of GM1 Gangliosidosis

Various molecular payloads may be useful in the treatment of GM1 gangliosidosis, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of GM1 gangliosidosis may include, in some embodiments, molecular payloads which modulate (e.g., increase or decrease) expression or activity of GLB1. In some embodiments, treatment of GM1 gangliosidosis comprises enzyme replacement therapy (ERT), comprising delivery to a cell or tissue of a subject in need thereof a functional form of a GLB1 protein or a functional fragment thereof. In some embodiments, treatment of GM1 gangliosidosis comprises enzyme replacement therapy (ERT), comprising delivery to a cell or tissue of a subject in need thereof a nucleic acid (e.g., mRNA or cDNA) encoding a functional form of a galactosidase beta 1 protein or functional fragment thereof.

Examples of small molecules useful for the treatment of GM1 gangliosidosis include:

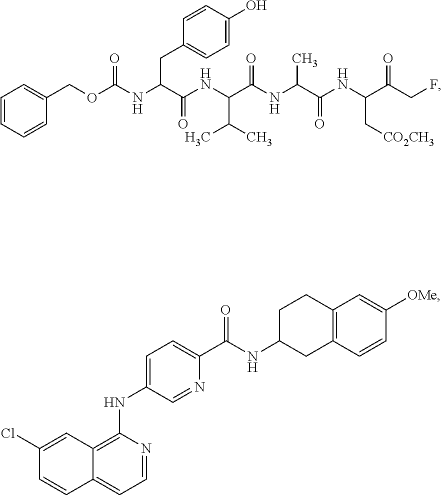 [SMILES: COC(=O)CC(NC(=O)C(C)NC(=O)C(NC(=O)C(Cc1ccc(O)cc1)NC(=O)OCc1ccccc1)C(C)C)C(=O)CF ; COc1ccc2c(c1)CCC(NC(=O)c1ccc(Nc3nccc4ccc(Cl)cc34)cn1)C2]

and pharmaceutically acceptable salts, co-crystals, tautomers, stereoisomers, solvates, hydrates, polymorphs, isotopically enriched compounds, and prodrugs thereof.

Examples of polypeptides useful for the treatment of GM1 gangliosidosis include antibodies, proteins, peptides, and enzymes. In some embodiments, a polypeptide useful for the treatment of GM1 gangliosidosis comprises an amino acid sequence having at least 70% sequence identity (e.g., at least 70%, 75%, 80%, 85%, 90%, 95%, 96%, 97%, 98%, 99% or 100%) to an amino acid sequence selected from:

(SEQ ID NO: 10301)
MPGFLVRILLLLLVLLLLGPTRGLRNATQRMFEIDYSRDSFLKDGQPFR
YISGSIHYSRVPRFYWKDRLLKMKMAGLNAIQTYVPWNFHEPWPGQYQF
SEDHDVEYFLRLAHELGLLVILRPGPYICAEWEMGGLPAWLLEKESILL
RSSDPDYLAAVDKWLGVLLPKMKPLLYQNGGPVITVQVENEYGSYFACD
FDYLRFLQKRFRHHLGDDVVLFTTDGAHKTFLKCGALQGLYTTVDFGTG
SNITDAFLSQRKCEPKGPLINSEFYTGWLDHWGQPHSTIKTEAVASSLY
DILARGASVNLYMFIGGTNFAYWNGANSPYAAQPTSYDYDAPLSEAGDL
TEKYFALRNIIQKFEKVPEGPIPPSTPKFAYGKVTLEKLKTVGAALDIL
CPSGPIKSLYPLTFIQVKQHYGFVLYRTTLPQDCSNPAPLSSPLNGVHD
RAYVAVDGIPQGVLERNNVITLNITGKAGATLDLLVENMGRVNYGAYIN
DFKGLVSNLTLSSNILTDWTIFPLDTEDAVRSHLGGWGHRDSGHHDEAW
AHNSSNYTLPAFYMGNFSIPSGIPDLPQDTFIQFPGWTKGQVWINGFNL
GRYWPARGPQLTLFVPQHILMTSAPNTITVLELEWAPCSSDDPELCAVT
FVDRPVIGSSVTYDHPSKPVEKRLMPPPPQKNKDSWLDHV;
or
(SEQ ID NO: 1031)
LRNATQRMFEIDYSRDSFLKDGQPFRYISGSIHYSRVPRFYWKDRLLKM
KMAGLNAIQTYVPWNFHEPWPGQYQFSEDHDVEYFLRLAHELGLLVILR
PGPYICAEWEMGGLPAWLLEKESILLRSSDPDYLAAVDKWLGVLLPKMK
PLLYQNGGPVITVQVENEYGSYFACDFDYLRFLQKRFRHHLGDDVVLFT
TDGAHKTFLKCGALQGLYTTVDFGTGSNITDAFLSQRKCEPKGPLINSE
FYTGWLDHWGQPHSTIKTEAVASSLYDILARGASVNLYMFIGGINFAYW
NGANSPYAAQPTSYDYDAPLSEAGDLTEKYFALRNIIQKFEKVPEGPIP
PSTPKFAYGKVTLEKLKTVGAALDILCPSGPIKSLYPLTFIQVKQHYGF
VLYRTTLPQDCSNPAPLSSPLNGVHDRAYVAVDGIPQGVLERNNVITLN
ITGKAGATLDLLVENMGRVNYGAYINDFKGLVSNLTLSSNILTDWTIFP
LDTEDAVRSHLGGWGHRDSGHHDEAWAHNSSNYTLPAFYMGNFSIPSGI
PDLPQDTFIQFPGWTKGQVWINGFNLGRYWPARGPQLTLFVPQHILMTS
APNTITVLELEWAPCSSDDPELCAVTFVDRPVIGSSVTYDHPSKPVEKR
LMPPPPQKNKDSWLDHV.

Examples of gene therapy payloads useful for the treatment of GM1 gangliosidosis include: nucleic acids (e.g., mRNA, cDNA, plasmid DNA, etc.), such as those encoding a functional GLB1 protein or a functional fragment thereof; viral vectors (e.g., AAV, lentivirus, etc.); etc. In some embodiments, a gene therapy payload useful for the treatment of GM1 gangliosidosis comprises a nucleic acid comprising a nucleobase sequence having at least 70% sequence identity (e.g., at least 70%, 75%, 80%, 85%, 90%, 95%, 96%, 97%, 98%, 99% or 100%) to a nucleobase sequence selected from:

(SEQ ID NO: 1032)
atgccggggttcctggttcgcatcctccttctgctgctggttctgctgcttctgggccctacgcgcggct
tgcgcaatgccacccagaggatgtttgaaattgactatagccgggactccttcctcaaggatggccagccatttcg
ctacatctcaggaagcattcactactcccgtgtgccccgcttctactggaaggaccggctgctgaagatgaagatg
gctgggctgaacgccatccagacgtatgtgccctggaactttcatgagccctggccaggacagtaccagttttctg
aggaccatgatgtggaatattttcttcggctggctcatgagctgggactgctggttatcctgaggcccgggcccta
catctgtgcagagtgggaaatgggaggattacctgcttggctgctagagaaagagtctattcttctccgctcctcc
gacccagattacctggcagctgtggacaagtggttgggagtccttctgcccaagatgaagcctctcctctatcaga
atggagggccagttataacagtgcaggttgaaaatgaatatggcagctactttgcctgtgattttgactacctgcg
cttcctgcagaagcgctttcgccaccatctgggggatgatgtggttctgtttaccactgatggagcacataaaaca
ttcctgaaatgtggggccctgcagggcctctacaccacggtggactttggaacaggcagcaacatcacagatgctt
tcctaagccagaggaagtgtgagcccaaaggacccttgatcaattctgaattctatactggctggctagatcactg
gggccaacctcactccacaatcaagaccgaagcagtggcttcctccctctatgatatacttgcccgtggggcgagt
gtgaacttgtacatgtttataggtgggaccaattttgcctattggaatggggccaactcaccctatgcagcacagc
ccaccagctacgactatgatgccccactgagtgaggctggggacctcactgagaagtattttgctctgcgaaacat
catccagaagtttgaaaaagtaccagaaggtcctatccctccatctacaccaaagtttgcatatggaaaggtcact
ttggaaaagttaaagacagtgggagcagctctggacattctgtgtccctctgggcccatcaaaagcctttatccct
tgacatttatccaggtgaaacagcattatgggtttgtgctgtaccggacaacacttcctcaagattgcagcaaccc
agcacctctctcttcacccctcaatggagtccacgatcgagcatatgttgctgtggatgggatcccccagggagtc
cttgagcgaaacaatgtgatcactctgaacataacagggaaagctggagccactctggaccttctggtagagaaca
tgggacgtgtgaactatggtgcatatatcaacgattttaagggtttggtttctaacctgactctcagttccaatat
cctcacggactggacgatctttccactggacactgaggatgcagtgcgcagccacctggggggctggggacaccgt
gacagtggccaccatgatgaagcctgggcccacaactcatccaactacacgctcccggccttttatatggggaact
tctccattcccagtgggatcccagacttgccccaggacacctttatccagtttcctggatggaccaagggccaggt
ctggattaatggctttaaccttggccgctattggccagcccggggccctcagttgaccttgtttgtgccccagcac
atcctgatgacctcggccccaaacaccatcaccgtgctggaactggagtgggcaccctgcagcagtgatgatccag
aactatgtgctgtgacgttcgtggacaggccagttattggctcatctgtgacctacgatcatccctccaaacctgt
tgaaaaaagactcatgcccccacccccgcaaaaaaacaaagattcatggctggaccatgtatga;
or
(SEQ ID NO: 1033)
atgccgggctttctggtgcgcattctgctgctgctgctggtgctgctgctgctgggcccgacccgcggcc
tgcgcaacgcgacccagcgcatgtttgaaattgattatagccgcgatagctttctgaaagatggccagccgtttcg
ctatattagcggcagcattcattatagccgcgtgccgcgcttttattggaaagatcgcctgctgaaaatgaaaatg
gcgggcctgaacgcgattcagacctatgtgccgtggaactttcatgaaccgtggccgggccagtatcagtttagcg
aagatcatgatgtggaatattttctgcgcctggcgcatgaactgggcctgctggtgattctgcgcccgggcccgta
tatttgcgcggaatgggaaatgggcggcctgccggcgtggctgctggaaaaagaaagcattctgctgcgcagcagc
gatccggattatctggcggcggtggataaatggctgggcgtgctgctgccgaaaatgaaaccgctgctgtatcaga
acggcggcccggtgattaccgtgcaggtggaaaacgaatatggcagctattttgcgtgcgattttgattatctgcg
ctttctgcagaaacgctttcgccatcatctgggcgatgatgtggtgctgtttaccaccgatggcgcgcataaaacc
tttctgaaatgcggcgcgctgcagggcctgtataccaccgtggattttggcaccggcagcaacattaccgatgcgt
ttctgagccagcgcaaatgcgaaccgaaaggcccgctgattaacagcgaattttataccggctggctggatcattg
gggccagccgcatagcaccattaaaaccgaagcggtggcgagcagcctgtatgatattctggcgcgcggcgcgagc
gtgaacctgtatatgtttattggcggcaccaactttgcgtattggaacggcgcgaacagcccgtatgcggcgcagc
cgaccagctatgattatgatgcgccgctgagcgaagcgggcgatctgaccgaaaaatattttgcgctgcgcaacat
tattcagaaatttgaaaaagtgccggaaggcccgattccgccgagcaccccgaaatttgcgtatggcaaagtgacc
ctggaaaaactgaaaaccgtgggcgcggcgctggatattctgtgcccgagcggcccgattaaaagcctgtatccgc
tgacctttattcaggtgaaacagcattatggctttgtgctgtatcgcaccaccctgccgcaggattgcagcaaccc
ggcgccgctgagcagcccgctgaacggcgtgcatgatcgcgcgtatgtggcggtggatggcattccgcagggcgtg
ctggaacgcaacaacgtgattaccctgaacattaccggcaaagcgggcgcgaccctggatctgctggtggaaaaca
tgggccgcgtgaactatggcgcgtatattaacgattttaaaggcctggtgagcaacctgaccctgagcagcaacat
tctgaccgattggaccatttttccgctggataccgaagatgcggtgcgcagccatctgggcggctggggccatcgc
gatagcggccatcatgatgaagcgtgggcgcataacagcagcaactataccctgccggcgttttatatgggcaact
ttagcattccgagcggcattccggatctgccgcaggatacctttattcagtttccgggctggaccaaaggccaggt
gtggattaacggctttaacctgggccgctattggccggcgcgcggcccgcagctgaccctgtttgtgccgcagcat
attctgatgaccagcgcgccgaacaccattaccgtgctggaactggaatgggcgccgtgcagcagcgatgatccgg
aactgtgcgcggtgacctttgtggatcgcccggtgattggcagcagcgtgacctatgatcatccgagcaaaccggt
ggaaaaacgcctgatgccgccgccgccgcagaaaaacaaagatagctggctggatcatgtg;
or
(SEQ ID NO: 1034)
atgcccggctttctcgtgcggattctcctgctgctgctggtgcttctgctgctgggccctaccagaggcc
tgagaaacgccacccagcggatgttcgagatcgactacagccgggacagcttcctgaaggacggccagcccttccg
gtacatcagcggcagcatccactacagcagagtgccccggttctactggaaggaccggctgctgaagatgaagatg
gccggcctgaacgccatccagacctacgtgccctggaacttccacgagccttggcctggccagtaccagttcagcg
aggaccacgacgtggaatactttctgcggctggcccacgagctgggcctgctcgtgattctgaggcctggccctta
catctgcgccgagtgggagatgggaggactgcctgcttggctgctggaaaaagagagcatcctgctgcggagcagc
gaccccgattatctggccgccgtggataagtggctgggcgtgctgctgcccaagatgaagcccctgctgtaccaga
acggcggacccgtgatcaccgtgcaggtggaaaacgagtacggcagctacttcgcctgcgacttcgactacctgcg
gttcctgcagaagcggttcagacaccacctgggcgacgacgtggtgctgttcacaacagacggcgcccacaagacc
tttctgaagtgtggcgctctgcagggcctgtacaccaccgtggattttggcaccggcagcaatatcaccgacgcct
ttctgagccagcggaagtgcgagccaaagggccccctgatcaacagcgagttctacaccggctggctggaccactg
gggccagcctcacagcaccatcaagacagaggccgtggccagcagcctgtacgacatcctggctagaggcgccagc
gtgaacctgtacatgtttatcggcggcaccaacttcgcctactggaacggcgccaacagcccttatgccgcccagc
ccaccagctacgactacgatgcccctctgtctgaggccggcgacctgaccgagaagtactttgccctgcggaacat
catccagaaattcgagaaggtgcccgagggccccatcccccctagcacacctaagttcgcctacggcaaagtgacc
ctggaaaagctgaaaaccgtgggagccgccctggacatcctgtgtcctagcggccctatcaagagcctgtaccccc
tgaccttcatccaagtgaagcagcactacggcttcgtgctgtaccggaccaccctgccccaggactgtagcaatcc
tgccccactgagcagccccctgaacggcgtgcacgatagagcctacgtggccgtggatggcatcccacagggggtg
ctggaacggaacaatgtgatcaccctgaacatcaccggcaaggctggcgccaccctggacctgctggtggaaaaca
tgggcagagtgaactacggcgcctacatcaacgacttcaagggcctggtgtccaacctgaccctgagcagcaacat
cctgaccgactggaccatcttcccactggacaccgaggatgccgtgcggagccatctgggaggatggggacacaga
gatagcggccaccacgatgaagcctgggcccacaacagcagcaactacaccctgcctgccttctacatgggcaact
tcagcatccccagcggcatccccgacctgccacaggacacctttatccagttccccggctggacaaagggacaagt
gtggatcaatggcttcaacctgggcagatactggcccgccagaggccctcagctgaccctgtttgtgccccagcac
attctgatgaccagcgcccccaacaccatcaccgtgctggaactggaatgggccccctgcagcagcgacgaccctg
aactgtgtgccgtgaccttcgtggacaggcccgtgatcggcagcagcgtgacctacgaccaccccagcaagcccgt
ggaaaagcggctgatgcctcccccaccccagaagaacaaggactcctggctggatcacgtgtga.

### Molecular Payloads Targeting GLB1

The GLB1 gene, and mutations therein, are implicated in GM1 gangliosidosis, which can affect the CNS, such as by causing neuronal death. Modulation of GLB1 expression and activity (e.g., by promoting the expression of wild-type GLB1 or a functional fragment thereof and/or activity of a wild-type or mutant form of a protein encoded by GLB1 or a functional fragment thereof) therefore in some embodiments can have a therapeutic effect in subjects with a disorder described herein (e.g., GM1 gangliosidosis).

### Oligonucleotides

GLB1 expression and/or activity in some embodiments can be modulated by the use of oligonucleotides targeting GLB1 sequences. For example, oligonucleotides targeting a mutant sequence of GLB1 may in some embodiments suppress expression of a mutant GLB1 protein, and/or increase expression of a wild-type form of a GLB1 protein.

In some embodiments, an oligonucleotide useful for the treatment of GM1 gangliosidosis, e.g., targeting (e.g., directly or indirectly modulating the expression or activity of) GLB1, comprises a region of complementarity to a GLB1 transcript provided in Table 3, e.g., provided by any one of SEQ ID NOs: 841-845.

### Polypeptides

GLB1 expression and/or activity in some embodiments can be modulated by the use of GLB1 polypeptides or polypeptides that can interact with GLB1 (e.g., to modulate its activity). In some embodiments, a polypeptide useful in the treatment of GM1 gangliosidosis is a peptide, a protein, an enzyme, or an antibody that modulates GLB1, e.g., by interacting with GLB1 or a protein encoded by GLB1, or by providing a protein encoded by GLB1 or a functional fragment thereof (e.g., as an enzyme replacement therapy providing galactosidase beta 1).

In some embodiments, examples of polypeptides (e.g., peptides; proteins, such as enzymes; etc.) useful in the treatment of GM1 gangliosidosis are provided in US20230040603A1, published Feb. 9, 2023, entitled “Compositions useful for treating gm1 gangliosidosis”; US20210381004A1, published Dec. 9, 2021, entitled “RAAV Vectors for the Treatment of GM1 and GM2 Gangliosidosis”; US20130090374A1, published Apr. 11, 2013, entitled “Methods for the treatment of tay-sachs disease, sandhoff disease, and gm1-gangliosidosis”; US20210381004, published Dec. 9, 2012, entitled “RAAV Vectors for the Treatment of GM1 and GM2 Gangliosidosis”; KR20190075594A, published Jul. 1, 2019, entitled “Novel drug delivery composition and pharmaceutical composition for treating gm1 gangliosidosis comprising the same”; the entire contents of each of which are herein incorporated by reference.

Certain polypeptides provided in this section may be useful in treating GM1 gangliosidosis by modulating the activity of genes and gene products other than GLB1 genes/gene products, such as other genes/gene products associated with GM1 gangliosidosis.

### Small Molecules

GLB1 expression and/or activity in some embodiments can be modulated by the use of small molecules that can modulate GLB1 (e.g., to modulate its activity, or its expression). In some embodiments, a small molecule useful in the treatment of GM1 gangliosidosis is a small molecule that modulates GLB1, e.g., by interacting with GLB1 or a protein encoded by GLB1, or by stimulating expression of GLB1.

In some embodiments, examples of small molecules useful in the treatment of GM1 gangliosidosis are provided in US20160068580A1, published Mar. 10, 2016, entitled “Method for treatment of gm1 gangliosidosis”; WO2023042177A1, published Mar. 23, 2023, entitled “Enantiomers of 5-((7-chloroisoquinolin-1-yl)amino)-n-(6-methoxy-1,2,3,4-tetrahydronaphthalen-2-yl)picolinamide”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

### Gene Therapies

GLB1 expression and/or activity in some embodiments can be modulated by the use of gene therapies that can modulate GLB1 (e.g., by delivery of nucleic acids encoding GLB1 or other molecules that interact with GBL1). In some embodiments, a gene therapy payload useful in the treatment of GM1 gangliosidosis is a payload that modulates GLB1, e.g., by interacting with GLB1 or a protein encoded by GLB1, by stimulating expression of GLB1 (such as by providing a molecule that encodes galactosidase beta 1 or a functional fragment thereof), or by suppressing expression of GLB1 (such as by providing a molecule that encodes a suppressor of GLB1, such as a mutant form of GLB1).

In some embodiments, examples of gene therapy payloads (e.g., nucleic acids, such as mRNA, cDNA, plasmid DNA, etc.; viral vectors; etc.) useful in the treatment of GM1 gangliosidosis are provided in US20230040603A1, published Feb. 9, 2023, entitled “Compositions useful for treating gm1 gangliosidosis”; US20210381004A1, published Dec. 9, 2021, entitled “RAAV Vectors for the Treatment of GM1 and GM2 Gangliosidosis”; US20130090374A1, published Apr. 11, 2013, entitled “Methods for the treatment of tay-sachs disease, sandhoff disease, and gm1-gangliosidosis”; US20210381004, published Dec. 9, 2012, entitled “RAAV Vectors for the Treatment of GM1 and GM2 Gangliosidosis”; KR20190075594A, published Jul. 1, 2019, entitled “Novel drug delivery composition and pharmaceutical composition for treating gm1 gangliosidosis comprising the same”; the entire contents of each of which are herein incorporated by reference.

Certain gene therapy payloads provided in this section may be useful in treating GM1 gangliosidosis by modulating the activity of genes and gene products other than GLB1 genes/gene products, such as other genes/gene products associated with GM1 gangliosidosis.

### Molecular Payloads for the Treatment of Niemann-Pick Type A

Various molecular payloads may be useful in the treatment of Niemann-Pick Type A disease (NPA), including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of NPA may include, in some embodiments, molecular payloads which modulate (e.g., increase or decrease) expression or activity of ASM. In some embodiments, treatment of NPA comprises enzyme replacement therapy (ERT), comprising delivery to a cell or tissue of a subject in need thereof a functional form of an acid sphingomyelinase or a functional fragment thereof. In some embodiments, treatment of NPA comprises enzyme replacement therapy (ERT), comprising delivery to a cell or tissue of a subject in need thereof a nucleic acid (e.g., mRNA or cDNA) encoding a functional form of an acid sphingomyelinase protein or functional fragment thereof.

Examples of small molecules useful for the treatment of NPA include: caspase inhibitors, acetyle-leucine, tocopherol, tocopheryl quinone derivatives, RGFP966, BRD3308, HDAC3-IN-T247 (T247), AR-42, Belinostat, Givinostat, Dacinostat, M344, Panobinostat, Abexinostat, Pracinostat, Quisinostat, Rocilinostat, Scriptaid, Trichostatin A, Vorinostat,

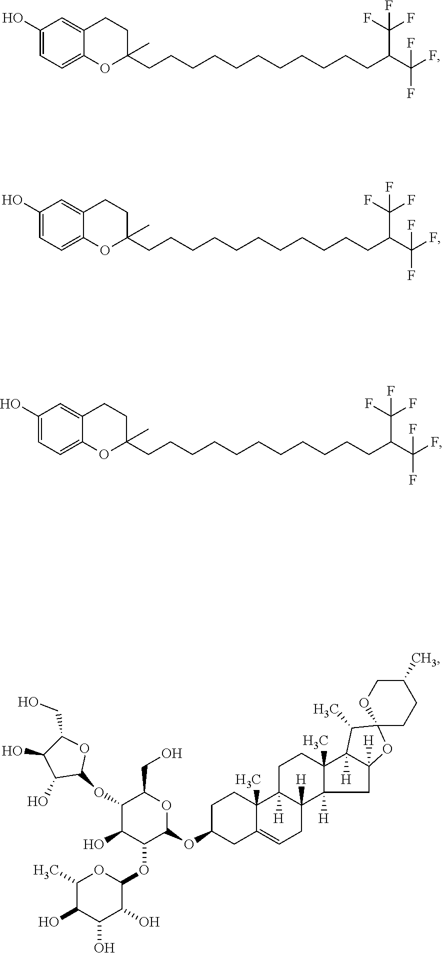

and pharmaceutically acceptable salts, co-crystals, tautomers, stereoisomers, solvates, hydrates, polymorphs, isotopically enriched compounds, and prodrugs thereof.

Examples of polypeptides useful for the treatment of NPA include antibodies, proteins, peptides, and enzymes. In some embodiments, a polypeptide useful for the treatment of NPA comprises an amino acid sequence having at least 70% sequence identity (e.g., at least 70%, 75%, 80%, 85%, 90%, 95%, 96%, 97%, 98%, 99% or 100%) to an amino acid sequence selected from:

(SEQ ID NO: 1035)
MPRYGASLRQSCPRSGREQGQDGTAGAPGLLWMGLVLALALALALALSDS
RVLWAPAEAHPLSPQGHPARLHRIVPRLRDVFGWGNLTCPICKGLFTAIN
LGLKKEPNVARVGSVAIKLCNLLKIAPPAVCQSIVHLFEDDMVEVWRRSV
LSPSEACGLLLGSTCGHWDIFSSWNISLPTVPKPPPKPP SPPAPGAPVS
RILFLTDLHWDHDYLEGTDPDCADPLCCRRGSGLPPASRPGAGYWGEYSK
CDLPLRTLESLLSGLGPAGPFDMVYWTGDIPAHDVWHQTRQDQLRALTTV
TALVRKFLGPVPVYPAVGNHESTPVNSFPPPFIEGNHSSRWLYEAMAKAW
EPWLPAEALRTLRIGGFYALSPYPGLRLISLNMNFCSRENFWLLINSTDP
AGQLQWLVGELQAAEDRGDKVHIIGHIPPGHCLKSWSWNYYRIVARYENT
LAAQFFGHTHVDEFEVFYDEETLSRPLAVAFLAPSATTYIGLNPGYRVYQ
IDGNYSRSSHVVLDHETYILNLTQANIPGAIPHWQLLYRARETYGLPNTL
PTAWHNLVYRMRGDMQLFQTFWFLYHKGHPPSEPCGTPCRLATLCAQLSA
RADSPALCRHLMPDGSLPEAQSLWPRPLFC;
or
(SEQ ID NO: 1036)
MPRYGASLRQSCPRSGREQGQDGTAGAPGLLWMGLALALALALALALSDS
RVLWAPAEAHPLSPQGHPARLHRIVPRLRDVFGWGNLTCPICKGLFTAIN
LGLKKEPNVARVGSVAIKLCNLLKIAPPAVCQSIVHLFEDDMVEVWRRSV
LSPSEACGLLLGSTCGHWDIFSSWNI SLPTVPKPPPKPPSPPAPGAPVS
RILFLTDLHWDHDYLEGTDPDCADPLCCRRGSGLPPASRPGAGYWGEYSK
CDLPLRTLESLLSGLGPAGPFDMVYWTGDIPAHDVWHQTRQDQLRALTTV
TALVRKFLGPVPVYPAVGNHESTPVNSFPPPFIEGNHSSRWLYEAMAKAW
EPWLPAEALRTLRIGGFYALSPYPGLRLISLNMNFCSRENFWLLINSTDP
AGQLQWLVGELQAAEDRGDKVHIIGHIPPGHCLKSWSWNYYRIVARYENT
LAAQFFGHTHVDEFEVFYDEETLSRPLAVAFLAPSATTYIGLNPGYRVYQ
IDGNYSGSSHVVLDHETYILNLTQANIPGAIPHWQLLYRARETYGLPNTL
PTAWHNLVYRMRGDMQLFQTFWFLYHKGHPP SEPCGTPCRLATLCAQLS
ARADSPALCRHLMPDGSLPEAQSLWPRPLFC;
or
(SEQ ID NO: 1037)
MPRYGASLRQSCPRSGREQGQDGTAGAPGLLWMGLVLALALALALALSDS
RVLWAPAEAHPLSPQGHPARLHRIVPRLRDVFGWGNLTCPICKGLFTAIN
LGLKKEPNVARVGSVAIKLCNLLKIAPPAVCQSIVHLFEDDMVEVWRRSV
LSPSEACGLLLGSTCGHWDIFSSWNISLPTVPKPPPKPPSPPAPGAPVSR
ILFLTDLHWDHDYLEGTDPDCADPLCCRRGSGLPPASRPGAGYWGEYSKC
DLPLRTLESLLSGLGPAGPFDMVYWTGDIPAHDVWHQTRQDQLRALTTVT
ALVRKFLGPVPVYPAVGNHESTPVNSFPPPFIEGNHSSRWLYEAMAKAWE
PWLPAEALRTLRIGGFYALSPYPGLRLISLNMNFCSRENFWLLINSTDPA
GQLQWLVGELQAAEDRGDKVHIIGHIPPGHCLKSWSWNYYRIVARYENTL
AAQFFGHTHVDEFEVFYDEETLSRPLAVAFLAPSATTYIGLNPGYRVYQI
DGNYSGSSHVVLDHETYILNLTQANIPGAIPHWQLLYRARETYGLPNTLP
TAWHNLVYRMRGDMQLFQTFWFLYHKGHPPSEPCGTPCRLATLCAQLSAR
ADSPALCRHLMPDGSLPEAQSLWPRPLFC.

Examples of gene therapy payloads useful for the treatment of NPA include: nucleic acids (e.g., mRNA, cDNA, plasmid DNA, etc.), such as those encoding a functional acid sphingomyelinase protein or a functional fragment thereof; viral vectors (e.g., AAV, lentivirus, etc.); etc. In some embodiments, a gene therapy payload useful for the treatment of NPA comprises a nucleic acid comprising a nucleobase sequence having at least 70% sequence identity (e.g., at least 70%, 75%, 80%, 85%, 90%, 95%, 96%, 97%, 98%, 99% or 100%) to a nucleobase sequence selected from:

(SEQ ID NO: 1038)
atgcctagatacggcgcctctctgagacagagctgccctagatctggcagagagcagggccaagacggaa
cagctggtgctcctggactgctgtggatgggacttgctctggctttggctctggccctggctcttgctctgagcga
ttctagagtgctgtgggcccctgccgaagctcatcctttgtctccacaaggacaccccgccagactgcacagaatc
gtgcccagactgagagatgtgttcggctggggcaacctgacctgccctatctgcaagggcctgttcaccgccatca
acctgggcctgaagaaagaacccaacgtggccagagtgggcagcgtggccatcaagctgtgcaacctgctgaagat
tgcccctcctgccgtgtgccagtctatcgtgcacctgttcgaggacgacatggtggaagtgtggcggagaagcgtg
ctgtctccatctgaagcttgtggcctgctgctgggctctacatgtggccactgggacatctttagcagctggaaca
tcagcctgcctaccgtgcctaagcctcctccaaagcctccatctcctccagcacctggcgctccagtgtccagaat
cctgttcctgaccgacctgcactgggaccacgattacctggaaggcaccgatcctgactgcgccgatcctctgtgt
tgcagaagaggctctggactgcctcctgcctctagaccaggtgccggatattggggcgagtacagcaagtgcgacc
tgcctctgagaaccctggaaagcctgctgtctggactgggacctgccggacctttcgatatggtgtactggaccgg
cgacatccccgctcacgacgtgtggcatcagaccagacaggaccagctgagagccctgacaacagtgacagccctc
gtgcggaagtttctgggaccagtgcctgtgtatcccgccgtgggaaatcacgagagcacccctgtgaacagcttcc
ctccacctttcatcgagggcaaccacagcagcaggtggctgtacgaagccatggccaaggcctgggaaccttggct
tccagctgaagcactgcggaccctgagaatcggcggcttttatgccctgtctccttatcctggcctgagactgatc
agcctgaacatgaatttctgcagccgcgagaacttctggctgctgatcaactccacagatcctgccggccagctgc
agtggcttgttggagaattgcaggccgccgaggacagaggcgataaggtgcacatcatcggacacatccctccagg
ccactgcctgaagtcttggagctggaactactaccggattgtggccagatacgagaacaccctggccgctcagttc
ttcggccacacacacgtcgacgagttcgaggtgttctacgacgaggaaaccctgagcagacctctggccgtggcat
ttctggccccaagcgccaccacatatatcggactgaaccccggctaccgggtgtaccagatcgacggcaattacag
cggcagcagccacgtcgtgctggaccacgaaacctacatcctgaatctgacccaggccaacattcccggcgctatc
cctcattggcagctgctgtacagagccagagagacatacggcctgcctaacacactgccaaccgcctggcacaacc
tggtgtacagaatgagaggcgacatgcagctgtttcagaccttctggttcctgtaccacaaggggcaccctccaag
cgagccttgtggcacaccttgtagactggccactctgtgcgctcagctgtccgccagagctgattctcctgctctg
tgcagacacctgatgcctgatggaagcctgcctgaggctcagagcctttggcctagacctctgttctgctgatga;
or
(SEQ ID NO: 1039)
atgcctagatacggcgcctctctgagacagagctgccctagatctggcagagagcagggccaagacggaa
cagctggtgctcctggactgctgtggatgggacttgtcctggctttggctctggccctggctcttgctctgagcga
ttctagagtgctgtgggcccctgccgaagctcatcctttgtctccacaaggacaccccgccagactgcacagaatc
gtgcccagactgagagatgtgttcggctggggcaacctgacctgccctatctgcaagggcctgttcaccgccatca
acctgggcctgaagaaagaacccaacgtggccagagtgggcagcgtggccatcaagctgtgcaacctgctgaagat
tgcccctcctgccgtgtgccagtctatcgtgcacctgttcgaggacgacatggtggaagtgtggcggagaagcgtg
ctgtctccatctgaagcttgtggcctgctgctgggctctacatgtggccactgggacatctttagcagctggaaca
tcagcctgcctaccgtgcctaagcctcctccaaagcctccatctcctccagcacctggcgctccagtgtccagaat
cctgttcctgaccgacctgcactgggaccacgattacctggaaggcaccgatcctgactgcgccgatcctctgtgt
tgcagaagaggctctggactgcctcctgcctctagaccaggtgccggatattggggcgagtacagcaagtgcgacc
tgcctctgagaaccctggaaagcctgctgtctggactgggacctgccggacctttcgatatggtgtactggaccgg
cgacatccccgctcacgacgtgtggcatcagaccagacaggaccagctgagagccctgacaacagtgacagccctc
gtgcggaagtttctgggaccagtgcctgtgtatcccgccgtgggaaatcacgagagcacccctgtgaacagcttcc
ctccacctttcatcgagggcaaccacagcagcaggtggctgtacgaagccatggccaaggcctgggaaccttggct
tccagctgaagcactgcggaccctgagaatcggcggcttttatgccctgtctccttatcctggcctgagactgatc
agcctgaacatgaatttctgcagccgcgagaacttctggctgctgatcaactccacagatcctgccggccagctgc
agtggcttgttggagaattgcaggccgccgaggacagaggcgataaggtgcacatcatcggacacatccctccagg
ccactgcctgaagtcttggagctggaactactaccggattgtggccagatacgagaacaccctggccgctcagttc
ttcggccacacacacgtcgacgagttcgaggtgttctacgacgaggaaaccctgagcagacctctggccgtggcat
ttctggccccaagcgccaccacatatatcggactgaaccccggctaccgggtgtaccagatcgacggcaattacag
cggcagcagccacgtcgtgctggaccacgaaacctacatcctgaatctgacccaggccaacattcccggcgctatc
cctcattggcagctgctgtacagagccagagagacatacggcctgcctaacacactgccaaccgcctggcacaacc
tggtgtacagaatgagaggcgacatgcagctgtttcagaccttctggttcctgtaccacaaggggcaccctccaag
cgagccttgtggcacaccttgtagactggccactctgtgcgctcagctgtccgccagagctgattctcctgctctg
tgcagacacctgatgcctgatggaagcctgcctgaggctcagagcctttggcctagacctctgttctgctgatga;
or
(SEQ ID NO: 1040)
atgcctagatacggcgcctctctgagacagagctgccctagatctggcagagagcagggccaagacggaa
cagctggtgctcctggactgctgtggatgggacttgctctggctttggctctggccctggctcttgctctgagcga
ttctagagtgctgtgggcccctgccgaagctcatcctttgtctccacaaggacaccccgccagactgcacagaatc
gtgcccagactgagagatgtgttcggctggggcaacctgacctgccctatctgcaagggcctgttcaccgccatca
acctgggcctgaagaaagaacccaacgtggccagagtgggcagcgtggccatcaagctgtgcaacctgctgaagat
tgcccctcctgccgtgtgccagtctatcgtgcacctgttcgaggacgacatggtggaagtgtggcggagaagcgtg
ctgtctccatctgaagcttgtggcctgctgctgggctctacatgtggccactgggacatctttagcagctggaaca
tcagcctgcctaccgtgcctaagcctcctccaaagcctccatctcctccagcacctggcgctccagtgtccagaat
cctgttcctgaccgacctgcactgggaccacgattacctggaaggcaccgatcctgactgcgccgatcctctgtgt
tgcagaagaggctctggactgcctcctgcctctagaccaggtgccggatattggggcgagtacagcaagtgcgacc
tgcctctgagaaccctggaaagcctgctgtctggactgggacctgccggacctttcgatatggtgtactggaccgg
cgacatccccgctcacgacgtgtggcatcagaccagacaggaccagctgagagccctgacaacagtgacagccctc
gtgcggaagtttctgggaccagtgcctgtgtatcccgccgtgggaaatcacgagagcacccctgtgaacagcttcc
ctccacctttcatcgagggcaaccacagcagcaggtggctgtacgaagccatggccaaggcctgggaaccttggct
tccagctgaagcactgcggaccctgagaatcggcggcttttatgccctgtctccttatcctggcctgagactgatc
agcctgaacatgaatttctgcagccgcgagaacttctggctgctgatcaactccacagatcctgccggccagctgc
agtggcttgttggagaattgcaggccgccgaggacagaggcgataaggtgcacatcatcggacacatccctccagg
ccactgcctgaagtcttggagctggaactactaccggattgtggccagatacgagaacaccctggccgctcagttc
ttcggccacacacacgtcgacgagttcgaggtgttctacgacgaggaaaccctgagcagacctctggccgtggcat
ttctggccccaagcgccaccacatatatcggactgaaccccggctaccgggtgtaccagatcgacggcaattacag
cggcagcagccacgtcgtgctggaccacgaaacctacatcctgaatctgacccaggccaacattcccggcgctatc
cctcattggcagctgctgtacagagccagagagacatacggcctgcctaacacactgccaaccgcctggcacaacc
tggtgtacagaatgagaggcgacatgcagctgtttcagaccttctggttcctgtaccacaaggggcaccctccaag
cgagccttgtggcacaccttgtagactggccactctgtgcgctcagctgtccgccagagctgattctcctgctctg
tgcagacacctgatgcctgacggaagcctgcctgaggctcagagcctttggcctagacctctgttctgctgataa.

### Molecular Payloads Targeting ASM

The ASM gene, and mutations therein, are implicated in NPA, which can affect the CNS, such as by causing death of CNS cells (e.g., neurons). Modulation of ASM expression and activity (e.g., by promoting the expression of wild-type ASM or a functional fragment thereof and/or activity of a wild-type or mutant form of a protein encoded by ASM or a functional fragment thereof) therefore in some embodiments can have a therapeutic effect in subjects with a disorder described herein (e.g., NPA).

### Oligonucleotides

ASM expression and/or activity in some embodiments can be modulated by the use of oligonucleotides targeting ASM sequences. For example, oligonucleotides targeting a mutant sequence of ASM may in some embodiments suppress expression of a mutant acid sphingomyelinase protein, and/or increase expression of a wild-type form of an acid sphingomyelinase protein.

In some embodiments, an oligonucleotide useful for the treatment of NPA, e.g., targeting (e.g., directly or indirectly modulating the expression or activity of) ASM, comprises a region of complementarity to an ASM transcript provided in Table 4, e.g., provided by any one of SEQ ID NOs: 847-851.

### Polypeptides

ASM expression and/or activity in some embodiments can be modulated by the use of ASM polypeptides or polypeptides that can interact with ASM (e.g., to modulate its activity). In some embodiments, a polypeptide useful in the treatment of NPA is a peptide, a protein, an enzyme, or an antibody that modulates ASM, e.g., by interacting with ASM or a protein encoded by ASM, or by providing a protein encoded by ASM or a functional fragment thereof (e.g., as an enzyme replacement therapy providing acid sphingomyelinase).

In some embodiments, examples of polypeptides (e.g., peptides; proteins, such as enzymes; etc.) useful in the treatment of NPA are provided in WO2022165421A1, published Aug. 4, 2022, entitled “Compositions and methods for treatment of niemann pick type a disease”; WO2015080603A1, published Jun. 4, 2015, entitled “Glycoproteins”; the entire contents of each of which are herein incorporated by reference.

Certain polypeptides provided in this section may be useful in treating NPA by modulating the activity of genes and gene products other than ASM genes/gene products, such as other genes/gene products associated with NPA.

### Small Molecules

ASM expression and/or activity in some embodiments can be modulated by the use of small molecules that can modulate ASM (e.g., to modulate its activity, or its expression). In some embodiments, a small molecule useful in the treatment of NPA is a small molecule that modulates ASM, e.g., by interacting with ASM or a protein encoded by ASM, or by stimulating expression of ASM.

In some embodiments, examples of small molecules useful in the treatment of NPA are provided in US20190201359A1, published Jul. 4, 2019, entitled “Pharmaceutical compositions and uses directed to lysosomal storage disorders”; US20160207902A1, published Jul. 21, 2016, entitled “Tocopherol and tocopheryl quinone derivatives as correctors of lysosomal storage disorders”; US20130123214A1, published May 16, 2013, entitled “Use of delta tocopherol for the treatment of lysosomal storage disorders”; CN113893259A, published Jan. 7, 2022, entitled “Application of paris polyphylla saponin 1 in preparation of SMPD1 protein inhibitor”; US20170281635A1, published Oct. 5, 2017, entitled “Fused heterocyclic organic compounds and uses thereof”; WO2023034440A1, published Mar. 9, 2023, entitled “Treatment of neurodegenerative diseases with hdac inhibitors”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

### Gene Therapies

ASM expression and/or activity in some embodiments can be modulated by the use of gene therapies that can modulate ASM (e.g., by delivery of nucleic acids encoding ASM or other molecules that interact with ASM). In some embodiments, a gene therapy payload useful in the treatment of NPA is a payload that modulates ASM, e.g., by interacting with ASM or a protein encoded by ASM, by stimulating expression of ASM (such as by providing a molecule that encodes acid sphingomyelinase or a functional fragment thereof), or by suppressing expression of ASM (such as by providing a molecule that encodes a suppressor of ASM, such as a mutant form of ASM).

In some embodiments, examples of gene therapy payloads (e.g., nucleic acids, such as mRNA, cDNA, plasmid DNA, etc.; viral vectors; etc.) useful in the treatment of NPA are provided in WO2022165421A1, published Aug. 4, 2022, entitled “Compositions and methods for treatment of niemann pick type a disease”; the entire contents of which are herein incorporated by reference.

Certain gene therapy payloads provided in this section may be useful in treating GLB1 by modulating the activity of genes and gene products other than GLB1 genes/gene products, such as other genes/gene products associated with GM1 gangliosidosis.

### Molecular Payloads for the Treatment of Metachromatic Leukodystrophy (MLD)

Various molecular payloads may be useful in the treatment of MLD, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of MLD may include, in some embodiments, molecular payloads which modulate (e.g., increase or decrease) expression or activity of ARSA. Modulation of ARSA expression and activity (e.g., by increasing the expression and/or activity of an ARSA protein, and/or by delivering a functional form of an ARSA protein or fragment thereof to a cell or tissue of a subject) therefore in some embodiments can have a therapeutic effect in subjects with MLD. In some embodiments, gene therapy comprises delivering an ARSA gene or ARSA nucleic acid (e.g., an ARSA mRNA) to a subject. In some embodiments, treatment of MLD comprises enzyme replacement therapy (ERT), comprising delivery to a cell or tissue of a subject in need thereof a functional form of an ARSA protein or a functional fragment thereof. In some embodiments, treatment of MLD comprises enzyme replacement therapy (ERT), comprising delivery to a cell or tissue of a subject in need thereof a nucleic acid (e.g., mRNA or cDNA) encoding a functional form of an ARSA protein or functional fragment thereof.

Examples of small molecules useful for the treatment of MLD include:

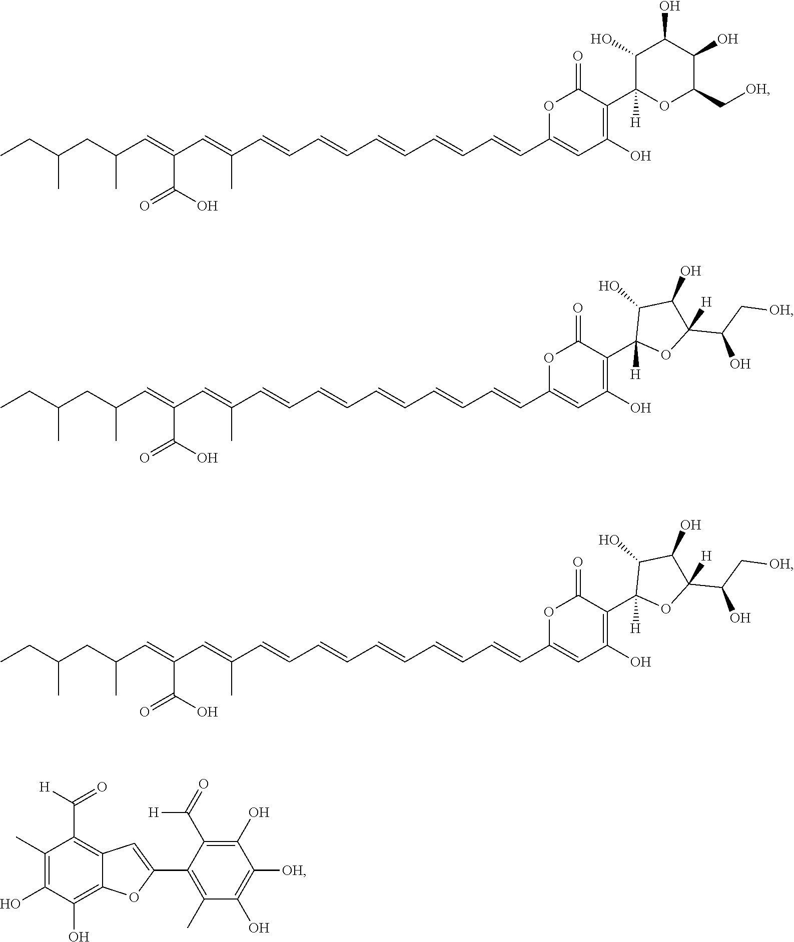 [SMILES: CCC(C)CC(C)/C=C(/C=C(C)/C=C/C=C/C=C/C=C/C=C/c1cc(O)c([C@@H]2O[C@H](CO)[C@H](O)[C@H](O)[C@H]2O)c(=O)o1)C(=O)O ; CCC(C)CC(C)/C=C(/C=C(C)/C=C/C=C/C=C/C=C/C=C/c1cc(O)c([C@H]2O[C@@H]([C@H](O)CO)[C@H](O)[C@H]2O)c(=O)o1)C(=O)O ; CCC(C)CC(C)/C=C(/C=C(C)/C=C/C=C/C=C/C=C/C=C/c1cc(O)c([C@@H]2O[C@@H]([C@H](O)CO)[C@H](O)[C@H]2O)c(=O)o1)C(=O)O ; Cc1c(O)c(O)c(O)c(C=O)c1-c1cc2c(C=O)c(C)c(O)c(O)c2o1]

and pharmaceutically acceptable salts, co-crystals, tautomers, stereoisomers, solvates, hydrates, polymorphs, isotopically enriched compounds, and prodrugs thereof.

Examples of polypeptides useful for the treatment of MLD include antibodies, proteins, peptides, and enzymes. In some embodiments, a polypeptide useful for the treatment of MLD comprises an amino acid sequence having at least 70% sequence identity (e.g., at least 70%, 75%, 80%, 85%, 90%, 95%, 96%, 97%, 98%, 99% or 100%) to an amino acid sequence selected from:

(SEQ ID NO: 1041)
MGAPRSLLLALAAGLAVARPPNIVLIFADDLGYGDLGCYGHPSSTTPNL
DQLAAGGLRFTDFYVPVSLCTPSRAALLTGRLPVRMGMYPGVLVPSSRG
GLPLEEVTVAEVLAARGYLTGMAGKWHLGVGPEGAFLPPHQGFHRFLGI
PYSHDQGPCQNLTCFPPATPCDGGCDQGLVPIPLLANLSVEAQPPWLPG
LEARYMAFAHDLMADAQRQDRPFFLYYASHHTHYPQFSGQSFAERSGRG
PFGDSLMELDAAVGTLMTAIGDLGLLEETLVIFTADNGPETMRMSRGGC
SGLLRCGKGTTYEGGVREPALAFWPGHIAPGVTHELASSLDLLPTLAAL
AGAPLPNVTLDGFDLSPLLLGTGKSPRQSLFFYPSYPDEVRGVFAVRTG
KYKAHFFTQGSAHSDTTADPACHASSSLTAHEPPLLYDLSKDPGENYNL
LGGVAGATPEVLQALKQLQLLKAQLDAAVTFGPSQVARGEDPALQICCH
PGCTPRPACCHCPDPHA;
or
(SEQ ID NO: 1042)
MSMGAPRSLLLALAAGLAVARPPNIVLIFADDLGYGDLGCYGHPSSTTP
NLDQLAAGGLRFTDFYVPVSLCTPSRAALLTGRLPVRMGMYPGVLVPSS
RGGLPLEEVTVAEVLAARGYLTGMAGKWHLGVGPEGAFLPPHQGFHRFL
GIPYSHDQGPCQNLTCFPPATPCDGGCDQGLVPIPLLANLSVEAQPPWL
PGLEARYMAFAHDLMADAQRQDRPFFLYYASHHTHYPQFSGQSFAERSG
RGPFGDSLMELDAAVGTLMTAIGDLGLLEETLVIFTADNGPETMRMSRG
GCSGLLRCGKGTTYEGGVREPALAFWPGHIAPGVTHELASSLDLLPTLA
ALAGAPLPNVTLDGFDLSPLLLGTGKSPRQSLFFYPSYPDEVRGVFAVR
TGKYKAHFFTQGSAHSDTTADPACHASSSLTAHEPPLLYDLSKDPGENY
NLLGGVAGATPEVLQALKQLQLLKAQLDAAVTFGPSQVARGEDPALQIC
CHPGCTPRPACCHCPDPHA;
or
(SEQ ID NO: 1043)
RPPNIVLIFADDLGYGDLGCYGHPSSTTPNLDQLAAGGLRFTDFYVPVS
LCTPSRAALLTGRLPVRMGMYPGVLVPSSRGGLPLEEVTVAEVLAARGY
LTGMAGKWHLGVGPEGAFLPPHQGFHRFLGIPYSHDQGPCQNLTCFPPA
TPCDGGCDQGLVPIPLLANLSVEAQPPWLPGLEARYMAFAHDLMADAQR
QDRPFFLYYASHHTHYPQFSGQSFAERSGRGPFGDSLMELDAAVGTLMT
AIGDLGLLEETLVIFTADNGPETMRMSRGGCSGLLRCGKGTTYEGGVRE
PALAFWPGHIAPGVTHELASSLDLLPTLAALAGAPLPNVTLDGFDLSPL
LLGTGKSPRQSLFFYPSYPDEVRGVFAVRTGKYKAHFFTQGSAHSDTTA
DPACHASSSLTAHEPPLLYDLSKDPGENYNLLGGVAGATPEVLQALKQL
QLLKAQLDAAVTFGPSQVARGEDPALQICCHPGCTPRPACCHCPDPHA.

Examples of gene therapy payloads useful for the treatment of MLD include: nucleic acids (e.g., mRNA, cDNA, plasmid DNA, etc.), such as those encoding a functional ARSA protein or a functional fragment thereof; viral vectors (e.g., AAV, lentivirus, etc.); etc. In some embodiments, a gene therapy payload useful for the treatment of MLD comprises a nucleic acid comprising a nucleobase sequence having at least 70% sequence identity (e.g., at least 70%, 75%, 80%, 85%, 90%, 95%, 96%, 97%, 98%, 99% or 100%) to a nucleobase sequence selected from:

SEQ ID NO: 1044)
atgggagcccctagatctctgctgctggctctggctgctggactggcagttgccagacctcctaacatcg
tgctgatcttcgccgacgatctcggctacggcgatctgggctgttacggacaccccagcagcaccacacctaacct
ggatcaacttgccgctggcggcctgagattcaccgatttctacgtgcccgtgtctctgtgcaccccttctagagct
gctctgctgacaggcagactccctgtgcggatgggaatgtatcctggcgtgctggtgcctagctctagaggcggac
tgcctctggaagaagtgacagttgccgaagtgctggccgccagaggatatctgactggcatggccggaaagtggca
cctcggagttggaccagaaggcgcttttctgcctcctcaccagggcttccaccggtttctgggcatcccttactct
cacgatcagggcccctgccagaacctgacctgttttcctcctgccacaccttgcgacggcggctgtgatcaaggac
tggtgccaattcctctgctggccaacctgagcgtggaagctcaacctccttggctgccaggactggaagcccggta
tatggccttcgctcacgacctgatggccgacgctcagagacaggacagaccattcttcctgtactacgccagccac
cacacacactaccctcagtttagcggccagagcttcgccgagagatctggcagaggacctttcggcgacagcctga
tggaactggatgccgctgtgggcacactgatgacagccatcggagatctgggactgctggaagagacactggtcat
cttcaccgccgacaacggccccgagacaatgagaatgagcagaggcggctgtagcggcctgctgagatgtggcaag
ggcaccacatatgaaggcggcgtgagagaacctgctctggccttttggcctggccatattgctccaggcgtgacac
acgagctggcctcttctctggatctgctgcctacactggcagctcttgctggtgctcccctgcctaatgtgaccct
ggatggcttcgatctgagcccactgctgctcggcacaggcaagtctccaagacagagcctgttcttctaccctagc
taccccgacgaagtgcggggagtgtttgccgtgcggaccggaaagtataaggcccacttcttcacccaaggcagcg
cccactctgacaccacagctgatcctgcttgtcacgccagctctagcctgacagcccatgaacctccactgctgta
cgacctgagcaaggaccccggcgagaactacaatctgcttggcggagttgccggcgctacacctgaagttctgcag
gccctgaaacagctccagctgctgaaagcccagctggacgctgccgtgacatttggacctagtcaggtggccagag
gcgaggatcctgctctgcagatctgttgtcaccctggctgcacacccagacctgcctgctgtcattgtcctgatcc
acacgcc;
(SEQ ID NO: 1045)
atgtctatgggagcccctagatctctgctgctggctctggctgctggactggcagttgccagacctccta
acatcgtgctgatcttcgccgacgatctcggctacggcgatctgggctgttacggacaccccagcagcaccacacc
taacctggatcaacttgccgctggcggcctgagattcaccgatttctacgtgcccgtgtctctgtgcaccccttct
agagctgctctgctgacaggcagactccctgtgcggatgggaatgtatcctggcgtgctggtgcctagctctagag
gcggactgcctctggaagaagtgacagttgccgaagtgctggccgccagaggatatctgactggcatggccggaaa
gtggcacctcggagttggaccagaaggcgcttttctgcctcctcaccagggcttccaccggtttctgggcatccct
tactctcacgatcagggcccctgccagaacctgacctgttttcctcctgccacaccttgcgacggcggctgtgatc
aaggactggtgccaattcctctgctggccaacctgagcgtggaagctcaacctccttggctgccaggactggaagc
ccggtatatggccttcgctcacgacctgatggccgacgctcagagacaggacagaccattcttcctgtactacgcc
agccaccacacacactaccctcagtttagcggccagagcttcgccgagagatctggcagaggacctttcggcgaca
gcctgatggaactggatgccgctgtgggcacactgatgacagccatcggagatctgggactgctggaagagacact
ggtcatcttcaccgccgacaacggccccgagacaatgagaatgagcagaggcggctgtagcggcctgctgagatgt
ggcaagggcaccacatatgaaggcggcgtgagagaacctgctctggccttttggcctggccatattgctccaggcg
tgacacacgagctggcctcttctctggatctgctgcctacactggcagctcttgctggtgctcccctgcctaatgt
gaccctggatggcttcgatctgagcccactgctgctcggcacaggcaagtctccaagacagagcctgttcttctac
cctagctaccccgacgaagtgcggggagtgtttgccgtgcggaccggaaagtataaggcccacttcttcacccaag
gcagcgcccactctgacaccacagctgatcctgcttgtcacgccagctctagcctgacagcccatgaacctccact
gctgtacgacctgagcaaggaccccggcgagaactacaatctgcttggcggagttgccggcgctacacctgaagtt
ctgcaggccctgaaacagctccagctgctgaaagcccagctggacgctgccgtgacatttggacctagtcaggtgg
ccagaggcgaggatcctgctctgcagatctgttgtcaccctggctgcacacccagacctgcctgctgtcattgtcc
tgatccacacgcc;
or
(SEQ ID NO: 1046)
atgtccatgggcgccccccggtctctgctgctggcactggcagcaggactggcagtggccagacccccta
acatcgtgctgatcttcgcagacgatctgggatacggcgacctgggctgctatggccacccaagctccaccacacc
caatctggaccagctggcagcaggaggactgaggttcaccgatttttacgtgccagtgagcctgtgtaccccatcc
agggcagcactgctgacaggcaggctgccagtgcgcatgggcatgtatcctggcgtgctggtgccatctagcaggg
gaggactgccactggaggaggtgaccgtggcagaggtgctggcagccagaggctacctgacaggaatggccggcaa
gtggcacctgggagtgggacctgagggcgccttcctgccaccccaccagggcttccaccggtttctgggcatccct
tattcccacgaccagggcccatgccagaacctgacctgttttcctccagcaacaccatgcgacggaggatgtgatc
agggactggtgcctatcccactgctggccaatctgtctgtggaggcacagccaccttggctgcctggactggaggc
aaggtacatggcattcgcacacgacctgatggcagatgcacagcggcaggatagacctttctttctgtactatgcc
tcccaccacacccactatccacagttcagcggccagtcctttgcagagaggagcggaaggggaccattcggcgact
ccctgatggagctggatgccgccgtgggcaccctgatgacagcaatcggcgacctgggactgctggaggagaccct
ggtcatcttcaccgccgataacggccccgagacaatgcggatgtctagaggaggatgcagcggactgctgagatgt
ggcaagggaaccacatacgagggaggcgtgcgcgagcctgcactggcattttggccaggacacatcgcacctggag
tgacccacgagctggcatcctctctggacctgctgccaacactggcagcactggcaggagcaccactgcctaatgt
gaccctggacggcttcgatctgtctccactgctgctgggcaccggcaagtcccccagacagtctctgttcttttac
cccagctatcctgatgaggtgcggggcgtgtttgccgtgagaaccggcaagtacaaggcccacttctttacacagg
gctctgcccacagcgacaccacagccgatcctgcatgccacgcaagctcctctctgaccgcacacgagccaccact
gctgtacgacctgtccaaggaccccggcgagaactataatctgctgggaggagtggcaggagcaacccctgaggtg
ctgcaggccctgaagcagctgcagctgctgaaggcacagctggacgcagcagtgacattcggaccaagccaggtgg
caaggggagaggaccccgcactgcagatctgctgtcaccctggatgcaccccaaggccagcatgctgtcactgtcc
tgatccacacgcctga;
or
(SEQ ID NO: 1047)
atgtccatgggggcaccgcggtccctcctcctggccctggctgctggcctggccgttgcccgtccgccca
acatcgtgctgatctttgccgacgacctcggctatggggacctgggctgctatgggcaccccagctctaccactcc
caacctggaccagctggcggcgggagggctgcggttcacagacttctacgtgcctgtgtctctgtgcacaccctct
agggccgccctcctgaccggccggctcccggttcggatgggcatgtaccctggcgtcctggtgcccagctcccggg
ggggcctgcccctggaggaggtgaccgtggccgaagtcctggctgcccgaggctacctcacaggaatggccggcaa
gtggcaccttggggtggggcctgagggggccttcctgcccccccatcagggcttccatcgatttctaggcatcccg
tactcccacgaccagggcccctgccagaacctgacctgcttcccgccggccactccttgcgacggtggctgtgacc
agggcctggtccccatcccactgttggccaacctgtccgtggaggcgcagcccccctggctgcccggactagaggc
ccgctacatggctttcgcccatgacctcatggccgacgcccagcgccaggatcgccccttcttcctgtactatgcc
tctcaccacacccactaccctcagttcagtgggcagagctttgcagagcgttcaggccgcgggccatttggggact
ccctgatggagctggatgcagctgtggggaccctgatgacagccataggggacctggggctgcttgaagagacgct
ggtcatcttcactgcagacaatggacctgagaccatgcgtatgtcccgaggcggctgctccggtctcttgcggtgt
ggaaagggaacgacctacgagggcggtgtccgagagcctgccttggccttctggccaggtcatatcgctcccggcg
tgacccacgagctggccagctccctggacctgctgcctaccctggcagccctggctggggccccactgcccaatgt
caccttggatggctttgacctcagccccctgctgctgggcacaggcaagagccctcggcagtctctcttcttctac
ccgtcctacccagacgaggtccgtggggtttttgctgtgcggactggaaagtacaaggctcacttcttcacccagg
gctctgcccacagtgataccactgcagaccctgcctgccacgcctccagctctctgactgctcatgagcccccgct
gctctatgacctgtccaaggaccctggtgagaactacaacctgctggggggtgtggccggggccaccccagaggtg
ctgcaagccctgaaacagcttcagctgctcaaggcccagttagacgcagctgtgaccttcggccccagccaggtgg
cccggggcgaggaccccgccctgcagatctgctgtcatcctggctgcaccccccgcccagcttgctgccattgccc
agatccccatgcctga;
or
(SEQ ID NO: 1048)
atgtccatgggtgcccctagaagtttgctgcttgcattggctgctggcctggctgtggcaagacctccta
atattgtgttgatttttgccgatgatctgggttacggagatctgggttgctacggccaccctagtagcacgactcc
caatctggaccagttggctgcagggggccttaggttcaccgatttctatgtcccagtgtccctgtgtactccttca
cgggcagcattgctgacaggtagattgccggttaggatgggcatgtacccaggagtgctggttccttctagcaggg
ggggactgccactggaagaggtgactgtggctgaggttctggcagctagaggctacctgacaggaatggcaggcaa
gtggcatcttggagtcggtcccgaaggggcctttctcccaccacaccagggcttccaccgcttcctgggtattcct
tactctcacgatcagggaccatgccagaatctgacctgctttcctcctgcgactccctgcgatggcggttgtgacc
agggacttgttcctatcccactcttggctaatctttctgttgaggctcagccaccatggttgcccgggcttgaagc
gaggtacatggcgttcgcacatgaccttatggcagacgctcagaggcaagaccggccttttttcctctattacgcc
agccaccatacccactatccacagttcagtggacagtcttttgcagaaagatctggtagagggccatttggggact
ccctgatggagctggacgctgctgtgggtaccctgatgaccgcgatcggggatttggggctgttggaggagacatt
ggtgatattcaccgctgataacggtcctgaaacgatgagaatgtctagagggggatgttctggactcttgaggtgt
gggaagggcaccacatacgaggggggagttagagaacccgcccttgcgttttggcctggacacatcgccccaggtg
tcacgcatgagctcgcatcctctcttgacctgctgcctacccttgcagctctggctggagcacctctcccaaacgt
gaccctggatggcttcgacctctccccactgttgctgggaacaggcaaatccccccgacagtcactgttcttctac
cctagctatcctgacgaagtcagaggagtgtttgcagtccgcactggtaaatacaaagctcactttttcactcagg
gatccgctcattccgataccactgctgaccccgcttgccacgcttcaagcagtttgacagcccacgaacccccact
cctgtacgacctgagcaaggatccgggcgagaattataatctcctgggtggagttgctggcgctaccccagaagtg
ctgcaggctctcaagcagctgcagttgctgaaggcccagctggacgcagccgtgacatttggtccatcccaggtgg
caagaggcgaagacccagccctgcaaatctgttgtcatcctggatgtacccctcggccagcctgctgtcattgtcc
agatccccacgcctga.

### Molecular Payloads Targeting ARSA

The ARSA gene, and mutations therein, are implicated in MLD. Modulation of ARSA expression and activity (e.g., by promoting the expression of wild-type ARSA or a functional fragment thereof and/or activity of a wild-type or mutant form of a protein encoded by ARSA or a functional fragment thereof) therefore in some embodiments can have a therapeutic effect in subjects with a disorder described herein (e.g., MLD).

### Oligonucleotides

ARSA expression and/or activity in some embodiments can be modulated by the use of oligonucleotides targeting ARSA sequences. For example, oligonucleotides targeting a mutant sequence of ARSA may in some embodiments suppress expression of a mutant ARSA protein, and/or increase expression of a wild-type form of an ARSA protein.

In some embodiments, an oligonucleotide useful for the treatment of metachromatic leukodystrophy, e.g., targeting (e.g., directly or indirectly modulating the expression or activity of) ARSA, comprises a region of complementarity to an ARSA transcript provided in Table 4, e.g., provided by any one of SEQ ID NOs: 852-860.

### Polypeptides

ARSA expression and/or activity in some embodiments can be modulated by the use of ARSA polypeptides or polypeptides that can interact with ARSA (e.g., to modulate its activity). In some embodiments, a polypeptide useful in the treatment of MLD is a peptide, a protein, an enzyme, or an antibody that modulates ARSA, e.g., by interacting with ARSA or a protein encoded by ARSA, or by providing a protein encoded by ARSA or a functional fragment thereof (e.g., as an enzyme replacement therapy).

In some embodiments, examples of polypeptides (e.g., peptides; proteins, such as enzymes; etc.) useful in the treatment of MLD are provided in US20080003211A1, published Jan. 3, 2008, entitled “Production and Purification of Recombinant Arylsulftase”; US20220228170A1, published Jul. 21, 2022, entitled “Compositions useful in treatment of metachromatic leukodystrophy”; WO2023081750A1, published May 11, 2023, entitled “Compositions and methods for treating metachromatic leukodystrophy disease and related disorders”; WO2022255906A1, published Dec. 8, 2022, entitled “Drug for genetic and gene-cell-based therapy”; CN115838765A, published May 24, 2023, entitled “Lentiviral vector for treating metachromatic leukodystrophy”; and US20200179492A1, published Jun. 1, 2021, entitled “Methods and compositions for cns delivery of arylsulfatase a”; the entire contents of each of which are herein incorporated by reference.

Certain polypeptides provided in this section may be useful in treating MLD by modulating the activity of genes and gene products other than ARSA genes/gene products, such as other genes/gene products associated with MLD.

### Small Molecules

ARSA expression and/or activity in some embodiments can be modulated by the use of small molecules that can modulate ARSA (e.g., to modulate its activity, or its expression). In some embodiments, a small molecule useful in the treatment of MLD is a small molecule that modulates ARSA, e.g., by interacting with ARSA or a protein encoded by ARSA, or by stimulating expression of ARSA.

In some embodiments, examples of small molecules useful in the treatment of MLD are provided in WO2018149864A1, published Aug. 23, 2018, entitled “Small molecule therapeutics for treating metachromatic leukodystrophy”; the entire contents of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

### Gene Therapies

ARSA expression and/or activity in some embodiments can be modulated by the use of gene therapies that can modulate ARSA (e.g., by delivery of nucleic acids encoding ARSA or other molecules that interact with ARSA). In some embodiments, a gene therapy payload useful in the treatment of MLD is a payload that modulates ARSA, e.g., by interacting with ARSA or a protein encoded by ARSA, by stimulating expression of ARSA (such as by providing a molecule that encodes ARSA or a functional fragment thereof), or by suppressing expression of ARSA (such as by providing a molecule that encodes a suppressor of ARSA).

In some embodiments, examples of gene therapy payloads (e.g., nucleic acids, such as mRNA, cDNA, plasmid DNA, etc.; viral vectors; etc.) useful in the treatment of MLD are provided in US20080003211A1, published Jan. 3, 2008, entitled “Production and Purification of Recombinant Arylsulftase”; US20220228170A1, published Jul. 21, 2022, entitled “Compositions useful in treatment of metachromatic leukodystrophy”; WO2023081750A1, published May 11, 2023, entitled “Compositions and methods for treating metachromatic leukodystrophy disease and related disorders”; WO2022255906A1, published Dec. 8, 2022, entitled “Drug for genetic and gene-cell-based therapy”; CN115838765A, published May 24, 2023, entitled “Lentiviral vector for treating metachromatic leukodystrophy”; and US20200179492A1, published Jun. 1, 2021, entitled “Methods and compositions for cns delivery of arylsulfatase a”; the entire contents of each of which are herein incorporated by reference.

Certain gene therapy payloads provided in this section may be useful in treating MLD by modulating the activity of genes and gene products other than ARSA genes/gene products, such as other genes/gene products associated with MLD.

### Molecular Payloads for the Treatment of Krabbe Disease

Various molecular payloads may be useful in the treatment of Krabbe disease, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of Krabbe disease may include, in some embodiments, molecular payloads which modulate (e.g., increase or decrease) expression or activity of GALC. Modulation of GALC expression and activity (e.g., by suppressing the expression and/or activity of mutant GALC protein) therefore in some embodiments can have a therapeutic effect in subjects with Krabbe disease. In some embodiments, gene therapy comprises delivering a GALC gene or GALC nucleic acid (e.g., a GALC mRNA) to a subject. In some embodiments, treatment of Krabbe disease comprises enzyme replacement therapy (ERT), comprising delivery to a cell or tissue of a subject in need thereof a functional form of a galactosylceramidase protein or a functional fragment thereof. In some embodiments, treatment of Krabbe disease comprises enzyme replacement therapy (ERT), comprising delivery to a cell or tissue of a subject in need thereof a nucleic acid (e.g., mRNA or cDNA) encoding a functional form of a galactosylceramidase protein or functional fragment thereof.

Examples of small molecules useful for the treatment of Krabbe disease include:

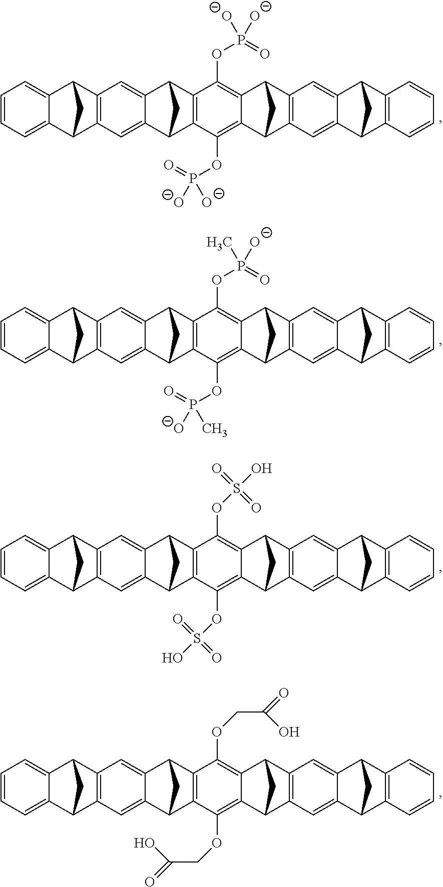

and pharmaceutically acceptable salts, co-crystals, tautomers, stereoisomers, solvates, hydrates, polymorphs, isotopically enriched compounds, and prodrugs thereof.

Examples of polypeptides useful for the treatment of Krabbe disease include antibodies, proteins, peptides, and enzymes. In some embodiments, a polypeptide useful for the treatment of Krabbe disease comprises an amino acid sequence having at least 70% sequence identity (e.g., at least 70%, 75%, 80%, 85%, 90%, 95%, 96%, 97%, 98%, 99% or 100%) to an amino acid sequence selected from:

(SEQ ID NO: 1049)
MAEWLLSASWQRRAKAMTAAAGSAGRAAVPLLLCALLAPGGAYVLDDSD
GLGREFDGIGAVSGGGATSRLLVNYPEPYRSQILDYLFKPNFGASLHIL
KVEIGGDGQTTDGTEPSHMHYALDENYFRGYEWWLMKEAKKRNPNITLI
GLPWSFPGWLGKGFDWPYVNLQLTAYYVVTWIVGAKRYHDLDIDYIGIW
NERSYNANYIKILRKMLNYQGLQRVKIIASDNLWESISASMLLDAELFK
VVDVIGAHYPGTHSAKDAKLTGKKLWSSEDFSTLNSDMGAGCWGRILNQ
NYINGYMTSTIAWNLVASYYEQLPYGRCGLMTAQEPWSGHYVVESPVWV
SAHTTQFTQPGWYYLKTVGHLEKGGSYVALTDGLGNLTIIIETMSHKHS
KCIRPFLPYFNVSQQFATFVLKGSFSEIPELQVWYTKLGKTSERFLFKQ
LDSLWLLDSDGSFTLSLHEDELFTLTTLTTGRKGSYPLPPKSQPFPSTY
KDDFNVDYPFFSEAPNFADQTGVFEYFTNIEDPGEHHFTLRQVLNQRPI
TWAADASNTISIIGDYNWTNLTIKCDVYIETPDTGGVFIAGRVNKGGIL
IRSARGIFFWIFANGSYRVTGDLAGWIIYALGRVEVTAKKWYTLTLTIK
GHFTSGMLNDKSLWTDIPVNFPKNGWAAIGTHSFEFAQFDNFLVEATR;
or
(SEQ ID NO: 1050)
MAEWLLSASWQRRAKAMTAAAGSAGRAAVPLLLCALLAPGGAYVLDDSD
GLGREFDGIGAVSGGGATSRLLVNYPEPYRSQILDYLFKPNFGASLHIL
KVEIGGDGQTTDGTEPSHMHYALDENYFRGYEWWLMKEAKKRNPNITLI
GLPWSFPGWLGKGFDWPYVNLQLTAYYVVTWIVGAKRYHDLDIDYIGIW
NERSYNANYIKILRKMLNYQGLQRVKIIASDNLWESISASMLLDAELFK
VVDVIGAHYPGTHSAKDAKLTGKKLWSSEDESTLNSDMGAGCWGRILNQ
NYINGYMTSTIAWNLVASYYEQLPYGRCGLMTAQEPWSGHYVVESPVWV
SAHTTQFTQPGWYYLKTVGHLEKGGSYVALTDGLGNLIIIIETMSHKHS
KCIRPFLPYFNVSQQFATFVLKGSFSEIPELQVWYTKLGKTSERFLFKQ
LDSLWLLDSDGSFTLSLHEDELFTLTTLTTGRKGSYPLPPKSQPFPSTY
KDDFNVDYPFFSEAPNFADQTGVFEYFTNIEDPGEHHFTLRQVLNQRPI
TWAADASNTISIIGDYNWTNLTIKCDVYIETPDTGGVFIAGRVNKGGIL
IRSARGIFFWIFANGSYRVTGDLAGWIIYALGRVEVTAKKWYTLTLTIK
GHFASGMLNDKSLWTDIPVNFPKNGWAAIGTHSFEFAQFDNFLVEATR;
or
(SEQ ID NO: 1051)
GAYVLDDSDGLGREFDGIGAVSGGGATSRLLVNYPEPYRSQILDYLFKP
NFGASLHILKVEIGGDGQTTDGTEPSHMHYALDENYFRGYEWWLMKEAK
KRNPNITLIGLPWSFPGWLGKGFDWPYVNLQLTAYYVVTWIVGAKRYHD
LDIDYIGIWNERSYNANYIKILRKMLNYQGLQRVKIIASDNLWESISAS
MLLDAELFKVVDVIGAHYPGTHSAKDAKLTGKKLWSSEDFSTLNSDMGA
GCWGRILNQNYINGYMTSTIAWNLVASYYEQLPYGRCGLMTAQEPWSGH
YVVESPVWVSAHTTQFTQPGWYYLKTVGHLEKGGSYVALTDGLGNLTII
IETMSHKHSKCIRPFLPYFNVSQQFATFVLKGSFSEIPELQVWYTKLGK
TSERFLFKQLDSLWLLDSDGSFTLSLHEDELFTLTTLTTGRKGSYPLPP
KSQPFPSTYKDDFNVDYPFFSEAPNFADQTGVFEYFTNIEDPGEHHFTL
RQVLNQRPITWAADASNTISIIGDYNWTNLTIKCDVYIETPDTGGVFIA
GRVNKGGILIRSARGIFFWIFANGSYRVTGDLAGWIIYALGRVEVTAKK
WYTLTLTIKGHFTSGMLNDKSLWTDIPVNFPKNGWAAIGTHSFEFAQFD
NFLVEAT.

Examples of gene therapy payloads useful for the treatment of Krabbe disease include: nucleic acids (e.g., mRNA, cDNA, plasmid DNA, etc.), such as those encoding a functional galactosylceramidase or a functional fragment thereof; viral vectors (e.g., AAV, lentivirus, etc.); etc. In some embodiments, a gene therapy payload useful for the treatment of Krabbe disease comprises a nucleic acid comprising a nucleobase sequence having at least 70% sequence identity (e.g., at least 70%, 75%, 80%, 85%, 90%, 95%, 96%, 97%, 98%, 99% or 100%) to a nucleobase sequence selected from:

(SEQ ID NO: 1052)
atggctgagtggctactctcggcttcctggcaacgccgagcgaaagctatgactgcggccgcgggttcgg
cgggccgcgccgcggtgcccttgctgctgtgtgcgctgctggcgcccggcggcgcgtacgtgctcgacgactccga
cgggctgggccgggagttcgacggcatcggcgcggtcagcggcggcggggcaacctcccgacttctagtaaattac
ccagagccctatcgttctcagatattggattatctctttaagccgaattttggtgcctctttgcatattttaaaag
tggaaataggtggtgatgggcagacaacagacggcactgagccctcccacatgcattatgcactagatgagaatta
tttccgaggatacgagtggtggttgatgaaagaagctaagaagaggaatcccaatattacactcattgggttgcca
tggtcattccctggatggctgggaaaaggtttcgactggccttatgtcaatcttcagctgactgcctattatgtcg
tgacctggattgtgggcgccaagcgttaccatgatttggacattgattatattggaatttggaatgagaggtcata
taatgccaattatattaagatattaagaaaaatgctgaattatcaaggtctccagcgagtgaaaatcatagcaagt
gataatctctgggagtccatctctgcatccatgctccttgatgccgaactcttcaaggtggttgatgttatagggg
ctcattatcctggaacccattcagcaaaagatgcaaagttgactgggaagaagctttggtcttctgaagactttag
cactttaaatagtgacatgggtgcaggctgctggggtcgcattttaaatcagaattatatcaatggctatatgact
tccacaatcgcatggaatttagtggctagttactatgaacagttgccttatgggagatgcgggttgatgacggccc
aggagccatggagtgggcactacgtggtagaatctcctgtctgggtatcagctcataccactcagtttactcaacc
tggctggtattacctgaagacagttggccatttagagaaaggaggaagctacgtagctctgactgatggcttaggg
aacctcaccatcatcattgaaaccatgagtcataaacattctaagtgcatacggccatttcttccttatttcaatg
tgtcacaacaatttgccacctttgttcttaagggatcttttagtgaaataccagagctacaggtatggtataccaa
acttggaaaaacatccgaaagatttctttttaagcagctggattctctatggctccttgacagcgatggcagtttc
acactgagcctgcatgaagatgagctgttcacactcaccactctcaccactggtcgcaaaggcagctacccgcttc
ctccaaaatcccagcccttcccaagtacctataaggatgatttcaatgttgattacccattttttagtgaagctcc
aaactttgctgatcaaactggtgtatttgaatattttacaaatattgaagaccctggcgagcatcacttcacgcta
cgccaagttctcaaccagagacccattacgtgggctgccgatgcatccaacacaatcagtattataggagactaca
actggaccaatctgactataaagtgtgatgtatacatagagacccctgacacaggaggtgtgttcattgcaggaag
agtaaataaaggtggtattttgattagaagtgccagaggaattttcttctggatttttgcaaatggatcttacagg
gttacaggtgatttagctggatggattatatatgctttaggacgtgttgaagttacagcaaaaaaatggtatacac
tcacgttaactattaagggtcatttcacctctggcatgctgaatgacaagtctctgtggacagacatccctgtgaa
ttttccaaagaatggctgggctgcaattggaactcactcctttgaatttgcacagtttgacaactttcttgtggaa
gccacacgctaa;
or
(SEQ ID NO: 1053)
ggcgcgtacgtgctcgacgactccgacgggctgggccgggagttcgacggcatcggcgcggtcagcggcg
gcggggcaacctcccgacttctagtaaattacccagagccctatcgttctcagatattggattatctctttaagcc
gaattttggtgcctctttgcatattttaaaagtggaaataggtggtgatgggcagacaacagacggcactgagccc
tcccacatgcattatgcactagatgagaattatttccgaggatacgagtggtggttgatgaaagaagctaagaaga
ggaatcccaatattacactcattgggttgccatggtcattccctggatggctgggaaaaggtttcgactggcctta
tgtcaatcttcagctgactgcctattatgtcgtgacctggattgtgggcgccaagcgttaccatgatttggacatt
gattatattggaatttggaatgagaggtcatataatgccaattatattaagatattaagaaaaatgctgaattatc
aaggtctccagcgagtgaaaatcatagcaagtgataatctctgggagtccatctctgcatccatgctccttgatgc
cgaactcttcaaggtggttgatgttataggggctcattatcctggaacccattcagcaaaagatgcaaagttgact
gggaagaagctttggtcttctgaagactttagcactttaaatagtgacatgggtgcaggctgctggggtcgcattt
taaatcagaattatatcaatggctatatgacttccacaatcgcatggaatttagtggctagttactatgaacagtt
gccttatgggagatgcgggttgatgacggcccaggagccatggagtgggcactacgtggtagaatctcctgtctgg
gtatcagctcataccactcagtttactcaacctggctggtattacctgaagacagttggccatttagagaaaggag
gaagctacgtagctctgactgatggcttagggaacctcaccatcatcattgaaaccatgagtcataaacattctaa
gtgcatacggccatttcttccttatttcaatgtgtcacaacaatttgccacctttgttcttaagggatcttttagt
gaaataccagagctacaggtatggtataccaaacttggaaaaacatccgaaagatttctttttaagcagctggatt
ctctatggctccttgacagcgatggcagtttcacactgagcctgcatgaagatgagctgttcacactcaccactct
caccactggtcgcaaaggcagctacccgcttcctccaaaatcccagcccttcccaagtacctataaggatgatttc
aatgttgattacccattttttagtgaagctccaaactttgctgatcaaactggtgtatttgaatattttacaaata
ttgaagaccctggcgagcatcacttcacgctacgccaagttctcaaccagagacccattacgtgggctgccgatgc
atccaacacaatcagtattataggagactacaactggaccaatctgactataaagtgtgatgtatacatagagacc
cctgacacaggaggtgtgttcattgcaggaagagtaaataaaggtggtattttgattagaagtgccagaggaattt
tcttctggatttttgcaaatggatcttacagggttacaggtgatttagctggatggattatatatgctttaggacg
tgttgaagttacagcaaaaaaatggtatacactcacgttaactattaagggtcatttcacctctggcatgctgaat
gacaagtctctgtggacagacatccctgtgaattttccaaagaatggctgggctgcaattggaactcactcctttg
aatttgcacagtttgacaactttcttgtggaagccaca;
or
(SEQ ID NO: 1054)
atggccgagtggctgctgtctgctagctggcagagaagggccaaggccatgacagccgccgctggatctg
caggcagagctgctgtgcctctgctgctgtgtgcactgctggcacctggcggagcctacgtgctggatgattctga
cggcctgggcagagagttcgacggcatcggagctgtgtctggcggcggagccacaagcagactgctcgtgaactac
cccgagccctacagaagccagatcctggactacctgttcaagcccaacttcggcgccagcctgcacatcctgaagg
tggaaatcggcggcgacggccagaccaccgatggcacagaacctagccacatgcactacgccctggacgagaacta
cttccggggctacgagtggtggctgatgaaggaagccaagaagcggaaccccaacatcaccctgatcggcctgcct
tggagcttccctggctggctgggcaagggcttcgactggccttacgtgaacctgcagctgaccgcctactacgtcg
tgacctggatcgtgggcgccaagagataccacgacctggacatcgactacatcggcatctggaacgagcggagcta
caacgccaactacatcaagatcctgcggaagatgctgaactaccagggcctgcagagagtgaagatcattgccagc
gacaacctgtgggagagcatcagcgcctccatgctgctggacgccgagctgttcaaggtggtggatgtgatcggcg
cccactaccctggcacacactctgccaaggacgccaagctgaccggcaagaagctgtggtccagcgaggacttcag
caccctgaacagcgacatgggagccggctgctggggcagaatcctgaaccagaattacatcaacggctacatgacc
agcacaatcgcctggaacctggtggccagctactacgagcagctgccctacggcagatgcggcctgatgacagccc
aggaaccttggagcggccactacgtggtggaaagccctgtgtgggtgtccgcccacaccacccagtttacacagcc
cggctggtactacctgaaaaccgtgggccacctggaaaagggcggcagctatgtggccctgacagacggactgggc
aacctgaccatcatcatcgagacaatgtcccacaagcacagcaagtgcatcagaccctttctgccctacttcaacg
tgtcccagcagttcgccaccttcgtgctgaagggcagcttcagcgagatccccgaactgcaagtgtggtacaccaa
gctgggaaagaccagcgagcggttcctgtttaagcagctggacagcctgtggctgctggacagcgacggcagcttt
accctgagcctgcacgaggacgagctgtttacactgaccaccctgaccacaggccggaagggctcttaccccctgc
ctcctaagagccagcccttcccaagcacctacaaggacgacttcaatgtggactacccattctttagcgaggcccc
caatttcgccgaccagaccggcgtgttcgagtacttcaccaacatcgaggaccccggcgagcaccacttcaccctg
agacaggtgctgaaccagcggcccatcacctgggctgccgatgccagcaacaccatcagcatcatcggcgactaca
actggaccaatctgacaatcaagtgcgacgtgtacatcgaaacccccgacaccggcggagtgtttatcgccggcag
agtgaacaagggcgggatcctgatcagaagcgccagaggcatcttcttttggatcttcgccaacggcagctacaga
gtgaccggcgatctggccggctggatcatctacgctctgggcagggtggaagtgaccgccaagaaatggtacaccc
tgacactgactatcaagggccacttcgcctccggcatgctgaacgacaagagcctgtggaccgacatccccgtgaa
cttccccaagaatggctgggccgccatcggcacccacagctttgagttcgcccagttcgacaacttcctggtggaa
gccaccaga.

### Molecular Payloads Targeting GALC

The GALC gene, and mutations therein, are implicated in Krabbe disease. Modulation of GALC expression and activity (e.g., by promoting the expression of functional GALC or a functional fragment thereof and/or activity of a wild-type or mutant form of galactosylceramidase or a functional fragment thereof) therefore in some embodiments can have a therapeutic effect in subjects with a disorder described herein.

### Oligonucleotides

GALC expression and/or activity in some embodiments can be modulated by the use of oligonucleotides targeting GALC sequences. For example, oligonucleotides targeting a mutant sequence of GALC may in some embodiments suppress expression of a mutant (e.g., non-functional or improperly functional) form of a galactosylceramidase protein, and/or increase expression of a wild-type (e.g., properly functional) form of a galactosylceramidase protein.

In some embodiments, an oligonucleotide useful for the treatment of Krabbe disease, e.g., targeting (e.g., directly or indirectly modulating the expression or activity of) GALC, comprises a region of complementarity to a GALC transcript provided in Table 4, e.g., provided by any one of SEQ ID NOs: 861-866.

### Polypeptides

GALC expression and/or activity in some embodiments can be modulated by the use of GALC polypeptides or polypeptides that can interact with GALC (e.g., to modulate its activity). In some embodiments, a polypeptide useful in the treatment of Krabbe disease is a peptide, a protein, an enzyme, or an antibody that modulates GALC, e.g., by interacting with GALC or a protein encoded by GALC, or by providing a protein encoded by GALC or a functional fragment thereof (e.g., as an enzyme replacement therapy).

In some embodiments, examples of polypeptides (e.g., peptides; proteins, such as enzymes; etc.) useful in the treatment of Krabbe disease are provided in US20220118108A1, published Apr. 21, 2022, entitled “Compositions useful in treatment of krabbe disease”; US20160263096A1, published Feb. 6, 2018, entitled “Use of hsp70 as a regulator of enzymatic activity”; and WO2023081750A1, published May 11, 2023, entitled “Compositions and methods for treating metachromatic leukodystrophy disease and related disorders”; the entire contents of each of which are herein incorporated by reference.

Certain polypeptides provided in this section may be useful in treating Krabbe disease by modulating the activity of genes and gene products other than GALC genes/gene products.

### Small Molecules

GALC expression and/or activity in some embodiments can be modulated by the use of small molecules that can modulate GALC (e.g., to modulate its activity, or its expression). In some embodiments, a small molecule useful in the treatment of Krabbe disease is a small molecule that modulates GALC, e.g., by interacting with GALC or a protein encoded by GALC, or by stimulating expression of GALC.

In some embodiments, examples of small molecules useful in the treatment of Krabbe disease are provided in US20210052611A1, published Feb. 25, 2021, entitled “Treatment of lysosomal storage disorders”; the entire contents of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

### Gene Therapies

GALC expression and/or activity in some embodiments can be modulated by the use of gene therapies that can modulate GALC (e.g., by delivery of nucleic acids encoding GALC or other molecules that interact with GALC). In some embodiments, a gene therapy payload useful in the treatment of Krabbe disease is a payload that modulates GALC, e.g., by interacting with GALC or a protein encoded by GALC, by stimulating expression of GALC (such as by providing a molecule that encodes galactosylceramidase or a functional fragment thereof), or by suppressing expression of GALC (such as by providing a molecule that encodes a suppressor of GALC, such as a mutant form of GALC).

In some embodiments, examples of gene therapy payloads (e.g., nucleic acids, such as mRNA, cDNA, plasmid DNA, etc.; viral vectors; etc.) useful in the treatment of Krabbe disease are provided in US20220118108A1, published Apr. 21, 2022, entitled “Compositions useful in treatment of krabbe disease”; US20160263096A1, published Feb. 6, 2018, entitled “Use of hsp70 as a regulator of enzymatic activity”; and WO2023081750A1, published May 11, 2023, entitled “Compositions and methods for treating metachromatic leukodystrophy disease and related disorders”; the entire contents of each of which are herein incorporated by reference.

Certain gene therapy payloads provided in this section may be useful in treating Krabbe disease by modulating the activity of genes and gene products other than GALC genes/gene products.

### Molecular Payloads for the Treatment of Tay-Sachs

Various molecular payloads may be useful in the treatment of Tay-Sachs, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of Tay-Sachs may include, in some embodiments, molecular payloads which modulate (e.g., increase or decrease) expression or activity of HEXA. Molecular payloads useful in the treatment of Tay-Sachs may include, in some embodiments, molecular payloads which modulate the breakdown of GM2 gangliosides. In some embodiments, treatment of Tay-Sachs comprises enzyme replacement therapy (ERT), comprising delivery to a cell or tissue of a subject in need thereof a functional form of a hexosaminidase A protein or a functional fragment thereof. In some embodiments, treatment of Tay-Sachs comprises enzyme replacement therapy (ERT), comprising delivery to a cell or tissue of a subject in need thereof a nucleic acid (e.g., mRNA or cDNA) encoding a functional form of a hexosaminidase A protein or functional fragment thereof.

Examples of small molecules useful for the treatment of Tay-Sachs include: an N-alkyl derivative of 1,5-dideoxy-1,5-imino-D-glucitol in which said alkyl group contains from 2-8 carbon atoms; sirolimus; everolimus; temsirolimus; ridaforolimus; N-dimethylglycinate-rapamycin; 32-deoxo-rapamycin; zotarolimus; acrolimus; pimecrolimus;

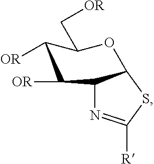

wherein each R is independently selected from H, CO—CH₃, CO—Y, CO—OY and CO—NHY wherein Y is C₁ to C₂₀ alkyl; and R′ is C₁ to C₂₀ alkyl;

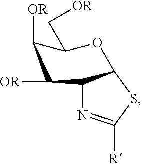

wherein each R is independently selected from H, CO—CH₃, CO—Y, CO—OY and CO—NHY wherein Y is C₁ to C₂₀ alkyl; and R′ is C₁ to C₂₀ alkyl; N-acetylglucosamine-thiazoline or an acetylated derivative thereof, N-acetylgalactosamine-thiazoline or an acetylated derivative thereof; and pharmaceutically acceptable salts, co-crystals, tautomers, stereoisomers, solvates, hydrates, polymorphs, isotopically enriched compounds, and prodrugs thereof.

Examples of polypeptides useful for the treatment of Tay-Sachs include antibodies, proteins, peptides, and enzymes. In some embodiments, a polypeptide useful for the treatment of Tay-Sachs comprises an amino acid sequence having at least 70% sequence identity (e.g., at least 70%, 75%, 80%, 85%, 90%, 95%, 96%, 97%, 98%, 99% or 100%) to the amino acid sequence:

(SEQ ID NO: 1055)
MTSSRLWFSLLLAAAFAGRATALWPWPQNFQTSDQRYVLYPNNFQFQYD
VSSAAQPGCSVLDEAFQRYRDLLFGSGSWPRPYLTGKRHTLEKNVLVVS
VVTPGCNQLPTLESVENYTLTINDDQCLLLSETVWGALRGLETFSQLVW
KSAEGTFFINKTEIEDFPRFPHRGLLLDTSRHYLPLSSILDTLDVMAYN
KLNVFHWHLVDDPSFPYESFTFPELMRKGSYNPVTHIYTAQDVKEVIEY
ARLRGIRVLAEFDTPGHTLSWGPGIPGLLTPCYSGSEPSGTFGPVNPSL
NNTYEFMSTFFLEVSSVFPDFYLHLGGDEVDFTCWKSNPEIQDEMRKKG
FGEDFKQLESFYIQTLLDIVSSYGKGYVVWQEVFDNKVKIQPDTIIQVW
REDIPVNYMKELELVTKAGFRALLSAPWYLNRISYGPDWKDFYIVEPLA
FEGTPEQKALVIGGEACMWGEYVDNTNLVPRLWPRAGAVAERLWSNKLT
SDLTFAYERLSHERCELLRRGVQAQPLNVGFCEQEFEQT.

Examples of gene therapy payloads useful for the treatment of Tay-Sachs include: nucleic acids (e.g., mRNA, cDNA, plasmid DNA, etc.), such as those encoding a functional hexosaminidase A protein or a functional fragment thereof; viral vectors (e.g., AAV, lentivirus, etc.); etc. In some embodiments, a gene therapy payload useful for the treatment of Tay-Sachs comprises a nucleic acid comprising a nucleobase sequence having at least 70% sequence identity (e.g., at least 70%, 75%, 80%, 85%, 90%, 95%, 96%, 97%, 98%, 99% or 100%) to a nucleobase sequence selected from the sequences designated NM_000520.6 or NM_001318825.2.

### Molecular Payloads Targeting HEXA

The HEXA gene, and mutations therein, are implicated in Tay-Sachs, which can affect the CNS, such as by causing neuronal death. Modulation of HEXA expression and activity (e.g., by promoting the expression of wild-type HEXA or a functional fragment thereof and/or activity of a wild-type or mutant form of a protein encoded by HEXA or a functional fragment thereof) therefore in some embodiments can have a therapeutic effect in subjects with a disorder described herein (e.g., Tay-Sachs).

### Oligonucleotides

HEXA expression and/or activity in some embodiments can be modulated by the use of oligonucleotides targeting HEXA sequences. For example, oligonucleotides targeting a mutant sequence of HEXA may in some embodiments suppress expression of a mutant protein encoded by the mutant sequence of HEXA, and/or increase expression of a wild-type form of a protein encoded by HEXA.

In some embodiments, an oligonucleotide useful for the treatment of Tay-Sachs, e.g., targeting (e.g., directly or indirectly modulating the expression or activity of) HEXA, comprises a region of complementarity to a HEXA transcript provided in Table 4, e.g., provided by any one of SEQ ID NOs: 867-868.

### Polypeptides

HEXA expression and/or activity in some embodiments can be modulated by the use of hexosaminidase A polypeptides or polypeptides that can interact with HEXA/hexosaminidase A (e.g., to modulate their activity). In some embodiments, a polypeptide useful in the treatment of Tay-Sachs is a peptide, a protein, an enzyme, or an antibody that modulates HEXA, e.g., by interacting with HEXA or a protein encoded by HEXA, or by providing a protein encoded by HEXA or a functional fragment thereof (e.g., as an enzyme replacement therapy providing hexosaminidase A).

In some embodiments, examples of polypeptides (e.g., peptides; proteins, such as enzymes; etc.) useful in the treatment of Tay-Sachs are provided in US20130090374A1, published Apr. 11, 2013, entitled “Methods for the treatment of tay-sachs disease, sandhoff disease, and gm1-gangliosidosis”; US20210381004, published Dec. 9, 2012, entitled “RAAV Vectors for the Treatment of GM1 and GM2 Gangliosidosis”; U.S. Ser. No. 10/400,226B2, published Sep. 3, 2019, entitled “High functional enzyme having modified substrate specificity of human β-hexosaminidase B and exhibiting protease resistance”; US20210095314A1, published Apr. 1, 2021, entitled “Bicistronic aav vectors encoding hexosaminidase alpha and beta-subunits and uses thereof”; US20040192630A1, published Sep. 30, 2004, entitled “Vectors having both isoforms of beta-hexosaminidase and uses of the same”; WO2022272037A2, published Dec. 29, 2022, entitled “Compositions of beta-hexosaminidase variants and uses thereof”; the entire contents of each of which are herein incorporated by reference.

Certain polypeptides provided in this section may be useful in treating Tay-Sachs by modulating the activity of genes and gene products other than HEXA genes/gene products, such as other genes/gene products associated with Tay-Sachs.

### Small Molecules

HEXA expression and/or activity in some embodiments can be modulated by the use of small molecules that can modulate HEXA (e.g., to modulate its activity, or its expression). In some embodiments, a small molecule useful in the treatment of Tay-Sachs is a small molecule that modulates HEXA, e.g., by interacting with HEXA or a protein encoded by HEXA, or by stimulating expression of HEXA (e.g., a functional form of hexosaminidase A).

In some embodiments, examples of small molecules useful in the treatment of Tay-Sachs are provided in U.S. Pat. No. 5,801,185A, published Sep. 1, 1998, entitled “Method of treating Tay-Sachs disease”; US20070066543A1, published Mar. 22, 2007, entitled “Treatment of tay sachs or sandhoff diseases by enhancing hexosaminidase activity”; US20220142991A1, published May 12, 2022, entitled “Methods and compositions for treatment of lysosomal storage disorder”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

### Gene Therapies

HEXA expression and/or activity in some embodiments can be modulated by the use of gene therapies that can modulate HEXA (e.g., by delivery of nucleic acids encoding HEXA or other molecules that interact with HEXA). In some embodiments, a gene therapy payload useful in the treatment of Tay-Sachs is a payload that modulates HEXA, e.g., by interacting with HEXA or a protein encoded by HEXA, by stimulating expression of HEXA (such as by providing a molecule that encodes hexosaminidase A or a functional fragment thereof), or by suppressing expression of HEXA (such as by providing a molecule that encodes a suppressor of HEXA, such as a mutant form of HEXA).

In some embodiments, examples of gene therapy payloads (e.g., nucleic acids, such as mRNA, cDNA, plasmid DNA, etc.; viral vectors; etc.) useful in the treatment of Tay-Sachs are provided in US20130090374A1, published Apr. 11, 2013, entitled “Methods for the treatment of tay-sachs disease, sandhoff disease, and gm1-gangliosidosis”; US20210381004, published Dec. 9, 2012, entitled “RAAV Vectors for the Treatment of GM1 and GM2 Gangliosidosis”; U.S. Ser. No. 10/400,226B2, published Sep. 3, 2019, entitled “High functional enzyme having modified substrate specificity of human β-hexosaminidase B and exhibiting protease resistance”; US20210095314A1, published Apr. 1, 2021, entitled “Bicistronic aav vectors encoding hexosaminidase alpha and beta-subunits and uses thereof”; US20040192630A1, published Sep. 30, 2004, entitled “Vectors having both isoforms of beta-hexosaminidase and uses of the same”; WO2022272037A2, published Dec. 29, 2022, entitled “Compositions of beta-hexosaminidase variants and uses thereof”; the entire contents of each of which are herein incorporated by reference.

Certain gene therapy payloads provided in this section may be useful in treating Tay-Sachs by modulating the activity of genes and gene products other than HEXA genes/gene products, such as other genes/gene products associated with Tay-Sachs.

### Molecular Payloads for the Treatment of Sandhoff Disease

Various molecular payloads may be useful in the treatment of Sandhoff disease, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of Sandhoff disease may include, in some embodiments, molecular payloads which modulate (e.g., increase or decrease) expression or activity of HEXB. Molecular payloads useful in the treatment of Sandhoff disease may include, in some embodiments, molecular payloads which modulate the breakdown of GM2 gangliosides. In some embodiments, treatment of Sandhoff disease comprises enzyme replacement therapy (ERT), comprising delivery to a cell or tissue of a subject in need thereof a functional form of a hexosaminidase B protein or a functional fragment thereof. In some embodiments, treatment of Sandhoff disease comprises enzyme replacement therapy (ERT), comprising delivery to a cell or tissue of a subject in need thereof a nucleic acid (e.g., mRNA or cDNA) encoding a functional form of a hexosaminidase B protein or functional fragment thereof.

Examples of small molecules useful for the treatment of Sandhoff disease include: sirolimus; everolimus; temsirolimus; ridaforolimus; N-dimethylglycinate-rapamycin; 32-deoxo-rapamycin; zotarolimus; acrolimus; pimecrolimus; and pharmaceutically acceptable salts, co-crystals, tautomers, stereoisomers, solvates, hydrates, polymorphs, isotopically enriched compounds, and prodrugs thereof.

Examples of polypeptides useful for the treatment of Sandhoff disease include antibodies, proteins, peptides, and enzymes. In some embodiments, a polypeptide useful for the treatment of Sandhoff disease comprises an amino acid sequence having at least 70% sequence identity (e.g., at least 70%, 75%, 80%, 85%, 90%, 95%, 96%, 97%, 98%, 99% or 100%) to the amino acid sequence:

(SEQ ID NO: 1056)
MELCGLGLPRPPMLLALLLATLLAAMLALLTQVALVVQVAEAARAPSVS
AKPGPALWPLPLSVKMTPNLLHLAPENFYISHSPNSTAGPSCTLLEEAF
RRYHGYIFGFYKWHHEPAEFQAKTQVQQLLVSITLQSECDAFPNISSDE
SYTLLVKEPVAVLKANRVWGALRGLETFSQLVYQDSYGTFTINESTIID
SPRFSHRGILIDTSRHYLPVKIILKTLDAMAFNKFNVLHWHIVDDQSFP
YQSITFPELSNKGSYSLSHVYTPNDVRMVIEYARLRGIRVLPEFDTPGH
TLSWGKGQKDLLTPCYSRQNKLDSFGPINPTLNTTYSFLTTFFKEISEV
FPDQFIHLGGDEVEFKCWESNPKIQDEMRQKGFGTDFKKLESFYIQKVL
DIIATINKGSIVWQEVFDDKAKLAPGTIVEVWKDSAYPEELSRVTASGF
PVILSAPWYLDLISYGQDWRKYYKVEPLDFGGTQKQKQLFIGGEACLWG
EYVDATNLTPRLWPRASAVGERLWSSKDVRDMDDAYDRLTRHRCRMVER
GIAAQPLYAGYCNHENM.

Examples of gene therapy payloads useful for the treatment of Sandhoff disease include: nucleic acids (e.g., mRNA, cDNA, plasmid DNA, etc.), such as those encoding a functional hexosaminidase B protein or a functional fragment thereof; viral vectors (e.g., AAV, lentivirus, etc.); etc. In some embodiments, a gene therapy payload useful for the treatment of Sandhoff disease comprises a nucleic acid comprising a nucleobase sequence having at least 70% sequence identity (e.g., at least 70%, 75%, 80%, 85%, 90%, 95%, 96%, 97%, 98%, 99% or 100%) to a nucleobase sequence selected from the sequences designated NM_000521.4 or NM_001292004.2.

### Molecular Payloads Targeting HEXB

The HEXB gene, and mutations therein, are implicated in Sandhoff disease, which can affect the CNS, such as by causing neuronal death. Modulation of HEXB expression and activity (e.g., by promoting the expression of wild-type HEXB or a functional fragment thereof and/or activity of a wild-type or mutant form of a protein encoded by HEXB or a functional fragment thereof) therefore in some embodiments can have a therapeutic effect in subjects with a disorder described herein (e.g., Sandhoff disease).

### Oligonucleotides

HEXB expression and/or activity in some embodiments can be modulated by the use of oligonucleotides targeting HEXB sequences. For example, oligonucleotides targeting a mutant sequence of HEXB may in some embodiments suppress expression of a mutant protein encoded by the mutant sequence of HEXB, and/or increase expression of a wild-type form of a protein encoded by HEXB.

In some embodiments, an oligonucleotide useful for the treatment of Sandhoff disease, e.g., targeting (e.g., directly or indirectly modulating the expression or activity of) HEXB, comprises a region of complementarity to a HEXB transcript provided in Table 4, e.g., provided by any one of SEQ ID NOs: 869-870.

### Polypeptides

HEXB expression and/or activity in some embodiments can be modulated by the use of hexosaminidase B polypeptides or polypeptides that can interact with HEXB/hexosaminidase B (e.g., to modulate their activity). In some embodiments, a polypeptide useful in the treatment of Sandhoff disease is a peptide, a protein, an enzyme, or an antibody that modulates HEXB, e.g., by interacting with HEXB or a protein encoded by HEXB, or by providing a protein encoded by HEXB or a functional fragment thereof (e.g., as an enzyme replacement therapy providing hexosaminidase B).

In some embodiments, examples of polypeptides (e.g., peptides; proteins, such as enzymes; etc.) useful in the treatment of Sandhoff disease are provided in US20130090374A1, published Apr. 11, 2013, entitled “Methods for the treatment of tay-sachs disease, sandhoff disease, and gm1-gangliosidosis”; US20210381004, published Dec. 9, 2012, entitled “RAAV Vectors for the Treatment of GM1 and GM2 Gangliosidosis”; U.S. Ser. No. 10/400,226B2, published Sep. 3, 2019, entitled “High functional enzyme having modified substrate specificity of human β-hexosaminidase B and exhibiting protease resistance”; US20210095314A1, published Apr. 1, 2021, entitled “Bicistronic aav vectors encoding hexosaminidase alpha and beta-subunits and uses thereof”; US20040192630A1, published Sep. 30, 2004, entitled “Vectors having both isoforms of beta-hexosaminidase and uses of the same”; WO2022272037A2, published Dec. 29, 2022, entitled “Compositions of beta-hexosaminidase variants and uses thereof”; the entire contents of each of which are herein incorporated by reference.

Certain polypeptides provided in this section may be useful in treating Sandhoff disease by modulating the activity of genes and gene products other than HEXB genes/gene products, such as other genes/gene products associated with Sandhoff disease.

### Small Molecules

HEXB expression and/or activity in some embodiments can be modulated by the use of small molecules that can modulate HEXB (e.g., to modulate its activity, or its expression). In some embodiments, a small molecule useful in the treatment of Sandhoff disease is a small molecule that modulates HEXB, e.g., by interacting with HEXB or a protein encoded by HEXB, or by stimulating expression of HEXB (e.g., a functional form of hexosaminidase B).

In some embodiments, examples of small molecules useful in the treatment of Sandhoff disease are provided in US20070066543A1, published Mar. 22, 2007, entitled “Treatment of tay sachs or sandhoff diseases by enhancing hexosaminidase activity”; US20220142991A1, published May 12, 2022, entitled “Methods and compositions for treatment of lysosomal storage disorder”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

### Gene Therapies

HEXB expression and/or activity in some embodiments can be modulated by the use of gene therapies that can modulate HEXB (e.g., by delivery of nucleic acids encoding HEXB or other molecules that interact with HEXB). In some embodiments, a gene therapy payload useful in the treatment of Sandhoff disease is a payload that modulates HEXB, e.g., by interacting with HEXB or a protein encoded by HEXB, by stimulating expression of HEXB (such as by providing a molecule that encodes hexosaminidase B or a functional fragment thereof), or by suppressing expression of HEXB (such as by providing a molecule that encodes a suppressor of HEXB, such as a mutant form of HEXB).

In some embodiments, examples of gene therapy payloads (e.g., nucleic acids, such as mRNA, cDNA, plasmid DNA, etc.; viral vectors; etc.) useful in the treatment of Sandhoff disease are provided in US20130090374A1, published Apr. 11, 2013, entitled “Methods for the treatment of tay-sachs disease, sandhoff disease, and gm1-gangliosidosis”; US20210381004, published Dec. 9, 2012, entitled “RAAV Vectors for the Treatment of GM1 and GM2 Gangliosidosis”; U.S. Ser. No. 10/400,226B2, published Sep. 3, 2019, entitled “High functional enzyme having modified substrate specificity of human β-hexosaminidase B and exhibiting protease resistance”; US20210095314A1, published Apr. 1, 2021, entitled “Bicistronic aav vectors encoding hexosaminidase alpha and beta-subunits and uses thereof”; US20040192630A1, published Sep. 30, 2004, entitled “Vectors having both isoforms of beta-hexosaminidase and uses of the same”; WO2022272037A2, published Dec. 29, 2022, entitled “Compositions of beta-hexosaminidase variants and uses thereof”; the entire contents of each of which are herein incorporated by reference.

Certain gene therapy payloads provided in this section may be useful in treating Sandhoff disease by modulating the activity of genes and gene products other than HEXA genes/gene products, such as other genes/gene products associated with Sandhoff disease.

### Molecular Payloads for the Treatment of Gaucher Disease, Types II and III

Various molecular payloads may be useful in the treatment of Gaucher disease, types II and III (GD), including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of GD may include, in some embodiments, molecular payloads which modulate (e.g., increase or decrease) expression or activity of GBA. Modulation of GBA expression and activity (e.g., by suppressing the expression and/or activity of mutant protein encoded by GBA) therefore in some embodiments can have a therapeutic effect in subjects with GD. In some embodiments, gene therapy comprises delivering a GBA gene or GBA nucleic acid (e.g., a GBA mRNA) to a subject. In some embodiments, treatment of GD comprises enzyme replacement therapy (ERT), comprising delivery to a cell or tissue of a subject in need thereof a functional form of a β-glucocerebrosidase protein or a functional fragment thereof. In some embodiments, treatment of GD comprises enzyme replacement therapy (ERT), comprising delivery to a cell or tissue of a subject in need thereof a nucleic acid (e.g., mRNA or cDNA) encoding a functional form of a β-glucocerebrosidase protein or functional fragment thereof.

Examples of small molecules useful for the treatment of GD include: N-dodecyl-isofagomine, N-butyl N-(3-cyclohexylpropyl)-isofagomine, N-(3-phenylpropyl)-isofagomine, N-[(2E,6Z,10Z)-3,7,11-trimethyldodecatrienyl]-isofagomine, isofagomine, N-dodecyl-deoxynojirimycin, calystegine A3, calystegine B1, calystegine B2, calystegine C1, and pharmaceutically acceptable salts, co-crystals, tautomers, stereoisomers, solvates, hydrates, polymorphs, isotopically enriched compounds, and prodrugs thereof.

Examples of polypeptides useful for the treatment of GD include antibodies, proteins, peptides, and enzymes. In some embodiments, a polypeptide useful for the treatment of GD comprises an amino acid sequence having at least 70% sequence identity (e.g., at least 70%, 75%, 80%, 85%, 90%, 95%, 96%, 97%, 98%, 99% or 100%) to the amino acid sequence:

(SEQ ID NO: 1057)
MEFSSPSREECPKPLSRVSIMAGSLTGLLLLQAVSWASGARPCIPKSFG
YSSVVCVCNATYCDSFDPPTFPALGTFSRYESTRSGRRMELSMGPIQAN
HTGTGLLLTLQPEQKFQKVKGFGGAMTDAAALNILALSPPAQNLLLKSY
FSEEGIGYNIIRVPMASCDFSIRTYTYADTPDDFQLHNFSLPEEDTKLK
IPLIHRALQLAQRPVSLLASPWTSPTWLKTNGAVNGKGSLKGQPGDIYH
QTWARYFVKFLDAYAEHKLQFWAVTAENEPSAGLLSGYPFQCLGFTPEH
QRDFIARDLGPTLANSTHHNVRLLMLDDQRLLLPHWAKVVLTDPEAAKY
VHGIAVHWYLDFLAPAKATLGETHRLFPNTMLFASEACVGSKFWEQSVR
LGSWDRGMQYSHSIITNLLYHVVGWTDWNLALNPEGGPNWVRNFVDSPI
IVDITKDTFYKQPMFYHLGHFSKFIPEGSQRVGLVASQKNDLDAVALMH
PDGSAVVVVLNRSSKDVPLTIKDPAVGFLETISPGYSIHTYLWRRQ.

Examples of gene therapy payloads useful for the treatment of GD include: nucleic acids (e.g., mRNA, cDNA, plasmid DNA, etc.), such as those encoding a functional 0-glucocerebrosidase or a functional fragment thereof; viral vectors (e.g., AAV, lentivirus, etc.); etc. In some embodiments, a gene therapy payload useful for the treatment of GD comprises a nucleic acid comprising a nucleobase sequence having at least 70% sequence identity (e.g., at least 70%, 75%, 80%, 85%, 90%, 95%, 96%, 97%, 98%, 99% or 100%) to a nucleobase sequence selected from the sequences of NM_000157.4, NM_001005741.3, NM_001005742.3, NM_001171811.2, NM_001171812.2, or:

(SEQ ID NO: 1058)
atggaattcagcagccccagcagagaggaatgccccaagcctctgagcc
gggtgtcaatcatggccggatctctgacaggactgctgctgcttcaggc
cgtgtcttgggcttctggcgctagaccttgcatccccaagagcttcggc
tacagcagcgtcgtgtgcgtgtgcaatgccacctactgcgacagcttcg
accctcctacctttcctgctctgggcaccttcagcagatacgagagcac
cagatccggcagacggatggaactgagcatgggacccatccaggccaat
cacacaggcactggcctgctgctgacactgcagcctgagcagaaattcc
agaaagtgaaaggcttcggcggagccatgacagatgccgccgctctgaa
tatcctggctctgtctccaccagctcagaacctgctgctcaagagctac
ttcagcgaggaaggcatcggctacaacatcatcagagtgcccatggcca
gctgcgacttcagcatcaggacctacacctacgccgacacacccgacga
tttccagctgcacaacttcagcctgcctgaagaggacaccaagctgaag
atccctctgatccacagagccctgcagctggcacaaagacccgtgtcac
tgctggcctctccatggacatctcccacctggctgaaaacaaatggcgc
cgtgaatggcaagggcagcctgaaaggccaacctggcgacatctaccac
cagacctgggccagatacttcgtgaagttcctggacgcctatgccgagc
acaagctgcagttttgggccgtgacagccgagaacgaaccttctgctgg
actgctgagcggctacccctttcagtgcctgggctttacacccgagcac
cagcgggactttatcgcccgtgatctgggacccacactggccaatagca
cccaccataatgtgcggctgctgatgctggacgaccagagactgcttct
gccccactgggctaaagtggtgctgacagatcctgaggccgccaaatac
gtgcacggaatcgccgtgcactggtatctggactttctggcccctgcca
aggccacactgggagagacacacagactgttccccaacaccatgctgtt
cgccagcgaagcctgtgtgggcagcaagttttgggaacagagcgtgcgg
ctcggcagctgggatagaggcatgcagtacagccacagcatcatcacca
acctgctgtaccacgtcgtcggctggaccgactggaatctggccctgaa
tcctgaaggcggccctaactgggtccgaaacttcgtggacagccccatc
atcgtggacatcaccaaggacaccttctacaagcagcccatgttctacc
acctgggacacttcagcaagttcatccccgagggctctcagcgcgttgg
actggtggcttcccagaagaacgatctggacgccgtggctctgatgcac
cctgatggatctgctgtggtggtggtcctgaaccgcagcagcaaagatg
tgcccctgaccatcaaggatcccgccgtgggattcctggaaacaatcag
ccctggctactccatccacacctacctgtggcgtagacag.

### Molecular Payloads Targeting GBA

The GBA gene, and mutations therein, are implicated in GD, which can affect the CNS. Modulation of GBA expression and activity (e.g., by suppressing the expression of GBA or a mutant form thereof and/or activity of a wild-type or mutant form of a protein encoded by GBA) therefore in some embodiments can have a therapeutic effect in subjects with a disorder described herein (e.g., GD).

### Oligonucleotides

GBA expression and/or activity in some embodiments can be modulated by the use of oligonucleotides targeting GBA sequences. For example, oligonucleotides targeting a mutant sequence of GBA may in some embodiments suppress expression of a mutant protein encoded by the mutant sequence of GBA, and/or increase expression of a wild-type form of a protein encoded by GBA.

In some embodiments, an oligonucleotide useful for the treatment of GD, e.g., targeting (e.g., directly or indirectly modulating the expression or activity of) GBA, comprises a region of complementarity to a GBA transcript provided in Table 4, e.g., provided by any one of SEQ ID NOs: 871-875.

### Polypeptides

GBA expression and/or activity in some embodiments can be modulated by the use of β-glucocerebrosidase polypeptides or polypeptides that can interact with GBA/0-glucocerebrosidase (e.g., to modulate their activity). In some embodiments, a polypeptide useful in the treatment of GD is a peptide, a protein, an enzyme, or an antibody that modulates GBA, e.g., by interacting with GBA or a protein encoded by GBA, or by providing a protein encoded by GBA or a functional fragment thereof (e.g., as an enzyme replacement therapy providing β-glucocerebrosidase).

In some embodiments, examples of polypeptides (e.g., peptides; proteins, such as enzymes; etc.) useful in the treatment of GD are provided in U.S. Pat. No. 9,623,090B2, published Apr. 18, 2017, entitled “Compositions and methods for treating type III gaucher disease”; US20200338148A1, published Oct. 29, 2020, entitled “Gene therapies for lysosomal disorders”; the entire contents of each of which are herein incorporated by reference.

Certain polypeptides provided in this section may be useful in treating GD by modulating the activity of genes and gene products other than GBA genes/gene products, such as other genes/gene products associated with GD.

### Small Molecules

GBA expression and/or activity in some embodiments can be modulated by the use of small molecules that can modulate GBA (e.g., to modulate its activity, or its expression). In some embodiments, a small molecule useful in the treatment of GD is a small molecule that modulates GBA, e.g., by interacting with GBA or a protein encoded by GBA, or by stimulating expression of GBA (e.g., a functional form of β-glucocerebrosidase).

In some embodiments, examples of small molecules useful in the treatment of GD are provided in U.S. Pat. No. 6,916,829B2, published Jul. 12, 2005, entitled “Method for enhancing mutant enzyme activity in gaucher disease”; the entire contents of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

### Gene Therapies

GBA expression and/or activity in some embodiments can be modulated by the use of gene therapies that can modulate GBA (e.g., by delivery of nucleic acids encoding GBA or other molecules that interact with GBA). In some embodiments, a gene therapy payload useful in the treatment of GD is a payload that modulates GBA, e.g., by interacting with GBA or a protein encoded by GBA, by stimulating expression of GBA (such as by providing a molecule that encodes β-glucocerebrosidase or a functional fragment thereof), or by suppressing expression of GBA (such as by providing a molecule that encodes a suppressor of GBA, such as a mutant form of GBA).

In some embodiments, examples of gene therapy payloads (e.g., nucleic acids, such as mRNA, cDNA, plasmid DNA, etc.; viral vectors; etc.) useful in the treatment of GD are provided in US20200338148A1, published Oct. 29, 2020, entitled “Gene therapies for lysosomal disorders”; the entire contents of which are herein incorporated by reference.

Certain gene therapy payloads provided in this section may be useful in treating GD by modulating the activity of genes and gene products other than GBA genes/gene products, such as other genes/gene products associated with GD.

### Molecular Payloads for the Treatment of Rett Syndrome

Various molecular payloads may be useful in the treatment of Rett syndrome, including oligonucleotides, polypeptides (e.g., peptides, proteins, enzymes, antibodies, etc.), small molecules (e.g., small molecule inhibitors, etc.), and gene therapies (e.g., nucleic acids and/or nucleic acid vectors encoding therapeutic molecules, such as therapeutic proteins). Molecular payloads useful in the treatment of Rett syndrome may include, in some embodiments, molecular payloads which modulate (e.g., increase or decrease) expression or activity of MECP2.

Examples of oligonucleotides useful for the treatment of Rett syndrome, e.g., oligonucleotides targeting (e.g., directly or indirectly modulating the expression or activity of) genes associated with Rett syndrome (e.g., MECP2), include those listed in Table 19 below. Each oligonucleotide provided in Table 19 may have any modification pattern disclosed herein.

TABLE 19
Oligonucleotides for the treatment of Rett syndrome

| SEQ ID | Target | Oligonucleotide | |
|---|---|---|---|
| NO: | gene | Sequence (5′-3′) | Oligonucleotide Structure (5′-3′) |
| 1070 | MECP2 | AACTCTCTCG | es eo meo eo meo ds mds ds mds ds ds ds mds ds |
| | | GTCACGGGCG | mds eo eo es mes e, wherein: e = 2′-MOE sugar |
| | | | moiety, d = 2′-3-D deoxyribosyl sugar moiety, |
| | | | s = phosphorothioate internucleoside linkage, |
| | | | o = phosphodiester internucleoside linkage |
| 1071 | MECP2 | CACACTGACC | mes eo meo eo meo ds ds ds mds mds ds ds ds mds |
| | | TTTCAGGGCT | ds eo eo es mes e, wherein: e = 2′-MOE sugar |
| | | | moiety, d = 2′-3-D deoxyribosyl sugar moiety, |
| | | | s = phosphorothioate internucleoside linkage, |
| | | | o = phosphodiester internucleoside linkage |
| 1072 | MECP2 | GATCACTGGA | es eo eo meo eo mds ds ds ds ds ds mds ds mds ds |
| | | ACACAATGGT | eo eo es es e, wherein: e = 2′-MOE sugar moiety, |
| | | | d = 2′-3-D deoxyribosyl sugar moiety, |
| | | | s = phosphorothioate internucleoside linkage, |
| | | | o = phosphodiester internucleoside linkage |
| 1073 | MECP2 | CGTGCCATGG | mes eo eo eo meo mds ds ds ds ds ds ds ds ds mds |
| | | AAGTCCTTCC | meo eo es mes me, wherein: e = 2′-MOE sugar moiety, |
| | | | d = 2′-3-D deoxyribosyl sugar moiety, |
| | | | s = phosphorothioate internucleoside linkage, |
| | | | o = phosphodiester internucleoside linkage |
| 1074 | MECP2 | GGTTTTTCTC | es eo eo eo eo ds ds mds ds mds mds ds ds ds ds |
| | | CTTTATTATC | eo eo es es me, wherein: e = 2′-MOE sugar moiety, |
| | | | d = 2′-3-D deoxyribosyl sugar moiety, |
| | | | s = phosphorothioate internucleoside linkage, |
| | | | o = phosphodiester internucleoside linkage |

In each sequence listed in Table 19, each T may be optionally and independently replaced with a U.

Examples of small molecules useful for the treatment of Rett syndrome include: calcipressin 1, calcipressin 2, calcipressin 3, tacrolimus, ascomycin, sirolimus, pimecrolimus, cyclosporin A, voclosporine, LxPVc1, LxPVc2, LxPVc3, 6-(3,4-dichloro-phenyl)-4-(N,N-dimethylaminoethylthio)-2-phenyl-pyrimidine, decitabine, and pharmaceutically acceptable salts, co-crystals, tautomers, stereoisomers, solvates, hydrates, polymorphs, isotopically enriched compounds, and prodrugs thereof.

Examples of polypeptides useful for the treatment of Rett syndrome include antibodies, proteins, peptides, and enzymes. In some embodiments, polypeptides useful for the treatment of Rett syndrome include methyl-CpG binding protein 2 and functional fragments thereof.

### Molecular Payloads Targeting MECP2

The MECP2 gene, which encodes methyl-CpG binding protein 2, and mutations therein, are implicated in Rett syndrome. Modulation of MECP2 expression and activity (e.g., by suppressing the expression and/or activity of mutant MECP2 protein and/or its interactions with other proteins, or by providing or increasing expression and/or activity of wild-type MECP2, methyl-CpG binding protein 2, or a functional fragment thereof) therefore in some embodiments can have a therapeutic effect in subjects with Rett syndrome.

### Oligonucleotides

MECP2 (and/or methyl-CpG binding protein 2 encoded by MECP2) expression and/or activity in some embodiments can be modulated by the use of oligonucleotides targeting MECP2 sequences.

In some embodiments, an oligonucleotide useful for the treatment of Rett syndrome, e.g., targeting (e.g., directly or indirectly modulating the expression or activity of) MECP2, comprises a region of complementarity to an MECP2 transcript provided in Table 4, e.g., provided by any one of SEQ ID NOs: 1059-1068.

In some embodiments, examples of oligonucleotides useful for the treatment of Rett syndrome, e.g., targeting (e.g., directly or indirectly modulating the expression or activity of) MECP2, are provided in US20180036335A1, published Feb. 8, 2018, entitled “Compositions for modulating mecp2 expression”; US20150152410A1, published Jun. 4, 2015, entitled “Compositions and methods for modulating mecp2 expression”; US20180044673A1, published Feb. 15, 2018, entitled “Methods for modulating mecp2 expression”; U.S. Ser. No. 11/129,844B2, published Sep. 28, 2021, entitled “Compositions and methods for modulating MECP2 expression”; WO2023049477A2, published Mar. 30, 2023, entitled “Compositions for editing mecp2 transcripts and methods thereof”; the entire contents of each of which are herein incorporated by reference.

Certain oligonucleotides provided in this section may be useful in treating Rett syndrome by modulating the activity of genes and/or gene products other than MECP2 genes/gene products, such as other genes/gene products associated with Rett syndrome.

### Polypeptides

MECP2 expression and/or activity in some embodiments can be modulated by the use of methyl-CpG binding protein 2 polypeptides or polypeptides that can interact with methyl-CpG binding protein 2 (e.g., to modulate its biological activity and/or its interaction with other biomolecules).

In some embodiments, examples of polypeptides (e.g., peptides; proteins, such as enzymes; antibodies; etc.) useful in the treatment of Rett syndrome are provided in US20130316961A1, published Nov. 28, 2013, entitled “Treatment of mecp-2 associated disorders”; the entire contents of which are herein incorporated by reference.

Certain polypeptides provided in this section may be useful in treating Rett syndrome by modulating the activity of genes and gene products other than MECP2 genes/gene products, such as other genes/gene products associated with Rett syndrome.

### Small Molecules

MECP2 expression and/or activity in some embodiments can be modulated by the use of small molecules that can modulate MECP2 (e.g., to modulate its biological activity, its expression, and/or its interaction with other biomolecules).

In some embodiments, examples of small molecules useful in the treatment of Rett syndrome are provided in US20130316961A1, published Nov. 28, 2013, entitled “Treatment of mecp-2 associated disorders”; WO2022031759A1, published Feb. 10, 2022, entitled “Decitabine induced mecp2 expression and uses thereof”; the entire contents of each of which are herein incorporated by reference.

In some embodiments, the small molecule is a pharmaceutically acceptable salt, co-crystal, tautomer, stereoisomer, solvate, hydrate, polymorph, isotopically enriched compound, or prodrug of a small molecule provided herein.

Certain small molecules provided in this section may be useful in treating Rett syndrome by modulating the activity of genes and gene products other than MECP2 genes/gene products, such as other genes/gene products associated with Rett syndrome.

### Gene Therapies

MECP2 expression and/or activity in some embodiments can be modulated by the use of gene therapies that can modulate MECP2 (e.g., by delivery of nucleic acids encoding MECP2 or other molecules that interact with MECP2).

In some embodiments, gene therapies, such as those involving administration of a compounds encoding useful therapeutic agents, useful in the treatment of Rett syndrome are provided in U.S. Pat. No. 9,415,121B2, published Aug. 16, 2016, entitled “Delivery of MECP2 polynucleotide using recombinant AAV9”; WO2018226785A1, published Dec. 13, 2018, entitled “Self-regulating aav vectors for safe expression of mecp2 in rett syndrome”; US20210180085A1, published Jun. 17, 2021, entitled “Transgene cassettes designed to express a human mecp2 gene”; WO2023010135A1, published Feb. 2, 2023, entitled “Compositions and methods for modulating expression of methyl-cpg binding protein 2 (mecp2)”; U.S. Ser. No. 11/680,275B2, published Jun. 20, 2023, entitled “Self-regulating AAV vectors for safe expression of MeCP2 in rett syndrome”; WO2023049477A2, published Mar. 30, 2023, entitled “Compositions for editing mecp2 transcripts and methods thereof”; the entire contents of each of which are herein incorporated by reference.

Certain gene therapies provided in this section may be useful in treating Rett syndrome by modulating the activity of genes and gene products other than MECP2 genes/gene products, such as other genes/gene products associated with Rett syndrome.

### Linkers

Complexes described herein generally comprise a linker that covalently links any one of the anti-TfR1 antibodies described herein to a molecular payload. A linker comprises at least one covalent bond. In some embodiments, a linker may be a single bond, e.g., a disulfide bond or disulfide bridge, that covalently links an anti-TfR1 antibody to a molecular payload. However, in some embodiments, a linker may covalently link any one of the anti-TfR1 antibodies described herein to a molecular payload through multiple covalent bonds. In some embodiments, a linker may be a cleavable linker. However, in some embodiments, a linker may be a non-cleavable linker. A linker is typically stable in vitro and in vivo, and may be stable in certain cellular environments. Additionally, typically a linker does not negatively impact the functional properties of either the anti-TfR1 antibody or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493.; Jain, N. et al. “Current ADC Linker Chemistry” Pharm Res. 2015, 32:11, 3526-3540.; McCombs, J. R. and Owen, S. C. “Antibody Drug Conjugates: Design and Selection of Linker, Payload and Conjugation Chemistry” AAPS J. 2015, 17:2, 339-351.).

A linker typically will contain two different reactive species that allow for attachment to both the anti-TfR1 antibody and a molecular payload. In some embodiments, the two different reactive species may be a nucleophile and/or an electrophile. In some embodiments, a linker contains two different electrophiles or nucleophiles that are specific for two different nucleophiles or electrophiles. In some embodiments, a linker is covalently linked to an anti-TfR1 antibody via conjugation to a lysine residue or a cysteine residue of the anti-TfR1 antibody. In some embodiments, a linker is covalently linked to a cysteine residue of an anti-TfR1 antibody via a maleimide-containing linker, wherein optionally the maleimide-containing linker comprises a maleimidocaproyl or maleimidomethyl cyclohexane-1-carboxylate group. In some embodiments, a linker is covalently linked to a cysteine residue of an anti-TfR1 antibody or thiol functionalized molecular payload via a 3-arylpropionitrile functional group. In some embodiments, a linker is covalently linked to a lysine residue of an anti-TfR1 antibody. In some embodiments, a linker is covalently linked to an anti-TfR1 antibody and/or (e.g., and) a molecular payload, independently, via an amide bond, a carbamate bond, a hydrazide, a triazole, a thioether, and/or a disulfide bond.

The linker structures described herein may contain asymmetric or chiral centers, and, therefore, exist in different stereoisomeric forms. The present description is intended to include all stereoisomeric forms of the linker structures described herein, including enantiomeric forms (which may exist even in the absence of asymmetric carbons), rotameric forms, atropisomers, and diastereomeric forms, as well as mixtures thereof, including racemic mixtures.

i. Cleavable Linkers

A cleavable linker may be a protease-sensitive linker, a pH-sensitive linker, or a glutathione-sensitive linker. These linkers are typically cleavable only intracellularly and are preferably stable in extracellular environments, e.g., extracellular to a CNS cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline sequence. In some embodiments, a protease-sensitive linker can be cleaved by a lysosomal protease, e.g. cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g., a cysteine residue.

In some embodiments, a linker comprises a valine-citrulline sequence (e.g., as described in U.S. Pat. No. 6,214,345, incorporated herein by reference). In some embodiments, before conjugation, a linker comprises a structure of:

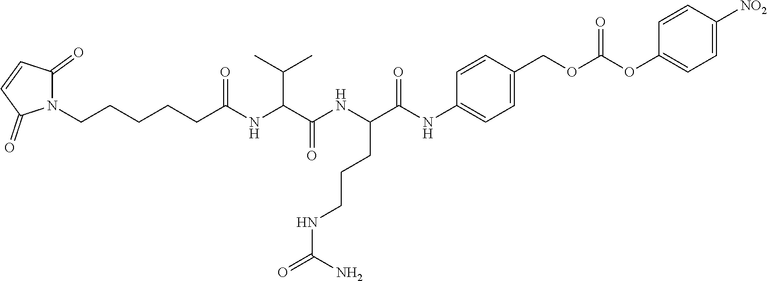 [SMILES: CC(C)C(NC(=O)CCCCCN1C(=O)C=CC1=O)C(=O)NC(CCCNC(N)=O)C(=O)Nc1ccc(COC(=O)Oc2ccc([N+](=O)[O-])cc2)cc1]

In some embodiments, after conjugation, a linker comprises a structure of:

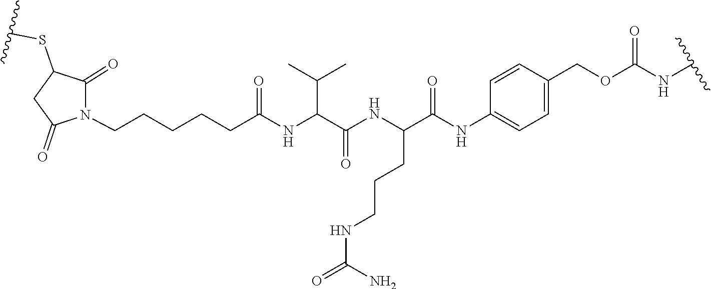 [SMILES: *NC(=O)OCc1ccc(NC(=O)C(CCCNC(N)=O)NC(=O)C(NC(=O)CCCCCN2C(=O)CC(S*)C2=O)C(C)C)cc1]

In some embodiments, before conjugation, a linker comprises a structure of:

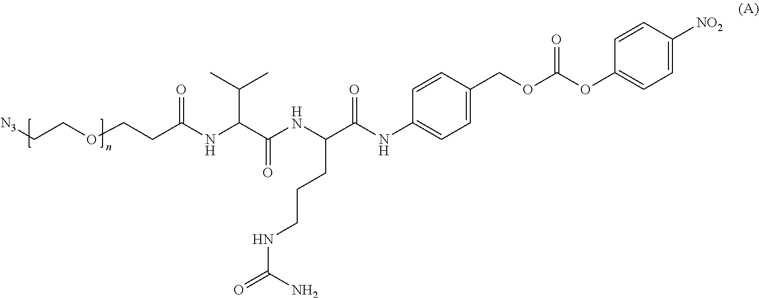

wherein n is any number from 0-10. In some embodiments, n is 3.

In some embodiments, a linker comprises a structure of:

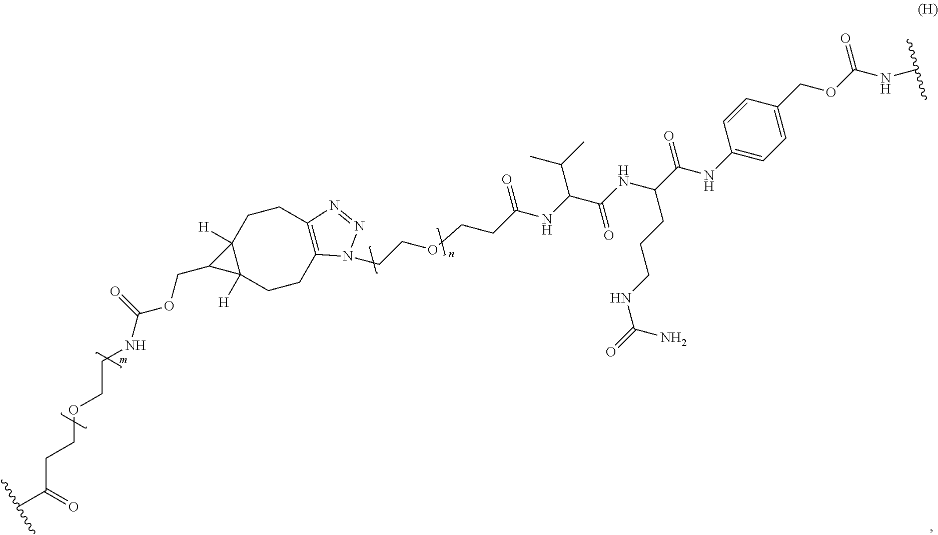

wherein n is any number from 0-10, wherein m is any number from 0-10. In some embodiments, n is 3 and/or (e.g., and) m is 4.

In some embodiments, a linker comprises a structure of:

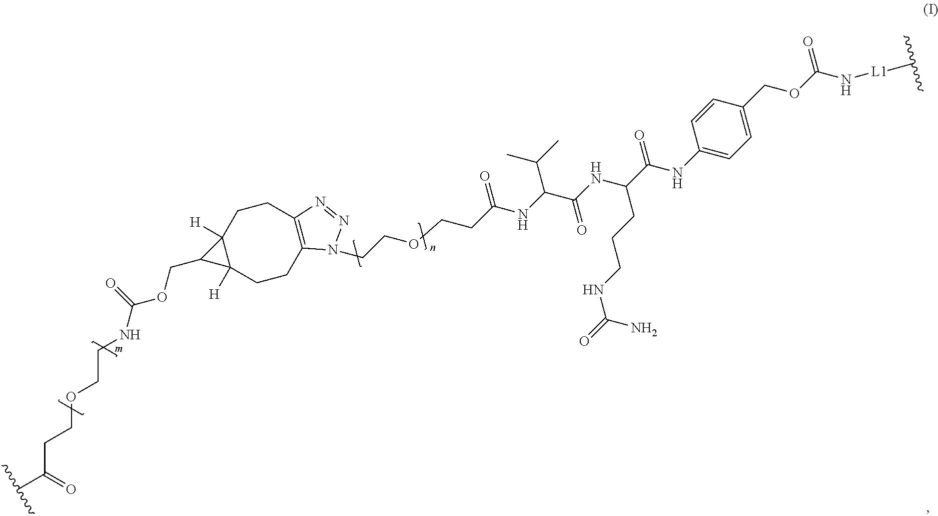

wherein n is any number from 0-10, wherein m is any number from 0-10. In some embodiments, n is 3 and/or (e.g., and) m is 4.

In some embodiments, L1 is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, S(O)₂NR^{A}—, —NR^{A}S(O)₂—, or a combination thereof, wherein each R^{A} is independently hydrogen or substituted or unsubstituted alkyl. In some embodiments, L1 is

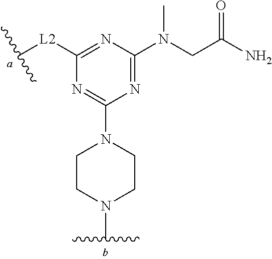

wherein L2 is

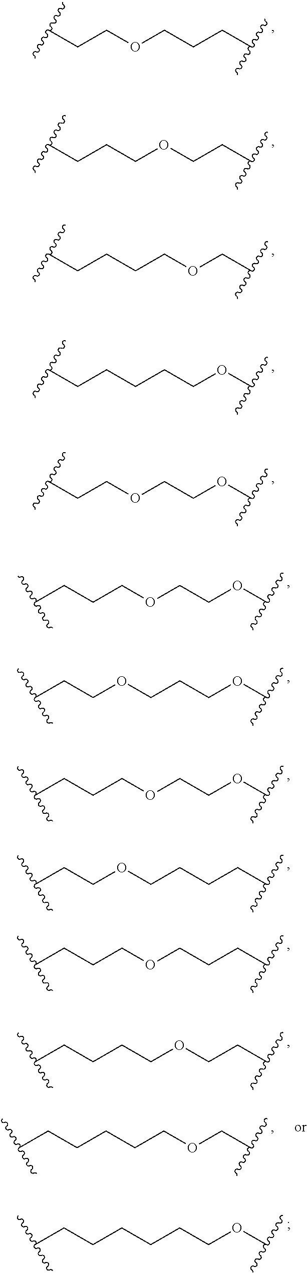

wherein a labels the site directly linked to the carbamate moiety of formulae (B), (D), (E), and (I); and b labels the site covalently linked (directly or via additional chemical moieties) to the molecular payload.

In some embodiments, L1 is:

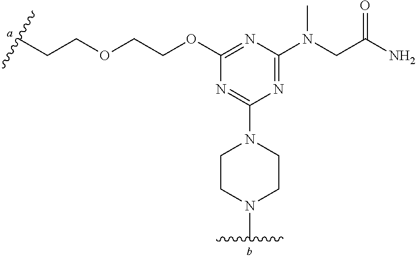 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the molecular payload.

In some embodiments, L1 is

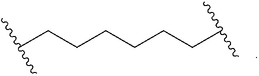 [SMILES: *CCCCCC*]

In some embodiments, L1 is linked to a 5′ phosphate of the molecular payload (e.g., oligonucleotide). In some embodiments, the phosphate is a phosphodiester. In some embodiments, L1 is linked to a 5′ phosphorothioate of the oligonucleotide. In some embodiments, L1 is linked to a 5′ phosphonoamidate of the oligonucleotide. In some embodiments, L1 is linked via a phosphorodiamidate linkage to the 5′ end of the oligonucleotide.

In some embodiments, L1 is optional (e.g., need not be present).

ii. Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation can be utilized to covalently link an anti-TfR1 antibody comprising a LPXT sequence to a molecular payload comprising a (G)n sequence (see, e.g. Proft T. Sortase-mediated protein ligation: an emerging biotechnology tool for protein modification and immobilization. Biotechnol Lett. 2010, 32(1):1-10.).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S, an imino, an optionally substituted nitrogen species, an optionally substituted oxygen species 0, an optionally substituted sulfur species, or a poly(alkylene oxide), e.g. polyethylene oxide or polypropylene oxide. In some embodiments, a linker may be a non-cleavable N-gamma-maleimidobutyryl-oxysuccinimide ester (GMBS) linker.

iii. Linker Conjugation

In some embodiments, a linker is covalently linked to an anti-TfR1 antibody and/or (e.g., and) molecular payload via a phosphate, thioether, ether, carbon-carbon, carbamate, or amide bond. In some embodiments, a linker is covalently linked to a molecular payload (e.g., an oligonucleotide) through a phosphate or phosphorothioate group, e.g. a terminal phosphate of an oligonucleotide backbone. In some embodiments, a linker is covalently linked to an anti-TfR1 antibody, through a lysine or cysteine residue present on the anti-TfR1 antibody.

In some embodiments, a linker, or a portion thereof is covalently linked to an anti-TfR1 antibody and/or (e.g., and) molecular payload by a cycloaddition reaction between an azide and an alkyne to form a triazole, wherein the azide or the alkyne may be located on the anti-TfR1 antibody, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctyne is as described in International Patent Application Publication WO2011136645, published on Nov. 3, 2011, entitled, “Fused Cyclooctyne Compounds And Their Use In Metal-free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some embodiments, a cycloaddition reaction between an azide and an alkyne to form a triazole, wherein the azide or the alkyne may be located on the anti-TfR1 antibody, molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”.

In some embodiments, a linker comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is covalently linked to an anti-TfR1 antibody and/or (e.g., and) molecular payload by the Diels-Alder reaction between a dienophile and a diene/hetero-diene, wherein the dienophile or the diene/hetero-diene may be located on the anti-TfR1 antibody, molecular payload, or the linker. In some embodiments a linker is covalently linked to an anti-TfR1 antibody and/or (e.g., and) molecular payload by other pericyclic reactions such as an ene reaction. In some embodiments, a linker is covalently linked to an anti-TfR1 antibody and/or (e.g., and) molecular payload by an amide, thioamide, or sulfonamide bond reaction. In some embodiments, a linker is covalently linked to an anti-TfR1 antibody and/or (e.g., and) molecular payload by a condensation reaction to form an oxime, hydrazone, or semicarbazide group existing between the linker and the anti-TfR1 antibody and/or (e.g., and) molecular payload.

In some embodiments, a linker is covalently linked to an anti-TfR1 antibody and/or (e.g., and) molecular payload by a conjugate addition reaction between a nucleophile, e.g. an amine or a hydroxyl group, and an electrophile, e.g. a carboxylic acid, carbonate, or an aldehyde. In some embodiments, a nucleophile may exist on a linker and an electrophile may exist on an anti-TfR1 antibody or molecular payload prior to a reaction between a linker and an anti-TfR1 antibody or molecular payload. In some embodiments, an electrophile may exist on a linker and a nucleophile may exist on an anti-TfR1 antibody or molecular payload prior to a reaction between a linker and an anti-TfR1 antibody or molecular payload. In some embodiments, an electrophile may be an azide, pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, and/or a thiol group.

In some embodiments, a linker comprises a valine-citrulline sequence covalently linked to a reactive chemical moiety (e.g., an azide moiety or a BCN moiety for click chemistry). In some embodiments, a linker comprising a valine-citrulline sequence covalently linked to a reactive chemical moiety (e.g., an azide moiety for click chemistry) comprises a structure of:

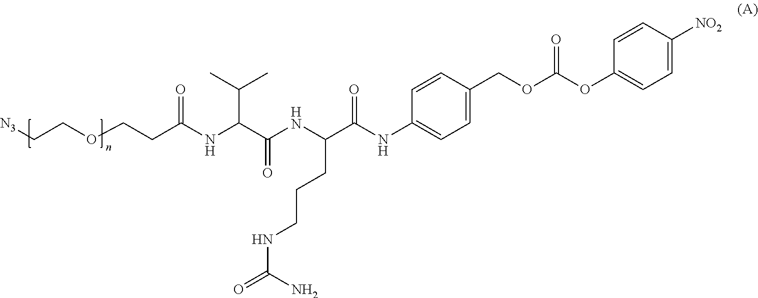

wherein n is any number from 0-10. In some embodiments, n is 3.

In some embodiments, a linker comprising the structure of Formula (A) is covalently linked (e.g., optionally via additional chemical moieties) to a molecular payload (e.g., an oligonucleotide, polypeptide, small molecule, or gene therapy payload). In some embodiments, a linker comprising the structure of Formula (A) is covalently linked to a molecular payload, e.g., through a nucleophilic substitution with amine-L1-molecular payloads forming a carbamate bond, yielding a compound comprising a structure of:

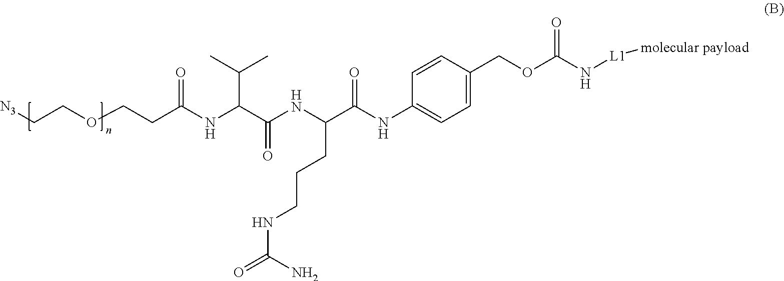

wherein n is any number from 0-10. In some embodiments, n is 3.

In some embodiments, the compound of Formula (B) is further covalently linked via a triazole to additional moieties, wherein the triazole is formed by a click reaction between the azide of Formula (A) or Formula (B) and an alkyne provided on a bicyclononyne. In some embodiments, a compound comprising a bicyclononyne comprises a structure of:

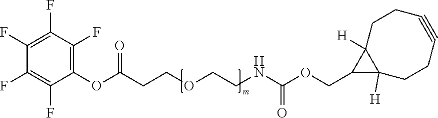

wherein m is any number from 0-10. In some embodiments, m is 4.

In some embodiments, the azide of the compound of structure (B) forms a triazole via a click reaction with the alkyne of the compound of structure (C), forming a compound comprising a structure of:

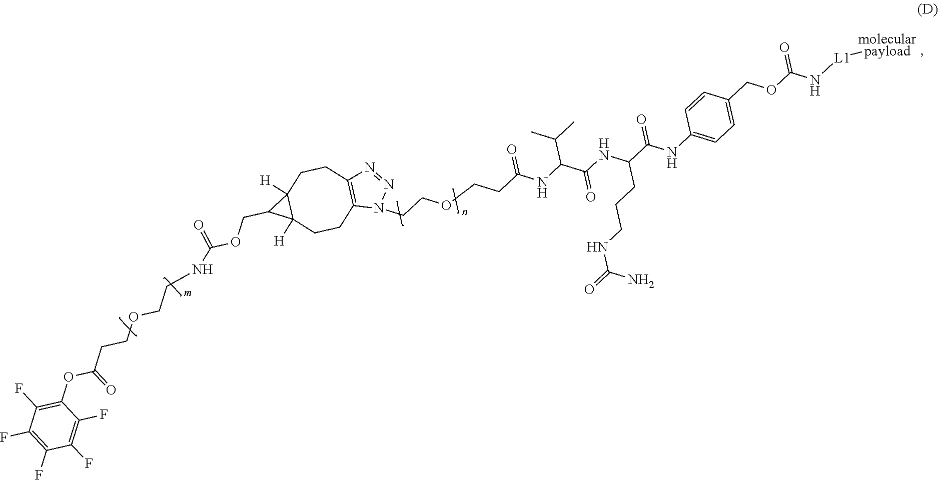

wherein n is any number from 0-10, and wherein m is any number from 0-10. In some embodiments, n is 3 and m is 4.

In some embodiments, the compound of structure (D) is further covalently linked to a lysine of the anti-TfR1 antibody, forming a complex comprising a structure of:

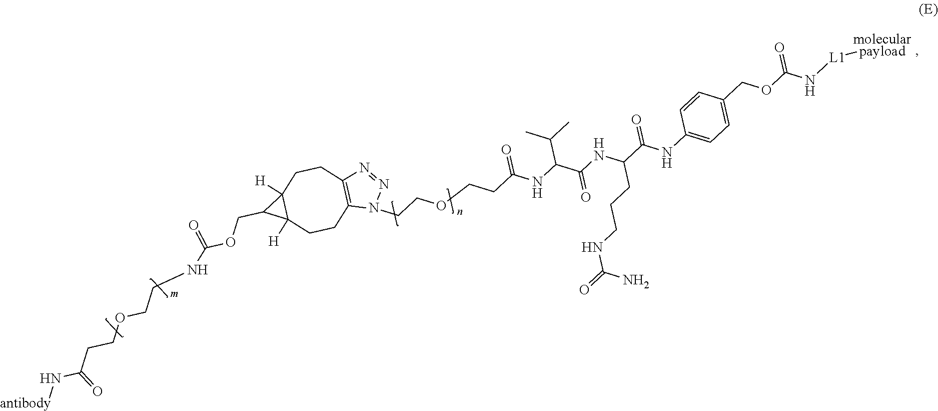

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the compound of Formula (C) is further covalently linked to a lysine of the anti-TfR1 antibody, forming a compound comprising a structure of:

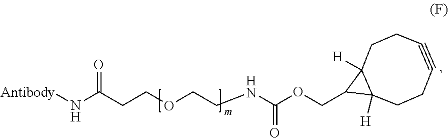

wherein m is 0-15 (e.g., 4). It should be understood that the amide shown adjacent the anti-TfR1 antibody in Formula (F) results from a reaction with an amine of the anti-TfR1 antibody, such as a lysine epsilon amine.

In some embodiments, the azide of the compound of structure (B) forms a triazole via a click reaction with the alkyne of the compound of structure (F), forming a complex comprising a structure of:

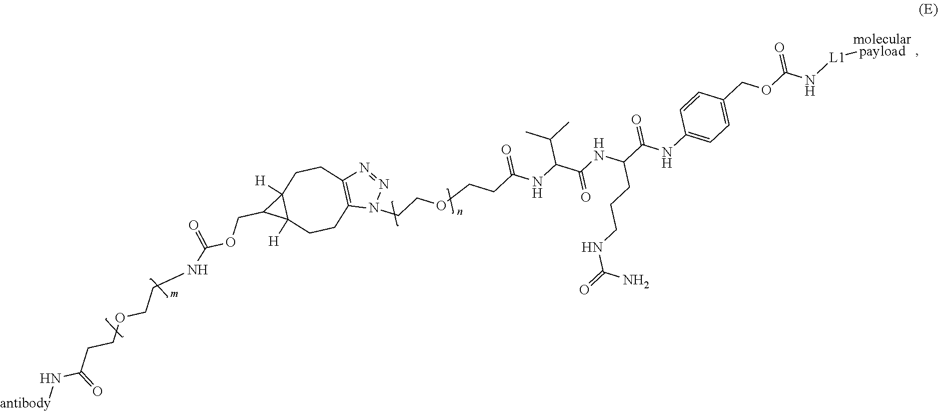

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the azide of the compound of structure (A) forms a triazole via a click reaction with the alkyne of the compound of structure (F), forming a compound comprising a structure of:

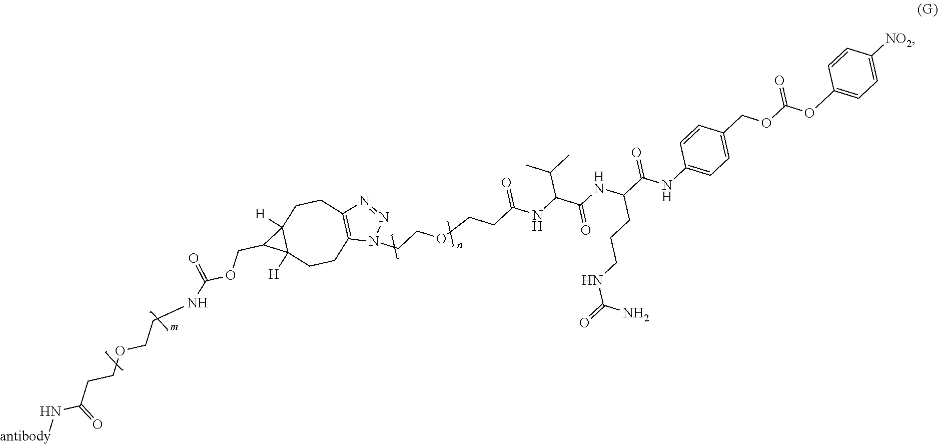

wherein n is any number from 0-10, wherein m is any number from 0-10. In some embodiments, n is 3 and/or (e.g., and) m is 4. In some embodiments, an oligonucleotide is covalently linked to a compound comprising a structure of formula (G), thereby forming a complex comprising a structure of formula (E). It should be understood that the amide shown adjacent the anti-TfR1 antibody in Formula (G) results from a reaction with an amine of the anti-TfR1 antibody, such as a lysine epsilon amine.

In some embodiments, in any one of the complexes described herein, the anti-TfR1 antibody is covalently linked via a lysine of the anti-TfR1 antibody to a molecular payload (e.g., an oligonucleotide, polypeptide, small molecule, or gene therapy payload) via a linker comprising a structure of:

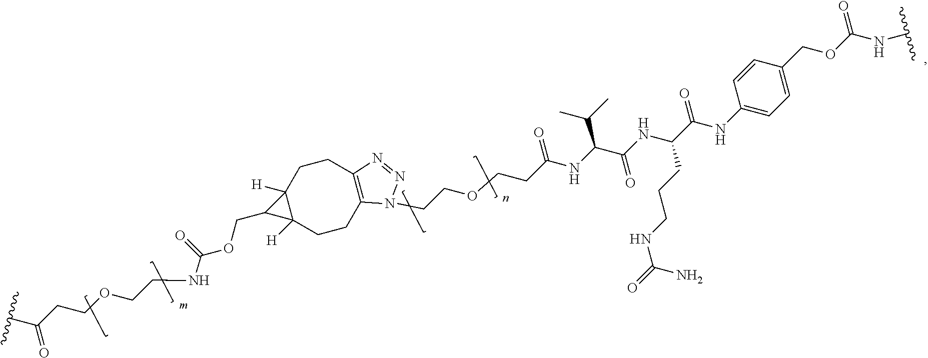

wherein n is any number from 0-10, wherein m is any number from 0-10. In some embodiments, n is 3 and/or (e.g., and) m is 4.

In some embodiments, in any one of the complexes described herein, the anti-TfR1 antibody is covalently linked via a lysine of the anti-TfR1 antibody to a molecular payload (e.g., an oligonucleotide, polypeptide, small molecule, or gene therapy payload) via a linker comprising a structure of:

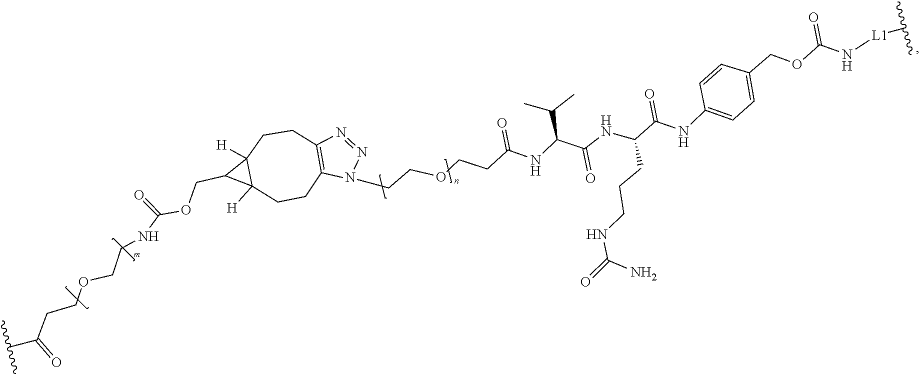

wherein n is any number from 0-10, wherein m is any number from 0-10. In some embodiments, n is 3 and/or (e.g., and) m is 4.

In some embodiments, in formulae (B), (D), (E), and (I), L1 is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, S(O)₂NR^{A}—, —NR^{A}S(O)₂—, or a combination thereof, wherein each R^{A} is independently hydrogen or substituted or unsubstituted alkyl. In some embodiments, L1 is

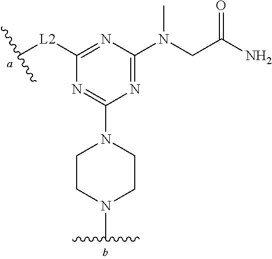

wherein L2 is

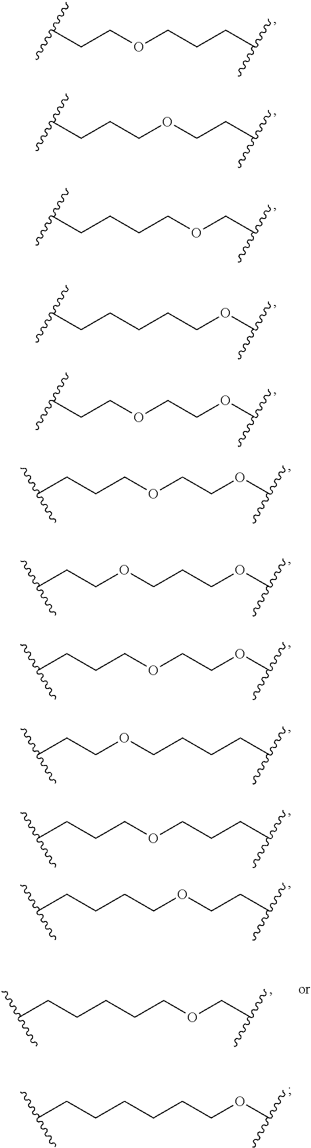

wherein a labels the site directly linked to the carbamate moiety of formulae (B), (D), (E), and (I); and b labels the site covalently linked (directly or via additional chemical moieties) to the molecular payload.

In some embodiments, L1 is:

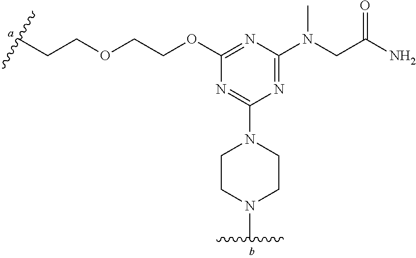 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the molecular payload.

In some embodiments, L1 is

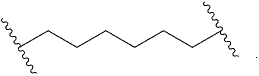 [SMILES: *CCCCCC*]

In some embodiments, L1 is linked to a 5′ phosphate of the molecular payload (e.g., oligonucleotide). In some embodiments, the phosphate is a phosphodiester. In some embodiments, L1 is linked to a 5′ phosphorothioate of the oligonucleotide. In some embodiments, L1 is linked to a 5′ phosphonoamidate of the oligonucleotide. In some embodiments, L1 is linked via a phosphorodiamidate linkage to the 5′ end of the oligonucleotide.

In some embodiments, L1 is optional (e.g., need not be present).

In some embodiments, any one of the complexes described herein has a structure of:

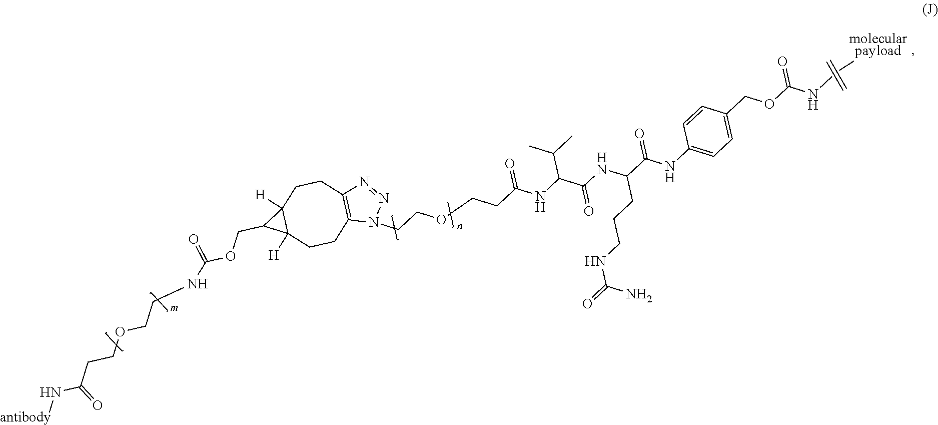

wherein n is 0-15 (e.g., 3) and m is 0-15 (e.g., 4). It should be understood that the amide shown adjacent the anti-TfR1 antibody in Formula (J) results from a reaction with an amine of the anti-TfR1 antibody, such as a lysine epsilon amine.

In some embodiments, any one of the complexes described herein has a structure of:

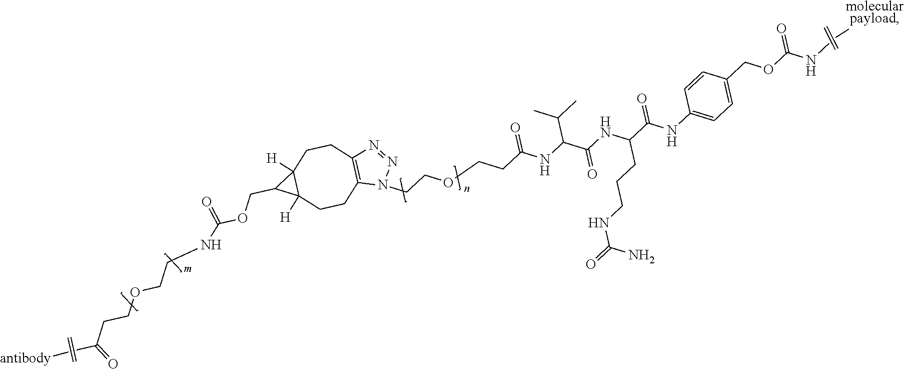

wherein n is 0-15 (e.g., 3) and m is 0-15 (e.g., 4).

In some embodiments, the molecular payload is modified to comprise an amine group, e.g., at the 5′ end, the 3′ end, or internally (e.g., as an amine functionalized nucleobase) in an oligonucleotide payload, prior to linking to a compound, e.g., a compound of formula (A) or formula (G).

Although linker conjugation is described in the context of anti-TfR1 antibodies and molecular payloads (e.g., oligonucleotides), it should be understood that use of such linker conjugation on other CNS-targeting agents, such as other CNS-targeting antibodies, and/or on other molecular payloads is contemplated.

### Examples of Antibody-Molecular Payload Complexes

Further provided herein are non-limiting examples of complexes comprising any one the anti-TfR1 antibodies described herein covalently linked to any of the molecular payloads (e.g., an oligonucleotide, polypeptide, small molecule, or gene therapy payload) described herein. In some embodiments, the anti-TfR1 antibody (e.g., an anti-TfR1 antibody provided in Table 2) is covalently linked to a molecular payload (e.g., an oligonucleotide, polypeptide, small molecule, or gene therapy payload disclosed herein) via a linker. Any of the linkers described herein may be used. In some embodiments, if the molecular payload comprises an oligonucleotide, the linker is linked to the 5′ end of the oligonucleotide, the 3′ end of the oligonucleotide, or to an internal site of the oligonucleotide. In some embodiments, if the molecular payload comprises a double-stranded oligonucleotide (e.g., a siRNA) comprising a sense strand and an antisense strand, the linker is linked to the 5′ end of the sense strand or the antisense strand, the 3′ end of the sense strand or the antisense strand, or to an internal site of the sense strand or the antisense strand. In some embodiments, the linker is linked to the anti-TfR1 antibody via a thiol-reactive linkage (e.g., via a cysteine in the anti-TfR1 antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a molecular payload disclosed herein (e.g., an oligonucleotide, polypeptide, small molecule, or gene therapy disclosed herein). In some embodiments, the molecular payload comprises an oligonucleotide. In some embodiments, the molecular payload comprises an oligonucleotide comprising a region of complementarity to any one of the target genes described herein (e.g., DMPK, DMD, SMN, FXN, SOD1, C9orf72, ATXN2, FUS, LRRK2, SCNA, HTT, MSH3, TREM2, APOE, MAPT, APP, GYS1, PrP, VLA-4, UBE3A, GFAP, LSD, SCN9A, SCN1A, SCN2A, SCN8A, CLN3, GRIA1, and PCDH19). In some embodiments, the molecular payload comprises an oligonucleotide comprising a region of complementarity to any one of the target genes described herein (e.g., TOR1A, THAP1, ANO3, GNAL, KMT2B, GCH1, TH, SPR, TAF1, PRKRA, ATP1A3, SGCE, PNKD, PRRT2, SLC2A1, and ECHS1). In some embodiments, the molecular payload comprises an oligonucleotide comprising a region of complementarity to any one of the target genes described herein (e.g., PIKFYVE, SYF2, UNC13A, ATXN1, ATXN3, GRN, GRIN2A, TPP1, GLB1, ASM, ARSA, GALC, HEXA, HEXB, GBA, and MECP2). In some embodiments, the molecular payload comprises an oligonucleotide comprising a region of complementarity to a target sequence as set forth in any one of SEQ ID NOs: 392-702. In some embodiments, the molecular payload comprises an oligonucleotide comprising a region of complementarity to a target sequence as set forth in any one of SEQ ID NOs: 705-803. In some embodiments, the molecular payload comprises an oligonucleotide comprising a region of complementarity to a target sequence as set forth in any one of SEQ ID NOs: 143-148, 167-169, 810-875, and 1059-1068. In some embodiments, the molecular payload comprises an oligonucleotide comprising a nucleobase sequence of any one of the oligonucleotide molecular payloads provided in Tables 5-19 and optionally comprising any suitable modifications (e.g., modified nucleosides and/or internucleoside linkages). In some embodiments, the molecular payload comprises an oligonucleotide disclosed in any one of Tables 5-19.

An example of a structure of a complex comprising an anti-TfR1 antibody covalently linked to a molecular payload via a linker is provided below:

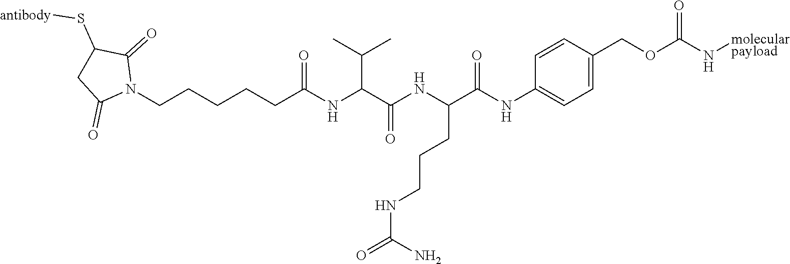

wherein the linker is linked to the antibody via a thiol-reactive linkage (e.g., via a cysteine in the antibody). In some embodiments, the molecular payload is a molecular payload disclosed herein (e.g., an oligonucleotide, polypeptide, small molecule, or gene therapy disclosed herein). In some embodiments, the molecular payload comprises an oligonucleotide. In some embodiments, the molecular payload comprises an oligonucleotide comprising a region of complementarity to any one of the target genes described herein (e.g., DMPK, DMD, SMN, FXN, SOD1, C9orf72, ATXN2, FUS, LRRK2, SCNA, HTT, MSH3, TREM2, APOE, MAPT, APP, GYS1, PrP, VLA-4, UBE3A, GFAP, LSD, SCN9A, SCN1A, SCN2A, SCN8A, CLN3, GRIA1, and PCDH19). In some embodiments, the molecular payload comprises an oligonucleotide comprising a region of complementarity to any one of the target genes described herein (e.g., TOR1A, THAP1, ANO3, GNAL, KMT2B, GCH1, TH, SPR, TAF1, PRKRA, ATP1A3, SGCE, PNKD, PRRT2, SLC2A1, and ECHS1). In some embodiments, the molecular payload comprises an oligonucleotide comprising a region of complementarity to any one of the target genes described herein (e.g., PIKFYVE, SYF2, UNC13A, ATXN1, ATXN3, GRN, GRIN2A, TPP1, GLB1, ASM, ARSA, GALC, HEXA, HEXB, GBA, and MECP2). In some embodiments, the molecular payload comprises an oligonucleotide comprising a region of complementarity to a target sequence as set forth in any one of SEQ ID NOs: 392-702. In some embodiments, the molecular payload comprises an oligonucleotide comprising a region of complementarity to a target sequence as set forth in any one of SEQ ID NOs: 705-803. In some embodiments, the molecular payload comprises an oligonucleotide comprising a region of complementarity to a target sequence as set forth in any one of SEQ ID NOs: 143-148, 167-169, 810-875, and 1059-1068. In some embodiments, the molecular payload comprises an oligonucleotide comprising a nucleobase sequence of any one of the oligonucleotide molecular payloads provided in Tables 5-19 and optionally comprising any suitable modifications (e.g., modified nucleosides and/or internucleoside linkages). In some embodiments, the molecular payload comprises an oligonucleotide disclosed in any one of Tables 5-19.

Another example of a structure of a complex comprising an anti-TfR1 antibody covalently linked to an oligonucleotide via a linker is provided below:

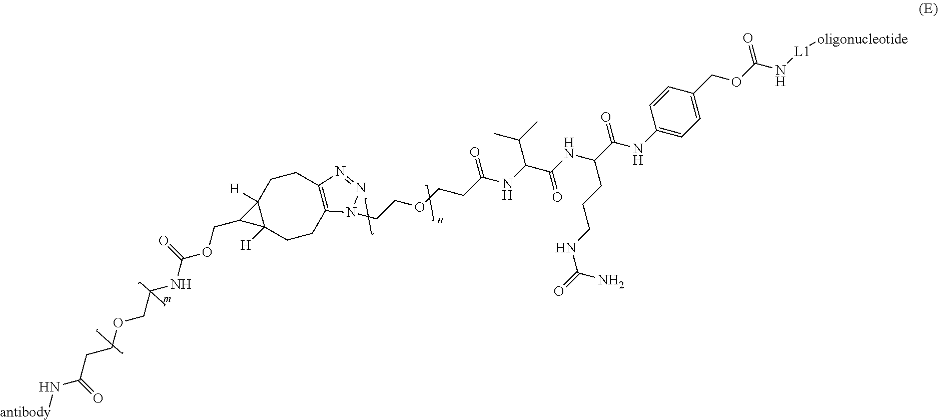

wherein n is a number between 0-10, wherein m is a number between 0-10, wherein the linker is linked to the antibody via an amine group (e.g., on a lysine residue), and/or (e.g., and) wherein the linker is linked to the oligonucleotide (e.g., at the 5′ end, 3′ end, or internally). In some embodiments, L1 is as defined above. In some embodiments, the linker is linked to the antibody via a lysine, the linker is linked to the oligonucleotide at the 5′ end, n is 3, and m is 4. In some embodiments, the molecular payload is a molecular payload disclosed herein (e.g., an oligonucleotide, polypeptide, small molecule, or gene therapy disclosed herein). In some embodiments, the molecular payload comprises an oligonucleotide. In some embodiments, the molecular payload comprises an oligonucleotide comprising a region of complementarity to any one of the target genes described herein (e.g., DMPK, DMD, SMN, FXN, SOD1, C9orf72, ATXN2, FUS, LRRK2, SCNA, HTT, MSH3, TREM2, APOE, MAPT, APP, GYS1, PrP, VLA-4, UBE3A, GFAP, LSD, SCN9A, SCN1A, SCN2A, SCN8A, CLN3, GRIA1, and PCDH19). In some embodiments, the molecular payload comprises an oligonucleotide comprising a region of complementarity to any one of the target genes described herein (e.g., TOR1A, THAP1, ANO3, GNAL, KMT2B, GCH1, TH, SPR, TAF1, PRKRA, ATP1A3, SGCE, PNKD, PRRT2, SLC2A1, and ECHS1). In some embodiments, the molecular payload comprises an oligonucleotide comprising a region of complementarity to any one of the target genes described herein (e.g., PIKFYVE, SYF2, UNC13A, ATXN1, ATXN3, GRN, GRIN2A, TPP1, GLB1, ASM, ARSA, GALC, HEXA, HEXB, GBA, and MECP2). In some embodiments, the molecular payload comprises an oligonucleotide comprising a region of complementarity to a target sequence as set forth in any one of SEQ ID NOs: 392-702. In some embodiments, the molecular payload comprises an oligonucleotide comprising a region of complementarity to a target sequence as set forth in any one of SEQ ID NOs: 705-803. In some embodiments, the molecular payload comprises an oligonucleotide comprising a region of complementarity to a target sequence as set forth in any one of SEQ ID NOs: 143-148, 167-169, 810-875, and 1059-1068. In some embodiments, the molecular payload comprises an oligonucleotide comprising a nucleobase sequence of any one of the oligonucleotide molecular payloads provided in Tables 5-19 and optionally comprising any suitable modifications (e.g., modified nucleosides and/or internucleoside linkages). In some embodiments, the molecular payload comprises an oligonucleotide disclosed in any one of Tables 5-19. It should be understood that the amide shown adjacent the anti-TfR1 antibody in Formula (E) results from a reaction with an amine of the anti-TfR1 antibody, such as a lysine epsilon amine.

It should be appreciated that antibodies can be linked to molecular payloads with different stoichiometries, a property that may be referred to as a drug to antibody ratios (DAR) with the “drug” being the molecular payload. In some embodiments, one molecular payload is linked to an antibody (DAR=1). In some embodiments, two molecular payloads are linked to an antibody (DAR=2). In some embodiments, three molecular payloads are linked to an antibody (DAR=3). In some embodiments, four molecular payloads are linked to an antibody (DAR=4). In some embodiments, a mixture of different complexes, each having a different DAR, is provided. In some embodiments, an average DAR of complexes in such a mixture may be in a range of 1-10 (e.g., 1-10, 1-8, 1-6, 1-4, 1-2, 2-10, 2-8, 2-6, 2-4, 4-10, 4-8, 4-6, 6-10, 6-8, or 8-10). In some embodiments, an average DAR of complexes in such a mixture may be in a range of 1 to 3, 1 to 4, 1 to 5 or more. DAR may be increased by conjugating molecular payloads to different sites on an antibody and/or (e.g., and) by conjugating multimers to one or more sites on antibody. For example, a DAR of 2 may be achieved by conjugating a single molecular payload to two different sites on an antibody or by conjugating a dimer molecular payload to a single site of an antibody.

In some embodiments, the complex described herein comprises an anti-TfR1 antibody described herein (e.g., an antibody provided in Table 2) covalently linked to a molecular payload. In some embodiments, the complex described herein comprises an anti-TfR1 antibody described herein (e.g., an antibody provided in Table 2) covalently linked to molecular payload via a linker (e.g., a linker comprising a valine-citrulline sequence). In some embodiments, the linker (e.g., a linker comprising a valine-citrulline sequence) is linked to the antibody (e.g., an anti-TfR1 antibody described herein) via a thiol-reactive linkage (e.g., via a cysteine in the antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a molecular payload disclosed herein (e.g., an oligonucleotide, polypeptide, small molecule, or gene therapy disclosed herein). In some embodiments, the molecular payload comprises an oligonucleotide. In some embodiments, the molecular payload comprises an oligonucleotide comprising a region of complementarity to any one of the target gene described herein (e.g., DMPK, DMD, SMN, FXN, SOD1, C9orf72, ATXN2, FUS, LRRK2, SCNA, HTT, MSH3, TREM2, APOE, MAPT, APP, GYS1, PrP, VLA-4, UBE3A, GFAP, LSD, SCN9A, SCN1A, SCN2A, SCN8A, CLN3, GRIA1, and PCDH19). In some embodiments, the molecular payload comprises an oligonucleotide comprising a region of complementarity to any one of the target gene described herein (e.g., TOR1A, THAP1, ANO3, GNAL, KMT2B, GCH1, TH, SPR, TAF1, PRKRA, ATP1A3, SGCE, PNKD, PRRT2, SLC2A1, and ECHS1). In some embodiments, the molecular payload comprises an oligonucleotide comprising a region of complementarity to any one of the target genes described herein (e.g., PIKFYVE, SYF2, UNC13A, ATXN1, ATXN3, GRN, GRIN2A, TPP1, GLB1, ASM, ARSA, GALC, HEXA, HEXB, GBA, and MECP2). In some embodiments, the molecular payload comprises an oligonucleotide comprising a region of complementarity to a target sequence as set forth in any one of SEQ ID NOs: 392-702. In some embodiments, the molecular payload comprises an oligonucleotide comprising a region of complementarity to a target sequence as set forth in any one of SEQ ID NOs: 705-803. In some embodiments, the molecular payload comprises an oligonucleotide comprising a region of complementarity to a target sequence as set forth in any one of SEQ ID NOs: 143-148, 167-169, 810-875, and 1059-1068. In some embodiments, the molecular payload comprises an oligonucleotide comprising a nucleobase sequence of any one of the oligonucleotide molecular payloads provided in Tables 5-19 and optionally comprising any suitable modifications (e.g., modified nucleosides and/or internucleoside linkages). In some embodiments, the molecular payload comprises an oligonucleotide disclosed in any one of Tables 5-19.

In some embodiments, the complex described herein comprises an anti-TfR1 antibody covalently linked to a molecular payload, wherein the anti-TfR1 antibody comprises a CDR-H1, a CDR-H2, a CDR-H3, a CDR-L1, a CDR-L2, and a CDR-L3 listed in Table 2. In some embodiments, the molecular payload is a molecular payload disclosed herein (e.g., an oligonucleotide, polypeptide, small molecule, or gene therapy disclosed herein). In some embodiments, the molecular payload comprises an oligonucleotide. In some embodiments, the molecular payload comprises an oligonucleotide comprising a region of complementarity to any one of the target gene described herein (e.g., DMPK, DMD, SMN, FXN, SOD1, C9orf72, ATXN2, FUS, LRRK2, SCNA, HTT, MSH3, TREM2, APOE, MAPT, APP, GYS1, PrP, VLA-4, UBE3A, GFAP, LSD, SCN9A, SCN1A, SCN2A, SCN8A, CLN3, GRIA1, and PCDH19). In some embodiments, the molecular payload comprises an oligonucleotide comprising a region of complementarity to any one of the target gene described herein (e.g., TOR1A, THAP1, ANO3, GNAL, KMT2B, GCH1, TH, SPR, TAF1, PRKRA, ATP1A3, SGCE, PNKD, PRRT2, SLC2A1, and ECHS1). In some embodiments, the molecular payload comprises an oligonucleotide comprising a region of complementarity to any one of the target genes described herein (e.g., PIKFYVE, SYF2, UNC13A, ATXN1, ATXN3, GRN, GRIN2A, TPP1, GLB1, ASM, ARSA, GALC, HEXA, HEXB, GBA, and MECP2). In some embodiments, the molecular payload comprises an oligonucleotide comprising a region of complementarity to a target sequence as set forth in any one of SEQ ID NOs: 392-702. In some embodiments, the molecular payload comprises an oligonucleotide comprising a region of complementarity to a target sequence as set forth in any one of SEQ ID NOs: 705-803. In some embodiments, the molecular payload comprises an oligonucleotide comprising a region of complementarity to a target sequence as set forth in any one of SEQ ID NOs: 143-148, 167-169, 810-875, and 1059-1068. In some embodiments, the molecular payload comprises an oligonucleotide comprising a nucleobase sequence of any one of the oligonucleotide molecular payloads provided in Tables 5-19 and optionally comprising any suitable modifications (e.g., modified nucleosides and/or internucleoside linkages). In some embodiments, the molecular payload comprises an oligonucleotide disclosed in any one of Tables 5-19.

In some embodiments, the complex described herein comprises an anti-TfR1 antibody covalently linked to a molecular payload, wherein the anti-TfR1 antibody comprises a VH comprising the amino acid sequence of SEQ ID NO: 17 and a VL comprising the amino acid sequence of SEQ ID NO: 18. In some embodiments, the molecular payload is a molecular payload disclosed herein (e.g., an oligonucleotide, polypeptide, small molecule, or gene therapy disclosed herein). In some embodiments, the molecular payload comprises an oligonucleotide. In some embodiments, the molecular payload comprises an oligonucleotide comprising a region of complementarity to any one of the target gene described herein (e.g., DMPK, DMD, SMN, FXN, SOD1, C9orf72, ATXN2, FUS, LRRK2, SCNA, HTT, MSH3, TREM2, APOE, MAPT, APP, GYS1, PrP, VLA-4, UBE3A, GFAP, LSD, SCN9A, SCN1A, SCN2A, SCN8A, CLN3, GRIA1, and PCDH19). In some embodiments, the molecular payload comprises an oligonucleotide comprising a region of complementarity to any one of the target gene described herein (e.g., TOR1A, THAP1, ANO3, GNAL, KMT2B, GCH1, TH, SPR, TAF1, PRKRA, ATP1A3, SGCE, PNKD, PRRT2, SLC2A1, and ECHS1). In some embodiments, the molecular payload comprises an oligonucleotide comprising a region of complementarity to any one of the target genes described herein (e.g., PIKFYVE, SYF2, UNC13A, ATXN1, ATXN3, GRN, GRIN2A, TPP1, GLB1, ASM, ARSA, GALC, HEXA, HEXB, GBA, and MECP2). In some embodiments, the molecular payload comprises an oligonucleotide comprising a region of complementarity to a target sequence as set forth in any one of SEQ ID NOs: 392-702. In some embodiments, the molecular payload comprises an oligonucleotide comprising a region of complementarity to a target sequence as set forth in any one of SEQ ID NOs: 705-803. In some embodiments, the molecular payload comprises an oligonucleotide comprising a region of complementarity to a target sequence as set forth in any one of SEQ ID NOs: 143-148, 167-169, 810-875, and 1059-1068. In some embodiments, the molecular payload comprises an oligonucleotide comprising a nucleobase sequence of any one of the oligonucleotide molecular payloads provided in Tables 5-19 and optionally comprising any suitable modifications (e.g., modified nucleosides and/or internucleoside linkages). In some embodiments, the molecular payload comprises an oligonucleotide disclosed in any one of Tables 5-19.

In some embodiments, the complex described herein comprises an anti-TfR1 antibody covalently linked to a molecular payload, wherein the anti-TfR1 antibody comprises a heavy chain comprising the amino acid sequence of SEQ ID NO: 19 and a light chain comprising the amino acid sequence of SEQ ID NO: 20. In some embodiments, the molecular payload is a molecular payload disclosed herein (e.g., an oligonucleotide, polypeptide, small molecule, or gene therapy disclosed herein). In some embodiments, the molecular payload comprises an oligonucleotide. In some embodiments, the molecular payload comprises an oligonucleotide comprising a region of complementarity to any one of the target gene described herein (e.g., DMPK, DMD, SMN, FXN, SOD1, C9orf72, ATXN2, FUS, LRRK2, SCNA, HTT, MSH3, TREM2, APOE, MAPT, APP, GYS1, PrP, VLA-4, UBE3A, GFAP, LSD, SCN9A, SCN1A, SCN2A, SCN8A, CLN3, GRIA1, and PCDH19). In some embodiments, the molecular payload comprises an oligonucleotide comprising a region of complementarity to any one of the target gene described herein (e.g., TOR1A, THAP1, ANO3, GNAL, KMT2B, GCH1, TH, SPR, TAF1, PRKRA, ATP1A3, SGCE, PNKD, PRRT2, SLC2A1, and ECHS1). In some embodiments, the molecular payload comprises an oligonucleotide comprising a region of complementarity to any one of the target genes described herein (e.g., PIKFYVE, SYF2, UNC13A, ATXN1, ATXN3, GRN, GRIN2A, TPP1, GLB1, ASM, ARSA, GALC, HEXA, HEXB, GBA, and MECP2). In some embodiments, the molecular payload comprises an oligonucleotide comprising a region of complementarity to a target sequence as set forth in any one of SEQ ID NOs: 392-702. In some embodiments, the molecular payload comprises an oligonucleotide comprising a region of complementarity to a target sequence as set forth in any one of SEQ ID NOs: 705-803. In some embodiments, the molecular payload comprises an oligonucleotide comprising a region of complementarity to a target sequence as set forth in any one of SEQ ID NOs: 143-148, 167-169, 810-875, and 1059-1068. In some embodiments, the molecular payload comprises an oligonucleotide comprising a nucleobase sequence of any one of the oligonucleotide molecular payloads provided in Tables 5-19 and optionally comprising any suitable modifications (e.g., modified nucleosides and/or internucleoside linkages). In some embodiments, the molecular payload comprises an oligonucleotide disclosed in any one of Tables 5-19.

In any of the example complexes described herein, in some embodiments, the anti-TfR1 antibody is covalently linked to the molecular payload via a linker comprising a structure of:

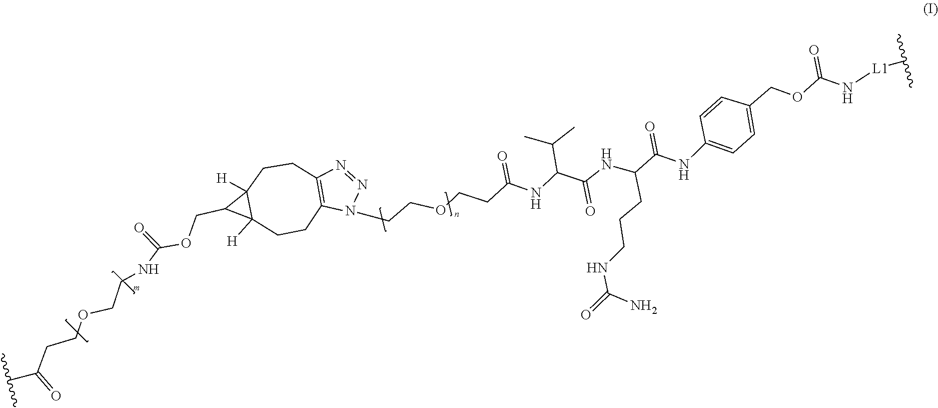

wherein n is 3, m is 4.

In some embodiments, the complex described herein comprises an anti-TfR1 antibody covalently linked to a 5′ end or a 3′ end of an oligonucleotide disclosed herein via a lysine in the anti-TfR1 antibody, wherein the anti-TfR1 antibody comprises a CDR-H1, a CDR-H2, a CDR-H3, a CDR-L1, a CDR-L2, and a CDR-L3 provided in Table 2, wherein the complex has a structure of:

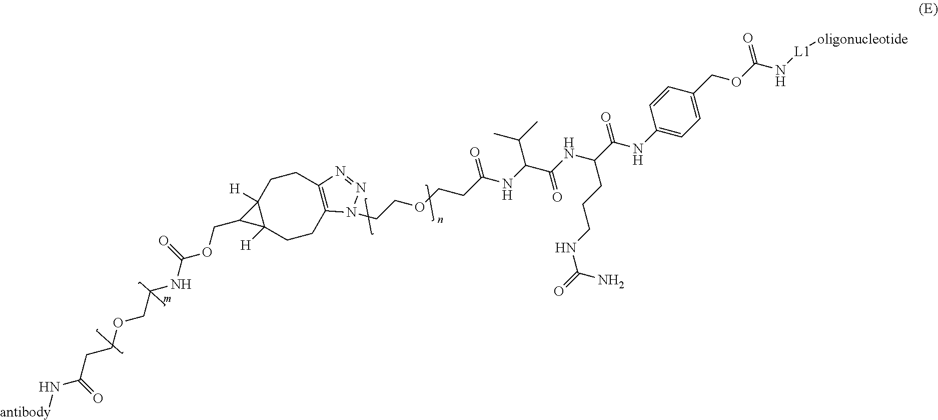

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to a 5′ end or a 3′ end of an oligonucleotide disclosed herein via a lysine in the anti-TfR1 antibody, wherein the anti-TfR1 antibody comprises a VH and VL provided in Table 2, wherein the complex has a structure of:

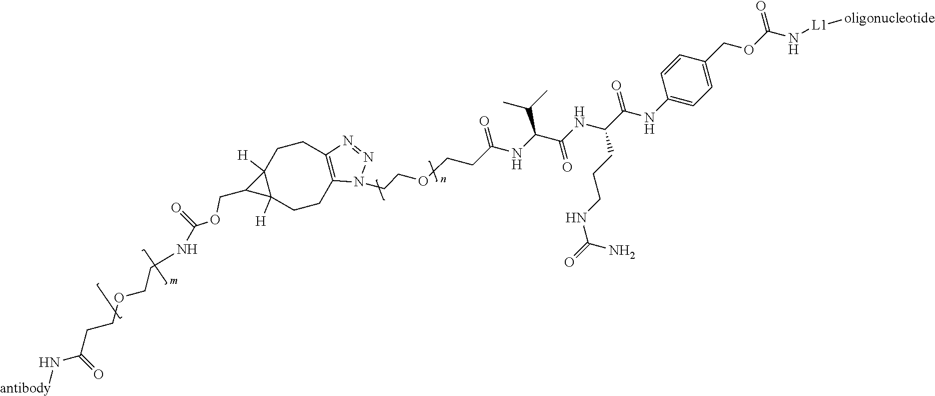

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to a 5′ end or a 3′ end of an oligonucleotide disclosed herein via a lysine in the anti-TfR1 antibody, wherein the anti-TfR1 antibody comprises a heavy chain and light chain of any one of the antibodies listed in Table 2, wherein the complex has a structure of:

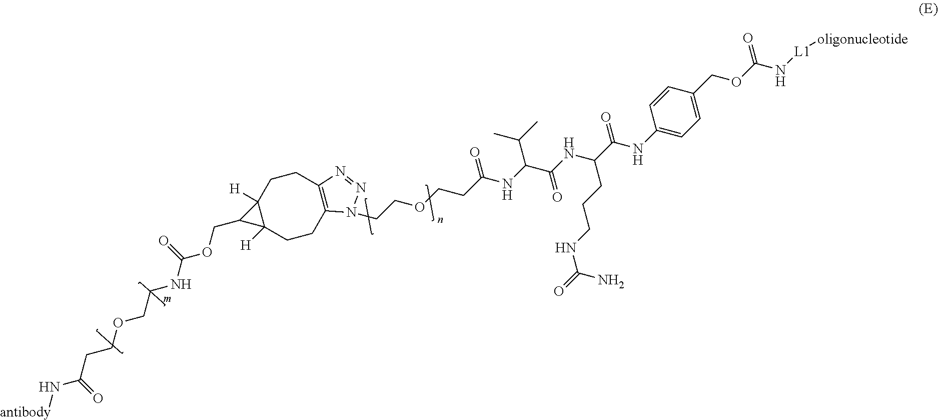

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, in any one of the examples of complexes described herein, the oligonucleotide comprises a region of complementarity to any one of the target gene described herein (e.g., DMPK, DMD, SMN, FXN, SOD1, C9orf72, ATXN2, FUS, LRRK2, SCNA, HTT, MSH3, TREM2, APOE, MAPT, APP, GYS1, PrP, VLA-4, UBE3A, GFAP, LSD, SCN9A, SCN1A, SCN2A, SCN8A, CLN3, GRIA1, and PCDH19). In some embodiments, in any one of the examples of complexes described herein, the oligonucleotide comprises a region of complementarity to any one of the target gene described herein (e.g., TOR1A, THAP1, ANO3, GNAL, KMT2B, GCH1, TH, SPR, TAF1, PRKRA, ATP1A3, SGCE, PNKD, PRRT2, SLC2A1, and ECHS1). In some embodiments, in any one of the examples of complexes described herein, the oligonucleotide comprises a region of complementarity to any one of the target genes described herein (e.g., PIKFYVE, SYF2, UNC13A, ATXN1, ATXN3, GRN, GRIN2A, TPP1, GLB1, ASM, ARSA, GALC, HEXA, HEXB, GBA, and MECP2). In some embodiments, the oligonucleotide comprises a region of complementarity to a target sequence as set forth in any one of SEQ ID NOs: 392-702. In some embodiments, the oligonucleotide comprises a region of complementarity to a target sequence as set forth in any one of SEQ ID NOs: 705-803. In some embodiments, the oligonucleotide comprising a region of complementarity to a target sequence as set forth in any one of SEQ ID NOs: 143-148, 167-169, 810-875, and 1059-1068. In some embodiments, the oligonucleotide comprises a nucleobase sequence of any one of the oligonucleotide molecular payloads provided in Tables 5-19 and optionally comprising any suitable modifications (e.g., modified nucleosides and/or internucleoside linkages). In some embodiments, the oligonucleotide comprises an oligonucleotide disclosed in any one of Tables 5-19.

In some embodiments, in any one of the examples of complexes described herein, L1 is

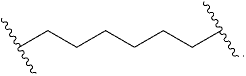 [SMILES: *CCCCCC*]

In some embodiments, the complex described herein comprises an anti-TfR1 Fab covalently linked to a 5′ end or a 3′ end of an oligonucleotide disclosed herein via a lysine in the anti-TfR1 antibody, wherein the anti-TfR1 Fab comprises a heavy chain and light chain of any one of the antibodies listed in Table 2, wherein the complex has a structure of:

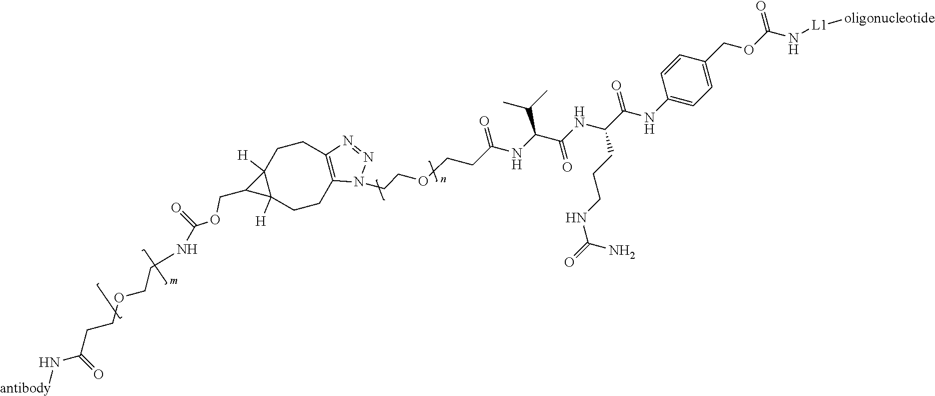

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, in any one of the examples of complexes described herein, L1 is:

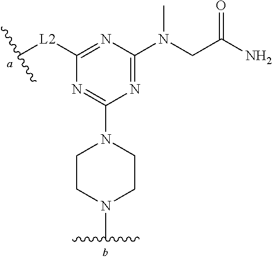

wherein L2 is

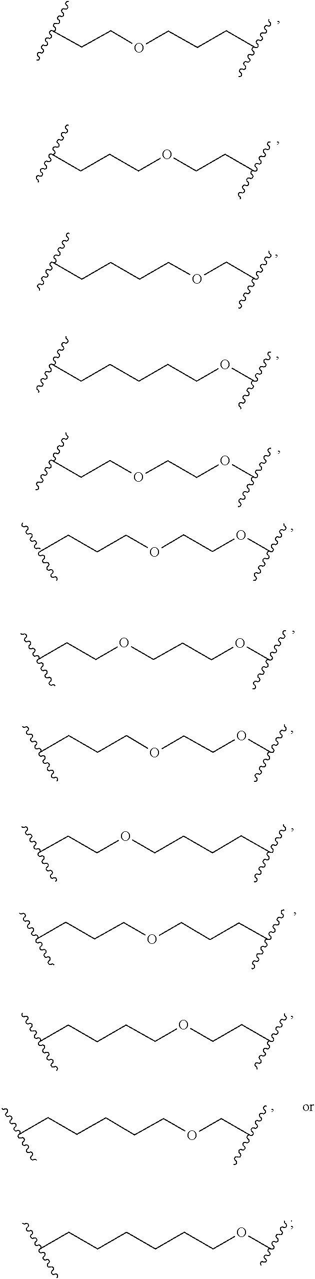

wherein a labels the site directly linked to the carbamate moiety of formulae (B), (D), (E), and (I); and b labels the site covalently linked (directly or via additional chemical moieties) to the oligonucleotide.

In some embodiments, in any one of the examples of complexes described herein, Li is:

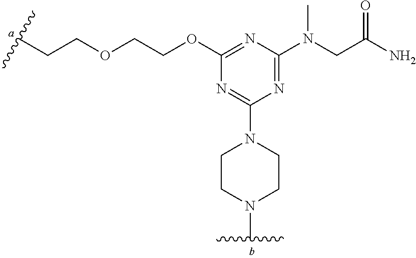 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is linked to a 5′ phosphate of the oligonucleotide. In some embodiments, the phosphate is a phosphodiester. In some embodiments, L1 is linked to a 5′ phosphorothioate of the oligonucleotide. In some embodiments, L1 is linked to a 5′ phosphonoamidate of the oligonucleotide. In some embodiments, L1 is linked via a phosphorodiamidate linkage to the 5′ end of the oligonucleotide.

In some embodiments, L1 is optional (e.g., need not be present).

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or (e.g., and) uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target CNS cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., CNS-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or (e.g., and) therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., molecular payload or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, intrathecal, intracerebrospinal intracerebroventricular, administration. In some embodiments, the route of administration is intranasal. Typically, the route of administration is intravenous, intrathecal, intracerebroventricular, or intranasal.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a CNS-targeting agent (e.g., an anti-TfR1 antibody) covalently linked to a molecular payload as described herein are effective in treating a subject having a CNS disease or disorder. In some embodiments, complexes comprise a molecular payload that is an oligonucleotide, polypeptide, small molecule, or gene therapy payload.

In some embodiments, a subject may be a human subject, a non-human primate subject, a rodent subject, or any suitable mammalian subject. In some embodiments, a subject may have a CNS disease or disorder.

In some embodiments, a subject has a mutation in a gene associated with a CNS disease or disorder. In some embodiments, the subject has been diagnosed with a CNS disease or disorder. In some embodiments, the subject has been determined to overexpress a gene associated with a CNS disease or disorder in one or more tissues (e.g., within the CNS, such as within the brain or a portion of the brain). In some embodiments, the subject has been determined to lack functional expression of a gene associated with a CNS disease or disorder in one or more tissues (e.g., within the CNS, such as within the brain or a portion of the brain). In some embodiments, the CNS disease or disorder is a neuromuscular disease or disorder (e.g., Duchenne muscular dystrophy, myotonic dystrophy, Friedreich's ataxia, or spinal muscular atrophy); amyotrophic lateral sclerosis; Parkinson's disease; Huntington's disease; Alzheimer's disease; epilepsy; a pain disorder; glycogen synthesis disorders; neurodegeneration; small fiber neuropathy; nociception-related phenotypes; Alexander disease; Angelman Syndrome; autism-spectrum disorders; retinitis pigmentosa; isolated macular dystrophy; and/or multiple sclerosis. In some embodiments, the CNS disease or disorder is essential tremor or hereditary dystonia. In some embodiments, the gene is DMPK, DMD, SMN, FXN, SOD1, C9orf72, ATXN2, FUS, LRRK2, SNCA, HTT, MSH3, TREM2, APOE, MAPT, APP, GYS1, PrP, VLA-4, GFAP, UBE3A, LSD, SCN9A, SCN1A, SCN2A, SCN8A, CLN3, GRIA1, or PCDH19. In some embodiments, the gene is TOR1A, THAP1, ANO3, GNAL, KMT2B, GCH1, TH, SPR, TAF1, PRKRA, ATP1A3, SGCE, PNKD, PRRT2, SLC2A1, or ECHS1. In some embodiments, the gene is PIKFYVE, SYF2, UNC13A, ATXN1, ATXN3, GRN, GRIN2A, TPP1, GLB1, ASM, ARSA, GALC, HEXA, HEXB, GBA, or MECP2.

An aspect of the disclosure includes methods involving administering to a subject an effective amount of a complex as described herein. In some embodiments, an effective amount of a pharmaceutical composition that comprises a complex comprising a CNS-targeting agent (e.g., an anti-TfR1 antibody) covalently linked to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, administration may be performed by intranasal, intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a CNS-targeting agent (e.g., an anti-TfR1 antibody) covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a CNS-targeting agent (e.g., an anti-TfR1 antibody) covalently linked to a molecular payload is administered at an effective concentration that confers therapeutic effect on a subject. Effective amounts vary, as recognized by those skilled in the art, depending on the severity of the disease or disorder, unique characteristics of the subject being treated, e.g., age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g., the half-life of the complex in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation or observation of symptoms associated with a CNS disease or disorder, such as symptoms associated with a neuromuscular disease or disorder (e.g., Duchenne muscular dystrophy, myotonic dystrophy, Friedreich's ataxia, or spinal muscular atrophy); amyotrophic lateral sclerosis; Parkinson's disease; Huntington's disease; Alzheimer's disease; epilepsy; a pain disorder; glycogen synthesis disorders; neurodegeneration; small fiber neuropathy; nociception-related phenotypes; Alexander disease; Angelman Syndrome; autism-spectrum disorders; retinitis pigmentosa; isolated macular dystrophy; and/or multiple sclerosis.

In some embodiments, a pharmaceutical composition that comprises a complex comprising a CNS-targeting agent (e.g., an anti-TfR1 antibody) covalently linked to a molecular payload described herein is administered to a subject at an effective concentration sufficient to modulate activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### EXAMPLES

### Example 1. CNS Delivery of a DMPK-Targeting Oligonucleotide (ASO) in a Complex with an Anti-TfR1 Antibody

Complexes comprising an anti-TfR1 Fab covalently linked to a gapmer antisense oligonucleotide (ASO) targeting mouse Dmpk were tested in a mouse that expresses both human TfR1 (hTfR1) and one copy of a mutant human DMPK transgene that harbors expanded CTG repeats (hTfR1/DMSXL mice). One anti-TfR1 Fab used (“Anti-TfR1 Fab1”) has the VH and VL amino acid sequences provided in Table 2. The other anti-TfR1 Fab used (“Anti-TfR1 Fab2”) has VH and VL with distinct amino acid sequences. The anti-TfR1 Fabs were covalently linked to the ASO via a cleavable linker comprising the structure of Formula (I). Mice were administered either vehicle control (PBS; “Vehicle”), 10 mg/kg ASO not covalently linked to an antibody (“Naked ASO”), 10 mg/kg ASO-equivalent of anti-TfR1 Fab 1-ASO complexes (“Anti-TfR1 Fab1-ASO Complex”), or 10 mg/kg ASO-equivalent of anti-TfR1 Fab 2-ASO complexes (“Anti-TfR1 Fab2-ASO Complex”), on days 0 and 7 via intravenous injection. Mice were sacrificed at day 14 (one week following the second administration of PBS, naked ASO, or complexes), and brain tissue was collected.

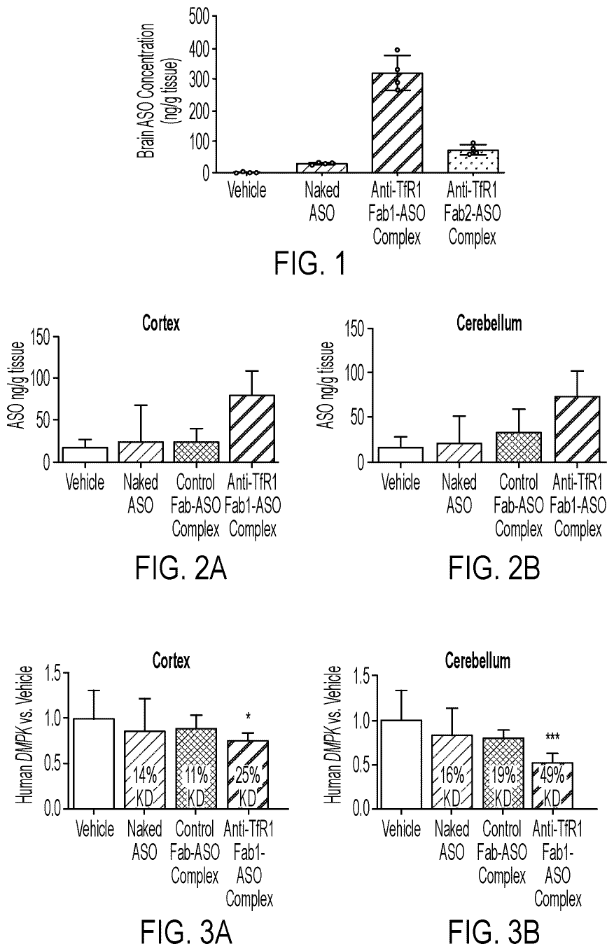

ASO content in the brain tissue was measured by hybridization ELISA (Burki et al., Nucleic Acid Ther. 2015 October; 25(5):275-84, incorporated herein by reference). FIG. 1 shows the amount of ASO measured in brain tissue of mice administered PBS, Naked ASO, or Anti-TfR1 Fab1-ASO Complexes. There was little ASO detected in the brain tissue of mice administered naked ASO or anti-TfR1 Fab2-ASO complexes, whereas high levels of ASO were detected in the brain tissue of mice administered anti-TfR1 Fab1-ASO complexes.

These results demonstrate that complexes comprising an anti-TfR1 antibody having the VH and VL sequences provided in Table 2 (e.g., anti-TfR1 Fab1) are capable of delivering a molecular payload (e.g., an oligonucleotide) into the brain following intravenous administration. The results also demonstrate that complexes comprising other anti-TfR1 antibodies (i.e., not having the VH and VL sequences provided in Table 2) may not be capable of delivering a payload into the brain following intravenous administration.

### Example 2. Knockdown Activity of a DMPK-Targeting Oligonucleotide (ASO) in hTfR1/DMSXL Homozygous Mice

Complexes comprising an anti-TfR1 Fab covalently linked to a human DMPK-targeting oligonucleotide having an oligonucleotide structure of +C*+C*oA*oG*xdC*dG*dC*dC*dC*dA*dC*dC*oA*oG*+U*+C (SEQ ID NO: 703) (in which +N represents an LNA nucleoside, oN represents a 2′-MOE modified ribonucleoside, dN represents a 2′-deoxyribonucleoside, xdC represents a 5-methyl-deoxycytidine, +C represents a 5-methyl-2′-4′-bicyclic-cytidine (2′-4′ methylene bridge), +U represents a 5-methyl-2′-4′-bicyclic-uridine (2′-4′ methylene bridge), and * represents a phosphorothioate internucleoside linkage) were tested in a mouse that expresses both human TfR1 and two copies of a mutant human DMPK transgene that harbors expanded CTG repeats (hTfR1/DMSXL mice). The anti-TfR1 Fab used (“Anti-TfR1 Fab1”) has the VH and VL amino acid sequences provided in Table 2. Complexes comprising the same ASO and a control antibody (a Fab which recognizes an HIV antigen and is isotype-matched with anti-TfR1 Fab1, “control Fab”) were also tested as a control. The Fabs were covalently linked to the ASO via a cleavable linker comprising the structure of Formula (I). Mice were administered either vehicle control (25 mM Tris, 10% sucrose, pH 7.4; “Vehicle”), 10 mg/kg ASO not covalently linked to an antibody (“Naked ASO”), 10 mg/kg ASO-equivalent of control Fab-ASO complexes (“Control Fab-ASO Complex”), or 10 mg/kg ASO-equivalent of anti-TfR1 Fab1-ASO complexes (“Anti-TfR1 Fab1-ASO Complex”), on days 0 and 7 via intravenous injection. Mice were sacrificed at day 35 (four weeks following the second administration of vehicle, naked ASO, or complexes), and brain tissue was collected.

ASO content in the brain tissue was measured by hybridization ELISA (Burki et al., Nucleic Acid Ther. 2015 October; 25(5):275-84, incorporated herein by reference). FIGS. 2A and 2B show the amount of ASO measured in the cortex (FIG. 2A) and cerebellum (FIG. 2B) of treated mice. There was little ASO detected in the cortex or cerebellum of mice administered naked ASO or control Fab-ASO complexes, whereas higher levels of ASO were detected in the cortex and cerebellum of mice administered anti-TfR1 Fab1-ASO complexes.

RNA was also extracted from collected brain tissues, and reverse transcription-quantitative polymerase chain reaction (qRT-PCR) of the RNA samples was performed to measure human DMPK and mouse Gapdh (glyceraldehyde-3-phosphate dehydrogenase) as an internal control.

Human mutant DMPK expression, normalized to vehicle-treated control mice, in the cortex and cerebellum are shown in FIGS. 3A and 3B, respectively. Naked ASO and control Fab-ASO complexes had no significant effect on human mutant DMPK expression in the cortex or cerebellum of mice relative to vehicle control-treated mice. Anti-TfR1 Fab1-ASO complexes significantly reduced human mutant DMPK expression, by 25% in the cortex and by 49% in the cerebellum. This demonstrates that delivery of the ASO to the brain resulted in reduced expression of the DMPK target.

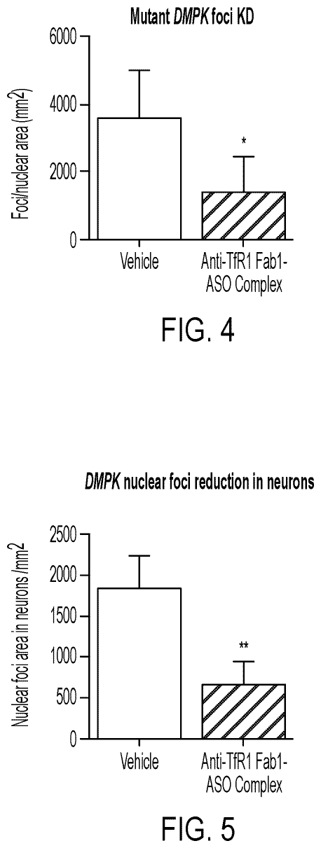

Brain tissue sections were also prepared and imaged for human DMPK foci. Samples from vehicle-treated control mice and anti-TfR1 Fab1-ASO complex-treated mice were prepared and imaged following fluorescence in situ hybridization (FISH) for mutant human DMPK. FISH visualization in the cerebellum and cortex demonstrated decreases in toxic mutant nuclear human DMPK foci in both brain regions in mice administered the anti-TfR1 Fab1-ASO complexes relative to those administered the vehicle control. In cerebellum, substantial nuclear human DMPK foci were observed in cells of the molecular and Purkinje layers (including in Bergmann glia in the Purkinje layer) in vehicle control mice, and fewer and smaller nuclear foci were observed in corresponding cells in mice administered the complexes. In cortex, substantial nuclear human DMPK foci were observed in cells of vehicle control mice, and fewer and smaller nuclear foci were observed in corresponding cells in mice administered the complexes. Quantification (FIG. 4) of mutant nuclear human DMPK foci in the cerebellum in FISH microscopy images showed significant decreases in toxic mutant nuclear human DMPK foci. The quantification of human mutant DMPK foci in the cerebellum showed a significant decrease of about 62% in the cerebellum of anti-TfR1 Fab1-ASO complex-treated mice relative to vehicle-treated mice. These results demonstrate that the anti-TfR1 Fab1-ASO complexes are able to target the nuclear RNA species. The results further demonstrate that the complexes are able to reduce mutant DMPK RNA foci in cells of the brain parenchyma, indicating that the complexes facilitate delivery of the ASO across the blood-brain barrier.

These results demonstrate that complexes comprising an anti-TfR1 antibody having the VH and VL sequences provided in Table 2 (e.g., anti-TfR1 Fab1) are capable of delivering a molecular payload (e.g., a molecular payload disclosed herein, such as an oligonucleotide, polypeptide, small molecule, or gene therapy payload) into the cortex and cerebellum following intravenous administration, and that once delivered, the molecular payload is able to exert its function (e.g., suppression of expression of its target gene including within the nucleus of target CNS cells).

### Example 3. Knockdown of Toxic DMPK Foci in Cortical Neurons of hTfR1/DMSXL Mice

Complexes comprising an anti-TfR1 Fab covalently linked to a human DMPK-targeting oligonucleotide having an oligonucleotide structure of +C*+C*oA*oG*xdC*dG*dC*dC*dC*dA*dC*dC*oA*oG*+U*+C (SEQ ID NO: 703) (in which +N represents an LNA nucleoside, oN represents a 2′-MOE modified ribonucleoside, dN represents a 2′-deoxyribonucleoside, xdC represents a 5-methyl-deoxycytidine, +C represents a 5-methyl-2′-4′-bicyclic-cytidine (2′-4′ methylene bridge), +U represents a 5-methyl-2′-4′-bicyclic-uridine (2′-4′ methylene bridge), and * represents a phosphorothioate internucleoside linkage) were tested in a mouse that expresses both human TfR1 and two copies of a mutant human DMPK transgene that harbors expanded CTG repeats (hTfR1/DMSXL mice). The anti-TfR1 Fab used (“Anti-TfR1 Fab1”) has the VH and VL amino acid sequences provided in Table 2. The Fab was covalently linked to the ASO via a cleavable linker comprising the structure of Formula (I). Mice were administered either vehicle control (25 mM Tris, 10% sucrose, pH 7.4; “Vehicle”) or 10 mg/kg ASO-equivalent of anti-TfR1 Fab1-ASO complexes (“Anti-TfR1 Fab1-ASO Complex”), on days 0 and 7 via intravenous injection. Mice were sacrificed at day 35 (four weeks following the second administration of vehicle or complexes), and brain tissue was collected.

Brain tissue sections were prepared and imaged for human DMPK foci. Samples from vehicle-treated control mice and anti-TfR1 Fab1-ASO complex-treated mice were prepared and imaged following fluorescence in situ hybridization (FISH) for mutant human DMPK. FISH visualization in the cortex demonstrated decreases in toxic mutant nuclear human DMPK foci in mice administered the anti-TfR1 Fab1-ASO complexes relative to those administered the vehicle control. Substantial nuclear human DMPK foci were observed in cells of vehicle control mice, and fewer and smaller nuclear foci were observed in corresponding cells in mice administered the complexes. Quantification (FIG. 5) of mutant nuclear human DMPK foci in the cerebellum in FISH microscopy images showed significant decreases in toxic mutant nuclear human DMPK foci. The quantification of human mutant DMPK foci in the cerebellum showed a significant decrease of about 62% in the cerebellum of anti-TfR1 Fab1-ASO complex-treated mice relative to vehicle-treated mice. These results demonstrate that the anti-TfR1 Fab1-ASO complexes are able to target the nuclear RNA species. The results further demonstrate that the complexes are able to reduce mutant DMPK RNA foci in cells of the brain parenchyma, indicating that the complexes facilitate delivery of the ASO across the blood-brain barrier.

To assess the effect of anti-TfR1 Fab1-ASO complexes on specific CNS parenchyma cell types such as neurons, brain tissue sections were co-stained for mutant human DMPK by fluorescence in situ hybridization (FISH) and for neuron marker NeuN by immunofluorescence. Sections were counterstained with DAPI (4′,6-diamidino-2-phenylindole) to label nuclei. Fluorescence images were acquired by confocal microscopy, which showed substantial nuclear human DMPK foci in NeuN-stained neurons of vehicle control mice, and fewer and smaller nuclear foci in corresponding cells in mice administered the complexes. Quantitative analysis showed a 64% reduction of mutant nuclear human DMPK foci area in NeuN-stained neurons in mice administered the anti-TfR1 Fab1-ASO complexes relative to mice administered the vehicle control (FIG. 5).

These results demonstrate that complexes comprising an anti-TfR1 antibody having the VH and VL sequences provided in Table 2 (e.g., anti-TfR1 Fab1) are capable of delivering a molecular payload (e.g., a molecular payload disclosed herein, such as an oligonucleotide, polypeptide, small molecule, or gene therapy payload) into the cortex following intravenous administration, and that once delivered, the molecular payload is able to exert its function (e.g., suppression of expression of its target gene including within the nucleus of target CNS cells).

### Example 4. Knockdown of Human DMPK in the Brain with a Single Dose of Fab-ASO Complexes in hTfR1/DMSXL Hemizygous Mice

Complexes comprising an anti-TfR1 Fab covalently linked to human DMPK-targeting antisense oligonucleotides were prepared, comprising ASO1 having an oligonucleotide structure of +C*+C*oA*oG*xdC*dG*dC*dC*dC*dA*dC*dC*oA*oG*+U*+C (SEQ ID NO: 703), ASO2 having an oligonucleotide structure of +G*+C*oA*oC*dG*dT*dG*dT*dG*dG*xdC*dT*oC*oA*+A*+G (SEQ ID NO:804), ASO3 having an oligonucleotide structure of +A*+C*oC*oA*dA*xdC*dA*xdC*dG*dT*xdC*xdC*oC*oU*+C*+U (SEQ ID NO:805), or ASO4 having an oligonucleotide structure of +C*+C*oC*oG*dG*dA*dT*xdC*dA*xdC*dA*dG*oG*oA*+C*+U (SEQ ID NO:806) (in each of which +N represents an LNA nucleoside, oN represents a 2′-MOE modified ribonucleoside, dN represents a 2′-deoxyribonucleoside, xdC represents a 5-methyl-deoxycytidine, oC represents a 5-methyl-2′-MOE-cytidine, +C represents a 5-methyl-2′-4′-bicyclic-cytidine (2′-4′ methylene bridge), +U represents a 5-methyl-2′-4′-bicyclic-uridine (2′-4′ methylene bridge), and * represents a phosphorothioate internucleoside linkage). The anti-TfR1 Fab used (“anti-TfR1 Fab1”) has the VH and VL amino acid sequences provided in Table 2. Anti-TfR1 Fab1 was covalently linked to each ASO via a cleavable linker comprising the structure of Formula (I) to form the complexes. The complexes were tested in a mouse that expresses both human TfR1 and one copy of a mutant human DMPK transgene that harbors expanded CTG repeats (hTfR1/DMSXL mice). Mice (n=3-5 per group) were administered either vehicle control (25 mM Tris, 10% sucrose, pH 7.4; “Vehicle”), or 10 mg/kg ASO-equivalent of anti-TfR1 Fab1-ASO complexes, on day 0 via intravenous injection. Mice were sacrificed at day 56 (eight weeks following the administration of vehicle or complexes), and brain tissue was collected.

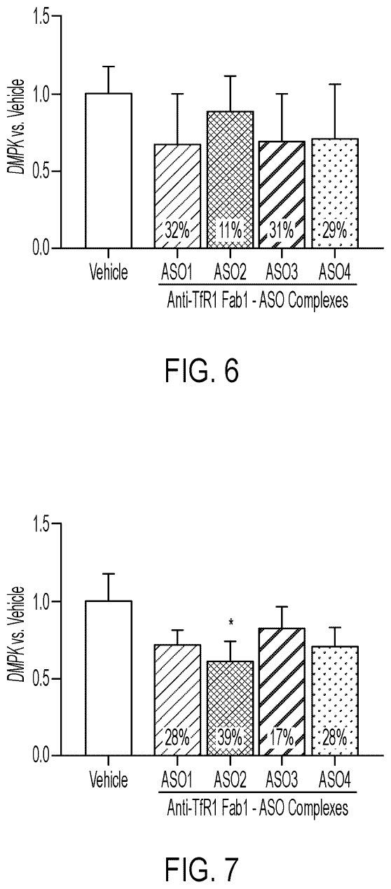

Human mutant DMPK expression in the brain, normalized to vehicle-treated control mice, was measured, shown in FIG. 6. The anti-TfR1 Fab1-ASO complexes each reduced human mutant DMPK expression in the brain relative to vehicle-treated mice: anti-TfR1 Fab1-ASO1 complexes reduced expression by 32%; anti-TfR1 Fab1-ASO2 complexes reduced expression by 11%; anti-TfR1 Fab1-ASO3 complexes reduced expression by 31%; and anti-TfR1 Fab1-ASO4 complexes reduced expression by 29%.

These results demonstrate that complexes comprising an anti-TfR1 antibody having the VH and VL sequences provided in Table 2 (e.g., anti-TfR1 Fab1) are capable of delivering a molecular payload (e.g., a molecular payload disclosed herein, such as an oligonucleotide, polypeptide, small molecule, or gene therapy payload) into the brain following intravenous administration, and that once delivered, the molecular payload is able to exert its function (e.g., suppression of expression of its target gene).

### Example 5. Knockdown of Human DMPK in the Brain with Repeat Doses of Fab-ASO Complexes in hTfR1/DMSXL Hemizygous Mice

Complexes comprising an anti-TfR1 Fab covalently linked to human DMPK-targeting antisense oligonucleotides were prepared, comprising ASO1 having an oligonucleotide structure of +C*+C*oA*oG*xdC*dG*dC*dC*dC*dA*dC*dC*oA*oG*+U*+C (SEQ ID NO: 703), ASO2 having an oligonucleotide structure of +G*+C*oA*oC*dG*dT*dG*dT*dG*dG*xdC*dT*oC*oA*+A*+G (SEQ ID NO:804), ASO3 having an oligonucleotide structure of +A*+C*oC*oA*dA*xdC*dA*xdC*dG*dT*xdC*xdC*oC*oU*+C*+U (SEQ ID NO:805), or ASO4 having an oligonucleotide structure of +C*+C*oC*oG*dG*dA*dT*xdC*dA*xdC*dA*dG*oG*oA*+C*+U (SEQ ID NO:806) (in each of which +N represents an LNA nucleoside, oN represents a 2′-MOE modified ribonucleoside, dN represents a 2′-deoxyribonucleoside, xdC represents a 5-methyl-deoxycytidine, oC represents a 5-methyl-2′-MOE-cytidine, +C represents a 5-methyl-2′-4′-bicyclic-cytidine (2′-4′ methylene bridge), +U represents a 5-methyl-2′-4′-bicyclic-uridine (2′-4′ methylene bridge), and * represents a phosphorothioate internucleoside linkage). The anti-TfR1 Fab used (“anti-TfR1 Fab1”) has the VH and VL amino acid sequences provided in Table 2. Anti-TfR1 Fab1 was covalently linked to each ASO via a cleavable linker comprising the structure of Formula (I) to form the complexes. The complexes were tested in a mouse that expresses both human TfR1 and one copy of a mutant human DMPK transgene that harbors expanded CTG repeats (hTfR1/DMSXL mice). Mice were administered either vehicle control (25 mM Tris, 10% sucrose, pH 7.4; “Vehicle”), or 2.5 mg/kg ASO-equivalent of anti-TfR1 Fab1-ASO complexes, on days 0 and 28 via intravenous injection. Mice were sacrificed at day 56 (four weeks following the second administration of vehicle or complexes), and brain tissue was collected.

Human mutant DMPK expression in the brain, normalized to vehicle-treated control mice, was measured, shown in FIG. 7. The anti-TfR1 Fab1-ASO complexes each reduced human mutant DMPK expression in the brain relative to vehicle-treated mice: anti-TfR1 Fab1-ASO1 complexes reduced expression by 28%; anti-TfR1 Fab1-ASO2 complexes reduced expression by 39%; anti-TfR1 Fab1-ASO3 complexes reduced expression by 17%; and anti-TfR1 Fab1-ASO4 complexes reduced expression by 28%.

These results demonstrate that complexes comprising an anti-TfR1 antibody having the VH and VL sequences provided in Table 2 (e.g., anti-TfR1 Fab1) are capable of delivering a molecular payload (e.g., a molecular payload disclosed herein, such as an oligonucleotide, polypeptide, small molecule, or gene therapy payload) into the brain following intravenous administration, and that once delivered, the molecular payload is able to exert its function (e.g., suppression of expression of its target gene).

### Example 6. Delivery of Anti-TfR1 Fab1-ASO in the Brain of Nonhuman Primates after a Single Dose Administration

Complexes containing an anti-TfR1 Fab covalently linked to a DMPK-targeting antisense oligonucleotide having an oligonucleotide structure of +C*+C*oA*oG*xdC*dG*dC*dC*dC*dA*dC*dC*oA*oG*+U*+C (SEQ ID NO: 703) (in which +N represents an LNA nucleoside, oN represents a 2′-MOE modified ribonucleoside, dN represents a 2′-deoxyribonucleoside, xdC represents a 5-methyl-deoxycytidine, +C represents a 5-methyl-2′-4′-bicyclic-cytidine (2′-4′ methylene bridge), +U represents a 5-methyl-2′-4′-bicyclic-uridine (2′-4′ methylene bridge), and * represents a phosphorothioate internucleoside linkage) were tested in nonhuman primates. The anti-TfR1 Fab used (“anti-TfR1 Fab1”) has the VH and VL amino acid sequences provided in Table 2. The anti-TfR1 Fab was covalently linked the ASO via a cleavable linker comprising the structure of Formula (I) to form the complexes.

Cynomolgus monkeys were administered 10 mg/kg ASO not covalently linked to an antibody (“Naked ASO”) either as a single intravenous (IV) injection into a peripheral vein or a single intrathecal lumbar injection, or 10 mg/kg ASO-equivalent of anti-TfR1 Fab1-ASO complexes as a single IV injection into a peripheral vein. Animals were sacrificed 2, 24, or 72 hours following the administration of the ASO or the complexes. Brain tissue was collected for analysis.

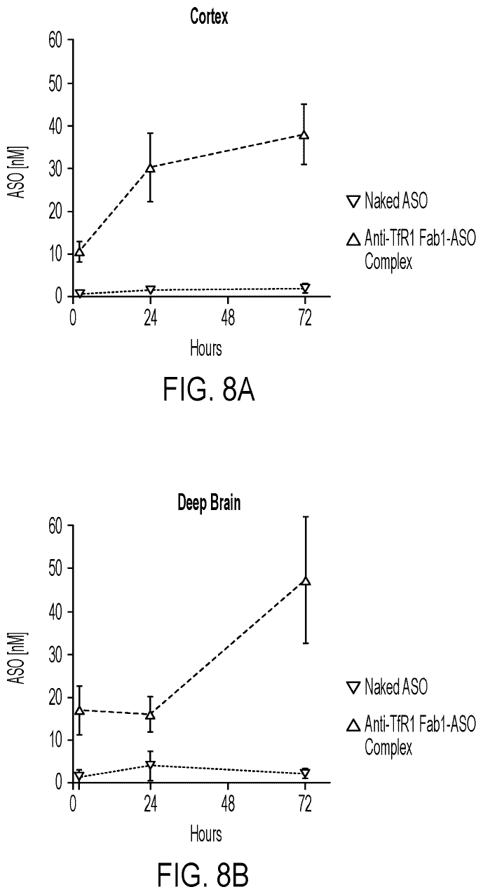

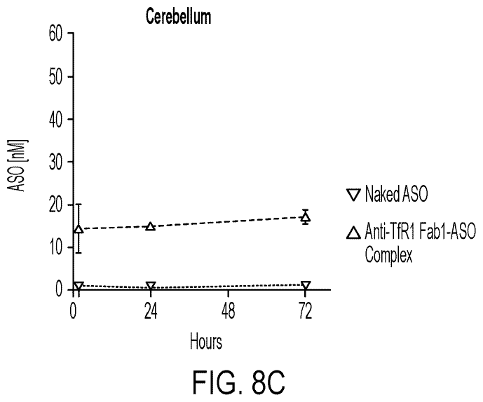

ASO content in the brain tissue of IV administered animals was measured by hybridization-based ELISA. FIGS. 8A-8C show the amount of ASO measured in the cortex (FIG. 8A), deep brain (FIG. 8B) and cerebellum (FIG. 8C) over time. There was little ASO detected in the brain of monkeys administered naked ASO, whereas higher levels of ASO were detected in the cortex, deep brain and cerebellum of monkeys administered anti-TfR1 Fab1-ASO complex. ASO quantified in the cortex was approximately 10 nM at the 2 hour timepoint, and was approximately 38 nM at the 72 hour timepoint. ASO quantified in the deep brain was approximately 17 nM at the 2 and 24 hour timepoints, and was approximately 45 nM at the 72 hour timepoint. ASO quantified in the cerebellum was approximately 15 nM at all timepoints tested.

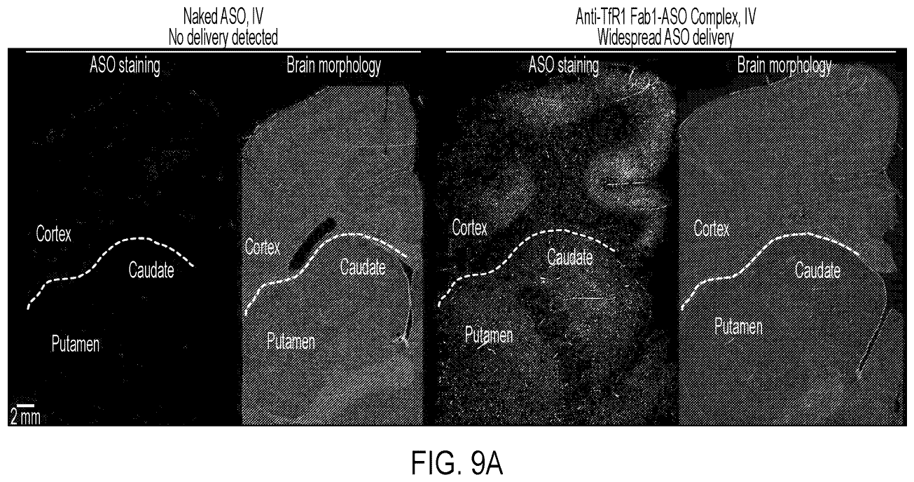

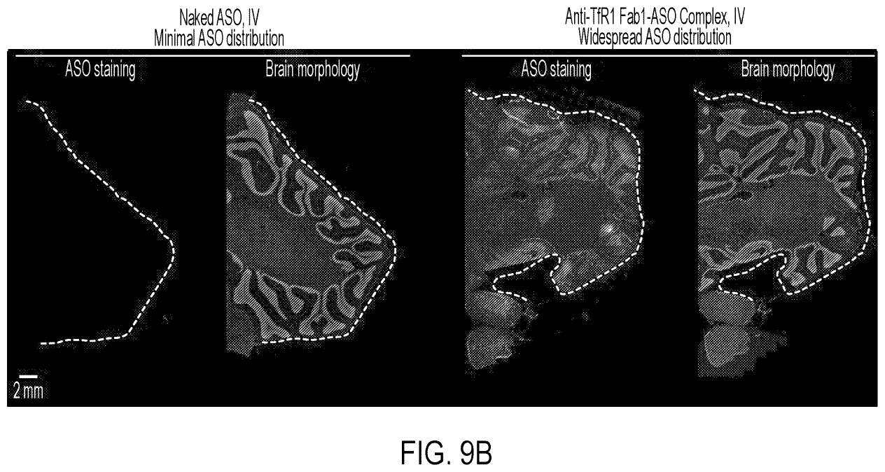

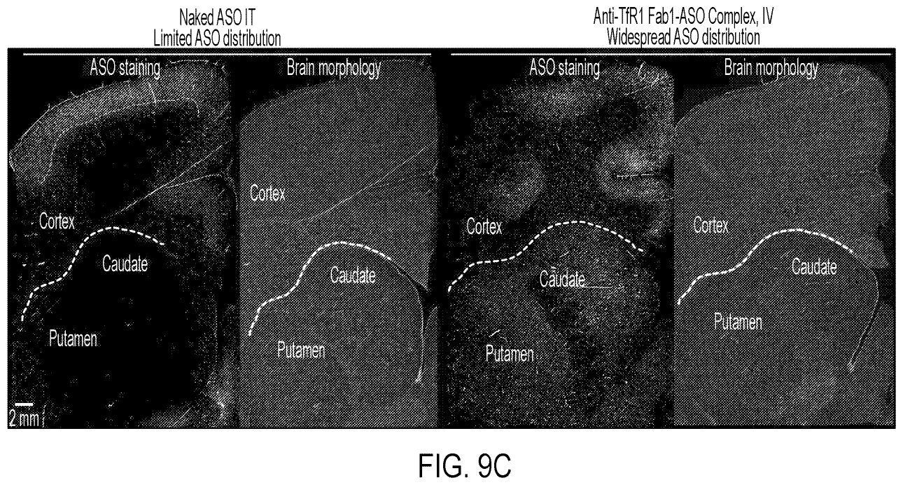

ASO distribution and cellular localization in the brain following IV administration of the complexes or IT or IV administration of naked ASO to the monkeys was visualized by in situ hybridization. FIGS. 9A and 9B show distribution of ASO in the brain following IV administration of naked ASO or anti-TfR1 Fab1-ASO complexes. FIG. 9C shows distribution of ASO in the brain following IT administration of naked ASO or IV administration of anti-TfR1 Fab1-ASO complexes. The results show superior delivery to cortex and deep brain (FIG. 9A) and cerebellum (FIG. 9B) of the ASO following IV administration of the complexes relative to IV administration of naked ASO. Caudate nucleus and putamen regions represent the deep brain. The results also show broader delivery in cortex and deep brain of the ASO following IV administration of the complexes relative to IT administration of naked ASO (FIG. 9C). No ASO was detected in brain tissues following IV administration of naked ASO (FIGS. 9A and 9B). ASO distribution was confined within superficial layers of the cortex following IT administration of naked ASO (FIG. 9C).

IV dosing of the complexes delivered up to ~25 to 45-fold higher concentration of ASO compared to IV administration of the naked ASO to cortex and deep brain regions as well as cerebellum. Moreover, ASO detection by in situ hybridization in brain sections demonstrated that dosing with the complexes resulted in widespread delivery of the ASO including the cortex and deep brain regions. In contrast, naked ASO administered IV was not detected, and naked ASO injected IT remained confined within the most superficial layers of the cerebrum and cerebellar cortex.

These data indicate that complexes comprising an anti-TfR1 antibody having the VH and VL sequences provided in Table 2 (e.g., anti-TfR1 Fab1) can deliver a molecular payload (e.g., a molecular payload disclosed herein, such as an oligonucleotide, polypeptide, small molecule, or gene therapy payload) into various regions of the brain following systemic (e.g., IV) administration, and that complexes achieve more widespread delivery of such cargoes than is possible by direct IT administration. This demonstrates that the complexes have the potential to affect CNS diseases and disorders, such as CNS manifestations of DM1.

### Example 7. Knockdown Activity of DMPK-Targeting Fab-ASO Complexes in hTfR1/DMSXL Homozygous Mice

Anti-TfR1 Fab1-ASO complexes (“Anti-TfR1 Fab1-ASO Complexes”) comprising an anti-TfR1 Fab covalently linked to human DMPK-targeting antisense oligonucleotides were prepared, comprising ASO2 having an oligonucleotide structure of +G*+C*oA*oC*dG*dT*dG*dT*dG*dG*xdC*dT*oC*oA*+A*+G (SEQ ID NO:804), or comprising ASO4 having an oligonucleotide structure of +C*+C*oC*oG*dG*dA*dT*xdC*dA*xdC*dA*dG*oG*oA*+C*+U (SEQ ID NO:806) (in both of which +N represents an LNA nucleoside, oN represents a 2′-MOE modified ribonucleoside, dN represents a 2′-deoxyribonucleoside, xdC represents a 5-methyl-deoxycytidine, oC represents a 5-methyl-2′-MOE-cytidine, +C represents a 5-methyl-2′-4′-bicyclic-cytidine (2′-4′ methylene bridge), +U represents a 5-methyl-2′-4′-bicyclic-uridine (2′-4′ methylene bridge), and * represents a phosphorothioate internucleoside linkage). The anti-TfR1 Fab used (“anti-TfR1 Fab1”) has the VH and VL amino acid sequences provided in Table 2. Anti-TfR1 Fab1 was covalently linked to each ASO via a cleavable linker comprising the structure of Formula (I) to form the Complexes.

The Complexes were tested in a mouse that expresses both human TfR1 and two copies of a mutant human DMPK transgene that harbors expanded CTG repeats (hTfR1/DMSXL homozygous mice). Mice (n=6-7 per group) were administered either vehicle control (25 mM Tris, 10% Sucrose, pH 7.4; “Vehicle”) or ASO-equivalent doses of 5 mg/kg or 10 mg/kg of Anti-TfR1 Fab1-ASO Complexes, on days 0 and 28 via intravenous injection (“2×5” and “2×10”, respectively). Mice were sacrificed on day 56 (four weeks following the second administration of Vehicle, or Anti-TfR1 Fab1-ASO Complexes), and CNS tissues, including brain and spinal cord, were collected.

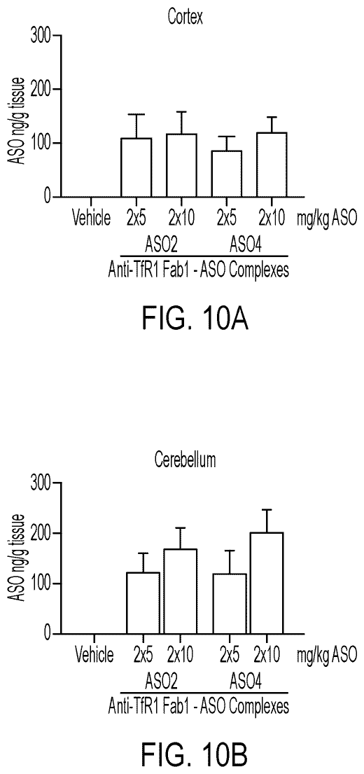

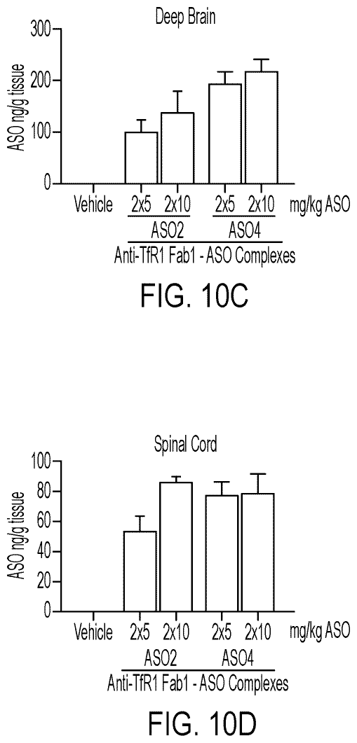

ASO content in the brain tissue was measured by hybridization ELISA (Burki et al., Nucleic Acid Ther. 2015 October; 25(5):275-84, incorporated herein by reference). FIGS. 10A-10D. show the amount of ASO measured in the cortex (FIG. 10A), cerebellum (FIG. 10B), deep brain regions (FIG. 10C), and spinal cord (FIG. 10D) of treated mice. Two dosing levels of both Anti-TfR1 Fab1-ASO Complexes demonstrated substantial levels of ASO detectable across CNS tissues.

RNA was also extracted from collected CNS tissues, and reverse transcription-quantitative polymerase chain reaction (qRT-PCR) of the RNA samples was performed to measure human DMPK and mouse Gapdh (Glyceraldehyde-3-Phosphate Dehydrogenase) as an internal control.

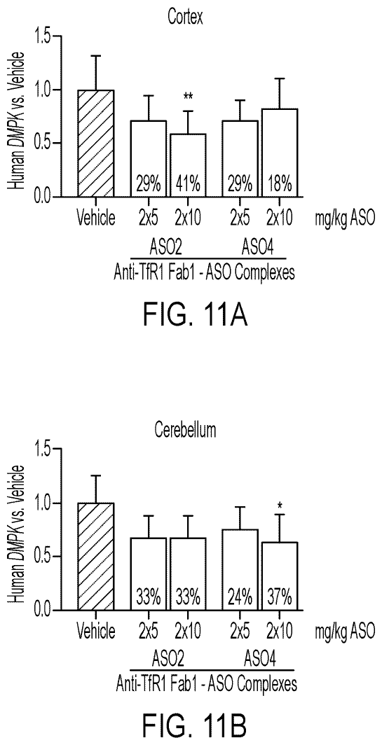

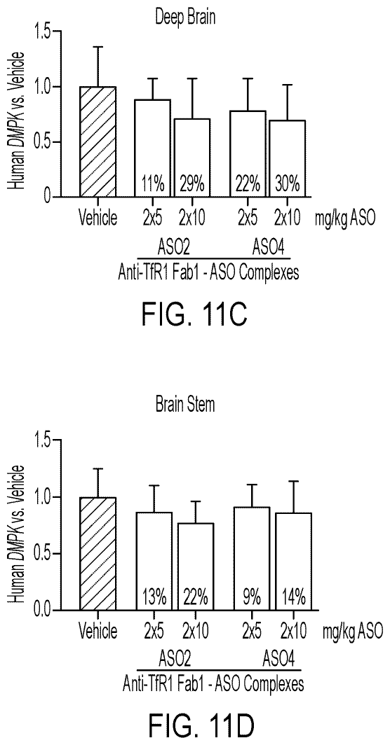

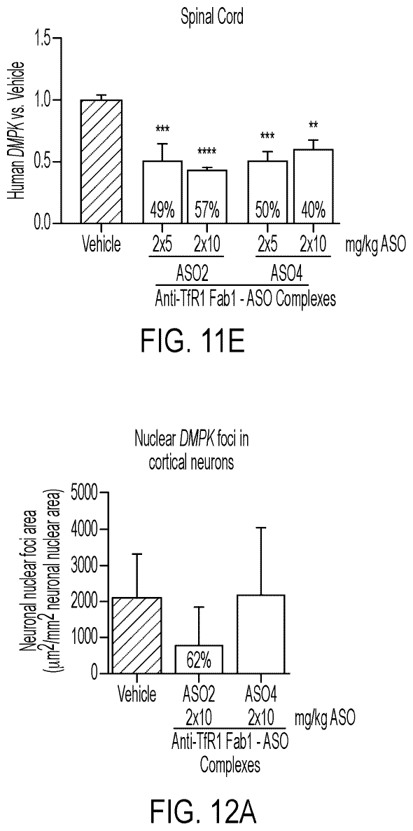

Human mutant DMPK expression, normalized to Vehicle-treated control mice, in the brain regions (cortex, cerebellum, deep brain, and brain stem) is shown in FIGS. 11A-11D, respectively. Administration of Anti-TfR1 Fab1-ASO Complexes resulted in robust human mutant DMPK knockdown in cortex (29% and 41% knockdown with ASO2 and 29% and 18% knockdown with ASO4, at 2×5 and 2×10 doses, respectively; FIG. 11A) and cerebellum regions (33% and 33% knockdown with ASO2 and 24% and 37% knockdown with ASO4, at 2×5 and 2×10 doses, respectively; FIG. 11B), and moderate DMPK knockdown in deep brain (11% and 29% knockdown with ASO2 and 22% and 30% knockdown with ASO4, at 2×5 and 2×10 doses, respectively; FIG. 11C) and brain stem regions (13% and 22% knockdown with ASO2 and 9% and 14% knockdown with ASO4, at 2×5 and 2×10 doses, respectively; FIG. 11D).

Human mutant DMPK expression, normalized to Vehicle-treated control mice, in the spinal cord is shown in FIG. 11E. Administration of Anti-TfR1 Fab1-ASO Complexes at both dosing levels resulted in robust human mutant DMPK knockdown in spinal cord (49% and 57% knockdown with ASO2 and 50% and 40% knockdown with ASO4, at 2×5 and 2×10 doses, respectively).

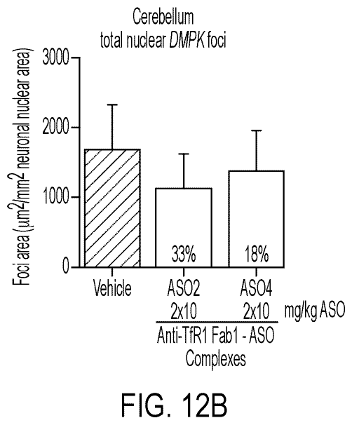

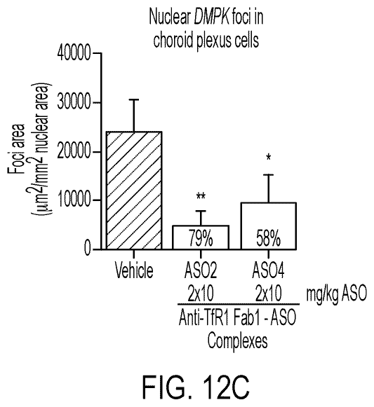

Brain tissue sections were also prepared and imaged for human DMPK foci. Samples from Vehicle-treated control mice and 2×10 mg/kg Anti-TfR1 Fab1-ASO Complex-treated mice were prepared and imaged following immunofluorescence (IF) staining for neurons (NeuN), and fluorescence in situ hybridization (FISH) for mutant human DMPK. FISH quantification of mutant nuclear human DMPK foci in cortical neurons, cerebellum and choroid plexus cells are shown in FIGS. 12A, 12B, and 12C, respectively. In cortex, quantification of human mutant nuclear DMPK foci showed 62% reduction in neurons of mice treated with anti-TfR1 Fab1-ASO2 complexes relative to Vehicle-treated control mice (FIG. 12A). The quantification of human mutant nuclear DMPK foci showed 33% (ASO2 complexes) and 18% (ASO4 complexes) reduction in the cerebellum, and 79% (ASO2 complexes) and 58% (ASO4 complexes) reduction in the choroid plexus cells of mice treated with Anti-TfR1 Fab1-ASO Complexes relative to Vehicle-treated control mice (FIGS. 12B and 12C, respectively). These results demonstrate that Anti-TfR1 Fab1-ASO Complexes are able to target the nuclear RNA species in multiple brain regions, and reduce mutant DMPK RNA foci in cells of the CNS, including brain parenchymal cells and choroid plexus cells.

These results demonstrate that Anti-TfR1 Fab1-ASO Complexes can deliver to various regions of the CNS, including different regions of the brain and spinal cord, following intravenous administration, and show that the Complexes are highly potent in the suppression of human mutant DMPK expression within the nuclei of multiple CNS cell types.

### ADDITIONAL EMBODIMENTS

1. A complex comprising an anti-TfR1 antibody covalently linked to a molecular payload for treating a central nervous system (CNS) disease or disorder, wherein the anti-TfR1 antibody comprises:
(i) a heavy chain complementarity determining region 1 (CDR-H1) of SEQ ID NO: 1, a heavy chain complementarity determining region 2 (CDR-H2) of SEQ ID NO: 2, a heavy chain complementarity determining region 3 (CDR-H3) of SEQ ID NO: 3, a light chain complementarity determining region 1 (CDR-L1) of SEQ ID NO: 4, a light chain complementarity determining region 2 (CDR-L2) of SEQ ID NO: 5, and a light chain complementarity determining region 3 (CDR-L3) of SEQ ID NO: 6;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6; or
(iii) a CDR-H1 of SEQ ID NO: 12, a CDR-H2 of SEQ ID NO: 13, a CDR-H3 of SEQ ID NO: 14, a CDR-L1 of SEQ ID NO: 15, a CDR-L2 of SEQ ID NO: 5, and a CDR-L3 of SEQ ID NO: 16;
wherein the complex delivers the molecular payload to a cell of the CNS across the blood-brain barrier.

2. A complex comprising an anti-TfR1 antibody covalently linked to a molecular payload for treating a central nervous system (CNS) disease or disorder, wherein the anti-TfR1 antibody comprises:
(i) a heavy chain complementarity determining region 1 (CDR-H1) of SEQ ID NO: 1, a heavy chain complementarity determining region 2 (CDR-H2) of SEQ ID NO: 2, a heavy chain complementarity determining region 3 (CDR-H3) of SEQ ID NO: 3, a light chain complementarity determining region 1 (CDR-L1) of SEQ ID NO: 4, a light chain complementarity determining region 2 (CDR-L2) of SEQ ID NO: 5, and a light chain complementarity determining region 3 (CDR-L3) of SEQ ID NO: 6;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6; or
(iii) a CDR-H1 of SEQ ID NO: 12, a CDR-H2 of SEQ ID NO: 13, a CDR-H3 of SEQ ID NO: 14, a CDR-L1 of SEQ ID NO: 15, a CDR-L2 of SEQ ID NO: 5, and a CDR-L3 of SEQ ID NO: 16;
wherein the complex delivers the molecular payload to a cell of the CNS across the blood-cerebrospinal fluid barrier, optionally wherein the complex delivers the molecular payload to a cell of the CNS across the choroid plexus.

3. The complex of embodiment 1 or embodiment 2, wherein the anti-TfR1 antibody comprises a heavy chain variable region (VH) comprising an amino acid sequence of SEQ ID NO: 17 and a light chain variable region (VL) comprising an amino acid sequence of SEQ ID NO: 18.

4. The complex of any one of embodiments 1 to 3, wherein the anti-TfR1 antibody comprises a heavy chain comprising an amino acid sequence of SEQ ID NO: 19 and a light chain comprising an amino acid sequence of SEQ ID NO: 20.

5. The complex of any one of embodiments 1 to 4, wherein the anti-TfR1 antibody is a Fab.

6. The complex of any one of embodiments 1 to 5, wherein the molecular payload is configured to modulate expression of a gene associated with the CNS disease or disorder.

7. The complex of any one of embodiments 1-6, wherein the molecular payload comprises an oligonucleotide.

8. The complex of any one of embodiments 1-6, wherein the molecular payload comprises a polypeptide.

9. The complex of any one of embodiments 1-6, wherein the molecular payload comprises a small molecule.

10. The complex of any one of embodiments 1-6, wherein the molecular payload comprises a gene therapy payload, optionally wherein the gene therapy payload comprises a messenger RNA (mRNA) molecule.

11. The complex of any one of embodiments 1 to 10, wherein the anti-TfR1 antibody is covalently linked to the molecular payload via a linker comprising a structure of formula (I):

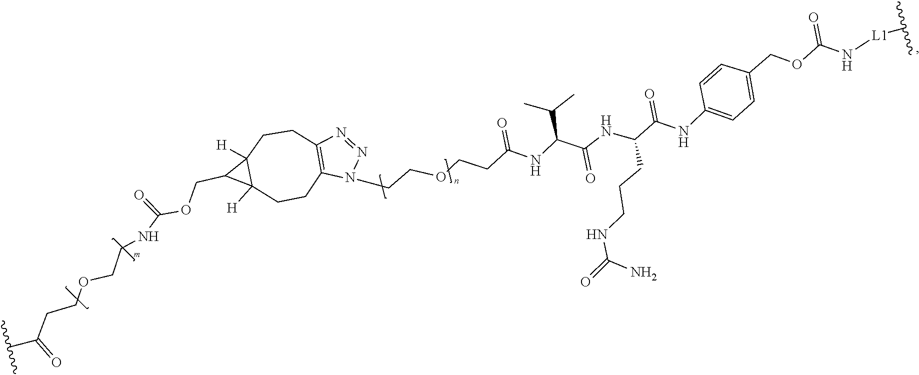

wherein n is any number from 0-10, and wherein m is any number from 0-10, optionally wherein n is 3 and/or m is 4;
and wherein L1 is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O), —OC(═O)O—, —OC(═O)N(R^{A})—, —S(O)₂NR^{A}—, —NR^{A}S(O)₂—, or a combination thereof, wherein each R^{A} is independently hydrogen or substituted or unsubstituted alkyl.

12. The complex of any one of embodiments 1 to 11, wherein the complex comprises a structure of formula (J):

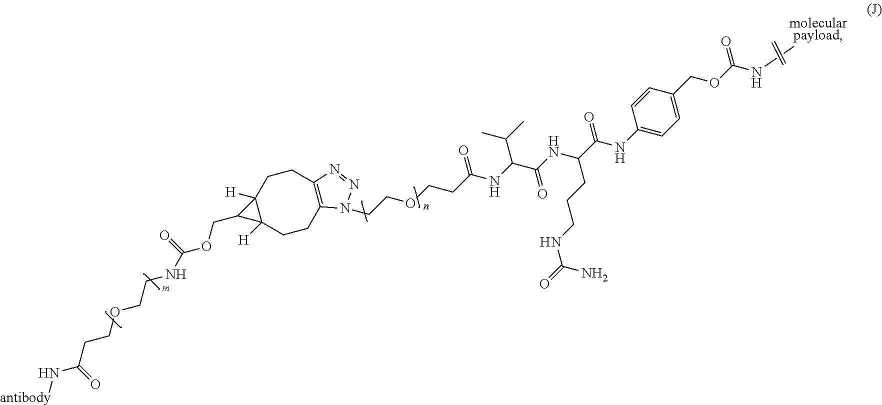

wherein n is any number from 0-10, and wherein m is any number from 0-10, optionally wherein n is 3 and/or m is 4.

13. The complex of any one of embodiments 6 to 12, wherein the gene associated with a CNS disease or disorder is DMPK, DMD, SMN, or FXN.

14. The complex of any one of embodiments 6 to 12, wherein the gene associated with a CNS disease or disorder is SOD1, C9orf72, ATXN2, or FUS.

15. The complex of any one of embodiments 6 to 12, wherein the gene associated with a CNS disease or disorder is SOD1, C9orf72, ATXN2, FUS, PIKFYVE, SYF2, or UNC13A.

16. The complex of any one of embodiments 6 to 12, wherein the gene associated with a CNS disease or disorder is LRRK2 or SNCA.

17. The complex of any one of embodiments 6 to 12, wherein the gene associated with a CNS disease or disorder is HTT or MSH3.

18. The complex of any one of embodiments 6 to 12, wherein the gene associated with a CNS disease or disorder is TREM2, APOE, MAPT, or APP.

19. The complex of any one of embodiments 6 to 12, wherein the gene associated with a CNS disease or disorder is GYS1, PrP, VLA-4, GFAP, UBE3A, LSD, or SCN9A.

20. The complex of any one of embodiments 6 to 12, wherein the gene associated with a CNS disease or disorder is SCN1A, SCN2A, SCN8A, SCN9A, CLN3, GRIA1, or PCDH19.

21. The complex of any one of embodiments 6 to 12, wherein the gene associated with a CNS disease or disorder is SCN1A, SCN2A, SCN8A, SCN9A, CLN3, GRIA1, GRIN2A, or PCDH19.

22. The complex of any one of embodiments 6 to 12, wherein the gene associated with a CNS disease or disorder is ATXN1, ATXN2, ATXN3, or MSH3.

23. The complex of any one of embodiments 6 to 12, wherein the gene associated with a CNS disease or disorder is GRN, C9orf72, MAPT, PIKFYVE, SYF2, or UNC13A.

24. The complex of any one of embodiments 6 to 12, wherein the gene associated with a CNS disease or disorder is APOE.

25. The complex of any one of embodiments 6 to 12, wherein the gene associated with a CNS disease or disorder is SCN1A.

26. The complex of any one of embodiments 6 to 12, wherein the gene associated with a CNS disease or disorder is TPP1 or CLN3.

27. The complex of any one of embodiments 6 to 12, wherein the gene associated with a CNS disease or disorder is GLB1.

28. The complex of any one of embodiments 6 to 12, wherein the gene associated with a CNS disease or disorder is ASM.

29. The complex of any one of embodiments 6 to 12, wherein the gene associated with a CNS disease or disorder is ARSA.

30. The complex of any one of embodiments 6 to 12, wherein the gene associated with a CNS disease or disorder is GALC.

31. The complex of any one of embodiments 6 to 12, wherein the gene associated with a CNS disease or disorder is HEXA.

32. The complex of any one of embodiments 6 to 12, wherein the gene associated with a CNS disease or disorder is HEXB.

33. The complex of any one of embodiments 6 to 12, wherein the gene associated with a CNS disease or disorder is GBA.

34. The complex of any one of embodiments 6 to 12, wherein the gene associated with a CNS disease or disorder is MECP2.

35. The complex of any one of embodiments 6 to 12, wherein the gene associated with a CNS disease or disorder is TOR1A, THAP1, ANO3, GNAL, KMT2B, GCH1, TH, SPR, TAF1, PRKRA, ATP1A3, SGCE, PNKD, PRRT2, SLC2A1, or ECHS1.

36. The complex of any one of embodiments 1 to 35, wherein the molecular payload comprises an oligonucleotide comprising a region of complementarity to a transcript as set forth in any one of SEQ ID NOs: 392-702, or to a target sequence of an oligonucleotide listed in any one of Tables 5-11, optionally wherein the oligonucleotide comprises an oligonucleotide structure listed in any one of Tables 5-11.

37. The complex of any one of embodiments 1 35, wherein the molecular payload comprises an oligonucleotide comprising a region of complementarity to a transcript as set forth in any one of SEQ ID NOs: 705-803 or to a target sequence of an oligonucleotide listed in any one of Tables 5-11, optionally wherein the oligonucleotide comprises an oligonucleotide structure listed in any one of Tables 5-11.

38. The complex of any one of embodiments 1 35, wherein the molecular payload comprises an oligonucleotide comprising a region of complementarity to a transcript as set forth in any one of SEQ ID NOs: 143-148, 167-169, 810-875, and 1059-1068 or to a target sequence of an oligonucleotide listed in any one of Tables 5-19, optionally wherein the oligonucleotide comprises an oligonucleotide structure listed in any one of Tables 5-19.

39. The complex of any one of embodiments 1 to 38, wherein the CNS disease or disorder is a neuromuscular disease or disorder.

40. The complex of embodiment 39, wherein the neuromuscular disease or disorder is Duchenne muscular dystrophy, myotonic dystrophy, Friedreich's ataxia, or spinal muscular atrophy.

41. The complex of any one of embodiments 1 to 38, wherein the CNS disease or disorder is amyotrophic lateral sclerosis.

42. The complex of any one of embodiments 1 to 38, wherein the CNS disease or disorder is Parkinson's disease.

43. The complex of any one of embodiments 1 to 38, wherein the CNS disease or disorder is essential tremor.

44. The complex of any one of embodiments 1 to 38, wherein the CNS disease or disorder is Huntington's disease.

45. The complex of any one of embodiments 1 to 38, wherein the CNS disease or disorder is Alzheimer's disease.

46. The complex of any one of embodiments 1 to 38, wherein the CNS disease or disorder is hereditary dystonia.

47. The complex of any one of embodiments 1 to 38, wherein the CNS disease or disorder is epilepsy.

48. The complex of any one of embodiments 1 to 38, wherein the CNS disease or disorder is a pain disorder.

49. The complex of any one of embodiments 1 to 38, wherein the CNS disease or disorder is spinocerebellar ataxia (SCA).

50. The complex of any one of embodiments 1 to 38, wherein the CNS disease or disorder is frontotemporal dementia (FTD).

51. The complex of any one of embodiments 1 to 38, wherein the CNS disease or disorder is motor neuron disease.

52. The complex of any one of embodiments 1 to 38, wherein the CNS disease or disorder is Dravet syndrome.

53. The complex of any one of embodiments 1 to 38, wherein the CNS disease or disorder is Batten disease.

54. The complex of any one of embodiments 1 to 38, wherein the CNS disease or disorder is GM1 gangliosidosis.

55. The complex of any one of embodiments 1 to 38, wherein the CNS disease or disorder is Niemann-Pick Type A.

56. The complex of any one of embodiments 1 to 38, wherein the CNS disease or disorder is metachromatic leukodystrophy.

57. The complex of any one of embodiments 1 to 38, wherein the CNS disease or disorder is Krabbe disease.

58. The complex of any one of embodiments 1 to 38, wherein the CNS disease or disorder is Tay-Sachs.

59. The complex of any one of embodiments 1 to 38, wherein the CNS disease or disorder is Sandhoff disease.

60. The complex of any one of embodiments 1 to 38, wherein the CNS disease or disorder is Gaucher disease, type II or III.

61. The complex of any one of embodiments 1 to 38, wherein the CNS disease or disorder is Rett syndrome.

62. The complex of any one of embodiments 1 to 38, wherein the CNS disease or disorder is a glycogen synthesis disorder; neurodegeneration; small fiber neuropathy; a nociception-related phenotype; Alexander disease; Angelman Syndrome; an autism-spectrum disorder; retinitis pigmentosa; isolated macular dystrophy; and/or multiple sclerosis.

63. The complex of any one of embodiments 1 to 62, wherein the molecular payload is a molecular payload disclosed in any one of paragraphs [0216]-[1208], optionally wherein the molecular payload is a molecular payload disclosed in any one of paragraphs [0296]-[0299], [0404]-[0406], [0468]-[0470], [0500]-[0502], [0535]-[0539], [0601]-[0604], [0666]-[0668], [0757]-[0759], [0779]-[0781], [0896]-[0901], [0916]-[0918], [0946]-[0948], [1049]-[1056], [1070]-[1078], [1092]-[1102], [1116]-[1124], [1138]-[1141], [1155]-[1158], [1172]-[1177], and [1191]-[1193].

64. A method of treating a CNS disease or disorder, the method comprising administering to a subject in need thereof a complex of any one of embodiments 1 to 63.

65. A method of delivering a molecular payload to the CNS of a subject (e.g., to a CNS cell of the subject), the method comprising administering to the subject a complex of any one of embodiments 1 to 63.

66. The method of embodiment 65, wherein the complex is administered to the subject intravenously.

67. The method of embodiment 65 or 66, wherein the complex is detectable in the cortex of the subject following the administration.

68. The method of any one of embodiments 65 to 67, wherein the complex is detectable in the cerebellum of the subject following the administration.

69. The method of any one of embodiments 65 to 68, wherein the complex is detectable in deep brain tissue of the subject following the administration, optionally wherein the deep brain tissue is of the thalamus, caudate nucleus and/or putamen of the subject.

70. The method of any one of embodiments 65 to 69, wherein the complex is detectable in cortical neurons, motor neurons, cells of the cerebellum, and/or choroid plexus cells of the subject following the administration.

71. The method of any one of embodiments 65 to 70, wherein the molecular payload comprises a protein, optionally wherein the protein is an enzyme.

72. The method of embodiment 71, wherein the subject has been diagnosed with or is suspected of having Batten disease, GM1 gangliosidosis, Niemann-Pick Type A, metachromatic leukodystrophy, Krabbe disease, Tay-Sachs, Sandhoff disease, or Gaucher disease.

73. The method of any one of embodiments 65 to 70, wherein the payload comprises an oligonucleotide.

74. The method of embodiment 73, wherein the subject has been diagnosed with or is suspected of having ALS, Angelman syndrome, Rett syndrome, Parkinson, lewy body dementia, Alzheimer's disease (which may or may not be associated with cerebral amyloid angiopathy (CAA) or Frontotemporal dementia), epilepsy, Alexander disease, spinal muscular atrophy, Batten disease, Huntington's disease, spinocerebellar ataxia, motor neuron disease, or Dravet syndrome.

75. The method of any one of embodiments 65 to 71 and 73, wherein the subject has been diagnosed with or is suspected of having a neuromuscular disease or disorder, optionally wherein the neuromuscular disease or disorder is: Duchenne muscular dystrophy, myotonic dystrophy, Friedreich's ataxia, or spinal muscular atrophy; amyotrophic lateral sclerosis; Parkinson's disease; Huntington's disease; or Alzheimer's disease.

76. The method of any one of embodiments 65 to 71 and 73, wherein the subject has been diagnosed with or is suspected of having essential tremor; hereditary dystonia; epilepsy; a pain disorder; a glycogen synthesis disorder; neurodegeneration; small fiber neuropathy; a nociception-related phenotype; Alexander disease; Angelman Syndrome; an autism-spectrum disorder; retinitis pigmentosa; isolated macular dystrophy; or multiple sclerosis.

77. The method of any one of embodiments 65 to 71 and 73, wherein the subject has been diagnosed with or is suspected of having spinocerebellar ataxia (SCA); frontotemporal dementia (FTD); motor neuron disease; Dravet syndrome; Batten disease; GM1 gangliosidosis; Niemann-Pick Type A; metachromatic leukodystrophy; Krabbe disease; Tay-Sachs; Sandhoff disease; Gaucher disease, type II or III; or Rett syndrome.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or (e.g., and) one or more modified nucleotides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

What is claimed is:

1. A complex comprising an anti-TfR1 antibody covalently linked to a molecular payload for treating a central nervous system (CNS) disease or disorder, wherein the anti-TfR1 antibody comprises:
(i) a heavy chain complementarity determining region 1 (CDR-H1) of SEQ ID NO: 1, a heavy chain complementarity determining region 2 (CDR-H2) of SEQ ID NO: 2, a heavy chain complementarity determining region 3 (CDR-H3) of SEQ ID NO: 3, a light chain complementarity determining region 1 (CDR-L1) of SEQ ID NO: 4, a light chain complementarity determining region 2 (CDR-L2) of SEQ ID NO: 5, and a light chain complementarity determining region 3 (CDR-L3) of SEQ ID NO: 6;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6; or
(iii) a CDR-H1 of SEQ ID NO: 12, a CDR-H2 of SEQ ID NO: 13, a CDR-H3 of SEQ ID NO: 14, a CDR-L1 of SEQ ID NO: 15, a CDR-L2 of SEQ ID NO: 5, and a CDR-L3 of SEQ ID NO: 16;
wherein the complex delivers the molecular payload to a cell of the CNS.

2. The complex of claim 1, wherein the anti-TfR1 antibody comprises a heavy chain variable region (VH) comprising an amino acid sequence of SEQ ID NO: 17 and a light chain variable region (VL) comprising an amino acid sequence of SEQ ID NO: 18.

3. The complex of claim 1 or claim 2, wherein the anti-TfR1 antibody comprises a heavy chain comprising an amino acid sequence of SEQ ID NO: 19 and a light chain comprising an amino acid sequence of SEQ ID NO: 20.

4. The complex of any one of claims 1 to 3, wherein the anti-TfR1 antibody is a Fab.

5. The complex of any one of claims 1 to 4, wherein the molecular payload is configured to modulate expression of a gene associated with the CNS disease or disorder.

6. The complex of any one of claims 1 to 5, wherein the molecular payload comprises an oligonucleotide, a polypeptide, a small molecule, or a gene therapy payload, optionally wherein the gene therapy payload comprises a messenger RNA (mRNA) molecule.

7. The complex of any one of claims 1 to 6, wherein the anti-TfR1 antibody is covalently linked to the molecular payload via a linker comprising a structure of formula (I): 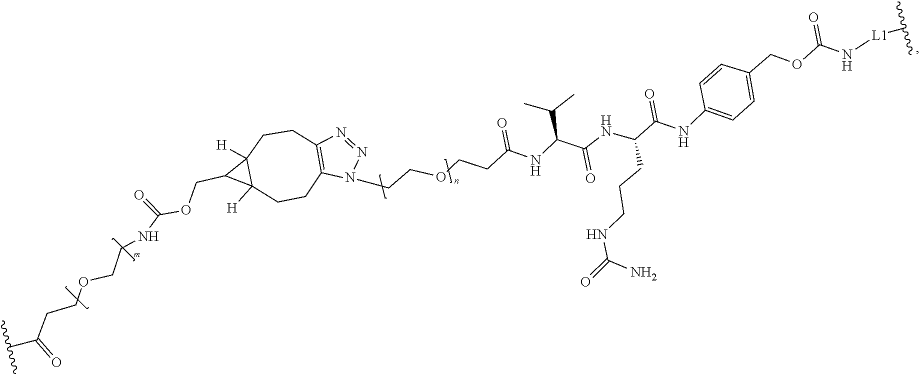
wherein n is any number from 0-10, and wherein m is any number from 0-10, optionally wherein n is 3 and/or m is 4;
and wherein L1 is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, S(O)₂NR^{A}—, —NR^{A}S(O)₂—, or a combination thereof, wherein each R^{A} is independently hydrogen or substituted or unsubstituted alkyl.

8. The complex of any one of claims 1 to 7, wherein the complex comprises a structure of formula (J): 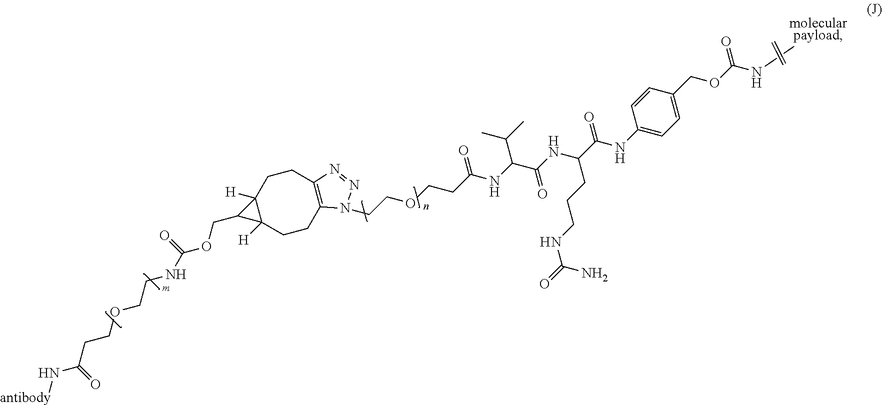
wherein n is any number from 0-10, and wherein m is any number from 0-10, optionally wherein n is 3 and/or m is 4.

9. The complex of any one of claims 1 to 8, wherein the complex delivers the molecular payload to the cell of the CNS across the blood-brain barrier.

10. The complex of any one of claims 1 to 8, wherein the complex delivers the molecular payload to the cell of the CNS across the choroid plexus.

11. The complex of any one of claims 5 to 10, wherein the gene associated with a CNS disease or disorder is:
(i) DMPK, DMD, SMN, or FXN;
(ii) SOD1, C9orf72, ATXN2, or FUS;
(iii) LRRK2 or SNCA;
(iv) HTT or MSH3;
(v) TREM2, APOE, MAPT, or APP;
(vi) GYS1, PrP, VLA-4, GFAP, UBE3A, LSD, or SCN9A; or
(vii) SCN1A, SCN2A, SCN8A, SCN9A, CLN3, GRIA1, or PCDH19.

12. The complex of any one of claims 5 to 10, wherein the gene associated with a CNS disease or disorder is: TOR1A, THAP1, ANO3, GNAL, KMT2B, GCH1, TH, SPR, TAF1, PRKRA, ATP1A3, SGCE, PNKD, PRRT2, SLC2A1, or ECHS1.

13. The complex of any one of claims 5 to 10, wherein the gene associated with a CNS disease or disorder is:
(i) PIKFYVE, SYF2, or UNC13A;
(ii) GRIN2A;
(iii) ATXN1, ATXN2, ATXN3, or MSH3;
(iv) GRN, C9orf72, MAPT, PIKFYVE, SYF2, or UNC13A;
(v) TPP1 or CLN3; or
(vi) APOE, SCN1A, GLB1, ASM, ARSA, GALC, HEXA, HEXB, GBA, or MECP2.

14. The complex of any one of claims 1 to 13, wherein the molecular payload comprises an oligonucleotide comprising a region of complementarity to a transcript as set forth in any one of SEQ ID NOs: 392-702, or to a target sequence of an oligonucleotide listed in any one of Tables 5-19, optionally wherein the oligonucleotide comprises an oligonucleotide structure listed in any one of Tables 5-19.

15. The complex of any one of claims 1 to 13, wherein the molecular payload comprises an oligonucleotide comprising a region of complementarity to a transcript as set forth in any one of SEQ ID NOs: 705-803, or to a target sequence of an oligonucleotide listed in any one of Tables 5-19, optionally wherein the oligonucleotide comprises an oligonucleotide structure listed in any one of Tables 5-19.

16. The complex of any one of claims 1 to 13, wherein the molecular payload comprises an oligonucleotide comprising a region of complementarity to a transcript as set forth in any one of SEQ ID NOs: 143-148, 167-169, 810-875, and 1059-1068, or to a target sequence of an oligonucleotide listed in any one of Tables 5-19, optionally wherein the oligonucleotide comprises an oligonucleotide structure listed in any one of Tables 5-19.

17. The complex of any one of claims 1 to 16, wherein the CNS disease or disorder is a neuromuscular disease or disorder, optionally wherein the neuromuscular disease or disorder is: Duchenne muscular dystrophy, myotonic dystrophy, Friedreich's ataxia, or spinal muscular atrophy.

18. The complex of any one of claims 1 to 16, wherein the CNS disease or disorder is:
(i) amyotrophic lateral sclerosis;
(ii) Parkinson's disease;
(iii) essential tremor;
(iv) Huntington's disease;
(v) Alzheimer's disease;
(vi) hereditary dystonia;
(vii) epilepsy;
(viii) a pain disorder; or
(ix) a glycogen synthesis disorder; neurodegeneration; small fiber neuropathy; a nociception-related phenotype; Alexander disease; Angelman Syndrome; an autism-spectrum disorder; retinitis pigmentosa; isolated macular dystrophy; and/or multiple sclerosis.

19. The complex of any one of claims 1 to 16, wherein the CNS disease or disorder is:
(i) spinocerebellar ataxia (SCA);
(ii) frontotemporal dementia (FTD);
(iii) motor neuron disease;
(iv) Dravet syndrome;
(v) Batten disease;
(vi) GM1 gangliosidosis;
(vii) Niemann-Pick Type A;
(viii) metachromatic leukodystrophy;
(ix) Krabbe disease;
(x) Tay-Sachs;
(xi) Sandhoff disease;
(xii) Gaucher disease, type II or III; or
(xiii) Rett syndrome.

20. The complex of any one of claims 1 to 19, wherein the molecular payload is a molecular payload disclosed in any one of paragraphs [0216]-[1208], optionally wherein the molecular payload is a molecular payload disclosed in any one of paragraphs [0296]-[0299], [0404]-[0406], [0468]-[0470], [0500]-[0502], [0535]-[0539], [0601]-[0604], [0666]-[0668], [0757]-[0759], [0779]-[0781], [0896]-[0901], [0916]-[0918], [0946]-[0948], [1049]-[1056], [1070]-[1078], [1092]-[1102], [1116]-[1124], [1138]-[1141], [1155]-[1158], [1172]-[1177], and [1191]-[1193].

21. A method of treating a CNS disease or disorder, the method comprising administering to a subject in need thereof a complex of any one of claims 1 to 20.

22. A method of delivering a molecular payload to the CNS of a subject, the method comprising administering to the subject a complex of any one of claims 1 to 20.

23. The method of claim 22, wherein the complex is administered to the subject intravenously.

24. The method of claim 22 or 23, wherein the complex is detectable in the cortex of the subject following the administration.

25. The method of any one of claims 22 to 24, wherein the complex is detectable in the cerebellum of the subject following the administration.

26. The method of any one of claims 22 to 25, wherein the complex is detectable in deep brain tissue of the subject following the administration, optionally wherein the deep brain tissue is of the thalamus, caudate nucleus and/or putamen of the subject.

27. The method of any one of claims 22 to 26, wherein the complex is detectable in cortical neurons, motor neurons, cells of the cerebellum, and/or choroid plexus cells of the subject following the administration.

28. The method of any one of claims 22 to 27, wherein the molecular payload comprises a protein, optionally wherein the protein is an enzyme.

29. The method of claim 28, wherein the subject has been diagnosed with or is suspected of having Batten disease, GM1 gangliosidosis, Niemann-Pick Type A, metachromatic leukodystrophy, Krabbe disease, Tay-Sachs, Sandhoff disease, or Gaucher disease.

30. The method of any one of claims 22 to 27, wherein the payload comprises an oligonucleotide.

31. The method of claim 30, wherein the subject has been diagnosed with or is suspected of having ALS, Angelman syndrome, Rett syndrome, Parkinson, lewy body dementia, Alzheimer's disease (which may or may not be associated with cerebral amyloid angiopathy (CAA) or Frontotemporal dementia), epilepsy, Alexander disease, spinal muscular atrophy, Batten disease, Huntington's disease, spinocerebellar ataxia, motor neuron disease, or Dravet syndrome.
